SEQUENCE LISTING (246 sequences)

SEQ ID NO: 1 - protein, 4 aa, Artificial Sequence
  Description of Artificial Sequence Synthetic peptide
LRGS

SEQ ID NO: 2 - protein, 23 aa, Artificial Sequence
  Description of Artificial Sequence Synthetic peptide
  MOD_RES (12)..(18): Any amino acid
FQCRICMRNFSXXXXXXXHIRTH

SEQ ID NO: 3 - protein, 23 aa, Artificial Sequence
  Description of Artificial Sequence Synthetic peptide
  MOD_RES (12)..(18): Any amino acid
FACDICGRKFAXXXXXXXHTKIH

SEQ ID NO: 4 - DNA, 15 bp, Unknown
  Description of Unknown EGFPDP2 DLR target sequence
ggggaggacgcggtg

SEQ ID NO: 5 - protein, 139 aa, Artificial Sequence
  Description of Artificial Sequence Synthetic polypeptide
FQCRICMRNFSRSSALTRHIRTHTGEKPFACDICGRKFARSDTLTRHTKIHTGSQKPFQCRICMRNFSDRSNLTRHIRTHTGEKPFACDICGRKFARSDNLTRHTKIHTGSQKPFQCRICMRNFSRSDHLTRHIRTHTG

SEQ ID NO: 6 - DNA, 18 bp, Unknown
  Description of Unknown EGFPDP2 DLR target sequence
gtggagctggacggggac

SEQ ID NO: 7 - protein, 165 aa, Artificial Sequence
  Description of Artificial Sequence Synthetic polypeptide
FQCRICMRNFSDRSNLTRHIRTHTGEKPFACDICGRKFARSDHLTRHTKIHTGSQKPFQCRICMRNFSDRSNLTRHIRTHTGEKPFACDICGRKFARSDSLSEHTKIHTGSQKPFQCRICMRNFSRSSNLTRHIRTHTGEKPFACDICGRKFARSDSLTRHTKIH

SEQ ID NO: 8 - DNA, 27 bp, Homo sapiens
gcggccgcctggtgcagtaccgcggcg

SEQ ID NO: 9 - protein, 261 aa, Artificial Sequence
  Description of Artificial Sequence Synthetic polypeptide
MAAMAERPFQCRICMRNFSRSSDLTRHIRTHTGEKPFACDICGRKFARSDTLTRHTKIHTGSQKPFQCRICMRNFSQSGDLSEHIRTHTGEKPFACDICGRKFATSGHLTTHTKIHTGSQKPFQCRICMRNFSDSSHLTTHIRTHTGEKPFACDICGRKFARSSHLTTHTKIHTGSQKPFQCRICMRNFSDRSDLTRHIRTHTGEKPFACDICGRKFADRSDLTRHTKIHTGSQKPFQCRICMRNFSRSDTLTRHIRTHTG

SEQ ID NO: 10 - DNA, 33 bp, Unknown
  Description of Unknown ApoE codon 158 site DLR target sequence
ctggcagtgtaccaggccggggcccgcgagggc

SEQ ID NO: 11 - protein, 318 aa, Artificial Sequence
  Description of Artificial Sequence Synthetic polypeptide
MAAMAERPFQCRICMRNFSDRSHLTRHIRTHTGEKPFACDICGRKFARSDNLTRHTKIHTGSQKPFQCRICMRNFSDSSHLSEHIRTHTGEKPFACDICGRKFADRSDLTRHTKIHTGSQKPFQCRICMRNFSRSDHLTRHIRTHTGEKPFACDICGRKFADRSDLTRHTKIHTGSQKPFQCRICMRNFSRSDNLSEHIRTHTGEKPFACDICGRKFAESSNLTTHTKIHTGSQKPFQCRICMRNFSRSSSLTRHIRTHTGEKPFACDICGRKFAQSSDLTRHTKIHTGSQKPFQCRICMRNFSRSDSLSEHIRTHTG

SEQ ID NO: 12 - protein, 1367 aa, Artificial Sequence
  Description of Artificial Sequence Synthetic polypeptide
DKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 13 - protein, 10 aa, Artificial Sequence
  Description of Artificial Sequence Synthetic peptide
LRQKDAARGS

SEQ ID NO: 14 - protein, 36 aa, Artificial Sequence
  Description of Artificial Sequence Synthetic polypeptide
GGGGGSGGGGGSGGGGGSGGGGGSGGGGGSGGGGGS

SEQ ID NO: 15 - protein, 22 aa, Artificial Sequence
  Description of Artificial Sequence Synthetic peptide
MVSKGEELFTASSPSSWSWTGT

SEQ ID NO: 16 - protein, 239 aa, Artificial Sequence
  Description of Artificial Sequence Synthetic polypeptide
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 17 - DNA, 5720 bp, Artificial Sequence
  Description of Artificial Sequence Synthetic polynucleotide
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttatggtgagcaagggcgaggagctgttcaccgcgtcctccccatcctcgtggagctggacggggacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaactcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggtccctttagggttccgatttagtgctttacggcacctcgaccccaaaaaacttgattagggtgatggttcacgtacctagaagttcctattccgaagttcctattctctagaaagtataggaacttccttggccaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagcacgtactacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 18 - DNA, 3604 bp, Artificial Sequence
  Description of Artificial Sequence Synthetic polynucleotide
gactcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttatggactacaaagaccatgacggtgattataaagatcatgacatcgattacaaggatgacgatgacaagatggcccccaagaagaagaggaaggtgggcattcacggggtaccggcggcgatggccgagcggcccttccagtgcaggatctgtatgcgcaacttttctcgttcttctgctcttactcgtcacatcagaacccatacaggcgaaaagcctttcgcctgcgacatttgtgggagaaaatttgctcgttctgatactcttactcgtcataccaagatccacaccggctctcagaaaccattccagtgccgcatttgtatgcggaatttttccgatcgttctaatcttactcgtcatatccgcactcacaccggagagaagccctttgcttgcgacatttgtggcaggaaatttgctcgttctgataatcttactcgtcacactaagatccatactgggtcacagaaacctttccagtgccggatttgtatgagaaactttagccgttctgatcatcttactcgtcacatcagaacacatactgggctgagaggatccaattctggtgatcctcggagacacagtctgggcggttctcgtaaacccgatctgattgcctataaaaactttgatctgctggtcattgttcttaagccttgagcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttctt

SEQ ID NO: 19 - protein, 33 aa, Artificial Sequence
  Description of Artificial Sequence Synthetic polypeptide
NSGDPRRHSLGGSRKPDLIAYKNFDLLVIVLKP

SEQ ID NO: 20 - DNA, 102 bp, Artificial Sequence
  Description of Artificial Sequence Synthetic polynucleotide
aattctggtgatcctcggagacacagtctgggcggttctcgtaaacccgatctgattgcctataaaaactttgatctgctggtcattgttcttaagccttga

SEQ ID NO: 21 - DNA, 3835 bp, Artificial Sequence
  Description of Artificial Sequence Synthetic polynucleotide
gactcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttatggcggcgatggccgagcggcccttccagtgcaggatctgtatgcgcaacttttctcggtcctccgacctgacccggcacatcagaacccatacaggcgaaaagcctttcgcctgcgacatttgtgggagaaaatttgctcggtccgacaccctgacccggcataccaagatccacaccggctctcagaaaccattccagtgccgcatttgtatgcggaatttttcccagtccggcgacctgtccgagcatatccgcactcacaccggagagaagccctttgcttgcgacatttgtggcaggaaatttgctacctccggccacctgaccacccacactaagatccatactgggtcacagaaacctttccagtgccggatttgtatgagaaactttagcgactcctcccacctgaccacccacatcagaacccatacaggcgaaaagcctttcgcctgcgacatttgtgggagaaaatttgctcggtcctcccacctgaccacccataccaagatccacaccggctctcagaaaccattccagtgccgcatttgtatgcggaatttttccgaccggtccgacctgacccggcatatccgcactcacaccggagagaagccctttgcttgcgacatttgtggcaggaaatttgctgaccggtccgacctgacccggcacactaagatccatactgggtcacagaaacctttccagtgccggatttgtatgagaaactttagccggtccgacaccctgacccggcacatcagaacacatactgggctgagaggatccaattctggtgatcctcggagacacagtctgggcggttctcgtaaacccgatctgattgcctataaaaactttgatctgctggtcattgttcttaagccttgagcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttctt

SEQ ID NO: 22 - DNA, 129 bp, Artificial Sequence
  Description of Artificial Sequence Synthetic polynucleotide
ccccggtggcggaggagacgcgggcacggctgtccaaggagctgcaggcggcgcaggcccggctgggcgcggacatggaggacgtgcgcggccgcctggtgcagtaccgcggcgaggtgcaggccatgc

SEQ ID NO: 23 - DNA, 150 bp, Artificial Sequence
  Description of Artificial Sequence Synthetic polynucleotide
ccccggtggcggaggagacgcgggcacggctgtccaaggagctgcaggcggcgcaggcccggctgggcgcggacatggaggacgtgcgcggccgcctggtgcagtaccgcggcgaggtgcaggccatgctcggccagagcaccgaggagc

SEQ ID NO: 24 - DNA, 17 bp, Artificial Sequence
  Description of Artificial Sequence Synthetic primer
ctgcaggcggcgcaggc

SEQ ID NO: 25 - DNA, 20 bp, Artificial Sequence
  Description of Artificial Sequence Synthetic primer
ctcctcggtgctctggccga

SEQ ID NO: 26 - DNA, 53 bp, Artificial Sequence
  Description of Artificial Sequence Synthetic primer
acactctttccctacacgacgctcttccgatcttcggccagagcaccgaggag

SEQ ID NO: 27 - DNA, 50 bp, Artificial Sequence
  Description of Artificial Sequence Synthetic primer
gactggagttcagacgtgtgctcttccgatctgcatggcctgcacctcgc

SEQ ID NO: 28 - DNA, 4006 bp, Artificial Sequence
  Description of Artificial Sequence Synthetic polynucleotide
gactcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttatggcggcgatggccgagcggcccttccagtgcaggatctgtatgcgcaacttttctgaccggtcccacctgacccggcacatcagaacccatacaggcgaaaagcctttcgcctgcgacatttgtgggagaaaatttgctcggtccgacaacctgacccggcataccaagatccacaccggctctcagaaaccattccagtgccgcatttgtatgcggaatttttccgactcctcccacctgtccgagcatatccgcactcacaccggagagaagccctttgcttgcgacatttgtggcaggaaatttgctgaccggtccgacctgacccggcacactaagatccatactgggtcacagaaacctttccagtgccggatttgtatgagaaactttagccggtccgaccacctgacccggcacatcagaacccatacaggcgaaaagcctttcgcctgcgacatttgtgggagaaaatttgctgaccggtccgacctgacccggcataccaagatccacaccggctctcagaaaccattccagtgccgcatttgtatgcggaatttttcccggtccgacaacctgtccgagcatatccgcactcacaccggagagaagccctttgcttgcgacatttgtggcaggaaatttgctgagtcctccaacctgaccacccataccaagatccacaccggctctcagaaaccattccagtgccgcatttgtatgcggaatttttcccggtcctcctccctgacccggcatatccgcactcacaccggagagaagccctttgcttgcgacatttgtggcaggaaatttgctcagtcctccgacctgacccggcacactaagatccatactgggtcacagaaacctttccagtgccggatttgtatgagaaactttagccggtccgactccctgtccgagcacatcagaacacatactgggctgagaggatccaattctggtgatcctcggagacacagtctgggcggttctcgtaaacccgatctgattgcctataaaaactttgatctgctggtcattgttcttaagccttgagcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttctt

SEQ ID NO: 29 - DNA, 150 bp, Artificial Sequence
  Description of Artificial Sequence Synthetic polynucleotide
gcgggtgcgcctcgcctcccacctgcgcaagctgcgtaagcggctcctccgcgatgccgatgacctgcagaagtgcctggcagtgtaccaggccggggcccgcgagggcgccgagcgcggcctcagcgccatccgcgagcgcctggggcc

SEQ ID NO: 30 - DNA, 150 bp, Artificial Sequence
  Description of Artificial Sequence Synthetic polynucleotide
ggccccaggcgctcgcggatggcgctgaggccgcgctcggcgccctcgcgggccccggcctggtacactgccaggcacttctgcaggtcatcggcatcgcggaggagccgcttacgcagcttgcgcaggtgggaggcgaggcgcacccgc

SEQ ID NO: 31 - DNA, 200 bp, Artificial Sequence
  Description of Artificial Sequence Synthetic polynucleotide
ccggctgggcgcggacatggaggacgtgcgcggccgcctggtgcagtaccgcggcgaggtgcaggccatgctcggccagagcaccgaggagctgcgggtgcgcctcgcctcccacctgcgcaagctgcgtaagcggctcctccgcgatgccgatgacctgcagaagtgcctggcagtgtaccaggccggggcccgcgagg

SEQ ID NO: 32 - DNA, 200 bp, Artificial Sequence
  Description of Artificial Sequence Synthetic polynucleotide
cctcgcgggccccggcctggtacactgccaggcacttctgcaggtcatcggcatcgcggaggagccgcttacgcagcttgcgcaggtgggaggcgaggcgcacccgcagctcctcggtgctctggccgagcatggcctgcacctcgccgcggtactgcaccaggcggccgcgcacgtcctccatgtccgcgcccagccgg

SEQ ID NO: 33 - DNA, 150 bp, Artificial Sequence
  Description of Artificial Sequence Synthetic polynucleotide
ccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagc

SEQ ID NO: 34 - protein, 298 aa, Artificial Sequence
  Description of Artificial Sequence Synthetic polypeptide
MAAMAERPFQCRICMRNFSRSSDLTRHIRTHTGEKPFACDICGRKFARSDTLTRHTKIHTGSQKPFQCRICMRNFSQSGDLSEHIRTHTGEKPFACDICGRKFATSGHLTTHTKIHTGSQKPFQCRICMRNFSDSSHLTTHIRTHTGEKPFACDICGRKFARSSHLTTHTKIHTGSQKPFQCRICMRNFSDRSDLTRHIRTHTGEKPFACDICGRKFADRSDLTRHTKIHTGSQKPFQCRICMRNFSRSDTLTRHIRTHTGLRGSNSGDPRRHSLGGSRKPDLIAYKNFDLLVIQLKP

SEQ ID NO: 35 - protein, 298 aa, Artificial Sequence
  Description of Artificial Sequence Synthetic polypeptide
MAAMAERPFQCRICMRNFSRSSDLTRHIRTHTGEKPFACDICGRKFARSDTLTRHTKIHTGSQKPFQCRICMRNFSQSGDLSEHIRTHTGEKPFACDICGRKFATSGHLTTHTKIHTGSQKPFQCRICMRNFSDSSHLTTHIRTHTGEKPFACDICGRKFARSSHLTTHTKIHTGSQKPFQCRICMRNFSDRSDLTRHIRTHTGEKPFACDICGRKFADRSDLTRHTKIHTGSQKPFQCRICMRNFSRSDTLTRHIRTHTGLRGSNSGDPRRHSLGGSRKPDLIAYKNFDLLVINLKP

SEQ ID NO: 36 - protein, 298 aa, Artificial Sequence
  Description of Artificial Sequence Synthetic polypeptide
MAAMAERPFQCRICMRNFSRSSDLTRHIRTHTGEKPFACDICGRKFARSDTLTRHTKIHTGSQKPFQCRICMRNFSQSGDLSEHIRTHTGEKPFACDICGRKFATSGHLTTHTKIHTGSQKPFQCRICMRNFSDSSHLTTHIRTHTGEKPFACDICGRKFARSSHLTTHTKIHTGSQKPFQCRICMRNFSDRSDLTRHIRTHTGEKPFACDICGRKFADRSDLTRHTKIHTGSQKPFQCRICMRNFSRSDTLTRHIRTHTGLRGSNSGDPRRHSLGGSRKPDLIAYKNFDLLVISLKP

SEQ ID NO: 37 - protein, 298 aa, Artificial Sequence
  Description of Artificial Sequence Synthetic polypeptide
MAAMAERPFQCRICMRNFSRSSDLTRHIRTHTGEKPFACDICGRKFARSDTLTRHTKIHTGSQKPFQCRICMRNFSQSGDLSEHIRTHTGEKPFACDICGRKFATSGHLTTHTKIHTGSQKPFQCRICMRNFSDSSHLTTHIRTHTGEKPFACDICGRKFARSSHLTTHTKIHTGSQKPFQCRICMRNFSDRSDLTRHIRTHTGEKPFACDICGRKFADRSDLTRHTKIHTGSQKPFQCRICMRNFSRSDTLTRHIRTHTGLRGSNSGDPRRHSLGGSRKPDLIAYKNFDLLVITLKP

SEQ ID NO: 38 - protein, 298 aa, Artificial Sequence
  Description of Artificial Sequence Synthetic polypeptide
MAAMAERPFQCRICMRNFSRSSDLTRHIRTHTGEKPFACDICGRKFARSDTLTRHTKIHTGSQKPFQCRICMRNFSQSGDLSEHIRTHTGEKPFACDICGRKFATSGHLTTHTKIHTGSQKPFQCRICMRNFSDSSHLTTHIRTHTGEKPFACDICGRKFARSSHLTTHTKIHTGSQKPFQCRICMRNFSDRSDLTRHIRTHTGEKPFACDICGRKFADRSDLTRHTKIHTGSQKPFQCRICMRNFSRSDTLTRHIRTHTGLRGSNSGDPRRHSLGGSRKPDLIAYKNFDLLVIALKP

SEQ ID NO: 39 - protein, 298 aa, Artificial Sequence
  Description of Artificial Sequence Synthetic polypeptide
MAAMAERPFQCRICMRNFSRSSDLTRHIRTHTGEKPFACDICGRKFARSDTLTRHTKIHTGSQKPFQCRICMRNFSQSGDLSEHIRTHTGEKPFACDICGRKFATSGHLTTHTKIHTGSQKPFQCRICMRNFSDSSHLTTHIRTHTGEKPFACDICGRKFARSSHLTTHTKIHTGSQKPFQCRICMRNFSDRSDLTRHIRTHTGEKPFACDICGRKFADRSDLTRHTKIHTGSQKPFQCRICMRNFSRSDTLTRHIRTHTGLRGSNSGDPRRHSLGGSRKPDLIAYKNFDLLVILLKP

SEQ ID NO: 40 - protein, 298 aa, Artificial Sequence
  Description of Artificial Sequence Synthetic polypeptide
MAAMAERPFQCRICMRNFSRSSDLTRHIRTHTGEKPFACDICGRKFARSDTLTRHTKIHTGSQKPFQCRICMRNFSQSGDLSEHIRTHTGEKPFACDICGRKFATSGHLTTHTKIHTGSQKPFQCRICMRNFSDSSHLTTHIRTHTGEKPFACDICGRKFARSSHLTTHTKIHTGSQKPFQCRICMRNFSDRSDLTRHIRTHTGEKPFACDICGRKFADRSDLTRHTKIHTGSQKPFQCRICMRNFSRSDTLTRHIRTHTGLRGSNSGDPRRHSLGGSRKPDLIAYKNFDLLVIILKP

SEQ ID NO: 41 - protein, 298 aa, Artificial Sequence
  Description of Artificial Sequence Synthetic polypeptide
MAAMAERPFQCRICMRNFSRSSDLTRHIRTHTGEKPFACDICGRKFARSDTLTRHTKIHTGSQKPFQCRICMRNFSQSGDLSEHIRTHTGEKPFACDICGRKFATSGHLTTHTKIHTGSQKPFQCRICMRNFSDSSHLTTHIRTHTGEKPFACDICGRKFARSSHLTTHTKIHTGSQKPFQCRICMRNFSDRSDLTRHIRTHTGEKPFACDICGRKFADRSDLTRHTKIHTGSQKPFQCRICMRNFSRSDTLTRHIRTHTGLRGSNSGDPRRHSLGGSRKPDLIAYKNFDLLVIHLKP

SEQ ID NO: 42 - protein, 298 aa, Artificial Sequence
  Description of Artificial Sequence Synthetic polypeptide
MAAMAERPFQCRICMRNFSRSSDLTRHIRTHTGEKPFACDICGRKFARSDTLTRHTKIHTGSQKPFQCRICMRNFSQSGDLSEHIRTHTGEKPFACDICGRKFATSGHLTTHTKIHTGSQKPFQCRICMRNFSDSSHLTTHIRTHTGEKPFACDICGRKFARSSHLTTHTKIHTGSQKPFQCRICMRNFSDRSDLTRHIRTHTGEKPFACDICGRKFADRSDLTRHTKIHTGSQKPFQCRICMRNFSRSDTLTRHIRTHTGLRGSNSGDPRRHSLGGSRKPDLIAYKNFDLLVIRLKP

SEQ ID NO: 43 - protein, 298 aa, Artificial Sequence
  Description of Artificial Sequence Synthetic polypeptide
MAAMAERPFQCRICMRNFSRSSDLTRHIRTHTGEKPFACDICGRKFARSDTLTRHTKIHTGSQKPFQCRICMRNFSQSGDLSEHIRTHTGEKPFACDICGRKFATSGHLTTHTKIHTGSQKPFQCRICMRNFSDSSHLTTHIRTHTGEKPFACDICGRKFARSSHLTTHTKIHTGSQKPFQCRICMRNFSDRSDLTRHIRTHTGEKPFACDICGRKFADRSDLTRHTKIHTGSQKPFQCRICMRNFSRSDTLTRHIRTHTGLRGSNSGDPRRHSLGGSRKPDLIAYKNFDLLVIKLKP

SEQ ID NO: 44 - protein, 298 aa, Artificial Sequence
  Description of Artificial Sequence Synthetic polypeptide
MAAMAERPFQCRICMRNFSRSSDLTRHIRTHTGEKPFACDICGRKFARSDTLTRHTKIHTGSQKPFQCRICMRNFSQSGDLSEHIRTHTGEKPFACDICGRKFATSGHLTTHTKIHTGSQKPFQCRICMRNFSDSSHLTTHIRTHTGEKPFACDICGRKFARSSHLTTHTKIHTGSQKPFQCRICMRNFSDRSDLTRHIRTHTGEKPFACDICGRKFADRSDLTRHTKIHTGSQKPFQCRICMRNFSRSDTLTRHIRTHTGLRGSNSGDPRRHSLGGSRKPDLIAYKNFDLLVIMLKP

SEQ ID NO: 45 - protein, 298 aa, Artificial Sequence
  Description of Artificial Sequence Synthetic polypeptide
MAAMAERPFQCRICMRNFSRSSDLTRHIRTHTGEKPFACDICGRKFARSDTLTRHTKIHTGSQKPFQCRICMRNFSQSGDLSEHIRTHTGEKPFACDICGRKFATSGHLTTHTKIHTGSQKPFQCRICMRNFSDSSHLTTHIRTHTGEKPFACDICGRKFARSSHLTTHTKIHTGSQKPFQCRICMRNFSDRSDLTRHIRTHTGEKPFACDICGRKFADRSDLTRHTKIHTGSQKPFQCRICMRNFSRSDTLTRHIRTHTGLRGSNSGDPRRHSLGGSRKPNLIAYKNFDLLVIELKP

SEQ ID NO: 46 - protein, 298 aa, Artificial Sequence
  Description of Artificial Sequence Synthetic polypeptide
MAAMAERPFQCRICMRNFSRSSDLTRHIRTHTGEKPFACDICGRKFARSDTLTRHTKIHTGSQKPFQCRICMRNFSQSGDLSEHIRTHTGEKPFACDICGRKFATSGHLTTHTKIHTGSQKPFQCRICMRNFSDSSHLTTHIRTHTGEKPFACDICGRKFARSSHLTTHTKIHTGSQKPFQCRICMRNFSDRSDLTRHIRTHTGEKPFACDICGRKFADRSDLTRHTKIHTGSQKPFQCRICMRNFSRSDTLTRHIRTHTGLRGSNSGDPRRHSLGGSRKPALIAYKNFDLLVIELKP

SEQ ID NO: 47 - protein, 302 aa, Artificial Sequence
  Description of Artificial Sequence Synthetic polypeptide
MAAMAERPFQCRICMRNFSRSSDLTRHIRTHTGEKPFACDICGRKFARSDTLTRHTKIHTGSQKPFQCRICMRNFSQSGDLSEHIRTHTGEKPFACDICGRKFATSGHLTTHTKIHTGSQKPFQCRICMRNFSDSSHLTTHIRTHTGEKPFACDICGRKFARSSHLTTHTKIHTGSQKPFQCRICMRNFSDRSDLTRHIRTHTGEKPFACDICGRKFADRSDLTRHTKIHTGSQKPFQCRICMRNFSRSDTLTRHIRTHTGLRGSNSGDPRRHSLGGSRKPDGAIYTVGSPIDYGVIVVTKP

SEQ ID NO: 48 - protein, 301 aa, Artificial Sequence
  Description of Artificial Sequence Synthetic polypeptide
MAAMAERPFQCRICMRNFSRSSDLTRHIRTHTGEKPFACDICGRKFARSDTLTRHTKIHTGSQKPFQCRICMRNFSQSGDLSEHIRTHTGEKPFACDICGRKFATSGHLTTHTKIHTGSQKPFQCRICMRNFSDSSHLTTHIRTHTGEKPFACDICGRKFARSSHLTTHTKIHTGSQKPFQCRICMRNFSDRSDLTRHIRTHTGEKPFACDICGRKFADRSDLTRHTKIHTGSQKPFQCRICMRNFSRSDTLTRHIRTHTGLRGSNSGDPRRHSLGGSRKPDFTLYKPSEPNKKIAIVIKP

SEQ ID NO: 49 - protein, 298 aa, Artificial Sequence
  Description of Artificial Sequence Synthetic polypeptide
MAAMAERPFQCRICMRNFSRSSDLTRHIRTHTGEKPFACDICGRKFARSDTLTRHTKIHTGSQKPFQCRICMRNFSQSGDLSEHIRTHTGEKPFACDICGRKFATSGHLTTHTKIHTGSQKPFQCRICMRNFSDSSHLTTHIRTHTGEKPFACDICGRKFARSSHLTTHTKIHTGSQKPFQCRICMRNFSDRSDLTRHIRTHTGEKPFACDICGRKFADRSDLTRHTKIHTGSQKPFQCRICMRNFSRSDTLTRHIRTHTGLRGSNSGDPRRHSLGGSRKPDGLLWDDDCAIILVSKP

SEQ ID NO: 50 - protein, 302 aa, Artificial Sequence
  Description of Artificial Sequence Synthetic polypeptide
MAAMAERPFQCRICMRNFSRSSDLTRHIRTHTGEKPFACDICGRKFARSDTLTRHTKIHTGSQKPFQCRICMRNFSQSGDLSEHIRTHTGEKPFACDICGRKFATSGHLTTHTKIHTGSQKPFQCRICMRNFSDSSHLTTHIRTHTGEKPFACDICGRKFARSSHLTTHTKIHTGSQKPFQCRICMRNFSDRSDLTRHIRTHTGEKPFACDICGRKFADRSDLTRHTKIHTGSQKPFQCRICMRNFSRSDTLTRHIRTHTGLRGSNSGDPRRHSLGGSRKPDHIYQLVYNSTDTLLLIVSKP

SEQ ID NO: 51 - protein, 303 aa, Artificial Sequence
  Description of Artificial Sequence Synthetic polypeptide
MAAMAERPFQCRICMRNFSRSSDLTRHIRTHTGEKPFACDICGRKFARSDTLTRHTKIHTGSQKPFQCRICMRNFSQSGDLSEHIRTHTGEKPFACDICGRKFATSGHLTTHTKIHTGSQKPFQCRICMRNFSDSSHLTTHIRTHTGEKPFACDICGRKFARSSHLTTHTKIHTGSQKPFQCRICMRNFSDRSDLTRHIRTHTGEKPFACDICGRKFADRSDLTRHTKIHTGSQKPFQCRICMRNFSRSDTLTRHIRTHTGLRGSNSGDPRRHSLGGSRKPDHIYIFNDDNNTKNGLIIVSKP

SEQ ID NO: 52 - protein, 303 aa, Artificial Sequence
  Description of Artificial Sequence Synthetic polypeptide
MAAMAERPFQCRICMRNFSRSSDLTRHIRTHTGEKPFACDICGRKFARSDTLTRHTKIHTGSQKPFQCRICMRNFSQSGDLSEHIRTHTGEKPFACDICGRKFATSGHLTTHTKIHTGSQKPFQCRICMRNFSDSSHLTTHIRTHTGEKPFACDICGRKFARSSHLTTHTKIHTGSQKPFQCRICMRNFSDRSDLTRHIRTHTGEKPFACDICGRKFADRSDLTRHTKIHTGSQKPFQCRICMRNFSRSDTLTRHIRTHTGLRGSNSGDPRRHSLGGSRKPDHVIQILDLFEKPLLLSIVSKP

SEQ ID NO: 53 - protein, 300 aa, Artificial Sequence
  Description of Artificial Sequence Synthetic polypeptide
MAAMAERPFQCRICMRNFSRSSDLTRHIRTHTGEKPFACDICGRKFARSDTLTRHTKIHTGSQKPFQCRICMRNFSQSGDLSEHIRTHTGEKPFACDICGRKFATSGHLTTHTKIHTGSQKPFQCRICMRNFSDSSHLTTHIRTHTGEKPFACDICGRKFARSSHLTTHTKIHTGSQKPFQCRICMRNFSDRSDLTRHIRTHTGEKPFACDICGRKFADRSDLTRHTKIHTGSQKPFQCRICMRNFSRSDTLTRHIRTHTGLRGSNSGDPRRHSLGGSRKPDIILVNDNISLILILVAKP

SEQ ID NO: 54 - protein, 298 aa, Artificial Sequence
  Description of Artificial Sequence Synthetic polypeptide
MAAMAERPFQCRICMRNFSRSSDLTRHIRTHTGEKPFACDICGRKFARSDTLTRHTKIHTGSQKPFQCRICMRNFSQSGDLSEHIRTHTGEKPFACDICGRKFATSGHLTTHTKIHTGSQKPFQCRICMRNFSDSSHLTTHIRTHTGEKPFACDICGRKFARSSHLTTHTKIHTGSQKPFQCRICMRNFSDRSDLTRHIRTHTGEKPFACDICGRKFADRSDLTRHTKIHTGSQKPFQCRICMRNFSRSDTLTRHIRTHTGLRGSNSGDPRRHSLGGSRKPDLIAYKNDDLLVIVAKP

SEQ ID NO: 55 - protein, 297 aa, Artificial Sequence
  Description of Artificial Sequence Synthetic polypeptide
MAAMAERPFQCRICMRNFSRSSDLTRHIRTHTGEKPFACDICGRKFARSDTLTRHTKIHTGSQKPFQCRICMRNFSQSGDLSEHIRTHTGEKPFACDICGRKFATSGHLTTHTKIHTGSQKPFQCRICMRNFSDSSHLTTHIRTHTGEKPFACDICGRKFARSSHLTTHTKIHTGSQKPFQCRICMRNFSDRSDLTRHIRTHTGEKPFACDICGRKFADRSDLTRHTKIHTGSQKPFQCRICMRNFSRSDTLTRHIRTHTGLRGSNSGDPRRHSLGKIVPALIAYKNFDLLVIELKP

SEQ ID NO: 56 - protein, 298 aa, Artificial Sequence
  Description of Artificial Sequence Synthetic polypeptide
MAAMAERPFQCRICMRNFSRSSDLTRHIRTHTGEKPFACDICGRKFARSDTLTRHTKIHTGSQKPFQCRICMRNFSQSGDLSEHIRTHTGEKPFACDICGRKFATSGHLTTHTKIHTGSQKPFQCRICMRNFSDSSHLTTHIRTHTGEKPFACDICGRKFARSSHLTTHTKIHTGSQKPFQCRICMRNFSDRSDLTRHIRTHTGEKPFACDICGRKFADRSDLTRHTKIHTGSQKPFQCRICMRNFSRSDTLTRHIRTHTGLRGSNSGDPRRHSLGGSNKPALIAYKNFDLLVIELKP

SEQ ID NO: 57 - protein, 297 aa, Artificial Sequence
  Description of Artificial Sequence Synthetic polypeptide
MAAMAERPFQCRICMRNFSRSSDLTRHIRTHTGEKPFACDICGRKFARSDTLTRHTKIHTGSQKPFQCRICMRNFSQSGDLSEHIRTHTGEKPFACDICGRKFATSGHLTTHTKIHTGSQKPFQCRICMRNFSDSSHLTTHIRTHTGEKPFACDICGRKFARSSHLTTHTKIHTGSQKPFQCRICMRNFSDRSDLTRHIRTHTGEKPFACDICGRKFADRSDLTRHTKIHTGSQKPFQCRICMRNFSRSDTLTRHIRTHTGLRGSNSGDPRRHSLGTKRPALIAYKNFDLLVIELKP

SEQ ID NO: 58 - protein, 299 aa, Artificial Sequence
  Description of Artificial Sequence Synthetic polypeptide
MAAMAERPFQCRICMRNFSRSSDLTRHIRTHTGEKPFACDICGRKFARSDTLTRHTKIHTGSQKPFQCRICMRNFSQSGDLSEHIRTHTGEKPFACDICGRKFATSGHLTTHTKIHTGSQKPFQCRICMRNFSDSSHLTTHIRTHTGEKPFACDICGRKFARSSHLTTHTKIHTGSQKPFQCRICMRNFSDRSDLTRHIRTHTGEKPFACDICGRKFADRSDLTRHTKIHTGSQKPFQCRICMRNFSRSDTLTRHIRTHTGLRGSNSGDPRRHSLGGETKRPALIAYKNFDLLVIELKP

SEQ ID NO: 59 - protein, 297 aa, Artificial Sequence
  Description of Artificial Sequence Synthetic polypeptide
MAAMAERPFQCRICMRNFSRSSDLTRHIRTHTGEKPFACDICGRKFARSDTLTRHTKIHTGSQKPFQCRICMRNFSQSGDLSEHIRTHTGEKPFACDICGRKFATSGHLTTHTKIHTGSQKPFQCRICMRNFSDSSHLTTHIRTHTGEKPFACDICGRKFARSSHLTTHTKIHTGSQKPFQCRICMRNFSDRSDLTRHIRTHTGEKPFACDICGRKFADRSDLTRHTKIHTGSQKPFQCRICMRNFSRSDTLTRHIRTHTGLRGSNSGDPRRHSLGGKRPALIAYKNFDLLVIELKP

SEQ ID NO: 60 - protein, 299 aa, Artificial Sequence
  Description of Artificial Sequence Synthetic polypeptide
MAAMAERPFQCRICMRNFSRSSDLTRHIRTHTGEKPFACDICGRKFARSDTLTRHTKIHTGSQKPFQCRICMRNFSQSGDLSEHIRTHTGEKPFACDICGRKFATSGHLTTHTKIHTGSQKPFQCRICMRNFSDSSHLTTHIRTHTGEKPFACDICGRKFARSSHLTTHTKIHTGSQKPFQCRICMRNFSDRSDLTRHIRTHTGEKPFACDICGRKFADRSDLTRHTKIHTGSQKPFQCRICMRNFSRSDTLTRHIRTHTGLRGSNSGDPRRHSLGREDERPALIAYKNFDLLVIELKP

SEQ ID NO: 61 - RNA, 36 nt, Artificial Sequence
  Description of Artificial Sequence Synthetic oligonucleotide
gagcuggacggggacguaaaguuuuagagcuaugcu

SEQ ID NO: 62 - DNA, 24 bp, Unknown
  Description of Unknown EGFP2 target sequence
ggagctggacggggacgtaaacgg

SEQ ID NO: 63 - protein, 1410 aa, Artificial Sequence
  Description of Artificial Sequence Synthetic polypeptide
DKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDLRQKDAARGSNSGDPRRHSLGGSRKPDLIAYKNFDLLVIVLKP

SEQ ID NO: 64 - DNA, 4233 bp, Artificial Sequence
  Description of Artificial Sequence Synthetic polynucleotide
gacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgccatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacctgagacagaaggacgccgcccggggatccaattctggtgatcctcggagacacagtctgggcggttctcgtaaacccgatctgattgcctataaaaactttgatctgctggtcattgttcttaagccttga

SEQ ID NO: 65 - protein, 10 aa, Artificial Sequence
  Description of Artificial Sequence Synthetic peptide
LRQKDAARGS

SEQ ID NO: 66 - DNA, 4123 bp, Artificial Sequence
  Description of Artificial Sequence Synthetic polynucleotide
gactcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttatggactacaaagaccatgacggtgattataaagatcatgacatcgattacaaggatgacgatgacaagatggcccccaagaagaagaggaaggtgggcattcacggggtaccggcggcgatggccgagcggcccttccagtgcaggatctgtatgcgcaacttttctcgttcttctgctcttactcgtcacatcagaacccatacaggcgaaaagcctttcgcctgcgacatttgtgggagaaaatttgctcgttctgatactcttactcgtcataccaagatccacaccggctctcagaaaccattccagtgccgcatttgtatgcggaatttttccgatcgttctaatcttactcgtcatatccgcactcacaccggagagaagccctttgcttgcgacatttgtggcaggaaatttgctcgttctgataatcttactcgtcacactaagatccatactgggtcacagaaacctttccagtgccggatttgtatgagaaactttagccgttctgatcatcttactcgtcacatcagaacacatactgggctgagaggatccggcggcggcggcggctccggcggcggcggcggctccggcggcggcggcggctccggcggcggcggcggctccggcggcggcggcggctccggcggcggcggcggctccatggccgagcggcccttccagtgcaggatctgtatgcgcaacttttccgatcgttctaatcttactcgtcacatcagaacccatacaggcgaaaagcctttcgcctgcgacatttgtgggcggaaatttgctcgttctgatcatcttactcgtcacacaaagatccatactggcagccagaaaccattccagtgcaggatttgcatgagaaacttttccgatcgttctaatcttactcgtcacatccgcactcataccggagagaagccctttgcttgcgacatttgtggccggaaatttgctcgttctgattctctttctgaacatacaaagatccatactgggtctcagaaacctttccagtgcaggatttgtatgagaaatttttcccgttcttctaatcttactcgtcacatcagaacacatactggggagaagccctttgcatgcgacatttgtggacggaaatttgctcgttctgattctcttactcgtcataccaagattcactgagcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttctt

SEQ ID NO: 67 - DNA, 1185 bp, Artificial Sequence
  Description of Artificial Sequence Synthetic polynucleotide
atggactacaaagaccatgacggtgattataaagatcatgacatcgattacaaggatgacgatgacaagatggcccccaagaagaagaggaaggtgggcattcacggggtaccggcggcgatggccgagcggcccttccagtgcaggatctgtatgcgcaacttttctcgttcttctgctcttactcgtcacatcagaacccatacaggcgaaaagcctttcgcctgcgacatttgtgggagaaaatttgctcgttctgatactcttactcgtcataccaagatccacaccggctctcagaaaccattccagtgccgcatttgtatgcggaatttttccgatcgttctaatcttactcgtcatatccgcactcacaccggagagaagccctttgcttgcgacatttgtggcaggaaatttgctcgttctgataatcttactcgtcacactaagatccatactgggtcacagaaacctttccagtgccggatttgtatgagaaactttagccgttctgatcatcttactcgtcacatcagaacacatactgggctgagaggatccggcggcggcggcggctccggcggcggcggcggctccggcggcggcggcggctccggcggcggcggcggctccggcggcggcggcggctccggcggcggcggcggctccatggccgagcggcccttccagtgcaggatctgtatgcgcaacttttccgatcgttctaatcttactcgtcacatcagaacccatacaggcgaaaagcctttcgcctgcgacatttgtgggcggaaatttgctcgttctgatcatcttactcgtcacacaaagatccatactggcagccagaaaccattccagtgcaggatttgcatgagaaacttttccgatcgttctaatcttactcgtcacatccgcactcataccggagagaagccctttgcttgcgacatttgtggccggaaatttgctcgttctgattctctttctgaacatacaaagatccatactgggtctcagaaacctttccagtgcaggatttgtatgagaaatttttcccgttcttctaatcttactcgtcacatcagaacacatactggggagaagccctttgcatgcgacatttgtggacggaaatttgctcgttctgattctcttactcgtcataccaagattcactga

SEQ ID NO: 68 - protein, 394 aa, Artificial Sequence
  Description of Artificial Sequence Synthetic polypeptide
MDYKDHDGDYKDHDIDYKDDDDKMAPKKKRKVGIHGVPAAMAERPFQCRICMRNFSRSSALTRHIRTHTGEKPFACDICGRKFARSDTLTRHTKIHTGSQKPFQCRICMRNFSDRSNLTRHIRTHTGEKPFACDICGRKFARSDNLTRHTKIHTGSQKPFQCRICMRNFSRSDHLTRHIRTHTGLRGSGGGGGSGGGGGSGGGGGSGGGGGSGGGGGSGGGGGSMAERPFQCRICMRNFSDRSNLTRHIRTHTGEKPFACDICGRKFARSDHLTRHTKIHTGSQKPFQCRICMRNFSDRSNLTRHIRTHTGEKPFACDICGRKFARSDSLSEHTKIHTGSQKPFQCRICMRNFSRSSNLTRHIRTHTGEKPFACDICGRKFARSDSLTRHTKIH

SEQ ID NO: 69 - protein, 36 aa, Artificial Sequence; identical to SEQ ID NO: 14
  Description of Artificial Sequence Synthetic polypeptide

SEQ ID NO: 70 - DNA, 142 bp, Artificial Sequence
  Description of Artificial Sequence Synthetic polynucleotide
gtggcatcgccctcgccctcgccggacacgctgaacttgtggccgtttacgtccccgtccagctccacgaggatggggacgacgccggtgaacagctcctcgcccttgctcaccataagcttaagtttaaacgctagccagc

SEQ ID NO: 71 - DNA, 3604 bp, Artificial Sequence
  Description of Artificial Sequence Synthetic polynucleotide
gactcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttatggactacaaagaccatgacggtgattataaagatcatgacatcgattacaaggatgacgatgacaagatggcccccaagaagaagaggaaggtgggcattcacggggtaccggcggcgatggccgagcggcccttccagtgcaggatctgtatgcgcaacttttctcgttcttctgctcttactcgtcacatcagaacccatacaggcgaaaagcctttcgcctgcgacatttgtgggagaaaatttgctcgttctgatactcttactcgtcataccaagatccacaccggctctcagaaaccattccagtgccgcatttgtatgcggaatttttccgatcgttctaatcttactcgtcatatccgcactcacaccggagagaagccctttgcttgcgacatttgtggcaggaaatttgctcgttctgataatcttactcgtcacactaagatccatactgggtcacagaaacctttccagtgccggatttgtatgagaaactttagccgttctgatcatcttactcgtcacatcagaacacatactgggctgagaggatccaattctggtgatcctcggagacacagtctgggcggttctcgtaaacccgctctgattgcctataaaaactttgatctgctggtcattgaacttaagccttgagcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttctt

SEQ ID NO: 72 - DNA, 666 bp, Artificial Sequence
  Description of Artificial Sequence Synthetic polynucleotide
atggactacaaagaccatgacggtgattataaagatcatgacatcgattacaaggatgacgatgacaagatggcccccaagaagaagaggaaggtgggcattcacggggtaccggcggcgatggccgagcggcccttccagtgcaggatctgtatgcgcaacttttctcgttcttctgctcttactcgtcacatcagaacccatacaggcgaaaagcctttcgcctgcgacatttgtgggagaaaatttgctcgttctgatactcttactcgtcataccaagatccacaccggctctcagaaaccattccagtgccgcatttgtatgcggaatttttccgatcgttctaatcttactcgtcatatccgcactcacaccggagagaagccctttgcttgcgacatttgtggcaggaaatttgctcgttctgataatcttactcgtcacactaagatccatactgggtcacagaaacctttccagtgccggatttgtatgagaaactttagccgttctgatcatcttactcgtcacatcagaacacatactgggctgagaggatccaattctggtgatcctcggagacacagtctgggcggttctcgtaaacccgctctgattgcctataaaaactttgatctgctggtcattgaacttaagccttga

SEQ ID NO: 73 - protein, 221 aa, Artificial Sequence
  Description of Artificial Sequence Synthetic polypeptide
MDYKDHDGDYKDHDIDYKDDDDKMAPKKKRKVGIHGVPAAMAERPFQCRICMRNFSRSSALTRHIRTHTGEKPFACDICGRKFARSDTLTRHTKIHTGSQKPFQCRICMRNFSDRSNLTRHIRTHTGEKPFACDICGRKFARSDNLTRHTKIHTGSQKPFQCRICMRNFSRSDHLTRHIRTHTGLRGSNSGDPRRHSLGGSRKPALIAYKNFDLLVIELKP

SEQ ID NO: 74 - DNA, 666 bp, Artificial Sequence
  Description of Artificial Sequence Synthetic polynucleotide
atggactacaaagaccatgacggtgattataaagatcatgacatcgattacaaggatgacgatgacaagatggcccccaagaagaagaggaaggtgggcattcacggggtaccggcggcgatggccgagcggcccttccagtgcaggatctgtatgcgcaacttttctcgttcttctgctcttactcgtcacatcagaacccatacaggcgaaaagcctttcgcctgcgacatttgtgggagaaaatttgctcgttctgatactcttactcgtcataccaagatccacaccggctctcagaaaccattccagtgccgcatttgtatgcggaatttttccgatcgttctaatcttactcgtcatatccgcactcacaccggagagaagccctttgcttgcgacatttgtggcaggaaatttgctcgttctgataatcttactcgtcacactaagatccatactgggtcacagaaacctttccagtgccggatttgtatgagaaactttagccgttctgatcatcttactcgtcacatcagaacacatactgggctgagaggatccaattctggtgatcctcggagacacagtctgggcggttctcgtaaacccgatctgattgcctataaaaactttgatctgctggtcattgttcttaagccttga

SEQ ID NO: 75 - protein, 221 aa, Artificial Sequence
  Description of Artificial Sequence Synthetic polypeptide
MDYKDHDGDYKDHDIDYKDDDDKMAPKKKRKVGIHGVPAAMAERPFQCRICMRNFSRSSALTRHIRTHTGEKPFACDICGRKFARSDTLTRHTKIHTGSQKPFQCRICMRNFSDRSNLTRHIRTHTGEKPFACDICGRKFARSDNLTRHTKIHTGSQKPFQCRICMRNFSRSDHLTRHIRTHTGLRGSNSGDPRRHSLGGSRKPDLIAYKNFDLLVIVLKP

SEQ ID NO: 76 - DNA, 23 bp, Artificial Sequence
  Description of Artificial Sequence Synthetic primer
ccatatatggagttccgcgttac

SEQ ID NO: 77 - DNA, 20 bp, Artificial Sequence
  Description of Artificial Sequence Synthetic primer
gcttgtcggccatgatatag

SEQ ID NO: 78 - DNA, 19 bp, Artificial Sequence
  Description of Artificial Sequence Synthetic primer
ccaagctggctagcgttta

SEQ ID NO: 79 - DNA, 20 bp, Artificial Sequence
  Description of Artificial Sequence Synthetic primer
gaacttcagggtcagcttgc

SEQ ID NO: 80 - DNA, 22 bp, Artificial Sequence
  Description of Artificial Sequence Synthetic primer
ggtcatcggcatcgcggaggag

SEQ ID NO: 81 - protein, 33 aa, Artificial Sequence
  Description of Artificial Sequence Synthetic polypeptide
NSGDPRRHSLGGSRKPDLIAYKNFDLLVIELKP

SEQ ID NO: 82 - DNA, 16 bp, Artificial Sequence
  Description of Artificial Sequence Synthetic primer
ctccgcgatgccgatg

SEQ ID NO: 83 - DNA, 15 bp, Artificial Sequence
  Description of Artificial Sequence Synthetic primer
cgcggccctgttcca

SEQ ID NO: 84 - protein, 33 aa, Artificial Sequence
  Description of Artificial Sequence Synthetic polypeptide
NSGDPRRHSLGGSRKPALIAYKNFDLLVIELKP

SEQ ID NO: 85 - DNA, 102 bp, Artificial Sequence
  Description of Artificial Sequence Synthetic polynucleotide
aattctggtgatcctcggagacacagtctgggcggttctcgtaaacccgctctgattgcctataaaaactttgatctgctggtcattgaacttaagccttga

SEQ ID NO: 86 - protein, 170 aa, Artificial Sequence
  Description of Artificial Sequence Synthetic polypeptide
MAERPFQCRICMRNFSDRSNLTRHIRTHTGEKPFACDICGRKFARSDHLTRHTKIHTGSQKPFQCRICMRNFSDRSNLTRHIRTHTGEKPFACDICGRKFARSDSLSEHTKIHTGSQKPFQCRICMRNFSRSSNLTRHIRTHTGEKPFACDICGRKFARSDSLTRHTKIH

SEQ ID NO: 87 - DNA, 897 bp, Artificial Sequence
  Description of Artificial Sequence Synthetic polynucleotide
atggcggcgatggccgagcggcccttccagtgcaggatctgtatgcgcaacttttctcggtcctccgacctgacccggcacatcagaacccatacaggcgaaaagcctttcgcctgcgacatttgtgggagaaaatttgctcggtccgacaccctgacccggcataccaagatccacaccggctctcagaaaccattccagtgccgcatttgtatgcggaatttttcccagtccggcgacctgtccgagcatatccgcactcacaccggagagaagccctttgcttgcgacatttgtggcaggaaatttgctacctccggccacctgaccacccacactaagatccatactgggtcacagaaacctttccagtgccggatttgtatgagaaactttagcgactcctcccacctgaccacccacatcagaacccatacaggcgaaaagcctttcgcctgcgacatttgtgggagaaaatttgctcggtcctcccacctgaccacccataccaagatccacaccggctctcagaaaccattccagtgccgcatttgtatgcggaatttttccgaccggtccgacctgacccggcatatccgcactcacaccggagagaagccctttgcttgcgacatttgtggcaggaaatttgctgaccggtccgacctgacccggcacactaagatccatactgggtcacagaaacctttccagtgccggatttgtatgagaaactttagccggtccgacaccctgacccggcacatcagaacacatactgggctgagaggatccaattctggtgatcctcggagacacagtctgggcggttctcgtaaacccgatctgattgcctataaaaactttgatctgctggtcattgttcttaagccttga

SEQ ID NO: 88 - protein, 298 aa, Artificial Sequence
  Description of Artificial Sequence Synthetic polypeptide
MAAMAERPFQCRICMRNFSRSSDLTRHIRTHTGEKPFACDICGRKFARSDTLTRHTKIHTGSQKPFQCRICMRNFSQSGDLSEHIRTHTGEKPFACDICGRKFATSGHLTTHTKIHTGSQKPFQCRICMRNFSDSSHLTTHIRTHTGEKPFACDICGRKFARSSHLTTHTKIHTGSQKPFQCRICMRNFSDRSDLTRHIRTHTGEKPFACDICGRKFADRSDLTRHTKIHTGSQKPFQCRICMRNFSRSDTLTRHIRTHTGLRGSNSGDPRRHSLGGSRKPDLIAYKNFDLLVIVLKP

SEQ ID NO: 89 - DNA, 1068 bp, Artificial Sequence
  Description of Artificial Sequence Synthetic polynucleotide
atggcggcgatggccgagcggcccttccagtgcaggatctgtatgcgcaacttttctgaccggtcccacctgacccggcacatcagaacccatacaggcgaaaagcctttcgcctgcgacatttgtgggagaaaatttgctcggtccgacaacctgacccggcataccaagatccacaccggctctcagaaaccattccagtgccgcatttgtatgcggaatttttccgactcctcccacctgtccgagcatatccgcactcacaccggagagaagccctttgcttgcgacatttgtggcaggaaatttgctgaccggtccgacctgacccggcacactaagatccatactgggtcacagaaacctttccagtgccggatttgtatgagaaactttagccggtccgaccacctgacccggcacatcagaacccatacaggcgaaaagcctttcgcctgcgacatttgtgggagaaaatttgctgaccggtccgacctgacccggcataccaagatccacaccggctctcagaaaccattccagtgccgcatttgtatgcggaatttttcccggtccgacaacctgtccgagcatatccgcactcacaccggagagaagccctttgcttgcgacatttgtggcaggaaatttgctgagtcctccaacctgaccacccataccaagatccacaccggctctcagaaaccattccagtgccgcatttgtatgcggaatttttcccggtcctcctccctgacccggcatatccgcactcacaccggagagaagccctttgcttgcgacatttgtggcaggaaatttgctcagtcctccgacctgacccggcacactaagatccatactgggtcacagaaacctttccagtgccggatttgtatgagaaactttagccggtccgactccctgtccgagcacatcagaacacatactgggctgagaggatccaattctggtgatcctcggagacacagtctgggcggttctcgtaaacccgatctgattgcctataaaaactttgatctgctggtcattgttcttaagccttga

SEQ ID NO: 90 - protein, 355 aa, Artificial Sequence
  Description of Artificial Sequence Synthetic polypeptide
MAAMAERPFQCRICMRNFSDRSHLTRHIRTHTGEKPFACDICGRKFARSDNLTRHTKIHTGSQKPFQCRICMRNFSDSSHLSEHIRTHTGEKPFACDICGRKFADRSDLTRHTKIHTGSQKPFQCRICMRNFSRSDHLTRHIRTHTGEKPFACDICGRKFADRSDLTRHTKIHTGSQKPFQCRICMRNFSRSDNLSEHIRTHTGEKPFACDICGRKFAESSNLTTHTKIHTGSQKPFQCRICMRNFSRSSSLTRHIRTHTGEKPFACDICGRKFAQSSDLTRHTKIHTGSQKPFQCRICMRNFSRSDSLSEHIRTHTGLRGSNSGDPRRHSLGGSRKPDLIAYKNFDLLVIVLKP

SEQ ID NO: 91 - DNA, 69 bp, Artificial Sequence
  Description of Artificial Sequence Synthetic oligonucleotide
  modified_base (34)..(54): a, c, t, g, unknown or other
ttccagtgccggatctgcatgcggaacttctccnnnnnnnnnnnnnnnnnnnnncacatccggacccac

SEQ ID NO: 92 - DNA, 69 bp, Artificial Sequence
  Description of Artificial Sequence Synthetic oligonucleotide
  modified_base (34)..(54): a, c, t, g, unknown or other
tttgcgtgcgatatttgcggccgtaaatttgcgnnnnnnnnnnnnnnnnnnnnncataccaaaattcat

SEQ ID NO: 93 - DNA, 417 bp, Artificial Sequence
  Description of Artificial Sequence Synthetic polynucleotide
ttccagtgcaggatctgtatgcgcaacttttctcgttcttctgctcttactcgtcacatcagaacccatacaggcgaaaagcctttcgcctgcgacatttgtgggagaaaatttgctcgttctgatactcttactcgtcataccaagatccacaccggctctcagaaaccattccagtgccgcatttgtatgcggaatttttccgatcgttctaatcttactcgtcatatccgcactcacaccggagagaagccctttgcttgcgacatttgtggcaggaaatttgctcgttctgataatcttactcgtcacactaagatccatactgggtcacagaaacctttccagtgccggatttgtatgagaaactttagccgttctgatcatcttactcgtcacatcagaacacatactggg

SEQ ID NO: 94 - DNA, 495 bp, Artificial Sequence
  Description of Artificial Sequence Synthetic polynucleotide
ttccagtgcaggatctgtatgcgcaacttttccgatcgttctaatcttactcgtcacatcagaacccatacaggcgaaaagcctttcgcctgcgacatttgtgggcggaaatttgctcgttctgatcatcttactcgtcacacaaagatccatactggcagccagaaaccattccagtgcaggatttgcatgagaaacttttccgatcgttctaatcttactcgtcacatccgcactcataccggagagaagccctttgcttgcgacatttgtggccggaaatttgctcgttctgattctctttctgaacatacaaagatccatactgggtctcagaaacctttccagtgcaggatttgtatgagaaatttttcccgttcttctaatcttactcgtcacatcagaacacatactggggagaagccctttgcatgcgacatttgtggacggaaatttgctcgttctgattctcttactcgtcataccaagattcac

SEQ ID NO: 95 - DNA, 759 bp, Artificial Sequence
  Description of Artificial Sequence Synthetic polynucleotide
ttccagtgcaggatctgtatgcgcaacttttctcggtcctccgacctgacccggcacatcagaacccatacaggcgaaaagcctttcgcctgcgacatttgtgggagaaaatttgctcggtccgacaccctgacccggcataccaagatccacaccggctctcagaaaccattccagtgccgcatttgtatgcggaatttttcccagtccggcgacctgtccgagcatatccgcactcacaccggagagaagccctttgcttgcgacatttgtggcaggaaatttgctacctccggccacctgaccacccacactaagatccatactgggtcacagaaacctttccagtgccggatttgtatgagaaactttagcgactcctcccacctgaccacccacatcagaacccatacaggcgaaaagcctttcgcctgcgacatttgtgggagaaaatttgctcggtcctcccacctgaccacccataccaagatccacaccggctctcagaaaccattccagtgccgcatttgtatgcggaatttttccgaccggtccgacctgacccggcatatccgcactcacaccggagagaagccctttgcttgcgacatttgtggcaggaaatttgctgaccggtccgacctgacccggcacactaagatccatactgggtcacagaaacctttccagtgccggatttgtatgagaaactttagccggtccgacaccctgacccggcacatcagaacacatactggg

SEQ ID NO: 96 - DNA, 930 bp, Artificial Sequence
  Description of Artificial Sequence Synthetic polynucleotide
ttccagtgcaggatctgtatgcgcaacttttctgaccggtcccacctgacccggcacatcagaacccatacaggcgaaaagcctttcgcctgcgacatttgtgggagaaaatttgctcggtccgacaacctgacccggcataccaagatccacaccggctctcagaaaccattccagtgccgcatttgtatgcggaatttttccgactcctcccacctgtccgagcatatccgcactcacaccggagagaagccctttgcttgcgacatttgtggcaggaaatttgctgaccggtccgacctgacccggcacactaagatccatactgggtcacagaaacctttccagtgccggatttgtatgagaaactttagccggtccgaccacctgacccggcacatcagaacccatacaggcgaaaagcctttcgcctgcgacatttgtgggagaaaatttgctgaccggtccgacctgacccggcataccaagatccacaccggctctcagaaaccattccagtgccgcatttgtatgcggaatttttcccggtccgacaacctgtccgagcatatccgcactcacaccggagagaagccctttgcttgcgacatttgtggcaggaaatttgctgagtcctccaacctgaccacccataccaagatccacaccggctctcagaaaccattccagtgccgcatttgtatgcggaatttttcccggtcctcctccctgacccggcatatccgcactcacaccggagagaagccctttgcttgcgacatttgtggcaggaaatttgctcagtcctccgacctgacccggcacactaagatccatactgggtcacagaaacctttccagtgccggatttgtatgagaaactttagccggtccgactccctgtccgagcacatcagaacacatactggg

SEQ ID NO: 97 - DNA, 4101 bp, Artificial Sequence
  Description of Artificial Sequence Synthetic polynucleotide
gacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgccatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgac

SEQ ID NO: 98 - DNA, 12 bp, Artificial Sequence
  Description of Artificial Sequence Synthetic oligonucleotide
ctgagaggatcc

SEQ ID NO: 99 - DNA, 30 bp, Artificial Sequence
  Description of Artificial Sequence Synthetic oligonucleotide
ctgagacagaaggacgccgcccggggatcc

SEQ ID NO: 100 - DNA, 108 bp, Artificial Sequence
  Description of Artificial Sequence Synthetic polynucleotide
ggcggcggcggcggctccggcggcggcggcggctccggcggcggcggcggctccggcggcggcggcggctccggcggcggcggcggctccggcggcggcggcggctcc

SEQ ID NO: 101 - protein, 33 aa, Artificial Sequence
  Description of Artificial Sequence Synthetic polypeptide
NSGDPRRHSLGGSRKPDLIAYKNFDLLVIQLKP

SEQ ID NO: 102 - protein, 33 aa, Artificial Sequence
  Description of Artificial Sequence Synthetic polypeptide
NSGDPRRHSLGGSRKPDLIAYKNFDLLVINLKP

SEQ ID NO: 103 - protein, 33 aa, Artificial Sequence
  Description of Artificial Sequence Synthetic polypeptide
NSGDPRRHSLGGSRKPDLIAYKNFDLLVISLKP

SEQ ID NO: 104 - protein, 33 aa, Artificial Sequence
  Description of Artificial Sequence Synthetic polypeptide
NSGDPRRHSLGGSRKPDLIAYKNFDLLVITLKP

SEQ ID NO: 105 - protein, 33 aa, Artificial Sequence
  Description of Artificial Sequence Synthetic polypeptide
NSGDPRRHSLGGSRKPDLIAYKNFDLLVIALKP

SEQ ID NO: 106 - protein, 33 aa, Artificial Sequence; identical to SEQ ID NO: 19
  Description of Artificial Sequence Synthetic polypeptide

SEQ ID NO: 107 - protein, 33 aa, Artificial Sequence
  Description of Artificial Sequence Synthetic polypeptide
NSGDPRRHSLGGSRKPDLIAYKNFDLLVILLKP

SEQ ID NO: 108 - protein, 33 aa, Artificial Sequence
  Description of Artificial Sequence Synthetic polypeptide
NSGDPRRHSLGGSRKPDLIAYKNFDLLVIILKP

SEQ ID NO: 109 - protein, 33 aa, Artificial Sequence
  Description of Artificial Sequence Synthetic polypeptide
NSGDPRRHSLGGSRKPDLIAYKNFDLLVIHLKP

SEQ ID NO: 110 - protein, 33 aa, Artificial Sequence
  Description of Artificial Sequence Synthetic polypeptide
NSGDPRRHSLGGSRKPDLIAYKNFDLLVIRLKP

SEQ ID NO: 111 - protein, 33 aa, Artificial Sequence
  Description of Artificial Sequence Synthetic polypeptide
NSGDPRRHSLGGSRKPDLIAYKNFDLLVIKLKP

SEQ ID NO: 112 - protein, 33 aa, Artificial Sequence
  Description of Artificial Sequence Synthetic polypeptide
NSGDPRRHSLGGSRKPDLIAYKNFDLLVIMLKP

SEQ ID NO: 113 - protein, 33 aa, Artificial Sequence
  Description of Artificial Sequence Synthetic polypeptide
NSGDPRRHSLGGSRKPNLIAYKNFDLLVIELKP

SEQ ID NO: 114 - protein, 33 aa, Artificial Sequence; identical to SEQ ID NO: 84
  Description of Artificial Sequence Synthetic polypeptide

SEQ ID NO: 115 - protein, 37 aa, Artificial Sequence
  Description of Artificial Sequence Synthetic polypeptide
NSGDPRRHSLGGSRKPDGAIYTVGSPIDYGVIVVTKP

SEQ ID NO: 116 - protein, 36 aa, Artificial Sequence
  Description of Artificial Sequence Synthetic polypeptide
NSGDPRRHSLGGSRKPDFTLYKPSEPNKKIAIVIKP

SEQ ID NO: 117 - protein, 33 aa, Artificial Sequence
  Description of Artificial Sequence Synthetic polypeptide
NSGDPRRHSLGGSRKPDGLLWDDDCAIILVSKP

SEQ ID NO: 118 - protein, 37 aa, Artificial Sequence
  Description of Artificial Sequence Synthetic polypeptide
NSGDPRRHSLGGSRKPDHIYQLVYNSTDTLLLIVSKP

SEQ ID NO: 119 - protein, 38 aa, Artificial Sequence
  Description of Artificial Sequence Synthetic polypeptide
NSGDPRRHSLGGSRKPDHIYIFNDDNNTKNGLIIVSKP

SEQ ID NO: 120 - protein, 38 aa, Artificial Sequence
  Description of Artificial Sequence Synthetic polypeptide
NSGDPRRHSLGGSRKPDHVIQILDLFEKPLLLSIVSKP

SEQ ID NO: 121 - protein, 35 aa, Artificial Sequence
  Description of Artificial Sequence Synthetic polypeptide
NSGDPRRHSLGGSRKPDIILVNDNISLILILVAKP

SEQ ID NO: 122 - protein, 33 aa, Artificial Sequence
  Description of Artificial Sequence Synthetic polypeptide
NSGDPRRHSLGGSRKPDLIAYKNDDLLVIVAKP

SEQ ID NO: 123 - protein, 32 aa, Artificial Sequence
  Description of Artificial Sequence Synthetic polypeptide
NSGDPRRHSLGKIVPALIAYKNFDLLVIELKP

SEQ ID NO: 124 - protein, 33 aa, Artificial Sequence
  Description of Artificial Sequence Synthetic polypeptide
NSGDPRRHSLGGSNKPALIAYKNFDLLVIELKP

SEQ ID NO: 125 - protein, 32 aa, Artificial Sequence
  Description of Artificial Sequence Synthetic polypeptide
NSGDPRRHSLGTKRPALIAYKNFDLLVIELKP

SEQ ID NO: 126 - protein, 34 aa, Artificial Sequence
  Description of Artificial Sequence Synthetic polypeptide
NSGDPRRHSLGGETKRPALIAYKNFDLLVIELKP

SEQ ID NO: 127 - protein, 32 aa, Artificial Sequence
  Description of Artificial Sequence Synthetic polypeptide
NSGDPRRHSLGGKRPALIAYKNFDLLVIELKP

SEQ ID NO: 128 - protein, 34 aa, Artificial Sequence
  Description of Artificial Sequence Synthetic polypeptide
NSGDPRRHSLGREDERPALIAYKNFDLLVIELKP

SEQ ID NO: 129 - DNA, 99 bp, Artificial Sequence
  Description of Artificial Sequence Synthetic oligonucleotide
aattctggtgatcctcggagacacagtctgggcggttctcgtaaacccgatctgattgcctataaaaactttgatctgctggtcattcaacttaagcct

SEQ ID NO: 130 - DNA, 99 bp, Artificial Sequence
  Description of Artificial Sequence Synthetic oligonucleotide
aattctggtgatcctcggagacacagtctgggcggttctcgtaaacccgatctgattgcctataaaaactttgatctgctggtcattaatcttaagcct

SEQ ID NO: 131 - DNA, 99 bp, Artificial Sequence
  Description of Artificial Sequence Synthetic oligonucleotide
aattctggtgatcctcggagacacagtctgggcggttctcgtaaacccgatctgattgcctataaaaactttgatctgctggtcatttctcttaagcct

SEQ ID NO: 132 - DNA, 99 bp, Artificial Sequence
  Description of Artificial Sequence Synthetic oligonucleotide
aattctggtgatcctcggagacacagtctgggcggttctcgtaaacccgatctgattgcctataaaaactttgatctgctggtcattactcttaagcct

SEQ ID NO: 133 - DNA, 99 bp, Artificial Sequence
  Description of Artificial Sequence Synthetic oligonucleotide
aattctggtgatcctcggagacacagtctgggcggttctcgtaaacccgatctgattgcctataaaaactttgatctgctggtcattgctcttaagcct

SEQ ID NO: 134 - DNA, 99 bp, Artificial Sequence
  Description of Artificial Sequence Synthetic oligonucleotide
aattctggtgatcctcggagacacagtctgggcggttctcgtaaacccgatctgattgcctataaaaactttgatctgctggtcattgttcttaagcct

SEQ ID NO: 135 - DNA, 99 bp, Artificial Sequence
  Description of Artificial Sequence Synthetic oligonucleotide
aattctggtgatcctcggagacacagtctgggcggttctcgtaaacccgatctgattgcctataaaaactttgatctgctggtcattcttcttaagcct

SEQ ID NO: 136 - DNA, 99 bp, Artificial Sequence
  Description of Artificial Sequence Synthetic oligonucleotide
aattctggtgatcctcggagacacagtctgggcggttctcgtaaacccgatctgattgcctataaaaactttgatctgctggtcattattcttaagcct

SEQ ID NO: 137 - DNA, 99 bp, Artificial Sequence
  Description of Artificial Sequence Synthetic oligonucleotide
aattctggtgatcctcggagacacagtctgggcggttctcgtaaacccgatctgattgcctataaaaactttgatctgctggtcattcatcttaagcct

SEQ ID NO: 138 - DNA, 99 bp, Artificial Sequence
  Description of Artificial Sequence Synthetic oligonucleotide
aattctggtgatcctcggagacacagtctgggcggttctcgtaaacccgatctgattgcctataaaaactttgatctgctggtcattcgtcttaagcct

SEQ ID NO: 139 - DNA, 99 bp, Artificial Sequence
  Description of Artificial Sequence Synthetic oligonucleotide
aattctggtgatcctcggagacacagtctgggcggttctcgtaaacccgatctgattgcctataaaaactttgatctgctggtcattaaacttaagcct

SEQ ID NO: 140 - DNA, 99 bp, Artificial Sequence
  Description of Artificial Sequence Synthetic oligonucleotide
aattctggtgatcctcggagacacagtctgggcggttctcgtaaacccgatctgattgcctataaaaactttgatctgctggtcattatgcttaagcct

SEQ ID NO: 141 - DNA, 99 bp, Artificial Sequence
  Description of Artificial Sequence Synthetic oligonucleotide
aattctggtgatcctcggagacacagtctgggcggttctcgtaaacccaatctgattgcctataaaaactttgatctgctggtcattgaacttaagcct

SEQ ID NO: 142 - DNA, 99 bp, Artificial Sequence
  Description of Artificial Sequence Synthetic oligonucleotide
aattctggtgatcctcggagacacagtctgggcggttctcgtaaacccgctctgattgcctataaaaactttgatctgctggtcattgaacttaagcct

SEQ ID NO: 143 - DNA, 111 bp, Artificial Sequence
  Description of Artificial Sequence Synthetic polynucleotide
aattctggtgatcctcggagacacagtctgggcggttctcgtaaacccgatggtgctatttatactgttggttctcctattgattatggtgttattgttgttactaaacct

SEQ ID NO: 144 - DNA, 108 bp, Artificial Sequence
  Description of Artificial Sequence Synthetic polynucleotide
aattctggtgatcctcggagacacagtctgggcggttctcgtaaacccgattttactctttataaaccttctgaacctaataaaaaaattgctattgttattaaacct

SEQ ID NO: 145 - DNA, 99 bp, Artificial Sequence
  Description of Artificial Sequence Synthetic oligonucleotide
aattctggtgatcctcggagacacagtctgggcggttctcgtaaacccgatggtcttctttgggatgatgattgtgctattattcttgtttctaaacct

SEQ ID NO: 146 - DNA, 111 bp, Artificial Sequence
  Description of Artificial Sequence Synthetic polynucleotide
aattctggtgatcctcggagacacagtctgggcggttctcgtaaacccgatcatatttatcaacttgtttataattctactgatactcttcttcttattgtttctaaacct

SEQ ID NO: 147 - DNA, 114 bp, Artificial Sequence
  Description of Artificial Sequence Synthetic polynucleotide
aattctggtgatcctcggagacacagtctgggcggttctcgtaaacccgatcatatttatatttttaatgatgataataatactaaaaatggtcttattattgtttctaaacct

SEQ ID NO: 148 - DNA, 114 bp, Artificial Sequence
  Description of Artificial Sequence Synthetic polynucleotide
aattctggtgatcctcggagacacagtctgggcggttctcgtaaacccgatcatgttattcaaattcttgatctttttgaaaaacctcttcttctttctattgtttctaaacct

SEQ ID NO: 149 - DNA, 105 bp, Artificial Sequence
  Description of Artificial Sequence Synthetic polynucleotide
aattctggtgatcctcggagacacagtctgggcggttctcgtaaacccgatattattcttgttaatgataatatttctcttattcttattcttgttgctaaacct

SEQ ID NO: 150 - DNA, 99 bp, Artificial Sequence
  Description of Artificial Sequence Synthetic oligonucleotide
aattctggtgatcctcggagacacagtctgggcggttctcgtaaacccgatcttattgcttataaaaatgatgatcttcttgttattgttgctaaacct

SEQ ID NO: 151 - DNA, 96 bp, Artificial Sequence
  Description of Artificial Sequence Synthetic oligonucleotide
aattctggtgatcctcggagacacagtctgggcaagatcgtgcccgctctgattgcctataaaaactttgatctgctggtcattgaacttaagcct

SEQ ID NO: 152 - DNA, 99 bp, Artificial Sequence
  Description of Artificial Sequence Synthetic oligonucleotide
aattctggtgatcctcggagacacagtctgggcggttctaacaaacccgctctgattgcctataaaaactttgatctgctggtcattgaacttaagcct

SEQ ID NO: 153 - DNA, 96 bp, Artificial Sequence
  Description of Artificial Sequence Synthetic oligonucleotide
aattctggtgatcctcggagacacagtctgggcaccaagcggcccgctctgattgcctataaaaactttgatctgctggtcattgaacttaagcct

SEQ ID NO: 154 - DNA, 102 bp, Artificial Sequence
  Description of Artificial Sequence Synthetic polynucleotide
aattctggtgatcctcggagacacagtctgggcggtgagaccaagcggcccgctctgattgcctataaaaactttgatctgctggtcattgaacttaagcct

SEQ ID NO: 155 - DNA, 96 bp, Artificial Sequence
  Description of Artificial Sequence Synthetic oligonucleotide
aattctggtgatcctcggagacacagtctgggcggtaagcggcccgctctgattgcctataaaaactttgatctgctggtcattgaacttaagcct

SEQ ID NO: 156 - DNA, 105 bp, Artificial Sequence
  Description of Artificial Sequence Synthetic polynucleotide
aattctggtgatcctcggagacacagtctgggccgggaggacgagcggcccgctctgattgcctataaaaactttgatctgctggtcattgaacttaagccttga

SEQ ID NO: 157 - DNA, 3647 bp, Homo sapiens
gggacagggggagccctataattggacaagtctgggatccttgagtcctactcagccccagcggaggtgaaggacgtccttccccaggagccggtgagaagcgcagtcgggggcacggggatgagctcaggggcctctagaaagagctgggaccctgggaacccctggcctccaggtagtctcaggagagctactcggggtcgggcttggggagaggaggagcgggggtgaggcaagcagcaggggactggacctgggaagggctgggcagcagagacgacccgacccgctagaaggtggggtggggagagcagctggactgggatgtaagccatagcaggactccacgagttgtcactatcatttatcgagcacctactgggtgtccccagtgtcctcagatctccataactggggagccaggggcagcgacacggtagctagccgtcgattggagaactttaaaatgaggactgaattagctcataaatggaacacggcgcttaactgtgaggttggagcttagaatgtgaagggagaatgaggaatgcgagactgggactgagatggaaccggcggtggggagggggtggggggatggaatttgaaccccgggagaggaagatggaattttctatggaggccgacctggggatggggagataagagaagaccaggagggagttaaatagggaatgggttgggggcggcttggtaaatgtgctgggattaggctgttgcagataatgcaacaaggcttggaaggctaacctggggtgaggccgggttggggccgggctgggggtgggaggagtcctcactggcggttgattgacagtttctccttccccagactggccaatcacaggcaggaagatgaaggttctgtgggctgcgttgctggtcacattcctggcaggtatgggggcggggcttgctcggttccccccgctcctccccctctcatcctcacctcaacctcctggccccattcaggcagaccctgggccccctcttctgaggcttctgtgctgcttcctggctctgaacagcgatttgacgctctctgggcctcggtttcccccatccttgagataggagttagaagttgttttgttgttgttgtttgttgttgttgttttgtttttttgagatgaagtctcgctctgtcgcccaggctggagtgcagtggcgggatctcggctcactgcaagctccgcctcccaggtccacgccattctcctgcctcagcctcccaagtagctgggactacaggcacatgccaccacacccgactaacttttttgtattttcagtagagacggggtttcaccatgttggccaggctggtctggaactcctgacctcaggtgatctgcccgtttcgatctcccaaagtgctgggattacaggcgtgagccaccgcacctggctgggagttagaggtttctaatgcattgcaggcagatagtgaataccagacacggggcagctgtgatctttattctccatcacccccacacagccctgcctggggcacacaaggacactcaatacatgcttttccgctgggcgcggtggctcacccctgtaatcccagcactttgggaggccaaggtgggaggatcacttgagcccaggagttcaacaccagcctgggcaacatagtgagaccctgtctctactaaaaatacaaaaattagccaggcatggtgccacacacctgtgctctcagctactcaggaggctgaggcaggaggatcgcttgagcccagaaggtcaaggttgcagtgaaccatgttcaggccgctgcactccagcctgggtgacagagcaagaccctgtttataaatacataatgctttccaagtgattaaaccgactcccccctcaccctgcccaccatggctccaaagaagcatttgtggagcaccttctgtgtgcccctaggtactagatgcctggacggggtcagaaggaccctgacccaccttgaacttgttccacacaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgaggtgagtgtccccatcctggcccttgaccctcctggtgggcggctatacctccccaggtccaggtttcattctgcccctgtcgctaagtcttggggggcctgggtctctgctggttctagcttcctcttcccatttctgactcctggctttagctctctggaattctctctctcagctttgtctctctctcttcccttctgactcagtctctcacactcgtcctggctctgtctctgtccttccctagctcttttatatagagacagagagatggggtctcactgtgttgcccaggctggtcttgaacttctgggctcaagcgatcctcccgcctcggcctcccaaagtgctgggattagaggcatgagccaccttgcccggcctcctagctccttcttcgtctctgcctctgccctctgcatctgctctctgcatctgtctctgtctccttctctcggcctctgccccgttccttctctccctcttgggtctctctggctcatccccatctcgcccgccccatcccagcccttctccccgcctcccactgtgcgacaccctcccgccctctcggccgcagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctgcgcaagctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactgaacgccgaagcctgcagccatgcgaccccacgccaccccgtgcctcctgcctccgcgcagcctgcagcgggagaccctgtccccgccccagccgtcctcctggggtggaccctagtttaataaagattcaccaagtttcacgcatc

SEQ ID NO: 158 - DNA, 5069 bp, Artificial Sequence
  Description of Artificial Sequence Synthetic polynucleotide
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccactagtccagtgtggtggaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggtccctttagggttccgatttagtgctttacggcacctcgaccccaaaaaacttgattagggtgatggttcacgtacctagaagttcctattccgaagttcctattctctagaaagtataggaacttccttggccaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagcacgtactacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 159 - DNA, 3679 bp, Artificial Sequence
  Description of Artificial Sequence Synthetic polynucleotide
gactcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggggtaccggcggcgatggcggcgatggccgagcggcccttccagtgcaggatctgtatgcgcaacttttctcggtcctccaacctgacccggcacatcagaacccatacaggcgaaaagcctttcgcctgcgacatttgtgggagaaaatttgctcggtccgacgccctgtccgagcataccaagatccacaccggctctcagaaaccattccagtgccgcatttgtatgcggaatttttccgactcctccgccctgaccacccatatccgcactcacaccggagagaagccctttgcttgcgacatttgtggcaggaaatttgctgactcctccgacctgtccgagcacactaagatccatactgggtcacagaaacctttccagtgccggatttgtatgagaaactttagccagtccggcaacctgtcccagcacatcagaacccatacaggcgaaaagcctttcgcctgcgacatttgtgggagaaaatttgctgaccggtccgacctgacccggcataccaagatccacaccggctctcagaaaccattccagtgccgcatttgtatgcggaatttttcccggtccgacaacctgacccggcacatcagaacacatactgggctgagaggatccaattctggtgatcctcggagacacagtctgggcggttctcgtaaacccgatctgattgcctataaaaactttgatctgctggtcattgttcttaagccttgagcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttctt

SEQ ID NO: 160 - DNA, 726 bp, Artificial Sequence
  Description of Artificial Sequence Synthetic polynucleotide
atggcggcgatggccgagcggcccttccagtgcaggatctgtatgcgcaacttttctcggtcctccaacctgacccggcacatcagaacccatacaggcgaaaagcctttcgcctgcgacatttgtgggagaaaatttgctcggtccgacgccctgtccgagcataccaagatccacaccggctctcagaaaccattccagtgccgcatttgtatgcggaatttttccgactcctccgccctgaccacccatatccgcactcacaccggagagaagccctttgcttgcgacatttgtggcaggaaatttgctgactcctccgacctgtccgagcacactaagatccatactgggtcacagaaacctttccagtgccggatttgtatgagaaactttagccagtccggcaacctgtcccagcacatcagaacccatacaggcgaaaagcctttcgcctgcgacatttgtgggagaaaatttgctgaccggtccgacctgacccggcataccaagatccacaccggctctcagaaaccattccagtgccgcatttgtatgcggaatttttcccggtccgacaacctgacccggcacatcagaacacatactgggctgagaggatccaattctggtgatcctcggagacacagtctgggcggttctcgtaaacccgatctgattgcctataaaaactttgatctgctggtcattgttcttaagccttga

SEQ ID NO: 161 - protein, 241 aa, Artificial Sequence
  Description of Artificial Sequence Synthetic polypeptide
MAAMAERPFQCRICMRNFSRSSNLTRHIRTHTGEKPFACDICGRKFARSDALSEHTKIHTGSQKPFQCRICMRNFSDSSALTTHIRTHTGEKPFACDICGRKFADSSDLSEHTKIHTGSQKPFQCRICMRNFSQSGNLSQHIRTHTGEKPFACDICGRKFADRSDLTRHTKIHTGSQKPFQCRICMRNFSRSDNLTRHIRTHTGLRGSNSGDPRRHSLGGSRKPDLIAYKNFDLLVIVLKP

SEQ ID NO: 162 - DNA, 21 bp, Homo sapiens
gaggccaaacccttcctggag

SEQ ID NO: 163 - DNA, 141 bp, Artificial Sequence
  Description of Artificial Sequence Synthetic polynucleotide
ctcttagacataacacaccagggtcaatacaactttgaagctagtctagtgcaagctaacagttgcttgaattcacaggctccaggaagggtttggcctctgattagggtgggggcgtgggtggggtagaagaggactggc

SEQ ID NO: 164 - DNA, 20 bp, Artificial Sequence
  Description of Artificial Sequence Synthetic primer
actcttagacataacacacc

SEQ ID NO: 165 - DNA, 19 bp, Artificial Sequence
  Description of Artificial Sequence Synthetic primer
aagagagccttccgaaaga

SEQ ID NO: 166 - DNA, 4405 bp, Artificial Sequence
  Description of Artificial Sequence Synthetic polynucleotide
gactcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttatggactacaaagaccatgacggtgattataaagatcatgacatcgattacaaggatgacgatgacaagatggcccccaagaagaagaggaaggtgggcattcacggggtaccgatggcggcgatggccgagcggcccttccagtgcaggatctgtatgcgcaacttttctcggtcctccaacctgacccggcacatcagaacccatacaggcgaaaagcctttcgcctgcgacatttgtgggagaaaatttgctcggtccgacgccctgtccgagcataccaagatccacaccggctctcagaaaccattccagtgccgcatttgtatgcggaatttttccgactcctccgccctgaccacccatatccgcactcacaccggagagaagccctttgcttgcgacatttgtggcaggaaatttgctgactcctccgacctgtccgagcacactaagatccatactgggtcacagaaacctttccagtgccggatttgtatgagaaactttagccagtccggcaacctgtcccagcacatcagaacccatacaggcgaaaagcctttcgcctgcgacatttgtgggagaaaatttgctgaccggtccgacctgacccggcataccaagatccacaccggctctcagaaaccattccagtgccgcatttgtatgcggaatttttcccggtccgacaacctgacccggcacatcagaacacatactgggctgagaggatccggcggcggcggcggctccggcggcggcggcggctccggcggcggcggcggctccggcggcggcggcggctccggcggcggcggcggctccggcggcggcggcggctccatggcggcgatggccgagcggcccttccagtgcaggatctgtatgcgcaacttttctcggtccgaccacctgacccggcacatcagaacccatacaggcgaaaagcctttcgcctgcgacatttgtgggagaaaatttgctacctccggccacctgacccggcataccaagatccacaccggctctcagaaaccattccagtgccgcatttgtatgcggaatttttcccggtccgacgccctgacccggcatatccgcactcacaccggagagaagccctttgcttgcgacatttgtggcaggaaatttgctgaccggtcccacctgacccggcacactaagatccatactgggtcacagaaacctttccagtgccggatttgtatgagaaactttagccggtccgaccacctgacccggcacatcagaacccatacaggcgaaaagcctttcgcctgcgacatttgtgggagaaaatttgctacctccggccacctgacccggcataccaagatccacaccggctctcagaaaccattccagtgccgcatttgtatgcggaatttttcccggtccgacaacctgaccacccacatcagaacacatactgggctgagatgagcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttctt

SEQ ID NO: 167 - DNA, 1467 bp, Artificial Sequence
  Description of Artificial Sequence Synthetic polynucleotide
atggactacaaagaccatgacggtgattataaagatcatgacatcgattacaaggatgacgatgacaagatggcccccaagaagaagaggaaggtgggcattcacggggtaccgatggcggcgatggccgagcggcccttccagtgcaggatctgtatgcgcaacttttctcggtcctccaacctgacccggcacatcagaacccatacaggcgaaaagcctttcgcctgcgacatttgtgggagaaaatttgctcggtccgacgccctgtccgagcataccaagatccacaccggctctcagaaaccattccagtgccgcatttgtatgcggaatttttccgactcctccgccctgaccacccatatccgcactcacaccggagagaagccctttgcttgcgacatttgtggcaggaaatttgctgactcctccgacctgtccgagcacactaagatccatactgggtcacagaaacctttccagtgccggatttgtatgagaaactttagccagtccggcaacctgtcccagcacatcagaacccatacaggcgaaaagcctttcgcctgcgacatttgtgggagaaaatttgctgaccggtccgacctgacccggcataccaagatccacaccggctctcagaaaccattccagtgccgcatttgtatgcggaatttttcccggtccgacaacctgacccggcacatcagaacacatactgggctgagaggatccggcggcggcggcggctccggcggcggcggcggctccggcggcggcggcggctccggcggcggcggcggctccggcggcggcggcggctccggcggcggcggcggctccatggcggcgatggccgagcggcccttccagtgcaggatctgtatgcgcaacttttctcggtccgaccacctgacccggcacatcagaacccatacaggcgaaaagcctttcgcctgcgacatttgtgggagaaaatttgctacctccggccacctgacccggcataccaagatccacaccggctctcagaaaccattccagtgccgcatttgtatgcggaatttttcccggtccgacgccctgacccggcatatccgcactcacaccggagagaagccctttgcttgcgacatttgtggcaggaaatttgctgaccggtcccacctgacccggcacactaagatccatactgggtcacagaaacctttccagtgccggatttgtatgagaaactttagccggtccgaccacctgacccggcacatcagaacccatacaggcgaaaagcctttcgcctgcgacatttgtgggagaaaatttgctacctccggccacctgacccggcataccaagatccacaccggctctcagaaaccattccagtgccgcatttgtatgcggaatttttcccggtccgacaacctgaccacccacatcagaacacatactgggctgagatga

SEQ ID NO: 168 - protein, 488 aa, Artificial Sequence
  Description of Artificial Sequence Synthetic polypeptide
MDYKDHDGDYKDHDIDYKDDDDKMAPKKKRKVGIHGVPMAAMAERPFQCRICMRNFSRSSNLTRHIRTHTGEKPFACDICGRKFARSDALSEHTKIHTGSQKPFQCRICMRNFSDSSALTTHIRTHTGEKPFACDICGRKFADSSDLSEHTKIHTGSQKPFQCRICMRNFSQSGNLSQHIRTHTGEKPFACDICGRKFADRSDLTRHTKIHTGSQKPFQCRICMRNFSRSDNLTRHIRTHTGLRGSGGGGGSGGGGGSGGGGGSGGGGGSGGGGGSGGGGGSMAAMAERPFQCRICMRNFSRSDHLTRHIRTHTGEKPFACDICGRKFATSGHLTRHTKIHTGSQKPFQCRICMRNFSRSDALTRHIRTHTGEKPFACDICGRKFADRSHLTRHTKIHTGSQKPFQCRICMRNFSRSDHLTRHIRTHTGEKPFACDICGRKFATSGHLTRHTKIHTGSQKPFQCRICMRNFSRSDNLTTHIRTHTGLR

SEQ ID NO: 169 - DNA, 21 bp, Artificial Sequence
  Description of Artificial Sequence Synthetic oligonucleotide
tagggtgggggcgtgggtggg

SEQ ID NO: 170 - DNA, 20 bp, Artificial Sequence
  Description of Artificial Sequence Synthetic primer
tgattccagtgcaaagtcca

SEQ ID NO: 171 - DNA, 20 bp, Artificial Sequence
  Description of Artificial Sequence Synthetic primer
agagagccttccgaaagagg

SEQ ID NO: 172 - DNA, 3940 bp, Artificial Sequence
  Description of Artificial Sequence Synthetic polynucleotide
gactcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggggtaccggcggcgatggcggcgatggccgagcggcccttcgcctgcgacatttgtgggagaaaatttgctgatcagtccggcaacctgacccggcataccaagatccacaccggctctcagaaaccattccagtgcaggatctgtatgcgcaacttttctcggtccgacaacctgtcccagcacatcagaacccatacaggcgaaaagccttttgcttgcgacatttgtggcaggaaatttgctacctccggcgacctgtcccagcacactaagatccatactgggtcacagaaacctttccagtgccgcatttgtatgcggaatttttccacctccggctccctgacccggcatatccgcactcacaccggagagaagccctttgcatgcgacatttgtggacggaaatttgctcggtccgacgccctgacccggcataccaagattcacactgggtctcagaaacctttccagtgcaggatttgtatgagaaatttttccacctccggcgacctgtccgagcacatcagaacccatacaggcgaaaagccttttgcttgcgacatttgtggcaggaaatttgctcagtccggcaacctgtccgagcacactaagatccatactgggtcacagaaacctttccagtgccgcatttgtatgcggaatttttcccagtccggcgacctgtcccagcacatcagaacccatacaggcgaaaagccttttgcttgcgacatttgtggcaggaaatttgctcggtcctccgccctgacccggcacactaagatccatactgggtcacagaaacctttccagtgccgcatttgtatgcggaatttttcccggtccgacgccctgtccgagcacatcagaacacatactgggctgagaggatccaattctggtgatcctcggagacacagtctgggcggttctcgtaaacccgatctgattgcctataaaaactttgatctgctggtcattgttcttaagccttgagcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttctt

SEQ ID NO: 173 - DNA, 987 bp, Artificial Sequence
  Description of Artificial Sequence Synthetic polynucleotide
atggcggcgatggccgagcggcccttcgcctgcgacatttgtgggagaaaatttgctgatcagtccggcaacctgacccggcataccaagatccacaccggctctcagaaaccattccagtgcaggatctgtatgcgcaacttttctcggtccgacaacctgtcccagcacatcagaacccatacaggcgaaaagccttttgcttgcgacatttgtggcaggaaatttgctacctccggcgacctgtcccagcacactaagatccatactgggtcacagaaacctttccagtgccgcatttgtatgcggaatttttccacctccggctccctgacccggcatatccgcactcacaccggagagaagccctttgcatgcgacatttgtggacggaaatttgctcggtccgacgccctgacccggcataccaagattcacactgggtctcagaaacctttccagtgcaggatttgtatgagaaatttttccacctccggcgacctgtccgagcacatcagaacccatacaggcgaaaagccttttgcttgcgacatttgtggcaggaaatttgctcagtccggcaacctgtccgagcacactaagatccatactgggtcacagaaacctttccagtgccgcatttgtatgcggaatttttcccagtccggcgacctgtcccagcacatcagaacccatacaggcgaaaagccttttgcttgcgacatttgtggcaggaaatttgctcggtcctccgccctgacccggcacactaagatccatactgggtcacagaaacctttccagtgccgcatttgtatgcggaatttttcccggtccgacgccctgtccgagcacatcagaacacatactgggctgagaggatccaattctggtgatcctcggagacacagtctgggcggttctcgtaaacccgatctgattgcctataaaaactttgatctgctggtcattgttcttaagccttga

SEQ ID NO: 174 - protein, 328 aa, Artificial Sequence
  Description of Artificial Sequence Synthetic polypeptide
MAAMAERPFACDICGRKFADQSGNLTRHTKIHTGSQKPFQCRICMRNFSRSDNLSQHIRTHTGEKPFACDICGRKFATSGDLSQHTKIHTGSQKPFQCRICMRNFSTSGSLTRHIRTHTGEKPFACDICGRKFARSDALTRHTKIHTGSQKPFQCRICMRNFSTSGDLSEHIRTHTGEKPFACDICGRKFAQSGNLSEHTKIHTGSQKPFQCRICMRNFSQSGDLSQHIRTHTGEKPFACDICGRKFARSSALTRHTKIHTGSQKPFQCRICMRNFSRSDALSEHIRTHTGLRGSNSGDPRRHSLGGSRKPDLIAYKNFDLLVIVLKP

SEQ ID NO: 175 - DNA, 30 bp, Homo sapiens
ctggtgacacaacctgtggttactaaggaa

SEQ ID NO: 176 - DNA, 138 bp, Artificial Sequence
  Description of Artificial Sequence Synthetic polynucleotide
taatttttctttttcttcttttttcctttttgcaaaaacccaaaatattttagctcctactcagactgttagactctggtgacacaacctgtggttactaaggaaactgccatctccaaactagaaatgccatcttcc

SEQ ID NO: 177 - DNA, 22 bp, Artificial Sequence
  Description of Artificial Sequence Synthetic primer
ttggctctttagcttgtgtttc

SEQ ID NO: 178 - DNA, 22 bp, Artificial Sequence
  Description of Artificial Sequence Synthetic primer
ggcatttctagtttggagatgg

SEQ ID NO: 179 - DNA, 4411 bp, Artificial Sequence
  Description of Artificial Sequence Synthetic polynucleotide
gactcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttatggactacaaagaccatgacggtgattataaagatcatgacatcgattacaaggatgacgatgacaagatggcccccaagaagaagaggaaggtgggcattcacggggtaccggcggcgatggcggcgatggccgagcggcccttccagtgcaggatctgtatgcgcaacttttctcagtccggcgacctgacccggcacatcagaacccatacaggcgaaaagcctttcgcctgcgacatttgtgggagaaaatttgctcggtccgacaacctgtccgagcataccaagatccacaccggctctcagaaaccattccagtgccgcatttgtatgcggaatttttccgaccggtccgccctgtccgagcatatccgcactcacaccggagagaagccctttgcttgcgacatttgtggcaggaaatttgctcggtcctccgccctgtccgagcacactaagatccatactgggtcacagaaacctttccagtgccggatttgtatgagaaactttagccggtcctcccacctgacccggcacatcagaacccatacaggcgaaaagcctttcgcctgcgacatttgtgggagaaaatttgctcggtccgacgccctgacccggcataccaagatccacaccggctctcagaaaccattccagtgccgcatttgtatgcggaatttttcccggtccgacgccctgtccgagcacatcagaacacatactgggctgagaggatccggcggcggcggcggctccggcggcggcggcggctccggcggcggcggcggctccggcggcggcggcggctccggcggcggcggcggctccggcggcggcggcggctccatggcggcgatggccgagcggcccttccagtgcaggatctgtatgcgcaacttttctcagtccggccacctgacccggcacatcagaacccatacaggcgaaaagcctttcgcctgcgacatttgtgggagaaaatttgctcggtccgacgccctgacccggcataccaagatccacaccggctctcagaaaccattccagtgccgcatttgtatgcggaatttttccacctccggcgacctgtccgagcatatccgcactcacaccggagagaagccctttgcttgcgacatttgtggcaggaaatttgctcggtcctccgacctgacccggcacactaagatccatactgggtcacagaaacctttccagtgccggatttgtatgagaaactttagccggtccgaccacctgtcccagcacatcagaacccatacaggcgaaaagcctttcgcctgcgacatttgtgggagaaaatttgctgaccggtccgacctgacccggcataccaagatccacaccggctctcagaaaccattccagtgccgcatttgtatgcggaatttttcccggtccgacgccctgtccgagcacatcagaacacatactgggctgagatgagcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttctt

SEQ ID NO: 180 - DNA, 1473 bp, Artificial Sequence
  Description of Artificial Sequence Synthetic polynucleotide
atggactacaaagaccatgacggtgattataaagatcatgacatcgattacaaggatgacgatgacaagatggcccccaagaagaagaggaaggtgggcattcacggggtaccggcggcgatggcggcgatggccgagcggcccttccagtgcaggatctgtatgcgcaacttttctcagtccggcgacctgacccggcacatcagaacccatacaggcgaaaagcctttcgcctgcgacatttgtgggagaaaatttgctcggtccgacaacctgtccgagcataccaagatccacaccggctctcagaaaccattccagtgccgcatttgtatgcggaatttttccgaccggtccgccctgtccgagcatatccgcactcacaccggagagaagccctttgcttgcgacatttgtggcaggaaatttgctcggtcctccgccctgtccgagcacactaagatccatactgggtcacagaaacctttccagtgccggatttgtatgagaaactttagccggtcctcccacctgacccggcacatcagaacccatacaggcgaaaagcctttcgcctgcgacatttgtgggagaaaatttgctcggtccgacgccctgacccggcataccaagatccacaccggctctcagaaaccattccagtgccgcatttgtatgcggaatttttcccggtccgacgccctgtccgagcacatcagaacacatactgggctgagaggatccggcggcggcggcggctccggcggcggcggcggctccggcggcggcggcggctccggcggcggcggcggctccggcggcggcggcggctccggcggcggcggcggctccatggcggcgatggccgagcggcccttccagtgcaggatctgtatgcgcaacttttctcagtccggccacctgacccggcacatcagaacccatacaggcgaaaagcctttcgcctgcgacatttgtgggagaaaatttgctcggtccgacgccctgacccggcataccaagatccacaccggctctcagaaaccattccagtgccgcatttgtatgcggaatttttccacctccggcgacctgtccgagcatatccgcactcacaccggagagaagccctttgcttgcgacatttgtggcaggaaatttgctcggtcctccgacctgacccggcacactaagatccatactgggtcacagaaacctttccagtgccggatttgtatgagaaactttagccggtccgaccacctgtcccagcacatcagaacccatacaggcgaaaagcctttcgcctgcgacatttgtgggagaaaatttgctgaccggtccgacctgacccggcataccaagatccacaccggctctcagaaaccattccagtgccgcatttgtatgcggaatttttcccggtccgacgccctgtccgagcacatcagaacacatactgggctgagatga

SEQ ID NO: 181 - protein, 490 aa, Artificial Sequence
  Description of Artificial Sequence Synthetic polypeptide
MDYKDHDGDYKDHDIDYKDDDDKMAPKKKRKVGIHGVPAAMAAMAERPFQCRICMRNFSQSGDLTRHIRTHTGEKPFACDICGRKFARSDNLSEHTKIHTGSQKPFQCRICMRNFSDRSALSEHIRTHTGEKPFACDICGRKFARSSALSEHTKIHTGSQKPFQCRICMRNFSRSSHLTRHIRTHTGEKPFACDICGRKFARSDALTRHTKIHTGSQKPFQCRICMRNFSRSDALSEHIRTHTGLRGSGGGGGSGGGGGSGGGGGSGGGGGSGGGGGSGGGGGSMAAMAERPFQCRICMRNFSQSGHLTRHIRTHTGEKPFACDICGRKFARSDALTRHTKIHTGSQKPFQCRICMRNFSTSGDLSEHIRTHTGEKPFACDICGRKFARSSDLTRHTKIHTGSQKPFQCRICMRNFSRSDHLSQHIRTHTGEKPFACDICGRKFADRSDLTRHTKIHTGSQKPFQCRICMRNFSRSDALSEHIRTHTGLR

SEQ ID NO: 182 - DNA, 3679 bp, Artificial Sequence
  Description of Artificial Sequence Synthetic polynucleotide
gactcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggggtaccggcggcgatggcggcgatggccgagcggcccttccagtgcaggatctgtatgcgcaacttttctcagtccggcgacctgacccggcacatcagaacccatacaggcgaaaagcctttcgcctgcgacatttgtgggagaaaatttgctcggtccgacaacctgtccgagcataccaagatccacaccggctctcagaaaccattccagtgccgcatttgtatgcggaatttttccgaccggtccgccctgtccgagcatatccgcactcacaccggagagaagccctttgcttgcgacatttgtggcaggaaatttgctcggtcctccgccctgtccgagcacactaagatccatactgggtcacagaaacctttccagtgccggatttgtatgagaaactttagccggtcctcccacctgacccggcacatcagaacccatacaggcgaaaagcctttcgcctgcgacatttgtgggagaaaatttgctcggtccgacgccctgacccggcataccaagatccacaccggctctcagaaaccattccagtgccgcatttgtatgcggaatttttcccggtccgacgccctgtccgagcacatcagaacacatactgggctgagaggatccaattctggtgatcctcggagacacagtctgggcggttctcgtaaacccgatctgattgcctataaaaactttgatctgctggtcattgttcttaagccttgagcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttctt

SEQ ID NO: 183 - DNA, 726 bp, Artificial Sequence
  Description of Artificial Sequence Synthetic polynucleotide
atggcggcgatggccgagcggcccttccagtgcaggatctgtatgcgcaacttttctcagtccggcgacctgacccggcacatcagaacccatacaggcgaaaagcctttcgcctgcgacatttgtgggagaaaatttgctcggtccgacaacctgtccgagcataccaagatccacaccggctctcagaaaccattccagtgccgcatttgtatgcggaatttttccgaccggtccgccctgtccgagcatatccgcactcacaccggagagaagccctttgcttgcgacatttgtggcaggaaatttgctcggtcctccgccctgtccgagcacactaagatccatactgggtcacagaaacctttccagtgccggatttgtatgagaaactttagccggtcctcccacctgacccggcacatcagaacccatacaggcgaaaagcctttcgcctgcgacatttgtgggagaaaatttgctcggtccgacgccctgacccggcataccaagatccacaccggctctcagaaaccattccagtgccgcatttgtatgcggaatttttcccggtccgacgccctgtccgagcacatcagaacacatactgggctgagaggatccaattctggtgatcctcggagacacagtctgggcggttctcgtaaacccgatctgattgcctataaaaactttgatctgctggtcattgttcttaagccttga

SEQ ID NO: 184 - protein, 241 aa, Artificial Sequence
  Description of Artificial Sequence Synthetic polypeptide
MAAMAERPFQCRICMRNFSQSGDLTRHIRTHTGEKPFACDICGRKFARSDNLSEHTKIHTGSQKPFQCRICMRNFSDRSALSEHIRTHTGEKPFACDICGRKFARSSALSEHTKIHTGSQKPFQCRICMRNFSRSSHLTRHIRTHTGEKPFACDICGRKFARSDALTRHTKIHTGSQKPFQCRICMRNFSRSDALSEHIRTHTGLRGSNSGDPRRHSLGGSRKPDLIAYKNFDLLVIVLKP

SEQ ID NO: 185 - DNA, 3664 bp, Artificial Sequence
  Description of Artificial Sequence Synthetic polynucleotide
gactcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttatggcggcgatggccgagcggcccttccagtgcaggatctgtatgcgcaacttttctcagtccggccacctgacccggcacatcagaacccatacaggcgaaaagcctttcgcctgcgacatttgtgggagaaaatttgctcggtccgacgccctgacccggcataccaagatccacaccggctctcagaaaccattccagtgccgcatttgtatgcggaatttttccacctccggcgacctgtccgagcatatccgcactcacaccggagagaagccctttgcttgcgacatttgtggcaggaaatttgctcggtcctccgacctgacccggcacactaagatccatactgggtcacagaaacctttccagtgccggatttgtatgagaaactttagccggtccgaccacctgtcccagcacatcagaacccatacaggcgaaaagcctttcgcctgcgacatttgtgggagaaaatttgctgaccggtccgacctgacccggcataccaagatccacaccggctctcagaaaccattccagtgccgcatttgtatgcggaatttttcccggtccgacgccctgtccgagcacatcagaacacatactgggctgagaggatccaattctggtgatcctcggagacacagtctgggcggttctcgtaaacccgatctgattgcctataaaaactttgatctgctggtcattgttcttaagccttgagcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttctt

SEQ ID NO: 186 - DNA, 726 bp, Artificial Sequence
  Description of Artificial Sequence Synthetic polynucleotide
atggcggcgatggccgagcggcccttccagtgcaggatctgtatgcgcaacttttctcagtccggccacctgacccggcacatcagaacccatacaggcgaaaagcctttcgcctgcgacatttgtgggagaaaatttgctcggtccgacgccctgacccggcataccaagatccacaccggctctcagaaaccattccagtgccgcatttgtatgcggaatttttccacctccggcgacctgtccgagcatatccgcactcacaccggagagaagccctttgcttgcgacatttgtggcaggaaatttgctcggtcctccgacctgacccggcacactaagatccatactgggtcacagaaacctttccagtgccggatttgtatgagaaactttagccggtccgaccacctgtcccagcacatcagaacccatacaggcgaaaagcctttcgcctgcgacatttgtgggagaaaatttgctgaccggtccgacctgacccggcataccaagatccacaccggctctcagaaaccattccagtgccgcatttgtatgcggaatttttcccggtccgacgccctgtccgagcacatcagaacacatactgggctgagaggatccaattctggtgatcctcggagacacagtctgggcggttctcgtaaacccgatctgattgcctataaaaactttgatctgctggtcattgttcttaagccttga

SEQ ID NO: 187 - protein, 241 aa, Artificial Sequence
  Description of Artificial Sequence Synthetic polypeptide
MAAMAERPFQCRICMRNFSQSGHLTRHIRTHTGEKPFACDICGRKFARSDALTRHTKIHTGSQKPFQCRICMRNFSTSGDLSEHIRTHTGEKPFACDICGRKFARSSDLTRHTKIHTGSQKPFQCRICMRNFSRSDHLSQHIRTHTGEKPFACDICGRKFADRSDLTRHTKIHTGSQKPFQCRICMRNFSRSDALSEHIRTHTGLRGSNSGDPRRHSLGGSRKPDLIAYKNFDLLVIVLKP

SEQ ID NO: 188 - DNA, 21 bp, Homo sapiens
ctggtggggctgctccaggca

SEQ ID NO: 189 - DNA, 21 bp, Homo sapiens
ctggccagggcgcctgtggga

SEQ ID NO: 190 - DNA, 149 bp, Artificial Sequence
  Description of Artificial Sequence Synthetic polynucleotide
tttcccttccgctcacctccgcctgagcagtggagaaggcggcactctggtggggctgctccaggcatgaattcatgatcccacaggcgccctggccagtcgtctgggcggtgctacaactgggctggcggccaggatggttcttaggt

SEQ ID NO: 191 - DNA, 19 bp, Artificial Sequence
  Description of Artificial Sequence Synthetic primer
gcctgagcagtggagaagg

SEQ ID NO: 192 - DNA, 19 bp, Artificial Sequence
  Description of Artificial Sequence Synthetic primer
ggactgagggtggaaggtc

SEQ ID NO: 193 - DNA, 197 bp, Homo sapiens
actcttagacataacacaccagggtcaatacaactttgaagctagtctagtgcaagctaacagttgcttttatcacaggctccaggaagggtttggcctctgattagggtgggggcgtgggtggggtagaagaggactggcagacctctccatcggtggccgtttgcccaggggggcctctttcggaaggctctctt

SEQ ID NO: 194 - DNA, 3754 bp, Artificial Sequence
  Description of Artificial Sequence Synthetic polynucleotide
gactcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttatggcggcgatggccgagcggcccttcgcctgcgacatttgtgggagaaaatttgctgatcagtccggcaacctgacccggcataccaagatccacaccggctctcagaaaccattccagtgcaggatctgtatgcgcaacttttctcggtccgacaacctgtccgagcacatcagaacccatacaggcgaaaagccttttgcttgcgacatttgtggcaggaaatttgctgactcctccgccctgtcccagcacactaagatccatactgggtcacagaaacctttccagtgccgcatttgtatgcggaatttttcccagtccggctccctgtcccagcatatccgcactcacaccggagagaagccctttgcatgcgacatttgtggacggaaatttgctgaccggtcccacctgacccggcataccaagattcacactgggtctcagaaacctttccagtgcaggatttgtatgagaaatttttcccagtccggcgacctgtccgagcacatcagaacccatacaggcgaaaagccttttgcttgcgacatttgtggcaggaaatttgctcggtcctccgccctgacccggcacactaagatccatactgggtcacagaaacctttccagtgccgcatttgtatgcggaatttttcccggtccgactccctgtcccagcacatcagaacacatactgggctgagaggatccaattctggtgatcctcggagacacagtctgggcggttctcgtaaacccgatctgattgcctataaaaactttgatctgctggtcattgttcttaagccttgagcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttctt

SEQ ID NO: 195 - DNA, 816 bp, Artificial Sequence
  Description of Artificial Sequence Synthetic polynucleotide
atggcggcgatggccgagcggcccttcgcctgcgacatttgtgggagaaaatttgctgatcagtccggcaacctgacccggcataccaagatccacaccggctctcagaaaccattccagtgcaggatctgtatgcgcaacttttctcggtccgacaacctgtccgagcacatcagaacccatacaggcgaaaagccttttgcttgcgacatttgtggcaggaaatttgctgactcctccgccctgtcccagcacactaagatccatactgggtcacagaaacctttccagtgccgcatttgtatgcggaatttttcccagtccggctccctgtcccagcatatccgcactcacaccggagagaagccctttgcatgcgacatttgtggacggaaatttgctgaccggtcccacctgacccggcataccaagattcacactgggtctcagaaacctttccagtgcaggatttgtatgagaaatttttcccagtccggcgacctgtccgagcacatcagaacccatacaggcgaaaagccttttgcttgcgacatttgtggcaggaaatttgctcggtcctccgccctgacccggcacactaagatccatactgggtcacagaaacctttccagtgccgcatttgtatgcggaatttttcccggtccgactccctgtcccagcacatcagaacacatactgggctgagaggatccaattctggtgatcctcggagacacagtctgggcggttctcgtaaacccgatctgattgcctataaaaactttgatctgctggtcattgttcttaagccttga

SEQ ID NO: 196 - protein, 271 aa, Artificial Sequence
  Description of Artificial Sequence Synthetic polypeptide
MAAMAERPFACDICGRKFADQSGNLTRHTKIHTGSQKPFQCRICMRNFSRSDNLSEHIRTHTGEKPFACDICGRKFADSSALSQHTKIHTGSQKPFQCRICMRNFSQSGSLSQHIRTHTGEKPFACDICGRKFADRSHLTRHTKIHTGSQKPFQCRICMRNFSQSGDLSEHIRTHTGEKPFACDICGRKFARSSALTRHTKIHTGSQKPFQCRICMRNFSRSDSLSQHIRTHTGLRGSNSGDPRRHSLGGSRKPDLIAYKNFDLLVIVLKP

SEQ ID NO: 197 - DNA, 24 bp, Homo sapiens
atggtgccaggcataatccaggaa

SEQ ID NO: 198 - DNA, 130 bp, Artificial Sequence
  Description of Artificial Sequence Synthetic polynucleotide
gaatttcattctgttctcagttttcctggattatgcctggcaccattaaagaaaatatcatatgtggtgtttcctatgatgaatatagatacagaagcgtcatcaaagcatgccaactagaagaggtaag

SEQ ID NO: 199 - DNA, 20 bp, Artificial Sequence
  Description of Artificial Sequence Synthetic primer
tggagccttcagagggtaaa

SEQ ID NO: 200 - DNA, 20 bp, Artificial Sequence
  Description of Artificial Sequence Synthetic primer
agttggcatgctttgatgac

SEQ ID NO: 201 - DNA, 32 bp, Artificial Sequence
  Description of Artificial Sequence Synthetic probe
ccattaaagaaaatatcatctttggtgtttcc

SEQ ID NO: 202 - DNA, 26 bp, Artificial Sequence
  Description of Artificial Sequence Synthetic probe
aaatatcatatgtggtgtttcctatg

SEQ ID NO: 203 - RNA, 36 nt, Artificial Sequence
  Description of Artificial Sequence Synthetic oligonucleotide
ggcgcaggcccggcugggcgguuuuagagcuaugcu

SEQ ID NO: 204 - DNA, 20 bp, Homo sapiens
ggcgcaggcccggctgggcg

SEQ ID NO: 205 - DNA, 20 bp, Artificial Sequence
  Description of Artificial Sequence Synthetic oligonucleotide
cctggtgcagtaccgcggcg

SEQ ID NO: 206 - DNA, 8506 bp, Artificial Sequence
  Description of Artificial Sequence Synthetic polynucleotide
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgggtcttcgagaagacctgttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagaggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtgccaccatggactataaggaccacgacggagactacaaggatcatgatattgattacaaagacgatgacgataagatggccccaaagaagaagcggaaggtcggtatccacggagtcccagcagccgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgccatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaagtaagaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 207 - DNA, 99 bp, Artificial Sequence; identical to SEQ ID NO: 134
  Description of Artificial Sequence Synthetic oligonucleotide

SEQ ID NO: 208 - protein, 33 aa, Artificial Sequence; identical to SEQ ID NO: 19
  Description of Artificial Sequence Synthetic polypeptide

SEQ ID NO: 209 - DNA, 111 bp, Artificial Sequence; identical to SEQ ID NO: 143
  Description of Artificial Sequence Synthetic polynucleotide

SEQ ID NO: 210 - protein, 37 aa, Artificial Sequence; identical to SEQ ID NO: 115
  Description of Artificial Sequence Synthetic polypeptide

SEQ ID NO: 211 - DNA, 105 bp, Artificial Sequence
  Description of Artificial Sequence Synthetic polynucleotide
aactctggtgatcctcggagacacagtctgggcggttctcgtaaacccgatattattcttgttaatgataatatttctcttattcttattcttgttgctaaacct

SEQ ID NO: 212 - protein, 35 aa, Artificial Sequence; identical to SEQ ID NO: 121
  Description of Artificial Sequence Synthetic polypeptide

SEQ ID NO: 213 - DNA, 222 bp, Artificial Sequence
  Description of Artificial Sequence Synthetic polynucleotide
aattctggtgatcctcggagacacagtctgggcggttctcgtaaacccgatctgattgcctataaaaactttgatctgctggtcattgttcttaagcctaaatactcccagaattctggtgatcctcggagacacagtctgggcggttctcgtaaacccgatggtgctatttatactgttggttctcctattgattatggtgttattgttgttactaaacct

SEQ ID NO: 214 - protein, 74 aa, Artificial Sequence
  Description of Artificial Sequence Synthetic polypeptide
NSGDPRRHSLGGSRKPDLIAYKNFDLLVIVLKPKYSQNSGDPRRHSLGGSRKPDGAIYTVGSPIDYGVIVVTKP

SEQ ID NO: 215 - DNA, 339 bp, Artificial Sequence
  Description of Artificial Sequence Synthetic polynucleotide
aattctggtgatcctcggagacacagtctgggcggttctcgtaaacccgatctgattgcctataaaaactttgatctgctggtcattgttcttaagcctaaatactcccagaattctggtgatcctcggagacacagtctgggcggttctcgtaaacccgatggtgctatttatactgttggttctcctattgattatggtgttattgttgttactaaacctaagtactcccagaactctggtgatcctcggagacacagtctgggcggttctcgtaaacccgatattattcttgttaatgataatatttctcttattcttattcttgttgctaaacct

SEQ ID NO: 216 - protein, 113 aa, Artificial Sequence
  Description of Artificial Sequence Synthetic polypeptide
NSGDPRRHSLGGSRKPDLIAYKNFDLLVIVLKPKYSQNSGDPRRHSLGGSRKPDGAIYTVGSPIDYGVIVVTKPKYSQNSGDPRRHSLGGSRKPDIILVNDNISLILILVAKP

SEQ ID NO: 217 - DNA, 3664 bp, Artificial Sequence
  Description of Artificial Sequence Synthetic polynucleotide
gactcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttatggcggcgatggccgagcggcccttccagtgcaggatctgtatgcgcaacttttctcagtccggcgacctgacccggcacatcagaacccatacaggcgaaaagcctttcgcctgcgacatttgtgggagaaaatttgctcggtccgacaacctgaccacccataccaagatccacaccggctctcagaaaccattccagtgccgcatttgtatgcggaatttttcccggtcctccgacctgacccggcatatccgcactcacaccggagagaagccctttgcttgcgacatttgtggcaggaaatttgctcggtccgacgccctgacccggcacactaagatccatactgggtcacagaaacctttccagtgccggatttgtatgagaaactttagccggtccgacgccctgtccgagcacatcagaacccatacaggcgaaaagcctttcgcctgcgacatttgtgggagaaaatttgctcggtcctccaacctgacccggcataccaagatccacaccggctctcagaaaccattccagtgccgcatttgtatgcggaatttttcccggtccgacgccctgaccacccacatcagaacacatactgggctgagaggatccaattctggtgatcctcggagacacagtctgggcggttctcgtaaacccgatctgattgcctataaaaactttgatctgctggtcattgttcttaagccttgagcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttctt

SEQ ID NO: 218 - DNA, 726 bp, Artificial Sequence
  Description of Artificial Sequence Synthetic polynucleotide
atggcggcgatggccgagcggcccttccagtgcaggatctgtatgcgcaacttttctcagtccggcgacctgacccggcacatcagaacccatacaggcgaaaagcctttcgcctgcgacatttgtgggagaaaatttgctcggtccgacaacctgaccacccataccaagatccacaccggctctcagaaaccattccagtgccgcatttgtatgcggaatttttcccggtcctccgacctgacccggcatatccgcactcacaccggagagaagccctttgcttgcgacatttgtggcaggaaatttgctcggtccgacgccctgacccggcacactaagatccatactgggtcacagaaacctttccagtgccggatttgtatgagaaactttagccggtccgacgccctgtccgagcacatcagaacccatacaggcgaaaagcctttcgcctgcgacatttgtgggagaaaatttgctcggtcctccaacctgacccggcataccaagatccacaccggctctcagaaaccattccagtgccgcatttgtatgcggaatttttcccggtccgacgccctgaccacccacatcagaacacatactgggctgagaggatccaattctggtgatcctcggagacacagtctgggcggttctcgtaaacccgatctgattgcctataaaaactttgatctgctggtcattgttcttaagccttga

SEQ ID NO: 219 - protein, 241 aa, Artificial Sequence
  Description of Artificial Sequence Synthetic polypeptide
MAAMAERPFQCRICMRNFSQSGDLTRHIRTHTGEKPFACDICGRKFARSDNLTTHTKIHTGSQKPFQCRICMRNFSRSSDLTRHIRTHTGEKPFACDICGRKFARSDALTRHTKIHTGSQKPFQCRICMRNFSRSDALSEHIRTHTGEKPFACDICGRKFARSSNLTRHTKIHTGSQKPFQCRICMRNFSRSDALTTHIRTHTGLRGSNSGDPRRHSLGGSRKPDLIAYKNFDLLVIVLKP

SEQ ID NO: 220 - DNA, 3787 bp, Artificial Sequence
  Description of Artificial Sequence Synthetic polynucleotide
gactcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttatggcggcgatggccgagcggcccttccagtgcaggatctgtatgcgcaacttttctcagtccggcgacctgacccggcacatcagaacccatacaggcgaaaagcctttcgcctgcgacatttgtgggagaaaatttgctcggtccgacaacctgaccacccataccaagatccacaccggctctcagaaaccattccagtgccgcatttgtatgcggaatttttcccggtcctccgacctgacccggcatatccgcactcacaccggagagaagccctttgcttgcgacatttgtggcaggaaatttgctcggtccgacgccctgacccggcacactaagatccatactgggtcacagaaacctttccagtgccggatttgtatgagaaactttagccggtccgacgccctgtccgagcacatcagaacccatacaggcgaaaagcctttcgcctgcgacatttgtgggagaaaatttgctcggtcctccaacctgacccggcataccaagatccacaccggctctcagaaaccattccagtgccgcatttgtatgcggaatttttcccggtccgacgccctgaccacccacatcagaacacatactgggctgagaggatccaattctggtgatcctcggagacacagtctgggcggttctcgtaaacccgatctgattgcctataaaaactttgatctgctggtcattgttcttaagcctaaatactcccagaattctggtgatcctcggagacacagtctgggcggttctcgtaaacccgatggtgctatttatactgttggttctcctattgattatggtgttattgttgttactaaaccttgagcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttctt

SEQ ID NO: 221 - DNA, 849 bp, Artificial Sequence
  Description of Artificial Sequence Synthetic polynucleotide
atggcggcgatggccgagcggcccttccagtgcaggatctgtatgcgcaacttttctcagtccggcgacctgacccggcacatcagaacccatacaggcgaaaagcctttcgcctgcgacatttgtgggagaaaatttgctcggtccgacaacctgaccacccataccaagatccacaccggctctcagaaaccattccagtgccgcatttgtatgcggaatttttcccggtcctccgacctgacccggcatatccgcactcacaccggagagaagccctttgcttgcgacatttgtggcaggaaatttgctcggtccgacgccctgacccggcacactaagatccatactgggtcacagaaacctttccagtgccggatttgtatgagaaactttagccggtccgacgccctgtccgagcacatcagaacccatacaggcgaaaagcctttcgcctgcgacatttgtgggagaaaatttgctcggtcctccaacctgacccggcataccaagatccacaccggctctcagaaaccattccagtgccgcatttgtatgcggaatttttcccggtccgacgccctgaccacccacatcagaacacatactgggctgagaggatccaattctggtgatcctcggagacacagtctgggcggttctcgtaaacccgatctgattgcctataaaaactttgatctgctggtcattgttcttaagcctaaatactcccagaattctggtgatcctcggagacacagtctgggcggttctcgtaaacccgatggtgctatttatactgttggttctcctattgattatggtgttattgttgttactaaaccttga

SEQ ID NO: 222 - protein, 282 aa, Artificial Sequence
  Description of Artificial Sequence Synthetic polypeptide
MAAMAERPFQCRICMRNFSQSGDLTRHIRTHTGEKPFACDICGRKFARSDNLTTHTKIHTGSQKPFQCRICMRNFSRSSDLTRHIRTHTGEKPFACDICGRKFARSDALTRHTKIHTGSQKPFQCRICMRNFSRSDALSEHIRTHTGEKPFACDICGRKFARSSNLTRHTKIHTGSQKPFQCRICMRNFSRSDALTTHIRTHTGLRGSNSGDPRRHSLGGSRKPDLIAYKNFDLLVIVLKPKYSQNSGDPRRHSLGGSRKPDGAIYTVGSPIDYGVIVVTKP

SEQ ID NO: 223 - DNA, 3904 bp, Artificial Sequence
  Description of Artificial Sequence Synthetic polynucleotide
gactcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttatggcggcgatggccgagcggcccttccagtgcaggatctgtatgcgcaacttttctcagtccggcgacctgacccggcacatcagaacccatacaggcgaaaagcctttcgcctgcgacatttgtgggagaaaatttgctcggtccgacaacctgaccacccataccaagatccacaccggctctcagaaaccattccagtgccgcatttgtatgcggaatttttcccggtcctccgacctgacccggcatatccgcactcacaccggagagaagccctttgcttgcgacatttgtggcaggaaatttgctcggtccgacgccctgacccggcacactaagatccatactgggtcacagaaacctttccagtgccggatttgtatgagaaactttagccggtccgacgccctgtccgagcacatcagaacccatacaggcgaaaagcctttcgcctgcgacatttgtgggagaaaatttgctcggtcctccaacctgacccggcataccaagatccacaccggctctcagaaaccattccagtgccgcatttgtatgcggaatttttcccggtccgacgccctgaccacccacatcagaacacatactgggctgagaggatccaattctggtgatcctcggagacacagtctgggcggttctcgtaaacccgatctgattgcctataaaaactttgatctgctggtcattgttcttaagcctaaatactcccagaattctggtgatcctcggagacacagtctgggcggttctcgtaaacccgatggtgctatttatactgttggttctcctattgattatggtgttattgttgttactaaacctaagtactcccagaactctggtgatcctcggagacacagtctgggcggttctcgtaaacccgatattattcttgttaatgataatatttctcttattcttattcttgttgctaaaccttgagcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttctt

SEQ ID NO: 224 - DNA, 966 bp, Artificial Sequence
  Description of Artificial Sequence Synthetic polynucleotide
atggcggcgatggccgagcggcccttccagtgcaggatctgtatgcgcaacttttctcagtccggcgacctgacccggcacatcagaacccatacaggcgaaaagcctttcgcctgcgacatttgtgggagaaaatttgctcggtccgacaacctgaccacccataccaagatccacaccggctctcagaaaccattccagtgccgcatttgtatgcggaatttttcccggtcctccgacctgacccggcatatccgcactcacaccggagagaagccctttgcttgcgacatttgtggcaggaaatttgctcggtccgacgccctgacccggcacactaagatccatactgggtcacagaaacctttccagtgccggatttgtatgagaaactttagccggtccgacgccctgtccgagcacatcagaacccatacaggcgaaaagcctttcgcctgcgacatttgtgggagaaaatttgctcggtcctccaacctgacccggcataccaagatccacaccggctctcagaaaccattccagtgccgcatttgtatgcggaatttttcccggtccgacgccctgaccacccacatcagaacacatactgggctgagaggatccaattctggtgatcctcggagacacagtctgggcggttctcgtaaacccgatctgattgcctataaaaactttgatctgctggtcattgttcttaagcctaaatactcccagaattctggtgatcctcggagacacagtctgggcggttctcgtaaacccgatggtgctatttatactgttggttctcctattgattatggtgttattgttgttactaaacctaagtactcccagaactctggtgatcctcggagacacagtctgggcggttctcgtaaacccgatattattcttgttaatgataatatttctcttattcttattcttgttgctaaaccttga

SEQ ID NO: 225 - protein, 321 aa, Artificial Sequence
  Description of Artificial Sequence Synthetic polypeptide
MAAMAERPFQCRICMRNFSQSGDLTRHIRTHTGEKPFACDICGRKFARSDNLTTHTKIHTGSQKPFQCRICMRNFSRSSDLTRHIRTHTGEKPFACDICGRKFARSDALTRHTKIHTGSQKPFQCRICMRNFSRSDALSEHIRTHTGEKPFACDICGRKFARSSNLTRHTKIHTGSQKPFQCRICMRNFSRSDALTTHIRTHTGLRGSNSGDPRRHSLGGSRKPDLIAYKNFDLLVIVLKPKYSQNSGDPRRHSLGGSRKPDGAIYTVGSPIDYGVIVVTKPKYSQNSGDPRRHSLGGSRKPDIILVNDNISLILILVAKP

SEQ ID NO: 226 - DNA, 21 bp, Unknown
  Description of Unknown KRAS target sequence
ttggagctggtggcgtaggca

SEQ ID NO: 227 - DNA, 137 bp, Artificial Sequence
  Description of Artificial Sequence Synthetic polynucleotide
aaaatgactgaatataaacttgtggtagttggagctggtggcgtaggcaagagttgagaatccgttgacgatacagctaattcagaatcattttgtggacgaatatgatccaacaatagaggtaaatcttgttttaa

SEQ ID NO: 228 - DNA, 31 bp, Artificial Sequence
  Description of Artificial Sequence Synthetic oligonucleotide
gactgaatataaacttgtggtagttggagct

SEQ ID NO: 229 - DNA, 21 bp, Artificial Sequence
  Description of Artificial Sequence Synthetic oligonucleotide
tcctcttgacctgctgtgtcg

SEQ ID NO: 230 - DNA, 618 bp, Artificial Sequence
  Description of Artificial Sequence Synthetic polynucleotide
atggcggcgatggccgagcggcccttccagtgcaggatctgtatgcgcaacttttctcggtcctccaacctgacccggcacatcagaacccatacaggcgaaaagcctttcgcctgcgacatttgtgggagaaaatttgctcggtccgacgccctgtccgagcataccaagatccacaccggctctcagaaaccattccagtgccgcatttgtatgcggaatttttccgactcctccgccctgaccacccatatccgcactcacaccggagagaagccctttgcttgcgacatttgtggcaggaaatttgctgactcctccgacctgtccgagcacactaagatccatactgggtcacagaaacctttccagtgccggatttgtatgagaaactttagccagtccggcaacctgtcccagcacatcagaacccatacaggcgaaaagcctttcgcctgcgacatttgtgggagaaaatttgctgaccggtccgacctgacccggcataccaagatccacaccggctctcagaaaccattccagtgccgcatttgtatgcggaatttttcccggtccgacaacctgacccggcacatcagaacacatactgggctgaga

SEQ ID NO: 231 - DNA, 879 bp, Artificial Sequence
  Description of Artificial Sequence Synthetic polynucleotide
atggcggcgatggccgagcggcccttcgcctgcgacatttgtgggagaaaatttgctgatcagtccggcaacctgacccggcataccaagatccacaccggctctcagaaaccattccagtgcaggatctgtatgcgcaacttttctcggtccgacaacctgtcccagcacatcagaacccatacaggcgaaaagccttttgcttgcgacatttgtggcaggaaatttgctacctccggcgacctgtcccagcacactaagatccatactgggtcacagaaacctttccagtgccgcatttgtatgcggaatttttccacctccggctccctgacccggcatatccgcactcacaccggagagaagccctttgcatgcgacatttgtggacggaaatttgctcggtccgacgccctgacccggcataccaagattcacactgggtctcagaaacctttccagtgcaggatttgtatgagaaatttttccacctccggcgacctgtccgagcacatcagaacccatacaggcgaaaagccttttgcttgcgacatttgtggcaggaaatttgctcagtccggcaacctgtccgagcacactaagatccatactgggtcacagaaacctttccagtgccgcatttgtatgcggaatttttcccagtccggcgacctgtcccagcacatcagaacccatacaggcgaaaagccttttgcttgcgacatttgtggcaggaaatttgctcggtcctccgccctgacccggcacactaagatccatactgggtcacagaaacctttccagtgccgcatttgtatgcggaatttttcccggtccgacgccctgtccgagcacatcagaacacatactgggctgaga

SEQ ID NO: 232 - DNA, 618 bp, Artificial Sequence
  Description of Artificial Sequence Synthetic polynucleotide
atggcggcgatggccgagcggcccttccagtgcaggatctgtatgcgcaacttttctcagtccggcgacctgacccggcacatcagaacccatacaggcgaaaagcctttcgcctgcgacatttgtgggagaaaatttgctcggtccgacaacctgtccgagcataccaagatccacaccggctctcagaaaccattccagtgccgcatttgtatgcggaatttttccgaccggtccgccctgtccgagcatatccgcactcacaccggagagaagccctttgcttgcgacatttgtggcaggaaatttgctcggtcctccgccctgtccgagcacactaagatccatactgggtcacagaaacctttccagtgccggatttgtatgagaaactttagccggtcctcccacctgacccggcacatcagaacccatacaggcgaaaagcctttcgcctgcgacatttgtgggagaaaatttgctcggtccgacgccctgacccggcataccaagatccacaccggctctcagaaaccattccagtgccgcatttgtatgcggaatttttcccggtccgacgccctgtccgagcacatcagaacacatactgggctgaga

SEQ ID NO: 233 - DNA, 621 bp, Artificial Sequence
  Description of Artificial Sequence Synthetic polynucleotide
atggcggcgatggccgagcggcccttccagtgcaggatctgtatgcgcaacttttctcagtccggccacctgacccggcacatcagaacccatacaggcgaaaagcctttcgcctgcgacatttgtgggagaaaatttgctcggtccgacgccctgacccggcataccaagatccacaccggctctcagaaaccattccagtgccgcatttgtatgcggaatttttccacctccggcgacctgtccgagcatatccgcactcacaccggagagaagccctttgcttgcgacatttgtggcaggaaatttgctcggtcctccgacctgacccggcacactaagatccatactgggtcacagaaacctttccagtgccggatttgtatgagaaactttagccggtccgaccacctgtcccagcacatcagaacccatacaggcgaaaagcctttcgcctgcgacatttgtgggagaaaatttgctgaccggtccgacctgacccggcataccaagatccacaccggctctcagaaaccattccagtgccgcatttgtatgcggaatttttcccggtccgacgccctgtccgagcacatcagaacacatactgggctgagatga

SEQ ID NO: 234 - DNA, 708 bp, Artificial Sequence
  Description of Artificial Sequence Synthetic polynucleotide
atggcggcgatggccgagcggcccttcgcctgcgacatttgtgggagaaaatttgctgatcagtccggcaacctgacccggcataccaagatccacaccggctctcagaaaccattccagtgcaggatctgtatgcgcaacttttctcggtccgacaacctgtccgagcacatcagaacccatacaggcgaaaagccttttgcttgcgacatttgtggcaggaaatttgctgactcctccgccctgtcccagcacactaagatccatactgggtcacagaaacctttccagtgccgcatttgtatgcggaatttttcccagtccggctccctgtcccagcatatccgcactcacaccggagagaagccctttgcatgcgacatttgtggacggaaatttgctgaccggtcccacctgacccggcataccaagattcacactgggtctcagaaacctttccagtgcaggatttgtatgagaaatttttcccagtccggcgacctgtccgagcacatcagaacccatacaggcgaaaagccttttgcttgcgacatttgtggcaggaaatttgctcggtcctccgccctgacccggcacactaagatccatactgggtcacagaaacctttccagtgccgcatttgtatgcggaatttttcccggtccgactccctgtcccagcacatcagaacacatactgggctgaga

SEQ ID NO: 235 - DNA, 618 bp, Artificial Sequence
  Description of Artificial Sequence Synthetic polynucleotide
atggcggcgatggccgagcggcccttccagtgcaggatctgtatgcgcaacttttctcagtccggcgacctgacccggcacatcagaacccatacaggcgaaaagcctttcgcctgcgacatttgtgggagaaaatttgctcggtccgacaacctgaccacccataccaagatccacaccggctctcagaaaccattccagtgccgcatttgtatgcggaatttttcccggtcctccgacctgacccggcatatccgcactcacaccggagagaagccctttgcttgcgacatttgtggcaggaaatttgctcggtccgacgccctgacccggcacactaagatccatactgggtcacagaaacctttccagtgccggatttgtatgagaaactttagccggtccgacgccctgtccgagcacatcagaacccatacaggcgaaaagcctttcgcctgcgacatttgtgggagaaaatttgctcggtcctccaacctgacccggcataccaagatccacaccggctctcagaaaccattccagtgccgcatttgtatgcggaatttttcccggtccgacgccctgaccacccacatcagaacacatactgggctgaga

SEQ ID NO: 236 - DNA, 1323 bp, Homo sapiens
ttaaaaaatagctaagaatagtgaaaacacccttgtaatttagagactctcagaaaaatgacagcaccatttagagcctggaattacagtttgacttcactgtgccttctctgccccaggctcccatggtggcaagggtttttggttgggggaaggggtattgaattgcctgtctttgagcaggaaaagaattacagttttccaggtaccttttgtgtgtatgtgctgattgagggcccattgagaatattttgacttttagggaagctccaaactctcaaaccacagggatcacaacacatacgtgtgtctgttatgacgttatatgtaagcatcacaacaggcagagaatgtctgcaccccaccctggaaaacagcctgactgtgccccatgggcaaaccagactagtttatagggggttctactctgaggtactgatggaccttgggtgctattcctgtgataaggaaggcagctagacaggacttgggagttatctgtagtgagatggctgaaaagcgatacagggctggctctatgccccaggtgtgcataagtaagagcagatagctgattccagtgcaaagtccatacaggtaataacataggccagaaaagagatatggcatctactcttagacataacacaccagggtcaatacaactttgaagctagtctagtgcaagctaacagttgcttttatcacaggctccaggaagggtttggcctctgattagggtgggggcgtgggtggggtagaagaggactggcagacctctccatcggtggccgtttgcccaggggggcctctttcggaaggctctcttggtgatggagaattggattttatttctcaatgggaatgaaataatttgtatgccatgccgtgtggactcccaaaattgtaaaggaggtgaagcttcccctgtctgcactctcccctcctcataattgtccatttttcatctgtcgggctgtccacccatccatcacatataggcacctatcaggtaccagctactgtgttaggatctgtgttcccaactgacttgcctccccctgacgtcatattcttttcctttttcctctcccttttcccttttcttctgacccaaactaggaattggggaaagggcctgataactttgtttctgctgaggtgtaactaataaataccaggaggcagcattttagttcacaagctcggagcacttactctgctctaggaactttacaaatacgcactcattttattttcatacaaaccctatgaagcatatactattattattcctattttacggatgagtccatattttaa

SEQ ID NO: 237 - DNA, 3159 bp, Homo sapiens
aagcttgagagacaagaaacattcttccattctactcatcttcttctctaatgaggagacaaccttaaaagcacagttacatagccataaaaattaatgattggctacctcagaatgaaaattcaatgtctcatttttttttaatattcttagaatcgttcactggttgtccagtgtgagtctcctgttgagatgtcttttgcagctttccttgaaacctttcattccaaactacatagtccaataattttgccaccaatcttctggttatattatgctcttgagtctgttgtctataaacttgattaggcattccttcccctcaccactcacctctgataacccagctgtgtgttggtatttagtatcaattcacaccagcaagttcagccctcttcaatcaatatagggccacacacggacttttgactgactactccccaagtatttcacattttggggccttatctccagtttctcaccacagttgttcatcactgtgtttcttactagccaggtgtttataaaaacactaatacctaacactattgatcacctactatagtgtcaggcgctgtaataatattattgtgatgatgatgattatgctgctctttctggcattgtcatacgtgtattgcttgtactactcactgaatctacacaactgcccttatgacatttaccctgttattattcctcttttaaggtaaatacatgaaaaatgcttcccactttgccttgcttactgcttattgctagtactgaacaaatgttagaactgaaacttagagaggttatgtggctttaccaaggtcccagagttcctagggtagagaacaggattgtctaccagacattttaattctagtactatgcatcttaaccattaccataggctgacttactctacagtgtccaacatattcactattaagatttatttaatgactttgaaacagtatttcatgtctaaatagaaaaactactaactcgcatttttaagaaaatattgtatcttggtttttcttcactgctggccagtttactaacaatctgaaataaaaagaaaaaaatatgataaactgctcccagtataaaatacagagctaagacaagaacgtttcattggctttgatttccctagggtccagcttcaaattaatttacttcctattcaagggaattcttaaatcagaaagaagatcttatcccatcttgttttgcctttgttttttcttgaataaaaaaaaaataagtaaaatttatttccctggcaaggtctgaaaacttttgttttctttaccacttccacaatgtatatgattgttactgagaaggcttatttaacttaagttacttgtccaggcatgagaatgagcaaaatcgttttttaaaaaattgttaaatgtatattaatgaaaaggttgaatcttttcattttctaccatgtattgctaaacaaagtatccacattgttagaaaaagatatataatgtcatgaataagagtttggctcaaattgttactcttcaattaaatttgacttattgttattgaaattggctctttagcttgtgtttctaatttttctttttcttcttttttcctttttgcaaaaacccaaaatattttagctcctactcagactgttactctggtgacacaacctgtggttactaaggaaactgccatctccaaactagaaatgccatcttccttgatgttggaggtacctgctctggcagatttcaaccgggcttggacagaacttaccgactggctttctctgcttgatcaagttataaaatcacagagggtgatggtgggtgaccttgaggatatcaacgagatgatcatcaagcagaaggtatgagaaaaaatgataaaagttggcagaagtttttctttaaaatgaagattttccaccaatcactttactctcctagaccatttcccaccagttcttaggcaactgtttctctctcagcaaacacattactctcactattcagcctaagtataatcaaggatataaattaatgcaaataacaaaagtagccatacattaaaaaggaaatatacaaaaaaaaaaaaaaaaaaaagcagaaaccttacaagaatagttgtctcagttaaatttactaaacaacctggtattttaaaaatctattttataccaaataagtcactcaactgagctatttacatttaaactgtttgttttggactacgcagcccaacatattgcagaatcaaatataatagtctgggaattgattattatccactcttctaagttgtctgtgccaatttgccttctccaatgataaggataattgaaagagagctataacttaaaaagagaaaagtaacaaaacataagatatttaaaattaccctagatcttaaagttggcatttatgcaatgccatgttcaaatgaacatgtttttaatacaaatagtgcatttttcagcctcagtgtaatccatttggtaaaattatgacatcaactagaaacattagaatacattgatgtaaatatggtttacctagctagatcaaatatactatatatcttttatatttgtgaatggttaagaaaaataatgttggaattgttatacattaaagttttttcacttgtaacagctttcaagcctttctaaagaaatacaaagttgtgctgaaggtatttaggtattaaagtactaccttttgaaaaaacaagaagtgaggcagacagagtaaggggaatttctttgtaaaataaacttcaccaattccataggaataaaagtaatttgatagtaaacaacctgcatttaaaggccttgagcttgaatacagaagacctgaattcagtgccatttgcaaatgatgattgtggtcaagccatctctggatcttcgtttcctattctgagtacagagcatacagagtacacattcacattcacaatatagttatggatatggatgtatataaatatatgtaaatactacatatatgtacctaaaatttgttttacttctgctttaaaaaaagtaattatagccacatttttcagaaaaagtaactgaggctcatagatgtcaaattcccagtaagtagcagaacaaggattcaaatccaagtccatttgattcctaagctt

SEQ ID NO: 238 - DNA, 2000 bp, Homo sapiens
gatctggaactgtggccatggtgtgaaggccatccacaaggtggaagctttgagggggagccgattagccatggacagttgtcattcagtagggtcacctgtgccccagcgaagggggatgggccgggaaggcagaggccaggcacctgcccccagcaggggcagaggctgtgggcagccgggaggctcccagaggctccgacagaatgggagtggggttgagcccacccctcactgcagcccaggaacctgagcccagagggggccacccaccttccccaggcagggaggcccggcccccagggagatgggggggatgggggaggagaagggcctgcccccacccggcagcctcaggaggggcagctcgggcgggatatggaaagaggccacagcagtgagcagagacacagaggaggaaggggccctgagctggggagacccccacggggtagggcgtgggggccacgggcccacctcctccccatctcctctgtctccctgtctctgtctctctctccctcccccaccctctccccagtcctaccccctcctcacccctcctcccccagcactgcctctgtcactctcgcccacgtggatgtggaggaagagggggcgggagcaaggggcgggcaccctcccttcaacctgacctgggacagtttcccttccgctcacctccgcctgagcagtggagaaggcggcactctggtggggctgctccaggcatgcagatcccacaggcgccctggccagtcgtctgggcggtgctacaactgggctggcggccaggatggttcttaggtaggtggggtcggcggtcaggtgtcccagagccaggggtctggagggaccttccaccctcagtccctggcaggtcggggggtgctgaggcgggcctggccctggcagcccaggggtcccggagcgaggggtctggagggacctttcactctcagtccctggcaggtcggggggtgctgtggcaggcccagccttggcccccagctctgccccttaccctgagctgtgtggctttgggcagctcgaactcctgggttcctctctgggccccaactcctcccctggcccaagtcccctctttgctcctgggcaggcaggacctctgtcccctctcagccggtccttggggctgcgtgtttctgtagaatgacgggtcaggctggccagaaccccaaaccttggccgtggggagtctgcgtggcggctctgccttgcccaggcatccttggtcctcactcgagttttcctaaggatgggatgagccccatgtgggactaaccttggctttacgacgtcaaagtttagatgagctggtgatatttttctcattatatccaaagtgtacctgttcgagtgaggacagttcttctgtctccaggatccctcctgggtggggattgtgcccgcctgggtctctgcccagattccagggctctccccgagccctgttcagaccatccgtgggggaggccttggccttactctcccggatcgaggagagagggagcctcttcctgggctgcccgtgaccctgggccctctgtgtacactgtgaccacagcccgctcctggaccctctgtgcccggctggccctctgtgcccagccagcctgcacctggggatgccaaggcctggggagggtggtttcacccaggccaagcctaagacagtccctctgggccctgctgggtaccggggtgtgacaccactgggaggacaagatgaggggcacccctggggccgccctgacacccccttgaggctcctgccccgggggtcctggtgccccttcactgtggcaggcgactgggggttccccacctcggcccctctcccggggcctgctccccggcacctgaggcagcatccttgtcagggccgtgccttcctgcctcagcgccacctcttaaggttggcccgtgggtcactcaggactcacaactggagattctgggcaaaaggcaaagagcaa

SEQ ID NO: 239 - DNA, 831 bp, Homo sapiens
cactgtagctgtactaccttccatctcctcaacctattccaactatctgaatcatgtgcccttctctgtgaacctctatcataatacttgtcacactgtattgtaattgtctcttttactttcccttgtatcttttgtgcatagcagagtacctgaaacaggaagtattttaaatattttgaatcaaatgagttaatagaatctttacaaataagaatatacacttctgcttaggatgataattggaggcaagtgaatcctgagcgtgatttgataatgacctaataatgatgggttttatttccagacttcacttctaatgatgattatgggagaactggagccttcagagggtaaaattaagcacagtggaagaatttcattctgttctcagttttcctggattatgcctggcaccattaaagaaaatatcatctttggtgtttcctatgatgaatatagatacagaagcgtcatcaaagcatgccaactagaagaggtaagaaactatgtgaaaactttttgattatgcatatgaacccttcacactacccaaattatatatttggctccatattcaatcggttagtctacatatatttatgtttcctctatgggtaagctactgtgaatggatcaattaataaaacacatgacctatgctttaagaagcttgcaaacacatgaaataaatgcaatttattttttaaataatgggttcatttgatcacaataaatgcattttatgaaatggtgagaattttgttcactcattagtgagacaaacgtctcaatggttatttatatggcatgcatatagtgatatgtggt

SEQ ID NO: 240 - DNA, 1100 bp, Homo sapiens
tatgatcctttgagagcctttagccgccgcagaacagcagtctggctatttagatagaacaacttgattttaagataaaagaactgtctatgtagcatttatgcatttttcttaagcgtcgatggaggagtttgtaaatgaagtacagttcattacgatacacgtctgcagtcaactggaattttcatgattgaattttgtaaggtattttgaaataatttttcatataaaggtgagtttgtattaaaaggtactggtggagtatttgatagtgtattaaccttatgtgtgacatgttctaatatagtcacattttcattatttttattataaggcctgctgaaaatgactgaatataaacttgtggtagttggagctggtggcgtaggcaagagtgccttgacgatacagctaattcagaatcattttgtggacgaatatgatccaacaatagaggtaaatcttgttttaatatgcatattactggtgcaggaccattctttgatacagataaaggtttctctgaccattttcatgagtacttattacaagataattatgctgaaagttaagttatctgaaatgtaccttgggtttcaagttatatgtaaccattaatatgggaactttactttccttgggagtatgtcagggtccatgatgttcactctctgtgcattttgattggaagtgtatttcagagtttcgtgagagggtagaaatttgtatcctatctggacctaaaagacaatctttttattgtaacttttatttttatgggtttcttggtattgtgacatcatatgtaaaggttagatttaattgtactagtgaaatataattgtttgatggttgatttttttaaacttcatcagcagtattttcctatcttcttctcaacattagagaacctacaactaccggataaattttacaaaatgaattatttgcctaaggtgtggtttatataaaggtactattaccaactttacctttgctttgttgtcatttttaaatttactcaaggaaatactaggatttaaaaaaaaattccttgagtaaatttaaattgttatcatgtttttgaggattattttcag

SEQ ID NO: 241 - DNA, 10 bp, Artificial Sequence
  Description of Artificial Sequence Synthetic oligonucleotide
tgagaatccg

SEQ ID NO: 242 - protein, 4 aa, Artificial Sequence
  Description of Artificial Sequence Synthetic peptide
  See specification as filed for detailed description of substitutions and preferred embodiments
GGGS

SEQ ID NO: 243 - protein, 5 aa, Artificial Sequence
  Description of Artificial Sequence Synthetic peptide
NSGDP

SEQ ID NO: 244 - protein, 48 aa, Artificial Sequence
  Description of Artificial Sequence Synthetic polypeptide
  SITE (1)..(48): This sequence may encompass 1-8 “Gly Gly Gly Gly Gly Ser” repeating units
  See specification as filed for detailed description of substitutions and preferred embodiments
GGGGGSGGGGGSGGGGGSGGGGGSGGGGGSGGGGGSGGGGGSGGGGGS

SEQ ID NO: 245 - DNA, 9 bp, Artificial Sequence
  Description of Artificial Sequence Synthetic oligonucleotide
ttagactct

SEQ ID NO: 246 - protein, 6 aa, Homo sapiens
NIIFGV

(19) United States
(12) Patent Application Publication
(10) Pub. No.: US 2024/0287547 A1
(43) Pub. Date: Aug. 29, 2024

(54) GENETIC MODIFICATION

(71) Applicant: Peter Biotherapeutics, Inc., Boston, MA (US)

(72) Inventors: Chenzhong Kuang, Lexington, MA (US); Yan Xiao, Lexington, MA (US); Dirk Herman Antonius Hondmann, East Sandwich, MA (US)

(21) Appl. No.: 18/001,340

(22) PCT Filed: Jun. 11, 2021
(86) PCT No.: PCT/US21/37113

§ 371 Date: Dec. 9, 2022

Related U.S. Application Data
(60) Provisional application No. 63/038,620, filed on Jun. 12, 2020.
(60) Provisional application No. 63/116,492, filed on Nov. 20, 2020.

(51) Int. Cl. C12N 15/90; C12N 9/22; C12N 15/11; C12Q 1/6874

(52) U.S. Cl. CPC C12N 15/90; C12N 9/22; C12N 15/11; C12Q 1/6874

(57) ABSTRACT

The present disclosure provides technologies for genetic modification without a need for introduction of one or more breaks into any genetic material being modified.

### CROSS-REFERENCE TO RELATED APPLICATIONS

The present application claims priority to each of U.S. Provisional patent application No. 63/038,620, filed on Jun. 12, 2020 and U.S. Provisional patent application No. 63/116,492, filed on Nov. 20, 2020, the entire disclosure of each of which is incorporated herein by reference.

### BACKGROUND

Gene editing and genome engineering hold great promise for the study of gene function and for the creation of new therapies for human diseases. There is a need for a greater variety of versatile method that can perform a wide variety of gene and/or genome conversions, which may be used to treat human disease.

### SEQUENCE LISTING

The instant application contains a Sequence Listing which has been submitted electronically in ASCII format and is hereby incorporated by reference in its entirety. Said ASCII copy, created on Jun. 11, 2021, is named 2013051-0005_SL.txt and is 363,811 bytes in size.

### SUMMARY

The present disclosure provides technologies (e.g., systems, compositions, methods, etc.) for modification of a polynucleotide. In some embodiments, the polynucleotide is or comprises DNA. In some embodiments, the polynucleotide is or comprises RNA (e.g., mRNA). In some embodiments, the modification is achieved via a system comprising one or more agents, e.g., an agent comprising one or more nucleotide binding elements and, optionally, an element comprising a nucleotide sequence used, in some way, to modify (e.g., via substitution, addition, deletion, etc.) one or more nucleotides at a target site. In some embodiments, the modification is achieved using a system comprising one or more agents that in some way modifies a process (e.g., transcription) at a target site.

In some embodiments, the present disclosure provides technologies to achieve genetic modification without a need to introduce one or more breaks into a target where a modification will occur. In some embodiments, the present disclosure provides technologies to achieve programmed gene regulation.

For example, the present disclosure provides, among other things, technologies by which a polymeric modification agent, for example, a DLR molecule induces a genetic modification when a single strand DNA donor template is present without need for DNA backbone breakages (see, e.g., FIGS. 1-5). In some embodiments, the present disclosure provides technologies by which a polymeric modification agent modifies one or more processes (e.g., transcription). In some embodiments, the present disclosure provides technologies where, for example, a DLR molecule is used for programmed gene regulation. In some such embodiments, such DLR molecules can regulate gene activity (e.g., suppress transcription) without a sequence modification polynucleotide.

In some embodiments, the present disclosure provides a polymeric modification agent comprising a structure represented by: D-L-R, wherein the D element is or comprises a sequence-specific binding element; the L element is optional and is or comprises a linker element; and the R element is or comprises a binding element that is optionally sequence-specific.

In some embodiments, a D element binds to a single strand on a first polynucleotide. In some embodiments, an R element binds to a single strand on a second polynucleotide. In some embodiments, each of a first and second polynucleotides may be part of the same or different molecules.

In some embodiments, the present disclosure provides a polymeric modification agent having a structure: D-L-R, comprising at least one D element, at least two R elements, and, optionally, two or more L elements, wherein: D is or comprises a sequence-specific DNA binding element that binds to one strand; L is or comprises an optional linker element; and R is or comprises a DNA binding element that binds to a strand opposite to which a D element is bound.

In some embodiments, the present disclosure provides a polymeric modification agent having a structure: D-L-R, comprising at least one D element, an optional L element between the D and R elements, and a least one R element. In some embodiments, a polymeric modification agent comprises at least two R elements, and, optionally, two or more L elements. In some embodiments, a D element is or comprises a sequence-specific DNA binding element that binds to one strand of a polynucleotide, L is or comprises an optional linker element, and R is or comprises a DNA binding element that binds to a strand opposite the strand to which a D element is bound.

In some embodiments, the present disclosure provides a polymeric modification agent comprising a structure represented by: D-L-Rn, wherein the D element is or comprises a sequence-specific binding element; the L element is optional and is or comprises a linker element; the R element is or comprises a binding element that is optionally sequence-specific, and n equals 1, 2, or 3.

In some embodiments, a polymeric modification agent comprises at least two R elements (e.g., at least 3, 4, 5, 6, 7, 8, 9, 10 or more R elements).

In some embodiments, the present disclosure provides a polymeric modification agent having a structure: D-L-R, comprising at least one D element, at least two R elements, and, optionally, at least one L element, wherein: D is or comprises a sequence-specific DNA binding element that binds to one strand; L is or comprises an optional linker element; and R is or comprises a DNA binding element that binds to a strand opposite to which a D element is bound.

In some embodiments, a polymeric modification agent does itself modify a target site or target sequence and/or does not cause modification of a non-target site.

In some embodiments, no component of a polymeric modification agent of the present disclosure acts primarily as a nuclease.

In some embodiments, the present disclosure provides a D element which is or comprises a polypeptide. In some embodiments, such a polypeptide is between 80 and 10,000 amino acids in length or 8 kD and 1,000 kD in size. In some embodiments, a D element has or comprises a sequence that has or comprises a sequence that is at least 50% identical to a sequence selected from SEQ ID NOS 2, 3, 5, 7, 9, 11, 12, 161, 162, 174, 175, 181, 184, 187, 188, 189, 196, 197, 219, 222, 225, or 226. In some embodiments, a D element is or comprises a polynucleotide. In some such embodiments, such a polynucleotide is between 20 and 50,000 nucleotides in length.

In some embodiments, a D element is or comprises a catalytically inactive protein, such as a catalytically inactive Cas protein (e.g., dCas9).

In some embodiments, a D element comprises one or more nucleotides that bind at or near a landing site adjacent to a target site. In some embodiments, a D element comprises one or more amino acids that bind at or near a landing site adjacent to a target site. In some embodiments, a D element has a binding affinity with a dissociation constant of 10E-6 or lower for at least one target site.

In some embodiments, the present disclosure provides a combination comprising a polymeric modification agent as described herein and a sequence modification polynucleotide. In some such embodiments, a polynucleotide comprises more than one chain of polynucleotides. In some embodiments, a polymeric modification agent of the present disclosure comprises a D element that has or comprises a sequence that is at least 50% identical to a sequence selected from SEQ ID NOS 91, 92, 93, 94, 95, 96, 97, 230, 231, 232, 233, 234, or 235.

In some embodiments, the present disclosure provides an L element that is or comprises a polypeptide. In some embodiments, an L element is or comprises a polypeptide between 2 and 100 amino acids in length or 0.2 kD and 10 kD in size. In some embodiments, an L element has or comprises a sequence that is at least 50% identical to a sequence selected from SEQ ID NOS 1, 13, or 14. In some embodiments, an L element is or comprises a polynucleotide. In some such embodiments, such a polynucleotide is between 2 and 500 nucleic acids in length. In some such embodiments, a polynucleotide comprises more than one chain of polynucleotides. In some embodiments, a polymeric modification agent of the present disclosure comprises an L element that has or comprises a sequence that is at least 50% identical to a sequence selected from SEQ ID NOS 98, 99, or 100.

In some embodiments, the present disclosure provides an R element that is or comprises a polypeptide. In some embodiments, an R element is or comprises a polypeptide between 10 and 50,000 amino acids in length or 1 kD and 5,000 kD in size. In some embodiments, an R element has or comprises a sequence that is at least 50% identical to a sequence selected from SEQ ID NOS 19, 81, 84, 101-128, 208, 210, 212, 214, or 216. In some embodiments, an R element is or comprises a polynucleotide. In some such embodiments, the polynucleotide is between 2 and 50,000 nucleic acids in length. In some embodiments, an R element has or comprises a sequence that is at least 50% identical to a sequence selected from SEQ ID NOS 20, 85, 129-156, 207, 209, 211, 213, or 215. In some embodiments, a R element is or comprises a polynucleotide which polynucleotide comprises a single polynucleotide chain; in some embodiments, the polynucleotide comprises more than one chain of polynucleotides. In some embodiments, an R element has a binding affinity with a dissociation constant of 10E-3 or lower for at least one target site.

Among other things, the present disclosure provides a method comprising a step of contacting a cell comprising DNA with a combination comprising (i) a polymeric modification agent of the present disclosure; and (ii) a sequence modification polynucleotide, wherein: (a) the DNA includes at least one target site; (b) the D element of the polymeric modification agent associates with a landing site adjacent to the target site that includes at least one target sequence; and (c) the sequence modification polynucleotide: (i) binds specifically to one strand of the DNA at the target site; and (ii) has a mismatch or other DNA sequence difference relative to the target site, so that usage of the sequence modification polynucleotide incorporates the sequence modification into a complement of the one strand. In some embodiments, a polymeric modification agent does not directly catalyze single and/or double-stranded DNA breaks. In some embodiments, a target site is an error site.

In some embodiments, the present disclosure provides, among other things, a method comprising a step of contacting DNA with a combination comprising (i) a polymeric modification agent as provided herein; and (ii) a sequence modification polynucleotide, wherein: (a) the DNA includes at least one target sequence; (b) the D element of the agent binds to a landing site adjacent to a target site that includes at least one target sequence; and (c) the sequence modification polynucleotide: (i) binds specifically to one strand of the DNA at the target site; and (ii) has a DNA sequence difference relative to the target sequence. In some embodiments, use of a sequence modification polynucleotide results in a change in a polynucleotide sequence at a target site relative to before use of the sequence modification polynucleotide.

In some embodiments, the present disclosure provides a method comprising contacting a cell comprising DNA with a polymeric modification agent wherein (a) the DNA includes at least one target site; (b) the D element of the polymeric modification agent associates with a landing site adjacent to the target site that includes at least one target sequence; (c) the one, two, or three R-elements binds to one strand of the DNA at the target site; and there is a reduced mRNA level of a target after the contacting relative to a cell that is not contacted with the polymeric modification agent.

In some embodiments, DNA is actively replicating. In some embodiments, contacting occurs within the context of a DNA replication fork. In some embodiments, contacting results in a reduction in speed of DNA replication. In some embodiments, contacting results in a reduction in speed of DNA replication within the vicinity of the target site.

In some embodiments, DNA is being actively transcribed. In some embodiments, transcription activity of a target is reduced after a cell comprising a target is contacted with a polymeric modification agent.

In some embodiments the step of contacting comprises contacting within a cell.

In some embodiments, a cell is a postmitotic cell.

In some embodiments, contacting comprises contacting a population of cells. In some embodiments, a population of cells is or comprises a tissue. In some embodiments, a population of cells is or comprises an organ. In some embodiments, a population of cells is or comprises a tumor. In some embodiments, a tumor is or comprises a pancreatic tumor, colon tumor or lung tumor. In some embodiments, a population of cells is or comprises a specific cell lineage. In some embodiments, a specific cell lineage is or comprises neural cells. In some embodiments, a specific cell lineage is or comprises neuronal cells.

In some embodiments, contacting occurs in vivo.

In some embodiments, contacting is performed ex vivo or in vitro.

In some embodiments, contacting is performed ex vivo or in vitro, resulting in a population of cells with at least one modified DNA sequence relative to the population of cells prior to the contacting. In some embodiments, at least a portion of the population of cells is administered to a subject in need thereof.

In some embodiments, contacting comprises contacting with a system that includes a DNA polymerase or any other factors associated with DNA modification and repair, such as helicases, ligases, recombinases, repair scaffold proteins, single strand DNA binding proteins, mismatch repair proteins or any other protein that can be associated with DNA modification processes.

In some embodiments, contacting further comprises use of an enhancing agent and/or an inhibiting agent. In some embodiments, use of an enhancing and/or inhibiting agent enhances recombination events in DNA contacted with a combination of a polymeric modification agent and sequence modification polynucleotide, but the enhancing agent and/or inhibiting agent itself does not contact the DNA being contacted by the combination.

In some embodiments, an enhancing agent and/or inhibiting agent is or comprises RNAi activity. In some embodiments, an enhancing agent and/or inhibiting agent inhibits one or more of CDC45 or XRCC1. In some embodiments, incorporation of a sequence modification into a complement of a strand of DNA to which a D element is bound occurs at a frequency of two to ten times greater than a frequency of incorporation of the sequence modification into the complement of the one strand that occurs in the absence of the enhancing agent and/or inhibiting agent.

In some embodiments, incorporation of a sequence modification into a complement of one strand of DNA occurs concomitant with, or subsequent to, a reduction in rate of replication fork activity in the DNA.

In some embodiments, contacting is achieved by administration of at least one polymeric modification agent in accordance with the present disclosure and, optionally, at least one sequence modification polynucleotide by at least one of intravenous, parenchymal, intracranial, intracerebroventricular, intrathecal, or parenteral administration.

In some embodiments, contacting occurs in a subject in need thereof. In some embodiments, a subject is a mammal. In some embodiments, a mammal is a non-human primate. In some embodiments, a mammal is a human. In some embodiments, a human is an adult human. In some embodiments, a human is a fetal, infant, child, or adolescent human.

In some embodiments of the present disclosure, a single target site and/or target sequence is modified. In some embodiments, at least one target site and/or target sequence is modified. In some embodiments, at least two target sites and/or sequences are modified. In some embodiments, at least two target sites and/or sequences are associated with different genes; in some such embodiments, different genes are located on the same chromosome and in some embodiments, different genes are located on different chromosomes. In some embodiments, at least two target sites and/or sequences are associated with the same gene. In some embodiments, a modification is a disruption and/or dissociation of a polymerase (e.g., an RNA polymerase) from a polynucleotide (e.g., DNA) strand.

In some embodiments of the present disclosure, methods comprising contacting include contacting with at least two sets of compositions, wherein each composition comprises a polymeric modification agent in accordance with the present disclosure and a sequence modification polynucleotide. In some embodiments, contacting with at least two sets of compositions as described herein comprises sequential contacting with at least a first set followed by at least a second set. In some embodiments, contacting at least two sets of compositions as described herein comprises simultaneous contacting with at least a first set and a second set.

In some embodiments, a sequence modification polynucleotide of the present disclosure is or comprises a deletion, substitution, or insertion, relative to the target sequence. In some embodiments, a sequence modification polynucleotide has a single nucleotide difference relative to that of a target sequence. In some embodiments, a sequence of a sequence modification polynucleotide comprises a plurality of differences relative to that of the target site. In some embodiments, a sequence modification polynucleotide is between 10 and 20,000 nucleotides in length. In some embodiments, a sequence modification polynucleotide is more than 2,000 nucleotides in length. In some embodiments, a sequence modification polynucleotide is or comprises a sequence with at least 50% identity to a sequence selected from SEQ ID NOS 22, 23, and 29-33.

In some embodiments, a sequence modification polynucleotide comprises a sequence that is capable of being incorporated into a copy of a human ApoE gene during DNA replication or DNA synthesis (i.e., a copy of a gene sequence that is produced as a result of endogenous DNA replication machinery in a cell, i.e., an endogenous nucleic acid sequence (e.g., gene, promoter, enhancer, etc. and combinations thereof)). In some embodiments, an ApoE gene has sequence that is at least 70% identical to the sequence set forth in SEQ ID NO: 157.

In some embodiments, a sequence modification polynucleotide comprises a sequence that is capable of being incorporated into a copy of a human BCL11A gene during DNA replication or DNA synthesis (i.e., a copy of a gene sequence that is produced as a result of endogenous DNA replication machinery in a cell, i.e., an endogenous nucleic acid sequence (e.g., gene, promoter, enhancer, etc. and combinations thereof)). In some embodiments, a BCL11A sequence modification polynucleotide has sequence that is at least 70%, 75%, 80%, 85%, 90%, 95%, 96%, 97%, 98%, 99% or more identical to the sequence set forth in SEQ ID NO: 163. In some embodiments, a BCL11A gene has sequence that is at least 70%, 75%, 80%, 85%, 90%, 95%, 96%, 97%, 98%, 99% or more identical to the sequence set forth in SEQ ID NO: 236.

In some embodiments, a sequence modification polynucleotide comprises a sequence that is capable of being incorporated into a copy of a human DMD gene, (dystrophin) during DNA replication or DNA synthesis (i.e., a copy of a gene sequence that is produced as a result of endogenous DNA replication machinery in a cell, i.e., an endogenous nucleic acid sequence (e.g., gene, promoter, enhancer, etc. and combinations thereof)). In some embodiments, a DMD sequence modification polynucleotide has sequence that is at least 70%, 75%, 80%, 85%, 90%, 95%, 96%, 97%, 98%, 99% or more identical to the sequence set forth in SEQ ID NO: 176. In some embodiments, a DMD (dystrophin) gene has sequence that is at least 70%, 75%, 80%, 85%, 90%, 95%, 96%, 97%, 98%, 99% or more identical to the sequence set forth in SEQ ID NO: 237.

In some embodiments, a sequence modification polynucleotide comprises a sequence that is capable of being incorporated into a copy of a human PDCD-1 gene during DNA replication or DNA synthesis (i.e., a copy of a gene sequence that is produced as a result of endogenous DNA replication machinery in a cell, i.e., an endogenous nucleic acid sequence (e.g., gene, promoter, enhancer, etc. and combinations thereof)). In some embodiments, a PDCD-1 sequence modification polynucleotide has sequence that is at least 70%, 75%, 80%, 85%, 90%, 95%, 96%, 97%, 98%, 99% or more identical to the sequence set forth in SEQ ID NO: 190. In some embodiments, a PDCD-1 gene has sequence that is at least 70%, 75%, 80%, 85%, 90%, 95%, 96%, 97%, 98%, 99% or more identical to the sequence set forth in SEQ ID NO: 238. In some embodiments, a sequence modification polynucleotide comprises a sequence that is capable of being incorporated into a copy of a human CFTR gene during DNA replication or DNA synthesis (i.e., a copy of a gene sequence that is produced as a result of endogenous DNA replication machinery in a cell, i.e., an endogenous nucleic acid sequence (e.g., gene, promoter, enhancer, etc. and combinations thereof)). In some embodiments, a CFTR sequence modification polynucleotide has sequence that is at least 70%, 75%, 80%, 85%, 90%, 95%, 96%, 97%, 98%, 99% or more identical to the sequence set forth in SEQ ID NO: 198. In some embodiments, a CFTR gene has sequence that is at least 70%, 75%, 80%, 85%, 90%, 95%, 96%, 97%, 98%, 99% or more identical to the sequence set forth in SEQ ID NO: 239.

In some embodiments, a sequence modification polynucleotide comprises a sequence that is capable of being incorporated into a copy of a human KRAS gene during DNA replication or DNA synthesis (i.e., a copy of a gene sequence that is produced as a result of endogenous DNA replication machinery in a cell, i.e., an endogenous nucleic acid sequence (e.g., gene, promoter, enhancer, etc. and combinations thereof)). In some embodiments, a KRAS targeting sequence has sequence that is at least 70% identical to the sequence set forth in SEQ ID NO: 226. In some embodiments, a KRAS sequence modification polynucleotide has sequence that is at least 70%, 75%, 80%, 85%, 90%, 95%, 96%, 97%, 98%, 99% or more identical to the sequence set forth in SEQ ID NO: 227. In some embodiments, a KRAS gene has sequence that is at least 70%, 75%, 80%, 85%, 90%, 95%, 96%, 97%, 98%, 99% or more identical to the sequence set forth in SEQ ID NO: 240.

In some embodiments, a sequence modification polynucleotide comprises a sequence that is capable of being incorporated into an exogenous sequence, e.g., an exogenous gene that has been incorporated into genetic material, e.g., of host genetic material, for example, a viral genome, gene and/or components thereof.

In some embodiments, methods as provided herein further comprise administration of at least one additional agent. In some embodiments, at least one additional agent is or comprises an agent that induces DNA replication. In some embodiments, at least one additional agent is or comprises an agent that induces DNA breakage.

In some embodiments, the present disclosure provides, among other things, a combination comprising at least one polymeric modification agent as disclosed herein; and a sequence modification polynucleotide. In some such embodiments, the present disclosure provides at least two such compositions.

In some embodiments, the present disclosure provides a method comprising: contacting a cell with a combination comprising (i) a polymeric modification agent as provided herein; and (ii) a sequence modification polynucleotide.

In some embodiments, the present disclosure provides a method comprising contacting a cell with a polymeric modification agent as described herein.

In some embodiments, the present disclosure provides kits comprising at least one agent or composition as described herein. In some embodiments, a kit of the present disclosure further provides an agent that is or comprises an agent that induces DNA replication or induces DNA strand breakage.

In some embodiments, the present disclosure provides a method of characterizing one or more elements of a polymeric modification agent in accordance with the present disclosure, which method comprises measuring one or more of binding efficiency, binding affinity, sequence modification efficiency, and stability of the at least one element.

In some embodiments, the present disclosure provides a method of characterizing a polymeric modification agent as provided herein, comprising measuring an mRNA level of a target in presence or absence of the polymeric modification agent.

### BRIEF DESCRIPTION OF THE DRAWING

FIG. 1 is a schematic of representative events that may occur during DNA replication.

FIG. 2 is a representative schematic showing an exemplary blocking agent and an exemplary donor template. In this schematic, the exemplary blocking agent binds to double-stranded DNA strongly enough to slow down or stall a replication fork during DNA replication, and the exemplary donor template anneals with one of the two strands of separated DNA within replication fork.

FIGS. 3A, 3B, and 3C show an exemplary enabling DNA conversion at an installing replication fork. Panels 3A and 3B show an example of how mismatch repair and DNA replication may be manipulated to edit DNA in the presence of a blocking agent. Panel 3C illustrates activity at a replication fork restarting after dissociation of a blocking agent.

FIGS. 4A, 4B, and 4C show exemplary DNA repair mechanisms. Panel 4A illustrates a strand of DNA to be repaired (dashed and angled line). Panel 4B shows a mismatch repair approach. Panel 4C shows a base excision repair approach.

FIG. 5 is a schematic showing an exemplary factor involved in replication restart.

FIG. 6 is a schematic of a DLR molecule.

FIG. 7 is an exemplary schematic of a DLR molecule, with a “D” element comprising a zinc finger domain.

FIGS. 8A, 8B, 8C, 8D, and 8E illustrate certain steps as they may occur via DLR-mediated genetic conversion. Panel 8A shows a DLR molecule binding at a specific target site in a genome. Panel 8B shows a DLR molecule stalling replication fork progression. Panel 8C shows a donor template that has a desired DNA modification annealing to its complementary DNA strand. Panel 8D shows creation of a mismatch mutation, which can integrate into a genome. Panel 8E shows an integrated DNA modification introduced by steps including those shown in Panels 8A-8D.

FIG. 9 illustrates an exemplary assay to measure gene conversion.

FIG. 10 demonstrates generation of an exemplary reporter gene in an exemplary cell line.

FIGS. 11A, 11B, and 11C show an exemplary targeting and conversion strategy that restores in-frame expression of EGFP by correcting two point mutations in EGFPDP2. Panel 11A shows DNA sequences of the target, template, and wild-type gene. Panel 11B shows a frameshift mutation and early termination of translation for target as compared with the wild-type gene. Panel 11C illustrates double stranded DNA targeting by the DLR molecule used for editing.

FIGS. 12A and 12B demonstrate successful gene conversion (i.e., gene editing) at a cellular level using EGFPDP2 (a non-fluorescing variant) and EGFP. Panel 12A shows absence of fluorescent signal in EGFPDP2 cells. Panel 12B shows presence of green fluorescent signal after editing of EGFPDP2 using an exemplary DLR molecule.

FIGS. 13A, 13B, and 13C demonstrate successful gene editing using an exemplary DLR molecule. Panel 13A shows a sequence alignment of EGFPDP2 (a non-fluorescing variant) and EGFP, indicating a “G” insertion and a C→G conversion after editing. Panel 13B is a chromatogram from Sanger sequencing of EGFPDP2. Panel 13C is a Sanger sequencing chromatogram of targeted and repaired EGFP2 genes, with positions of gene edits indicated.

FIGS. 14A and 14B show exemplary insertion and deletion (“indel”) analysis by next generation sequencing of untargeted (“EGFPDP2”), non-edited (“Negative Clone”), and edited (“Positive Clone”) cells. Panel 14A shows an overview of indels at each target site in EGFPDP2 and panel 14B shows an enlarged view of the indicated region in panel 14A.

FIGS. 15A, 15B, and 15C show an exemplary single nucleotide polymorphism (SNP) analysis by next generation sequencing of untargeted (“EGFPDP2”), non-edited (“Negative Clone”), and edited (“Positive Clone”) cells. Panel 15A shows an overview of SNPs at each target site in EGFPDP2 and panel 15B shows an enlarged view of the indicated region in panel 15A. Panel 15C shows percent distribution of genotypes at the targeted position in untargeted, non-edited, and edited cells.

FIG. 16 shows total reads as well as genotypes by next generation sequencing of untargeted (“EGFPDP2”), non-edited (“Negative Clone”), and edited (“Positive Clone”) cells.

FIG. 17 illustrates targeting and editing at codon 112 of human endogenous ApoE, as well as ddPCR detection of T→C conversion in HEK293 cells.

FIG. 18 demonstrates T→C genetic conversion at codon 112 of human ApoE by ddPCR analysis of dots representing droplets, containing indicated C or T alleles.

FIGS. 19A and 19B show editing efficiency at codon 112 site of ApoE in HEK293 cells. Panel A shows droplet events at each channel designed to detect C or T alleles. Panel B shows genetic T→C editing frequencies.

FIGS. 20A and 20B show Single Nucleotide Polymorphisms (SNP) analysis by next generation sequencing between untargeted, and edited cells. Panel A shows overviews of SNPs at each position of the targeting region of codon 112 site of human ApoE. Panel B shows an enlarged, trimmed view in the region adjacent to codon 112 site of human ApoE.

FIG. 21 shows insertion and deletion (Indels) analysis by next generation sequencing between untargeted and edited cells.

FIG. 22 illustrates isolated single clones for genotypic and phenotypic characterization of T→C genetic editing at codon 112 site of ApoE in HEK293 cells.

FIGS. 23A and 23B show an example of identification of single clone with a T→C conversion by ddPCR. Panel A shows ddPCR dot plots of positive controls as well as negative and positive clones for this genomic target. Panel B shows a ddPCR 2D-plot distribution of “C” and “T” genotypes at the target site.

FIG. 24 shows successful T→C conversion in single clones by Sanger sequencing.

FIG. 25 shows Single Nucleotide Polymorphism (SNP) analysis by next generation sequencing of exemplary positive or unconverted, negative clones after sequence modification.

FIG. 26 shows insertion and deletion (Indel) analysis by next generation sequencing of a positive clone and an unconverted negative clone.

FIG. 27 is an overview of circular sequencing for unbiased genome-wide on- and off-target sites analysis.

FIG. 28 shows an example of a molecular structure and interpretation of one sequence read from circular sequencing.

FIG. 29 is a DNA sequence alignment demonstrating on-target gene editing with no off-target site incidences.

FIG. 30 shows the results from circular sequencing for genome-wide on- and off-target site analysis.

FIG. 31 illustrates targeting and editing at codon 158 of human endogenous ApoE, as well as a schematic of droplet digital PCR-based (ddPCR) detection of C→T conversion in HEK293 cells.

FIG. 32 shows an example of successful genetic T→C conversion after targeting and editing at codon 158 of ApoE in HEK293 cells by ddPCR.

FIG. 33 shows an example of codon 158 site editing frequency.

FIG. 34 shows an ApoE genotype in human U937 cells by Sanger sequencing.

FIG. 35 illustrates targeting and editing at codon 112 site of human endogenous ApoE, as well as a schematic of droplet digital PCR-based (ddPCR) detection of C→T conversion in U937 cells.

FIG. 36 illustrates experimental schematics of a timed delivery of a DLR molecule into human U937 cells for genome editing.

FIG. 37 shows analysis of a C→T genetic conversion at codon 112 of human ApoE in U937 cells by ddPCR analysis, representing droplets containing indicated C or T alleles.

FIG. 38 shows ApoE codon 112 site editing frequency in U937 cells.

FIG. 39 shows multiple amino acid sequence alignments of representative R elements based on a PD-(D/E)XK structural core fold.

FIG. 40 provides a table of targeting frequency analysis from multiple D-L-R constructs with deactivated critical sites for abolishment of DNA cleavage activity.

FIG. 41 shows representative results from ddPCR analysis for identification of positive cellular clones containing a T-to-C conversion at codon 112 of human ApoE in HEK293 cells.

FIGS. 42A, 42B, and 42C show multiple amino acid sequence alignment of exemplary DLR molecules with a variant hybrid PD-(D/E)XK core fold. Panel A shows multiple amino acid sequence alignments of functional R elements and naturally occurring nucleases to show inactivated critical sites in this PD-(D/E)XK core fold. Panel B shows an amino acid alignment of R elements of exemplary DLR molecules having multiple inactivated PD-(D/E)XK cores in their beta sheet 2-loop 2-beta sheet 3 regions. Panel C shows an amino acid sequence alignment of a set of R elements from exemplary DLR molecules having multiple inactivated PD-(D/E)XK cores in their loop 1 regions.

FIG. 43 provides a table of targeting frequency analysis from exemplary DLR molecules with an inactived PD-(D/E)XK core derived from naturally occurring nucleases.

FIGS. 44A and 44B show a schematic depicting an exemplary DLR molecule made from catalytically inactive Cas9 (dCas9). Panel A illustrates targeting and editing at EGFPDP2 gene by a DLR molecule with dCas9 as the D element. Panel B is a molecular structure of this dCas9-L-R chimera construct.

FIG. 45 shows that a dCas9-based DLR designed to target an EGFPDP2 mutant locus restores expression of functional EGFP.

FIG. 46 is a schematic of architecture of an exemplary DLR molecule comprising of a versatile R unit with sequence-specific DNA binding ability.

FIGS. 47A, 47B, and 47C show a schematic approach to targeting and editing a EGFPDP2 mutant gene by a dual zinc finger array. Panel A shows DNA sequences of EGFPDP2, ssODN template (i.e., sequence modification polynucleotide), and EGFP fixation aligned to show two mutations at this targeting site of EGFPDP2 and its repaired sequence. Panel B illustrates double stranded DNA targeting by a DLR molecule with dual non-cleavage zinc finger arrays. Panel C shows dual zinc arrays binding two recognizing sites of an EGFDP2 mutant locus on each strand of DNA.

FIGS. 48A and 48B show that EGFPDP2 is targeted and repaired by a non-cleavage, double zinc finger array-unit DLR. Panel A is a schematic illustrating an assay of genetic EGFPDP2→EGFP conversion using this DLR molecule with dual zinc finger arrays. Panel B shows how mutant EGFPDP2 was repaired to express functional EGFP.

FIG. 49 is a schematic representation outlining in situ analysis of protein interactions at DNA replication forks (SIRF) assay for analysis of DLR molecule proximity to replication forks.

FIG. 50 is an illustration of close proximity of a DLR molecule and a replication fork.

FIG. 51 illustrates experimental schematics of timed delivery of a DLR molecule as well as an RNAi with cell cycle synchronization in HEK293 cells for genome editing.

FIG. 52 shows ddPCR analysis to determine impact of reduction of specific factors by RNAi to inhibit CDC45 or XRCC1 on gene editing efficiency.

FIG. 53 shows editing frequency based on ddPCR droplet event numbers representing a T-to-C conversion at codon 112 of human ApoE in HEK293 cells. RNAi was used for inhibition of CDC45 and XRCC1, respectively

FIG. 54 shows ddPCR analysis to determine impact of reducting specific factors by RNAi to Inhibit CDC45 or MSH2 on gene editing efficiency.

FIG. 55 shows calculated editing frequency based on ddPCR droplet event numbers representing a T-to-C conversion at codon 112 of human ApoE in HEK293 cells. RNAi was used for inhibition of CDC45 and MSH2, respectively.

FIG. 56 is a schematic showing aspects of an exemplary targeting and editing strategy of an exemplary gene using a DLR molecule in accordance with the present disclosure. In this Figure, an enhancer within intron 2 of human BCL11A is targeted for editing.

FIG. 57 is a schematic that depicts ddPCR detection of TTATC→GAATTC conversion at an enhancer within intron 2 of human BCL11A in HEK293 cells.

FIGS. 58A and 58B demonstrate TTATC→GAATTC genetic conversion at an enhancer within intron 2 of human BCL11A gene by ddPCR analysis of dots representing droplets, containing indicated GAATTC (58A, top panel) or TTATC (58B, bottom panel) alleles.

FIGS. 59A and 59B show an exemplary single nucleotide polymorphism (SNP) analysis by next generation sequencing of untargeted and RITDM pb43-edited cells. FIG. 59A shows an overview of SNPs at each target site at an enhancer within intron 2 of human BCL11A gene.

FIG. 59B shows an enlarged view of the indicated region in 59A.

FIGS. 60A and 60B show exemplary insertion and deletion (“indel”) analysis by next generation sequencing of untargeted, and RITDM pb43 edited cells FIG. 60A shows an overview of indels at each target site in at enhancer within intron2 of human BCL11A gene.

FIG. 60B shows an enlarged view of the indicated region in 60A.

FIG. 61 shows overall indel frequencies at each nucleotide position at a target site in an enhancer within intron 2 of human BCL11A gene in untargeted and RITDM pb43 edited HEK293 cells.

FIG. 62 shows dual zinc arrays binding two recognizing sites of at an enhancer within intron 2 of human BCL11A gene on two strands of DNA.

FIG. 63 illustrates targeting and editing by RITDM with pb46 at an enhancer within intron 2 of human BCL11A gene, as well as a schematic of droplet digital PCR-based (ddPCR) detection of TTATC→GAATTC conversion in U937 cells.

FIGS. 64A and 64B demonstrate TTATC→GAATTC genetic conversion by RITDM with pb46 at enhancer within intron 2 of human BCL11A gene by ddPCR analysis of dots representing droplets, containing indicated GAATTC (64A, upper panel) or TTATC (64B, lower panel) alleles in U937 cells. Untargeted (i.e., negative control) cells are on the left side of each panel, and targeted and edited cells on the right, with edited and unedited cell genotypes separated by a solid line.

FIGS. 65A and 65B demonstrate successful gene editing using an exemplary DLR molecule. FIG. 65A is a chromatogram from Sanger sequencing of a “wild type” enhancer within intron 2 of human BCL11A gene with target sequence “TTATC” indicated. FIG. 65B is a Sanger sequencing chromatogram of RITDM edited enhancer within intron 2 of human BCL11A genes, with “GATTCC” genetic conversion indicated.

FIG. 66 shows detection of a TTATC→GAATTC genetic conversion at an enhancer within intron 2 of human BCL11A gene using restriction fragment length polymorphisms (RFLP) and results of an RFLP comparison between undigested and EcoRI digested amplicons from untargeted, and RITDM pb46 edited U937 pooled cells.

FIGS. 67A and 67B demonstrated successful gene editing using RITDM with pb46 at an enhancer within intron 2 of human BCL11A gene, measured by next generation sequencing. FIG. 67A shows frequencies of a TT→GA conversion by SNP analysis. FIG. 67B shows frequencies of a T insertion at a desired position by Indel analysis.

FIG. 68A illustrates a RITDM targeting and editing strategy in exon 51 of human dystrophin gene. FIG. 68B shows a schematic of a ddPCR detection strategy (“converted” vs “wild type” probes) used to detect “GA” 2-nucleotide insertion in mammalian cells.

FIGS. 69A and 69B show droplets from ddPCR analysis demonstrating presence of either edited (“GA” insertion; FIG. 69A, top panel) or wild-type (“TTATC” sequence, unedited; FIG. 69B, bottom panel) alleles.

FIGS. 70A and 70B demonstrate successful gene editing using an exemplary DLR molecule. FIG. 70A is a chromatogram from Sanger sequencing of “wild type” exon 51 of dystrophin with a nucleotide “C” as indicated. FIG. 70B is a Sanger sequencing chromatogram of RITDM-edited exon 51 of dystrophin with a “GA” 2-nucleotide insertion as indicated.

FIG. 71 shows an exemplary single nucleotide polymorphism (SNP) analysis by next generation sequencing of untargeted and RITDM pb49 edited cells at exon 51 of dystrophin gene.

FIG. 72 shows exemplary insertion and deletion (“indel”) analysis by next generation sequencing of untargeted and RITDM pb49 edited cells at exon 51 of dystrophin gene.

FIGS. 73A and 73B shows an indel length histogram as analyzed by next generation sequencing. FIG. 73A represents untargeted U937 cells; while FIG. 73B represents RITDM edited U937 cells, showing a large number of reads with a desired 2-nucleotide insertion after editing.

FIG. 74 illustrates results of overall editing efficiency and indel frequencies at exon 51 of dystrophin gene comparing untargeted and RITDM pb49 targeted cells.

FIGS. 75A, 75B, and 75C illustrates a RITDM targeting and editing strategy for editing of a region including a start codon ATG of human PDCD-1 gene. FIG. 75A illustrates targeting sites close to a start codon, ATG, of human PDCD-1 as well as recognition sites for designed DLR molecules. FIG. 75B demonstrates a designed sequence modification polynucleotide used to introduce a stop codon at a target site with an illustrative stop codon indicated. FIG. 75C illustrates ddPCR detection of a “CA→AATTCAT” conversion in human cells.

FIG. 76 demonstrates a “CA→AATTCAT” genetic conversion at human PDCD-1 gene by ddPCR analysis of dots representing droplets, containing indicated “CA” or “AATTCAT” sequences.

FIG. 77 shows overall editing frequencies of a RITDM introduction of a stop codon into a PDCD-1 gene for a negative control as well as three specially designed exemplary DLR molecules, as measured by ddPCR.

FIGS. 78A and 78B illustrates a RITDM targeting and editing strategy for editing of a region including codon F508 site of human CFTR gene as well as a detection method. FIG. 78A illustrates targeting sites close to codon F508 site of human CFTR gene as well as an exemplary RITDM editing strategy including a recognition site for a designed DLR molecule and an engineered sequence modification polynucleotide used to convert multiple nucleotide at a target site close to codon F508. FIG. 78B illustrates ddPCR detection of a “CTT→ATG” conversion in human cells.

FIG. 79 illustrates genetic and amino acid sequences of CFTR adjacent to codon F508 representing “normal” or “wild-type”, CFTR ΔF508, and predicted genetic conversion after RITDM editing.

FIGS. 80A and 80B demonstrate a “CTT→ATG” genetic conversion at human CFTR gene by ddPCR analysis. FIG. 80A shows analysis of a CTT→ATG genetic conversion at codon F508 of human CFTR in HEK293 cells by ddPCR analysis, representing droplets containing indicated CTT or ATG alleles. FIG. 80B shows overall editing frequencies of a RITDM editing at human CFTR gene in HEK293 cells, as measured by ddPCR.

FIGS. 81A and 81B depicts evidence demonstrating successful gene editing using RITDM with pb64 at F508 site of human CFTR gene, measured by next generation sequencing. FIG. 81A shows frequencies of a CTT→ATG conversion by SNP analysis between untargeted and targeted HEK293 cells. FIG. 81B shows a magnified view of depictions of frequencies of a CTT→ATG at a target site comparing untargeted and targeted HEK293 cells.

FIG. 82 shows exemplary insertion and deletion (“indel”) analysis by next generation sequencing of untargeted and RITDM pb64 edited cells at F508 site of human CFTR gene in HEK293 cells. FIG. 82A shows an indel length histogram as analyzed by next generation sequencing. FIG. 82B shows overall indel analysis between untargeted and RITDM edited HEK293 cells.

FIGS. 83A and 83B illustrates a design approach for using dCAS9-LR to target a genomic locus. FIG. 83A illustrates architectural structure of dCAS-LR as a DLR molecule. FIG. 83B illustrates dCAS-LR targeting genomic sites with a sequence-specific guide RNA.

FIG. 84 depicts data demonstrating a successful T→C genetic conversion at codon 112 of human ApoE gene by ddPCR analysis. Single nucleotide T-to-C conversions were detected by ddPCR. Left to right: H2O as no DNA control, dCAS-LR gRNA with POP98, dCAS-LR with control gRNA, dCAS9 with gRNA 1 control.

FIGS. 85A, 85B, and 86C depicts data demonstrating successful gene editing using dCAS-RITDM with two different guide RNAs at codon 112 site of human ApoE gene, measured by next generation sequencing. FIG. 85A shows SNP frequencies in untargeted HEK293 cells.

FIG. 85B shows SNP frequencies in dCAS-RITDM targeted HEK293 cells with POP98 guide RNA, with a 31.4% T→C genetic conversion frequencies at the codon 112 site. FIG. 85C shows SNP frequencies in dCAS-RITDM targeted HEK293 cells with a control ApoE a control ApoE guide RNA guide RNA, with a 10.2% T→C genetic conversion frequencies at this codon 112 site.

FIGS. 86A, 86B, and 86C shows exemplary insertion and deletion (“indel”) analysis by next generation sequencing of untargeted and dCAS-RITDM edited cells at codon 112 site of human ApoE gene in HEK293 cells. FIG. 86A shows an indel analysis at each position of a targeting region of untargeted HEK293 cells. FIG. 86B shows an indel analysis at each position of targeting of dCAS-RITDM targeted HEK293 cells with POP98 guide RNA. FIG. 86C shows an indel analysis at each position of targeting of dCAS-RITDM targeted HEK293 cells with a control guide RNA.

FIG. 87 shows overall editing frequencies and indel frequencies between untargeted and dCAS-RITDM edited HEK293 cells.

FIG. 88 is an illustration of gene expression in a normal condition.

FIG. 89 is an illustration of a mechanism of interaction between a DLR molecule and an RNA polymerase complex. In this model transcription is interrupted.

FIG. 90 is an illustration of exemplary DLR molecules used for programmed gene regulation.

FIGS. 91A and 91B show an exemplary targeting and conversion strategy demonstrated that validated DLR molecules can be used to preselect binding sites that can subsequently be used for gene regulation. FIG. 91A shows KRAS gene structure, and DNA sequences of this target, and gene conversion sequences. FIG. 91B shows ddPCR detection of GCC→TGAGAATCCG (SEQ ID NO.: 241) conversion by DLR, DLRR, and DLRRR molecules in HEK293 cells.

FIGS. 92A and 92B show RT-PCR results after programmed gene regulation. FIG. 92A shows RT-PCR strategy and FIG. 92B shows electrophoresis image of from RT-PCR reactions.

FIG. 93 shows that DLR molecules can efficiently suppress KRAS gene expression.

### DEFINITIONS

The scope of the present disclosure is defined by the claims appended hereto and is not limited by certain embodiments described herein. Those skilled in the art, reading the present specification, will be aware of various modifications that may be equivalent to such described embodiments, or otherwise within the scope of the claims. In general, terms used herein are in accordance with their understood meaning in the art, unless clearly indicated otherwise. In some instances, explicit definitions of certain terms are provided herein; meanings of these and other terms in particular instances throughout this specification will be clear to those skilled in the art from context.

As used herein, the term “adjacent” within a polynucleotide context, e.g., within a sequence context (e.g., genomic sequence, mRNA sequence, etc.), refers to adjacency of two things (e.g., components, molecules, etc.) in a linear polynucleotide (e.g., DNA) sequence and/or within a 3D chromosomal architecture of a folded genome. In some embodiments, at least one molecule as described herein comes into sufficiently close molecular proximity to, e.g., a polynucleotide, such as to be adjacent. In some such embodiments, such adjacency influences recombination events at a target site. In some embodiments, such adjacency influences gene activity (e.g. transcription) at or near a target site.

As used herein, the term “amino acid” refers to any compound and/or substance that can be incorporated into a polypeptide chain, e.g., through formation of one or more peptide bonds. In some embodiments, an amino acid has a general structure, e.g., H₂N—C(H)(R)—COOH. In some embodiments, an amino acid is a naturally-occurring amino acid. In some embodiments, an amino acid is a non-natural amino acid; in some embodiments, an amino acid is a D-amino acid; in some embodiments, an amino acid is an L-amino acid. “Standard amino acid” refers to any of the twenty standard L-amino acids commonly found in naturally occurring peptides.

“Nonstandard amino acid” refers to any amino acid, other than standard amino acids, regardless of whether it is prepared synthetically or obtained from a natural source. In some embodiments, an amino acid, including a carboxy- and/or amino-terminal amino acid in a polypeptide, can contain a structural modification as compared with general structure as shown above. For example, in some embodiments, an amino acid may be modified by methylation, amidation, acetylation, pegylation, glycosylation, phosphorylation, and/or substitution (e.g., of an amino group, a carboxylic acid group, one or more protons, and/or a hydroxyl group) as compared with a general structure. In some embodiments, such modification may, for example, alter circulating half-life of a polypeptide containing a modified amino acid as compared with one containing an otherwise identical unmodified amino acid. In some embodiments, such modification does not significantly alter a relevant activity of a polypeptide containing a modified amino acid, as compared with one containing an otherwise identical unmodified amino acid.

As used herein, the term “binding site” refers to a nucleic acid sequence within a nucleic acid molecule that is intended to be bound by an element (e.g., a D element, an R element) in a sequence-specific manner. In some embodiments, a D element (or portion thereof) and/or a sequence-specific R element (or part thereof) binds to a binding site. In some embodiments, a binding site is a site at which an element of an agent, e.g., a modification agent, e.g., a blocking agent, e.g., a DLR molecule, binds. In some embodiments, a binding site is intended to be sequence-specific, but does not have to have 100% complementarity with an agent that binds to a binding site. For example, overall binding at a binding site is sequence-specific, which means that there is substantial sequence specificity of a given element for a binding site. For instance, for a given element to bind at a binding site, in some embodiments, there may be at least 15 nucleotides that are sequence-specific although the 15 nucleotides do not necessarily need to be contiguous with one another to confer specificity.

As used herein the term “associated” refers to a relationship of two events or entities with one another as related to presence, level, degree, type and/or form. For example, a particular entity (e.g., polypeptide, genetic signature, metabolite, microbe, etc.) is considered to be associated with a particular disease, disorder, or condition, if its presence, level and/or form correlates with incidence of, susceptibility to, severity of, stage of, etc. the disease, disorder, or condition (e.g., across a relevant population). In some embodiments, two or more entities are physically “associated” with one another if they interact, directly or indirectly, so that they are and/or remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof. For example, in some embodiments, a target sequence is associated with a gene if modification, in some way, of that target sequence impacts a particular gene. In some embodiments, a protein such as an RNA polymerase is associated with a transcript when it is actively transcribing mRNA from a polynucleotide. In some such embodiments, a disruption in the association causes a dissociation of the RNA polymerase from the transcript and subsequent degradation of any partially transcribed mRNA. In some embodiments, a polymeric modification agent (e.g., a DLR molecule) is associated with one or more of a binding site, landing site, target site, target cell, target sequence, and/or target. In some embodiments, two events or entities may become dissociated from one another when their associated is disrupted or terminated.

As used herein the term “D element” refers to a sequence-specific polynucleotide (e.g., DNA) binding element. In some embodiments, a “D element” can be or comprise a naturally occurring sequence (e.g., represented by a polynucleotide) or a characteristic portion thereof, or a complement of a naturally occurring sequence or a characteristic portion thereof. In some embodiments, a D element can be or comprise one or more engineered (i.e., synthetic) nucleotides or characteristic portion(s) thereof. In some such embodiments, an engineered sequence (e.g., a sequence substantially composed of synthetic or engineered nucleotides) is analogous or corresponds to a naturally occurring sequence; however, any given engineered sequence is “produced by the hand of man.” In some embodiments D elements can include one or more of Zinc Finger proteins or domains, TALE-proteins or domains, Helix-loop-helix proteins or domains, Helix-turn-helix proteins or domains, Cas-proteins or domains (e.g., Cas9, dCas9, etc.), Leucine Zipper proteins or domains, beta-scaffold proteins or domains, Homeo-domain proteins or domains, High-mobility group box proteins or domains or characteristic portions thereof or combinations and/or parts thereof. Without being bound by any particular theory the present disclosure considers that, in some embodiments, a dissociation constant of 10E-6 or lower may confer sufficient binding strength for a given D element to bind and/or stay bound to a particular sequence.

As used herein, the term “DLR molecule” is or comprises a polymeric molecule, which molecule comprises at least one D element, an optional L element, and at least one R element, capable of binding a nucleic acid molecule. In some embodiments, a DLR molecule is arranged in the order D-L-R. In some embodiments, one or more of the D, L, and/or R elements are in an order different from D-L-R. In some embodiments, where more than one unit of any particular element is present, one of skill in the art will understand that a numeral may be used to indicate a number of a particular element, e.g., DL2R2 or DL₂R₂ or D(LR)₂, indicates a D element with two L elements bound to the D and two R elements, wherein the R elements may each be bound to the same or different L element. In some embodiments, an arrangement may also be shown as R-L-D-L-R, which would indicate that a single D element has two separate L elements bound to it, each of which has an R element bound to the L element. In some embodiments, a single D element may have more than one L element and more than one R element bound at a given time. In some embodiments, a single L element may have two R elements bound at the same time. In some embodiments, an R element may have, at either end, a sequence that functions as a linker. For example, in some embodiments, a given R element may have a sequence at an N or C-terminus a sequence that functions as a linker such that a polymeric agent (e.g., DLR molecule) is represented as DLRn, where n may be, e.g., an L element. In some embodiments, a DLR molecule has an overall dissociation constant in the same order as the lowest dissociation constant of any given component of the molecule (e.g., of a D unit, e.g., of an R unit, etc.) For example, in some embodiments, a D element and an R element of a given DLR molecule may have dissociation constants of 10E-6 or less and 10E-3 or less, respectively and, in such embodiments, a dissociation constant of a DLR molecule would be consistent with the lowest dissociation constant of a component of the molecule.

As used herein, the term “gene conversion” refers to a change in a sequence of a polynucleotide. In some embodiments, a change may be one or more of a substitution, deletion or addition of a nucleotide. In some such embodiments, a gene conversion is used to change one or more point mutations that exist in a particular gene via, e.g., a sequence modification polynucleotide. In some embodiments, a gene conversion results in a genomic genotype change that corresponds to a phenotypic change. For example, in some embodiments, a gene conversion changes a genotype from a pathogenic genotype to a functional (i.e., less pathogenic or non-pathogenic) phenotype. In some embodiments, no conversion occurs (either because no conversion has been attempted or because in a situation where one or more conversions are occurring, a particular polynucleotide is not modified). In some such embodiments, a polynucleotide and/or a cell comprising it may be referred to as “unconverted.”

As used herein, the term “genetic modification” refers to a process of gene conversion in which genetic material (e.g., a polynucleotide such as, e.g., DNA, RNA, etc.) has a difference in its sequence (e.g., genomic sequence, transcript sequence, etc.) as compared to an initial sequence (e.g., before a modification, or in a daughter cell as compared to a parent cell, etc.) at a targeted locus and/or loci. In some embodiments, a genetic modification occurs in a cell (e.g., a daughter cell). In some embodiments, a genetic modification is made using one or more technologies (e.g., systems, e.g., a RITDM system) as described herein. In some embodiments, a genetic modification may be at least one of a substitution, deletion, addition or change to molecular structure of a given nucleotide at a given target site or sites. In some embodiments, a genetic modification results in a change in a polynucleotide but no change in a corresponding polypeptide. In some embodiments, a genetic modification results in a change in a polynucleotide and a change in a corresponding polypeptide (i.e., a change in an amino acid corresponding to a triplet nucleotide). In some embodiments, where no genetic modification occurs, genetic material and/or a cell comprising such genetic material may be referred to as “unconverted.” In some embodiments, a change in activity occurs in an absence of a genetic modification. For example, in some embodiments, a polymeric modification agent may be used in absence of a sequence modification polynucleotide. In some such embodiments, in absence of a genetic modification, a change in gene regulation may still occur. For example, as described herein, in some embodiments, a polymeric modification agent, e.g., a DLR molecule, may half or reduce transcription of or at a particular target (e.g., through binding) without making a genetic modification to the nucleic acid sequence of the target.

As used herein, the term “gene regulation” refers to a process comprising a change in gene expression, including via changing transcription and/or translation of a target, target sequence and/or target site. In some embodiments, gene regulation may or may not comprise genetic modification. In some embodiments, gene regulation is or comprises downregulation (e.g., silencing, suppression, repression). For example, in some embodiments, gene regulation is accomplished by interfering with one or more components of gene transcription. That is, in some embodiments, gene regulation occurs when a polymeric modification agent, e.g., a DLR molecule, binds to a particular location on a polynucleotide that is being transcribed. In some such embodiments, the association between the polynucleotide being transcribed and the RNA polymerase is disrupted, thus disrupting and reducing a level of transcription of a target gene as supported by reduction in a level of mRNA of the target. Therefore, in some embodiments, gene regulation is or comprises gene downregulation. In some embodiments, gene regulation is or comprises gene upregulation (e.g., enhancement, increased transcription, etc.). In some such embodiments, such regulation (i.e., upregulation) of a target gene may be achieved by, for example, using a polymeric modification agent to downregulate another gene that silences or represses or otherwise inhibits expression, thus by downregulating the inhibitory component, upregulation occurs.

As used herein, the term “genomic engineering” refers to a process that involves deliberate modification of one or more characteristics of genetic material or one or more mechanisms for expressing genetic material. For example, in some embodiments, gene editing is accomplished using genomic engineering. In some embodiments, gene regulation is accomplished using genomic engineering. In some such embodiments, such gene regulation is or comprises up or downregulated of expression of one or more genes by modification of processing activities (e.g., transcription). In some embodiments, genomic engineering occurs in vivo, within the genome of one or more cells of an organism. In some embodiments, genomic engineering occurs in vitro or ex vivo, within a gene or polynucleotide that may or may not be encompassed within a genome, but is encompassed within a cell (e.g., natural cell, engineered cell, artificial cell, etc.). As used herein, the term “identity” refers to the overall relatedness between polymeric molecules, e.g., between nucleic acid molecules (e.g., DNA molecules and/or RNA molecules) and/or between polypeptide molecules. In some embodiments, polymeric molecules are considered to be “substantially identical” to one another if their sequences are at least 75%, 80%, 85%, 90%, 95%, 96%, 97%, 98%, 99% or more identical. Calculation of the percent identity of two nucleic acid or polypeptide sequences, for example, can be performed by aligning the two sequences for optimal comparison purposes (e.g., gaps can be introduced in one or both of a first and a second sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, at least 96%, at least 97%, at least 98%, at least 99% or substantially 100% of the length of a reference sequence. The nucleotides at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. As will be understood to those of skill in the art, comparison of sequences and determination of percent identity between two sequences can be accomplished using a mathematical algorithm.

As used herein, the term “landing site” refers to a nucleic acid sequence to which a sequence-specific element (e.g., a D-element, an R-element, etc.) is targeted (e.g., to bind to it). In some embodiments a landing site may overlap with a target site (e.g., have nucleotides that are part of both a landing site and a target site). In some embodiments, a landing site may comprise a target site or a portion thereof. In some embodiments, a landing site may be in relatively close proximity (e.g., adjacent) to a target site. In some embodiments, a landing site may be a distance away from a target site. In some such embodiments, where a landing site is a distance away from a target site, it is still considered a landing site as long as cellular modification processes enable modification of, at, or associated with a target site (e.g., genetic modification, gene regulation, etc.).

As used herein, the term “L element” or “linker” refers to an element that links at least one D element to at least one R element. An L element can be an existing, naturally occurring, engineered, designed and/or selected molecule. In some embodiments, an L element is an optional component in a composition and/or molecule comprising a D and/or an R element. In some embodiments, an L element has no function other than to link one or more D elements to one or more R elements. In some embodiments, an L element does have a function beyond simply linking (e.g., positioning one or both of a D element and/or an R element to support a particular application or modification, serving as a site for action of an enhancing agent). In some embodiments, a primary function of an L element is to link a D element with an R element. In some embodiments, in addition to serving a linker function, an L element may have additional features or functions. For example, in some embodiments, an L element may facilitate or participate in orientation of a given DLR molecule relative to one or more molecules (e.g., DNA, RNA, etc.) to which it is bound. In some embodiments, such additional features or functions may serve to enhance overall impact or functionality of a given DLR molecule. In some embodiments, an L element may impact binding strength of a DLR molecule. For example, in some embodiments, an L element may increase binding strength of a given DLR molecule. For instance, by way of non-limiting example, if an L element is or comprises one or more basic amino acid residues it may serve to interact more strongly with a negatively charged molecule (e.g., a DNA backbone). In some embodiments, an L element may contribute to sequence specificity or sequence specific interactions of a given DLR molecule with a given target. In accordance with various embodiments, an L element may be of any application-appropriate length and composition. For example, in some embodiments, an L element will be long enough to allow that both elements “D” and “R” are simultaneously bound to a DNA molecule. In some embodiments, an L element is between 1 and 100 amino acids (e.g., 1-50, 2-20, 2-10, 2-5, 2-4 amino acids or longer). In some embodiments, an L element is flexible. In some embodiments, an L element is semi-flexible. In some embodiments, an L element is rigid.

As used herein, the term “nuclease” is an enzyme capable of cleaving one or more bonds in a polynucleotide, typically by hydrolyzing one or more phosphodiester bonds between individual nucleotides. In some embodiments, a nuclease is a protein, e.g., an enzyme that can bind a polynucleotide and cleave a phosphodiester bond connecting nucleotide residues within the polynucleotide. In some embodiments, a nuclease is site-specific. In some such embodiments, such a nuclease binds and/or cleaves a specific phosphodiester bond within a specific polynucleotide of a particular sequence, which is also referred to herein as a “target site.” In some embodiments, a nuclease causes a break in a polynucleotide. In some such embodiments, such breaks can be single-stranded or double-stranded in that a single-stranded break is a break that occurs in a single-polynucleotide strand (in a single or double-stranded molecule) and a double-stranded break is one that occurs between at least two nucleotides on one strand and the complementary nucleotides on an opposite strand of a double-stranded molecule. Nucleases can be naturally existing macromolecules or parts thereof; they can be modified versions thereof or can be designed or engineered. In some embodiments, nucleases have a 3-dimensional fold in which certain amino acids form a catalytic core that can perform catalytic hydrolysis. In some embodiments, nuclease or nuclease-like domains can be incorporated into larger macromolecules.

As used herein, the term “nucleic acid” refers to any element that is or may be incorporated into a polynucleotide chain. In some embodiments, a nucleic acid may be incorporated into a polynucleotide chain via phosphodiester linkage. In some embodiments, nucleic acids are polymers of deoxyribonucleotides or ribonucleotides. In some such embodiments, deoxyribonucleotides or ribonucleotides may be synthetic oligonucleotides. As will be clear from context, in some embodiments, “nucleic acid” refers to an individual nucleic acid residue (e.g., a nucleotide and/or nucleoside); in some embodiments, “nucleic acid” refers to a polynucleotide comprising individual nucleic acid residues. In some embodiments, a polymer or deoxyribonucleotides and/or ribonucleotides can be single-stranded or double-stranded and in in linear or circular form. Polynucleotides comprised of nucleic acids can also contain synthetic or chemically modified analogues of ribonucleotides, in which a sugar, phosphate and/or base units are modified. In some embodiments, a “nucleic acid” is or comprises RNA; in some embodiments, the RNA is or comprises mRNA. In some embodiments, a “nucleic acid” is or comprises DNA. In some embodiments, a nucleic acid is, comprises, or consists of one or more natural nucleic acid residues. In some embodiments, a nucleic acid is, comprises, or consists of one or more nucleic acid analogs. In some embodiments, a nucleic acid analog differs from a nucleic acid in that it does not utilize a phosphodiester backbone. Alternatively or additionally, in some embodiments, a nucleic acid has one or more phosphorothioate and/or 5′-N-phosphoramidite linkages rather than phosphodiester bonds. In some embodiments, a nucleic acid is, comprises, or consists of one or more natural nucleosides (e.g., adenosine, thymidine, guanosine, cytidine, uridine, deoxyadenosine, deoxythymidine, deoxyguanosine, and deoxycytidine). In some embodiments, a nucleic acid is, comprises, or consists of one or more nucleoside analogs. In some embodiments, a nucleic acid comprises one or more modified sugars as compared with those in natural nucleic acids. In some embodiments, a polynucleotide is comprised of at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues. In some embodiments, a polynucleotide is or comprises a partly or wholly single stranded molecule; in some embodiments, polynucleotide is or comprises a partly or wholly double stranded.

As used herein, the term “polymeric modification agent” refers to an agent that modifies, in some way, a polynucleotide sequence and/or expression activity. For example, in some embodiments, a polymeric modification agent binds to a binding site and, in conjunction with a sequence modification polynucleotide, modifies a gene sequence associated with a target. In some embodiments, a polymeric modification agent in absence of a sequence modification polynucleotide modifies gene activity. For example, in some embodiments, a polymeric modification agent disrupts association of an RNA polymerase with a transcript, decreasing gene transcription and mRNA production. In some embodiments, as will be understood by context, a polymeric modification agent may be or comprise one or more of blocking agent such as a gene modification agent (e.g., a sequence modification agent) and/or a gene regulation agent (e.g., a transcription modification agent), an enhancing agent, an inhibiting agent, etc.

As used herein, the term “polynucleotide” refers to any polymeric chain of nucleic acids. In some embodiments, a polynucleotide is or comprises RNA. In some such embodiments, the RNA is or comprises mRNA. In some embodiments, a polynucleotide is or comprises DNA. In some embodiments, a polynucleotide is, comprises, or consists of one or more natural nucleic acid residues. In some embodiments, a polynucleotide is, comprises, or consists of one or more nucleic acid analogs. In some embodiments, a polynucleotide analog differs from a nucleic acid in that it does not utilize a phosphodiester backbone. Alternatively or additionally, in some embodiments, a polynucleotide has one or more phosphorothioate and/or 5′-N-phosphoramidite linkages rather than phosphodiester bonds. In some embodiments, a polynucleotide is, comprises, or consists of one or more natural nucleosides (e.g., adenosine, thymidine, guanosine, cytidine, uridine, deoxyadenosine, deoxythymidine, deoxyguanosine, and deoxycytidine). In some embodiments, a polynucleotide is, comprises, or consists of one or more nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, 2-thiocytidine, methylated bases, intercalated bases, and combinations thereof). In some embodiments, a polynucleotide comprises one or more modified sugars (e.g., 2′-fluororibose, ribose, 2′-deoxyribose, arabinose, and hexose) as compared with those in natural nucleic acids. In some embodiments, a polynucleotide has a nucleotide sequence that encodes a functional gene product such as an RNA or protein. In some embodiments, a polynucleotide is prepared by one or more of isolation from a natural source, enzymatic synthesis by polymerization based on a complementary template (in vivo or in vitro), reproduction in a recombinant cell or system, and chemical synthesis. In some embodiments, a polynucleotide is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long. In some embodiments, a polynucleotide is partly or wholly single stranded. In some embodiments, a polynucleotide is partly or wholly double stranded. In some embodiments, a polynucleotide has a nucleotide sequence comprising at least one element that encodes, or is the complement of a sequence that encodes, a polypeptide. In some embodiments, a polynucleotide has enzymatic activity.

As used herein, the term “polypeptide” refers to any polymeric chain of residues (e.g., amino acids) that are typically linked by peptide bonds. In some embodiments, a polypeptide has an amino acid sequence that occurs in nature. In some embodiments, a polypeptide has an amino acid sequence that does not occur in nature. In some embodiments, a polypeptide has an amino acid sequence that is engineered in that it is designed and/or produced through action of the hand of man. In some embodiments, a polypeptide may comprise or consist of natural amino acids, non-natural amino acids, or both. In some embodiments, a polypeptide may include one or more pendant groups or other modifications, e.g., modifying or attached to one or more amino acid side chains, at a polypeptide's N-terminus, at a polypeptide's C-terminus, or any combination thereof. In some embodiments, such pendant groups or modifications may be acetylation, amidation, lipidation, methylation, pegylation, etc., including combinations thereof. In some embodiments, polypeptides may contain L-amino acids, D-amino acids, or both and may contain any of a variety of amino acid modifications or analogs known in the art. In some embodiments, useful modifications may be or include, e.g., terminal acetylation, amidation, methylation, etc. In some embodiments, a protein may comprise natural amino acids, non-natural amino acids, synthetic amino acids, and combinations thereof. The term “peptide” is generally used to refer to a polypeptide having a length of less than about 100 amino acids, less than about 50 amino acids, less than 20 amino acids, or less than 10 amino acids. In some embodiments, a protein is antibodies, antibody fragments, biologically active portions thereof, and/or characteristic portions thereof.

As used herein the term “R element” refers to a polynucleotide (e.g., DNA)-binding molecule (e.g., a macromolecule, e.g., an oligonucleotide, etc.) that binds to a polynucleotide that is different, e.g., opposite, a strand to which a sequence-specific D element binds. In some embodiments, an R-element binds to an opposite DNA strand than to where a D element is bound (i.e., lagging strand). In some embodiments, an R element can bind in a sequence specific manner or it can bind in a non-sequence specific (e.g., positional, etc.) manner. In some such embodiments, an R element may bind to DNA, RNA, mRNA, etc. In some embodiments, an R element is present within the same molecule as a given D element, but the D element and R element may be bound to two separate molecules, e.g., two separate DNA molecules; for example, a D element may be bound to a leading strand at or near a replication fork and an R element may be bound to a lagging strand at or near a replication fork, but on a separate DNA molecule than where the D element of a given DLR molecule is bound. In some embodiments, an R element binds to a polynucleotide with sufficient affinity (e.g., a dissociation constant of at least 10E-3 or less) to slow or stall polynucleotide processing (e.g., DNA replication, e.g., transcription, e.g., translation). In some embodiments, an R element of a given DLR molecule binds less strongly than a D element of the same molecule. In some embodiments, an R and D element of a given DLR molecule bind with similar affinities. In some embodiments, an R element binds in a sequence-specific manner; in some such embodiments, an R element and a D element of a given DLR molecule may bind with similar affinities (e.g., dissociation constant of 10E-6 or less, etc.). In some embodiments sequence specific interaction can be achieved through similar means as described and provided for and by a D element, however, in any given DLR molecule binding of an R element is different from that of a D element in that can be different from a D element (e.g., D element: engineered zinc finger protein combined with an R-element that comprises a CAS-protein). In some embodiments non-sequence specific interaction of sufficient affinity can be achieved through structures that can interact through various interactions such as, e.g., phosphate backbone interactions and/or hydrophobic/Van der Waals interactions with a major and/or minor groove of a DNA molecule. In some embodiments an R element can combine elements that result in non-sequence specific and -sequence-specific interactions. In some such embodiments, non-sequence specific and sequence specific interactions occur sequentially. In some embodiments, non-sequence specific and sequence specific interactions occur substantially simultaneously. In some embodiments, an R element can be or comprise a naturally occurring sequence or characteristic portion thereof. In some embodiments, an R element can.be or comprise an engineered sequence or characteristic portion thereof. In some such embodiments, an engineered sequence is analogous or corresponds to a naturally occurring sequence; however, any given engineered sequence is “produced by the hand of man.” In some embodiments an R-element binds to one or more regions which may be or comprise a Zinc Finger protein or domain, TALE protein or domain, Helix-loop-helix protein or domain, Helix-turn-helix protein or domain, CAS protein or domains Leucine Zipper protein or domain, beta-scaffold protein or domain, Homeo-domain protein or domain, High-mobility group box protein or domain or a combination thereof. In some embodiments, R elements may be engineered or designed such that binding interactions between R elements and a polynucleotide are different from naturally occurring binding interactions (e.g., an R element may bind to an engineered lagging DNA strand, etc.). In some embodiments R elements have little to no sequence specificity; for example, in some embodiments, R elements can be engineered, designed or selected to have little or no sequence specificity (e.g., no nucleotide and/or amino acid specificity). For instance, in some embodiments R elements can be engineered or designed to have a three-dimensional structure that can bind a given polynucleotide molecule (e.g., a DNA molecule) in a non-sequence specific manner. In some such embodiments such a structure can be based on a structural feature (e.g., fold) that may be present in a naturally occurring protein (e.g., polymerases, DNases, etc.) that interacts with a given polynucleotide (e.g., DNA, mRNA, etc.). In some embodiments specific amino acids are changed (as compared to those in a naturally occurring protein), for example an amino acid that may be involved in an active site may be changed such that the catalytic function is reduced and/or abolished. In some embodiments R elements are designed that are hybrids of naturally occurring folds and/or designed folds. In some embodiments, non-sequence specific binding by R elements can occur via one or more types of interactions known to those of skill in the art; for example, interactions of an R-element with a sugar phosphate backbone of a molecule to which it binds, hydrophobic interactions involving a minor or major groove of a DNA molecule to which an R-element binds or interacts, etc. As will be appreciated by one of skill in the art, such interactions are generally not explicitly sequence-specific, per se.

As used herein the term “Replication Interrupted Template driven DNA Modification” or “Recombination Induced Template Driven DNA Modification” (RITDM) refers to an editing system that modifies (e.g., changes via deletion, addition, substitution, etc.) a given polynucleotide (e.g., DNA, RNA, mRNA, etc.) in a cell without doing so by causing a single and/or double-stranded break in a given polynucleotide (e.g., DNA, RNA, etc.) being modified. As will be appreciated by those of skill in the art a RITDM system may comprise polynucleotide (e.g., DNA) modification such as deletion, addition, substitution, etc. of one or more nucleotides using, for example, replication interruption (e.g., of a DNA replication process) and/or recombination (e.g., at a target site) methods by combining a polymeric modification agent (e.g., a DLR molecule) and, in some embodiments, a sequence modification polynucleotide and/or additional agent (e.g., guide RNA). In some embodiments a RITDM system comprises (i) a blocking agent (e.g., a DLR molecule) and (ii) a sequence modification polynucleotide. In some such embodiments, the blocking agent binds to, e.g., double-stranded DNA. In some embodiments, strength of binding of, e.g., a blocking agent, e.g., a DLR molecule, is sufficient to slow or stall a replication fork during DNA replication. In some embodiments a DLR molecule, in combination with a sequence modification polynucleotide, may result in a genetic modification.

As used herein, the term “sample” refers to a portion or aliquot of a material obtained or derived from a source of interest, as described herein. In some embodiments, a source of interest is a biological or environmental source. In some embodiments, a source of interest may be or comprise a cell or an organism, such as a microbe, a plant, or an animal (e.g., a human). In some embodiments, an organism is a pathogen (e.g., an infectious pathogen, e.g., a bacterial pathogen, a viral pathogen, a parasitic pathogen, etc.). In some embodiments, a source of interest is or comprises biological tissue or fluid. In some embodiments, a biological tissue or fluid may be or comprise amniotic fluid, aqueous humor, ascites, bile, bone marrow, blood, breast milk, cerebrospinal fluid, cerumen, chyle, chime, ejaculate, endolymph, exudate, feces, gastric acid, gastric juice, lymph, mucus, pericardial fluid, perilymph, peritoneal fluid, pleural fluid, pus, rheum, saliva, sebum, semen, serum, smegma, sputum, synovial fluid, sweat, tears, urine, vaginal secretions, vitreous humour, vomit, and/or combinations or component(s) thereof. In some embodiments, a biological fluid may be or comprise an intracellular fluid, an extracellular fluid, an intravascular fluid (blood plasma), an interstitial fluid, a lymphatic fluid, and/or a transcellular fluid. In some embodiments, a biological fluid may be or comprise a plant exudate. In some embodiments, a biological tissue or sample may be obtained, for example, by aspirate, biopsy (e.g., fine needle or tissue biopsy), swab (e.g., oral, nasal, skin, or vaginal swab), scraping, surgery, washing or lavage (e.g., brocheoalveolar, ductal, nasal, ocular, oral, uterine, vaginal, or other washing or lavage). In some embodiments, a biological sample is or comprises cells obtained from an individual. In some embodiments, a sample is a primary sample in that it is obtained directly from a source of interest by any appropriate means. In some embodiments, as will be clear from context, a sample refers to a preparation that is obtained by processing (e.g., by removing one or more components of and/or by adding one or more agents to) a primary sample. For example, processing a sample for testing to extract genetic material for genetic analyses such as by, e.g., applying one or more solutions, separating components using a semi-permeable membrane, etc. Such a “processed sample” may comprise, for example nucleic acids or proteins extracted from a sample or obtained by subjecting a primary sample to one or more techniques such as amplification or reverse transcription of nucleic acid, isolation and/or purification of certain components, etc. In some embodiments, a sample is used to design one or more DLR molecules and/or sequence modification polynucleotides as provided herein.

As used herein, the term “sequence modification polynucleotide” refers to a polynucleotide that has substantial homology with a target sequence (e.g., a genomic sequence, a transcript, etc.), but is not identical to that target sequence. In some embodiments a sequence modification polynucleotide may have properties equivalent to a wild-type polynucleotide, but may be chemically modified and/or use synthetic or chemically modified building blocks. In some embodiments, a sequence modification polynucleotide is used in conjunction with a blocking agent (e.g., a DLR molecule) in order to achieve sequence modification at a target site. For example, in some embodiments, a sequence modification polynucleotide is a donor template in that such a polynucleotide provides one or more nucleic acids for incorporation into a given sequence (e.g., a genomic sequence, a transcript, etc.). In some embodiments, a sequence modification polynucleotide is a correction template in that it is used in a cellular process (e.g., a replication process) as a “guide” of sorts by cellular machinery in order to make a change (e.g., a substitution, deletion, addition) to a given polynucleotide (e.g., DNA, RNA, etc.), In some embodiments, a sequence modification polynucleotide may contain a “wild-type” nucleic acid sequence that is almost entirely identical or homologous to a variant sequence except for one or two nucleotides (i.e., point mutations, substitutions, etc.) that is/are regarded as changed relative to the wild type sequence (i.e., a variant sequence). In some embodiments, a sequence modification polypeptide such as a donor template may differ by only a single nucleotide relative to a wild-type sequence. In some embodiments, a sequence modification polypeptide may have two or more nucleotide differences relative to a wild-type sequences. In some such embodiments, such a polypeptide may have multiple nucleotides differences in a target sequence as compared to a wild-type sequence. A sequence modification polynucleotide may be at least about 10 nucleotides to at least about 20 kb in length. In some embodiments, an sequence modification polynucleotide is or comprises a template which itself is not necessarily incorporated into, e.g., a replicating nucleic acid strand, but the sequence of the sequence modification polynucleotide is reflected in a replicated nucleic acid strand (e.g., a nucleic acid strand is edited after contact with a sequence modification polynucleotide even if the physical sequence modification polynucleotide itself is not incorporated into the strand). In some embodiments, a sequence modification polynucleotide has or comprises a sequence that is at least 50%, 60%, 70%, 75%, 80%, 85%, 90%, 95%, 96%, 97%, 98%, 99%, 99.1%, 99.2%, 99.3%, 99.4%, 99.5%, 99.6%, 99.7%, 99.85, or 99.9% or greater identical to a target sequence and/or target site. In some embodiments, a sequence modification polynucleotide has or comprises a sequence that is at most approximately 99.9%, 99.8%, 99.7%, 99.6%, 99.5%, 99.4%, 99.3%, 99.2%, 99.1%, 99%, 98%, 97%, 96%, 95%, 90%, 85%, 80%, 75%, 70%, 65%, 60%, 55%, 50%, 45%, 40%, 35%, 30%, 25%, 20%, 15%, 10%, 5%, 1%, or 0% identical to a target site or sequence as provided herein. In some embodiments, identity is over a particular size or length of target size or sequence. In some embodiments, identity does not refer to a contiguous sequence. In some embodiments, identity does refer to a contiguous sequence. In some embodiments, such as when a polymeric blocking agent is used to for gene regulation such as to block, inhibit, reduce or otherwise disrupt transcription activity, no sequence modification polynucleotide is used.

As used herein, the term “sequence-specific binding” refers to an event that occurs when a macromolecule (e.g., a protein, peptide, polypeptide, nucleotide comprising protein) interacts with a polynucleotide (e.g., DNA, RNA, mRNA, etc.), and at least a sub-set (e.g., 2, 3, 4, 5, 6, 7, 8, 9, 10 or more) of contacts between a macromolecule and a polypeptide is sequence-specific in that expected portions of each molecule interact with one another (e.g., Arginine interacting with Guanidine; other exemplary interactions will be known to those of skill in the art and can be found, for instance, in various descriptions throughout the literature describing DNA recognition codes for zinc fingers). As is understood by those of skill in the art, not every interaction between every portion of each molecule needs to be sequence specific; however the overall interaction between two molecules interacts, generally, in a manner that is sequence-specific. In some embodiments an overall dissociation constant for interaction will be 10E-6 or less. As will be appreciated by those of skill in the art, a smaller dissociation constant indicates stronger binding. In some embodiments sequence-specific binding will entail interaction in which at least three base pairs or nucleotides are bound with sufficient affinity and selectivity, such that other sequences will be bound at levels less than 50% of a desired or targeted DNA sequence.

As used herein, the term “subject” refers to an organism. In some embodiments, a subject is an individual organism. A subject may be of any chromosomal gender and at any stage of development, including prenatal development. In some embodiments a subject is comprised of, either wholly or partially, eukaryotic cells (e.g., an insect, a fly, a nematode). In some embodiments, a subject is a vertebrate. In some embodiments, a subject is a mammal. In some embodiments, a mammal is a human, including prenatal human forms. In some embodiments, a subject is suffering from a relevant disease, disorder or condition. In some embodiments, a subject is susceptible to a disease, disorder, or condition. In some embodiments, a subject displays one or more symptoms or characteristics of a disease, disorder or condition. In some embodiments, a subject does not display any symptom or characteristic of a disease, disorder, or condition. In some embodiments, a subject is someone with one or more features characteristic of susceptibility to or risk of a disease, disorder, or condition. In some embodiments, a subject is a patient. In some embodiments, a subject is an individual to whom diagnosis and/or therapy is and/or has been and/or will be administered.

As used herein, the term “target” refers to a particular gene, region (e.g., promoter, enhancer, UTR, etc.) or other location or component in a cell that is impacted by a polymeric modification agent of the present disclosure. For example, in some embodiments, a target is a gene or genomic region and a polymeric modification agent, in conjunction with a sequence modification polynucleotide, may act to modify one or more nucleotides in a target. In some embodiments, a target is a cell complex such as a polymerase and polynucleotide; for example, an RNA polymerase and strand of DNA and/or mRNA. A target may or may not be or comprise a landing site or a binding site or a portion thereof. In some embodiments, a target is or comprises a target sequence and/or target site. A target may or may not comprise a non-methylated, partially-methylated, or wholly-methylated region.

As used herein, the term “target cell” or “targeted cell” refers to a cell that has been contacted with at least one polymeric modification agent (e.g., a DLR molecule) and, optionally, at least one sequence modification polynucleotide. In some embodiments, a target cell comprises at least one nucleic acid change at a target site as compared to the same cell prior to the application of the at least one polymeric modification agent and at least one sequence modification polynucleotide, or, in some embodiments, as compared to another targeted cell or an untargeted cell. In some embodiments, a target cell does not comprise a nucleic acid change at a target site as compared to an untargeted cell. In some embodiments, a targeted cell may have one or more nucleic acid differences as compared to an untargeted cell, but is still not an edited cell as the one or more differences may not be at or within a target site. A targeted cell may or may not be an edited cell. In some embodiments, a targeted cell is an edited cell in that its nucleic acid sequence has been successfully edited in a specific and intended way, e.g., reflecting a designed genetic change based upon a supplied sequence modification polynucleotide. In some embodiments, an edited cell has a specific nucleotide sequence in which technologies of the present disclosure are used to make one or more nucleotide modifications (e.g., substitutions, additions, deletions, etc.) relative to, for example, a control cell or a targeted cell that is not an edited cell. For example, in some embodiments, an untargeted cell or a targeted but unedited cell, does not reflect a specific sequence (i.e., is not edited) provided using a sequence modification polynucleotide. In some embodiments, a targeted, edited cell may have one or more additional changes in addition to changes introduced via a sequence modification polynucleotide (e.g., SNP). In some embodiments, a targeted but unedited cell and/or an untargeted cell may have one or more genetic changes as compared to an earlier version of a cell or a control, but does not have or comprise a particular sequence provided by a sequence modification polynucleotide. For example, in some embodiments, one or more SNPs may be detected but such SNPs may not be in a vicinity of a target site. In some embodiments, a target cell comprises a reduced level of transcription and/or mRNA of a target as compared to a cell that has not been contacted by a polymeric modification agent.

As used herein, the term “target sequence” refers to a particular sequence comprising one or more nucleic acids to be modified using technologies of the present disclosure. In some embodiments, a target sequence is or comprises one or more nucleotides. In some embodiments, a target sequence is modified by a change in its association with one or more other entities or elements. For example, in some embodiments, a target sequence is modified by a change that impacts gene regulation. For example, in some such embodiments, a target sequence is modified by dissociation of a protein (e.g., an RNA polymerase) from a transcript associated with or comprising a target sequence. That is, in some embodiments, a RNA polymerase is dissociated from a transcript that is associated, in some way, with a target sequence. In some embodiments, a target sequence is wholly naturally-occurring. In some embodiments, a target sequence is or comprises one or more synthetic nucleotides or components. In some embodiments, a target sequence is or comprises both naturally occurring or synthetic components (e.g., nucleic acid residues, etc.).

As used herein, the term “target site” refers to a location (e.g., a particular genome, chromosome, chromosomal position, etc.) of a given nucleic acid sequence within a nucleic acid molecule that comprises a target sequence, which target sequence is intended to be modified by a RITDM system or via gene regulation by one or more polymeric modification agents as described herein. For example, in some embodiments, a target site is or comprises a nucleotide that is targeted for a change (e.g., replacement via substitution, removal, addition, etc.). In some such embodiments, a target site is a sequence-specific target site. In some embodiments, a target site is a structure specific target site. In some embodiments, a target site is both sequence and target specific. In some embodiments, a target site is non-sequence and/or non-structure specific. In some embodiments, a target site compromises a sequence associated with a disease, disorder or condition. In some embodiments, a target site is or comprises a polynucleotide sequence, e.g., a DNA sequence, that comprises a point mutation associated with a disease, disorder or condition. In some such embodiments, a target site may be or comprise an error site (e.g., a site where presence of one or more nucleotides is associated with existence, development or risk of a disease, disorder, or condition). In some such embodiments, a target site is or comprises a target sequence or portion thereof that is modified by a gene regulation process. For example, in some such embodiments, a target site may be associated with a gene that is regulated by a change in a relationship with one or more other elements; for example, in some embodiments, a target site, in whole or in part, may be part of a transcript that is being transcribed by an RNA polymerase that is dissociated by a polymeric modification agent.

As used herein, the terms “treat” or “treatment” refer to any technology as provided herein that is used to partially or completely alleviate, ameliorate, relieve, inhibit, prevent, delay onset of, reduce severity of, and/or reduce incidence of one or more symptoms or features of a disease, disorder, and/or condition. In some embodiments of the present disclosure a treatment may be or comprise changing a genotype in a subject. In some embodiments, treatment may be administered to a subject who does not exhibit signs of a disease, disorder, and/or condition. In some embodiments, treatment may be administered to a subject who exhibits only early signs of the disease, disorder, and/or condition, for example for the purpose of decreasing the risk of developing pathology associated with the disease, disorder, and/or condition. In some embodiments, treatment refers to administration of a therapy (e.g., composition, pharmaceutical composition, e.g., DLR molecule and/or sequence modification agent and/or enhancing and/or inhibiting agent, etc.) that partially or completely alleviates, ameliorates, relives, inhibits, delays onset of, reduces severity of, and/or reduces incidence of one or more symptoms, features, and/or causes of a particular disease, disorder, and/or condition. In some embodiments, such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition. Thus, in some embodiments, treatment may be prophylactic; in some embodiments, treatment may be therapeutic.

### DETAILED DESCRIPTION

Gene editing and genomic engineering hold great promise. For instance, many types of editing or engineering could be useful in treating one or more diseases, disorders or conditions. Gene editing and genomic engineering offer an advantage that, in some embodiments, they can be very precise. The present disclosure recognizes that an ideal approach to gene editing would encompass features such as being (1) safe and with few to no off-target effects; (2) versatile ability to convert all types of variants (e.g., differences relative to wild-type) to a desired genotype (e.g., a wild-type genotype, a codon-optimized genotype, etc.) or behavior (e.g., expression pattern or activity); and (3) be sufficiently effective to be of practical use. None of the currently existing methods for gene editing and genomic engineering fulfills all three criteria. The present disclosure appreciates that one challenge with currently available gene editing approaches that use nucleases and/or nickases is that they necessarily generate double stranded DNA or single stranded DNA breaks, respectively; that is, the mechanism by which these approaches function is by creating single or double-stranded breaks in a given molecule. In some embodiments, the present invention recognizes that some such breaks may lead to chromosomal rearrangements, etc. In some such embodiments, such rearrangements will typically elicit DNA repair mechanisms, e.g., Non Homologous End Joining (NHEJ). In some embodiments, NHEJ can be mutagenic. The present disclosure provides innovative technologies that are designed, among other things, to overcome limitations of current technologies. For example, in some embodiments, methods of the present disclosure are designed to function without generating one or more breaks, e.g., in a polynucleotide, e.g., in a DNA molecule, etc. As will be appreciated by one of skill in the art, previous methods have attempted genomic engineering and/or gene editing without introducing DNA breaks; however, these methods have also included, for example, viruses, which can, in some embodiments, introduce foreign (e.g., viral) DNA into a eukaryotic host. Other methods use polynucleotides such as oligonucleotides to try to achieve gene conversion and/or gene correction, which, in some embodiments, can have insufficient efficacy to make their use practical (e.g., 10E-5 to 10E-6 for mammalian cells) as a sole method of genomic modification In addition, in some embodiments, use of oligonucleotides as a sole strategy for gene conversions may require positive selection (e.g., such as via antibiotic resistance markers or fluorescent markers) in order to isolated converted cells. Other methods such as, e.g., “base editors” are generally only available for making single, specific base substitutions; thus, if, for example, more than one substitution is required or, if, for example a change that is a deletion or addition of a nucleotide is needed, a base editor is not an appropriate choice.

Thus, as described herein, the present disclosure provides technologies (e.g., systems, agents, methods, etc.) related to gene/genome editing and/or genomic engineering. As will be appreciated by those of skill in the art, such technologies have a wide array of applications. In some embodiments, the present disclosure provides blocking agents.

### Replication Interrupted or Recombination Induced Template Driven DNA Modification (RITDM)-Mediated Gene Editing and Genomic Engineering

The present disclosure recognizes that, among other things, it would be advantageous to be able to achieve gene and/or genome editing or engineering without needing to introduce one or more breaks into genetic material (e.g., DNA, RNA, etc.). As provided herein, technologies of the present disclosure are based upon the discovery that gene or genome editing can be performed using a newly developed agent that can achieve gene editing or genome engineering without having to introduce one or more breaks in, e.g., a polynucleotide chain. For example, in some embodiments the present disclosure provides one or more agents to achieve such gene or genome editing. In some embodiments, an agent is a sequence-specific binding molecule that, in combination with a sequence modification polynucleotide, can be introduced into a cell to achieve genetic modification (e.g., DNA modification, RNA modification) without the administered agent creating single- or double-stranded breaks in endogenous polynucleotides (e.g., DNA, etc.).

A key aspect of the present disclosure, including the RITDM system, is that, in some embodiments, use of a RITDM system contacts a cell with a sequence-specific DNA binding molecule and a sequence modification template (e.g., donor template). For example, in some embodiments, a sequence-specific DNA binding molecule is a DLR agent as described and provided herein. In some embodiments, a DLR agent is engineered by combination of various elements providing a sequence-specific DNA binding activity at a target sequence in a genome. In some embodiments, a sequence modification polynucleotide (e.g., template, e.g., a donor template, e.g., a correction template) carries a genetic modification (e.g., a polynucleotide modification) relative to a sequence of a target site. In some such embodiments, a sequence modification polynucleotide is capable of annealing to one strand of nucleic acid (e.g., a lagging strand at a DNA replication fork, e.g., at a stalled replication fork, e.g., at a replication fork to which at least one component of an agent, e.g., a DLR agent, is bound) at a target site, e.g., in a genome. In some embodiments a polymeric modification agent, e.g., a blocking agent (e.g., a DLR agent, e.g., a DLR molecule) and a sequence modification polynucleotide (e.g., donor template, e.g., correction template) will be administered to and/or administered to a cell. In some embodiments, a polymeric modification agent, e.g., a blocking agent, and a sequence modification agent are simultaneously present in a given cell. In some embodiments, in addition to a polymeric modification agent, e.g., a blocking agent, and a sequence modification agent, an enhancing or inhibiting agent (e.g., an siRNA, etc.) may also be administered. In some embodiments, more than one polymeric modification agent, e.g., a blocking agent, sequence modification polynucleotide and/or enhancing or inhibiting agent, (e.g., siRNA) may be administered to and/or presented to a cell.

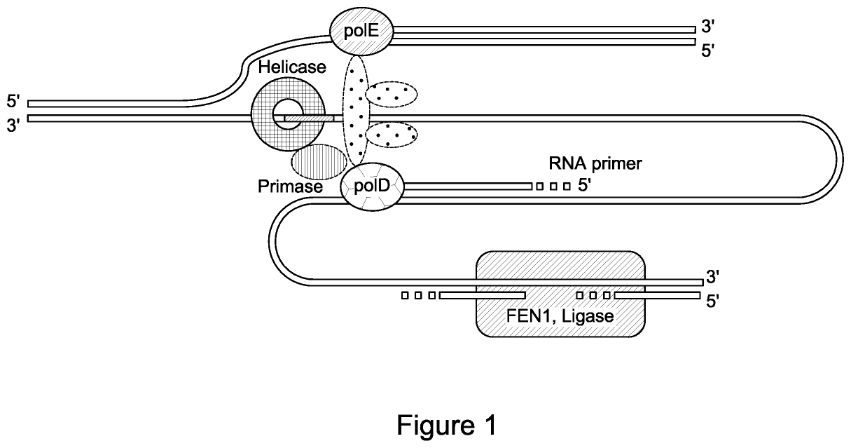

Without being bound by any particular theory, the present disclosure contemplates that temporarily slowing down or stalling DNA replication (e.g., with a blocking agent) will facilitate a sequence modification (e.g., via a sequence modification polypeptide.) For example, as will be appreciated by one of skill in the art, FIG. 1 illustrates a schematic of a DNA replication. Generally, during DNA replication, a replication complex “unwinds” a double-helical conformation of a given DNA molecule and as this unwinding occurs, both a “leading” and “lagging” single strands are present and each being replicated via replication machinery. It is generally understood that under “normal” (e.g., homeostatic) conditions, a leading strand can be replicated in a continuous process and a corresponding lagging strand has a more complex replication mechanism which, in some embodiments, involves synthesis of Okazaki fragments. The present disclosure appreciates that during the replication process, when leading and lagging strands are exposed as single strands and, in particular, the lagging strand has not yet been replicated, a wholly single stranded portion of DNA is exposed, albeit for a very short duration of time.

Accordingly, the present disclosure provides the insight that developing technologies (e.g., systems, compositions, methods) to temporarily slow or stall a polynucleotide process, (e.g., replication, e.g., transcription) expands the duration of time that a single strand (e.g., a lagging strand during DNA replication) is exposed. Thus, for example, in some embodiments, exposure of a single strand such as, e.g., a lagging DNA strand, is then available for binding to a sequence modification polynucleotide.

As is provided herein, in some embodiments, the present disclosure describes the development and use of a polymeric modification agent (e.g., blocking agent) that can bind strongly enough to a polynucleotide molecule, e.g., a DNA molecule, such that a process (e.g., replication) is temporarily slowed or stalled. In some such embodiments, a single-stranded polynucleotide (e.g., a lagging strand of DNA).

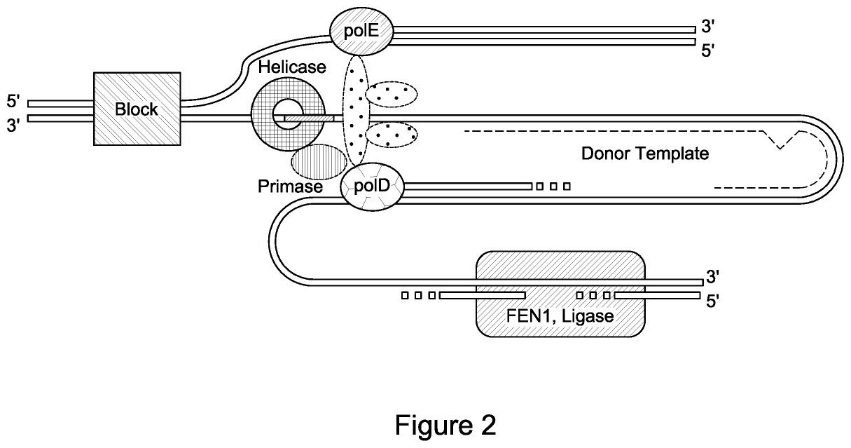

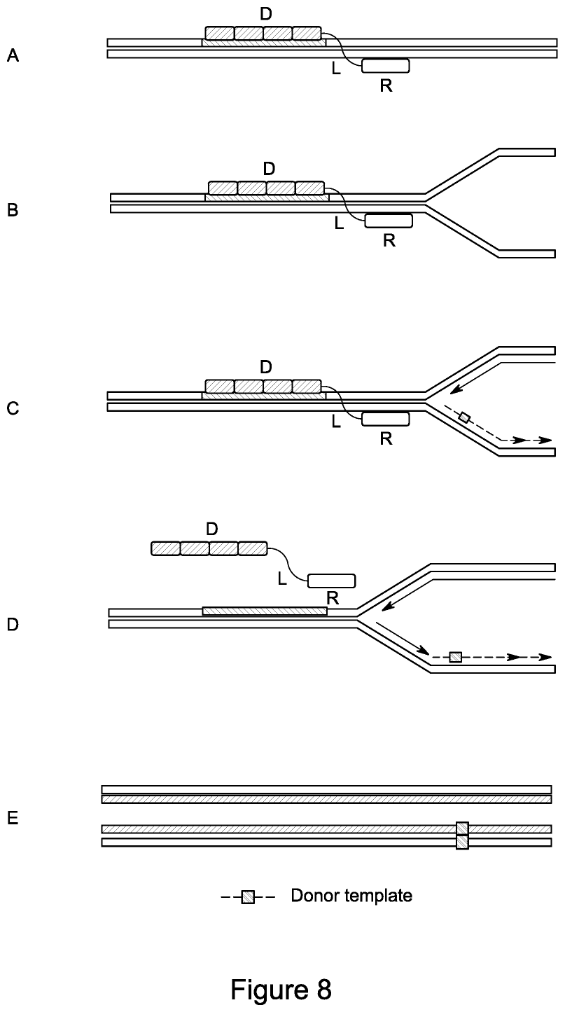

Thus, by way of non-limiting example, in some embodiments, the present disclosure provides a D element of a DNA sequence specific “blocking” agent (e.g., a DLR molecule) can bind strongly enough to a single strand of DNA such that a replication fork is temporarily slowed or stalled. In some such embodiments, a single stranded DNA segments is exposed and another polynucleotide such as an R-element can bind to the opposite strand from where the D element is bound (see, e.g., FIGS. 2 and 8A-C).

### Nucleotide Conversion Strategies

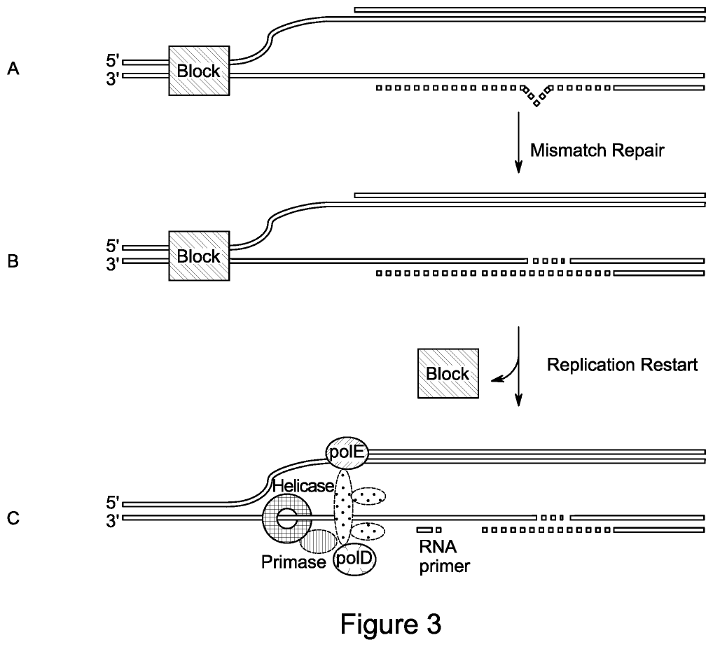

In some embodiments, the present disclosure provides technologies (e.g., systems, compositions, methods, etc.) such that standard processes of mismatch repair (e.g., including genes and factors such as XRCC1, MSH2, etc.) and DNA replication restart (e.g., CDC45), as are known to those of skill in the art, enable, e.g., DNA conversion, progression of DNA replication and cell division, resulting in gene conversion (e.g., via a sequence modification, e.g., substitution, deletion, addition) in some daughter cells (FIG. 3).

### Mismatch Repair

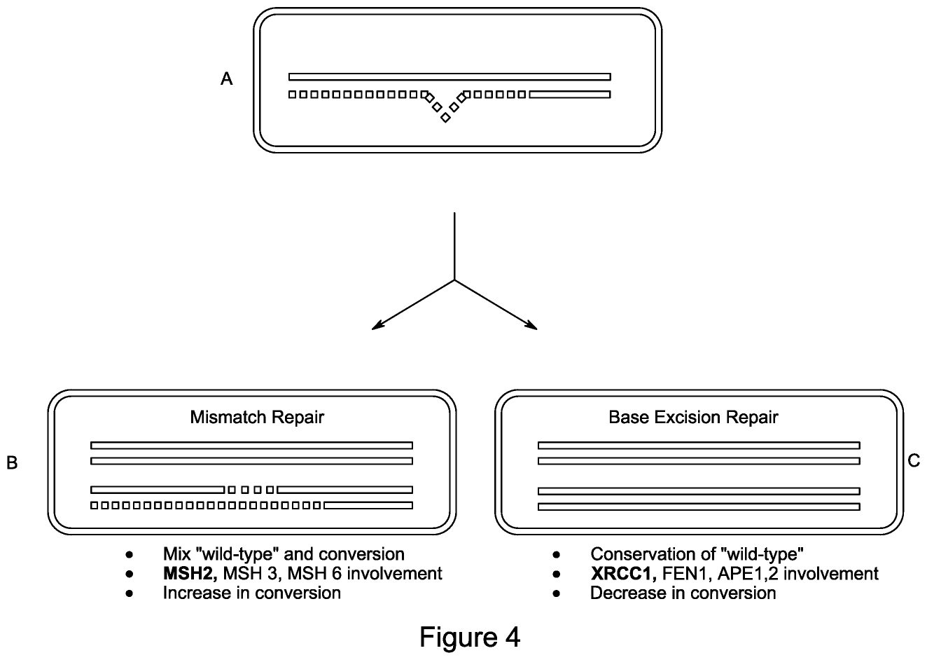

For example, base pair mismatches can be repaired by a number of DNA repair mechanisms, including mismatch repair and/or base excision repair/nucleotide excision repair. A key component of mismatch repair is MSH2 and reduction of levels of MSH2 in a cell can result in a lower frequency of mismatch repair and consequently a reduction of DNA conversion. A key factor for base excision repair and/or nucleotide excision repair is XRCC1. However, base excision repair/nucleotide excision repair has been reported to favor conversion to an “original” nucleotide sequence; thus, such an approach on its own may reduce likelihood that nucleotides derived from a sequence modification polynucleotide (e.g., a correction polynucleotide) will successfully result in a new polynucleotide sequence (e.g., a new DNA sequence) in daughter cells relative to a sequence in a parental cell prior to a genetic modification. The present disclosure recognizes that combining aspects of different repair approaches, e.g., base excision repair, etc., may increase DNA conversion frequencies. For example, without being bound by any particular theory, in some embodiments reduction of levels of a base excision repair factor, e.g., XRCC1, may reduce frequencies of base/nucleotide excision repair and, accordingly, increase DNA conversion frequencies. Thus, in some embodiments, the present disclosure provides technologies (e.g., systems, methods, compositions, etc.) that can modify (e.g., increase) gene conversion can by influencing levels of one or more DNA mismatch repair factors (e.g., MSH2, e.g., XRCC1) (see FIG. 4).

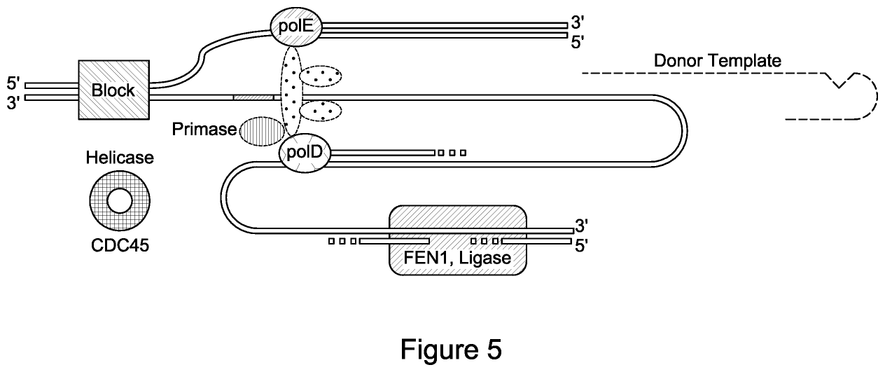

Replication fork restart may occur in cases where, e.g., DNA replication has been temporarily slowed or stalled. In some embodiments, the present disclosure recognizes that in situations where DNA is the polynucleotide being modified, increases in rates of DNA conversion may be achieved by influencing one or more cellular levels of replication fork restart molecules (e.g., CDC45). The present disclosure provides the insight that, in some embodiments, if a replication fork restart process occurs (i.e., after temporarily slowing or stalling) before a sequence modification polynucleotide is able to bind, e.g., to a lagging strand, then gene conversion will not take place. Thus, the present disclosure provides a new mechanism to improve efficacy of gene conversion by reduction of levels of replication fork restart molecules. Accordingly, in some embodiments, as reducing levels of CDC45 in a cell can reduce or slow down replication fork restart and thus increase gene conversion frequencies (see, e.g., FIG. 5).

### Uses of Inhibitory Nucleic Acid Approaches

In some embodiments, a reduction or an increase of specific factors involved in various DNA repair processes can influence gene conversion rates (see, e.g., Example 10). Thus, in some embodiments, changing cellular levels of certain factors involved in DNA repair is useful both as a technological means to influence conversion frequencies as well as it can help to further elucidate details of mechanisms involved in gene conversion using a RITDM system.

In some embodiments, gene conversion is influenced by changing cellular levels of factors involved in mismatch repair (for example, MSH 2), base excision repair and/or nucleotide excision repair (for example, XRCC 1) and/or replication fork restart (for example CDC 45). The present disclosure contemplates that, in some embodiments, influencing cellular levels of other factors involved in these or other DNA repair pathways will influence DNA conversion rates.

In some embodiments of this disclosure other means can be used to enhance DNA conversion, such as influencing cell culture conditions (e.g., by heat or cold shocks and/or depletion or access of certain cell medium components). Other compounds that influence activity of DNA repair components (without necessarily influencing their cellular levels) can potentially be used as enhancing agents.

### RITDM Efficiency

In some embodiments, a RITDM system provides methods of a targeted genetic (e.g., DNA) modification. As described herein, targeted genetic (e.g., DNA) modifications are, but are not limited to, changes that include insertions, deletions and/or substitutions (e.g., point mutations). In some embodiments these methods may include transfection of a cell with a RITDM system. In some such embodiments, a RITDM system comprises both a DLR and a sequence modification polynucleotide in accordance with the present disclosure.

In some embodiments, the present disclosure provides RITDM-based methods comprising a DLR agent and a sequence modification polynucleotide. In some such embodiments, a RITDM system is capable of efficiently generating an intended nucleic acid modification at a target site, while limiting formation of off-target mutations. For example, in some embodiments, ingle cellular clones of the present disclosure show on-target gene conversion without significant off-target effects (see, e.g., Example 3). Certain characteristics of RITDM provide for extremely low risk in gene editing (i.e., low risk of off-target events) and, accordingly, provide increased safety for development of therapies applicable for use in human subjects.

In some embodiments, the present disclosure recognizes that a RITDM system, as provided herein is capable of modifying a nucleic acid sequence with a low incidence of indels. An “indel”, as used herein, refers to an insertion or deletion of (a) nucleotide base(s) within a nucleic acid. Such insertions or deletions can lead to frame shift mutations within a coding region of gene.

In some embodiments, it is desirable to combine a DLR agent (e.g., a DLR molecule) with a sequence modification polynucleotide (e.g., a donor template) to efficiently make desired genetic modifications with extremely low incidences of undesired indels in such a nucleic acid. In some embodiments, a RITDM system is capable of generating a desired gene conversion while achieving (much) lower percentages of indels at a target site than would be obtainable with methods that other available methods (e.g., those making use of nucleases to generate breaks in a polynucleotide chain). In some embodiments undesirable indels frequencies are obtainable at frequencies lower than 1%, ranging from 0.05% to 1%, similar to frequencies observed in an untargeted background. Frequencies and numbers of desired genetic (e.g., DNA) modifications and undesired mutations and indels may be determined using any suitable method, for example by methods used in examples below.

### DNA Replication, Uses and Modifications Thereof

As described herein, DNA replication involves creation two copies of a single, “original” sequence from genetic material in a cell; this is typically associated with the process of cell division and forms the basis of genetic inheritance.

### Cell Synchronization at G1 S Boundary (Prior to DNA Replication)

In some embodiments, the present disclosure provides technologies that recognize and make use of certain advantageous features of DNA replication. For example, in some embodiments, synchronization of cells to a specific stage is useful. For instance, one example of such a synchronization method makes use of thymidine as inhibitor for cell cycle progression through the G1/S boundary, prior to DNA replication (Chen and Deng. 2018. Bio Protoc 8 17-23, which is herein incorporated by reference in its entirety). In some embodiments, cells can be synchronized by a single or double thymidine block protocol. Other experimental methods to synchronize cells may also be used and will be known to those of skill in the art.

### Transcription Modification

The present disclosure also recognizes that one challenge limiting genomic engineering is difficulty in precisely targeting gene regulation approaches. For example, in some embodiments, the present disclosure provides technologies that specifically target a polymeric modification agent to a precise location in order to downregulate a particular activity such as gene transcription.

Consistent with technologies of the present disclosure as described herein, another key aspect is ability to achieve gene regulation (i.e., genomic engineering) without having to introduce one or more breaks in a polynucleotide (e.g., a gene). For example, in some embodiments the present disclosure provides one or more agents to achieve such gene regulation. In some embodiments, an agent is a sequence-specific binding molecule (e.g., a polymeric blocking agent, e.g., a DLR molecule) that does not use an additional sequence modification polynucleotide as in the RITDM approach. In some such embodiments, a polymeric modification agent without another agent such as a sequence modification polynucleotide, can be introduced into a cell to achieve gene regulation (e.g., transcriptional repression or silencing) and, as with the RITDM system, do so without the administered agent creating single- or double-stranded breaks in endogenous polynucleotides (e.g., DNA, RNA, etc.).

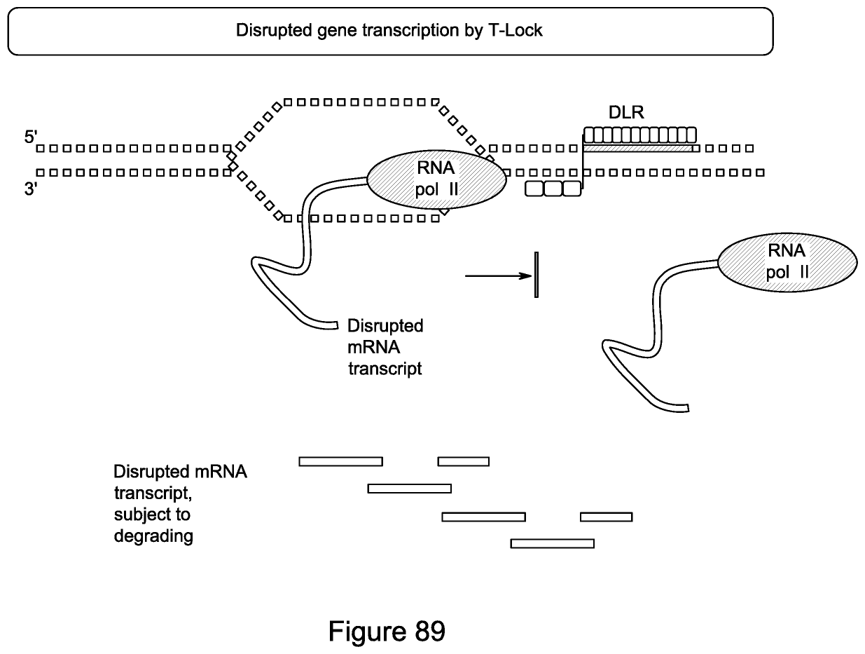

In some embodiments a cell is contacted with a polymeric modification agent (e.g., a polymeric blocking agent, e.g., a DLR molecule) to genomically engineer a target. For example, in some embodiments, a DLR molecule is capable of binding to a polynucleotide that is being transcribe. In some such embodiments, the binding or association of the DLR molecule with the polynucleotide disrupts the activity of, for example, an RNA polymerase, resulting in dissociation of the RNA polymerase and subsequent breakdown of the partially transcribed mRNA. In some such embodiments, a DLR molecule is engineered by combination of various elements providing a sequence-specific DNA binding activity at a target sequence in a genome. In some such embodiments, a DLR molecule is capable of annealing or otherwise associating to a polynucleotide (see, e.g., FIG. 89) and disrupting transcription at a target site, e.g., in a genome. In some embodiments a polymeric modification agent, e.g., a blocking agent (e.g., a DLR agent, e.g., a DLR molecule) will be administered to and/or administered to a cell.

In some embodiments, in addition to a polymeric modification agent (e.g., blocking agent) an enhancing or inhibiting agent (e.g., an siRNA, etc.) may also be administered. In some embodiments, such an enhancing or inhibiting agent is only administered with a polymeric modification agent in the presence of a sequence modification polynucleotide. In some embodiments, more than one modification agent (e.g., blocking agent) and/or enhancing or inhibiting agent, (e.g., siRNA) may be administered to and/or presented to a cell.

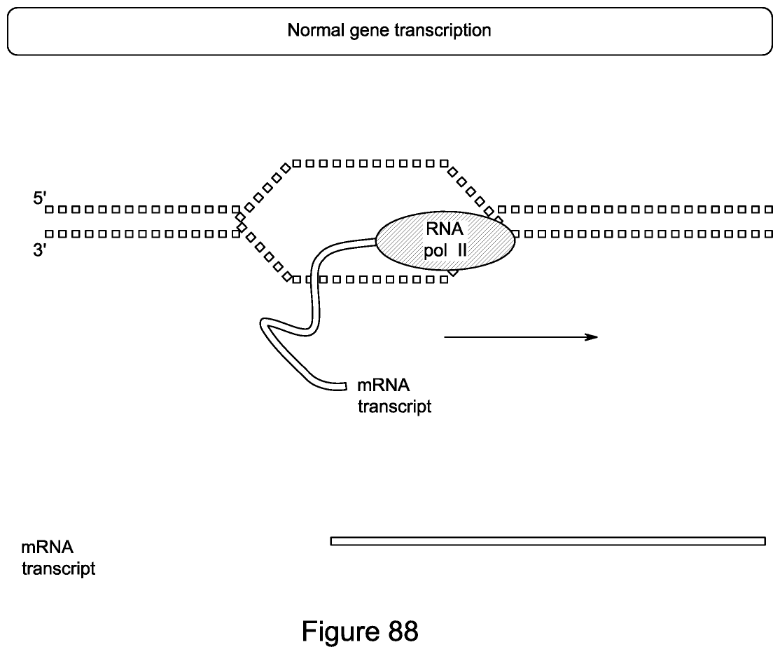

As will be understood by those of skill in the art, gene transcription is a process by which genetic information encoded in a polynucleotide (e.g., a strand of DNA) is copied into messenger RNA (mRNA). Transcription is carried out by an enzyme called RNA polymerase (RNAP) along with one or more accessory proteins called transcription factors, collectively referred as transcriptional machinery (Hahn, S. Nat Struct Mol Biol 2004; 11: 394-403, which is herein incorporated by reference in its entirety). As depicted in FIG. 88, transcription is initiated and RNAP moves along a DNA strand and begins mRNA synthesis by matching complementary bases to those of the DNA. Once mRNA is completely synthesized, transcription terminates. Newly formed mRNA copies of a gene then serve as blueprints for protein synthesis during the process of translation.

As will also be understood by those of skill in the art, RNAP progression may pause, stall, or be otherwise disrupted upon encountering any number of situations or “roadblocks” during movement of the polymerase along the DNA strand. A potential consequence of a stalled, paused, or otherwise disrupted RNAP activity is that transcription can be terminated immaturely, resulting in ineffective or incomplete mRNA synthesis. Generally, incomplete mRNA will not result in protein synthesis and, if it does, will not produce full-length or functional protein. Rather, it is more likely that RNAP disruption and dissociation from the DNA strand will result in mRNA that gets degraded.

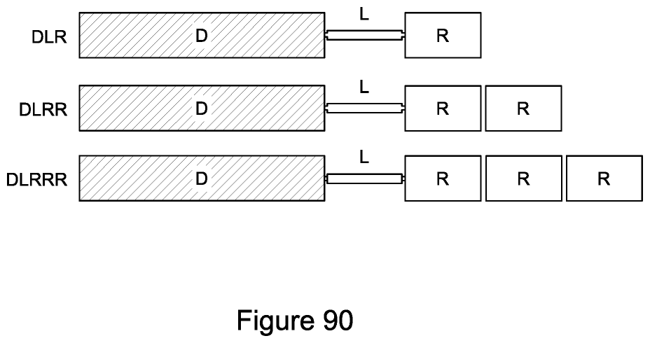

The present disclosure provides, among other things, technologies to perform gene regulation (e.g., suppress gene expression, e.g., by site specific disruption of transcription) using polymeric blocking agents (e.g., DLR molecules). Without being bound by any particular theory, the present disclosure contemplates that a DLR molecule may be further modified to increase DNA binding capacity and, thus, used to impact one or more aspects of gene regulation. For example, in some embodiments, the present disclosure contemplates that combining site-specific targeting with strengthened binding of a DLR molecule by adding one or more additional R elements to a molecule of the formula D-L-R, will facilitate gene regulation (e.g., via disruption of transcription, e.g., by interference with transcriptional processes). For example, in some embodiments, two or three R elements can be tethered together to enhance DNA binding (see FIG. 90, which illustrates several exemplary DLR molecules with one, two, or three R elements). Linked R elements can be used for gene regulation application can be multiples of the same or different R units. Thus, by way of non-limiting example, in some embodiments, when a DLR binds to a specific polynucleotide (e.g., DNA) target, it can block gene transcriptional complexes, interfering with RNAP progression along a polynucleotide (e.g., a gene), thereby disrupting transcription and ultimately reducing mRNA transcript levels.

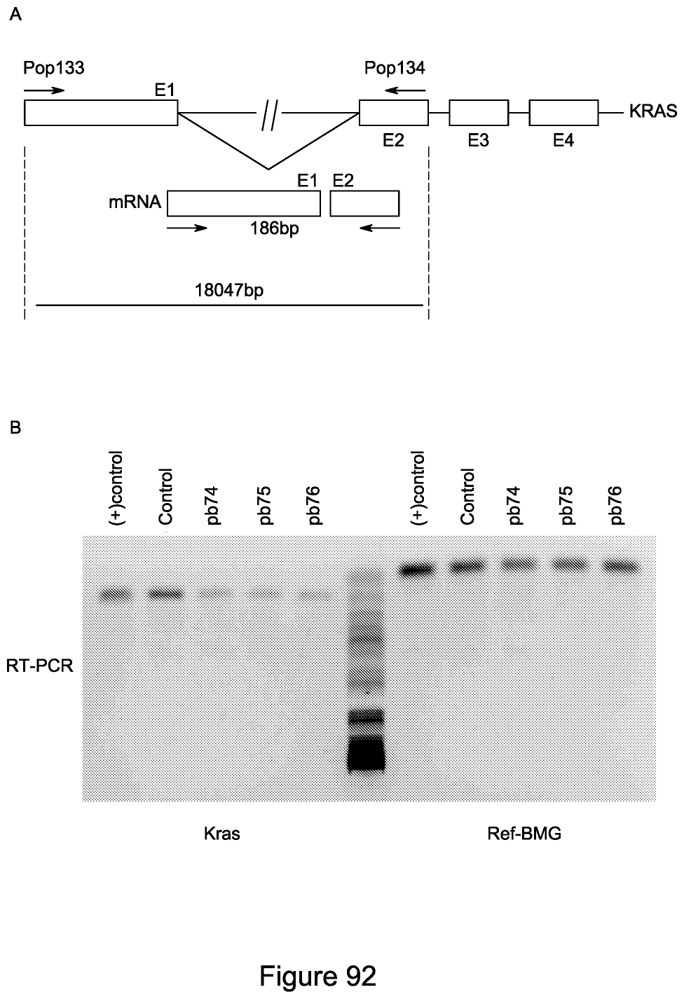

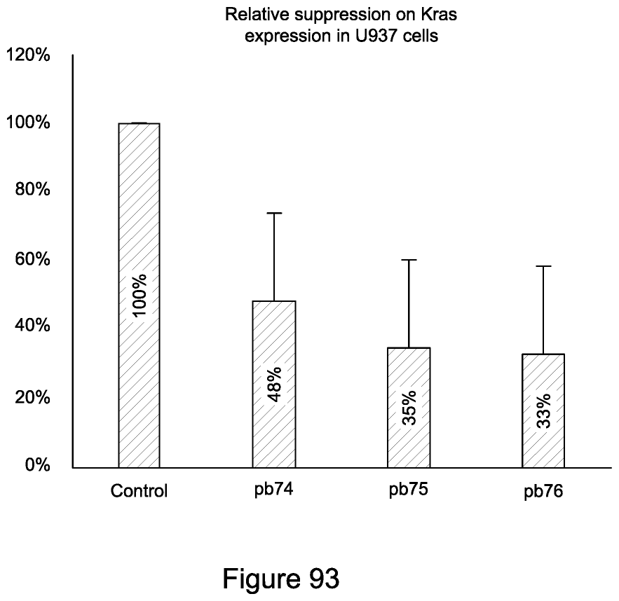

In some embodiments, a DLR molecule can bind to a target site of a polynucleotide (e.g., in a genome). During gene expression, contact of a cell by a DLR molecule such as a DLR molecule with increased DNA binding capacity, can create a situation where RNAP encounters a DLR molecule bound to DNA at the target site. By way of non-limiting example, the DLR molecule can then block the RNAP from continuing to transcribe the DNA. Without being bound by any particular theory, the present disclosure contemplates that upon transcription interruption, incompletely transcribed mRNA can then be subject to degradation. As a consequence, transcribed full-length mRNA from a target is reduced. FIGS. 88 and 89 depict mRNA transcription in presence and absence of exemplary DLR molecules. FIG. 88 illustrates mRNA transcription of a DNA strand by RNAP. FIG. 89 illustrates an exemplary DLR molecule binding to target sequence, thereby obstructing RNAP from moving along the same DNA strand. Consequently, in the presence of a sequence-specific DLR molecule, transcription is downregulated as evidenced by reduced mRNA transcripts detected (see, e.g., FIGS. 92A and 92B and FIG. 93).

Accordingly, the present disclosure provides the insight that developing technologies (e.g., systems, compositions, methods) to slow, stall, or otherwise disrupt a polynucleotide process such as transcription can regulate a gene in a sequence-specific manner to specifically reduce mRNA transcription of one or more targets. Thus, for example, in some embodiments, disruption of RNAP activity from a DNA strand that is being transcribed results in reduced mRNA production which, may, in some embodiments, reduce protein levels and/or function of one or more genes.

The present disclosure recognizes that, among other things, it would be advantageous to be able to achieve precise control over genetic activities (e.g., genomic engineering, e.g., gene regulation, e.g., gene transcription) without needing to introduce one or more breaks into genetic material (e.g., DNA, RNA, mRNA, etc.). To implement such programmed gene regulation at a target, DLR molecules are introduced into cells in formats of DNA plasmids, RNA molecules, and/or proteins with or without modifications.

As described and demonstrated herein, in some embodiments, polymeric modification agents such as DLR molecules can be used to modify and/or regulate one or more targets. For instance, without being bound by any particular theory, the present disclosure contemplates that polymeric modification agents can change (e.g., slow, disrupt, terminate) transcription. Surprisingly, when polymeric modification agents (e.g., DLR molecules) are designed and engineered in certain ways, such as having one, two, three or more R-elements, they can also achieve targeted programmed gene regulation (e.g., suppressing transcription) without any substitutions, deletions, additions, etc. as in RITDM which combines a polymeric modification agent and sequence modification polynucleotide. For example, in some embodiments, DLR molecules can be used to suppress or silence transcription. That is, without wishing to be bound by any particular theory, the present disclosure contemplates that a polymeric modification agent can interfere with transcription during gene expression. For instance, in some embodiments, a polymeric modification agent can interfere, in a sequence-specific manner, with RNA polymerase activity and cause an RNA polymerase to dissociate from a polynucleotide strand, thus causing mRNA production to stop and result in breakdown of incompletely transcribed mRNA.

### Compositions

Among other things, the present disclosure provides compositions. In some embodiments, a composition comprises an agent as described herein. In some embodiments, an agent is a blocking agent (e.g., a polymeric modification agent, e.g., a DLR molecule). In some embodiments, an agent is a modification agent (e.g., a sequence modification agent, gene regulation agent, transcription modification agent, an enhancing agent, an inhibiting agent, etc.). In some embodiments, a composition comprises one or more blocking agents and/or sequence modification agents as described herein. In some embodiments, a composition comprises a plurality of blocking agents and/or modification agents (e.g., sequence modification polynucleotides).

In some embodiments, a composition comprises a polynucleotide encoding a polymeric modification agent or a portion thereof. In some embodiments, a composition comprises a polymeric modification agent comprising a sequence encoding a DLR molecule or a portion thereof.

In some embodiments, a composition comprises an agent encoding a sequence modification agent (e.g., a correction template, a donor template). In some embodiments, a composition comprises an agent comprising a sequence encoding an enhancing and/or inhibiting agent, e.g., an siRNA, or portion thereof. In some such embodiments, an enhancing agent and/or inhibiting agent is used to, e.g., modify cellular machinery such as, for example DNA replication machinery.

In some embodiments, a composition comprises at least two agents, e.g., a polymeric modification agent and a sequence modification agent, or at least three agents, e.g., a polymeric modification agent, a sequence modification agent, and an enhancing agent/inhibiting agent, etc.

In some embodiments, a composition comprises a cell.

In some embodiments, a composition is or comprises a construct or a vector. In some such embodiments, a construct or vector can encode one or more agents or portions thereof, as described herein.

In some embodiments, a composition is or comprises a pharmaceutical composition.

### Modification Agents

The present disclosure appreciates that in some embodiments, it may be advantageous to develop a strategy in which a polynucleotide (e.g., DNA) may be modified without inducing one or more breaks in a given polynucleotide molecule. For example, the present disclosure provides the insight that if, for example, DNA replication is able to be slowed at a particular point, there would be enough time for a genetic modification (e.g., substitution, deletion, addition) to be made in, e.g., a lagging DNA strand, such that no breaks would need to be introduced into a molecule comprising target site. Without being bound by any particular theory, the present disclosure contemplates that one way to achieve a genetic modification without inducing a break is, for example, to make a modification at a target site by providing an agent that associates (e.g., binds) at or near a landing or target site and also provides another molecule which acts as a template or donor to achieve a nucleotide change.

### Polymeric Modification Agents

In some embodiments, the present disclosure provides a polymeric modification agent. In some embodiments, a polymeric modification agent is or comprises a DLR molecule. In some such embodiments, a DLR molecule binds to a binding site. In some such embodiments, a binding site may the same the target site. In some embodiments, a binding site overlaps (i.e., shares one or more nucleic acid residues) with a target site. In some embodiments, binding site and a target site do not overlap at all.

In some embodiments, a polymeric modification agent is a blocking agent. In some such embodiments, a blocking agent is engineered to, for example, reversibly bind to a nucleotide sequence (e.g., a landing site, a binding site, etc.), in a sequence-specific manner. In some embodiments, a blocking agent is an agent that is or comprises one or more components that bind(s) to a landing site, binding site, and/or target site. In some embodiments, a blocking agent comprises a component that, e.g., slows or stalls DNA replication, RNA transcription, mRNA translation, etc. In some embodiments a blocking agent is or comprises a DLR molecule, as provided herein.

### DLR Molecules and Architecture

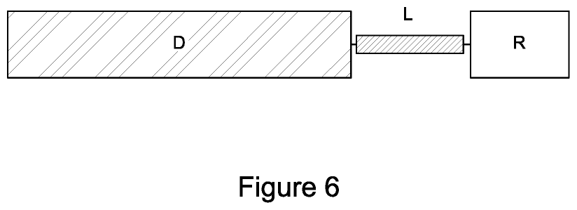

In some embodiments, an agent is or comprises a DLR molecule (see, e.g., FIG. 6). In some embodiments, a DLR molecule has or comprises a structure set forth as D-L-R. The present disclosure also provides, among other things, methods of making and using disclosed agents and/or molecules. In some such embodiments, a DLR molecule reversibly binds to double-stranded DNA, in a sequence specific manner. In some embodiments, a DLR agent comprises at least two elements: at least one “D” and at least one “R”, with an optional “L” element. In some embodiments, a DLR molecule may be ordered with D, L, and R elements placed consecutively. Thus, as described herein, in some embodiments, a DLR molecule can be schematically represented as D-L-R or R-L-D.

In some embodiments, a given DLR molecule may have more than one each of a given D, L, or R element. For example, in some embodiments, a D element may be fused or otherwise connected to one or more L elements, which may each be fused or otherwise connected to one or more R elements. In some embodiments, a given DLR molecule may have two R elements, three R elements, four R elements or more. In some embodiments, a given DLR molecule may have two L elements, three L elements, four L elements, or more. In some embodiments, a DLR molecule may be schematically represented as, e.g., D-L-R; D-L-R—R; D-L-R—R—R, etc.

In some embodiments, a D element is comprised of multiple components or DNA binding elements. For example, in some embodiments, a D element is “hybrid” comprising zinc-finger nuclease components and additional sequences. As provided herein, “D” is a first domain comprising a sequence-specific DNA binding element that binds to one DNA strand; “L” is an optional linker element between segments “D” and “R”; and “R” is a second domain that comprises a sequence-specific or non-sequence-specific DNA binding element that can bind to the corresponding, opposite DNA strand to which a D element binds. In some embodiments, an R element is or comprises a polynucleotide that binds to a different polynucleotide than a D element. In some such embodiments, an R element is bound to a complementary polynucleotide on the same molecule as a D element. In some embodiments, an R element is bound to a polynucleotide on a different molecule as a D element of a single DLR molecule. In certain aspects the three elements are able to be reversibly bound (element D and R) or associated (element L) to a polynucleotide (e.g., DNA, e.g., RNA) molecule.

In some embodiments a DLR molecule may be or comprise a polypeptide. In some such embodiments, where a DLR is a polypeptide, a D element can be located at either an N-terminal or C-terminal portion of a polypeptide, with an R-element located at an opposite location (e.g., C-terminal or N-terminal location). In some embodiments, where a DLR molecule (e.g., polypeptide) comprises one or more L elements, such L elements are located in between D elements and R elements.

As described herein, technologies provided by the present disclosure (e.g., systems, methods, compositions, etc.) achieve one or more genetic modifications at one or more target sites. Accordingly, for example, in some embodiments, a DLR molecule binds at a target site in a target genome wherein a D element binds to one strand of a DNA double helix in a sequence-specific manner and an R element binds to the opposite DNA strand (see, e.g., FIG. 8A-8C). Then, when DNA replicates, such a DLR molecule is designed that it can interfere with replication fork progression at a target site (e.g., via stalling or slowing). In some such embodiments, when a sequence modification polynucleotide is present (such as illustrated in, e.g., FIG. 8 where a single stranded oligonucleotide has a desired DNA modification), the sequence modification polynucleotide can anneal to its complementary strand and create a sequence mismatch (FIG. 8D). In some embodiments one or more intrinsic DNA repair processes in a given cell can result in a genetic modification by incorporating the desired alteration (e.g., the sequence of the sequence modification polynucleotide). Thus gene editing can be accomplished without having to induce or cause, e.g., a DNA strand break with nuclease activity of a DLR molecule itself (see, e.g., FIG. 8E).

In some such embodiments, a DLR molecule comprises a first domain, an optional linker, and a second domain. In some embodiments, a first domain is capable of binding to a DNA sequence (e.g., a D element, e.g., a zinc finger protein or a Cas9 protein), and a second domain (e.g., an R element) is able to bind to a polynucleotide (e.g., a DNA double helix), for example, on the strand opposite of that to which the first domain can bind or to another strand on another molecule. In some such embodiments, a first domain binds in a sequence-specific manner and a second domain binds in a non-sequence specific manner. In some embodiments, a second domain binds in a sequence specific manner. In some embodiments, binding of a DLR molecule can result in stalling or slowing of cellular machinery (e.g., replication machinery, transcription machinery, etc.). For example, in some embodiments, in the context of DNA as a target site, binding of such a DLR molecule can result in stalling or slowing of the replication fork and thus enabling a polynucleotide to bind to exposed single stranded DNA sequences. For example, in some embodiments, when a polynucleotide contains one or more nucleotides that are different from that of an original host cell, this may result in DNA conversion. The present disclosure contemplates that, in some embodiments, DLR molecules as described herein may be useful for targeted editing of a polynucleotide (e.g., DNA, RNA, etc.) without directly or indirectly causing single or double stranded breaks at or near a target site.

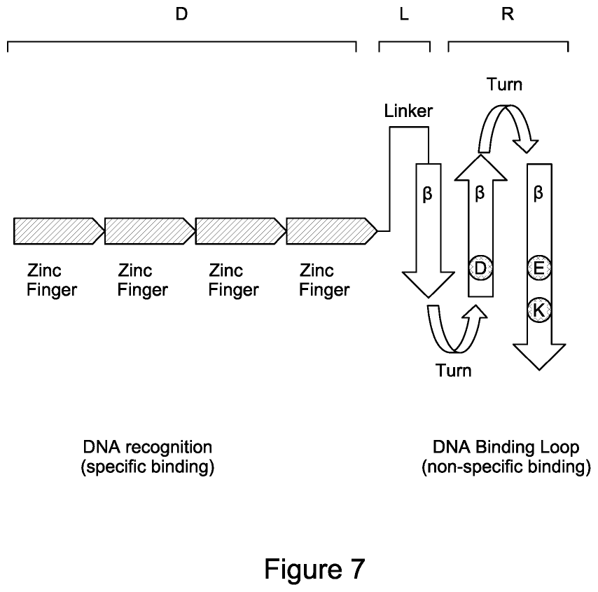

In some embodiments a DLR molecule can be or comprise a polypeptide (e.g., a protein). For example, a DLR molecule, may, in some embodiments, comprise a D element comprising an array of 4 zinc fingers that can recognize a target site (e.g., a DNA target site) and an R element may be or comprise 3 anti-parallel beta sheets that can create a three-dimensional structure that can interact with DNA molecules in a non-sequence specific manner (see, e.g., FIG. 7). In some embodiments, such a DLR molecule is based on a structure from a core fold found in PD-(D/E)XK nuclease structures where D, E and K are critical amino acid residues resides in DNA cleavage activity. In some embodiments, genetic modification of one or more of these residues is done to abolish DNA cutting activities.

### “D” Elements

In some embodiments, the present disclosure provides a DLR molecule, which comprises a D-element, which element is a domain capable of binding to a sequence (e.g., a nucleotide sequence, e.g., a landing site, e.g., a binding site) specifically on a single strand of a polynucleotide (e.g., such as a single strand of a DNA molecule, or on an RNA transcript, etc.). In some embodiments, a D element is or comprises, for example, zinc-finger proteins, catalytically inactivated Cas9 (“dCas9”), or other nucleotide (e.g., DNA) binding proteins. By way of non-limiting example, a D element may be or comprise one or more Zinc Finger proteins or domains; TALE-proteins or domains; Helix-loop-helix proteins or domains; Helix-turn-helix proteins or domains; CAS-proteins or domains; Leucine Zipper proteins or domains; beta-scaffold proteins or domains; Homeo-domain proteins or domains; High-mobility group box proteins or domains or characteristic portions thereof or combinations and/or parts thereof.

The present disclosure also provides the surprising finding that a D element may be or comprise more than seven zinc finger modules. As will be understood by those of skill in the art, working with and using zinc finger arrays can present several technological and methodological challenge. By way of non-limiting example, the present disclosure provides a DLR molecule, wherein the D element comprises 11 zinc finger modules. In some embodiments, such a DLR molecule is used to successfully modify genetic material in a cell (e.g., a base change in a target sequence of a cell).

In some embodiments, a D element is or comprises a sequence specific recognition element. In some such embodiments, a D element can be designed to not only recognize a specific sequence, but also to bind to that specific sequence within a context of a certain genome. For example, in some embodiments, a D-element is or comprises an array of 4 zinc-finger modules, each of which is designed to recognize a 3-nucleotide sequence (see, e.g., FIG. 7). For example, in some such embodiments a target site is a 12-nucleotide sequence.

In some embodiments a designed binding sequence (e.g., a sequence that binds to, e.g., a binding site and/or a landing site) can range from 9 nucleotides (e.g., when using 3 zinc finger domains) to larger than 33 nucleotides in length (e.g., using 11 or more zinc-finger modules). In some embodiments a D element can be or comprise a designed zinc finger array, containing a number of zinc fingers (e.g., 1, 2, 3, 4, 5, 6, 7, 8, 9, 10, 11, 12, 13, 14, 15 etc.), wherein each zinc finger is designed to recognize and bind three consecutive nucleotides. For example, if a target site (e.g., on a target molecule, e.g., a target DNA strand, on RNA molecule e.g., an RNA molecule with loop structure and base pairing, etc.) is 9 bp in length, a D element can be designed to be or comprise three zinc finger arrays. If, for example, a target site is 33 bp in length, then a D element can be designed to be or comprise eleven zinc fingers.

In some embodiments a D element is or comprises a sequence specific DNA recognition element that is engineered not only to recognize a specific sequence, but also to bind to that specific DNA sequence (e.g., target site) with sufficient affinity (e.g., sufficient affinity to slow or stall a process, e.g., a DNA replication process, e.g., a transcription process, etc.).

In some embodiments, a D element can also be or comprise naturally occurring or designed factors with ability to provide both sequence specific recognition and binding. For example, in some embodiments a D element can be or comprise a dCas9 protein associated with a specific guide RNA, a Transcription Activator-Like Effector domain (TALE), etc.

In some embodiments a DLR molecule may be encoded in, e.g., DNA, RNA, chemically modified, and/or or synthetic nucleotides. In some embodiments, a given DLR molecule can be or comprise a D element at the 5′ end or at the 3′ end of a given molecule.

In some embodiments, D elements are binding elements that are typically folded macromolecules that adapt a 3D structure that recognizes a double or single-stranded polynucleotide (e.g., a DNA molecule). In some embodiments, a D-element is at least 9 nucleotides in length.

In some embodiments D elements can be engineered or designed such that a polynucleotide (e.g., DNA) recognition sequence is different from that of an original or a naturally occurring polynucleotide (e.g., DNA) binding element. In some embodiments a D element can be designed such that it binds with higher affinity and/or selectivity to a sequence that is, in at least one nucleotide, changed compared to an original polynucleotide binding sequence. In some embodiments a D element can be engineered, designed or selected to recognize a specific sequence (e.g., a DNA sequence, an RNA sequence, e.g., an mRNA sequence, etc.). In some embodiments a D element can be designed, engineered and/or selected to have high or low binding affinity for a specific sequence (e.g., a target sequence, e.g., a DNA sequence, an RNA sequence, etc.). In some embodiments a D element can be designed, engineered and/or selected to have high or low affinity for non-sequence specific DNA binding. In some embodiments binding affinity can be measured in vitro, mimicking conditions that are similar to in vivo conditions in a cell. In some embodiments binding affinity and/or selectivity can be measured in vitro using assays known to those of skill in the art such as e.g., DNA-protein interaction assays. In some embodiments sequence selectivity can be measured in vitro, mimicking conditions that are similar to in vivo conditions in a cell. In some embodiments affinity and selectivity can be measured in vivo using reporter-assays typical for DNA-protein interactions.

In some embodiments, sequence specificity of a D element is or comprises between about 5 to about 40 nucleotides. In some embodiments, sequence specificity of a D element is about 5-10, 10-15, 15-20, 20-25, 25-30, 30-35, 35-40 or more polynucleotides. In some embodiments, number of nucleotides involved in specificity may occur in groups of three (e.g., in zinc finger contexts, e.g., 9, 12, 15, 18, 21, 24, 27, 30, 33 or more nucleotides of specificity with each three nucleotides corresponding to one zinc finger). In some embodiments, sequence-specificity of a D element has approximately at east 15-20 nucleotides of specificity. In some embodiments, a D element has at least about 9, 10, 11, 12, 13, 14, 15, 16, 17, 18, 19, 20, 21, 22, 23, 24, 25, 26, 27, 28, 29, 30, 31, 32, 33 nucleotides of specificity (i.e., nucleotides of complementarity with a binding site target). In some such embodiments, nucleotides that are involved in sequence specificity do not need to be contiguous with one another; that is, in some embodiments, even if a D element has, e.g., 18 nucleotides of specificity with which it recognizes where to bind, those 18 nucleotides are not necessarily contiguous with one another. As will be understood to those of skill in the art and dependent upon context, in some embodiments, it may be desirable to design longer recognition sequences (e.g., longer than 15-20 nucleotides).

### Zinc Finger Proteins

Zinc finger proteins have been studied extensively. A large number of naturally occurring proteins containing zinc fingers exist in nature. In many of these proteins zinc fingers are involved in some type of interaction with nucleic acids and/or other proteins. Protein chemistry and crystal structure experiments have elucidated many aspects of zinc finger structures and mechanisms by which they can bind to other molecules. An archetypical zinc finger structure that is often involved in DNA binding and DNA sequence recognition, comprises an alpha-helix structure with two anti-parallel beta-sheets that are oriented into a three-dimensional confirmation by a coordinating zinc atom. In these structures said zinc-atom interacts with cysteine and/or histidine amino acid side chains. Specific amino acid side chains protrude from an alpha helix structure and these amino acids side chains are involved in (preferential) sequence specific binding (Choo and Klug, 1994, Proc Natl Acad Sci USA 91 11163-11167, Elrod-Erickson, et al., 1996, Structure 4 1171-1180, each of which is herein incorporated by reference in its entirety).

In some embodiments, zinc finger proteins have an ability to be used as modular units of approximately 30 amino acids, with each unit potentially able to bind to a DNA-triplet sequence. In some embodiments, zinc finger proteins can been combined into arrays of two or more zinc fingers, thus allowing for larger DNA sequences (i.e., additional DNA triplets) to be recognized and bound by Zn fingers/Zn-containing proteins (Choo and Klug, 1994, Proc Natl Acad Sci USA 91 11168-11172, which is herein incorporated by reference in its entirety).

Many sequence specific interactions between zinc fingers and DNA are known in the art. A number of studies have described how specific amino acid side chains in specific positions of alpha helices of zinc fingers allow for either more- or less-specific interactions and binding to specific nucleotides in a DNA molecule (Klug, 2010, Annu Rev Biochem 79 213-231, which is herein incorporated by reference in its entirety). Accordingly, such features may be incorporated when designing zinc finger units or zinc finger containing domains. Thus, in some embodiments, the present disclosure provides agents that incorporate zinc fingers and/or one or more features of zinc fingers that can be used to design or develop agents or approaches that preferentially recognize specific DNA sequences (Choo and Klu., 1997, Curr Opin Struct Biol 7 117-125; Klug, 2005, Proc. Japan Acad. 81 87-102; Sera and Uranga, 2002, Biochemistry 41 7074-7081, Zhu, et al. 2013. Nucleic Acids Res 41 2455-2465, each of which is herein incorporated by reference in its entirety).

In some embodiments, zinc fingers can influence behavior of adjacent zinc fingers. Accordingly, a series of preselected and pretested zinc finger dimers have been described (Isalan, et al. 1997. Proc Natl Acad Sci USA 94 5617-5621; Moore, et al, 2001, Proc Natl Acad Sci USA 98 1437-1441, each of which is herein incorporated by reference in its entirety) and a number of methods for the evaluation of interactions can be found in literature (Isalan, et al, 1998, Biochemistry 37 12026-12033, which is herein incorporated by reference in its entirety). Thus, in some embodiments, when designing or selecting zinc finger arrays for use in one or more technologies of the present disclosure, such interactions, dimers, and/or methods can be taken into consideration. The present disclosure also recognizes that zinc finger array design principles as are known in the art may not always be sufficient to accurately predict how well a given zinc finger array will work for a given purposes (e.g., as a D component of a DLR molecule used as a DNA replication stalling molecule for sequence modification). Accordingly, among other things, the present disclosure provides agents and assays that may be used to design, evaluate and optimize zinc finger arrays for use in accordance with the present disclosure.

In some embodiments a zinc finger array as described herein comprises zinc finger amino acid sequences: FQCRICMRNFS(X7)HIRTH (SEQ ID NO.2) or FACDICGRKFA(X7)HTKIH (SEQ ID NO.3). In some such embodiments, X7 represents a sequence of seven amino acids, wherein X can be any amino acids, which can be modified to enable (preferential) sequence specific binding to a specific DNA target sequence.

In some embodiments a target sequence 5′-GGGGAGGACGCGGTG-3′ (SEQ ID NO.4) is targeted by a zinc finger array that comprises a following zinc finger protein sequence: FQCRICMRNFSRSSALTRHIRTHTGEKPFACDICGRKFARSDTLTRHTKIHTGSQKPFQCR ICMRNFSDRSNLTRHIRTHTGEKPFACDICGRKFARSDNLTRHTKIHTGSQKPFQCRICM RNFSRSDHLTRHIRTHTG (SEQ ID NO.5). In some embodiments a target sequence 5′-GTGGAGCTGGACGGGGAC-3′ (SEQ ID NO.6) is targeted by a zinc finger array that comprises a following zinc finger protein sequence:

(SEQ ID NO. 7)
FQCRICMRNFSDRSNLTRHIRTHTGEKPFACDICGRKFARSDHLT
RHTKIHTGSQKPFQCRICMRNFSDRSNLTRHIRTHTGEKPFACDI
CGRKFARSDSLSEHTKIHTGSQKPFQCRICMRNFSRSSNLTRHIR
THTGEKPFACDICGRKFARSDSLTRHTKIH.

In some embodiments a target sequence 5′-GCGGCCGCCTGGTGCAGTACCGCGGCG-3′ (SEQ ID NO.8) is targeted by a zinc finger array that comprises a following zinc finger protein sequence:

(SEQ ID NO. 9)
MAAMAERPFQCRICMRNFSRSSDLTRHIRTHTGEKPFACDICGRK
FARSDTLTRHTKIHTGSQKPFQCRICMRNFSQSGDLSEHIRTHTG
EKPFACDICGRKFATSGHLTTHTKIHTGSQKPFQCRICMRNFSDS
SHLTTHIRTHTGEKPFACDICGRKFARSSHLTTHTKIHTGSQKPF
QCRICMRNFSDRSDLTRHIRTHTGEKPFACDICGRKFADRSDLTR
HTKIHTGSQKPFQCRICMRNFSRSDTLTRHIRTHTG.

In some embodiments, a target sequence 5′-CTGGCAGTGTACCAGGCCGGGGCCCGCGAGGGC-3′ (SEQ ID NO.10) is targeted by a zinc finger array that comprises a following zinc finger protein sequence:

(SEQ ID NO. 11)
MAAMAERPFQCRICMRNFSDRSHLTRHIRTHTGEKPFACDICGRK
FARSDNLTRHTKIHTGSQKPFQCRICMRNFSDSSHLSEHIRTHTG
EKPFACDICGRKFADRSDLTRHTKIHTGSQKPFQCRICMRNFSRS
DHLTRHIRTHTGEKPFACDICGRKFADRSDLTRHTKIHTGSQKPF
QCRICMRNFSRSDNLSEHIRTHTGEKPFACDICGRKFAESSNLTT
HTKIHTGSQKPFQCRICMRNFSRSSSLTRHIRTHTGEKPFACDIC
GRKFAQSSDLTRHTKIHTGSQKPFQCRICMRNFSRSDSLSEHIRT
HTG.

### Cas9 Proteins

Cas9 (CRISPR associated protein 9) has been used in a wide variety of gene editing and genome engineering applications. Cas9 (and similar proteins) are found in nature and are thought to function in bacterial defense against viral infections and plasmid infections by sequence specific digestion of foreign DNA in Cas9 producing cells. CRISPR systems (Clustered Regularly Interspaced Short Palindromic Repeats system) are at the core of this bacterial adaptive host defense system, which uses sequence specific guide RNAs that can target Cas9 endonucleases to a particular target site to make breaks (e.g., double stranded breaks) in a target polynucleotide (e.g., DNA. Among other things, CRISPR/Cas9 systems have been further developed for use in gene editing and genome engineering by (i) development of synthetic guide RNAs (e.g., guides that can essentially target almost any desired polynucleotide (e.g., DNA) sequence) and (ii) by making further modifications to Cas9 endonucleases to convert them into nicking variants and/or variants that have no nuclease activity such that breaks at target sites are controlled in different ways (Cong, et al, 2013, Science 339 819-823; Jinek, et al., 2013, Elife 2 e00471, each of which is herein incorporated by reference in its entirety).

Accordingly, in some embodiments a catalytically inactive Cas9 protein may be used as a D element in a blocking agent (e.g., a DLR molecule) of the present disclosure. Dead Cas9 (dCas9) has mutations D10A and H840A relative to wild type Cas9, which abolishes ability of Cas9 to create double or single stranded polynucleotide (e.g., DNA) breaks. An exemplary dCas9 variant amino acid sequence (displayed from N-term to C-term) is SEQ ID NO: 12, listed in Table 1. In some embodiments other catalytically inactivated Cas or Cas-like proteins can be used.

### Transcription Activator-Like Effector (TALE) Proteins

Transcription Activator-Like Effector (TALE) proteins were developed as modular DNA-sequence specific binding domains. TALE protein structures, as secreted by certain Xanthomonas bacteria, can be used to design modified TALE proteins. In some embodiments, TALE proteins have DNA-binding domains with a highly conserved structure, which varies at two amino acid positions that are involved in preferred binding to specific nucleotides. Natural and designed TALE-domains that can bind preferentially to a specific 2-nucleotide sequence are known (Li, et al, 2011, Nucleic Acids Res 39 359-372, which is herein incorporated by reference in its entirety). In some embodiments, TALE-domains can be designed to be modular. In some embodiments, arrays of multiple TALE-domains can be combined to recognize longer, specific DNA sequences

### Other Sequence Specific Binding Domains

The present disclosure contemplates that in some embodiments, in addition to Zinc Fingers, Cas9 (and other Cas-like proteins), and TALE proteins, a number of other proteins, protein domains and designed proteins exist or can be developed for use as part of or as sequence specific binding domains (e.g., DNA sequence specific binding domains). These include, but are not limited to, meganucleases proteins or domains, helix-loop-helix proteins or domains, helix-turn-helix proteins or domains, Homeo-domain proteins or domains, beta-scaffold proteins or domains, High-mobility group box proteins or domains, Leucine Zipper proteins or domains and other types of naturally occurring and/or designed proteins and any combinations thereof.

In some embodiments a polynucleotide (e.g., DNA) binding element needs to be of sufficient size and structure to recognize and bind to a desired sequence. For example, in some embodiments within a context of genome editing a binding element sequence is specific within the genome of a target organism. In some embodiments, a binding element sequence is semi-specific for the genome of a target organism; for example, to be semi-specific, in some embodiments, a mammalian cell requires a sequence of at least 15 nucleotides of homology, but preferentially a larger number. In some embodiments, if a sequence-specific R element is used, sequence specificity can come from a combination of sequence specificity from a D element and an R element. That is, specificity of a given DLR molecule may be combinatorial and can come from one or more sequence-specific components of the molecule (e.g., a D element, a D element and an R element, etc.).

DLR Molecule Interaction with a Replication Fork

In some embodiments, direct interaction of a DLR molecule with components of a replication fork can occur, as illustrated in example 9. Thus, as described in example 9, interaction of a DLR molecule with a DNA replication fork opens an opportunity that a correction oligonucleotide can anneal to a (partially) complementary single stranded DNA sequence that is temporarily exposed at a replication fork. DLR binding can interfere with progression of a replication fork at in the vicinity of a DLR binding site and thus prolong exposure of a single stranded DNA conversion site.

The present disclosure contemplates that cells containing both a DLR molecule and a correction polynucleotide can thus generate a DNA conversion.

In some embodiments, agents of the present disclosure and uses thereof, e.g., DLR molecules as part of a RITDM DNA editing system are designed to lack nuclease activity. In some such embodiments, lack of nuclease activity avoids creating DNA breaks that typically result in Non-Homologous End-Joining (NHEJ). In some embodiments, when both a DLR molecule and a sequence modification polynucleotide are present in a cell, gene conversion can be achieved with only (very) low levels of background damage generated via NHEJ mediated DNA conversion processes.

In some embodiments cell synchronization (e.g., when using a thymidine block regime) enhances DNA conversion frequencies when using a DLR molecule and a sequence modification polynucleotide. In certain embodiments agents that influence cell cycle progression and/or inhibition can be used to enhance DNA modification when using a DLR molecule and a sequence modification polynucleotide.

### “L” Elements

In some embodiments, an “L element” may be optionally used to connect (link) at least one “D element” and at least one “R element.” In some embodiments, an L element comprises amino acid residues. In some embodiments provided by the present disclosure, an L element can function as a linker domain between a D and an R domain.

Though the present disclosure generally provides L elements to connect D and R elements, in some embodiments, L elements may also provide additional properties, such as, e.g., orientation of an entire DLR molecule. In some embodiments, for instance, an L element may comprise one or more components that confer additional sequence or structure specificity (e.g., addition of an Arginine to facilitate binding to G, addition of hydrophobic amino acids, addition of certain polar amino acids, e.g., lysine, which may, in some embodiments, have a greater affinity for a negatively charged molecule (e.g., DNA), etc.)

In certain embodiments, when using an amino acid linker this element can be a 4 amino-acid linker (e.g., LRGS as in SEQ ID NO.1). However, longer or shorter linkers may be used as required on a case-by-case manner. Without being bound by any particular theory, the present disclosure contemplates that a shorter linker may have certain advantages that will be understood by those of skill in the art.

In some embodiments an L element is short (e.g., 7, 6, 5, 4, 3, 2 amino acids or less) linker. In some such embodiments, a short linker has approximately 7, 6, 5, 4, 3 or fewer amino acids. For example, in some embodiments, a short linker is or comprises an amino acid sequence of LRGS (SEQ ID NO.1). In some embodiments, a linker may be or comprise a sequence of GGGSn, (SEQ ID NO: 242) wherein n is 1 or more (e.g., 1, 2, 3, 4, 5 or more) repeats.

In some embodiments, linkers comprise nucleic acid residues. In some embodiments a linker is short (e.g., 21, 18, 15, 12, 9, 6 nucleic acids or less). In some such embodiments, a short linker has approximately 21, 18, 15, 12, 9 or fewer nucleic acids. In some embodiments, nucleic acids are modified nucleic acids, e.g., locked nucleic acids, oligonucleotides, etc.

In some embodiments a linker sequence is a linker found in nature or analogous to a linker found in nature. In some embodiments, a linker is a synthetic linker. In some embodiments, a linker comprises a sequence that cannot be found in nature and has no homology to any linker found in nature. In some embodiments, a linker may be or comprise a combination of natural linkers, but arranged in patterns not found in nature, e.g., connecting one or more natural linkers that are not found in such an arrangement in nature, e.g., generating a linker comprising repeats of a natural linker, wherein the linker comprising repeats is not itself found in nature.

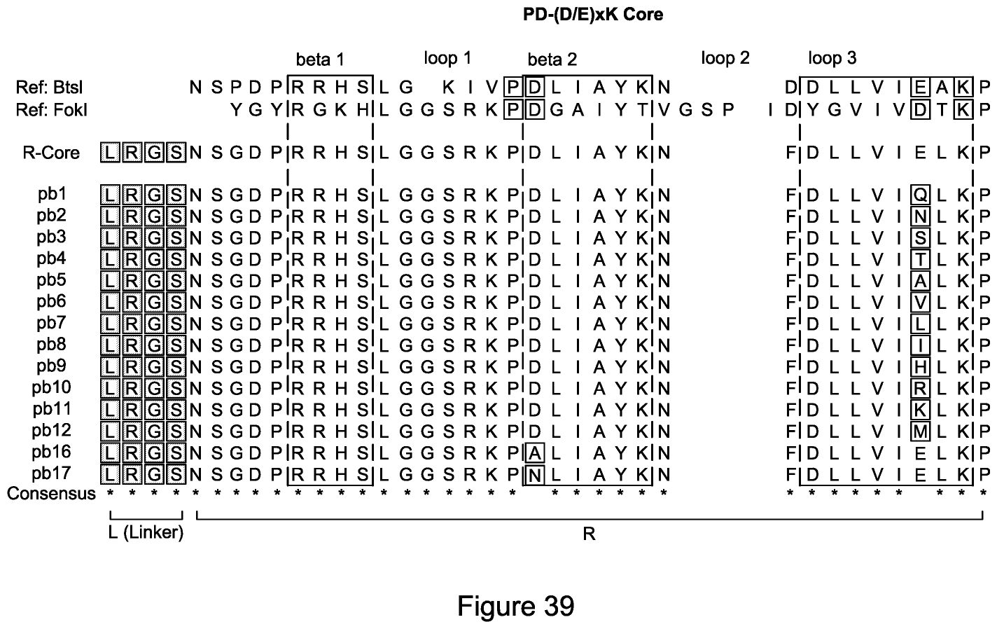

In some embodiments, a linker with a structure comprising 4-amino acids (LRGS; SEQ ID NO. 1) is used to link D and R elements. In some such embodiments, a D element is or comprises a zinc finger array in this example (see, e.g., FIG. 39).

In some embodiments, a LRGS linker (SEQ ID NO. 1) is connected to an amino acid sequence “NSGDP” (SEQ ID NO. 243) that precedes beta sheet 1 (see, e.g., FIG. 39).

In some embodiments a linker is a long linker. In some such embodiments, a long linker has approximately 7, 8, 9, 10, 11, 12, 13 or more amino acid residues. For example, in some embodiments, a long linker is or comprises an amino acid sequence of LRQKDAARGS (SEQ ID NO.13).

While these examples illustrate that linkers of different length can be used, they are not intended to limit the length or size of useful linkers. When using amino acid-based linkers, a linker may be of any length and an appropriate length will be known to those of skill in the art and dependent upon context.

In some embodiments a linker may be flexible, semi-flexible, semi-rigid, or rigid. For example, in some embodiments, a flexible linker may be or comprise an amino acid sequence comprising repeats of GGGGGS (SEQ ID NO. 69). For example, in some embodiments, an L element may be represented by a sequence of GGGGGSn, wherein n may be 1, 2, 3, 4, 5, 6, 7, 8 or more (SEQ ID NO. 244). An exemplary L element is set forth in SEQ ID NO.14, GGGGGSn, where n=6:

GGGGGSGGGGGSGGGGGSGGGGGSGGGGGSGGGGGS.

In some embodiments, a linker (e.g., a flexible linker, a semi-flexible linker, etc.) can be designed to have a more specific structure which will be well-within the ability of one of skill in the art.

In some embodiments linkers can be selected and/or designed based on domains occurring in proteins found in nature. In some embodiments linkers can be selected or designed to have a certain geometry that provides a specific orientation or spacing between a D-domain and an R-domain.

In some such embodiments, when a D element is located at a 5′ end of encoding nucleotides, and the DLR molecule comprises an L element, its L element is located at or adjacent to a 3′ end of such a D-element encoding sequence. In some embodiments, when a D element is located at a 3′ end of encoding nucleotides and the DLR molecule comprises an L element, its L element is located or adjacent to a 5′ end of a D element.

### “R” Elements

In some embodiments, agents of the present disclosure (e.g., DLR molecules comprise a D element and an R element. In some embodiments, an R element binds to a nucleic acid strand opposite to and/or complementary to a nucleic acid strand to which a D element is bound. In some such embodiments, a D domain binds to a polynucleotide (e.g., DNA) in a sequence specific manner, and an R element is capable of binding to a different molecule, for example, the opposite strand of DNA relative to where the D element is bound. In some embodiments, an R-element binds to a polynucleotide (e.g., DNA, e.g., RNA) molecule in a non-sequence-specific manner. In some embodiments, an R element binds to a polynucleotide (e.g., DNA, e.g., RNA) in a sequence-specific manner.

The present disclosure provides the insight that gene editing may be accomplished without reliance on nuclease activity to introduce breaks into one or more polynucleotide strands to be edited. The present disclosure contemplates that in some embodiments other designs of R elements are also possible, providing that such designs provide for sufficient DNA binding affinity to, e.g., stall or slow a process (e.g., replication process, transcription process, etc.) and that they have little to no inherent nuclease activity.

Accordingly, the present disclosure provides the surprising finding that gene editing may be successfully and consistently accomplished without relying on or using inherent nuclease activity to catalyze or facilitate gene editing.

In some embodiments, an R element binds to a major or minor groove. In some such embodiments, D and R elements are each bound to individual strands, but each strand is bound to the other either further upstream or downstream from where the D and R elements are bound (see, e.g., FIGS. 8A-8C).

### Sequence Specific DNA Binding R-Elements

In some embodiments an R element can also be designed to be a polynucleotide (e.g., DNA)-sequence specific binding domain. That is, for example, in some embodiments, an R element may be or comprise a zinc finger array. In some embodiments, an R element can be designed to be a 6-zinc finger array, designed to recognize the opposite strand of DNA (relative to a D element) with sequence 5′-GTGGAGCTGGACGGGGAC-3′ (SEQ ID NO.6). In some embodiments different zinc finger arrays with other DNA recognition sequences may be used as an R element. Exemplary amino acid sequences of zinc-finger arrays are provided (shown in N—C terminal orientation), and listed in Table 1.

In some embodiments, an exemplary sequence for an R-element is or comprises

(SEQ ID NO.: 86)
MAERPFQCRICMRNFSDRSNLTRHIRTHTGEKPFACDICGRKFAR
SDHLTRHTKIHTGSQKPFQCRICMRNFSDRSNLTRHIRTHTGEKP
FACDICGRKFARSDSLSEHTKIHTGSQKPFQCRICMRNFSRSSNL
TRHIRTHTGEKPFACDICGRKFARSDSLTRHTKIH

or a portion thereof.

In some embodiments other types of sequence specific polynucleotide (e.g., DNA) binding domains that will be known to those of skill in the art may be used as an R element.

### Non-Sequence Specific DNA-Binding R Elements

### Crystal Structure and Molecular Insights of Binding Nature

Crystal structures of proteins, nucleic acids and proteins bound to nucleic acids have greatly increased information and understanding of various interactions that can be involved in protein-DNA interaction. In some embodiments, interactions can be sequence specific. In some embodiments, interactions are largely non-sequence specific (e.g., interactions with a sugar-phosphate backbone (of, e.g., a target molecule, e.g., a target DNA strand, etc.); hydrophobic interactions involving a minor or major groove of a given DNA molecule, etc.). (Bogdanove, et al, 2018, Nucleic Acids Res 46 4845-4871; Rohs, et al, 2010, Annu Rev Biochem 79 233-269, each of which is herein incorporated by reference in its entirety).

### 3 Anti-Parallel Beta-Sheet Plus 2 Loop Structure

A number of structures and/or folds exist in nature as part of larger macromolecules that can bind in a non-sequence specific manner to DNA. One such macromolecular orientation can be observed in PD-(D/E)XK nuclease folds. A number of variants of this archetypical structure exist in nature and for some their crystal structure elucidation has given insights into aspects of their binding mode. Thus, in some embodiments, interactions may occur in a non-sequence specific manner. FokI nuclease domains can act in a sequence independent manner (Steczkiewicz, et al., 2012, Nucleic Acids Res 40 7016-7045, which is herein incorporated by reference in its entirety). For example, it is known in the art that crystal structure elements of FokI reveal active site residues oriented around a phosphodiester bond in a DNA backbone, while a loop structure interacts with DNA major groove atoms that are in close proximity. Accordingly, in some embodiments, interactions (e.g., DNA interactions) are not dependent presence of a specific sequence. For example, in some embodiments an R-domain can be designed using features from a core fold found in PD-(D/E)XK nucleases, wherein X is any amino acid. In some embodiments, such a fold can bind to a DNA phosphate backbone and/or to a major or minor groove of DNA in a non-sequence specific manner. In some such embodiments, any element that may have or comprise nuclease activity is modified to change a sequence of one or more active sites and reduce or eliminate any such activity. For example, in some embodiments, the first aspartic acid (“D”) residue in PD-(D/E)XK can be replaced with “A” or “N” residues. In some embodiments, residue (D/E) in a PD-(D/E)XK can be replaced with Q, N, S, T, A, V, L, I, H, R, K, or M residues.

Sequence alignment of a number of PD-(D/E)XK family members reveals that multiple members have a common core of three antiparallel beta-sheets connected by two loops (see, e.g., FIG. 39). Antiparallel beta-sheets are known, in general, to have high thermo-dynamical stability.

In some embodiments, as illustrated herein, based on amino acid sequence alignment of FokI and BtsI, a new hybrid core is designed. In some embodiments, a small structure (e.g., relative to other constructs known to those in the art and typically used in gene-editing contexts such as FokI, Cas9 and meganucleases, etc.) is designed, essentially by combining a major groove-binding loop as found in FokI with a beta sheet structure as observed in BtsI. In some such embodiments, for example, loop 2 from BtsI is selected, since it only contains 2 amino acids versus 6 amino acids in FokI. In some embodiments, based on certain biochemical principles replacing an “ND” loop structure with an “NF” will create a more thermodynamically advantageous looping structure. As will be appreciated by those of skill in the art, the PD-(D/E)xK fold exemplified herein is at least one order of magnitude smaller than other traditional constructs used in other types of gene editing. The present disclosure provides the insight that making use of smaller structures also facilitates delivery of, e.g., certain viral vectors for which other constructs would exceed capacity or “upper payload limit” such as, e.g., AAV (as compared to other viral vectors with larger packaging capacity such as, e.g., adenovirus, lentivirus, herpesvirus, etc.)

In some embodiments, an optional linker connects D and R elements. By way of non-limiting example, in some embodiments, a D element is or comprises a zinc finger array in this example (see, e.g., FIG. 39). In some embodiments, a LRGS linker (SEQ ID NO. 1) is connected to an amino acid sequence “NSGDP” (SEQ ID NO. 243) that precedes beta sheet 1 (see, e.g., FIG. 39). In some embodiments, molecular model building is used to design one or more elements as provided herein.

In some embodiments, the present disclosure provides a situation in which a core of a PD-(D/E)XK fold is stable enough and catalytic residues are mutated, such that no nuclease activity (nuclease and/or nickase) is present. In some such embodiments these structures are used as a basis for designing and/or selecting functional R elements. In some embodiments, these structures are able to bind to a polynucleotide (e.g., a DNA) backbone and their loop structures can orient such domains versus a major or minor DNA groove. For example, crystal structures and molecular modeling show orientation of core PD-(D/E)xK nuclease folds and indicate that the anti-parallel beta-sheets can (i) orient perpendicular to a DNA phosphate backbone and (ii) orient the active site towards a phosphodiester bond in that same DNA molecule. Accordingly, in some embodiments, a loop connecting two anti-parallel beta-sheets can interact with the major groove of a given DNA molecule, orienting an R element such that it binds to the DNA strand opposing a DNA strand (i.e., of the same DNA molecule) to which a D element (e.g., a zinc finger-based D element) is bound.

In some such embodiments, a nuclease fold will not have significant phosphodiesterase activity and thus, as described herein, can act as an R element.

In some such embodiments, a structure (e.g., three-beta sheet, two-loop structure) does allow binding by a DLR molecule in which a D element is or comprises a zinc finger array that binds in a sequence-specific manner to one strand of a polynucleotide, e.g., a DNA double helix, while a “loop 2” structure and linker can cause an R element to orient in such a way that it can bind to a phosphate backbone of an opposite strand of the same DNA double helix.

In some embodiments, potential active site residues that may be involved in DNA cleavage activity are mutated in order to inactivate, or greatly reduce, potential nuclease enzymatic activity. For example, in some embodiments, active site residues mutations are generated and labeled pb1 through pb12 (SEQ ID NO.34-44), and pb16 and pb17 (SEQ ID NO.45-46) (FIG. 39). The present disclosure contemplates that, in some embodiments, other amino acid substitutions and their equivalents in similar structures can be included in R elements.

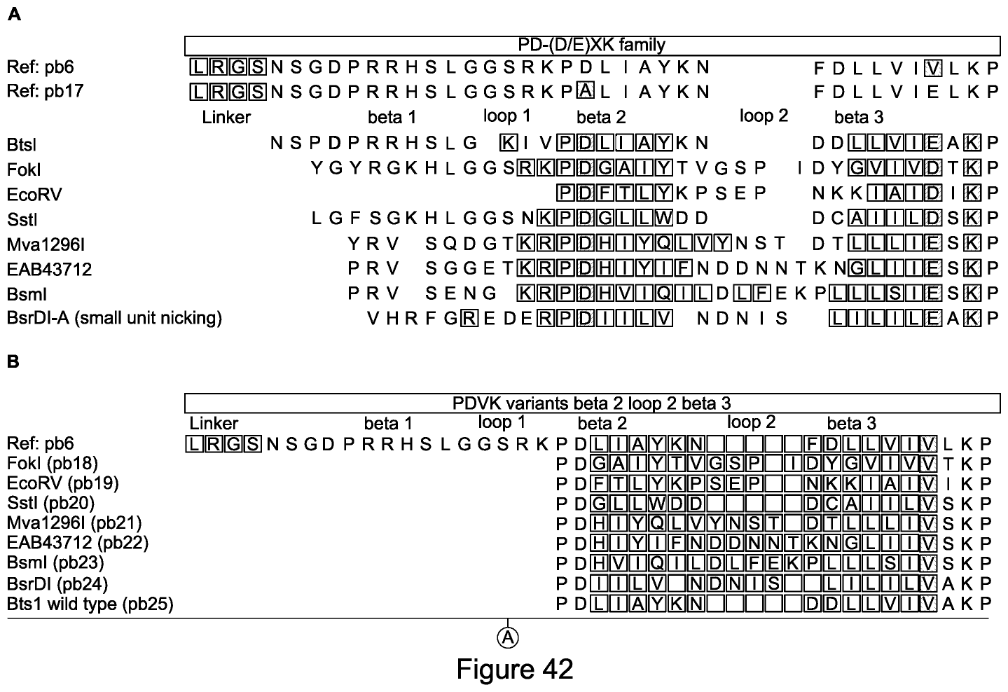

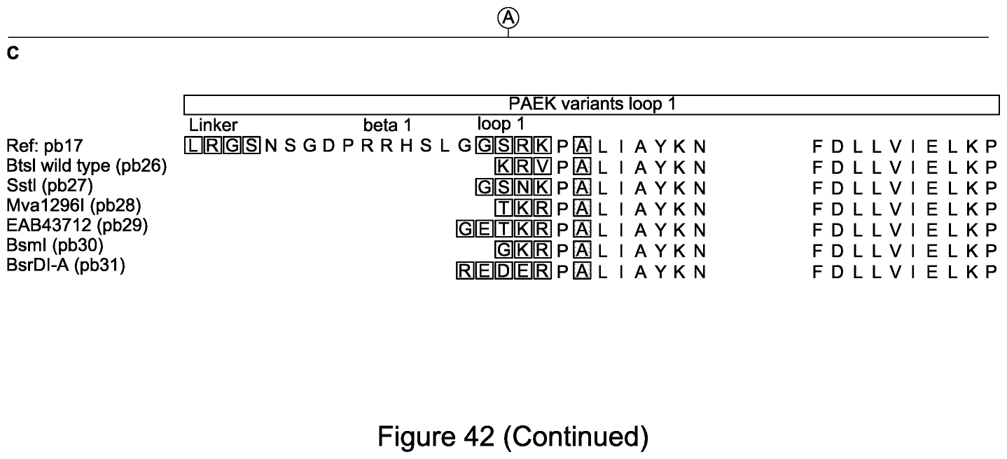

In some embodiments of the present disclosure R element design is modular. For example, as illustrated in FIG. 42, constructs are made in which a beta sheet 2-loop 2-beta sheet 3 sequence is replaced by an equivalent sequence from FokI (pb18, SEQ ID NO.47), EcoRV (pb19, SEQ ID NO.48), SstI (pb20, SEQ ID NO.49), MvaI296 (pb21, SEQ ID NO.50), EAB43712 (pb22, SEQ ID NO.51), BsmI (pb23 SEQ ID NO.52), BsrD1 (pb24, SEQ ID NO.53) respectively BtsI (pb25, SEQ ID NO.54).

In some embodiments a loop 1 structure is essentially exchangeable for equivalent structures, as illustrated by the replacement of loop 1 of construct pb17 by a similar loop 1 from BtsI (pb26, SEQ ID NO.55), SstI (pb27, SEQ ID NO.56), Mva1296 (pb28, SEQ ID NO.57) EAB43712 (pb29, SEQ ID NO.58), BsmI (pb30, SEQ ID NO.59) respectively BsrD1-A (pb31, SEQ ID NO.60).

In some embodiments other types of non-sequence specific polynucleotide recognition domains that will be known to those of skill in the art may be used as an R element or portion thereof.

### Modularity of Design of DLR

Among other things, the present disclosure provides technologies (e.g., systems, methods, compositions, etc.) such that various elements of a DLR molecule can be modular in design. For example, in some embodiments as provided herein, a D element may be or comprise a zinc finger array, a dCas9, etc. As will be apparent by those reading this disclosure, such modularity provides for a versatile and effective gene editing system, wherein, among other things and in contrast to a majority of available gene editing systems, DLR-based technologies as described herein do not depend on creation of double-or single strand DNA breaks to induce gene conversion.

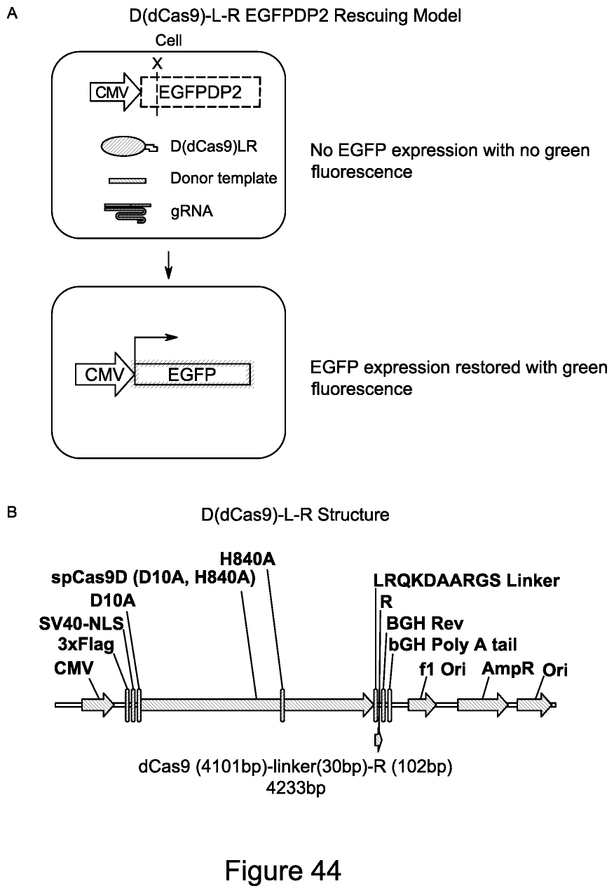

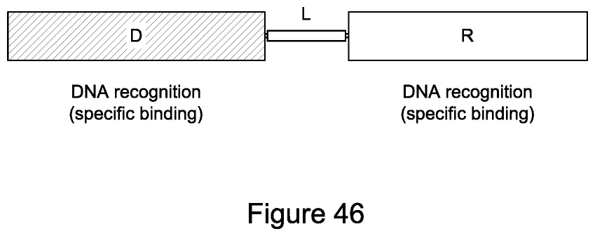

For example, in some embodiments, a DLR molecule is designed with a dCas9 protein as a D element (see, e.g., Example 7). For example, in some embodiments, different types of D elements can be used. In some embodiments other types of D elements in a given DLR containing system can be functional, assuming that they provide sequence specific nucleotide (e.g., DNA) binding. For example, in some embodiments, a D element may be or comprise a catalytically inactive Cas9 domain (rather than, e.g., a zinc finger array; see, e.g., FIG. 44). In some embodiments, modularity of DLR molecules is further provided in that an R element may be or comprise a zinc finger array (see, e.g., Example 8). In some embodiments, a DLR molecule may be or comprise a zinc finger array in each of a D and R element on a given DLR molecule (see, e.g., FIG. 46 which shows a DLR molecule comprising two DNA sequence specific binding elements (at N-terminal and C-terminal), coupled by a linker). Accordingly, in some embodiments, creation and functionality of a DLR molecule comprising zinc finger arrays in both D and R elements further illustrates that technologies of the present disclosure do not require nor depend upon nuclease or nickase activity of any particular element.

In some embodiments, an R element is modular (see, e.g., Example 6). In some aspects, successful gene conversion, using a zinc finger array as sequence specific R element, is a clear indication of versatility of DLR containing gene editing systems. In some such embodiments, the modularity of DLR molecules provides an additional advantage to gene editing beyond those advantages already conferred via no requirement for nucleotide (e.g., DNA breakage) in order to achieve a genetic modification.

### Other Modification Agents

### Sequence Modification Polynucleotides

Technologies of the present disclosure make use of sequence modification polynucleotides (e.g., donor templates, e.g., correction templates) that contain a desired genetic modification relative to a sequence of a target site. In some embodiments sequence modification polynucleotide is a donor template. In some embodiments, a sequence modification polynucleotide is a correction template. In some embodiments, a sequence modification polynucleotide can be in the form of a single stranded DNA polynucleotide. In some such embodiments, lengths of single stranded DNA oligonucleotide can range from short (e.g., at least about 12 nucleotides) to long (e.g., up to multiple kilobases). In some embodiments, a sequence modification polynucleotide can be a double stranded DNA molecule. In some such embodiments, lengths of double stranded DNA molecules can range from short (e.g., at least about 12 nucleotides) to long (e.g., multiple kilobases). In some embodiments, a double-stranded DNA molecule may be in the form of (an) artificial chromosome(s) or portion thereof. In some embodiments, a sequence modification polynucleotide can be a plasmid, viral particle and/or viral polynucleotide. In some embodiments, a sequence modification polynucleotide can comprise chemically modified nucleobases.

In some embodiments various approaches may be used to create a molecule that can act as a sequence modification polynucleotide (e.g., donor template, e.g., correction template), for example, such as by creation of a temporary single-stranded DNA structure by reverse transcription or, for example, in situations that could trigger sister-chromatid exchange. In some such embodiments, technologies provided by the present disclosure could be used for DNA modification.

In some embodiments, a sequence modification polynucleotide is a donor template. In general, a donor template is any polynucleotide sequence having sufficient complementarity with a target site to hybridize with such a target site and result in gene conversion at such a target site. In some embodiments, the present disclosure further provides for inclusion of a sequence modification polynucleotide comprising or encoding a genetic modification or modifications, that, when constitutively integrated at target site in a genome, has a therapeutic effect. For example, in some embodiments, administration of a sequence modification polynucleotide into a host cell, in combination with a DLR molecule, results in a genetic modification.

In some such embodiments, a sequence modification polynucleotide may range from 20-nucleotide to 250-nucleotide in length, or more in a single-stranded formation (e.g., a single stranded DNA formation). In some embodiments, degree of complementarity between a sequence modification polynucleotide and its corresponding target site, when optimally aligned using a suitable alignment algorithm, is about or more than about 50%, 60%, 75%, 80%, 85%, 90%, 95%, 97.5%, 99%, or more. For example, in some embodiments, a sequence modification polynucleotide may differ by only one or two bases relative to a target site. However, in some embodiments as will be understood based on context, a sequence modification polynucleotide may differ by many bases relative to a target site, for instance, in cases of genome engineering that may introduce new sites and/or structures (e.g., visualizable or trackable tags, cre-lox recombination sites, creation of indels, etc.). In some such embodiments, therefore, a portion of a sequence modification polynucleotide will have a high degree of complementarity with a given target site at one or more particular portions of the sequence modification polynucleotide (e.g., homology arms), but will differ more substantially in other areas (e.g., sites being inserted, etc.) In some embodiments, optimal alignment may be determined by using of any suitable algorithm for aligning sequences, a non-limiting example of which includes Vector NTI (Life Technologies, Waltham, MA).

### Other Agents

In some embodiments, one or more additional agents may be used in combination with one or more polymeric modification agents and/or one or more sequence modification polynucleotides. For example, in some embodiments, where a DLR molecule comprises a D element that is or comprises dCas9, a guide RNA molecule may be used to target the polymeric modification agent (via the D-element) to a particular location. In some such embodiments, in the presence of a guide RNA, a D element that is or comprises dCas9 can thus operate in a functionally similar manner as zinc-finger based D-element.

### Enhancing or Inhibiting Agents

Enhancing or inhibiting agents each refer to impact of an agent on a given activity. For example, as described herein, an RNAi technology may be an inhibiting agent if it inhibits a particular process, or it may function as an enhancing agent if it impacts a process that itself was inhibitory. In some embodiments, an enhancing agent or inhibiting agent does not itself contact a polynucleotide (e.g., DNA) being modified by a polymeric modification agent.

In some embodiments an enhancing agent or an inhibiting agent can increase or decrease levels of certain factors (e.g., replication factors, transcription factors, etc.) in a cell. For example, as will be known to those of skill in the art, in some embodiments replication factors may be or comprise one or more cellular factors (e.g., proteins, etc.) involved in various aspects of cell and DNA replication, including cell cycle regulation, DNA synthesis, DNA repair, DNA recombination and/or chromosome organization.

In some embodiments, an enhancing agent or an inhibiting agent may increase or decrease one or more transcription factors that themselves are involved in expression or regulation of genes encoding replication factors.

In some embodiments, an enhancing or inhibiting agent is an RNAi agent. RNAi refers to a biological process in which RNA molecules inhibit gene expression or translation, by neutralizing and/or reducing the cellular levels of targeted mRNA molecules. In some embodiments, RNAi is achieved using an shRNA or an siRNA molecule. For example, in some embodiments, an siRNA is used to reduce amount of genetic translational product (e.g., from RNA, e.g., mRNA, etc.). In some embodiments, RNAi is achieved using a gRNA. In some embodiments, RNAi is achieved using an oligonucleotide. In some embodiments, RNAi is achieved using an miRNA. RNA inhibition may be achieved using one or more molecules or techniques as described herein or by other methods that will be known to those of skill in the art and understood dependent on context (e.g., species, genome, system, target, etc.) In some embodiments, RNA inhibition may function as an enhancing agent.

Whether an agent is enhancing or inhibiting will be understood by those of skill in the art, depending upon context.

In some such embodiments, such other molecules impact gene conversion and/or genomic engineering. In some embodiments, cellular levels of key components (e.g., cellular replication components can be reduced or elevated by making use of certain inhibitory approaches (e.g., RNAi technologies). In some embodiments, cellular levels of key components can be reduced or elevated by making use of technologies that reduce levels of those key components in a target cell. In some embodiments, cellular levels of key components (e.g., DNA replication components, transcription components, translation components, etc.) can be reduced or elevated by making use of technologies that increase levels of those key components in a target cell.

In some embodiments, cellular levels of key components can be reduced or elevated using one or more enhancing and/or inhibiting agents, including other factors associated with DNA modification and repair, such as helicases, ligases, recombinases, repair scaffold proteins, single strand DNA binding proteins, mismatch repair proteins or any other protein that can be associated with DNA modification processes.

### Other or Additional Agents

In some embodiments, one or more additional agents may be used in conjunction with any technology described herein. For example, in some embodiments, an agent induced polynucleotide production or replication. For instance, in some embodiments, an agent induced DNA replication.

In some embodiments, an agent induced one or more breaks between one or more bases, e.g., between two nucleotides. For example, in some embodiments, an agent induces DNA breakage.

Methods Using RITDM or Transcriptional Modification for Gene Editing and/or Genomic Engineering

Among other things, the present disclosure provides methods and compositions for carrying out targeted genetic conversions (i.e., gene editing, gene conversion and/or gene targeting) or targeted gene modifications such as, e.g., suppression of transcription. The present disclosure provides technologies that, in contrast to previously disclosed methods for gene targeting, are efficient and do not depend on introducing polynucleotide (e.g., DNA) breaks into molecules comprising target sites. The present disclosure provides the insight that such technologies reduce risks of creation of unwanted indels on a target site or mutations at off-target sites. In some embodiments any segment of nucleic acid in a genome of a cell or organism can be targeted in accordance with technologies (e.g., methods) of the present disclosure.

### Methods of Making

In some embodiments, compositions, agents or systems of the present disclosure are prepared by any methods known to one of skill in the art. In some such embodiments, such preparations are formulated for delivery into a subject.

In some embodiments, compositions are prepared using any standard synthesis and/or purification system that will be known to one of skill in the art. For example, in some embodiments as described herein, one or more methods may include techniques such as de novo gene synthesis, DNA fragment assembly, PCR, mutagenesis, Gibson assembly, molecular cloning, standard single-stranded DNA synthesis, PCR, molecular cloning, digestion by restriction enzymes, small RNA molecule synthesis, cloning into plasmids with U6 promoter for RNA transcription, etc.

### Methods of Characterization

In some such embodiments, technologies of the present disclosure including a RITDM system including one or more of an agent (e.g., a blocking agent, e.g., a DLR molecule) and/or sequence modification polynucleotide and, as will be understood by one of skill in the art given context, optionally one or more additional agents such as a guide RNA or a transcriptional modification system comprising at least one agent (e.g., a polymeric modification agent, e.g., a DLR molecule comprising at least one, two, or three R elements) may be tested and/or characterized by one or more assays. For instance, by way of non-limiting example, in some embodiments, an agent (e.g., blocking agent) of the present disclosure is tested as described in Example 1 or Example 16.

In some embodiments gene conversions can be demonstrated using reporter constructs as illustrated in Example 1 such as by using a green fluorescent protein reporter construct that allows for detection of gene conversion by fluorescence detection. By way of non-limiting example, the present disclosures contemplate that in some embodiments other types of reporter constructs can be used, such as, but not limited to reporters based on fluorescent detection, bioluminescence detection, the usage of antibiotics markers, markers that make use of antibody detection and/or use of a phenotypical feature.

In some embodiments, genomic engineering, can be demonstrated using RITDM-based validation and then gene repression assays as illustrated in Example 16, which allows for confirmation of targeting and confirmation of reduction in gene transcription.

In some embodiments, the present disclosure provides an unbiased, genome-wide and highly sensitive method for detecting off-target mutations and with ability to simultaneously validate on-target gene conversion, which gene conversion may be induced by various methods of gene editing. Thus, in some embodiments, a RITDM system in accordance with the present disclosure provides comprehensive unbiased method for assessing gene editing efficiency on a genome-wide scale in cells, e.g., mammalian cells.

In some embodiments, the present disclosure provides a programmed genomic engineering method, which may achieve gene modification through, for example, suppression of polynucleotide processing (e.g., transcription). Thus, in some embodiments, a transcriptional system in accordance with the present disclosure provides a specific method for targeted programmed gene regulation in cells, e.g., mammalian cells.

In some embodiments, methods in accordance with the present disclosure (e.g., RITDM, e.g., transcriptional modification such as transcriptional suppression, with components and targets validated by RITDM) can be utilized in cell types in which a distinguishable sequence modification polynucleotide (e.g., donor template) can be efficiently analyzed if it has integrated into a targeted genome. Accordingly, in some embodiments, the present disclosure provides methods for evaluation of gene editing effects, e.g., on-target correction and off-targets mutations. In some embodiments, the present disclosure provides method for evaluation of gene regulation, e.g., suppression of gene transcription.

In some embodiments, the present disclosure provides methods applicable for evaluating editing effects as compared to other gene editing technologies including, but not limited to, engineered nucleases and nickases.

In some embodiments, analysis and/or identification of cells containing a desired genetic modification (e.g., gene conversion) may be performed in a single cell, or in a population of cells (e.g., a batch of cells, e.g., several batches or pooled populations of cells, etc.).

In some embodiments, analysis and/or identification of cells containing a desired genetic modification may be performed in (a) specific clone(s).

In some embodiments, analysis and/or identification of cells containing a desired genetic modification may be performed using a digital PCR method.

In some embodiments, analysis and/or identification of cells containing a desired genetic modification may be performed using a PCR method. In some embodiments, analysis and/or identification of cells containing a desired genetic modification may be performed using a Sanger Sequencing method. In some embodiments, analysis and/or identification of cells containing a desired genetic modification (e.g., gene conversion, e.g., transcript suppression, etc.) may be performed using a Next Generation Sequencing method. In some embodiments, analysis and/or identification of cells containing a desired genetic modification may be performed using any appropriate method to determine if one or more changes in one or more nucleotides has occurred. In some such embodiments, the present disclosure provides various methods of characterization, as described herein.

In some embodiments, analysis and/or identification of cells containing a desired genetic modification may be performed using an assay based on functionality.

In some embodiments, analysis and/or identification of cells containing a desired genetic modification may be performed using an assay based on phenotype.

In some embodiments, analysis and/or identification of cells containing a desired genetic modification (e.g., gene conversion, e.g., transcript suppression, etc.) may be performed using features of sequence modification polynucleotides (e.g., correction polynucleotides) or other components that allow identification and potentially selection for corrected cells. This may be done for example by making use of sequence modification polynucleotides (e.g., correction polynucleotides) that contain a dye or chromophore or a chemical modification (e.g., biotin) that allows for detection.

In some such embodiments, prior to implementation of programmed gene regulation, genomic targeting capacity of DLR molecules may be tested via a RITDM system. In each test, components comprise a DLR molecule and sequence modification polynucleotide. Detection of genetic conversion at a target gene is used to validate targeting capacity and specificity of a specific DLR molecule design, which, if successful, will then be used to perform targeted gene regulation. In some embodiments, an agent (e.g., blocking agent) of this present disclosure is tested as described in Example 16. In some embodiments, DLR molecules can be introduced into cells in forms of, but not limit to, DNA fragments, DNA plasmids, RNA with or without modification, and/or proteins.

In some embodiments, methods in accordance with the present disclosure can be utilized in cell types in which a targeted gene is actively transcribed into mRNA. Accordingly, in some embodiments, the present disclosure provides methods for suppressing targeted gene transcription by introduction of a DLR molecule into cells, which may be validated by total RNA extraction and quantitation. For example, in some embodiments, total RNA is reversed transcribed into DNA, which is then used for templates for PCR reactions. These two processes are used together to perform reverse transcription-polymerase chain reaction RT-PCR, which, as is known to those of skill in the art, is a sensitive technique for mRNA detection and quantitation.

### Pharmaceutical Compositions

Pharmaceutical compositions of the present disclosure may include a DLR molecule described herein. For example, in some embodiments, pharmaceutical compositions may comprise a DLR molecule. In some embodiments a pharmaceutical composition may comprise a sequence modification polynucleotide. For example, a pharmaceutical composition of the present disclosure comprising one or more agents (e.g., a blocking agent, e.g., a DLR molecule and/or a sequence modification polynucleotide and/or a guide RNA) as described herein, may be provided in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose, or dextrans; mannitol; proteins; polypeptides or amino acids such as glycine; antioxidants; chelating agents such as EDTA or glutathione; and preservatives. In some embodiments, compositions of the present disclosure are formulated for intravenous administration. Any compositions described herein can be, e.g., a pharmaceutical composition.

In some embodiments, a composition includes a pharmaceutically acceptable carrier (e.g., phosphate buffered saline, saline, or bacteriostatic water). Upon formulation, solutions will be administered in a manner compatible with a dosage formulation and in such amount as is therapeutically effective. Formulations are easily administered in a variety of dosage forms such as injectable solutions, injectable gels, drug-release capsules, and the like.

Compositions provided herein can be, e.g., formulated to be compatible with their intended route of administration. A non-limiting example of an intended route of administration is intravenous administration. In some embodiments, administration may occur ex vivo and cells may be provided post-administration, to a subject in need thereof.

Also provided are kits including any compositions described herein. In some embodiments, a kit can include a solid composition (e.g., a lyophilized composition including at least one agent as described herein) and/or a liquid for solubilizing a lyophilized composition.

In some embodiments, a kit can include a pre-loaded syringe including any compositions described herein.

In some embodiments, a kit includes a vial comprising any of the compositions described herein (e.g., formulated as an aqueous composition, e.g., an aqueous pharmaceutical composition).

In some embodiments, a kit can include instructions for performing any methods described herein.

### Cells

In some embodiments, the present disclosure provides technologies that can be used to contact one or more cells. In some embodiments, a cell is in vitro, ex vivo, or in vivo. In some embodiments, a cell (e.g., a mammalian cell) is autologous, meaning the cell is obtained, e.g., from a subject (e.g., a mammal) and cultured ex vivo.

In some embodiments, a cell is provided from a cell line, e.g., a stable cell line (e.g., HEK293, e.g., U937, etc.) In some embodiments, a cell is provided from a primary cell culture. In some embodiments, a cell is extracted from a subject in need of treatment. In some embodiments, cells are engineered to stably express exogenous genetic products. In some embodiments, a cell may be an artificial cell. In some embodiments, a cell may be an engineered cell.

In some embodiments, a cell is a human cell, a mouse cell, a porcine cell, a rabbit cell, a dog cell, a rat cell, a sheep cell, a cat cell, a horse cell, a non-human primate cell, or an insect cell.

In some embodiments, a cell is a stem cell. In some embodiments, a cell is a progenitor or precursor cell. In some embodiments, a cell is a differentiated cell. In some embodiments, a cell is a specialized cell type (e.g., a neuron, a cardiac cell, a kidney cell, an islet cell, etc.). In some embodiments, a cell is a post-mitotic cell (e.g., neuron).

In some embodiments, a host cell is transiently or non-transiently transfected with one or more vectors comprising a sequence encoding a DLR molecule and/or a sequence modification polynucleotide. In some embodiments, a cell is transfected in a substantially similar state as it occurs or exists in a subject. In some such embodiments, such a transfection may occur in vitro, ex vivo, or in vivo. In some embodiments, a cell is derived from one or more cells taken from a subject, such as development or a stable cell line and/or a primary cell culture. A wide variety of cell lines for tissue culture are known in the art. Examples of cells lines include, but are not limited to, HEK293 and U937. Cell lines are available from a variety of sources known to those with skill in the art, for example, the American Type Culture Collection (ATCC) (Manassas, VA, USA). In some embodiments, a cell transfected with one or more components of RITDM or transcriptional repression technologies as described as herein may be used establish a new cell line comprising one or more genetic modifications (e.g., any conceivable genetic modification including but not limited to loss-of-function, gain-of-function, insertion, deletion including one or more changes to create cellular models of known diseases, e.g., Alzheimer's disease or various genotypically-characterized cancers, using, e.g., known pathological mutations, targeted gene regulation to change a level of transcription/gene expression, etc.)

As will be appreciated by those of skill in the art, in some embodiments, one or more target sites may be present in a cell that is post-mitotic (e.g., neurons); that is, a cell that is not actively replicating and, therefore, incidence of replication fork activity and lagging strand exposure may be decreased relative to a cell that is, e.g., actively dividing either in a “wild-type” (e.g., skin cell, etc.) or pathogenic (e.g., cancer cell) manner. In some such embodiments, where cells that do not generally go through a phase of DNA replication are to be edited, D-loop formation during transcription may be used as alternative mechanism by which a DLR molecule may access genetic material. For example, in some such embodiments, a DNA-RNA template may be used on which a D element of a DLR molecule binds in a sequence-specific manner to a DNA strand in a post-mitotic and the R element of that DLR molecule then binds to its complementary RNA strand. Thus, by temporarily blocking D-loop structure progression, single stranded DNA will be exposed and provide opportunities for a sequence modification polynucleotide to bind.

### Combination Therapy

In some embodiments, administration can occur in combination with other molecules. For example, in some embodiments, administration can occur in combination with an enhancing agent. In some embodiments, administration can occur in combination with an inhibiting agent.

In some embodiments, an enhancing or inhibiting agent, when administered in conjunction with (e.g., sequentially or simultaneously) a polymeric modification agent and/or a sequence modification agent, may increase or decrease frequency of recombination events in a polynucleotide (e.g., DNA) contacted with the combination of an enhancing and/or inhibiting agent and polymeric modification agent, relative to frequency of recombination in a polynucleotide contacted with the polymeric modification agent without the enhancing agent.

In some embodiments, administration of combinations may include more than one combination and may, in some embodiments, occur in stages. For example, a DLR molecule may be combined with two additional agents, one of which enhances a particular process and another which inhibits a process. In some embodiments, administration may include one or more DLR molecules administered in one or more stages or combinations. For instance, by way of non-limiting example, a first combination is administered comprising a particular DLR molecule combined with an enhancing agent and a second combination is administered following a first combination, wherein the second combination combines the same or a different DLR molecule with an inhibiting agent.

In some embodiments, any forms of combination therapy that enhances survival of cells that contain (a) desired genetic change(s) may be used.

In some embodiments, other forms of combination therapy that facilitate or provide detection of cells that contain (a) desired genetic change(s) may be used.

In some embodiments, other forms of combination therapy that facilitate or provide identification of cells that contain (a) desired genetic change(s) may be used.

### Methods of Use

Gene conversion and genome engineering can be useful for a wide variety of purposes. As a consequence, many different targets can be selected for gene conversion and/or for genome engineering. For example, in some embodiments a target chosen may be for the purpose of gene conversion or genome engineering to treat human diseases. For instance, in some embodiments, monogenic diseases can be targeted by conversion of underlying mutations to corresponding sequences found in a non-affected population. Non-limiting examples of such embodiments include correction of mutations in the HPRT gene in the case of certain forms of Lesch-Nyhan syndrome, correction of certain mutations (e.g., in one or more exons known to have a mutation resulting in a DMD phenotype, e.g., exons 44, 45, 46, 47, 51, 53, etc., e.g., exon 51) in the dystrophin gene in the case of certain forms of muscular dystrophy or, e.g., correction of certain mutations in the case of the CFTR gene in the case of certain forms of Cystic Fibrosis.

In addition to monogenic diseases, gene mutations that are associated with increased risk for certain diseases can be modified to sequences that normalize or reduce that risk. For example, the ApoE gene has several variant alleles and certain variants (i.e., E4) are associated with increased risk for developing Alzheimer's disease, whereas other variants normalize (i.e., E3 allele) or even reduce (i.e. E2 allele) the risk for Alzheimer's diseases. In some embodiments, multigenic diseases could be targeted when multiple gene targets are being addressed either simultaneously or sequentially and either with one or multiple RITDM systems.

In some embodiments, a gene may silence expression and/or function of another gene and/or protein. For instance, BCL11A is a potent regulator of fetal-to-adult hemoglobin switch after birth. Generally, a higher level of BCL11A is associated with adult hemoglobin, and in patients with sickle cell anemia or β-thalassemia, adult hemoglobin is damaged. Thus, without being bound by any particular theory and by way of non-limiting example, in some embodiments, BCL11A may “silence” fetal hemoglobin (HbF) and in some embodiments, reduction or removal of such “silencing” may increase production of HbF such that symptoms of disorders involving adult beta-hemoglobin, such as B-thalassemia and sickle cell disease may be ameliorated. Accordingly, the present disclosure contemplates that, in some embodiments, decreasing levels of BCL11A using technologies provided by the present disclosure may increase HbF levels.

In some embodiments, expression of a gene may result in signaling pathways that promote or maintain a disease state. For example, in some embodiments, PD-1 signaling in immune cells (e.g., T cells) maintain and expand a cancer phenotype. PDCD1 is an immune-inhibitory receptor expressed in activated T cells and can, in some embodiments, prevent activated T cells from killing cancer cells. In some embodiments, PDCD1 is expressed in tumors, e.g., melanoma. In some such embodiments, PDCD1 expression in tumors contributes to or causes immunotherapy resistance. Without being bound by any particular theory, in some embodiments, technologies of the present disclosure contemplate that introduction of a stop codon in the PD-1 gene (i.e., PDCD-1) will reduce or eliminate PD-1 signaling. For instance, in some embodiments, a stop codon can be introduced into PDCD1 using technologies of the present disclosure; in some such embodiments, the present disclosure contemplates that such a disruption will decrease or eliminate the impact of PDCD1 signaling and may, in some embodiments, improve or enhance impact of previously ineffective or less effective immunotherapies on cancer cells. In some embodiments, a decrease in PDCD1 signaling or expression may increase T-cell mediated responses to cancer cells; in some embodiments, such cells may become sensitive to a particular treatment after gene editing as compared to cell insensitivity prior to gene editing. In some such embodiments, such genetic modifications may reduce or eliminate cancer phenotypes and/or cellular behaviors.

In some such embodiments, expression of a gene may result in or promote or maintain a disease state, but a target or mutation may be difficult to access or “drug.” For example, in some embodiments KRAS, which is a frequent oncogenic driver in solid tumors including, but not limited to, pancreatic cancer, color cancer, non-small cell lung cancer (NSCLC), etc., is often considered “undruggable,” but targeted gene regulation can result in reduction of mutated KRAS expression levels by targeting those KRAS transcripts. While, in principle, a mutated KRAS gene can be edited to a wild type KRAS gene using RITDM, once a mutation in a KRAS gene occurs (and, e.g., tumor suppression function is lost), editing that gene is not necessarily a practical way to treat a cancer. Instead, repressing the expression of the mutant KRAS gene driving a particular cancer may be effective in treating the cancer. Decrease of KRAS transcripts may be accomplished, in some embodiments, using technologies of the present disclosure to selectively target and disrupt transcription of a mutated KRAS gene. Accordingly, in some such embodiments, decrease in pathogenic KRAS transcripts with technologies provided by the present disclosure may treat or improve a disease condition.

In some embodiments a target chosen may be for the purpose of creating models useful for the study of gene conversion or genome engineering to correct and/or ameliorate human diseases. These models can be cell-based models and/or animal models.

In some embodiments a target chosen may be for the purpose of creating models useful for the study of gene conversion or genome engineering. These models may be cell-based models and/or animal models.

In some embodiments a target chosen may be for the purpose of creating models useful for the study of biological processes. These models may be cell-based and/or animal models.

In some embodiments a target chosen may be for the purpose of creating models useful for the study of disease causing processes. These models may be cell-based and/or animal models.

In some embodiments a target chosen may be for the purpose of gene conversion or genome engineering in mammalian cell lines involved in production of useful substances or features.

In some embodiments a target chosen may be for the purpose of gene conversion or genome engineering in plant cell lines involved in production of useful substances or features.

In some embodiments a target chosen may be for the purpose of gene conversion or genome engineering in eukaryotic cell lines involved in production of useful substances or features.

In some embodiments a target chosen may be for the purpose of gene conversion or genome engineering in one or more infectious agents (e.g., bacteria, parasite, virus, etc.).

In some embodiments a target chosen may be for the purpose of gene conversion or genome engineering in bacterial cell lines involved in production of useful substances or features.

In some embodiments a target chosen may be for the purpose of gene conversion or genome engineering in prokaryotic cell lines involved in production of useful substances or features.

In some embodiments a target chosen may be for the purpose of gene conversion or genome engineering in virus sequences.

Genotyping and Design of DLR Molecules and/or Sequence Modification Polynucleotides

In some embodiments, the present disclosure provides methods of making a change in genetic material (e.g., of a subject) based on analysis of a sample. For instance, in some embodiments, a sample is obtained. In some such embodiments, a sample may be tested to determine a genotype at one or more target sites and/or to determine a sequence of one or more target sequences using any number of methods known to those of skill in the art. In some embodiments, sequence analysis information is used to design and/or aid in selection of an appropriate DLR molecule and/or sequence modification agent and/or optional guide RNA that can be used to introduce a sequence modification into genetic material of a sample or of a subject from where a sample was derived. After analysis, a DLR molecule and/or sequence modification agent and/or optional guide RNA may be introduced or administered such that it is has access to or contact with genetic material to which a modification may be made.

In some embodiments, a sample is obtained or derived from a subject. In some embodiments, a subject is a control subject. In some embodiments, a subject has one or more diseases, disorders or conditions. In some embodiments, such a disease, disorder, or condition has one or more genetic changes associated therewith. In some embodiments, a subject is determined to have one or more genetic changes (e.g., genotype) associated with a particular disease, disorder or condition.

In some embodiments, a subject does not have one or more genetic changes associated with a disease, disorder, or condition, but may have an acquired phenotype that would benefit from a modification in one or more target sites and/or sequences.

In some embodiments, a DLR molecule and/or sequence modification polynucleotide and/or optional guide RNA are administered or introduced to a subject or sample derived therefrom, in need thereof. In some embodiments, a sample is acquired. In some embodiments, after acquisition, a sample may be optionally further processed (e.g., to purify, expand, test, etc.) to determine genotype information. In some embodiments, after genotypic information is determined, one or more DLR molecules and/or sequence modification polynucleotides may be designed to modify one or more target sites and/or target sequences.

In some embodiments, a DLR molecule and/or sequence modification polynucleotide and/or guide RNA is administered or applied such that it contacts genetic material to be modified. In some embodiments, administration or application is ex vivo or in vitro. In some embodiments, administration or application is in vivo. In some embodiments, after genetic material is contacted by one or more DLR molecules and/or sequence modification polynucleotides and/or guide RNA, a change in genotype detectable. In some embodiments, a change in genotype leads to a change in phenotype. In some embodiments, a change in phenotype is a reduction in one or more symptoms or manifestations of a disease, disorder, or condition, or risk thereof.

In some embodiments, after genetic material is contacted by one or more DLR molecules and/or sequence modification polynucleotides and/or optional guide RNA, no change in genotype detectable. In some such embodiments, one or more of the genetic material, DLR molecule and/or sequence modification polynucleotides and/or optional guide RNA is a control sequence designed to demonstrate no negative impact of administration of any composition comprising one or more DLR molecules and/or sequence modification polynucleotides.

In some embodiments, a sample does not come from a subject in need of treatment. For example, in some embodiments, as sample may be or comprise an infectious agent. In some such embodiments, a subject may be suffering from or at risk of infection from such an infectious agent. Accordingly, in some embodiments, a DLR molecule and/or sequence modification polynucleotide and/or optional guide RNA may be designed to inhibit or otherwise incapacitate one or more features of an infectious agent, such that risk of infection is eliminated or ameliorated. In certain embodiments of this disclosure (a) desired genetic modifications may entail a single nucleotide change, for example, in a particular gene. In certain embodiments of this disclosure a desired genetic modification may entail multiple nucleotide changes.

In certain embodiments of this disclosure a desired genetic modification may entail other forms of DNA editing.

In certain embodiments of this disclosure the desired genetic modification may entail other forms of genomic engineering.

In some embodiments, activity of a DLR molecule results in a genetic conversion of a point mutation via use of a sequence modification polynucleotide. In some embodiments, a genetic converting activity requires a complete RITDM system including a DLR molecule and sequence modification polynucleotide. For example, if a target site comprises a T→C point mutation and is associated with a risk predisposition for a disease or a disorder, in some embodiments, a target sequence comprises a C→T point mutation, wherein such a genetic conversion from C to T results in a sequence that is not associated with a risk factor with a disease or a disorder. In some embodiments, a target sequence encodes a protein and wherein a point mutation is in a codon and results in a change in an amino acid encoded by a mutant codon as compared to a wild-type codon. In some embodiments, a disease or disorder is Alzheimer's disease.

In some embodiments, genetic modification (e.g., gene conversion) can be demonstrated at a site naturally occurring within a mammalian genome. For example, in some embodiments, codon 112 of human ApoE, which comprises a point mutation that, in some embodiments, can increase predisposition to Alzheimer's disease, can be targeted and converted a DLR molecule and a sequence modification polynucleotide (see, e.g., Example 2)

In some embodiments, genetic modification (e.g., gene conversion) can be demonstrated at a number of different sites that are naturally occurring within a mammalian genome. For example, in some embodiments, codon 158 of human ApoE can be targeted and converted using a DLR molecule and a sequence modification polynucleotide (see, e.g., Example 4).

In some embodiments, the present disclosure contemplates that any site within a genome can be modified. For example, as described above and herein, in some embodiments, a cell can harbor one or more point mutations in its genome. In some such embodiments, for example, one or more point mutations can exist, e.g., T-to-C or C-to-T. By way of non-limiting example, point mutations at codons 112 and 158 in the human ApoE gene can result in C112R and R158C amino acid mutations, respectively. In some such embodiments, changing one or more of these point mutations using a DLR molecule and sequence modification polynucleotide can change one or more nucleotides in codon 112 and/or 158, resulting in a change of an ApoE isoform from pathogenic to non-pathogenic, e.g., from more likely to develop Alzheimer's disease to less likely to develop Alzheimer's disease, e.g., based on an ApoE genotype. For example, in accordance with the present disclosure, a genetic modification can be made at ApoE codon 112 to achieve a C to T gene conversion (see, e.g., Example 5; U937 cell line) or a T to C conversion (see, e.g., Example 2). The present disclosure contemplates that in some embodiments, any number of cell lines or primary cell cultures may be used and such cells will be known and/or understood by those of skill in the art dependent upon context.

The present disclosure provides the insight that successful correction of pathogenic gene variants (such as mutations) in genes associated with one or more diseases, disorders and/or conditions provides new strategies for gene correction. In some embodiments a RITDM system can be used to correct other mutations associated with any disease, disorder and/or condition.

In some embodiments, sequence-specific and site-specific gene modification approaches comprising, e.g., a DLR molecule, a sequence modification polynucleotide and/or systems such as the RITDM system which comprises both a DLR molecule and a sequence modification polynucleotide can be used to modify genes in such a way that certain gene functions are eliminated or abolished. For example, in some embodiments, a RITDM system may be used for generation of premature stop codons (TAA, TAG, TGA) to abolish protein functions, for example, in cancers.

In some embodiments, such technologies may be used, for example, in laboratory or research settings to design new cell lines for use in, e.g., development of therapeutics or screening of disease states or, e.g., screening of compound, etc.

In some embodiments, the present disclosure provides new methods and reagents for gene conversion and genome engineering. For instance, as illustrated in Example 3 a DLR-based gene-editing system can yield important advantages such as off-target effects occurring at very low frequencies.

### DLR Designs for Programmed Gene Regulation

In some embodiments, a polymeric modification agent such as a DLR molecule of the present disclosure may comprise one or more R elements. In some such embodiments, multiple R elements (i.e., two or more) are tethered. Without being bound by any particular theory the present disclosure contemplates that two or more R elements increase non-sequence specific DNA binding capacity, for example, as in a DLR molecule according to the formula D-L-R—R, in which two R elements are linked together or D-L-R—R—R in which three R elements are linked together. In some embodiments, a given R element may have the same or different sequence than one or more additional R elements of the same DLR molecule. For instance, by way of non-limiting example, in a molecule with three R elements, each R element may have a unique sequence, each R element may share certain sequence portions of features, and/or each R element may comprise the same or substantially the same sequence as one or both of the other two R-elements.

In some embodiments, an exemplary R element for use in a DLR molecule comprising one, two, three or more R-elements comprises one or more of the following DNA sequences. By way of non-limiting example, the following sequences are derived from PD-(D/E)xKP family which comprises a 3 anti-parallel beta-sheet plus two loop structure. The sequences are displayed from 5′- to 3′-end, and followed with its corresponding amino acid sequence, displayed from N-terminal to C-terminal.

(SEQ ID NO.: 207)
5′-AATTCTGGTGATCCTCGGAGACACAGTCTGGGCGGTTCTCGT
AAACCCGATCTGATTGCCTATAAAAACTTTGATCTGCTGGTCATT
GTTCTTAAGCCT-3′.
(SEQ ID NO.: 208)
NSGDPRRHSLGGSRKPDLIAYKNFDLLVIVLKP.
(SEQ ID NO.: 209)
5′-AATTCTGGTGATCCTCGGAGACACAGTCTGGGCGGTTCTCG
TAAACCCGATGGTGCTATTTATACTGTTGGTTCTCCTATTGATTA
TGGTGTTATTGTTGTTACTAAACCT-3′.
(SEQ ID NO.: 210)
NSGDPRRHSLGGSRKPDGAIYTVGSPIDYGVIVVTKP.
(SEQ ID NO.: 211)
5′-AACTCTGGTGATCCTCGGAGACACAGTCTGGGCGGTTCTCGT
AAACCCGATATTATTCTTGTTAATGATAATATTTCTCTTATTCTT
ATTCTTGTTGCTAAACCT-3′.
(SEQ ID NO.: 212)
NSGDPRRHSLGGSRKPDIILVNDNISLILILVAKP.

In some embodiments, a “double” R element can be linked to an L element comprises a DNA sequence of 5′-AATTCTGGTGATCCTCGGAGACACAGTCTGGGCGGTTCTCGTAAACCCGATCTGATT GCCTATAAAAACTTTGATCTGCTGGTCATTGTTCTTAAGCCTAAATACTCCCAGAATT CTGGTGATCCTCGGAGACACAGTCTGGGCGGTTCTCGTAAACCCGATGGTGCTATTT ATACTGTTGGTTCTCCTATTGATTATGGTGTTATTGTTGTTACTAAACCT-3′ (SEQ ID NO. 213) and its corresponding amino acid sequence is, from N terminal to C terminal, NSGDPRRHSLGGSRKPDLIAYKNFDLL VIVLKPKYSQNSGDPRRHSLGGSRKPDGAIYTV GSPIDYGVIVVTKP (SEQ ID NO. 214). The first R element and the second R element are linked with two amino acids, “SQ.”

In some embodiments, a “triple” R element is linked to an L element comprises a DNA sequence of 5′-AATTCTGGTGATCCTCGGAGACACAGTCTGGGCGGTTCTCGTAAACCCGATCTGATT GCCTATAAAAACTTTGATCTGCTGGTCATTGTTCTTAAGCCTAAATACTCCCAGAATT CTGGTGATCCTCGGAGACACAGTCTGGGCGGTTCTCGTAAACCCGATGGTGCTATTT ATACTGTTGGTTCTCCTATTGATTATGGTGTTATTGTTGTTACTAAACCTAAGTACTC CCAGAACTCTGGTGATCCTCGGAGACACAGTCTGGGCGGTTCTCGTAAACCCGATAT TATTCTTGTTAATGATAATATTTCTCTTATTCTTATTCTTGTTGCTAAACCT-3′ (SEQ ID NO. 215), with its corresponding amino acid sequence is, from N terminal to C terminal, NSGDPRRHSLGGSRKPDLIAYKNFDLLVIVLKPKYSQNSGDPRRHSLGGSRKPDGAIYTV GSPIDYGVIVVTKPKYSQNSGDPRRHSLGGSRKPDIILVNDNISLILILVAKP (SEQ ID NO. 216). The first and second and second and third R elements are linked to each other with two amino acids, “SQ.”

### Methods of Treatment

In some embodiments, technologies of the present disclosure are used to treat subjects with or at risk of a pathogenic phenotype due to an underlying (e.g., inherited, e.g., acquired) genotype. For example, in some embodiments, a subject has a point mutation in an ApoE gene, which produces an allele that generates an isoform that is associated with a higher risk of developing Alzheimer's disease. In some embodiments, technologies of the present disclosure may be used to treat diseases, disorders or conditions that are caused by one or more mutations in at least one target sequence; for example, in some embodiments, a subject may have a mutation in, for example, a CFTR gene, which mutation causes cystic fibrosis. In some embodiments, a subject may have one or more mutations in the human dystrophin gene resulting in muscular dystrophy, e.g., Duchenne muscular dystrophy. For example, in some embodiments, one or more mutations in the dystrophin gene may result in a frame shift such that dystrophin production is reduced or eliminated. In some embodiments, technologies of the present disclosure may introduce one or more genetic modifications such that a functional reading frame is restored and some amount of dystrophin protein (either in full or truncated form) is produced.

In some embodiments, technologies of the present disclosure may be used to treat cancer. For example, in some embodiments, a cancer may be hereditary (e.g., BRCA1 gene mutation) or inherited (e.g., spontaneous mutation causing, e.g., leukemia). In some such embodiments, technologies of the present disclosure may be used to change genotypes of one or more cells comprising a cancer-associated (e.g., cancer causing) genetic sequence.

In some embodiments, technologies of the present disclosure may be used to achieve genetic modifications that result in removal of a gene regulation function. For example, in some embodiments, BCL11A may silence fetal hemoglobin (HbF). In some such embodiments, reduction or removal of such silencing may increase production of HbF such that symptoms of disorders involving adult beta-hemoglobin, such as β-thalassemia and sickle cell disease may be ameliorated. Without being bound by any particular theory, the present disclosure contemplates that, in some embodiments, decreasing levels of BCL11A using technologies provided by the present disclosure may increase HbF levels. In some embodiments technologies of the current disclosure may be used in immune-related treatments (e.g., immuno-oncology or other immune diseases, disorders or conditions). For example, in some embodiments genetic modifications may be made to one or more genes involved in immune function and/or immune regulation. In some such embodiments, technologies of the present disclosure may be used to change a genotype of one or more cells or cell types comprising an immuno-associated genetic sequence (e.g., T-cell receptor alpha, T-cell receptor beta, PD-1 (i.e., PDCD-1), PD-L1 CTLA-4, TREM2). For example, in some embodiments, the present disclosure contemplates that editing PDCD-1 by introducing a stop codon may decrease or eliminate PD-1 signaling such that, in some embodiments, cancer activities are reduced or eliminated. In some embodiments, a cancer cells, after editing, may become more responsive or may become sensitive to a treatment (as compared to, e.g., prior to editing where, in some embodiments, a cancer cell may not have been sensitive or responsive to a particular treatment).

By way of non-limiting example, for instance, in some embodiments technologies of the present disclosure may be used to support development of cellular technologies that aim to treat cancer-associated conditions or immune-dysbiosis related conditions.

In some embodiments, technologies of the present disclosure may be used to treat one or more infectious diseases, disorders or conditions. For example, in some embodiments, an infectious disease may be caused by bacteria, parasites, and/or viruses. For example, the present disclosure provides technologies that may be used, e.g., to interfere with replication and/or proliferation of a virus or bacteria.

In some embodiments, the present disclosure provides methods of determining a genotype of a subject or a sample as described herein. In some such embodiments, determining a genotype is used in diagnosing and/or treating a subject as described herein.

It will be understood by those in the art that many different changes (e.g., substitutions, deletions, additions, etc.) in any genetic material can result in or risk causing one or more pathogenic phenotypes.

In some embodiments, programmed gene regulation, as provided in accordance with the present disclosure, may be used to treat subjects with, or at risk of one or more pathogenic phenotype due to an underlying (e.g., inherited, e.g., acquired) genotype. For example, in some embodiments, a subject has mutation in a KRAS gene. In some such embodiments, a mutation in a KRAS gene results in an allele that generates a KRAS isoform that is associated with a higher risk of developing cancer. In some such embodiments, a cancer may include, but not be limited to, pancreatic cancer, colon cancer, and/or non-small cell lung cancer (NSCLC).

In some embodiments, programmed gene regulation as provided by the present disclosure may be used to treat one or more autosomal dominant genetic diseases in which a single copy of a disease-associated mutation has, will or is able to cause a disease. As provided herein, in some embodiments, a polymeric modification agent such as a sequence-specific DLR molecule is able to distinguish a mutated gene sequence from wild-type (“normal” or non-disease associated) loci and preferentially suppress expression of a mutated gene or related sequence. In some embodiments, technologies provided herein can be used to treat diseases that result from genetic mutations that are not amenable to treatment with approaches such as gene editing, including, but not limited to, autism or polycystic kidney disease.

### Administration

In some embodiments, an agent of the present disclosure is or comprises a DLR molecule in combination with a sequence modification polynucleotide that can be used to generate or induce sequence (e.g., nucleotide) conversions. In some such embodiments, methods comprise delivering one or more sequence modification polynucleotides, such as one or more vectors and/or one or more transcripts thereof, and/or one or more proteins transcribed therefrom in accordance with the present disclosure, to a host cell.

In some embodiments, the present disclosure further provides cells produced by such methods and organisms (such as animals, plants, or fungi) comprising or produced from such cells as described herein. In some embodiments, for example, a DLR molecule in combination with a sequence modification polynucleotide such as a donor template, comprise an exemplary RITDM system. In some embodiments, such an exemplary RITDM system is delivered to a cell. In some such embodiments, delivery is achieved by contacting a cell with one or more components of a RITDM system, e.g., one or more agents of the present disclosure (e.g., one or more blocking agents and/or one or more sequence modification polynucleotides). In some embodiments conventional non-viral- or viral-based gene transfer methods that are known to those of skill in the art can be used to introduce nucleic acids (e.g., one or more components of a RITDM system as described herein) into cells, e.g., mammalian cells, e.g., human cells. In some embodiments, such methods can be used to administer nucleic acid encoding components of a RITDM system to cells in culture (e.g., in vitro or ex vivo), or in a host organism (e.g., in vivo or ex vivo).

By way of non-limiting example, in some embodiments non-viral vector delivery systems include DNA plasmids, RNA (e.g., a transcript of a vector described herein), naked nucleic acid, and/or nucleic acid complexed with a delivery vehicle, such as liposome. In some embodiments, viral vector delivery systems include DNA and RNA viruses, which have either episomal or integrated genomes after delivery to the cells.

In some embodiments introduction of a DLR molecule and polynucleotide template can be performed by transfection. In some embodiments, introduction of DLR molecule and sequence modification polynucleotide can be performed by nucleofection. In some embodiments, introduction of a DLR molecule and sequence modification polynucleotide can be performed by any known or appropriate route of introduction into a target cell (e.g., a cell comprising at least one target site).

In some embodiments, a target site comprises a small deletion, insertion and/or single nucleotide polymorphism within a coding sequence of a gene. In some embodiments, a target site comprises more than one mutations, for example, a deletion and a point mutation wherein these two mutations are located adjacent to one another. In some embodiments, a deletion is associated with early termination of translation of a gene product (e.g., a protein) because of, e.g., generation of a premature stop codon and/or reading frame shift.

In some embodiments, activity of an agent (e.g., a given DLR molecule) in combination with a sequence modification polynucleotide of a RITDM-system results in genetically correcting a deletion, insertion and/or single nucleotide polymorphism to restore an appropriate reading frame and translate into a normal and functional gene product. In some embodiments, activity of a DLR molecule in combination with a sequence modification polynucleotide of a RITDM-system results in correction of two mutations simultaneously. In some embodiments “larger” insertions, deletions, gene rearrangements and/or chromosome rearrangements may be involved. For example, in some embodiments, a “larger” change may be, as described herein, in contexts of genome engineering including but not limited to insertions of visualizable or detectable tags, cre-lox components, indels, etc. In some embodiments, for example, gene conversions of one, two, or several nucleotides would not be considered “larger”. In some embodiments other forms of gene repair and/or genome engineering may be performed by using a RITDM-system.

### EQUIVALENTS

It is to be understood that while the disclosure has been described in conjunction with the detailed description thereof, the foregoing description is intended to illustrate and not limit the scope of the present disclosure, which is further defined by the scope of the appended claims. Other aspects, advantages, and modifications are within the scope of the following claims.

### EXAMPLES

The following examples are given for the purpose of illustrating various embodiments of the disclosure and are not meant to limit the present disclosure in any fashion. Changes therein and other uses which are encompassed within the spirit of the disclosure as defined by the scope of the claims will occur to those skilled in the art.

### Example 1: A DLR-Based DNA Conversion System Enables Targeted Conversion of Mutant EGFP Gene in a Genome

In order to demonstrate that a DLR molecule can be used for gene conversion, a reporter system based on an Enhanced Green Fluorescent Gene (EGFP) was created. Essentially this cell-based model allows for detection of gene conversion by activation of green fluorescence.

### Exemplary Assay 1

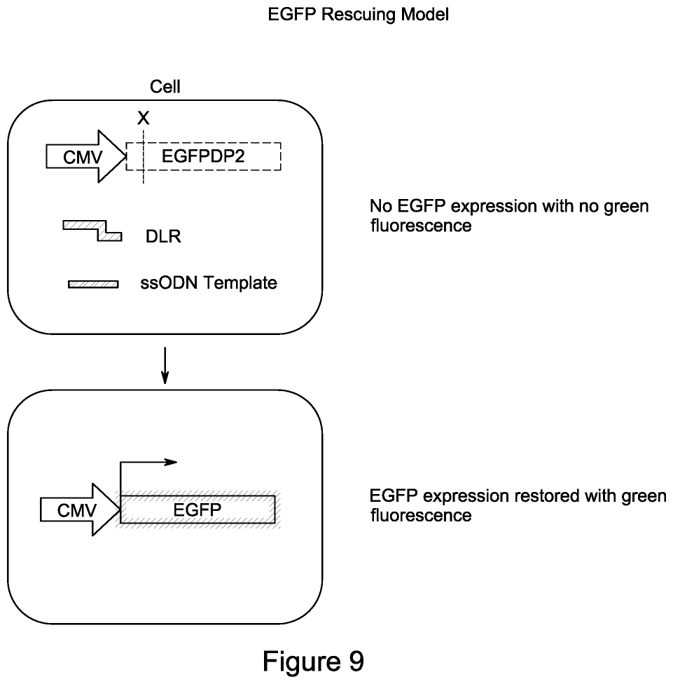

FIG. 9 shows an EGFPDP2 gene mutation repair assay principle. A reporter cell line was created, in which a mutated and inactivated EGFPDP2 gene was stably integrated into a genome under control of a CMV promoter in an HEK293 cell line. In this cell line, only a truncated EGFPDP2 was expressed, preventing green fluorescent signal from being detected above background levels. A DLR molecule was designed to target a target site close to two mutations in the EGFPDP2. A correction template was designed to convert these two mutations back to a coding in-frame EGFP sequence. Repair of the mutant EGFPDP2 using this gene conversion system and DLR molecule resulted in restoration of expression of detectable EGFP, as evidenced by detection of green signal by fluorescent microcopy and sequencing confirmation.

### Exemplary Assay II

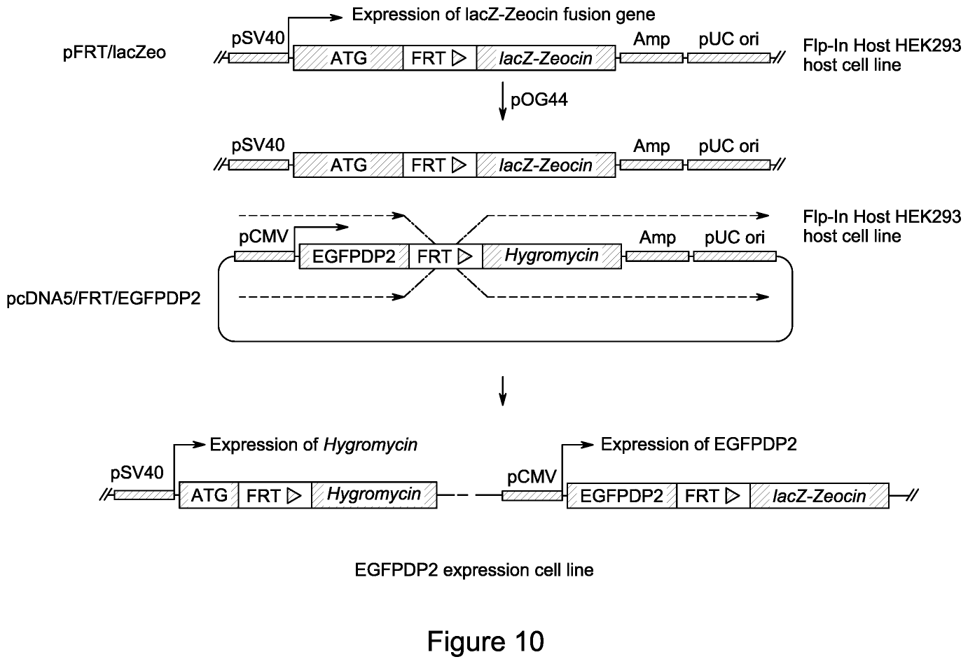

FIG. 10 shows an exemplary engineering schematic of an EGFPDP2 reporter cell line using an HEK293 FlpIN system (Life Technologies, Carlsbad, CA). Here, EGFP was integrated into the genome of HEK293 cells. To begin, a FlpIN host cell line was used. This line contains a fusion gene of LacZ-Zeocin stably inserted into its genome by a transfection of plasmid of pFRT/lacZeo (Life Technologies, Carlsbad, CA). This gene is driven by a SV40 promoter and it has an FRT site inserted after its ATG start codon, making this FlpIN host HEK293 cells resistant to zeocin containing medium. Plasmid pcDNA5/FRT/EGFPDP2 (SEQ ID NO.17) was constructed by cloning EGFPDP2 coding sequencing into plasmid vector pcDNA5/FRT with CMV promoter (Life Technologies, Carlsbad, CA). Plasmid pcDNA5/FRT/EGFPDP2 was co-transfected with plasmid pOG44 (Life Technologies, Carlsbad, CA) into this HEK293 FlpIN host cell line. pOG44 expresses a recombinase and induced recombination at the two FRT sites present in this system: one in the cellular genome and one on plasmid pcDNA5/FRT/EGFPDP2. Successful recombination was demonstrated by resistance to hygromycin. Hygromycin resistance can be conferred by an out-of0frame shift of lacZ-zeocin and simultaneous expression a hygromycin resistance gene upstream. Cells expressing the EGFPDP2 gene survived in hygromycin.

### Exemplary Assay III

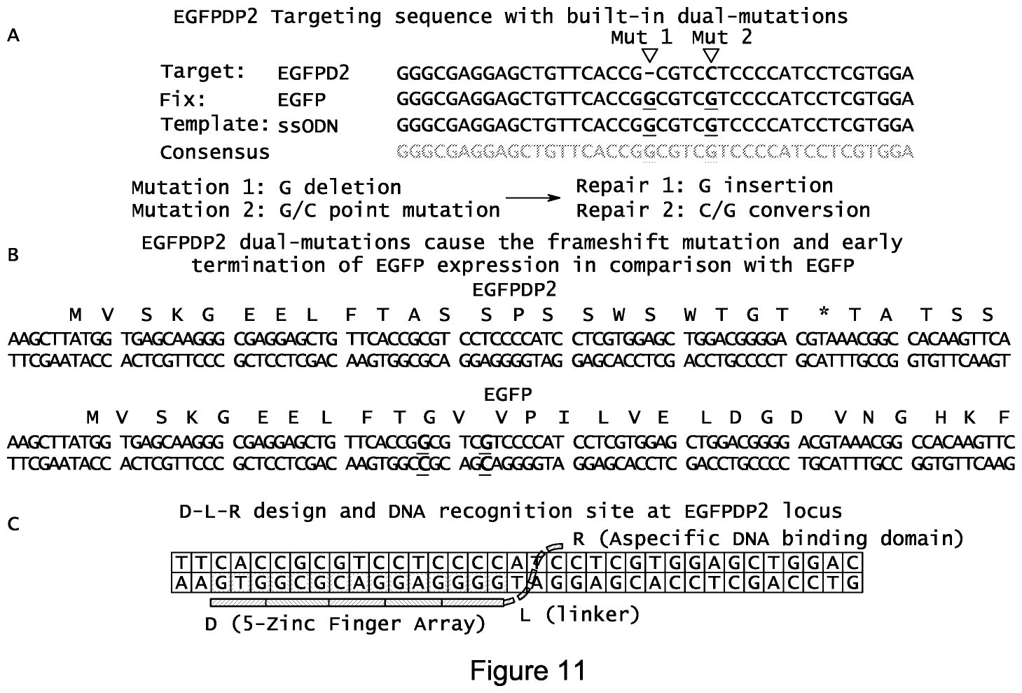

FIG. 11 illustrates molecular details of core elements of this specific gene conversion system. Panel A shows DNA sequences of EGFPDP2, ssODN template (i.e., sequence modification polynucleotide), and EGFP and two mutations at this targeting site. EGFPDP2 targeting and repairing was based on two mutations: a deletion of nucleotide G and a G→C point mutation. A donor template was designed to insert a G and convert a C to G at these two mutation sites of EGFPDP2. A successful EGFPDP2 gene repair would restore in-frame expression of EGFP. Panel B shows protein translations prior to and post gene conversion. The EGFPDP2 (SEQ ID NO.15) gene was mutated and frame-shifted resulting in an early termination due to these two mutations. That is, instead of the wild type protein (shown in SEQ ID NO 16, reading “MVSKGEELFTGVVPILVELDGDVNGHKFSVSGEGEGDATYGKLTLKFICTTGKLPVPW PTLVTTLTYGVQCFSRYPDHMKQHDFFKSAMPEGYVQERTIFFKDDGNYKTRAEVKFE GDTLVNRIELKGIDFKEDGNILGHKLEYNYNSHNVYIMADKQKNGIKVNFKIRHNIEDGS VQLADHYQQNTPIGDGPVLLPDNHYLSTQSALSKDPNEKRDHMVLLEFVTAAGITLGM DELYK*,” the frame shift results in a sequence that has stop codons introduced throughout as follows “MVSKGEELFTASSPSSWSWTGT*TATSSACPARARAMPPTAS*P*SSSAPPASCPCPGPPS*PP*PTACSASAATPTT*SSTTSSSPPCPKATSRSAPSSSRTTATTRPAPR*SSRATPW*TASS*RASTSRRTATSWGTSWSTTTTATTSISWPTSRRTASR*TSRSATTSRTAACSSPTTTSRTPPSATAPCCCPTTTT*APSPP*AKTPTRSAITWSCWSS*PPPG SLSAWTSCTS” where * represents a stop codon. Thus, the truncated version is “MVSKGEELFTASSPSSWSWTGT*” resulting in the protein of SEQ ID NO. 15) being produced. Successful genetic conversion restored functional EGFP (SEQ ID NO.16) expression, resulting in in-frame protein translation.

Panel C illustrates that this EGFPDP2 locus was targeted by this DLR construct. Plasmid pb34 (SEQ ID NO.18), as an example, encoded this specific DLR construct, which contained a 5-zinc finger array as a D element, designed to recognize a strand of DNA with sequence 5′-GGGGAGGACGCGGTG-3′ (SEQ ID NO.4). This DNA recognizing zinc finger array was extended by a linker domain (LRGS, SEQ ID NO. 1) followed by an R-element. A DNA construct encoding the DLR molecule of the present Example was cloned using HindIII and NotI sites at the 5′ to 3′ ends respectively. A mammalian expression vector pVAX1 (ThermoFisher, Waltham, MA) was used, making use of its kanamycin antibiotic resistant gene. Two variants of this construct were created: pb34 (SEQ ID NO.18) and pb35 (SEQ ID NO.71). pb34 and pb35 differ in the inactivated catalytic residues within their respective R elements. In this specific embodiment, amino acid sequence of an R element in pb34 is NSGDPRRHSLGGSRKPDLIAYKNFDLLVIVLKP (SEQ ID NO.19), while that in pb35 is NSGDPRRHSLGGSRKPALIAYKNFDLLVIELKP (SEQ ID NO.84). An encoding DNA sequence for each R element is listed in Table 1 (SEQ ID NOS.: 20 and 85). At the 5′-end of these DLR-encoding sequences, DNA encoding a FLAG-tag and NLS signals was inserted. Pb34 and pb35 cDNA coding sequences (SEQ ID NOS.: 74 and 72), as well as their corresponding amino acid sequences (SEQ ID NOS.: 75 and 73), are listed.

EGFPDP2 reporter cells were cultured in hygromycin DMEM medium supplemented with 10% Fetal Bovine Serum (FBS). Twenty-four hours prior to electroporation, cells were exposed to thymidine at a concentration of 5 mM for 18 hours. Electroporation was performed using a HEK293 transfection kit and a nucleofection instrument to transfect either pb34 or pb35 along with a 142-nucleotide single stranded ODN template (SEQ ID NO.: 70). After nucleofection, transfected cells were placed onto a plate pre-coated with 0.1% gelatin (to enhance survival and adherence). Culturing continued at 5% CO2 in a 37° C. incubator for at least 5 days. Culture medium was exchanged regularly.

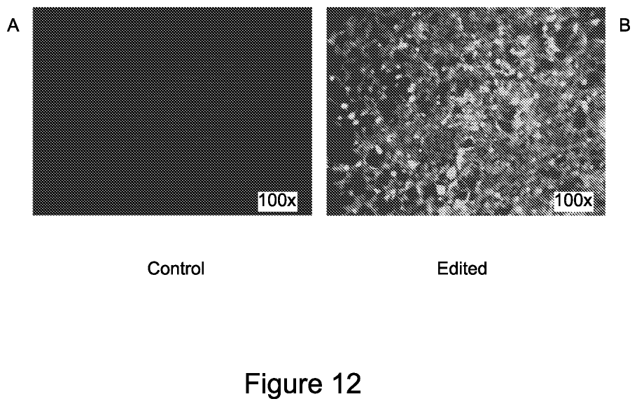

Starting at day 5 post transfection, a small number of cells turned fluorescent green, as could be observed under a fluorescent microscopy. Continuation of culture after supplying fresh culture medium yielded more green cells, some of which were growing into green fluorescent clusters. Green cells were enriched after partial trypsinization and allowed to continue culturing in a 24-well plate. Green cells were analyzed using fluorescent microscopy, as shown in FIG. 12. In panel A, cells carrying EGFPDP2 did not show signs of green florescence under these conditions as tested. After gene conversion, cells that were repaired by action of this DLR protein and donor template showed green fluorescence, as shown in panel B.

Green cells were further allowed to proliferate to more than 50% confluence. Genomic DNA was then extracted and purified by 100% ethanol precipitation. Analysis of genetic modifications was conducted using PCR analysis, Sanger sequencing as well as next-generation sequencing. PCR reactions were set up using Phusion Hi-Fi DNA polymerase (New England Biolabs, Ipswich, MA) with a primer set: 5′-CCATATATGGAGTTCCGCGTTAC-3′ (SEQ ID NO.76) and 5′-GCTTGTCGGCCATGATATAG-3′ (SEQ ID NO.: 77). PCR conditions included steps at 98° C. for 15 seconds of denaturation followed by 35 cycles of 98° C. for 10 seconds and 72° C. for 15 seconds, and 72° C. for 1-minute final extension. PCR products were cleaned by column purification and sequenced using above primers (SEQ ID NO.76 and 77).

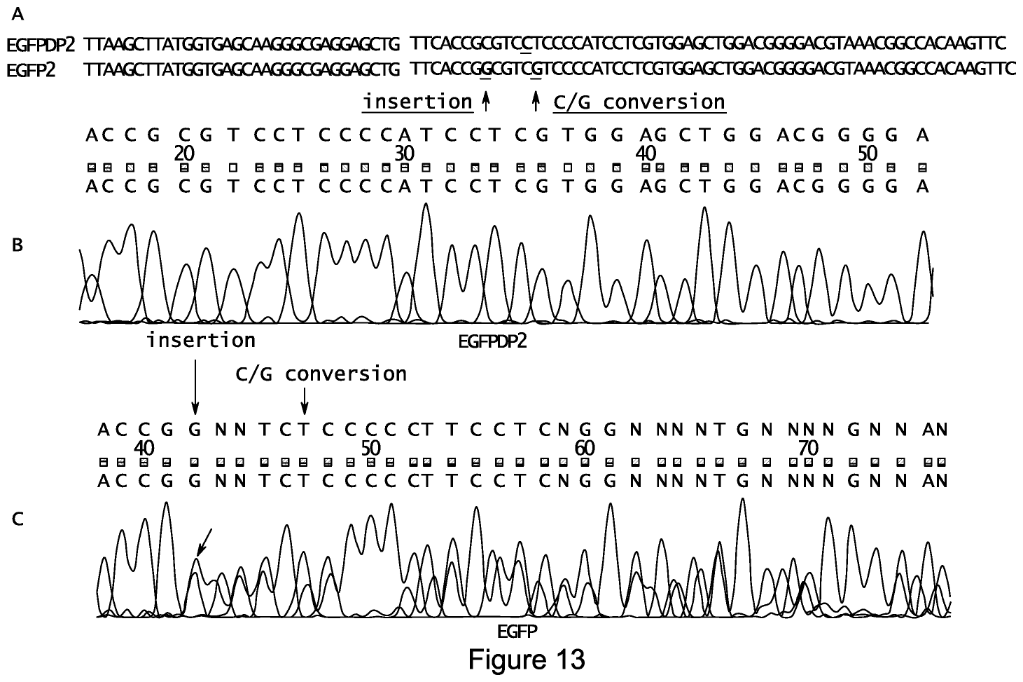

FIG. 13 shows Sanger Sequencing results used to confirm successful EGFPDP2 targeting and repairing. Panel A demonstrates a DNA sequence alignment of EGFPDP2 and EGFP (positions of 2 mutations indicated by arrows). After gene conversion, an insertion of nucleotide G shifted this EGFP DNA sequence one nucleotide to the right, and therefore downstream sequences between EGFPDP2 and EGFP were not matched to each other. An exemplary chromatogram of EGFPDP2 by Sanger Sequencing in Panel B shows one trace of nucleotide spike at each position, demonstrating homozygosity of EGFPDP2. However, as seen in Panel C, gene conversion resulted in two chromatograms overlapping each other, beginning at the indicated position of insertion. Because one allele of EGFPDP2 gene was converted into EGFP, the genotype of these cells became heterozygous. These results demonstrated that a DLR molecule in combination with a suitable correction template could be used for targeted gene conversion in mammalian cells.

To further analyze effects of this novel approach to gene conversion, next generation sequencing was performed to determine genetic conversions and background damages by undesired insertions and deletions (Indels). Genomic DNA derived from single green fluorescent clones was used, while a negative clone and untargeted EGFPDP2 were used as controls. For next generation sequencing, a 171-bp PCR amplicon from this EGFPDP2 targeting region was generated using Phusion PCR protocol similar to that used for generating material for Sanger Sequencing, using primer sets: 5′-CCAAGCTGGCTAGCGTTTA-3′ (SEQ ID NO.: 78) and 5′-GAACTTCAGGGTCAGCTTGC-3′ (SEQ ID NO.: 79), which were flanking this target site. PCR products were purified using a gel extraction kit (Thermo Fisher Scientific, Waltham, MA). Twenty-five micrograms of purified PCR products were analyzed using an “Amplicon-EZ” procedure on an Illumine 2×250 base-pair platform (GENEWIZ, South Plainfield, NJ), and Fastq files for each gene-primer pair were aligned to a custom genome file containing that gene locus using bioinformatic analysis with default parameters, which all gave similar results (GENEWIZ, South Plainfield, NJ).

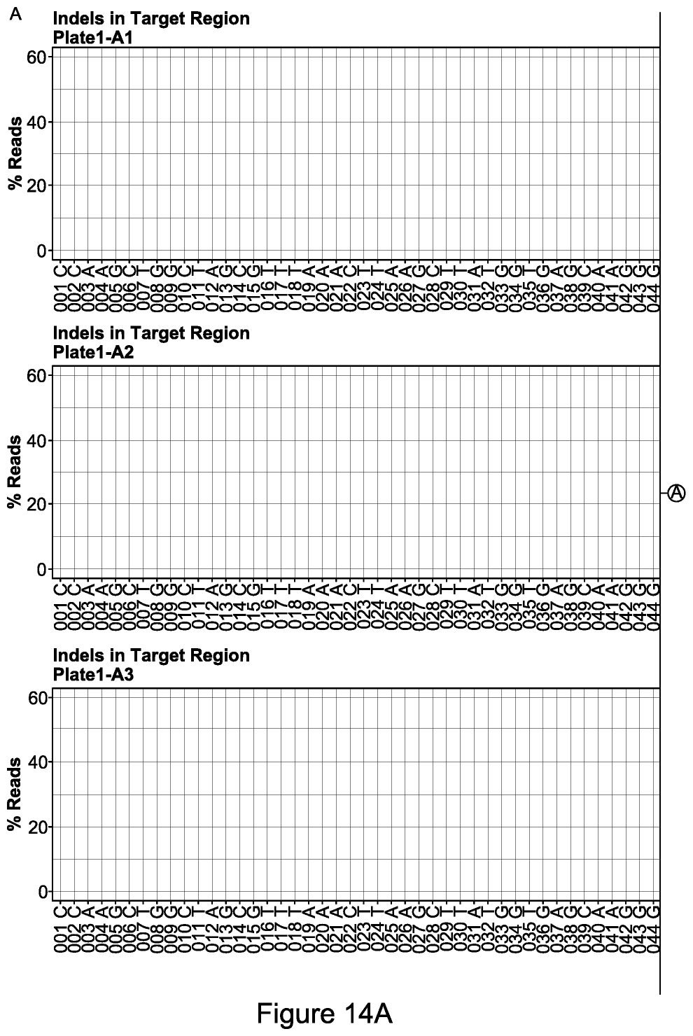

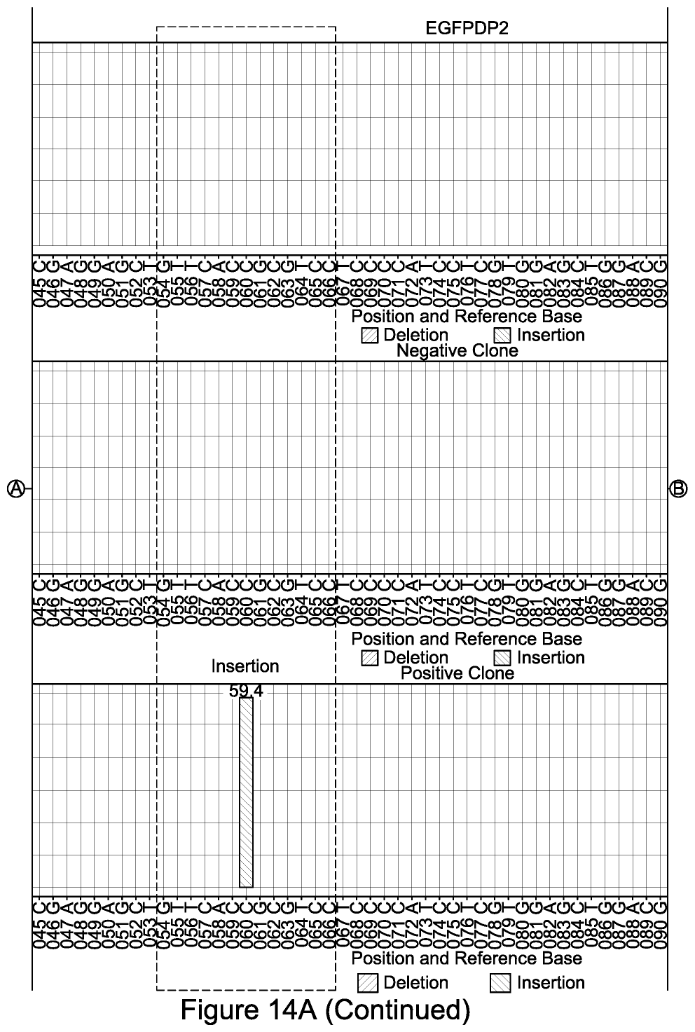

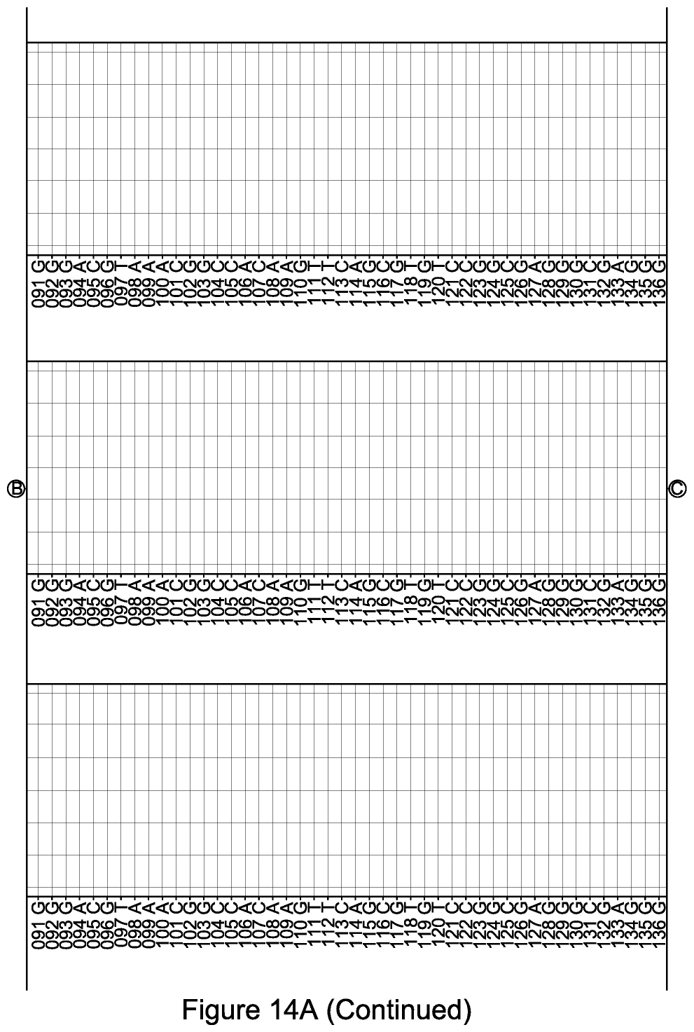

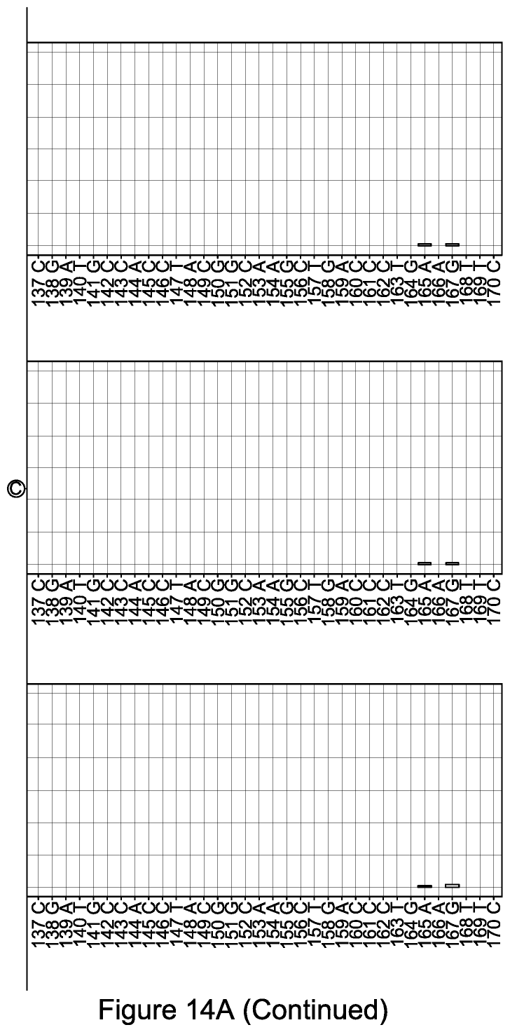

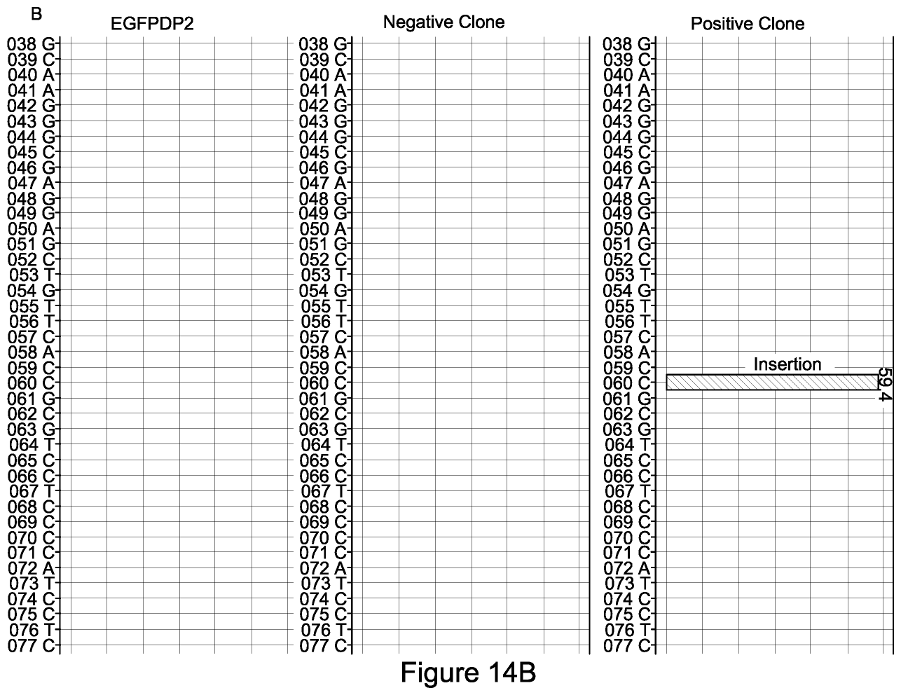

FIG. 14 shows confirmation of DLR-based gene conversion of nucleotide insertion and Indels analysis at a target region of this EGFPDP2 locus. Panel A shows overall views of insertion and deletion analysis of untargeted EGFPDP2 cells, a negative clone and a positive clone. Bar graphs show plots of frequencies of insertions and deletions at every nucleotide position of this 171 bp PCR amplification region for a single representative sample of each indicated situation. Results demonstrated that approximately 59.4% reads from this positive clone had an insertion at position “060C”, which corresponds to a position in which a nucleotide G was deleted at this locus. Remarkably no additional unwanted insertions or deletions were detected compared to background levels, compared to untargeted EGFPDP2 or a negative clone. Panel B shows magnification portions from indicated areas, clearly demonstrating a desired insertion at this desired site with a frequency of 59.4%. This result was surprising and important, as it provides a major advantage over current methods that often generated higher levels of insertions and deletions. Also important is that it also indicates that this DLR molecule triggered repair pathways that did not cause chromosome rearrangements.

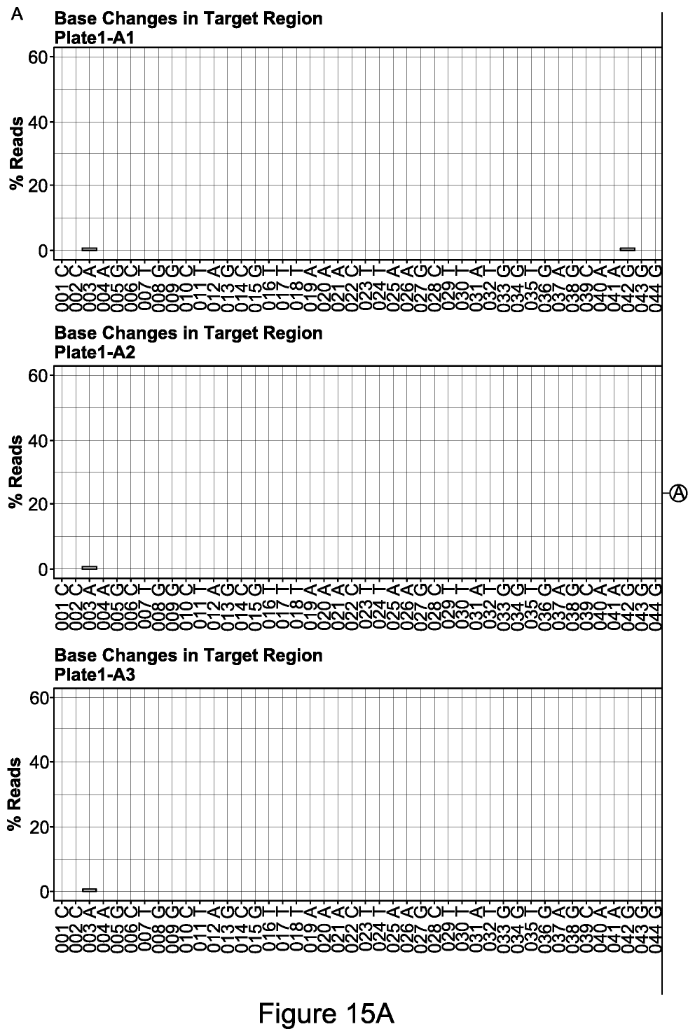

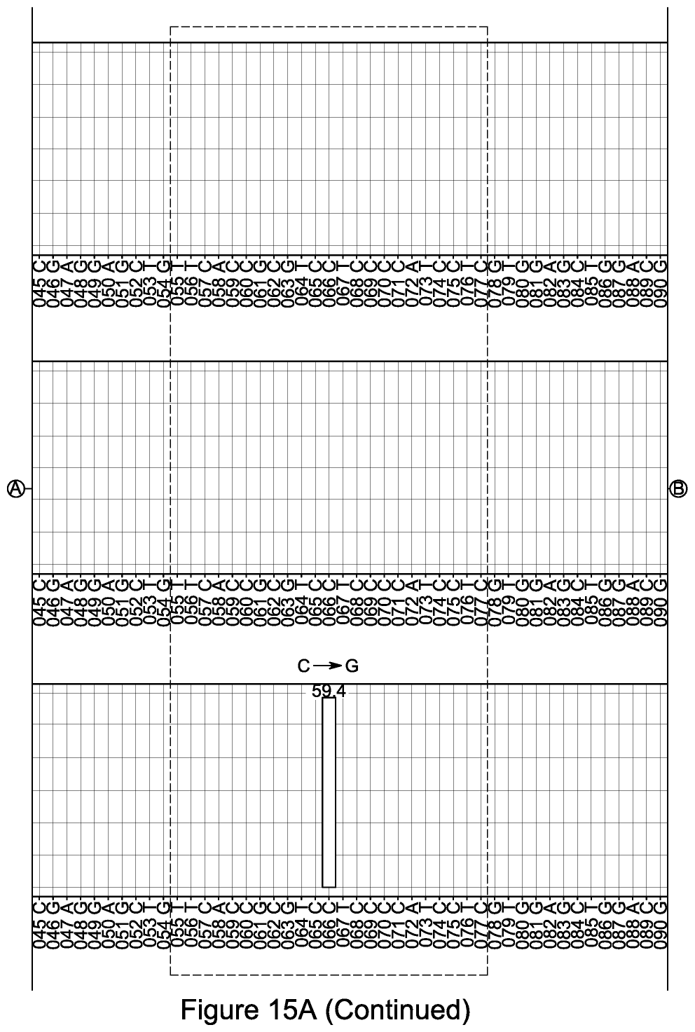

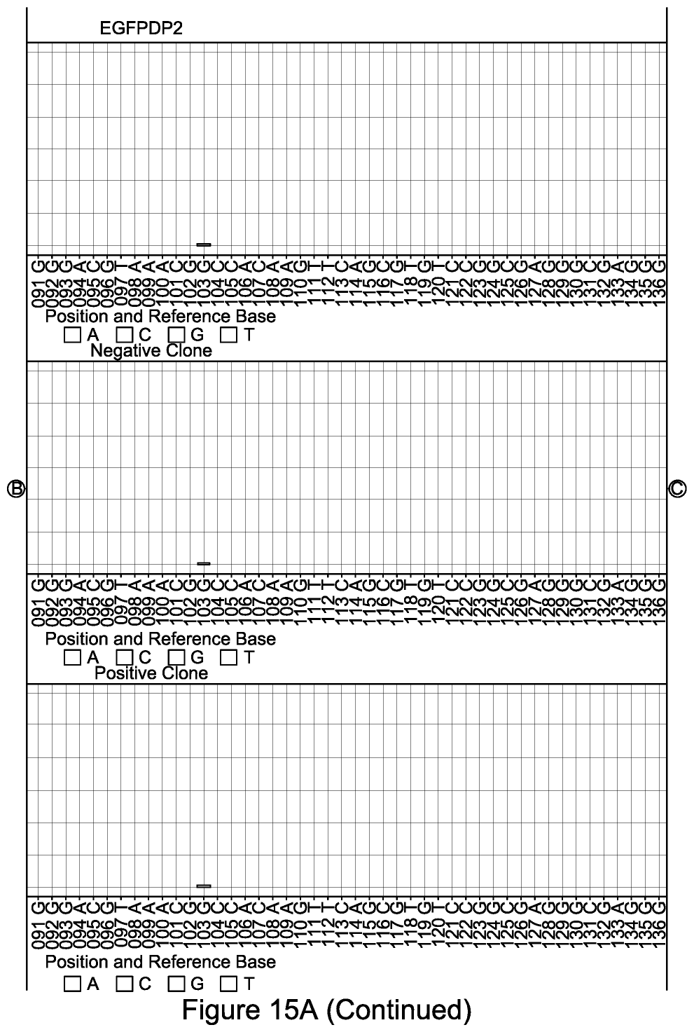

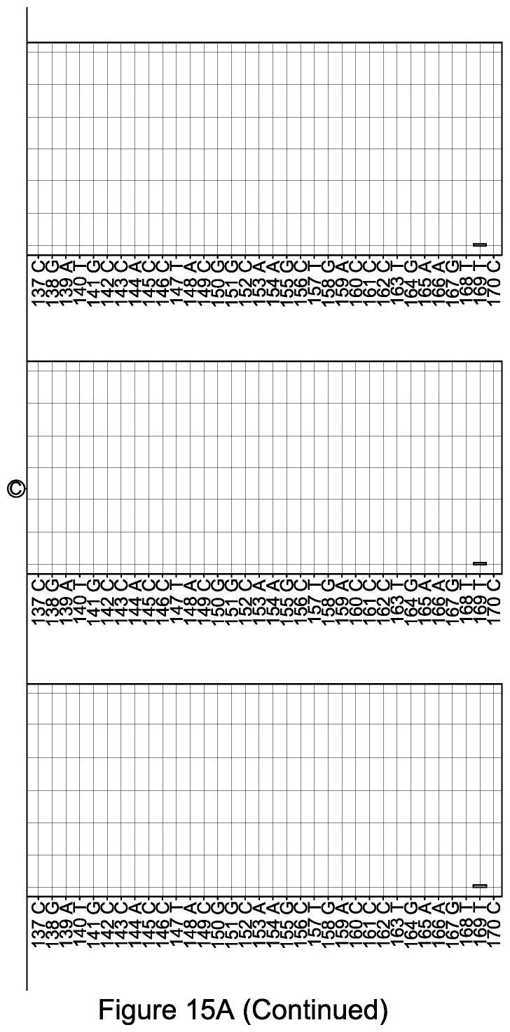

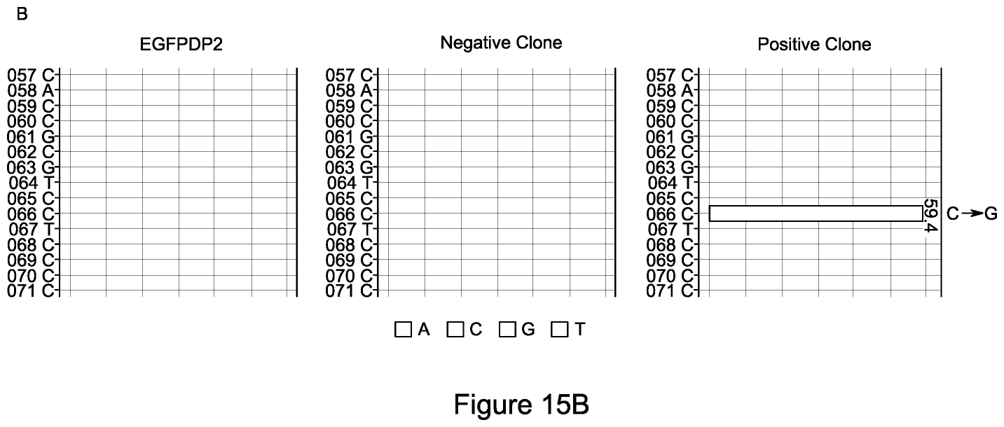

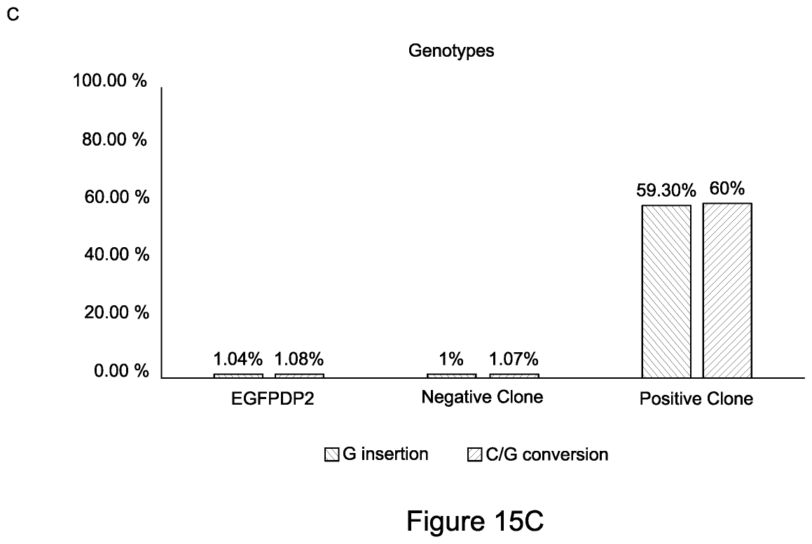

FIG. 15 shows confirmation of detected single nucleotide conversions at this target site as well as single nucleotide polymorphisms (SNPs) analysis within a target region of this EGFPDP2 locus. Panel A shows an overall views of SNPs analysis at these target sites of EGFPDP2 untargeted cells, a negative clone and a positive clone. Bar graphs plot frequencies of SNPs at every nucleotide position of this 171 bp PCR amplification region for a single representative sample of each indicated situation. This positive clone had a 59.4% C-to-G conversion at this designated C→G point mutation site. No additional point mutations or SNPs were introduced in this targeted region in this example of DLR-based targeted gene conversion. Compared to background levels as seen in two controls, no single nucleotide polymorphisms were apparently generated. Genotyping of C and G showed roughly equal percentages of C and G at this target site, suggesting that one chromosome of EGFPDP2 was repaired, which was consistent with Sanger sequencing results as shown in FIG. 13. Taken together, as illustrated in Panel C, DLR-based gene editing not only targets and repairs two mutations in EGFPDP2 in cells, but also resulted in an extremely low level of undesired genetic damages, including insertions, deletions, as well as point mutations.

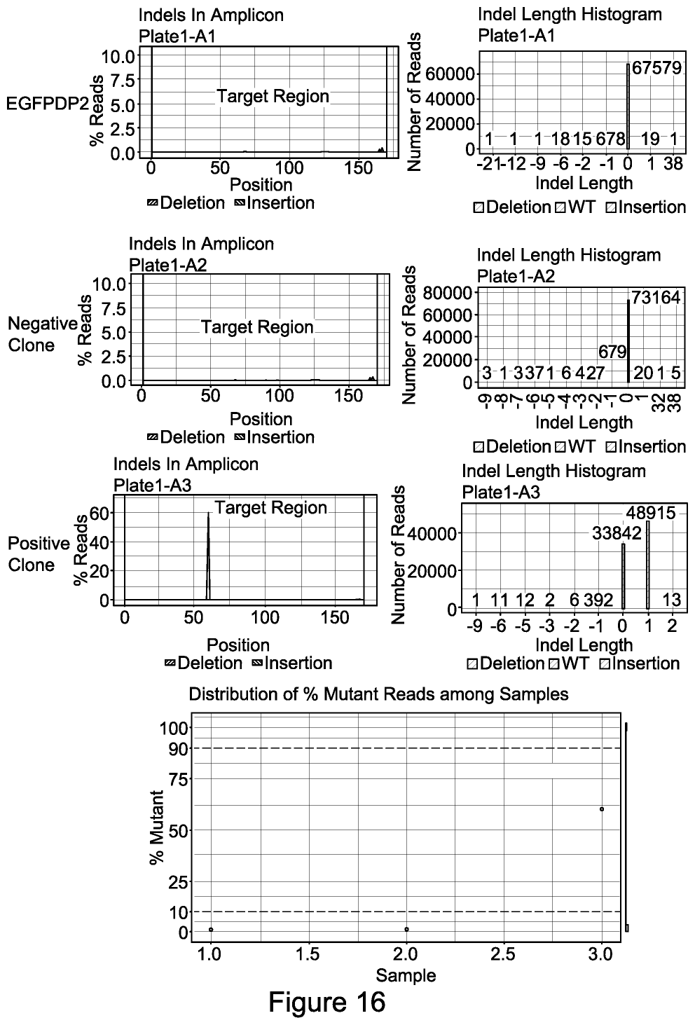

Lastly, FIG. 16 shows total reads numbers as well as reads lengths within this target region from each sample. Each sample yielded more than 50,000 sequencing reads, enabling a reliable next generation bioinformatic analysis. Both negative and positive clones had no large insertions or deletions after DLR-based gene targeting and repairing, demonstrating extremely low incidences of chromosome rearrangement comparable to an untargeted sample. Approximately 60% of analyzed sequence reads for this positive clone corresponded to the EGFP sequence, indicating that a conversion of homogenous EGFPDP2 to a heterozygous EGFPDP2/EGFP genotype had occurred in this clone.

In summary, DLR-based gene editing effectively targeted and corrected genetic mutations in presence of a correction template. In contrast to currently available systems, this approach provides the surprising findings that corrections occurred with an extremely low frequency of accompanying genetic background damage. These findings provide many indications for potential to use this system and provide many advantages as this approach demonstrates reduced risks of creating unwanted genetic mutations and increased safety profiles, particularly as compared to other currently available technologies.

### Example 2: Modification of an Endogenous Genomic Target: Codon 112 of Human ApoE by DLR-Based Gene Editing

In this example, human ApoE at codon 112 was targeted and edited by a specifically designed DLR molecule and a single stranded oligonucleotide template (i.e., a sequence modification polynucleotide). The human ApoE genotype is related to a risk of predisposition for developing Alzheimer's disease. Particularly, codon 112 encodes a critical residue relevant to Alzheimer's risk (or protection). This example describes development of a DLR-based gene editing system designed to convert a “T” to “C” at codon 112 in ApoE. In addition to being of potential clinical relevance, this target also exemplified usage of a naturally occurring target within a mammalian genome.

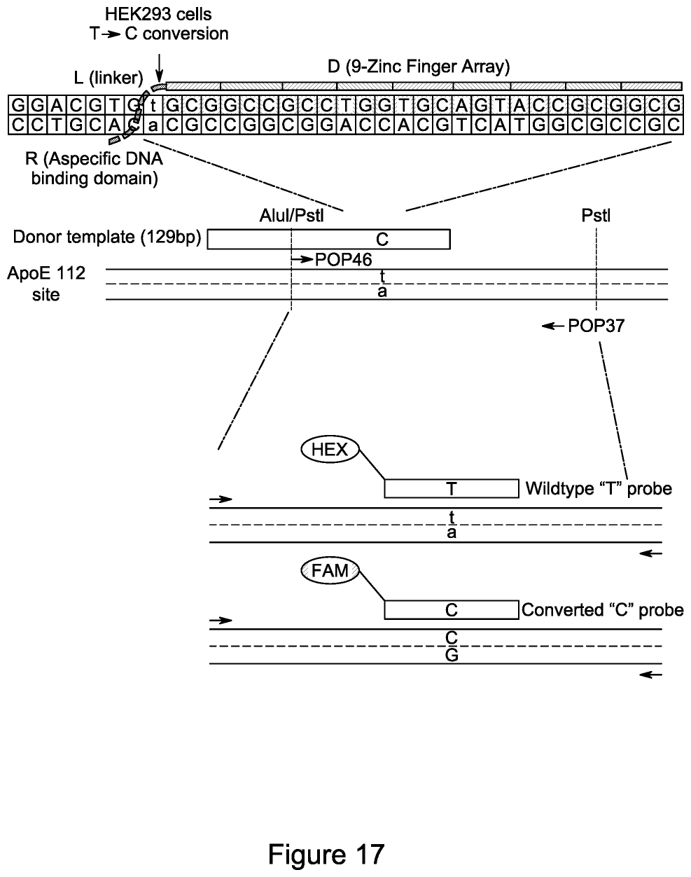

FIG. 17 illustrates an approach taken for this specific embodiment. This specific example aimed at gene editing of an endogenous genomic target around codon 112 of human ApoE in HEK293 cells. In this example, a DLR molecule, encoded on plasmid pb6 (full length DNA (SEQ ID NO. 21) cDNA (SEQ ID NO.: 87), DLR amino acid sequence (SEQ ID NO.: 88)), has a DNA recognition domain which was an array of 9 zinc-fingers, specifically designed to recognize 5′-GCGGCCGCCTGGTGCAGTACCGCGGCG-3′ (SEQ ID NO.: 8), a 27-nucleotide sequence on the leading strand of human ApoE. A targeted nucleotide “T” was displayed as a lowercase letter “t”, 5′ upstream of this binding site. An R element was designed to bind to an opposite strand, in this case the lagging strand, in a non-sequence-specific manner. In this embodiment, a donor template was used: a 129-nucleotide single stranded DNA oligonucleotide with a desired T→C substitution roughly located in the middle of this oligonucleotide. This single stranded donor template used herein is provided below as a sequence with an underlined and bold “C” to for T→C conversion.

(SEQ ID NO.: 22)
5′-CCCCGGTGGCGGAGGAGACGCGGGCACGGCTGTCCAAGGAGC
TGCAGGCGGCGCAGGCCCGGCTGGGCGCGGACATGGAGGACGTGC
GCGGCCGCCTGGTGCAGTACCGCGGCGAGGTGCAGGCCATGC-3′

Detections of genetic T→C conversion after DLR-based gene edition were performed by droplet digital PCR (ddPCR). Relative positions of a correction ssODN (i.e., sequence modification polynucleotide) and position of a common primer pair (POP46, POP37, SEQ ID NOS.: 24 and 80) are also indicated in FIG. 17. One common primer, POP46 was located inside this ssODN template (i.e., sequence modification polynucleotide) sequence, while POP37, located outside. Allele-specific probes conjugated with fluorophores FAM and HEX were designed to distinguish between “C” and “T” respectively. PstI restriction enzyme sites indicated were used in preparations for ddPCR reactions.

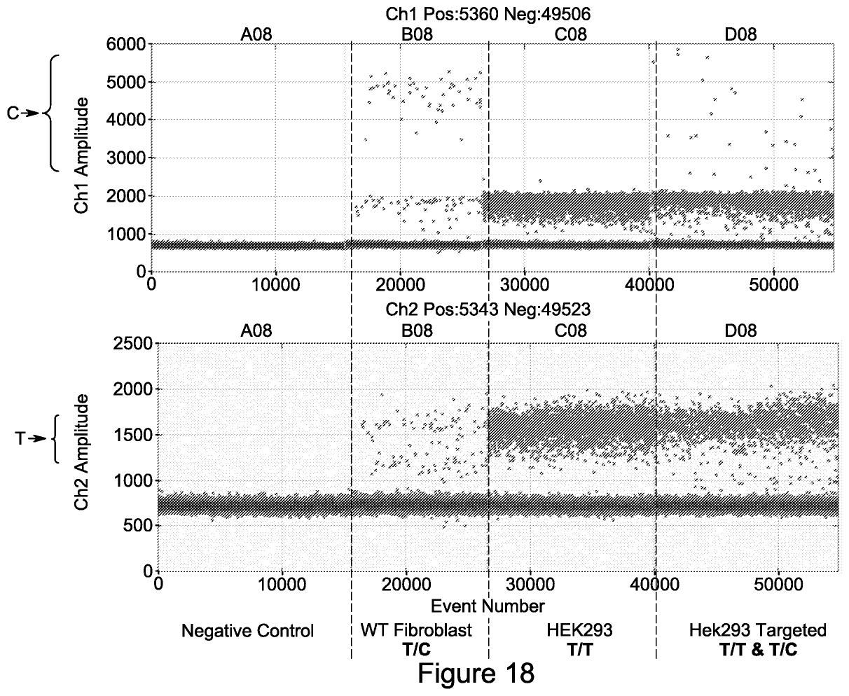

FIG. 18 demonstrates successful T→C genetic conversion at codon 112 of human ApoE as measured by ddPCR. In this example, after transfection of HEK293 cells with plasmid pb6 and this 129-nucleotide correction template, cells were allowed to recover and grow on complete culture medium, containing 15% FBS in DMEM, for seven days. After seven days genomic DNA was isolated and used in ddPCR analysis. Raw droplet data are shown as in FIG. 18 where these “C” droplets are displayed in the top panel; while “T” droplets were in the lower one. No DNA input was used as negative control, showing neither “C” nor “T” droplets. Wild type fibroblast was used as a positive control because of its heterozygous T/C genotype for codon 112 of human ApoE, showing both “C” and “T” droplets. The untargeted HEK293 only had “T” droplets, demonstrating homozygous T/T genotype. After HEK 293 transfected with pb6 and ssODN template (i.e., sequence modification polynucleotide), “C” droplets appeared after being targeted and edited by this DLR molecule in combination with a correcting template, demonstrating successful T→C genetic conversion at codon 112 of human ApoE.

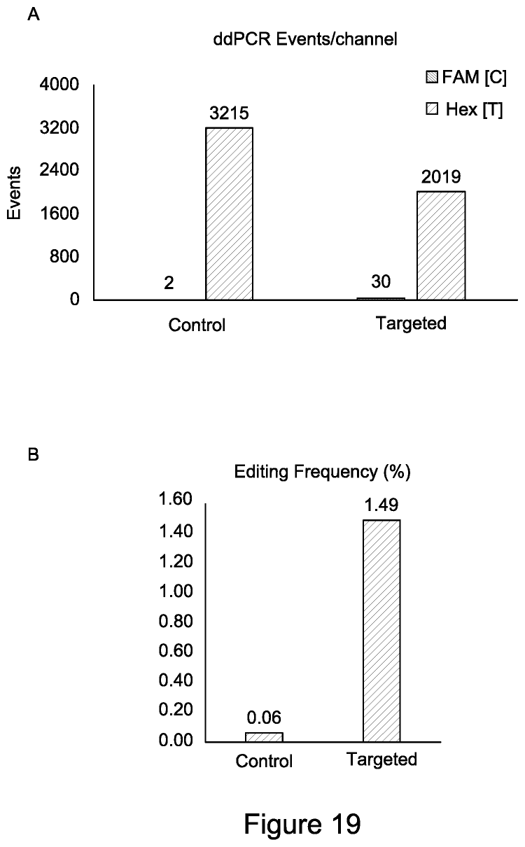

FIG. 19 shows T→C gene conversion frequencies as measured by ddPCR after DLR-based gene editing. Panel A shows absolute counts of individual droplet event per channel for untargeted (control) and targeted cellular pools. Panel B shows editing frequencies corresponding to cellular T to C conversion percentages, defined as the percentage of C droplet events divided by the sum of C and T droplet events. Here, this DLR-based gene editing achieved a 1.49% genetic conversion frequency compared to a background level of 0.06% of T-to-C conversion. Here, the background level is due to the method of detection employed. The frequency of conversion (1.49%) is significantly different from “background” conversions (0.06%).

In the present Example, next generation sequencing was performed to determine, in more detail, gene conversion frequencies and patterns and also potential generation of insertions, deletions, and unintended single nucleotide polymorphisms after DLR-based gene editing. In order to do so, next generation sequencing of targeted HEK293 pooled cells (and untransfected HEK293 as control) was performed. Genomic DNA was isolated and used as a template on which a 175-bp PCR amplicon surrounding ApoE codon 112 was generated by using a primer set of POP46 and POP37. Amplified PCR products from targeted HEK293 cells and control HEK293 cells were analyzed for indels and SNPs on an Illumina next generation sequencing platform (GENEWIZ, South Plainfield, NJ).

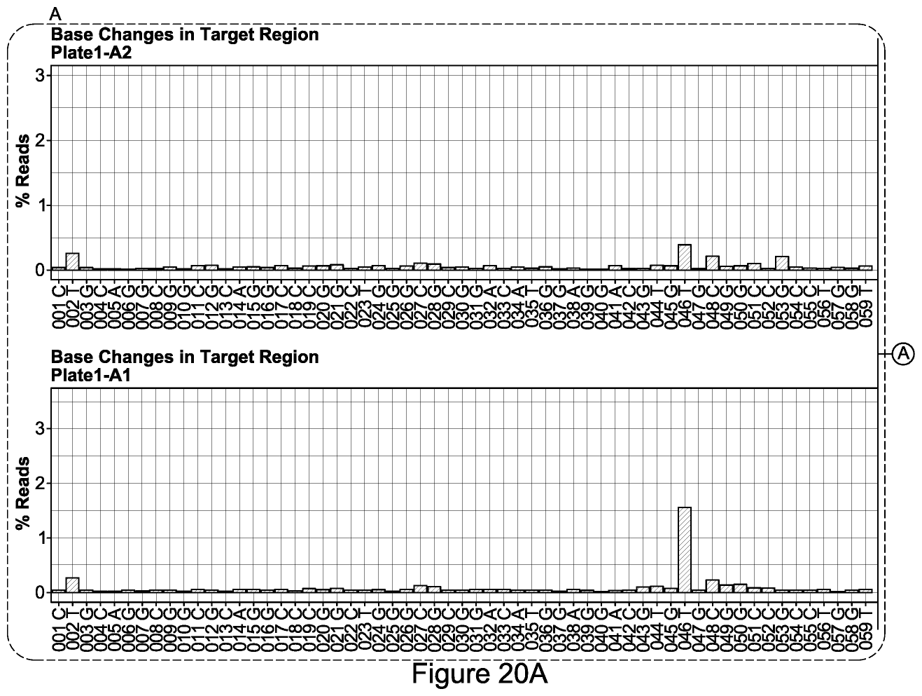

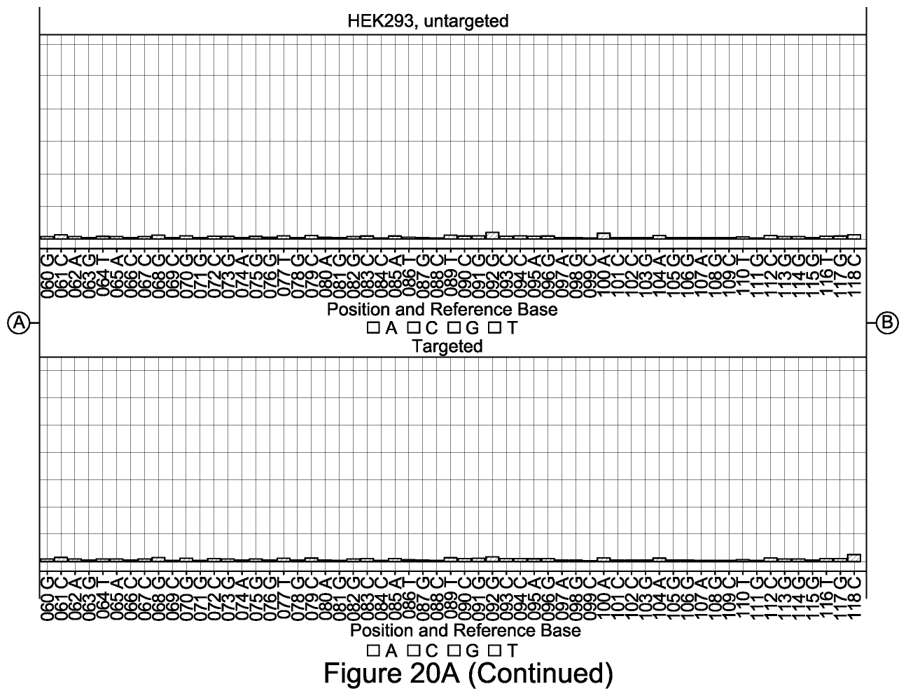

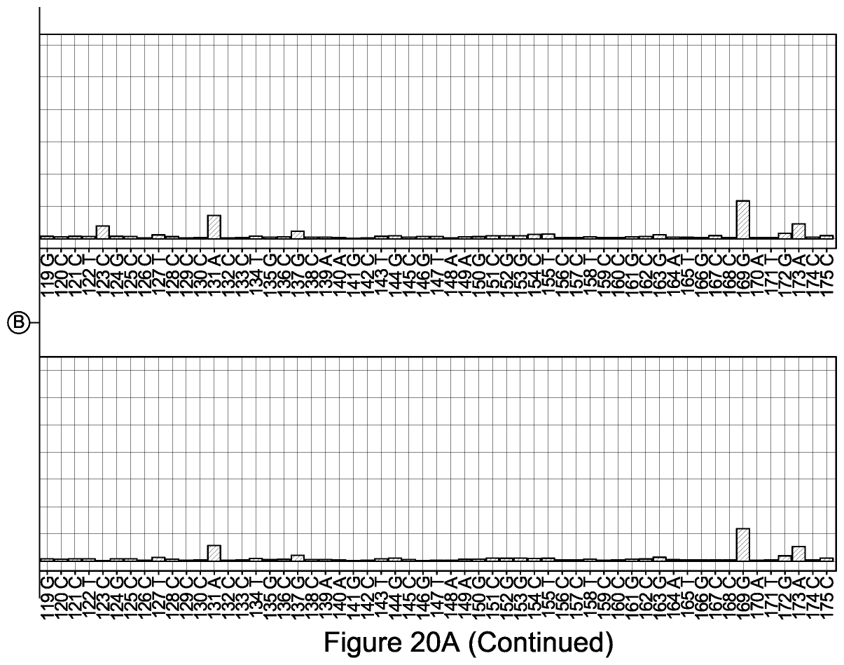

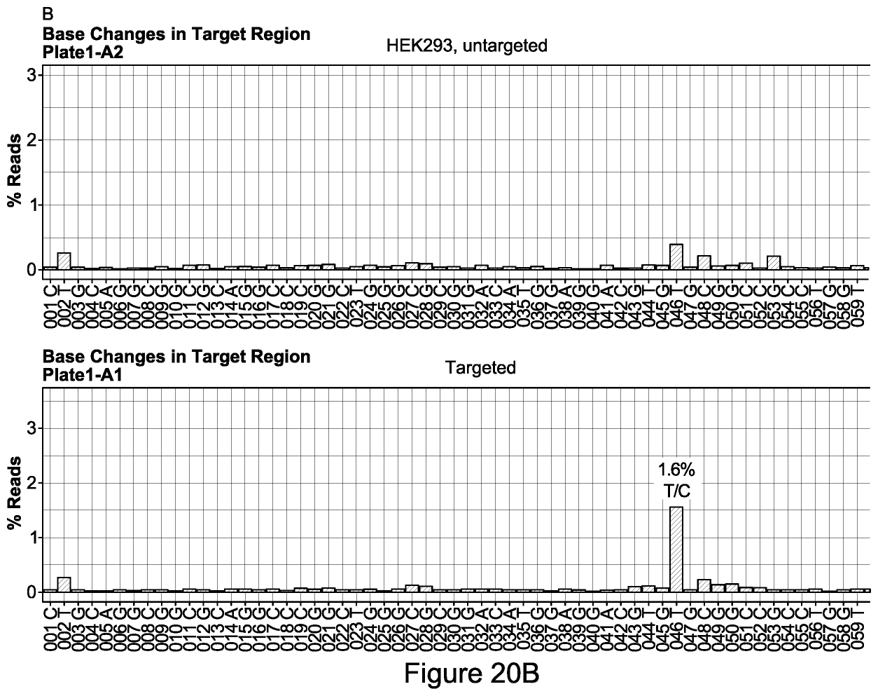

FIG. 20 shows confirmation of detection of single nucleotide T→C conversion at this target site as well as single nucleotide polymorphisms (SNPs) analysis within a target region of surrounding codon 112 of this ApoE locus. Panel A shows overall views of SNPs analysis at these target sites obtained with HEK293 untargeted cells, and targeted HEK293 pooled cells. Bar graphs plot frequencies of SNPs at each nucleotide position in this 175 bp PCR amplification region. Panel B is a magnified view of the portion close to this gene repair site. In this example cells transfected with pb6 and a correction template showed a T-to-C conversion at this expected nucleotide position with a frequency of 1.6%. Compared to non-transfected HEK293 cells, no other nucleotide conversions had occurred at a level significantly above background. A measured frequency of T-to-C conversion of 1.6% was consistent with a rate of 1.49% as determined by ddPCR. Comparing to untransfected cells, no obvious unwanted SNPs were detected.

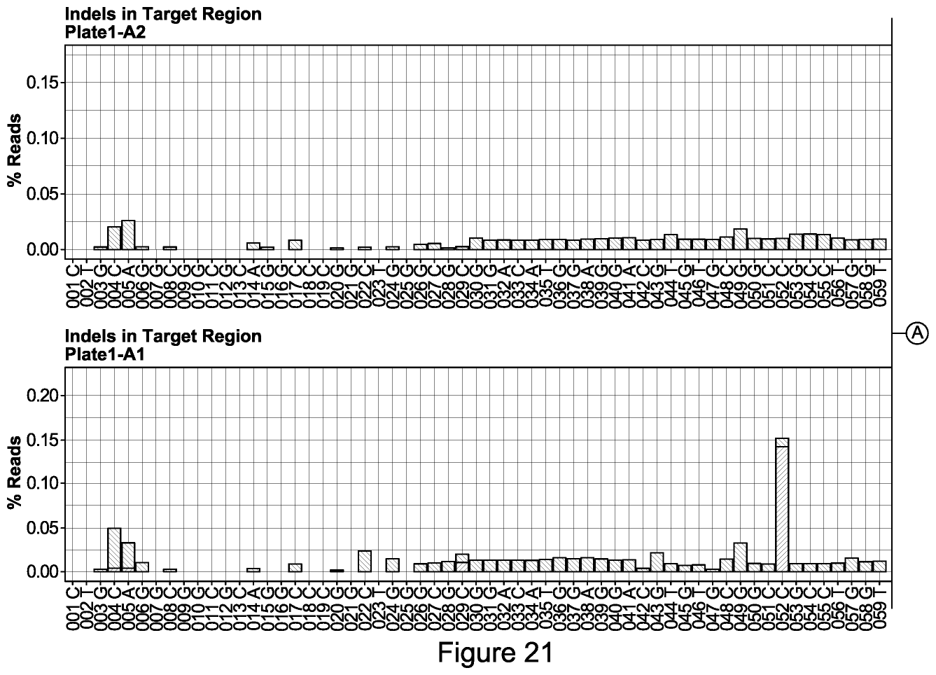

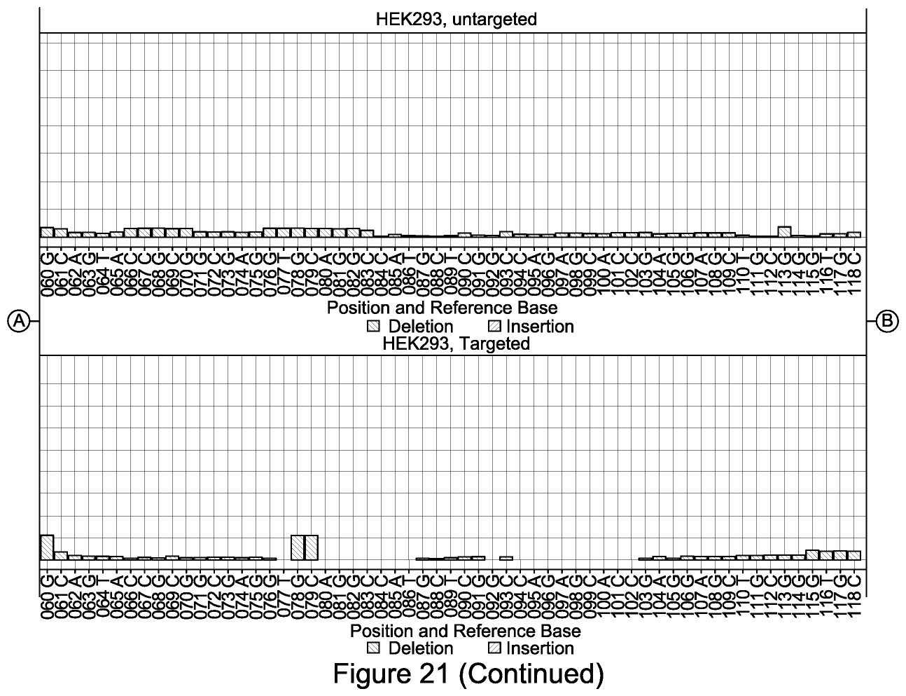

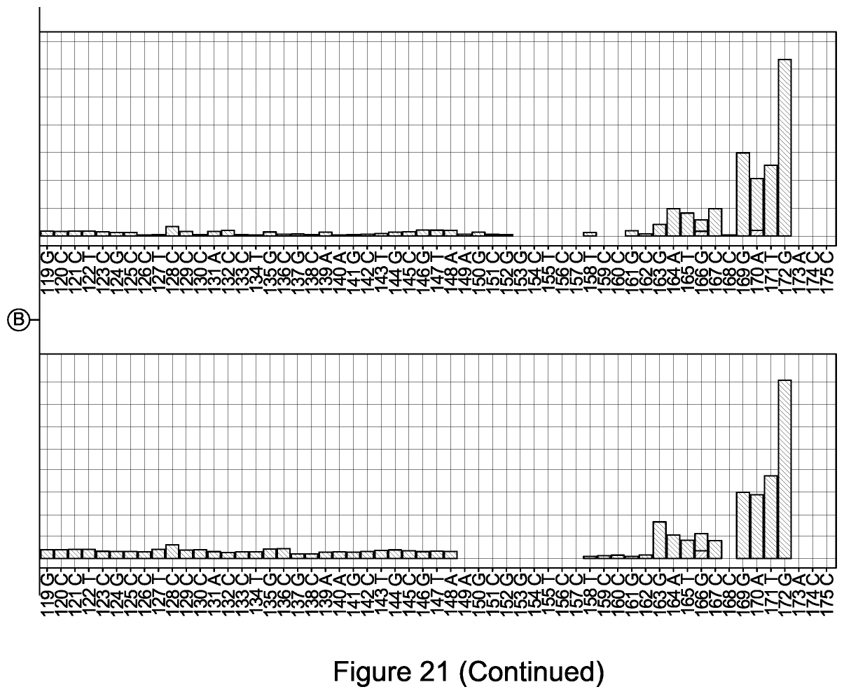

FIG. 21 shows insertion and deletion analysis around codon 112 of ApoE in this example, displayed a frequency plot of insertions and deletions analysis for untargeted HEK293 cells and targeted pooled HEK293 cells. Bar graphs plot frequencies of insertions and deletions at each nucleotide position of this 175 bp PCR amplification region. This indels analysis showed, in general, a very low frequency (<0.05%) of insertions and/or deletions. The highest level of change at any position was a nucleotide insertion of 0.15% at position 52 of this amplicon, which could also be observed with HEK293 controls and most likely reflected a technical artifact. In addition, patterns and frequencies of indels at each position from both targeted and untransfected HEK293 cells were no statistically significantly different and were considered to be within the error range and the detection limitations typical for the PCR and next generation sequencing method used.

Observations in this example were of paramount importance. A very low level of insertions and deletions as detected indicated that this present disclosure enables targeted gene conversion without potentially detrimental generation of insertions, deletions and/or undesired single nucleotide polymorphisms at significant levels. It also indicated that these DLR molecules triggered repair pathways that did not cause chromosome rearrangements.

While preceding disclosures indicated a very good safety profile, further results are being disclosed that illustrate that in clones derived from single transfected cells, a very high safety profile could also be observed. From a pool of transfected HEK293 cells, individual clones were grown and analyzed.

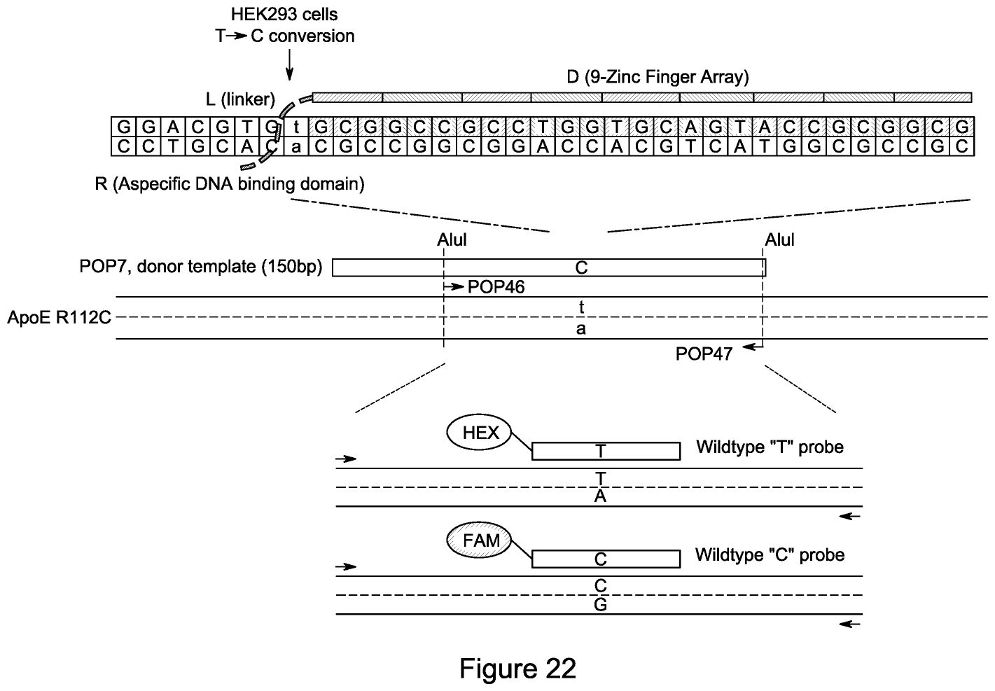

FIG. 22 illustrates key aspects for generation and analysis of ApoE codon 112 gene-converted HEK293 single cell clones. In this example, a DLR molecule encoded on plasmid pb6 (SEQ ID NO.: 21) was designed to target a 27-nucleotide site close to codon 112 of human ApoE. In addition, for this example, POP7, a 150-nucleotide-long donor single strand DNA oligonucleotide bearing a “C” substitution (to replace “T”) placed roughly in the middle of this template was designed as 5′-CCCCGGTGGCGGAGGAGACGCGGGCACGGCTGTCCAAGGAGCTGCAGGCGGCGCA GGCCCGGCTGGGCGCGGACATGGAGGACGTGCGCGGCCGCCTGGTGCAGTACCGCG GCGAGGTGCAGGCCATGCTCGGCCAGAGCACCGAGGAGC-3′ (SEQ ID NO.: 23). A C substitution is displayed both in bold and underlined. A common primer pair, POP46, 5′-CTGCAGGCGGCGCAGGC-3′ (SEQ ID NO.: 24), and POP47, 5′-CTCCTCGGTGCTCTGGCCGA-3′ (SEQ ID NO.: 25), was used for amplification for ddPCR-based detection, Sanger sequencing, and next generation sequencing, which are indicated. AluI restriction sites are indicated and AluI was included in sample preparation before ddPCR detection. Allele-specific probes conjugated with different fluorophores (FAM and HEX) are indicated for detection of “C” and “T”, respectively.

After transfection with pb6 and a correction oligonucleotide, cells were grown for 5 days in a complete growth DMEM medium containing 15% FBS. Thereafter, cells were dissociated with 0.25% trypsin/EDTA solution and plated in 96-well-plates at a density of 0.5-1.0 cells per well. Cells were allowed to grow into clones for about 3-4 weeks, and were then harvested.

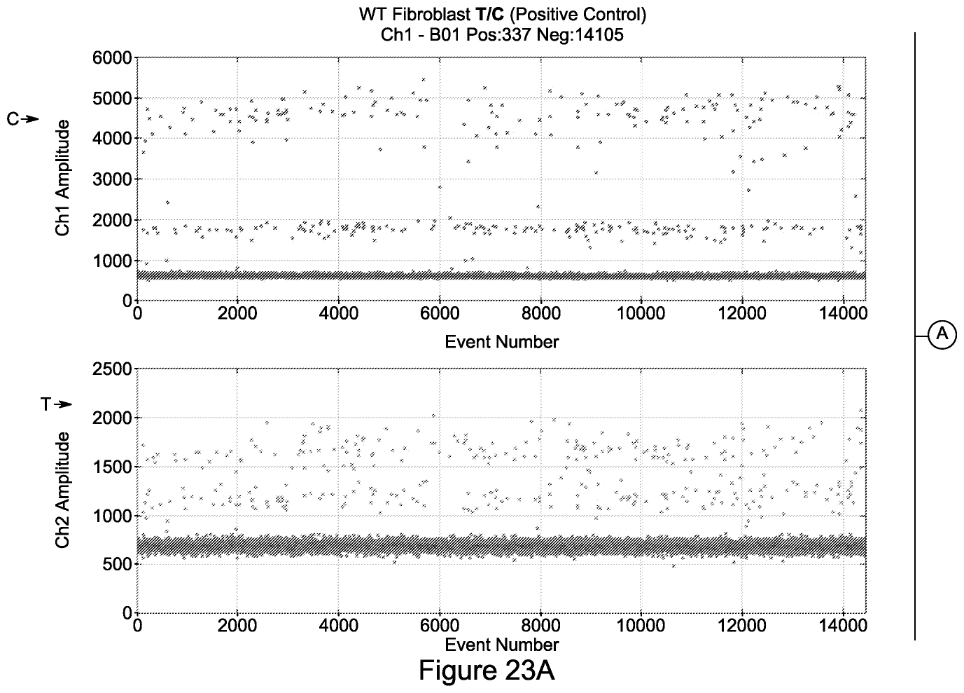

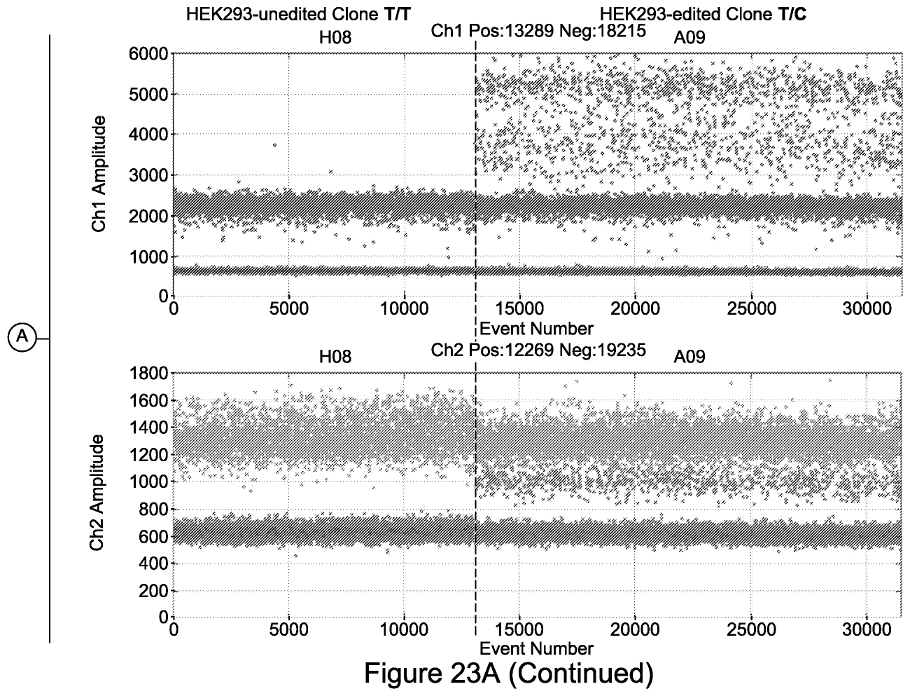

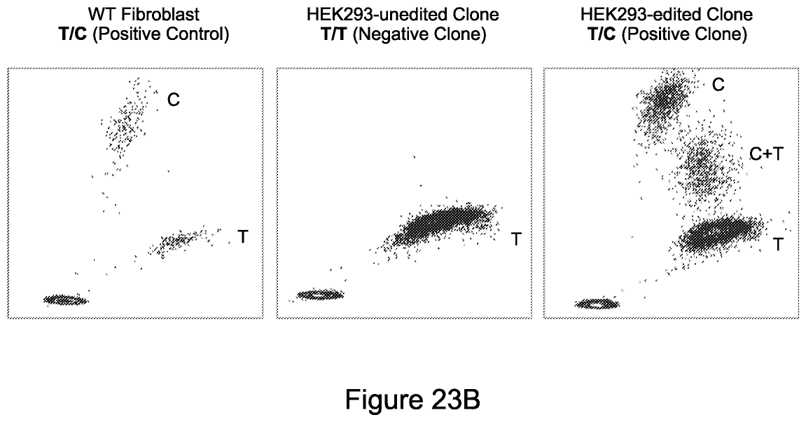

Chromosomal DNA was subsequently isolated using a solution-based DNA extraction method (Promega, Madison, WI). From three independent experiments, a total of 77 clones were analyzed by digital droplet PCR. Of these 77 clones, 8 were identified as having undergone a desired C-to-T conversion. FIG. 23, panel A shows representative ddPCR results of a converted clone together with controls. Human fibroblasts were used as a positive control, using their heterozygous T/C genotype, showing both “C” and “T” droplets. A negative clone used had no “C” droplets, while a positive clone post editing showed significant amounts of “C” droplets. Panel B shows the 2D plots representation of appearance of a “C” droplet population and a “C+T” population, in which both T and C alleles were detected simultaneously in these droplets.

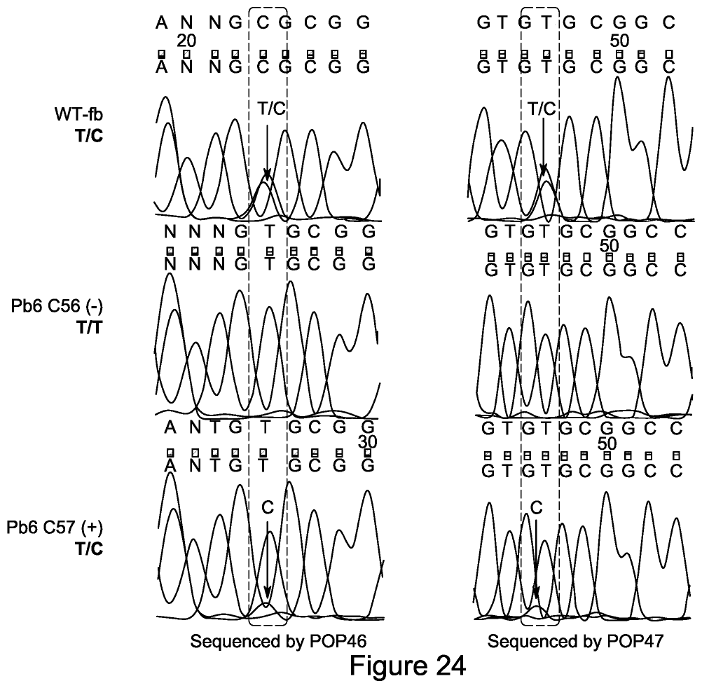

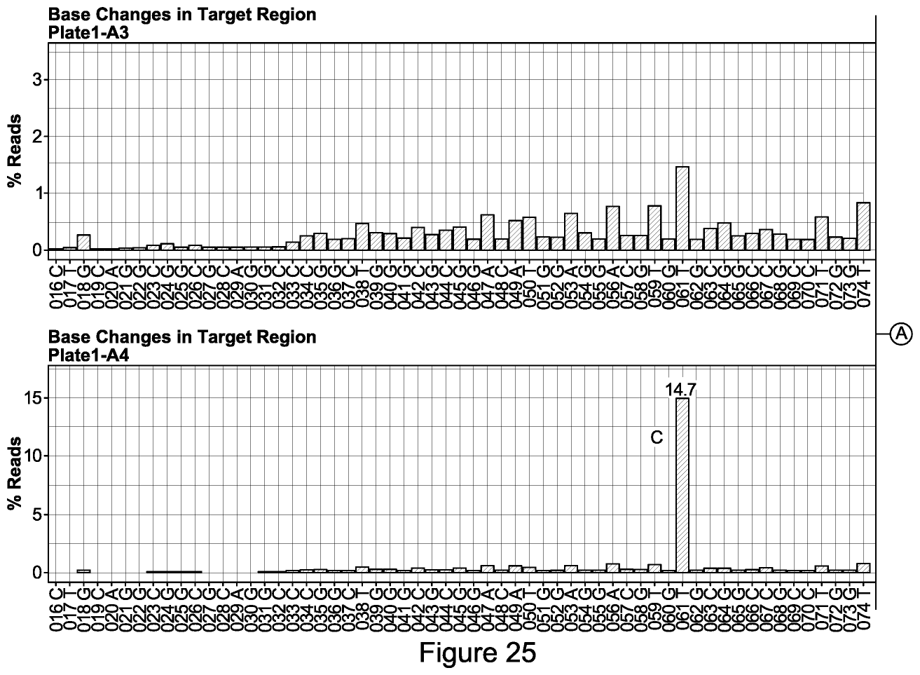

FIG. 24 illustrates Sanger sequencing results obtained with a representative gene converted clone. Using heterozygous fibroblasts as positive control, also a negative clone (C56) and a positive clone (C57) were sequenced using forward POP46 (SEQ ID NO.: 24) and reverse POP47 (SEQ ID NO.: 25) primers, respectively. A T→C conversion site was marked on the same position of all chromatograms. Heterozygous fibroblast showed both T and C spikes, demonstrating a heterozygous T/C genotype. Negative clone C56 only had one spike of T, demonstrating homozygous T/T genotype. Positive clone C57 showed a signal corresponding to a desired T-to-C conversion. In this example its signal did not have a 1-to-1 ratio as was observed with wild-type fibroblasts. One reason for this lower signal could be that HEK293 is known not to be diploid, but has an aberrant number of chromosomes. The actual number of copies of chromosome 19 (which harbors the ApoE gene) in this specific cell line may be higher than 2 and subsequently, conversion of a single copy of this gene could have resulted in a lower conversion ratio. These results demonstrated that a DLR molecule in combination with a suitable correction template could be used for targeted endogenous gene conversion in mammalian cells.

To further analyze effects of gene conversion in this clone, next generation sequencing was performed to determine, at which frequency(ies), insertions, deletions, and undesired single nucleotide polymorphisms occurred. Genomic DNA derived from individual ApoE codon 112 converted clones was used. In this example, a 108 base-pair PCR amplicon surrounding ApoE codon 112 was generated and analyzed using an “Amplicon-EZ” procedure on an Illumina 2×250 base-pair platform (GENEWIZ, South Plainfield, NJ). Genomic DNA from an unconverted HEK293 negative clone was also isolated and used as a control.

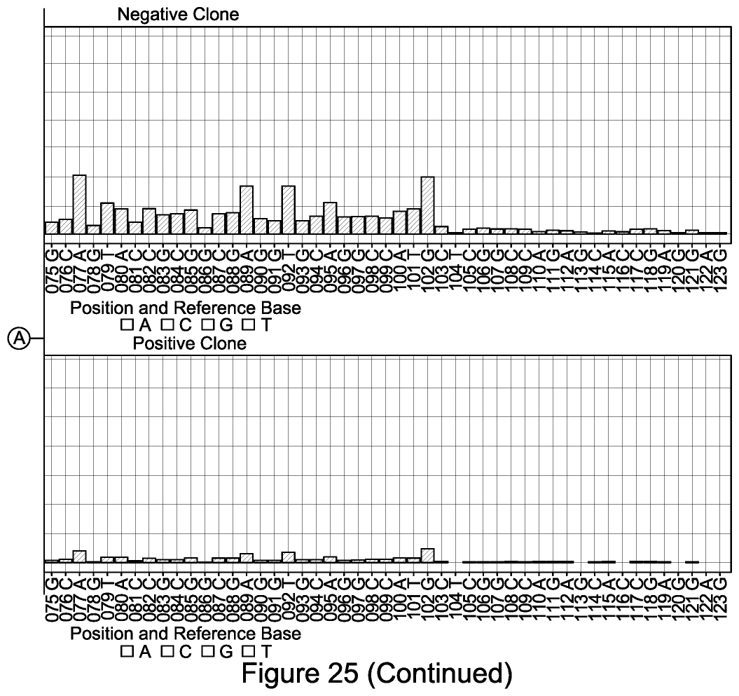

FIG. 25 shows a Single Nucleotide Polymorphisms (SNPs) Analysis result as obtained with an ApoE T→C positive clone versus an unconverted negative clone (i.e., a clone that was treated under the same conditions as a positive clone, but has an unconverted genotype). Approximately 14.7% of reads corresponded to a desired T-to-C conversion (lower panel). Without being bound by any particular theory, it is possible that a reason that the conversion ratio is not closer to a 50% ratio is because HEK293 cells have more than two copies of chromosome 19. The upper panel shows background signals for a parental, unconverted HEK293 clone. No additional unwanted single nucleotide polymorphisms were detected compared to background levels (compared with HEK293).

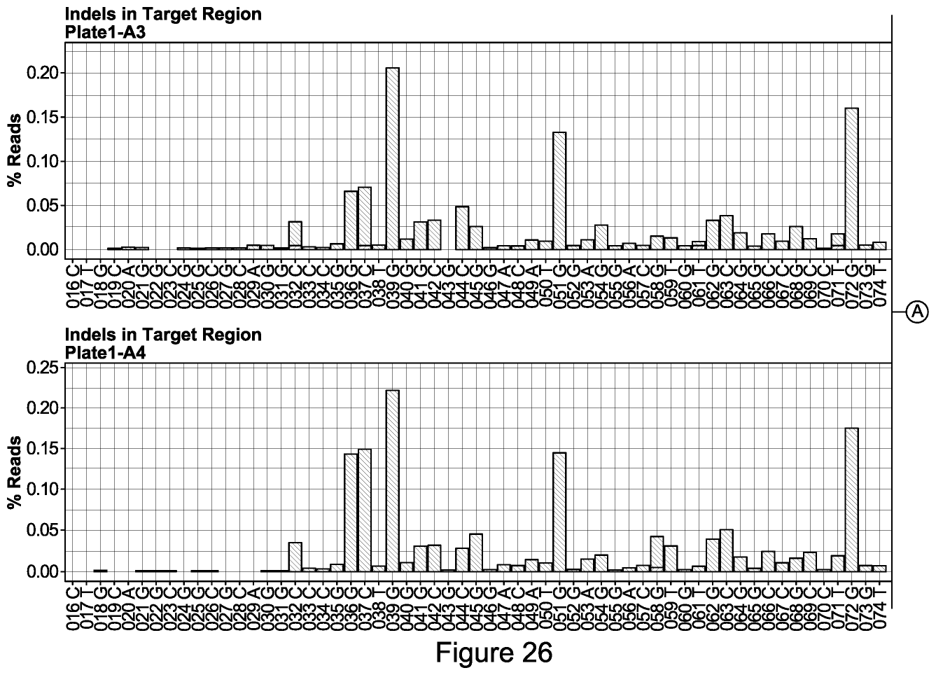

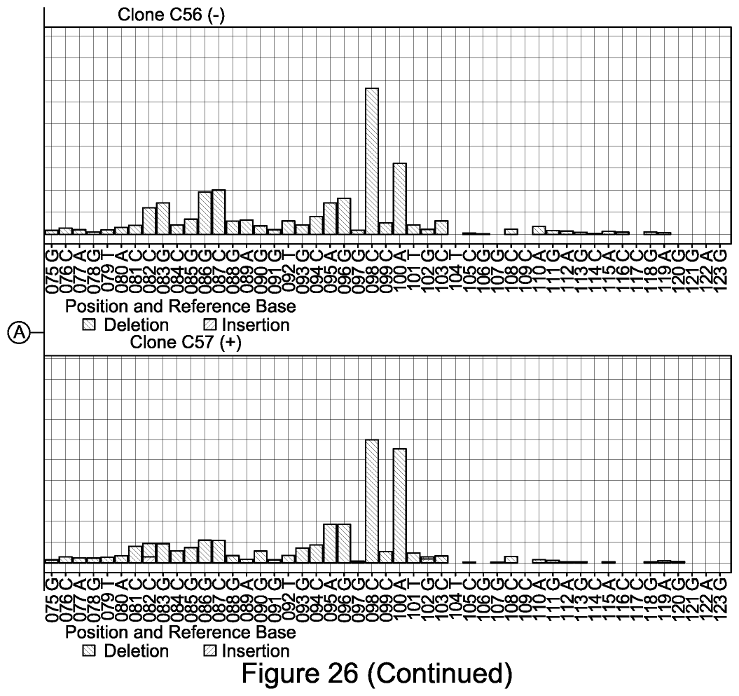

FIG. 26 illustrates an insertion and deletion (Indels) analysis, comparing a T→C converted clone to a unconverted negative HEK293 clone. Strikingly no insertions were observed and deletions remained at frequencies lower than 0.2% with no significant difference between these converted and unconverted cells. This result was important, as it pointed at a major advantage over current methods that often generate higher levels of insertions and deletions. It also indicated that these DLR molecules triggered repair pathways that did not cause chromosome rearrangements.

### Example 3: On-Target and Off-Target Analysis by Genome-Wide Unbiased Circular Sequencing

An aim of gene editing can be to correct mutations in endogenous genes to cure or prevent human diseases. Therapeutic applications in humans depend on high levels specificity and excellent safety profiles. Therefore, demonstrating on-target specificity and identifying off-target effects in human and other eukaryotic cells is critically important. In this example we used a circular deep sequencing method to confirm on-target gene conversion at codon 112 of human ApoE while simultaneously analyzing potential off-target insertions of the correction template on a genome-wide scale.

There was a need to have an unbiased method that could analyze desired and undesired events at a target locus, as well as analyze potential off-target events in a genome. As shown in above examples, single nucleotide polymorphism, insertion and deletion analysis by next generation sequencing was already indicating that undesired and off-target effects were happening only at very low frequencies when using a DLR-based DNA editing system. In order to fulfill this need for additional analysis, a novel “Circular-Seq” method was developed and applied. Goals of this method were to address whether DLR-based gene editing created undesired mutations at a target locus (and a target site) and/or resulted in correction templates being integrated at off-target sites.

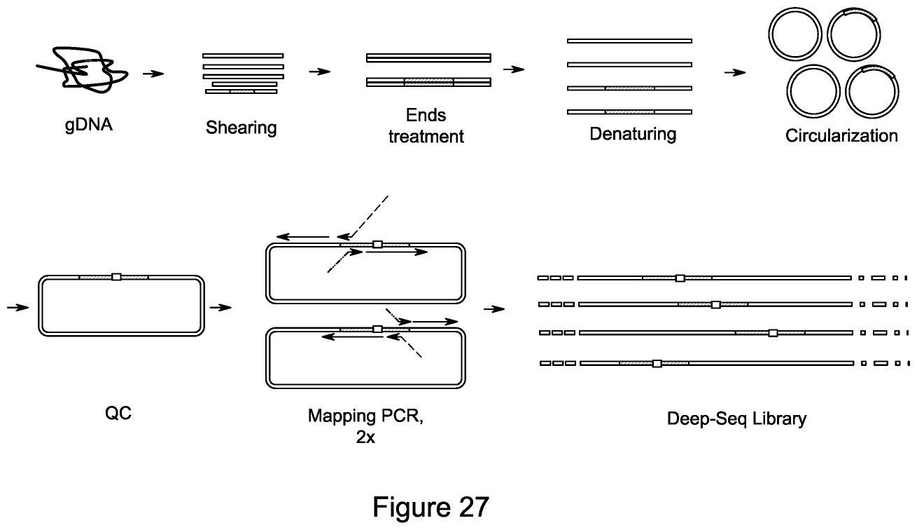

FIG. 27 shows an overview of this Circular-Seq method. Isolated genomic DNA from a gene-converted clone was extracted and randomly sheared to fragments of about 500 bp in length by sonication. This length was chosen so that donor template sequences or corrected sequences could reside within DNA fragments. Sheared DNA fragments were subsequently melted into single strands, followed by ligation done by using single strand DNA ligase to form single strand DNA circles. Un-circulated or double stranded DNA fragments were removed by using exonucleases. Circular single strand DNA (ssDNA) was then utilized as a PCR template. PCR primers were designed facing away from each other to amplify entire circularized ssDNA templates. Therefore, every amplicon comprises a sequence of this target region and joint flanking sequences outside this specific target site depending on its circular ssDNA template. For next generation sequencing on an Illumina platform, special tags were added to 5′ ends of each primer. Hi-fidelity PCR reactions were subsequently performed with Phusion DNA polymerase (New England Biolabs, Ipswich, MA) by making use of a set of tagged primers, POP58 5′-ACACTCTTTCCCTACACGACGCTCTTCCGATCTTCGGCCAGAGCACCGAGGAG-3′ (SEQ ID NO.26) and POP59 5′-GACTGGAGTTCAGACGTGTGCTCTTCCGATCTGCATGGCCTGCACCTCGC-3′ (SEQ ID NO.: 27). PCR products were then purified and DNA sequences were determined by next generation sequencing. Since each set of primers was back-to-back and facing away from each other, PCR products could continue through flanking sequences (at the end of donor or target sites) and only stop at their opposing primer-binding site.

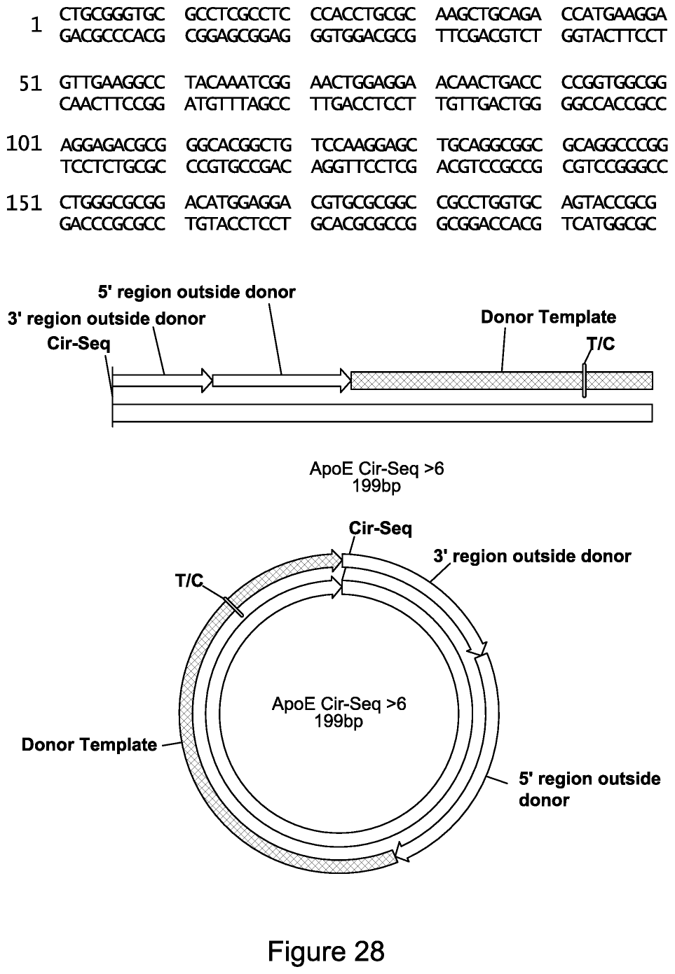

FIG. 28 illustrates an exemplary molecular structure and interpretation of one sequence read from circular sequencing to identify 5′-sequences and 3′-sequence relative to a donor template sequence that was integrated into a genome. In this circular display, as an example, when a random fragment was long enough and contained both a 5′ proceeding and a 3′ proceeding sequence, after circularization, this sequencing reaction could determine these sequences using outward directed primers. The middle panel is a linear representation and the upper panel shows an actual example sequence obtained through this analysis. Using bio-informatic tools, sequences containing a T→C conversion could be identified and further analyzed. Bio-informatics could also be used to identify any sequences that deviated from an expected ApoE sequence, which would have indicated potential off-target effects.

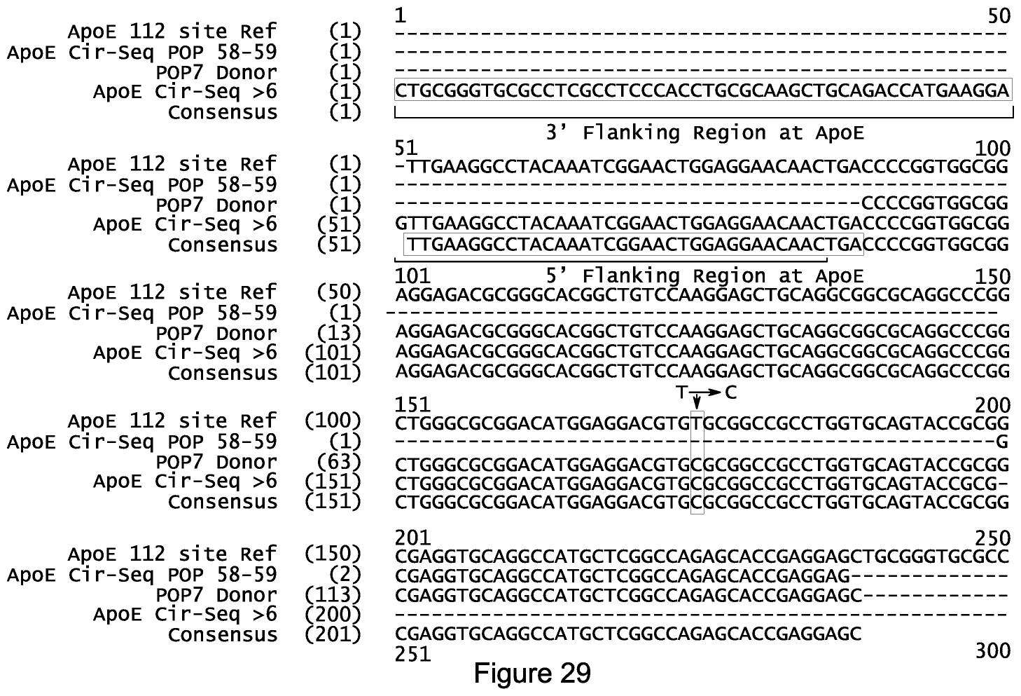

FIG. 29 illustrates a sequence alignment output from bio-informatics analysis of this example. Five sequences are shown: (1) ApoE sequence of HEK293; (2) back-to-back primers binding sequence; (3) donor template, (4) sequence of a representative circular deep sequencing read (ApoE Cir-Seq >6); (5) consensus sequence generated from circle sequencing reads. In this example, this ApoE Cir-Seq >6 sequence contained, from 5′ to 3′, a 3′ flanking region of this ApoE donor followed by 5′ flanking region of this ApoE donor, then a partial sequence exactly the same as this donor template with a desired T→C conversion (under the arrow). Only sequences that were found corresponded to ApoE sequences. No sequences were obtained that differed from ApoE sequences that would have been an indication of potentially off-target integration of correction templates.

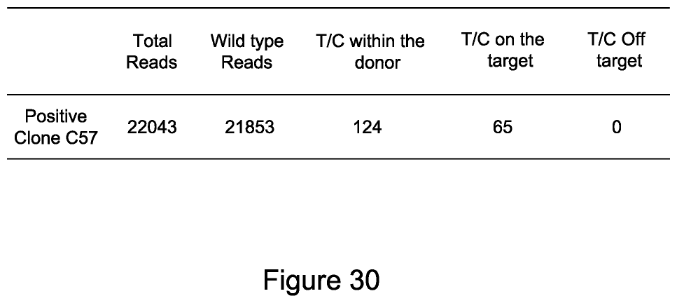

FIG. 30 shows a numerical analysis of sequence reads obtained by circular deep sequencing using chromosomal DNA derived from a positive clone. The total number of sequence reads was 22,043; of those reads, 124 contained a desired T→C conversion and all remaining 21,853 reads were wild type reads. No other sequences indicative of insertions, deletions, SNPs or other rearrangements were observed. Since HEK293 is known not to be diploid, but to have a higher number of chromosomes, this may have impacted this observed ratio. Key is that no other sequences besides wild type and a desired C-to-T conversion were observed. Out of 124 reads containing the C-to-T conversion, 65 were long enough to extend beyond the sequence of the oligonucleotide used. If integration of a correction template had occurred at a site other than an ApoE site, flanking DNA sequences would have been different from ApoE sequences. All sequences obtained from these 65 reads corresponded to expected ApoE sequences, indicating that no off-target integration had happened.

### Example 4: Modification of an Endogenous Genomic Target at Codon 158 of ApoE by a DLR-Based System

In this example, human ApoE at codon 158 was targeted by a specifically designed DLR molecule along with an ssODN correction template (i.e., sequence modification polynucleotide) to convert C to T. ApoE gene variant ApoE4 encodes two arginine (Arg) residues at amino acid positions 112 and 158 (Arg112/Arg158), and is the largest and most common genetic risk factor for late-onset Alzheimer's disease. Other ApoE variants with Cysteine (Cys) residues in positions 112 or 158, including ApoE2 (Cys112/Cys112) and ApoE3 (Cys112/Arg158), are presumed to decrease Alzheimer's disease risk than ApoE4. This example demonstrates use of a DLR-based genetic editing system to correct disease-relevant mutations in mammalian cells. In addition to being of potential clinical relevance, this target also provides an additional example of use of a naturally occurring endogenous target within a mammalian genome, combined with an engineered system provided by the present disclosure.

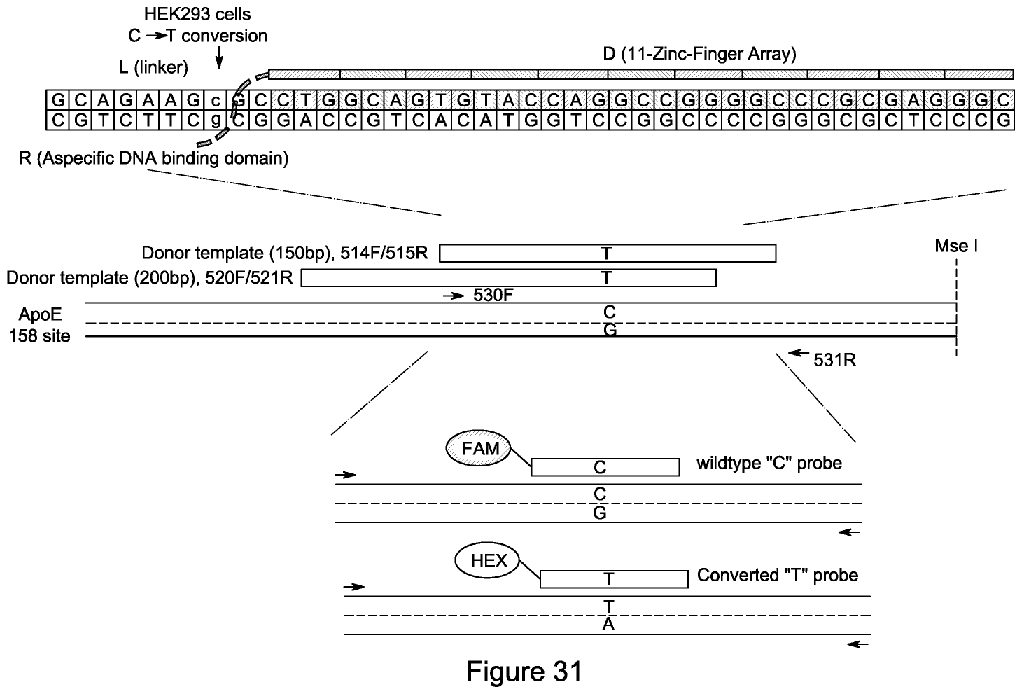

FIG. 31 illustrates an approach taken for this Example. This specific example aimed at gene editing of an endogenous genomic target around codon 158 of human ApoE in HEK293 cells. For this embodiment a DLR molecule was designed and encoded on plasmid pb41 (full length DNA (SEQ ID NO.28), cDNA (SEQ ID NO.: 89), and DLR amino acid sequence (SEQ ID NO.90)) that encompassed as DNA recognition domain an array of 11 zinc fingers, specifically designed to recognize a 33-nucleotide sequence, 5′-CTGGCAGTGTACCAGGCCGGGGCCCGCGAGGGC-3′ (SEQ ID NO.: 10) on the leading strand of the ApoE gene. A targeted nucleotide “C” was displayed as lowercase letter “c”, 5′ upstream of this binding site.

In this example an R element was designed to bind to the opposite strand, in this case the lagging strand, in a non-sequence-specific manner. In this embodiment donor templates were used that included a 150-nucleotide DNA oligonucleotide (514 Forward (SEQ ID NO.: 29); 515 Reverse (SEQ ID NO.: 30)) or a 200-nucleotide DNA oligonucleotide (520 Forward (SEQ ID NO.: 31); 521 Reverse (SEQ ID NO.: 32)) with a desired C→T substitution located within these oligonucleotides. Detections of genetic C→T conversion after DLR-based gene editing were applied by ddPCR. Relative positions of a correction ssODN (i.e., sequence modification polynucleotide) and positions of a common of primer pair (530F, 530R, SEQ ID No.82, and 83) are also indicated in FIG. 31. One common primer, 530F, located inside these ssODN templates (i.e., sequence modification polynucleotides), while the other, 531R, outside. Allele-specific probes conjugated with fluorophores FAM and HEX were designed to distinguish between of “C” and “T” respectively. An MseI restriction enzyme site is indicated that could be used in preparations for ddPCR reactions.

Four ssODN sequence modification polynucleotides for genetic C→T conversion of codon 158 of human ApoE appear from top to bottom below, respectively. Converting nucleotide “T,” on forward donor templates, or “A” on reverse templates respectively are marked in underlined bold letters.

Donor template, 514 Forward (SEQ ID NO.: 29), is displayed as follows:

GCGGGTGCGCCTCGCCTCCCACCTGCGCAAGCTGCGTAAGCGGCT
CCTCCGCGATGCCGATGACCTGCAGAAGTGCCTGGCAGTGTACCA
GGCCGGGGCCCGCGAGGGCGCCGAGCGCGGCCTCAGCGCCATCCG
CGAGCGCCTGGGGCC.

Donor template, 515 Reverse (SEQ ID NO.: 30), is displayed as follows:

GGCCCCAGGCGCTCGCGGATGGCGCTGAGGCCGCGCTCGGCGCCC
TCGCGGGCCCCGGCCTGGTACACTGCCAGGCACTTCTGCAGGTCA
TCGGCATCGCGGAGGAGCCGCTTACGCAGCTTGCGCAGGTGGGAG
GCGAGGCGCACCCGC.

Donor template, 520 Forward (SEQ ID NO.: 31), is displayed as follows:

CCGGCTGGGCGCGGACATGGAGGACGTGCGCGGCCGCCTGGTGCA
GTACCGCGGCGAGGTGCAGGCCATGCTCGGCCAGAGCACCGAGGA
GCTGCGGGTGCGCCTCGCCTCCCACCTGCGCAAGCTGCGTAAGCG
GCTCCTCCGCGATGCCGATGACCTGCAGAAGTGCCTGGCAGTGTA
CCAGGCCGGGGCCCGCGAGG.

Donor template, 521 Reverse (SEQ ID NO.: 32), is displayed as follows:

CCTCGCGGGCCCCGGCCTGGTACACTGCCAGGCACTTCTGCAGGT
CATCGGCATCGCGGAGGAGCCGCTTACGCAGCTTGCGCAGGTGGG
AGGCGAGGCGCACCCGCAGCTCCTCGGTGCTCTGGCCGAGCATGG
CCTGCACCTCGCCGCGGTACTGCACCAGGCGGCCGCGCACGTCCT
CCATGTCCGCGCCCAGCCGG.

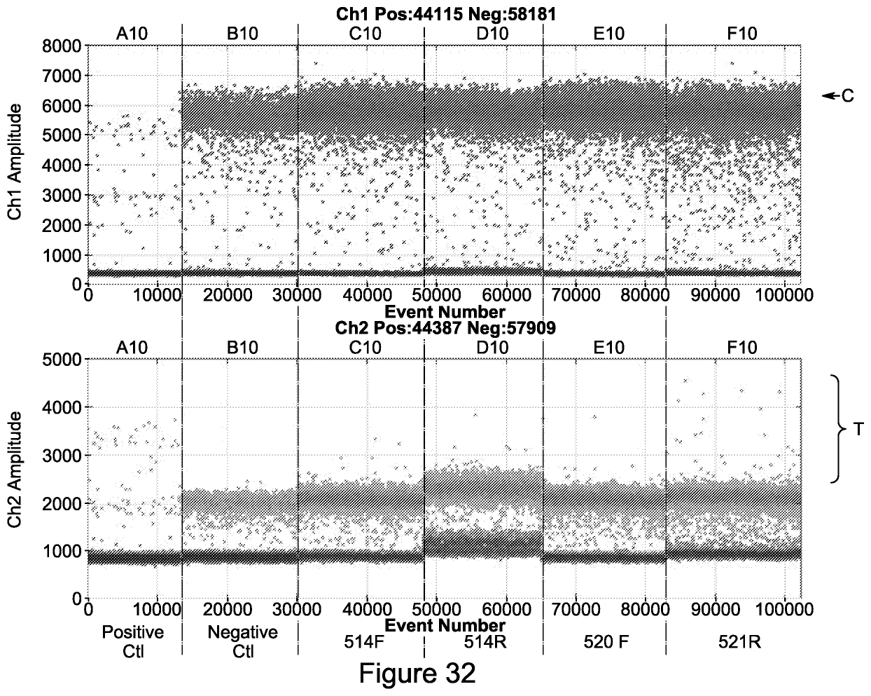

FIG. 32 demonstrates successful C→T genetic conversion at codon 158 of human ApoE as measured by ddPCR. In this example, after transfection of HEK293 cells with plasmid pb41 and one of four ssODN sequence modification polynucleotides, cells were allowed to recover and grown on complete DMEM growth medium containing 15% FBS for 7 days. After 7 days genomic DNA was isolated and used in digital droplet PCR analysis to determine “C” or “T” of ApoE codon 158. Raw droplet data are shown as in FIG. 32 where the “C” droplets were displayed in the top panel; while “T” droplets the lower one. Fibroblast cell line AG21158 was used as a positive control (heterozygous T/C genotype at codon 158 of human ApoE), showing both “C” and “T” droplets. The AG21158 fibroblast cell was obtained from Corriell Institute with ApoE genotype of E2/E3. HEK293 is used as a negative control that only has “T” droplets, corresponding to a homozygous C/C genotype. After HEK 293 was transfected with pb41 and four ssODN templates (i.e., sequence modification polynucleotides) 514F, 514R, 520F and 521F, “T” droplets appeared after having been targeted and edited by this DLR molecule in combination with each correcting template, demonstrating successful C→T genetic conversion at codon 158 site of human ApoE gene.

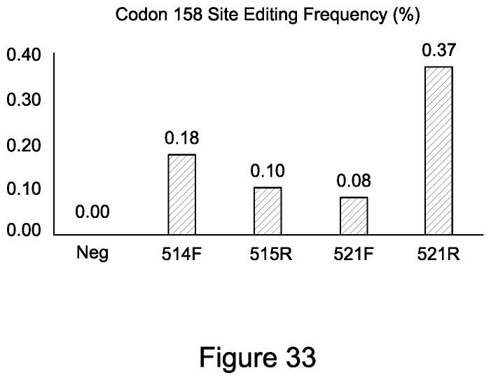

FIG. 33 shows C→T gene conversion frequencies as measured by ddPCR after DLR-based gene editing. Panel A shows absolute counts of individual droplet event per channel for untargeted (control) and targeted conditions. Codon 158 editing frequencies (defined as cellular T to C conversion percentages), was determined by calculating percentages of T droplet events divided by their sum of C and T droplet events. DLR-based gene editing frequencies ranged from 0.08% (when using sequence modification polynucleotide 520F) to 0.37% (when using sequence modification polynucleotide 520R) in comparison to untargeted HEK293 negative control with 0.00% background conversion. These results further demonstrate and confirm that DLR-based gene editing has potential to repair genetic mutations that are clinically relevant to development of therapies for genetic diseases and to do so in a way that is safer than technologies that require induction of genetic breakages to create genetic modifications.

### Example 5: Editing an Endogenous Genetic Target in a Second Cell Type

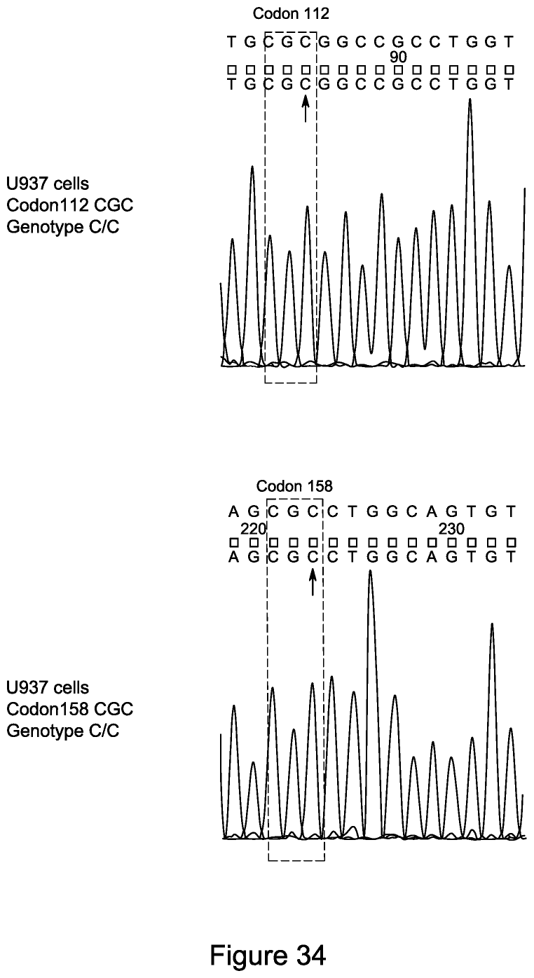

In this example human U937 cell line was used to demonstrate use of a DLR-based editing system in another type of mammalian cell. U937 cells are Human histolytic lymphoma cells and have a genotype of ApoE4/E4, which results in having Arginine at both codon 112 and 158. Arginine is encoded by CGC. FIG. 34 shows an E4/E4 genotype of U937 by Sanger Sequencing, demonstrating CGC at both codons 112 and 158. In a previous example with cell line HEK293, which had genotype apoE3/E3, a T-to-C conversion at codon 112 was illustrated. Reported herein, this example discloses that a C-to-T conversion at codon 112 could be achieved, in addition to the usage of a different cell line.

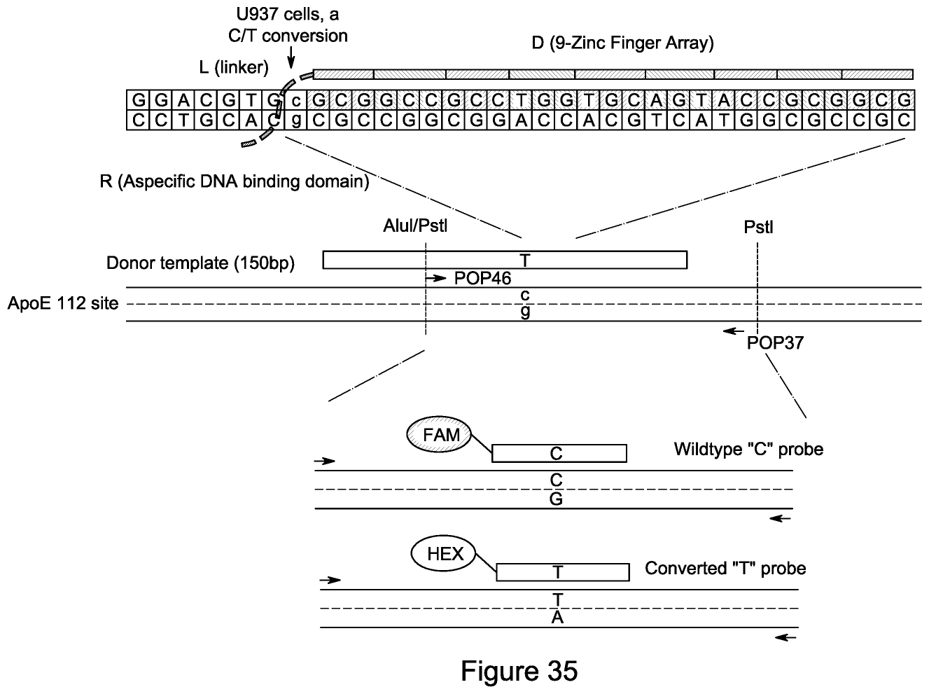

FIG. 35 illustrates an approach taken for this example. This example was aimed at gene editing of an endogenous genomic target around codon 112 of the human ApoE gene in U937 cells. In this example, a DLR molecule, encoded on plasmid pb6 (SEQ ID NO.: 21) encompassed as a DNA recognition domain an array of 9 zinc fingers, was specifically designed to recognize a 27-nucleotide sequence of 5′-GCGGCCGCCTGGTGCAGTACCGCGGCG-3′ (SEQ ID NO.: 8) on the leading strand of human ApoE. A targeted nucleotide “C” is displayed as lower case letter “c” 5′ upstream of a binding site. In this embodiment, an R element was designed to bind to the opposite strand, in this case the lagging strand, in a non-sequence-specific manner. In this embodiment, an ssODN donor template (i.e., sequence modification polynucleotide) with a sequence of 5′-CCCCGGTGGCGGAGGAGACGCGGGCACGGCTGTCCAAGGAGCTGCAGGCGGCGCA GGCCCGGCTGGGCGCGGACATGGAGGACGTGTGCGGCCGCCTGGTGCAGTACCGCG GCGAGGTGCAGGCCATGCTCGGCCAGAGCACCGAGGAGC-3′ (SEQ ID NO.: 33) was used. This was a 150-nucleotide DNA oligonucleotide with a desired C-to-T (bold and underlined) substitution roughly located in the middle of this oligonucleotide. A relative position of a correction ssODN (i.e., sequence modification polynucleotide) and binding positions of a common primer pair POP46 (SEQ ID NO.: 24) and POP37 (SEQ ID NO.: 80) are also indicated in FIG. 35. A common primer POP46 locates inside this ssODN template (i.e., sequence modification polynucleotide), while POP37 resides outside. Allele-specific probes conjugated with fluorophores FAM and HEX were designed to distinguish between “C” and “T” respectively. PstI restriction enzyme sites indicated could be used in preparations for ddPCR reactions.

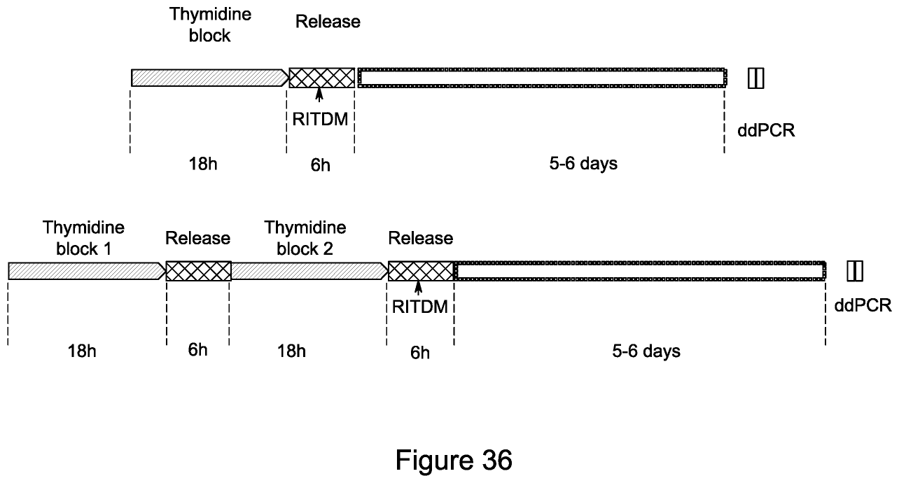

In this example, U937 cells were subjected to either one thymidine block or double blocks prior to introduction of plasmid pb6 (SEQ ID NO.: 21) and a 150-nucleotide correction template (SEQ ID NO.: 33) by electroporation, shown in FIG. 36. Application of thymidine treatment was done to synchronize U937 cells to a specific point in their cell cycle, this to enhance editing frequencies.

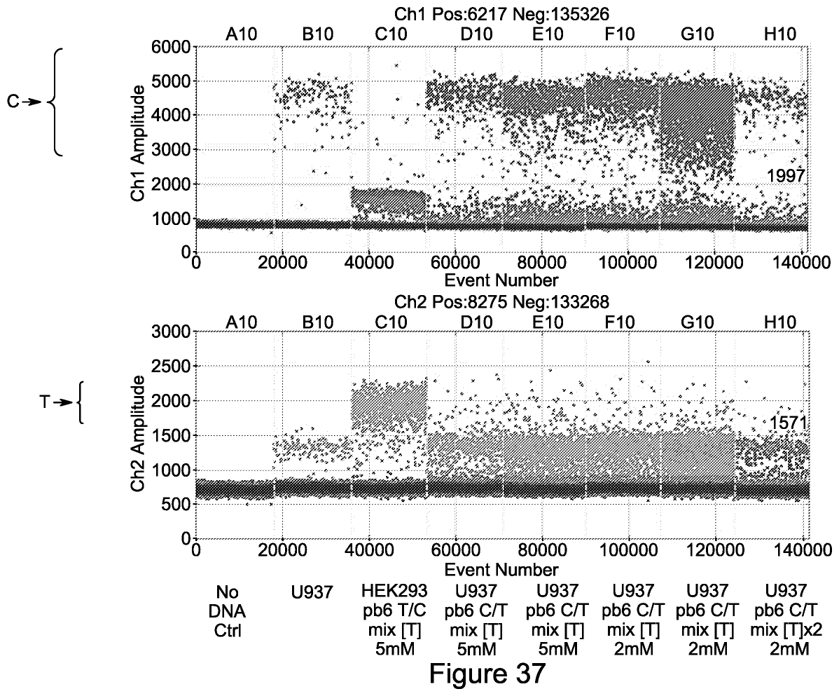

FIG. 37 demonstrates successful C→T genetic conversion at codon 112 of human ApoE as measured by ddPCR. In this example, after transfection, U937 cells were allowed to recover and grow on complete RPMI 1640 medium with 10% FBS for seven days. After seven days genomic DNA was isolated and used in digital droplet PCR analysis to determine nucleotide “C” or “T” at codon 112 of ApoE. Raw droplet data is shown in FIG. 37 where “C” droplets are displayed in the top panel, while “T” droplets are displayed in the lower panel. Lane A10 represents no DNA input as negative control, showing neither “C” nor “T” droplets. Lane B10, representing untargeted U 937 cells (homozygous C/C), showed only “C” droplets. Lane C10 shows HEK 293 cells previously targeted by pb6 as a positive control (heterozygous T/C genotype), showed both “C and “T” droplets. Lanes D10 and E10 represent results with U937 cells, using a single 5 mM thymidine block; Lane F10 and G10 are U937 using a single 2 mM thymidine block; Lane H10 corresponds to U937 using a double 2 mM thymidine block. After U937 was transfected with pb6 and ssODN donor template (i.e., sequence modification polynucleotide), “T” droplets appeared under all experimental conditions. This experiment shows that after being targeted and edited by this DLR molecule, in combination with any of the provided correction templates, successful C→T genetic conversion at codon 112 of human ApoE occurred.

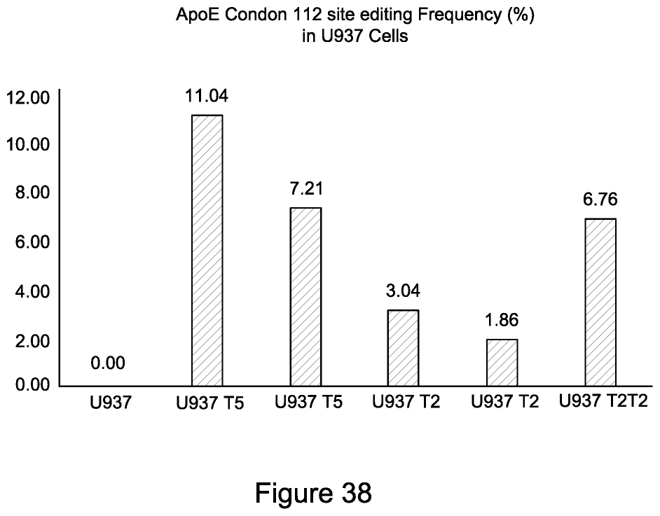

FIG. 38 shows C→T gene conversion frequencies measured by ddPCR after this DLR-based gene editing. Panel A shows absolute counts of individual droplet events per channel for untargeted (control) and targeted cells. Codon 112 editing frequencies, which were cellular C→T conversion percentages, were defined as percentage of T droplet events divided by the sum of C and T droplet events. Conversion rates in U937 were higher than conversion rate observed in HEK293. Potential underlying reasons for this difference may have been that a conversion from C→T may have been more favorable in this experimental setting compared to a T→C conversion, or that U937 having a lower copy number of chromosome 19 compared to HEK293, may have made ddPCR detection easier, or there may have been different cell intrinsic differences or other reasons. What is important for this disclosure is that conversion could be achieved in multiple cell lines.

### Example 6: DLR Designs: Generation and Evaluation of Various R Elements

An aspect of this disclosure is that various elements of a DLR molecule can be modular in design. In this example, a variety of non-cleaving (i.e., no cleavage activity), modular R elements were designed and evaluated for their functionality within one or more functional DLR molecules. Gene editing activity of these DLR molecules was characterized.

FIG. 39 illustrates generation of a number of different R-elements as parts of functional DLR molecules. For example, a type of R element was designed based on a core fold present in certain PD-(D/E)xK structures (Steczkiewicz, Muszewska, Knizewski, Rychlewski and Ginalski, 2012, Nucleic Acids Res 40 7016-7045, which is herein incorporated by reference in its entirety) identified in a large and highly diverse protein superfamily involved in nucleic acid maintenance, such as, BtsI or FokI. This core architecture is highly conserved, consisting of three antiparallel beta-sheets connected by two loops, referred as sheet-loop-sheet-loop-sheet fold. Antiparallel beta-sheets have been known to have, in general, high thermo-dynamic stability In FIG. 39, three beta-sheets and two loops, secondary structural elements of conserved core folds from BtsI and FokI, were aligned. Active site residues involved in DNA cleavage activity were aspartic acid (D) in beta-sheet 2 and aspartic acid (D) or Glutamic acid (E) in beta-sheet 3, and they were highlighted in black blocks. In this example, a newly created R element core (SEQ ID NO.81) for usage in DLR molecules was created by combining BtsI's 3 beta-sheet and loop2 with FokI's loop1, in combination with a number of amino acid changes done to obtain a stable and functional core. Active residues D or D/E were mutated to abolish nuclease activity, while retaining non-sequence-specific DNA binding ability. Moreover, these R elements were linked to a D element through a short linker comprising of amino acids LRGS, (SEQ ID NO. 1), where its D element was a 9-zinc finger array that recognized a 27-nucleotide DNA (SEQ ID NO.: 8) sequence close to codon 112 of human ApoE. In addition a wider set of R elements was generated by creating a series of active site residue mutations. That is, a given point mutation was introduced into an R element and, importantly, the R element could maintain its functionality in the presence of that point mutation. This process was repeated for various point mutations. This demonstrates that an R element can function in a non-sequence specific manner and can maintain functionality even if one or more point mutations is introduced into a given R element. This was done to deactivate potential nuclease enzymatic activity by site directed mutagenesis. These constructs were labeled pb1 through pb12 (SEQ ID NOS.: 34-44), and pb16 and pb17 (SEQ ID NOS.: 45 and 46). In particular, a PD active site residue was mutated to PA (pb16) and PN (pb17), respectively. In native FokI, either of these mutations abolished enzymatic activity, or at least reduced activity with orders of magnitude (Bitinaite, et al, 1998, Proc Natl Acad Sci USA 95 10570-10575; Wah, et al, 1998, Proc Natl Acad Sci USA 95 10564-10569, each of which is herein incorporated by reference in its entirety). For (D/E), active site residues mutations were created replacing it with Q (pb1), N (pb2), S (pb3), T (pb4) A (pb5), V (pb6) L (pb7), I (pb8), H (pb9), R (pb10), K (pb11), and M (pb12), respectively.

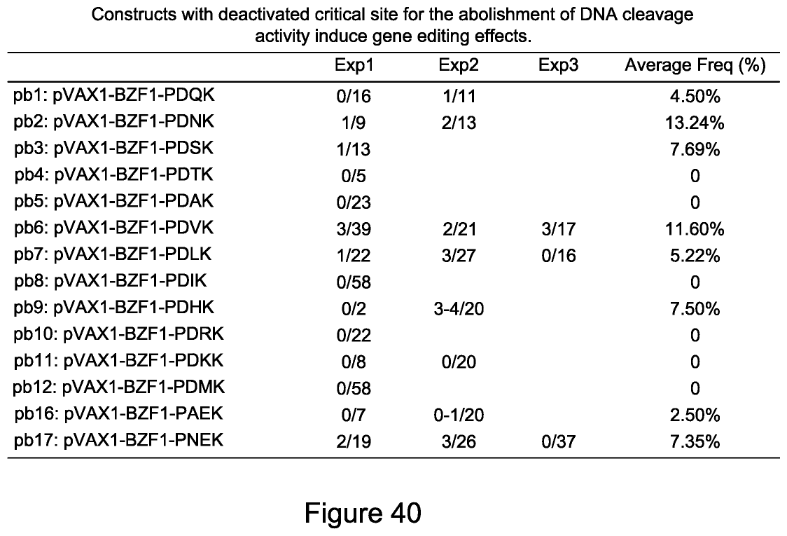

FIG. 40 shows the characterization of gene editing activities of these constructs with various R elements. In this example, various R elements were fused with a D domain through an LRGS linker (SEQ ID NO. 1), creating DLR molecules designed to be used for gene editing codon 112 of human ApoE. Using a same method as illustrated in FIG. 16, DLR molecules as described herein was delivered into HEK293 cells together with an ssODN donor template (i.e., sequence modification polynucleotide). A ddPCR assay was employed to identify positive single cell clones that had a genetic T→C conversion at ApoE codon 112. Remarkably, both “PD” mutants, pb16 and pb17, gave rise to positive clones with an average editing frequency of 2.5% and 7.35% respectively. Similarly, 6 out of 12 mutants of active site residue (D/E), pb1, pb2, pb3, pb6, pb7 and pb9 produced gene-converted clones with an average frequency ranging from 4.5% to 13.24%. These results provide several examples of functional DLR molecules, each having a variation in an R element.

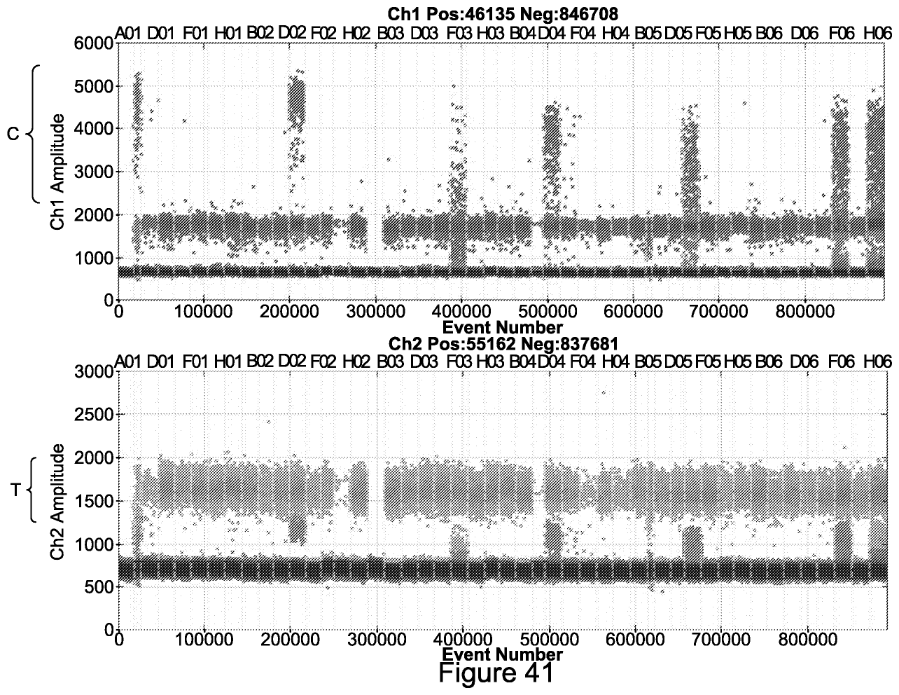

FIG. 41 shows representative results of ddPCR analysis as used for identification of positive clones that contained a T-to-C conversion at codon 112 of human ApoE in HEK293 cells, obtained when using R elements with various mutations of active side residues. Together, these results also demonstrate that DLR-based gene editing does not depend on catalytic activity involving PD-(D/E)XK associated phosphodiesterase activity. These results support that in using a DLR molecule, a combination of non-sequence specific DNA binding activity (by its R-domain) with sequence-specific DNA binding provided by its D-domain may provide advantages not achieved by other gene editing systems or approaches.

To further exemplify the modularity of R-elements, further variations were designed and evaluated. Catalytically inactivated PD-(D/E)XK cores were artificially diversified by interchanging segments of sheet-loop-sheet-loop-sheet folds from different PD-(D/E)XK sources.

FIG. 42 shows exemplary R elements with variable PD-(D/E)XK cores. Panel A shows an amino acid sequence alignment from two functionally designed D elements (pb6 and pb17), which were aligned to core amino acid sequences of a number of naturally occurring PD-(D/E)XK nucleases. Critical residues involved in DNA cleavage were highlighted. Aspartic acid (D) in beta-sheet 2 from various nucleases aligned with either “D” in pb6 or mutated alanine (A) in pb17. Similarly, either aspartic acid (D) or glutamic acid (E) in beta-sheet 3 aligned with mutant valine (V) in pb6 or “E” in pb17. Therefore amino acid sequences of beta sheet1-loop1-beta sheet2-loop2-betasheet3 fold could be aligned as displayed in Panel A. In order to demonstrate that design of a PD-(D/E)XK core fold could be essentially modular, Panel B shows constructs that were made in which a beta sheet 2-loop 2-beta sheet 3 sequence was replaced by an equivalent sequence from FokI (pb18, SEQ ID NO.47), EcoRV (pb19, SEQ ID NO.48), SstI (pb20, SEQ ID NO.49), MvaI296 (pb21, SEQ ID NO.50), EAB43712 (pb22, SEQ ID NO.51), BsmI (pb23 SEQ ID NO.52), BsrDI (pb24, SEQ ID NO.53), and BtsI (pb25, SEQ ID NO.54), respectively. The active residues, E or D in beta sheet 3, were deactivated and replaced by V to abolish any nuclease activity. Similarly, Panel C demonstrates that a loop 1 structure was essentially exchangeable for equivalent structures to create versions in which loop 1 of construct pb17 was replaced by a similar loop 1 from BtsI (pb26, SEQ ID NO.: 55), SstI (pb27, SEQ ID NO.: 56), Mva1296 (pb28, SEQ ID NO.: 57) EAB43712 (pb29, SEQ ID NO.: 58), BsmI (pb30, SEQ ID NO.: 59), and BsrD1-A (pb31, SEQ ID NO.: 60) respectively. Active residue, D in beta sheet 2, was inactivated and replaced by A to abolish nuclease activity.

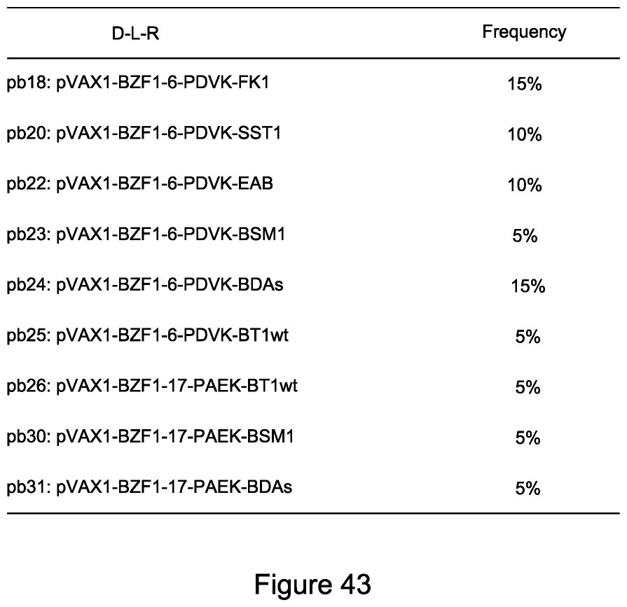

FIG. 43 shows characterization of gene editing activities of these constructs with various variable PD-(D/E)XK cores in their R elements. In this example, these various R elements were fused with D domain through an LRGS linker (SEQ ID NO. 1), enabling these DLR molecules to recognize and target codon 112 of human ApoE. Using the same method illustrated in FIG. 16, each DLR molecule was delivered into HEK293 cells with an ssODN donor template (i.e., sequence modification polynucleotide). A ddPCR assay was employed to identify positive single cell clone having a genetic T→C conversion at ApoE codon 112 in HEK293 cells. Genomic DNA from single cell clones was employed to identify positive single cell clones having genetic T→C conversions at ApoE codon 112 in HEK293 cells. Only constructs yielding positive results are displayed.

Surprisingly, 6 out of 8 constructs in which a beta 2-loop 2-beta 3 structure was replaced were functionally active in gene editing. This provides a clear indication that this element of design is highly modular and provides great flexibility for use in achieving genetic modifications. This approach can be extended to a variety of structures and designs.

For the loop 1 structure, 3 out of 6 structures were functional. This finding also supports modularity of this type of element that can be extended to a variety of structures and designs. Since this element would have been expected to interact with a DNA backbone and/or major/minor groove, it was very surprising that a high proportion of variants were actually active.

Taken together, this example illustrates that design of an R element can be extremely diversified. In this example a wide series of R elements were shown to be functionally active and that many variations could be made using a PD-(D/E)XP core type fold. The embodiment herein provides exemplary functional DLR molecules and demonstrates modularity of design, with a potential for wider choices in DLR molecule designs offering maximum flexibility providing technologies for successful gene editing applications across a variety of situations.

### Example 7: DLR Designs: Generation and Evaluation of Catalytically Inactive Cas9 as D-Domain

In this example another type of sequence-specific DNA binding motif as D element was examined to further illustrate versatility of this disclosure. A DLR molecule was designed that made use of a Cas9 protein as a D element. In this example a zinc finger array was replaced by a catalytically inactive Cas9 domain.

The clustered regularly interspaced short palindromic repeat (CRISPR) system is a prokaryotic adaptive immune system that has been adapted for genome engineering in a variety of organisms and cell lines. CRISPR/Cas9 protein-RNA complexes localize a target DNA sequence through base pairing with a guide RNA, creating a DNA double stranded break at a locus specified by its guide RNA. Catalytically “dead” Cas9 (dCas9), which contains Asp10Ala (D10A) and His840Ala (H840A) mutations that inactivate its nuclease activity, retains its ability to bind to DNA in a guide RNA-programmed manner but does not cleave DNA backbone (Guilinger, et al., 2014, Nat Biotechnol 32 577-582, which is herein incorporated by reference in its entirety). This example demonstrates that conjugation of dCAS9 with an R element via a linker enables DNA editing without intentionally introducing a DNA breakage, e.g., at or near a target site.

FIG. 44 is a schematic depicting an engineered DLR molecule that comprises a catalytically inactive Cas9 (dCas9). It also illustrates its characterization in gene targeting and editing. dCas9 can be used as a D and/or R element in a DLR molecule. As a D element dCas9 is sequence-specific; where dCas9 is used as an R element it may be used, for instance, in combination with a D element comprising a sequence-specific binding unit such as a zinc finger array, TALE, a second dCas9, etc.

FIG. 44, panel A illustrates targeting and editing at an EGFPDP2 gene by this dCas9-L-R chimera construct. An EGFPDP2 rescue reporter system was used to detect gene conversion after transfection with this newly designed fusion protein, donor template and guide RNA designed for this Cas9-based D-L-R system. As DNA recognition domain in this DLR example an inactivated cas9 (dCas9) is used, which had double point mutations D10A and H840A to abolish its catalytic ability to create double stranded DNA breaks. Typically, Cas9 mediated genome editing involves cleavage of double-stranded DNA at a sequence programmed by a short, single-guide RNA. In this example a synthesized guide RNA, POP45-crRNA, 5′-mG*mA*GCUGGACGGGGACGUAAAGUUUUAGAGCUAUG*mC*mU-3′ (SEQ ID NO.: 61), annealed with TracrRNA (Genscript, Piscataway, NJ) was designed to target a sequence 5′-GGAGCTGGACGGGGACGTAAACGG-3′ (SEQ ID NO.: 62) in EGFPDP2. Panel B is a molecular map of this D(dCas9)LR (SEQ ID NO.: 64) chimera construct used in this example, in which dCas9 is fused by an amino acid linker to an R element, under the control of a CMV promoter. Its corresponding translated amino acid sequence (SEQ ID NO.: 63) is in Table 1.

For this DLR molecule, at its N-terminus, a 3×FLAG epitope and a nuclear localization signal were built-in, followed by a dCas9 module fused by a linker to an R element. A linker was specially designed for this example to be longer than a linker used in previous examples that used zinc finger arrays, due to considerations of a much larger size of this dCAS9 protein compared to zinc finger arrays. A linker sequence was used in this example that comprises of amino acids LRQKDAARGS (SEQ ID NO.: 65). This linker was designed to enable a geometric ability to allow this specific DLR molecule to bind to both strands of DNA.

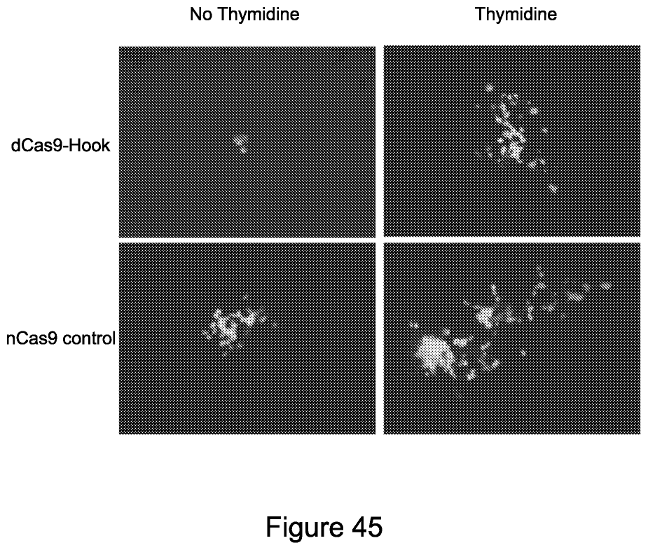

FIG. 45 shows successful restoration of functional EGFP expression by dCas9-L-R mediated gene editing. EGDPDP2 HEK293 cells were electroporated with a plasmid encoding dCas9-L-R, guide RNA, and a single strand DNA oligonucleotide donor template. This cell reporter system allowed for detection of gene conversion as was detected by cells turning fluorescent. Two weeks post transfection, both under conditions using or not using thymidine for synchronization, cells using dCas9-DLR turned green. As a positive control, a version of Cas9 was used that contains a single point mutation (D10A), which converts Cas9 into a nicking endonuclease, enabling genetic conversion by inducing single-stranded DNA nicks.

Since dCas9 could be used as sequence specific D element in a DLR gene editing system (i.e., a RITDM system), it was another clear indication of versatility of DLR molecules for gene editing. It also emphasized the potential to use multiple types of DNA binding domains. This versatility suggested that other DNA sequence specific binding domains could also be used as parts of DLR molecules.

### Example 8: DLR Designs-Design of DLR with a Sequence-Specific R Element

To further illustrate use of DLR molecules, and the versatility of DLR molecule technology and performance, a DLR molecule was designed that made use of a zinc finger array as an R element. As has been described herein, in contrast to many other gene editing systems, DLR-based DNA editing systems do not depend on creation of double-or single strand DNA breaks to induce gene conversion. A DLR molecule comprising zinc finger arrays in both R and D elements provides additional support that technologies provided by this disclosure and exemplified herein do not depend on induction of DNA backbone cleavages mediated by nuclease or nickase activity by a DLR molecule itself.

FIG. 46 illustrates a schematic depicting a DLR molecule comprising of DNA sequence-specific binding elements at both N- and C-terminus, with a linker in the middle.

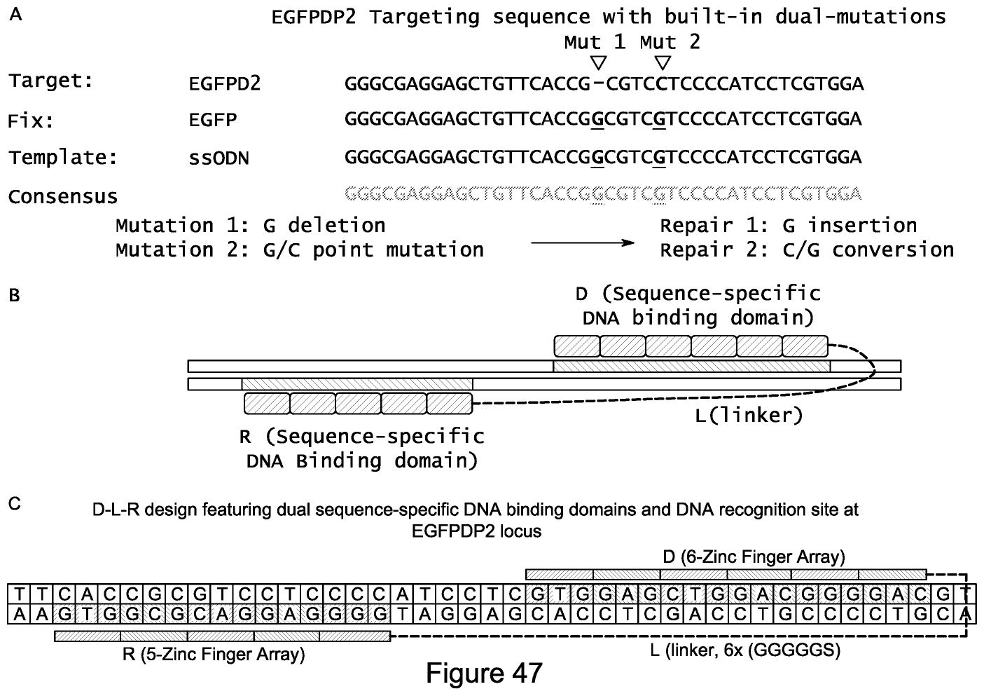

As provided herein, gene targeting and editing can be induced by providing one DNA binding domain binding to a leading strand and another DNA binding domain binding on a lagging of the same DNA molecule, at or close to a target site. In order to demonstrate that such a DLR molecule could be used for gene conversion, a reporter system based on an Enhanced Green Fluorescent Gene (EGFP), as described throughout these Examples, was used (see FIG. 9). FIG. 47 shows a schematic approach to targeting and editing EGFPDP2 mutant genes by using a DLR molecule that comprises two zinc finger arrays (as D-domain and as R-domain). Panel A illustrates molecular details of core elements of this specific gene conversion using the RITDM e system described in this Example. An EGFPDP2 targeting and repairing strategy was based on EGFPDP2 containing two mutations: a deletion of nucleotide G and a G→C point mutation. A donor template was designed to both insert a G and convert C to G at these two mutation sites of EGFPDP2. Successful EGFP gene repair would restore in-frame expression of EGFP. Panel B illustrates interaction between DLR with dual non-cleavage zinc finger arrays and double stranded DNA at this target site in a genome. Both DNA binding elements were designed to recognize and bind with DNA in a sequence-specific manner, each on a different DNA strand. Panel C shows these dual zinc arrays binding two recognized sites of a EGFDP2 mutant locus on each strand of DNA.

Plasmid pb42 (SEQ ID NO.: 66) encoded this specific DLR construct, which contained two DNA sequence specific binding elements and one linker. In this embodiment, coding sequences of this DLR (SEQ ID NO.: 67) were cloned into plasmid vector pVAX1 (ThermoFisher, Waltham, MA) using HindIII and NotI from 5′ to 3′, thus expressing this DLR (SEQ ID NO.68) with a Flag-tag and a Nuclear Localization Signal (NLS) at its N-terminus under control of a CMV promoter. This D element was a 5-zinc finger array, designed to recognize a strand of DNA with sequence 5′-GGGGAGGACGCGGTG-3′ (SEQ ID NO.: 4). In this example, a longer linker element with amino acid sequence

GGGGGSGGGGGSGGGGGSGGGGGSGGGGGSGGGGGS or 6 repeats of GGGGGS (SEQ ID NO.: 69) was used. In this Example, an R-element with a 6-zinc finger array was used, designed to recognize an opposite strand of DNA with sequence 5′-GTGGAGCTGGACGGGGAC-3′ (SEQ ID NO.: 6). This R element was designed as a sequence-specific domain and the amino acid sequence of this protein encoded on plasmid pb42 (SEQ ID NO.68) is listed in Table 1.

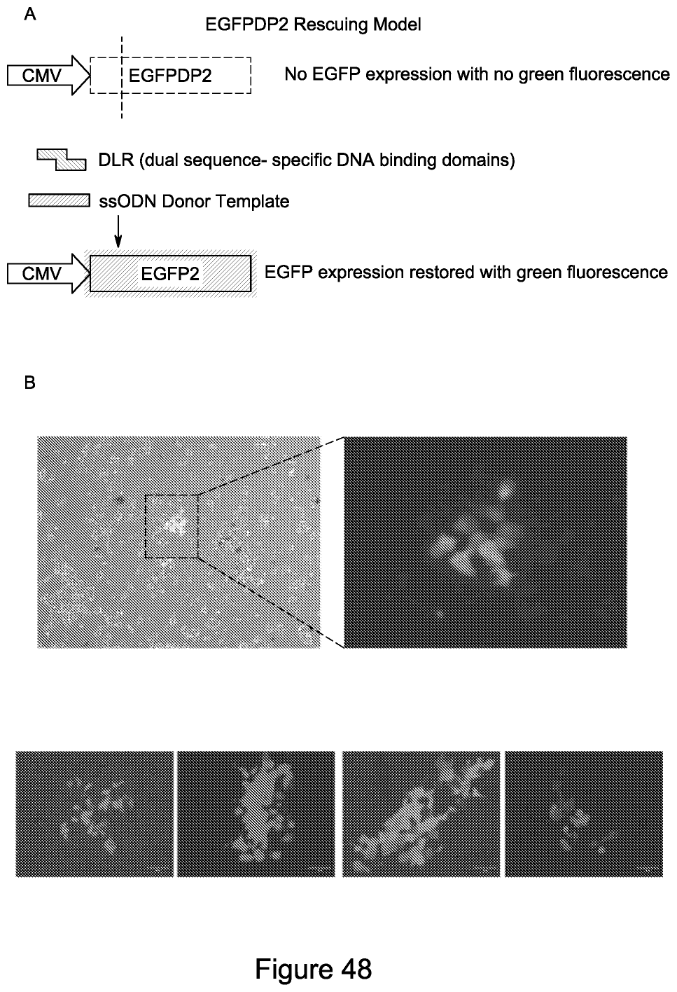

FIG. 48 demonstrates that EGFPDP2 was successfully targeted and repaired by a non-cleavage DLR molecule with double zinc finger arrays. Panel A is a schematic illustrating a testing model of genetic EGFPDP2→EGFP conversion by this DLR with dual zinc finger arrays. HEK293E GFPDP2 reporter cells were transfected with plasmid pb42, along with a 142-nucleotide in length ssODN correction template (i.e., sequence modification polynucleotide; SEQ ID NO.70) by electroporation. Panel B demonstrates that mutant EGFPDP2 was repaired and expressed functional EGFP. Seven days after transfection, multiple individual green cells and green cells clusters appeared when observing with a green fluorescence inverted microscope. After several passages, green cells were still observed. These results demonstrate that mutant EGFPDP2 was genetically repaired and EGFP protein expression was restored, confirming that gene conversions in these cells were achieved and lasting, as they propagated through passaged cells.

### Example 9: DLR and DNA Replication Fork Interaction

In order to demonstrate a direct interaction between DLR molecules with components of a replication fork, analyses were done that made use of an in situ Interaction at Replication Fork (“SIRF”) methodology (Roy et al., 2018, Journal of Cell Biology, 217 1521-1536, which is herein incorporated by reference in its entirety). In SIRF, newly synthesized DNA at replication forks was labeled with EdU and then biotinylated by click chemistry between EdU and biotin-azide. Cells were subsequently incubated with primary antibodies against biotin and a protein of interest. Then, cells were incubated with secondary antibodies conjugated with oligonucleotides that functioned as proximity probes. If secondary antibodies were in a proximity of <40 nm and indicative of direct interaction between an examined protein and biotinylated DNA, DNA oligomers would be able to anneal, guiding formation of a nicked circular DNA molecule. After ligation, DNA circles could then serve as templates for localized rolling circle amplification. DNA sequence-specific fluorescent DNA probes would then anneal to amplified DNA circles, allowing a signal to be visualized and quantified.

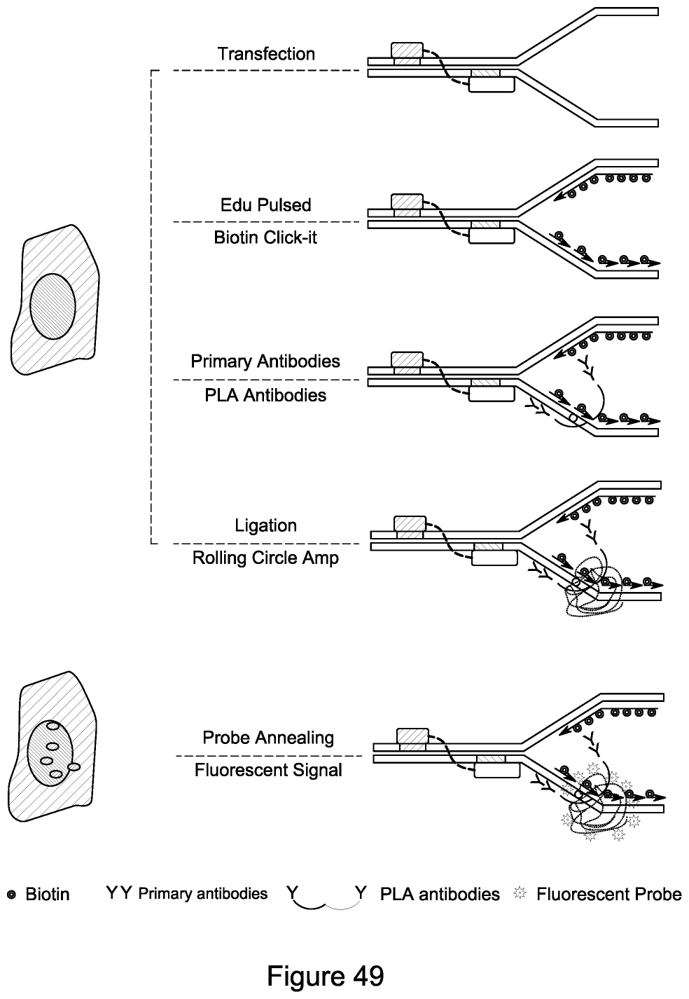

FIG. 49 illustrates a schematic representation outlining in situ analysis of protein interactions at DNA replication fork. In this example, a SIRF assay was performed to demonstrate direct association of a DLR molecule with EdU-labeled nascent DNA at replication forks. HEK293 cells were transfected with a Flag-tagged DLR molecule, grown in microchamber-slides and pulsed with 100 μM EdU for 8 minutes, followed by EdU biotinylation using click chemistry. Cells were incubated with primary antibodies overnight at 4° C. (1:250 rabbit anti-biotin antibody with 1:1000 mouse anti-Flag M2 antibody). Cells were washed twice with PBS and incubated with pre-mixed Duolink PLA plus and minus probes for 1 h at 37° C. Subsequent steps in proximal ligation assay were carried out using a Duolink PLA Fluorescence Kit (Millipore Sigma, Burlington, MA) according to the manufacturer's instructions. Slides were stained with DAPI (4′,6-diamidino-2-phenylindole) and imaged by an upright fluorescent microscope. Detection of fluorescent puncta demonstrated direct interaction and association between active replication forks and DLR molecules.

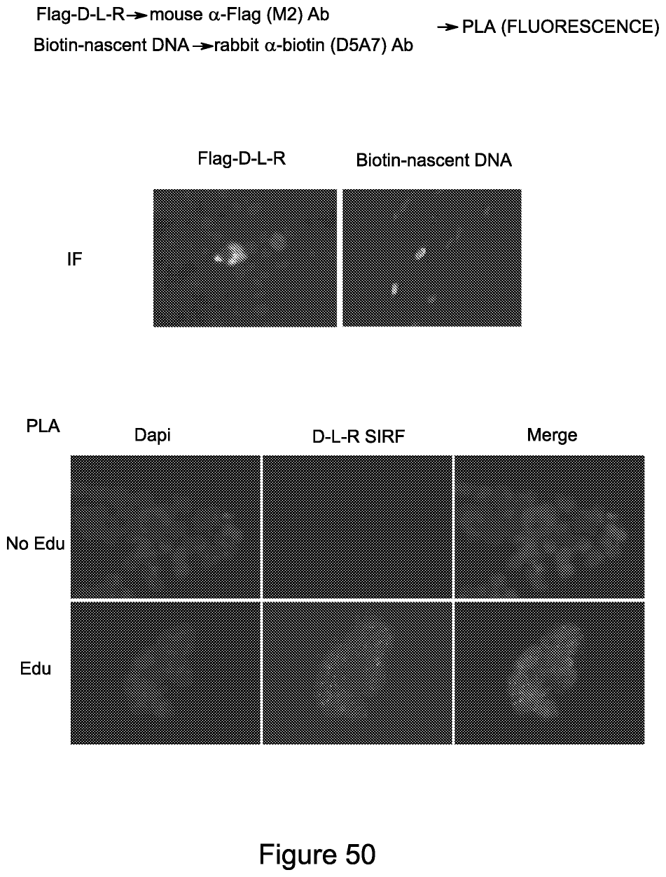

FIG. 50 shows close proximity between a DLR molecule and a replication fork. Immunofluorescent staining showed expression of a DLR molecule in transfected HEK293 cells. Nascent DNA representing replication forks were biotin labeled and detected by an anti-biotin antibody. A “no-Edu pulse” experiment was used as a negative control for SIRF, as no red fluorescent puncta could be detected. In presence of Edu, DLR-SIRF signals were detected. Red fluorescent puncta could clearly be detected in transfected cells. Representative images of SIRF signals demonstrating a direct interaction between DLR molecules and replication forks are shown in FIG. 50.

This example demonstrates that a DLR molecule can interact with a DNA replication fork and provide an opportunity for a correction oligonucleotide to anneal to a complementary, single-stranded DNA sequence that was (temporarily) exposed when a replication fork was blocked from progressing. DLR binding could interfere with progression of a replication fork at a binding site, and so it could prolong exposure of a single stranded DNA conversion site, thus triggering gene targeting and editing that is not dependent on introducing DNA breaks.

### Example 10: RITDM-Mediated Gene Editing Efficiency Responds to Various Factors Associate with Replication Fork and Mismatch Repair Pathway

In this example experiments were conducted to determine if reduction of specific factors involved in various DNA repair processes could influence DNA conversion rates. Ability to influence DNA conversion rates provides advantages for use in conjunction with a DLR molecule. For this evaluation, conversion at codon 112 of human ApoE was used.

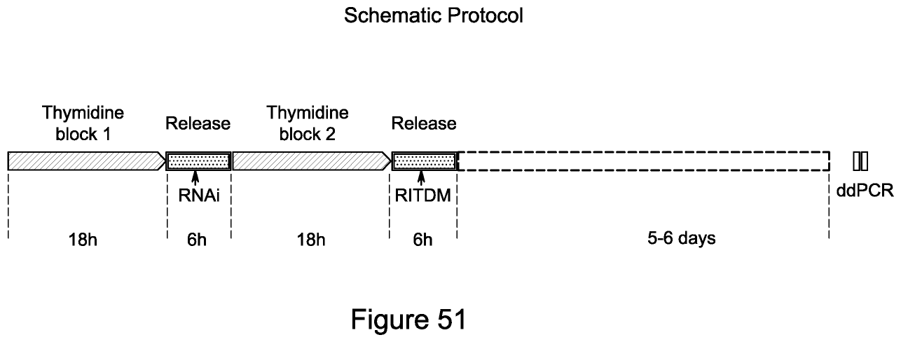

FIG. 51 illustrates experimental schematics of a timed delivery of a DLR molecule as well as RNAi with cell cycle synchronization in HEK293 cells for genome editing. Cell cycle synchronization was chemically achieved by using a double thymidine “block” approach as illustrated in FIG. 51. Each “block” lasts approximately 18 hours after addition of 5 mM thymidine to cell culture medium, in this example, containing 15% FBS in DMEM. After a first thymidine block, a siRNA molecule (50 μmol working concentration) was introduced into cells by using a Lipofectamine RNAiMax reagent to inhibit gene expression or translation, thereby reducing certain factors relevant to processes of DNA replication or DNA repair. After a second thymidine block, cells were released into a normal medium followed by electroporation of a DLR molecule-encoding plasmid, pb6, and an ssODN correction template (i.e., sequence modification polynucleotide) specific for ApoE codon 112 conversion. Methods of detection of genetic T→C conversion as used in this example have been elaborated on previously in Example 2. Five days post gene editing by DLR, genomic DNA were extracted and genetic T→C conversion of this target gene was measured by ddPCR. Gene editing frequencies were calculated using an algorithm described in Example 2.

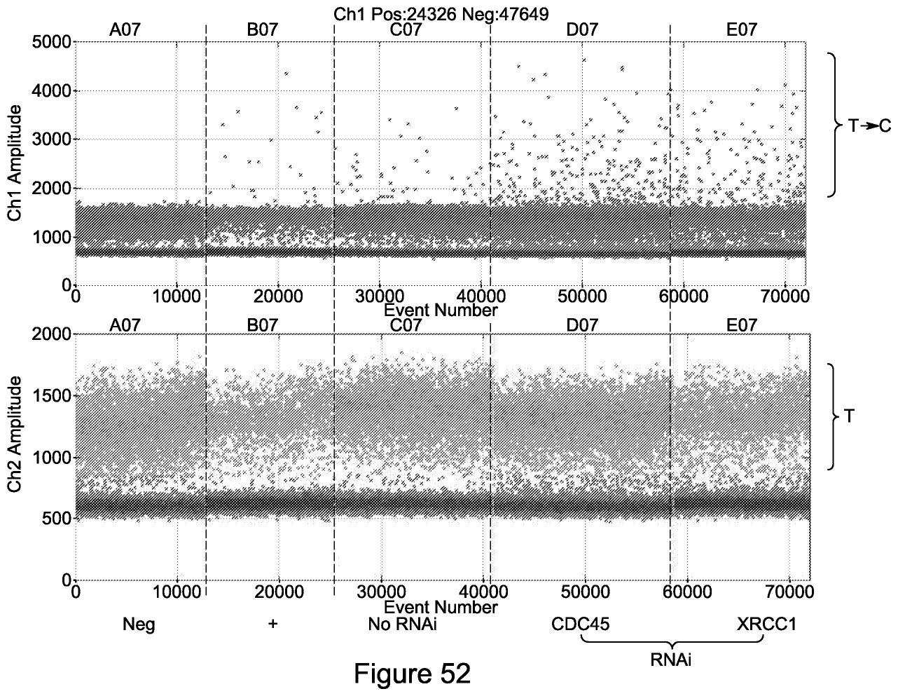

FIG. 52 shows representative results from impacts on gene editing efficiency by reduction of Cdc45 or XRCC1 by RNAi (here, siRNA was used). No DNA input was used as negative control, showing neither “C” nor “T” droplets. A pool of previously edited HEK293 cells was used as a positive control, since these had a heterozygous T/C genotype at codon 112 of human ApoE, hence they showed both “C” and “T” droplets. In this example, no siRNA addition was used as a background reference. Addition of siRNA to inhibit either Cdc45 or XRCC1 showed more “C” droplets compared to a no siRNA addition reference background, demonstrating that reduction of Cdc45 or XRCC1 enhanced DLR-based gene editing efficiencies.

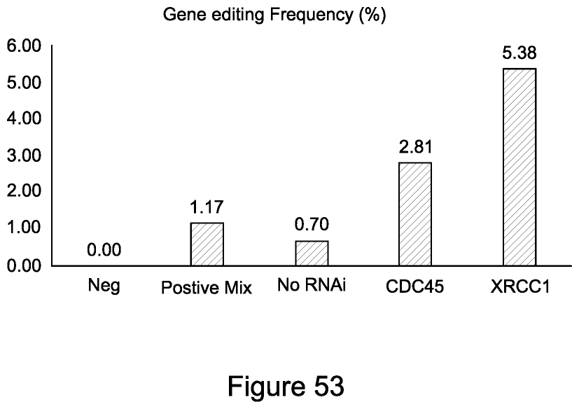

FIG. 53 shows T→C gene conversion frequencies measured by ddPCR after DLR-based gene editing. Editing frequencies were expressed as cellular T to C conversion percentages, defined as percentage of C droplet events divided by the sum of C and T droplet events. Inhibition of Cdc45 increased gene editing frequencies by about 4-fold when compared to no RNAi addition; while inhibition of XRCC1 achieved an approximately 8-fold increase in frequency.

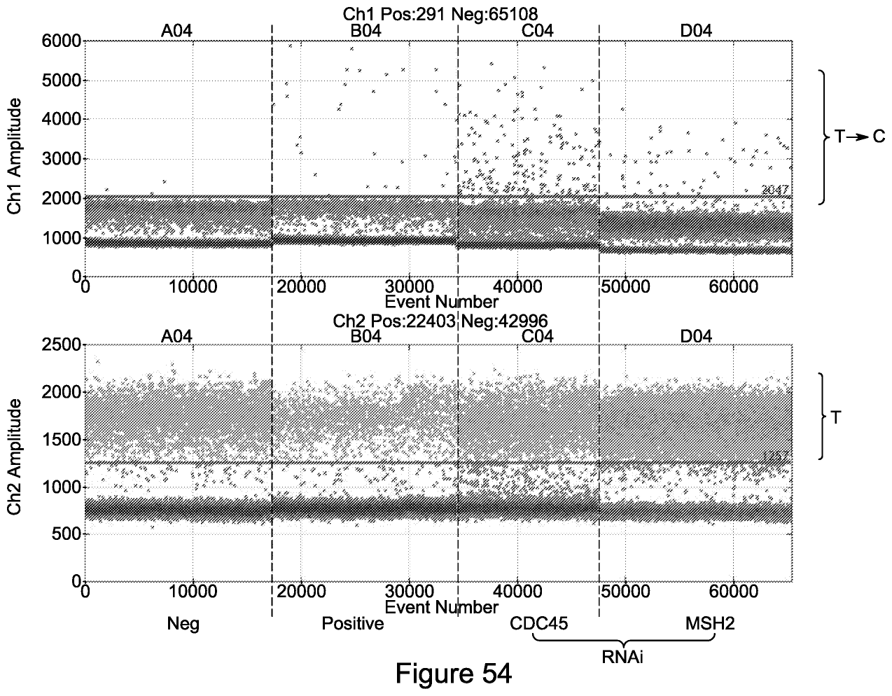

FIG. 54 shows representative results from impacts on gene editing efficiency by reduction of Cdc45 or MSH2 by RNAi (here, siRNA was used). No DNA input was used as a negative control and a pool of previously edited HEK293 cells was used as a positive control (heterozygous T/C genotype at codon 112 of human ApoE), showed both “C” and “T” droplets. In this example, effects on gene editing efficiencies were compared when inhibiting Cdc45 and MSH2. Addition of RNAi of Cdc45 showed more “C” droplets compared to a reference background. However, inhibition of MSH2 showed fewer “C”, droplets representing a decrease in efficiency of DLR-based gene editing.

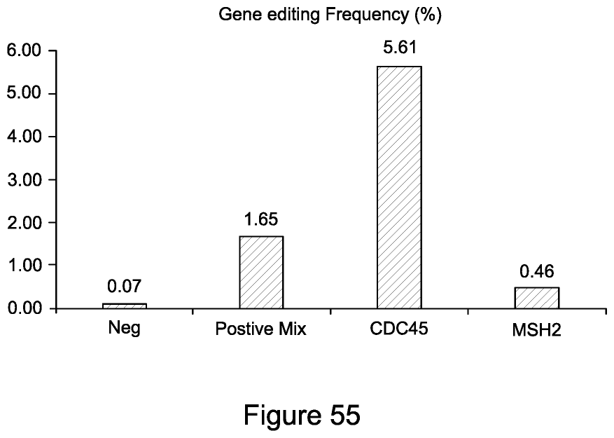

FIG. 55 shows T→C gene conversion frequencies measured by ddPCR after DLR-based gene editing. Editing frequencies are calculated using a same algorithm as shown in FIG. 53. Inhibition of Cdc45 achieved about a 4-fold increase in gene editing frequencies, while reduction of MSH2 decreased gene editing frequencies by about 4-fold.

In eukaryotic cells, Cdc45 is an essential protein involving initiation of DNA replication. As a member of the eukaryotic replicative helicase complex in the replisome, Cdc45 can be rate limiting for the initial DNA duplex unwinding during replication fork (re)start (Kohler, et al., 2016, Cell Cycle 15 974-985, which is herein incorporated by reference in its entirety). Reduction of Cdc45 increased conversion frequencies (see FIGS. 54 and 55). Apparently, interfering with replication fork restart increased time available for a sequence modification polynucleotide to anneal to a complementary DNA sequence near a stalled replication fork. Inhibition of Cdc45, by RNAi in this particular example, may synchronize or synergize with DLR as a block for a replication fork or replication fork restart and thus increase chances for an ssODN template (i.e., sequence modification polynucleotide) to anneal to its target site (see FIGS. 2, 3, and 5). Moreover, DLR mediated gene editing, as illustrated in FIG. 4, introduces a mismatch in a target (gene) where one stranded DNA could be considered “wild type” and the other as “mutant”. This mismatch may trigger a DNA repair process. There are at least three repair pathways that can address such a mismatch: two being Base Excision Repair and Base Excision Repair, which typically remove a mutation to conserve a parental sequence; another repair process being Mismatch Repair, which typically results in a mix of “wild-type” and “mutant” sequences in daughter cells. XRCC1 is a protein able to recognize specific DNA misfolded structures and it has been reported to be involved in Nucleotide Excision Repair and Base Excision Repair ((Hanssen-Bauer, et al., 2012, Int J Mol Sci 13 17210-17229, which is herein incorporated by reference in its entirety). These data support that these repair mechanisms competed Mismatch Repair. Whereas Mismatch Repair could result in gene conversion, Base/Nucleotide Excision Repair would likely preferentially restore a “wild type” sequence. Therefore, reduction of XRCC1, in this example, was favorable for usage of Mismatch Repair (i.e., in order to achieve a desired gene conversion), thus enhancing editing frequencies. Interestingly, a reduction of MSH2 resulted in a significantly lower conversion frequency (see FIG. 55). MSH2 is a critical component of Mismatch Repair (FIG. 4). Since incorporation of a complementary correction oligonucleotide generates a mismatch, these results suggested that Mismatch Repair was involved in this gene conversion process.

### Example 11: Modification of an Endogenous Genomic Target: BCL11A by DLR-Based RITDM Gene Editing

In this example, an enhancer in intron 2 of human BCL11A was targeted and edited by RITDM with a specifically-designed DLR molecule and a sequence modification polynucleotide. The present disclosure contemplates that, in some embodiments, disruption of this enhancer decreases expression of a transcriptional factor, BCL11A (Psatha et al., Mol. Ther. Methods Clin. Dev. 2018 Sep. 21; 10: 313-326, which is herein incorporated by reference in its entirety). In some embodiments, decreasing levels of BCL11A may increase fetal hemoglobin levels and/or decrease adult hemoglobin levels. (Bauer et al., Science, 2013 Oct. 11; 342(6155):253-257, which is herein incorporated by reference in its entirety). Without being bound by any particular theory, the present disclosure contemplates that increased production of fetal hemoglobin (HbF) and/or decreased production of adult hemoglobin (e.g., via gene editing of BCL11A) may ameliorate clinical symptoms of disorders involving adult beta-hemoglobin, such as B-thalassemia and sickle cell disease. Thus, this Example confirms that RITDM can be used to successfully genetically modify an endogenous disease-associated genotype within a mammalian genome by specifically converting a “GATAA” box into “GATTCC” in an enhancer in intron 2 of human BCL11A. Accordingly, this example demonstrates use of RITDM (e.g., a DLR-based genetic editing system) to modify disease-relevant nucleotide targets in mammalian cells by using a RITDM approach and system to genetically modify a human gene.

### Non-Sequence-Specific R-Element

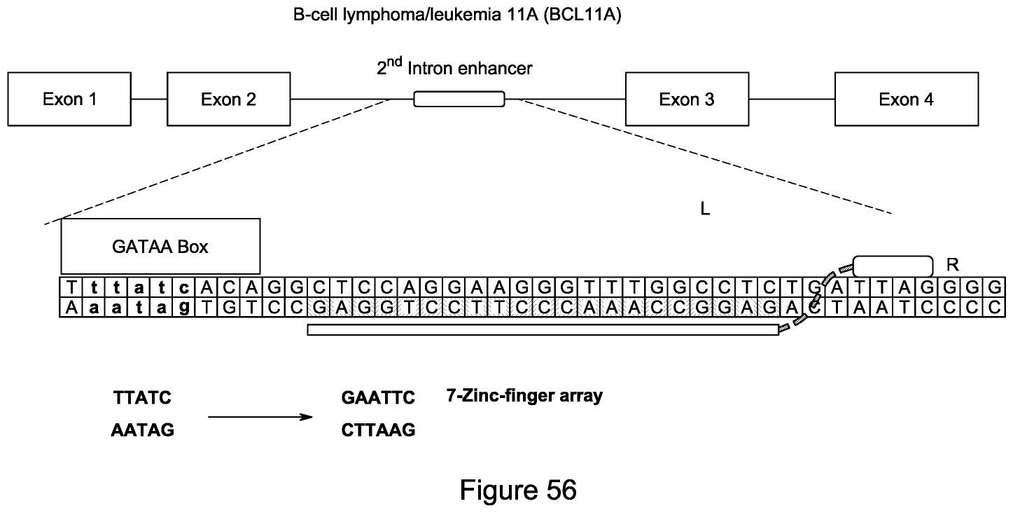

FIG. 56 is a schematic that depicts the approach used in this Example. This Example demonstrates editing in a “GATAA” box in an enhancer in intron 2 of human BCL11A in both HEK293 and U937 cells. Here, a DLR molecule (encoded on plasmid pb43 (full length DNA (SEQ ID NO. 159); cDNA (SEQ ID. NO.160); DLR amino acid sequence (SEQ ID. NO. 161)), which has a DNA recognition domain comprised in an array of 7 zinc-fingers, was designed to specifically recognize 5′-GAG-GCC-AAA-CCC-TTC-CTG-GAG-3′ (SEQ ID NO.162), a 21-nucleotide sequence on the lagging DNA strand (bottom row of nucleotides) of human BCL11A. FIG. 56 depicts a targeted “GATAA” box containing five nucleotides “GATAA” displayed as lowercase letters “gataa” in a 5′-to-3′ direction, 5′ upstream of this binding site; a complementary sequence, “TTATC”, is displayed as lowercase letters on the leading strand (top row of nucleotides) in FIG. 56. An R element was designed to bind to the strand opposite the “gataa” (here, the leading strand), in a non-sequence-specific manner. The sequence modification polynucleotide used was a 140-nucleotide single stranded DNA oligonucleotide containing the TTATC→GAATTC substitution roughly located in the middle of the length of this oligonucleotide. This sequence of the sequence modification polynucleotide used is provided as SEQ ID NO 163 (below) with an underlined and bold “GAATTC” to indicate the GAATTC sequence used in the TTATC→GAATC conversion.

(SEQ ID NO. 163)
5′CTCTTAGACATAACACACCAGGGTCAATACAACTTTGAAGCTA
GTCTAGTGCAAGCTAACAGTTGCTTGAATTCACAGGCTCCAGGAA
GGGTTTGGCCTCTGATTAGGGTGGGGGCGTGGGTGGGGTAGAAGA
GGACTGGC3′

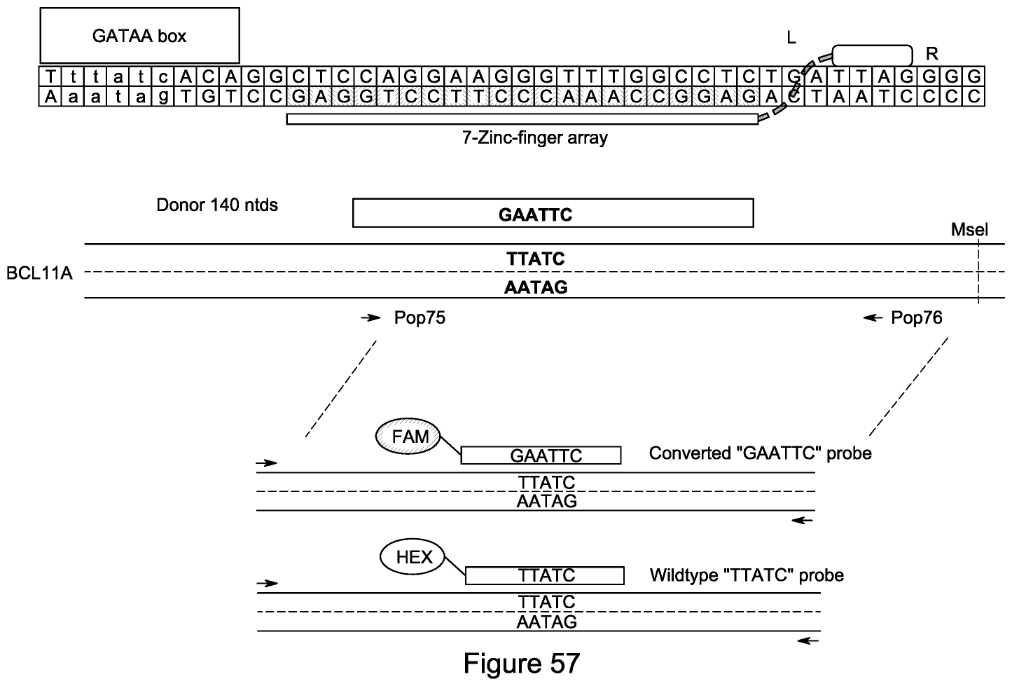

TTATC→GAATTC conversions after DLR-based gene editing were performed by droplet digital PCR (ddPCR). Relative positions of a sequence modification polynucleotide and position of a common primer pair (POP75, POP76, SEQ ID No.164, and 165) are also depicted in FIG. 57. As also depicted in FIG. 57, one common primer, POP75, is located within this sequence modification polynucleotide sequence, while POP76, is located outside of this sequence modification polynucleotide sequence. Allele-specific probes conjugated with fluorophores FAM and HEX were designed to distinguish between “GAATTC” and “TTATC” respectively. MseI restriction enzyme sites (location) indicated in FIG. 57 with a vertical, dashed line) were used in preparations for ddPCR reactions.

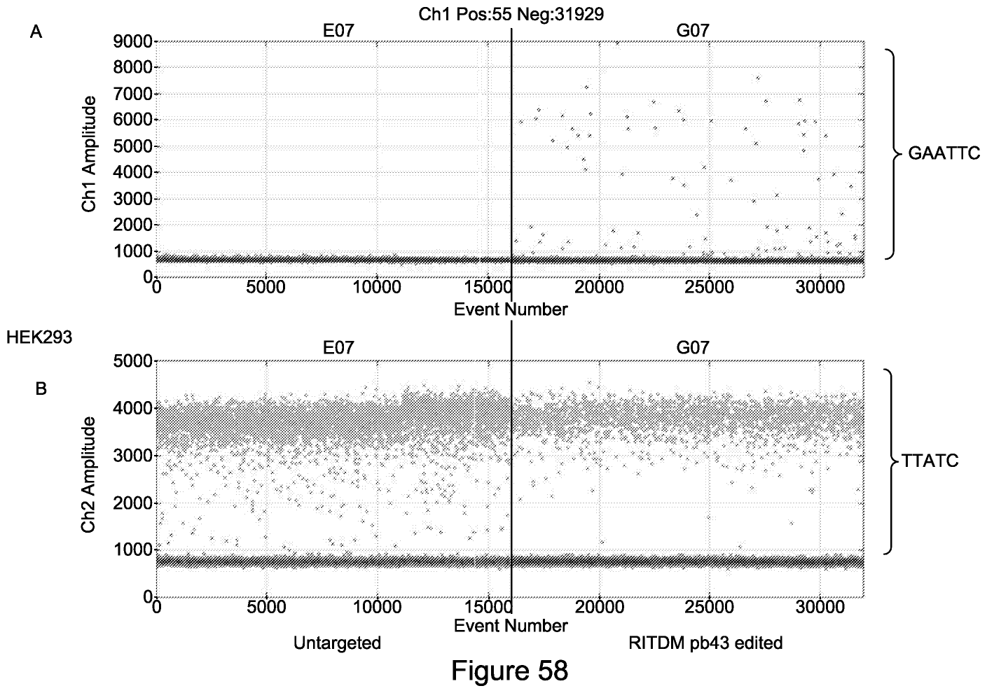

FIG. 58 confirms successful TTATC→GAATTC genetic conversion at an enhancer in intron 2 of human BCL11A as measured by ddPCR and depicted on dot (droplet) plots. After transfection of HEK293 cells with plasmid pb43 and the 140-nucleotide sequence modification polynucleotide, cells were allowed to recover and grow on complete culture medium, containing 15% FBS in DMEM, for five days. After five days, genomic DNA was isolated and used in ddPCR analysis. The raw droplet data depicted in FIG. 58 represent “GAATTC” droplets in FIG. 58A (top panel) and “TTATC” droplets in FIG. 58B (lower panel). Both panels 58A and 58B are divided with a line that separates negative control cells (untransfected)) from those cells transfected with pb43 and the 140-nucleotide sequence modification polynucleotide. The data show that only “TTATC” droplets were detected in the negative control condition whereas “GAATTC” droplets were detected in HEK 293 cells transfected with pb43 and the 140-nucleotide sequence modification polynucleotide. These data confirm successful targeting and editing using a DLR molecule in combination with a sequence modification polynucleotide to achieve a targeted conversion of TTATC→GAATTC in enhancer in intron 2 of BCL11A.

Detailed genomic TTATC→GAATTC conversion validation and background damage evaluation as measured by next generation sequencing after DLR-based gene editing was also performed. Next generation sequencing of targeted HEK293 pooled cells (and untransfected HEK293 as control) was done. Genomic DNA was isolated and used as a template on which a 197-bp PCR amplicon surrounding a “GATAA” box in an enhancer of intron 2 of BCL11A was generated by using a primer set of POP75 and POP76. Amplified PCR products from edited HEK293 cells and control HEK293 cells were analyzed for indels and SNPs on an Illumina next generation sequencing platform (GENEWIZ, South Plainfield, NJ). In particular, SNP analysis was performed to confirm TT→GA conversion and indel analysis to confirm a one-nucleotide insertion between nucleotide “A” and “T” within the GATTA box.

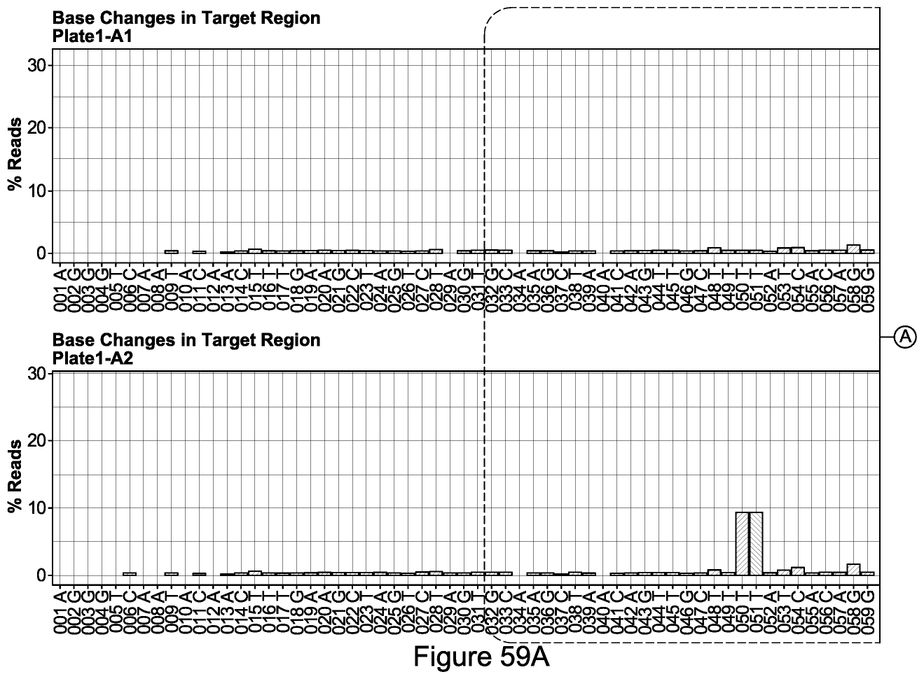

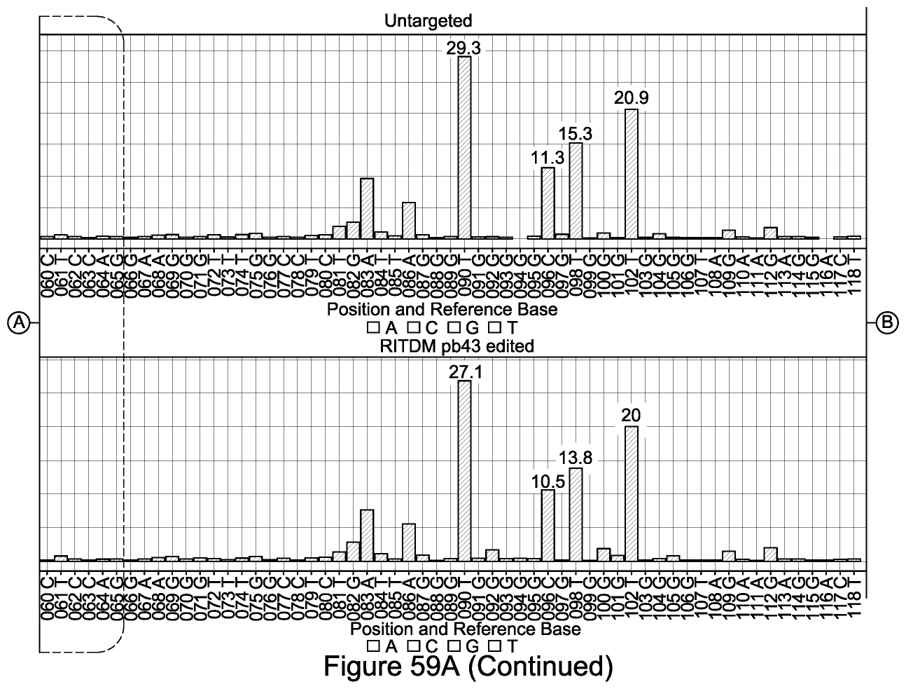

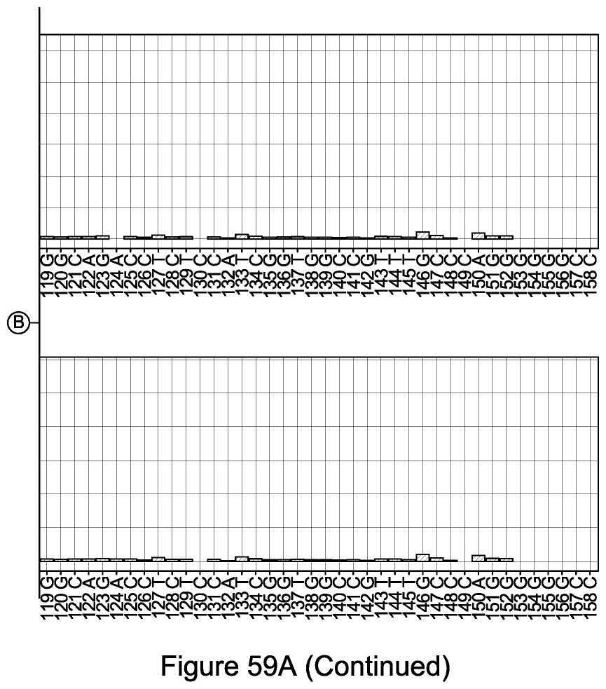

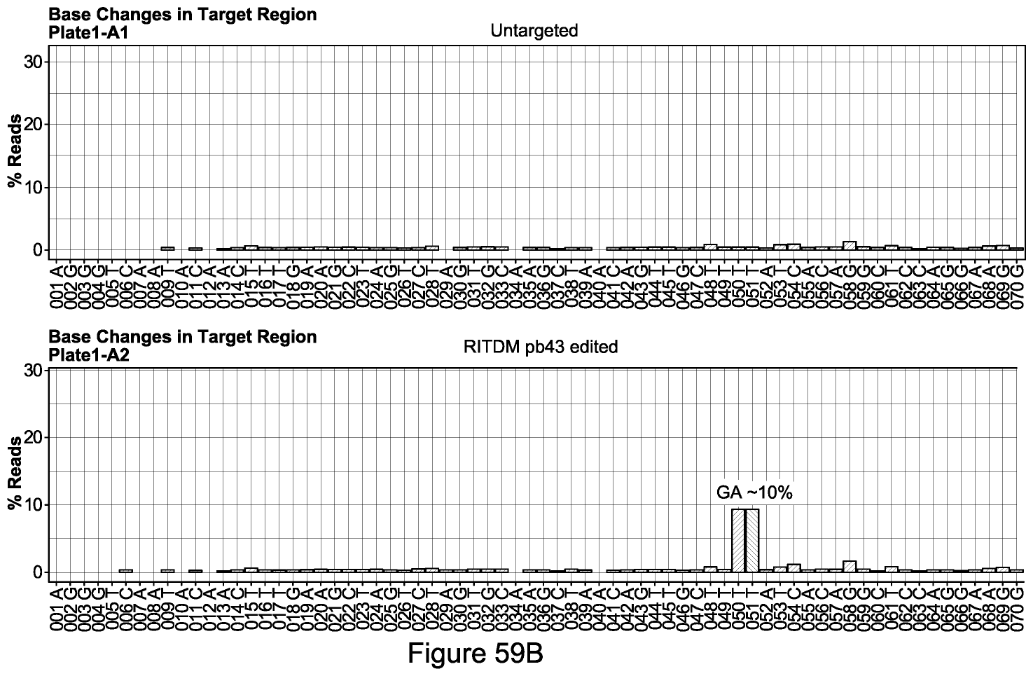

FIGS. 59A and 59B depict results that confirm detection of single nucleotide TTATC→GAATTC conversion at this target site. In addition, single nucleotide polymorphisms (SNPs) analysis within a target region surrounding a “GATTA” box of this BCL11A locus was performed. FIG. 59A shows overall views of SNPs analysis at these target sites obtained with untargeted HEK293 cells, and RITDM targeted pooled HEK293 cells. Bar graphs plot frequencies of SNPs at each nucleotide position in this 197 bp PCR amplification region. FIG. 59B is a magnified view of a portion close to this gene editing site. In this example cells transfected with pb43 and a correction template showed a desired TT-to-GA conversion at these expected nucleotide positions with a frequency of approximately 10%. That is, compared to non-transfected HEK293 cells, no other nucleotide conversions were detected at a level 10% above background levels. In addition to the targeted genetic conversion using the sequence modification polynucleotide, a number of additional SNPs were detected; importantly, since these SNPs were detected in both targeted and untargeted (i.e., control/untransfected) samples, it seems most likely that sequences within this 197 bp amplicon are different from reference sequences reported in reference databases, for example, a RefSeq for a wild-type gene sequence as shown in SEQ ID NO:193. That is, both targeted and untargeted samples show almost identical patterns and frequencies of SNPs in this particular region, thus, effects other than the targeted TTATC→GAATTC cannot be attributed to RITDM editing. In summary, genomic editing at significant frequencies was achieved by RITDM and as compared to untransfected cells, no “off target” nucleotide changes were detected.

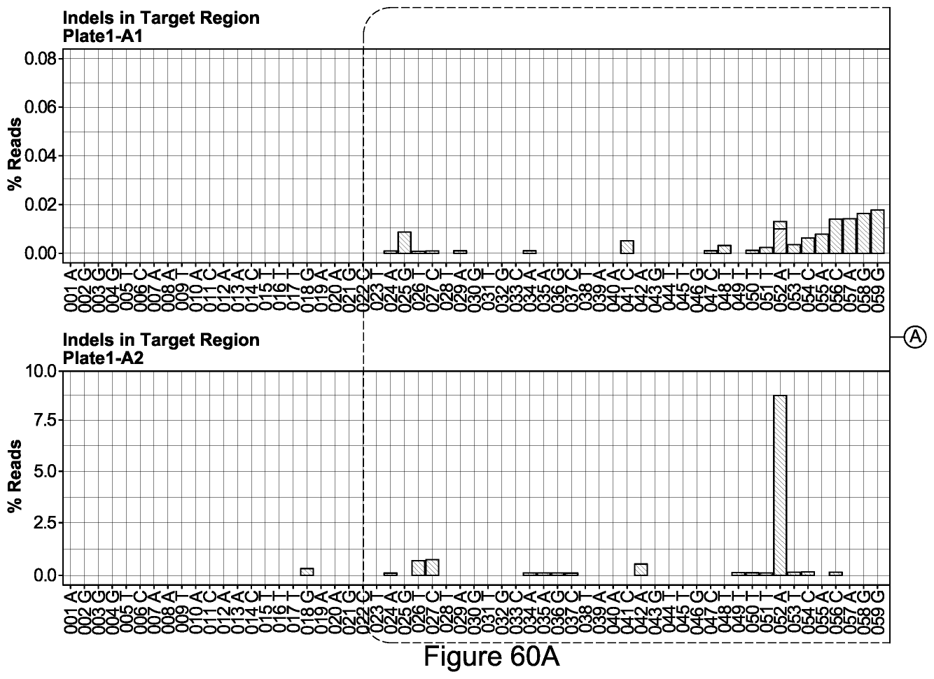

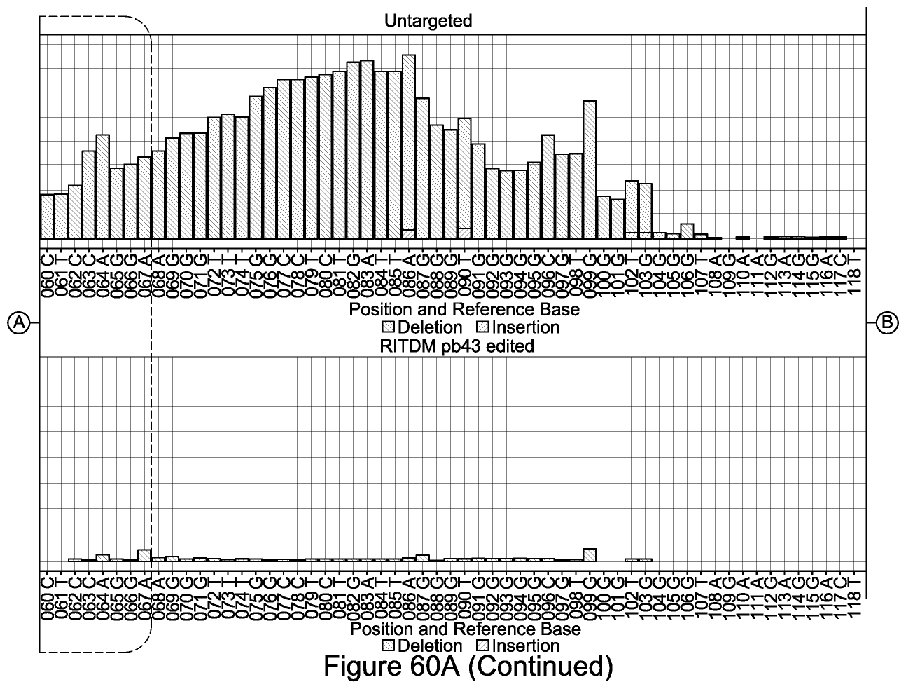

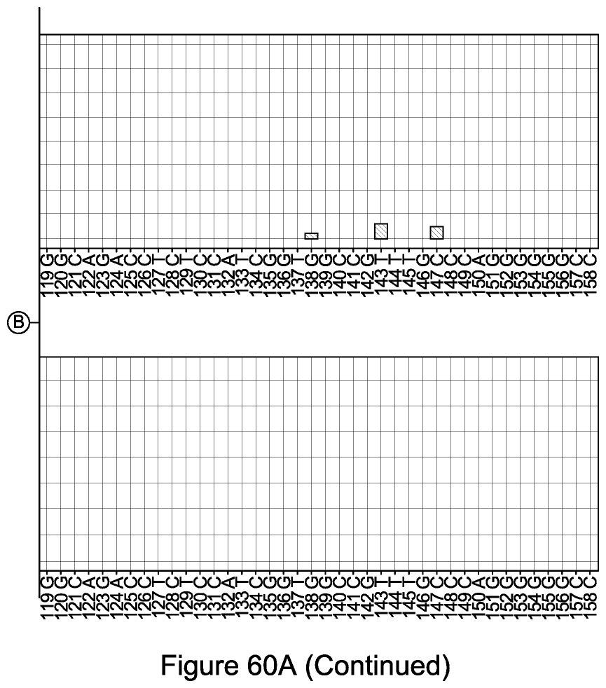

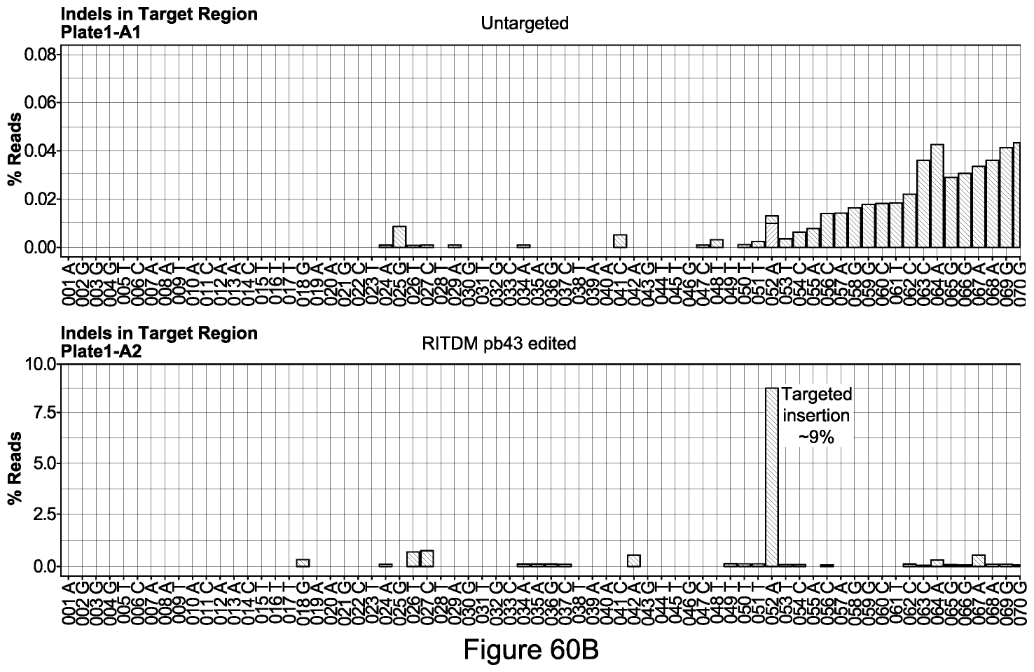

FIGS. 60A and 60B show insertion and deletion analysis around a “GATAA” box in an enhancer in intron 2 of BCL11A as depicted by a frequency plot of insertions and deletions analysis for untargeted (i.e., untransfected) HEK293 cells and targeted pooled HEK293 cells.

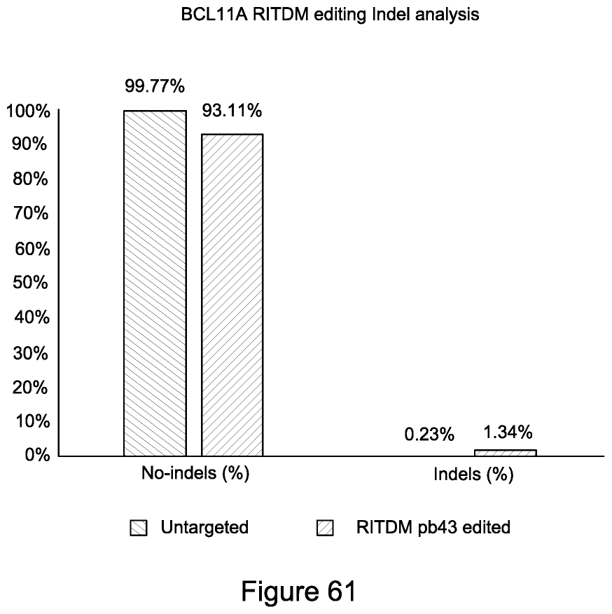

FIG. 60A shows overall views of indels analysis at these target sites obtained from these two cellular populations. Bar graphs plot frequencies of insertions and deletions at each nucleotide position of this 197 bp PCR amplification region. Compared to untargeted cells, a single nucleotide insertion was detected at the target site in edited cells with a frequency of approximately 9%. FIG. 60B is a magnified view of a portion close to the targeted site in the BCL11A gene. In combination with SNP analysis, a genomic conversion of TTATC→GAATTC was confirmed at a frequency of approximately 9-10% in HEK 293 cells after being targeted and edited by pb43 in combination with the 140-nucleotide sequence modification polynucleotide as described herein. FIGS. 60A and 60B also confirm an overall very low frequency of insertions and/or deletions. As shown in FIG. 61, overall indel frequencies were 0.25% in untargeted cells and 1.34% in targeted cells; no larger indels were detected in targeted cells.

This Example also confirms important safety features of this approach to gene editing. As a very low level of insertions and deletions was detected, technologies described and exemplified herein enable targeted gene conversion without potentially detrimental generation of insertions, deletions and/or undesired single nucleotide polymorphisms at significant levels as may be observed in other types of gene editing technologies. Also important is that the data provided herein further confirm the safety, efficiency, and efficacy of technologies of the present disclosure. That is, modification agents (e.g., polymeric modification agents, e.g., DLR molecules) successfully edited nucleic acid sequences and also triggered repair pathways that did not cause significant levels of undesired or unexpected sequence modifications or rearrangements (e.g., chromosomal changes or tandem integration of correction templates). That is, technologies of the present disclosure successfully and efficiently achieve gene editing without relying on nuclease or nickase activity and/or without appearance or creation of significant levels of undesired and/or unexpected DNA changes (i.e., no significant or low levels of “off-target” effects), while achieving relatively high editing frequencies.

The results of this example confirm and extend that RITDM systems and approaches provide both a strong safety profile and impressive gene editing efficiency.

### Sequence-Specific R-Element

In addition to a non-sequence specific R element, data also confirm and support that a sequence-specific R element can achieve targeted gene editing.

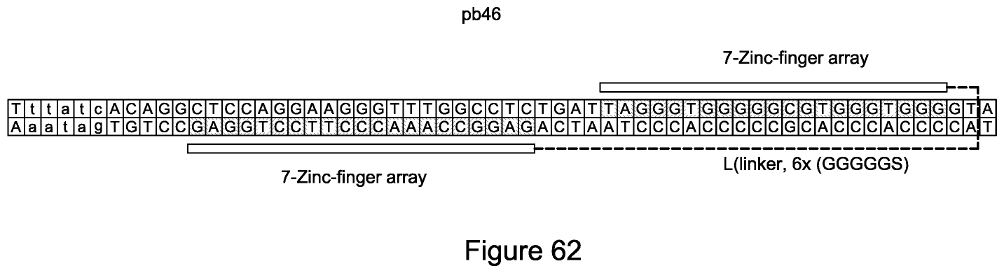

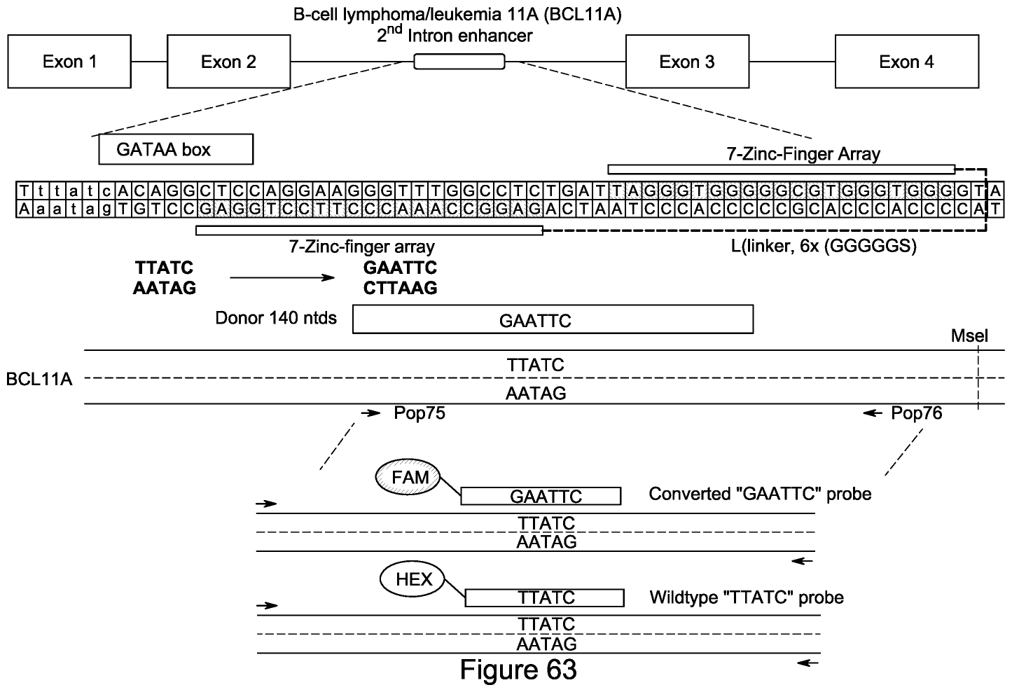

Specifically, FIG. 62 provides a schematic depicting a DLR molecule, encoded on plasmid pb 46 (full length DNA (SEQ ID NO. 166) cDNA (SEQ ID. NO.167), DLR amino acid sequence (SEQ ID. NO. 168)), that comprises two 7-zinc-finger arrays recognizing 5′-GAG-GCC-AAA-CCC-TTC-CTG-GAG-3′ (SEQ ID NO.162), a 21-nucleotide sequence on the lagging strand of human BCL11A as a D-element and 5′-TAG-GGT-GGG-GGC-GTG-GGT-GGG (SEQ ID NO.169), a 21-nucleotide sequence on the leading strand of this target sequence as an R-element. These two zinc-finger arrays were connected with a linker. A similar editing approach, as well as ddPCR detection strategy were used as described herein (i.e., in the non-sequence specific R-element portion of this Example) and are illustrated in FIG. 63. U937 cells were used in this example.

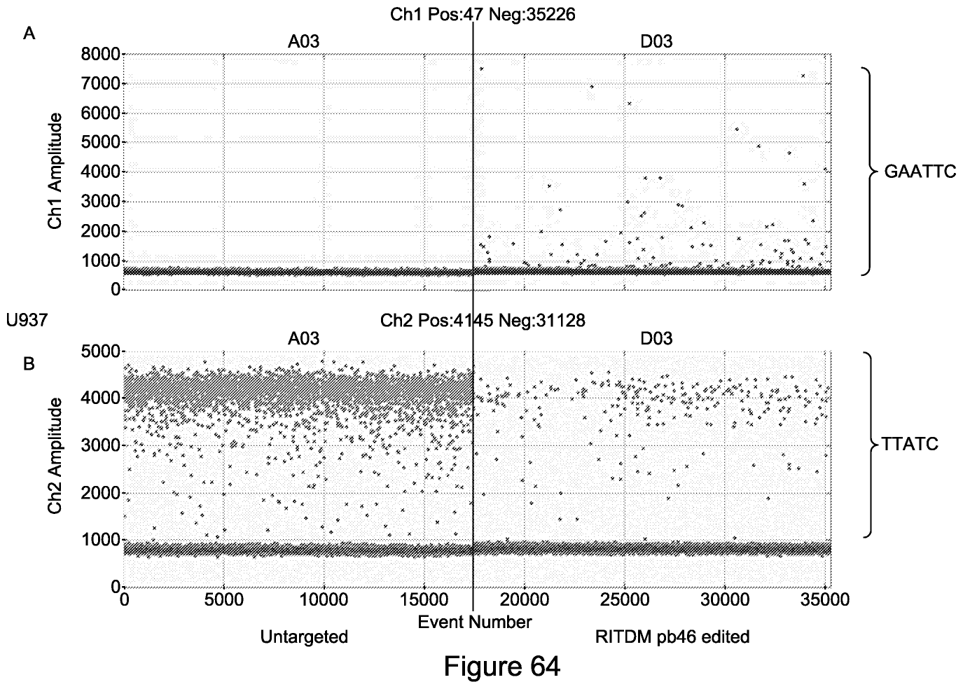

FIGS. 64A and 64B demonstrate that, as confirmed by ddPCR, a “GATAA” box in an enhancer in intron 2 of human BCL11A gene were successfully targeted and edited by DLR molecules with double zinc-finger arrays. In the upper panel, untargeted U937 cells shows no positive droplet population corresponding to “GAATTC.” After cells were transfected with pb46 and a donor template, a targeted cell population containing “GAATTC” was identified using ddPCR detection (with a fam conjugated probe) as shown in FIG. 64A (upper panel). “TTATC” droplets, indicating untargeted cells, are shown in the FIG. 64B (lower panel). These data confirm that a DLR molecule with dual zinc-finger arrays in combination with a sequence modification polynucleotide can be used for successful TTATC→GAATTC genetic conversion at a “GATAA” box in an enhancer of intron 2 of human BCL11A. Importantly, as discussed herein, these data also confirm that modification agents of the present disclosure (e.g., comprising zinc-finger arrays) do not appear to display any cleavage activity and, thus, as provided herein, nucleic acid modifications are effectively, efficiently, and safely made in the absence of any cleavage-based method.

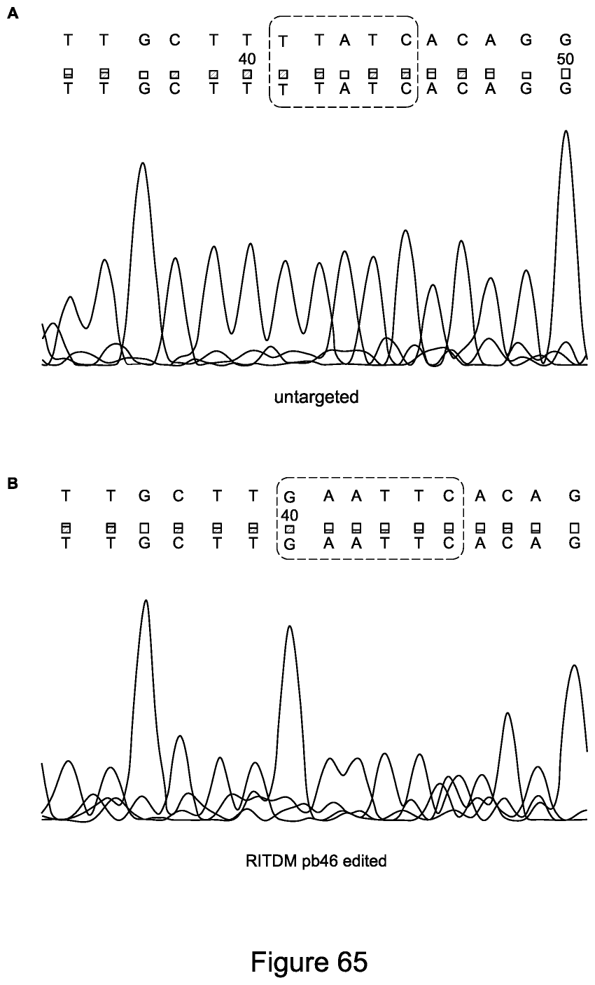

FIGS. 65A and 65B show Sanger sequencing results used to confirm successful targeting and repair at a “GATAA” box in an enhancer of intron 2 of human BCL11A. FIG. 65A demonstrates an exemplary chromatogram of a “GATAA” box in an enhancer from untargeted U937 cells by Sanger Sequencing. FIG. 65B shows a converted “GAATTC” sequence after RITDM targeting with pb46 and donor template.

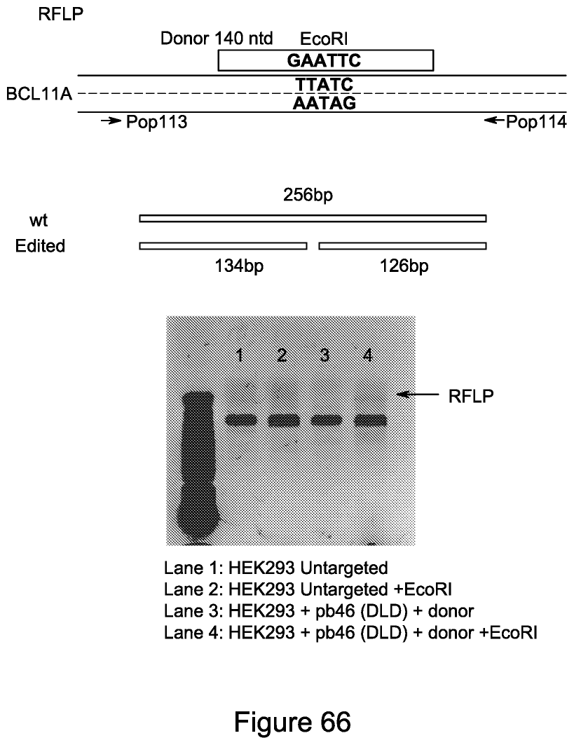

These results confirm that a DLR molecule and sequence modification polynucleotide can be used to successfully, efficiently, and effectively target endogenous gene conversion in mammalian cells without a need for, e.g., DNA breakage or cleavage by an exogenous agent. The TTATC→GAATTC conversion at a “GATAA” box in an enhancer in intron 2 of human BCL11A gene, as described herein, creates an EcoRI restriction enzyme recognition site at this target locus. Accordingly, PCR amplicons that contain this “GAATTC” genetic conversion can be cut by digesting with an EcoRI restriction enzyme. In FIG. 66, a restriction fragment of length polymorphism (RFLP) is shown to further confirm successful targeting and editing via RITDM using a DLR molecule (pb46) and sequence modification polynucleotide. Two end primers, POP113 (SEQ ID NO.170) and POP 114 (SEQ ID NO.171) were designed to amplify a target region flanking this donor template, which contains a “GAATTCC” sequence approximately in the middle of the length of the sequence. PCR amplification was performed using POP 113 and POP 114 yielding 256 bp DNA products. PCR reactions using these two primers were designed to amplify both unedited and edited sequences in pools of U937 cells targeted by RITDM; however, only amplicons with a “GAATTC” conversion can be digested by an EcoRI restriction enzyme to yield two fragments, one of 134 bp and another of 126 bp in size. Since these two fragments are of similar length, it is difficult to resolve using gel electrophoresis, but they can be observed as a single band and are visibly smaller than the undigested PCR amplicon. Observation of this smaller band on an agarose gel can also be used to confirm successful genetic TTATC→GAATTC conversion. FIG. 66, shows RFLP results after electrophoresis on a 2% agarose gel confirming successful RFLP detection of an EcoRI digested DNA band. PCR amplicons were electrophoresed side-by-side with and without EcoRI restriction enzyme digestion. Untargeted U937 cells did not result in detection of RFLP products after EcoRI digestion (shown in lane 2), while in targeted cells EcoRI digestion clearly showed a smaller band (arrowed) in lane 4. These data further confirm that a RITDM system of the present disclosure is able successfully, efficiently, and effectively achieve precise gene editing.

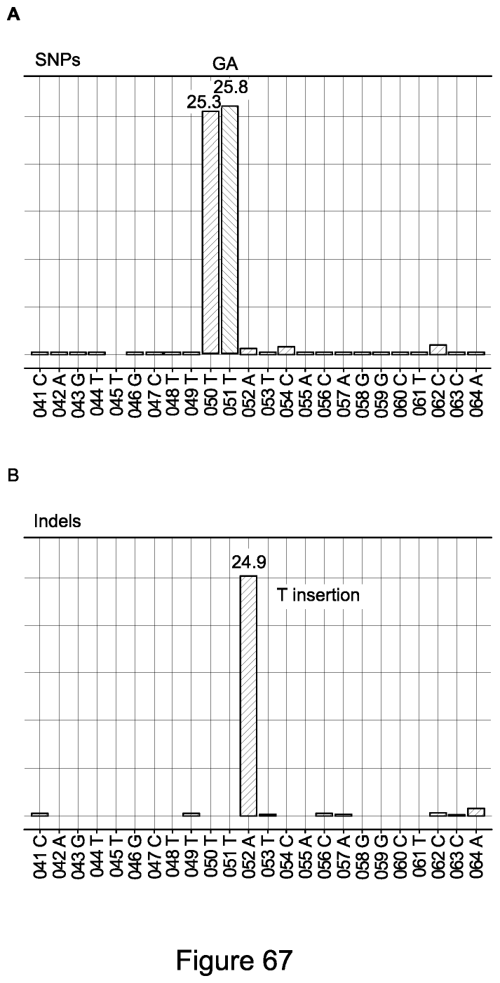

FIG. 67 shows data confirming successful genetic TTATC→GAATTC conversion with a frequency of approximately 25%, after using pb46, and sequence modification polynucleotide as described herein. Since this conversion involves both a nucleotide insertion and a nucleotide change, it is represented in both SNP analysis and indel analysis as measured by next generation sequencing. FIG. 67A shows frequencies of a TT→GA conversion (25.8%) by SNP analysis. FIG. 67B shows frequencies of a T insertion at a desired position by Indel analysis (24.9%). Collectively, these results further confirm that RITDM systems and technologies of the present disclosure can be used to precisely target and edit genetic sequences.

### Example 12: Modification of an Endogenous Genomic Target: Exon 51 of Dystrophin Gene by DLR-Based RITDM Gene Editing

In this example, exon 51 of the human dystrophin gene, DMD, was targeted and edited using a RITDM approach to change the dystrophin reading frame via two-nucleotide of insertion by RITDM, using specifically designed DLR molecules and a single stranded oligonucleotide template (i.e., a sequence modification polynucleotide). Duchenne muscular dystrophy (DMD) is an X-linked disease caused by mutations in the dystrophin and presents, clinically, throughout the entire body, a progressive muscle wasting disease. One commonly occurring DMD-causing mutation is a deletion of exon 50 of the human dystrophin, which causes a frame shift and distorts dystrophin translation such that little to no functional dystrophin protein is produced. One known manner in which any detrimental impact of such mutations (e.g., deletion of exon 50) can be overcome is by skipping exon 51 using antisense oligonucleotides to “mask” exon 51, thereby restoring the dystrophin reading frame and resulting in functional (albeit shorter) dystrophin protein which results in a milder clinical phenotype as compared to DMD; however as masking techniques do not change the underlying genetic code, they still requires continuous treatment to mask genetic mutations in order to make dystrophin (Falzarano et al., Molecules. 2015 October; 20(10):18168-18184, which is herein incorporated by reference in its entirety). As described in the present Example, a RITDM system with a specifically-designed DLR molecule and sequence modification polynucleotide can successfully edit the dystrophin gene by inserting two nucleotides into exon 51 such that a normal reading frame is achieved.

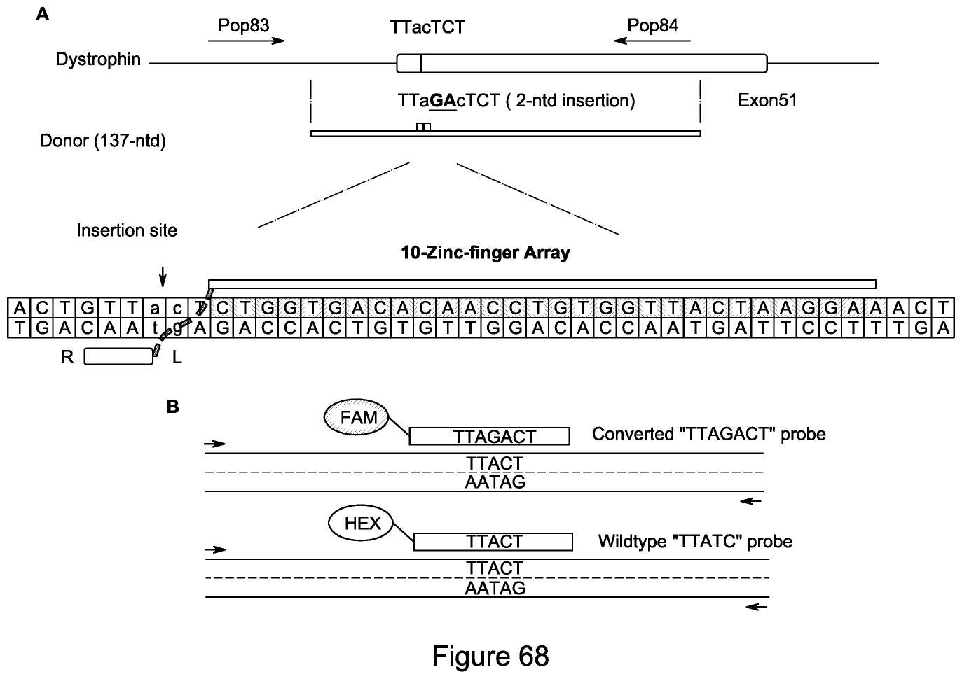

FIG. 68A is a schematic illustrating the editing strategy used in this Example. U937 cells were used and a DLR molecule, encoded on plasmid pb49 (full length DNA (SEQ ID NO. 172); cDNA (SEQ ID. NO.173); DLR amino acid sequence (SEQ ID. NO. 174)), has a DNA recognition domain which was an array of 10 zinc-fingers, specifically designed to recognize 5′-CTG-GTG-ACA-CAA-CCT-GTG-GTT-ACT-AAG-GAA-3′ (SEQ ID NO.175), a 30-nucleotide sequence on the leading strand of human dystrophin. An R element was designed to bind to an opposite strand, in this case the lagging strand, in a non-sequence-specific manner. A 137-nucleotide single stranded DNA oligonucleotide with a desired TTACTCT→TTAGACTCT (SEQ ID NO. 245) substitution roughly located in the middle of the length of this oligonucleotide served as the sequence modification polynucleotide. A two-nucleotide sequence “GA” was inserted between “a” and “c” of sequence “TTacTCT” in exon 51 of a dystrophin gene and resulted in an altered reading frame in exons downstream of the insertion The sequence of the sequence modification polynucleotide used in this Example is provided below with the “GA” insertion indicated in underline and bold.

(SEQ ID NO. 176)
5′TAATTTTTCTTTTTCTTCTTTTTTCCTTTTTGCAAAAACCCAA
AATATTTTAGCTCCTACTCAGACTGTTAGACTCTGGTGACACAAC
CTGTGGTTACTAAGGAAACTGCCATCTCCAAACTAGAAATGCCAT
CTTCC 3′

Detection of a genetic “GA” insertion after DLR-based gene editing was performed by droplet digital PCR (ddPCR). Relative positions of the sequence modification polynucleotide and position of a common primer pair (POP83, POP84, SEQ ID No.177, and 178) are also indicated in FIG. 68B. One common primer, POP83 was located outside the sequence modification polynucleotide sequence, while POP84, located inside. Allele-specific probes conjugated with fluorophores FAM and HEX were designed to distinguish between “GA” and wild-type respectively.

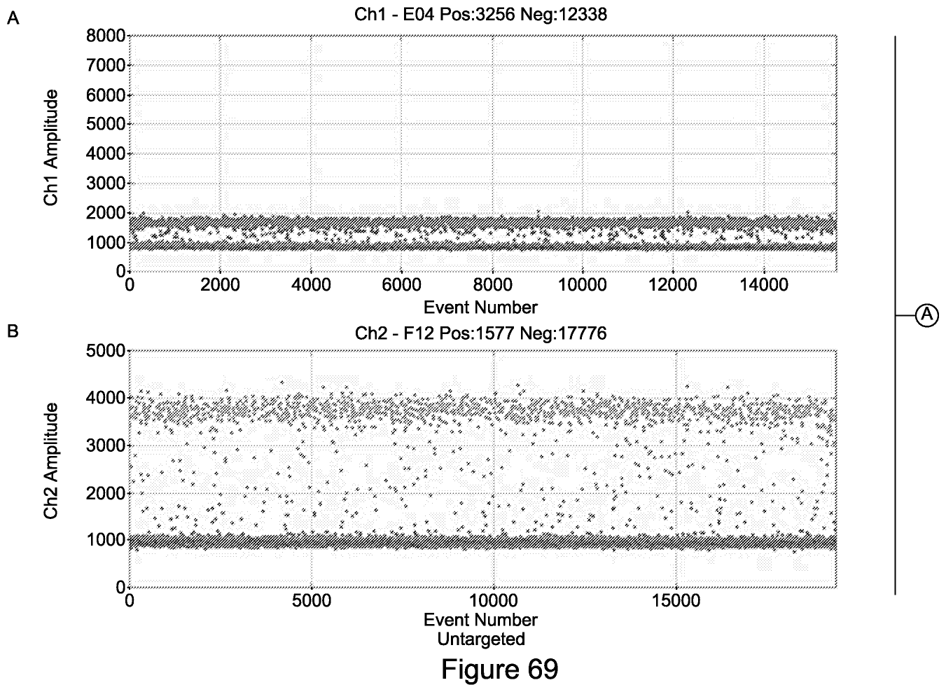

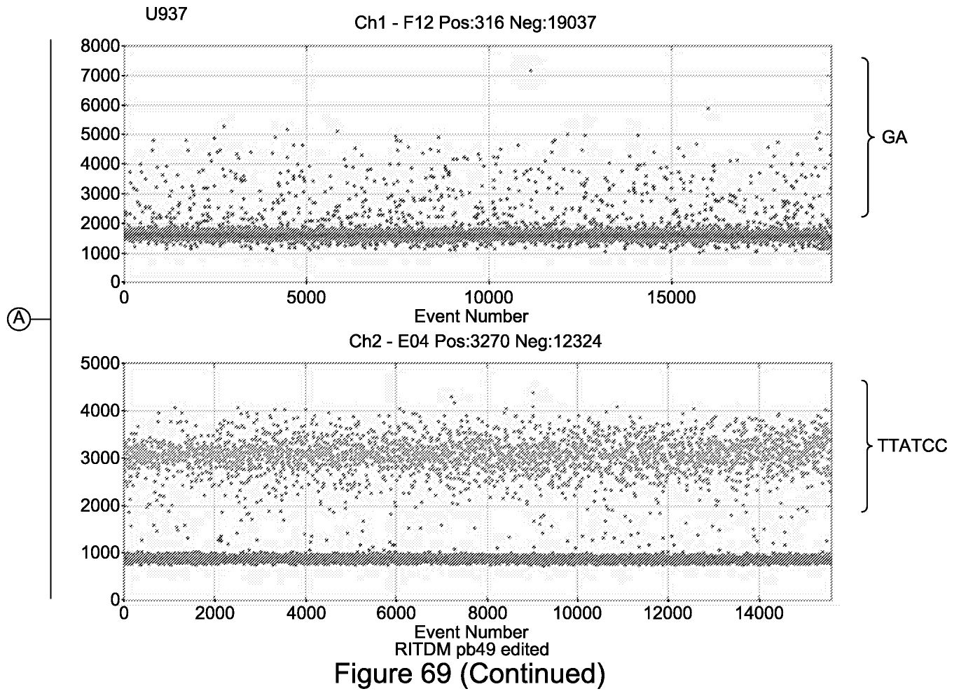

FIG. 69 illustrates successful “GA” insertion in exon 51 of dystrophin in U937 cells as measured by ddPCR. In this example, after transfection of U937 cells with a DLR molecule and sequence modification polynucleotide (plasmid pb49 and the 137-nucleotide correction template, respectively), cells were allowed to recover and grow on complete culture medium, containing 15% FBS in DMEM, for five days. After five days genomic DNA was isolated and used in ddPCR analysis. Raw droplet data are shown FIGS. 69A and 69B. That is, successful edited is confirmed by detection of “GA” insertion droplets as shown in FIG. 69A (top panel) and “wild-type” (those without “GA” insertions) droplets are displayed in FIG. 69B (the lower panel). Untargeted cU937 cells were used as negative control and resulted in only wild-type droplets. After U937 cells were transfected with pb49 and sequence modification polynucleotide containing the “GA” insertion, ddPCR demonstrated successful targeted integration of “GA” into exon 51 of the human dystrophin gene.

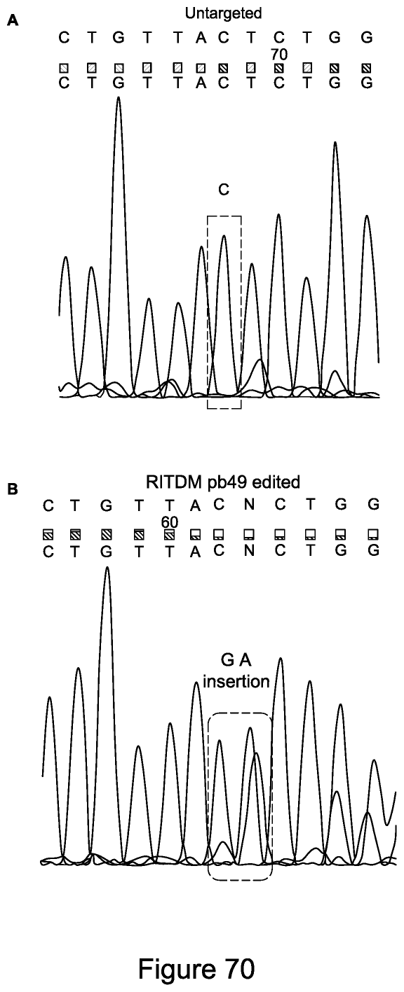

FIGS. 70A and 70B show Sanger sequencing results used to further confirm successful targeting and editing of exon 51 of the human dystrophin gene. FIG. 70A shows an exemplary chromatogram of a wild-type “TTACT” sequence from untargeted U937 cells by Sanger sequencing. FIG. 70B shows an edited “TTACT” sequence at this target site after RITDM editing with pb49 and the sequence modification polynucleotide containing the two-nucleotide “GA” insertion relative to wild-type. Sequencing results confirm detection of this two-nucleotide “GA” insertion into the targeted location and, after this insertion, two reading frames are present. These results confirm that a DLR molecule in combination with a sequence modification polynucleotide can successfully target and edit a sequence in an endogenous mammalian gene in mammalian cells to successfully modify a disease-causing genotype.

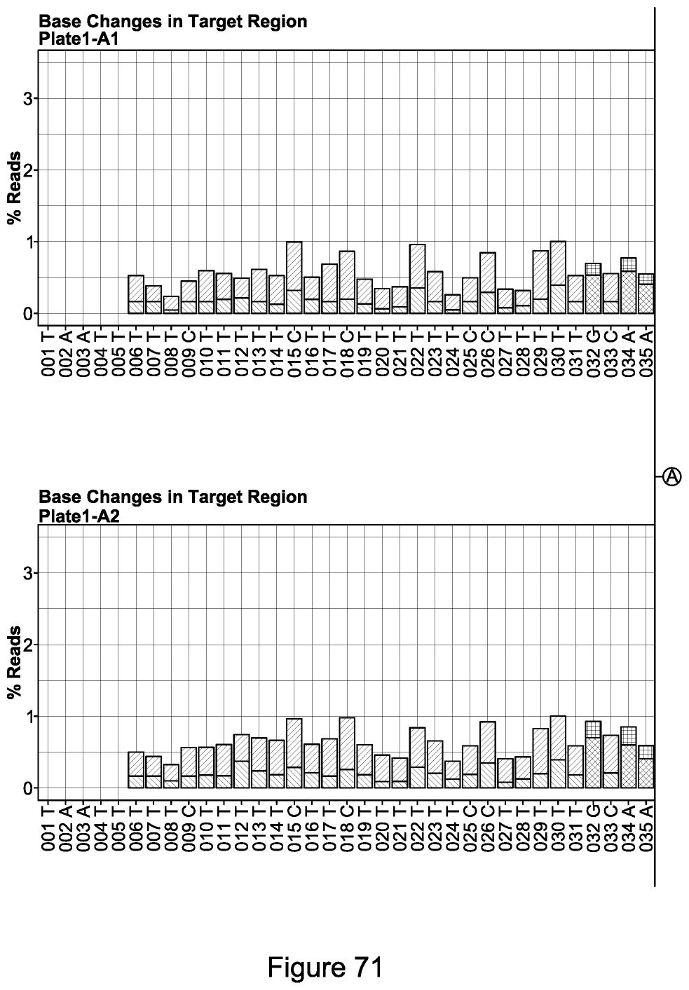

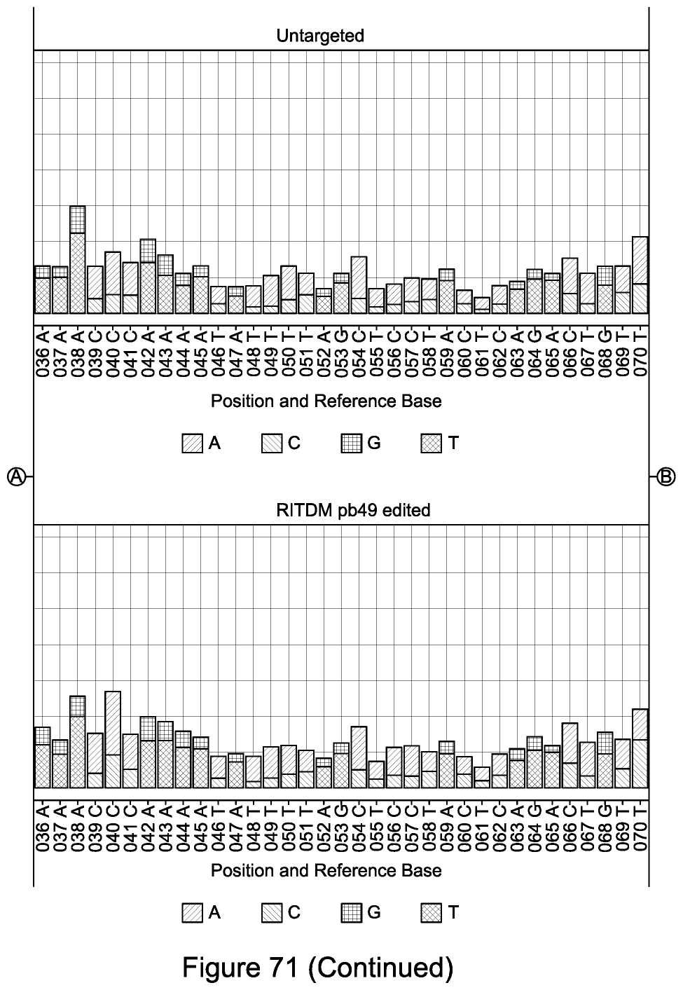

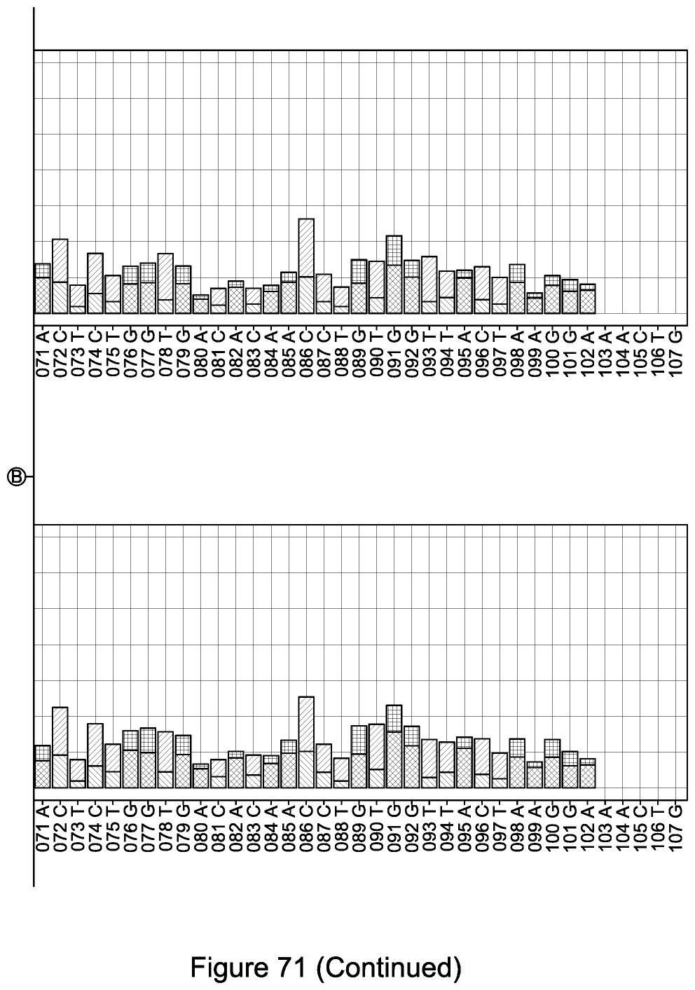

Further detailed validation of this genomic “GA” two-nucleotide insertion and evaluation of whether any background changes (e.g., off-target changes, e.g., potentially detrimental off-target changes) occurred were performed by next generation sequencing. Next generation sequencing of targeted U937 pooled cells was performed; untransfected U937 cells served as a control condition. Genomic DNA was isolated and used as a template on which a 151-bp PCR amplicon was generated by using a primer set of POP83 and POP84 (in which is also the primer set used in ddPCR analysis in this Example). Amplified PCR products from targeted U937 cells and control untransfected (and thus, untargeted) U937 cells were analyzed for indels and SNPs on an Illumina next generation sequencing platform (GENEWIZ, South Plainfield, NJ). FIG. 71 shows a SNPs analysis comparing untargeted and targeted U937 cells. A SNP spectrum at each position within this amplification region, shows that these two cellular population were almost identical with no significant nucleotide frequency differences. Average SNP frequencies at each position in both population were below 2% of total reads. These data again demonstrate that targeting by RITDM did not create significant levels of mutations. SNPs detected were comparable between these populations and most likely due to background noise in genetic analysis methods.

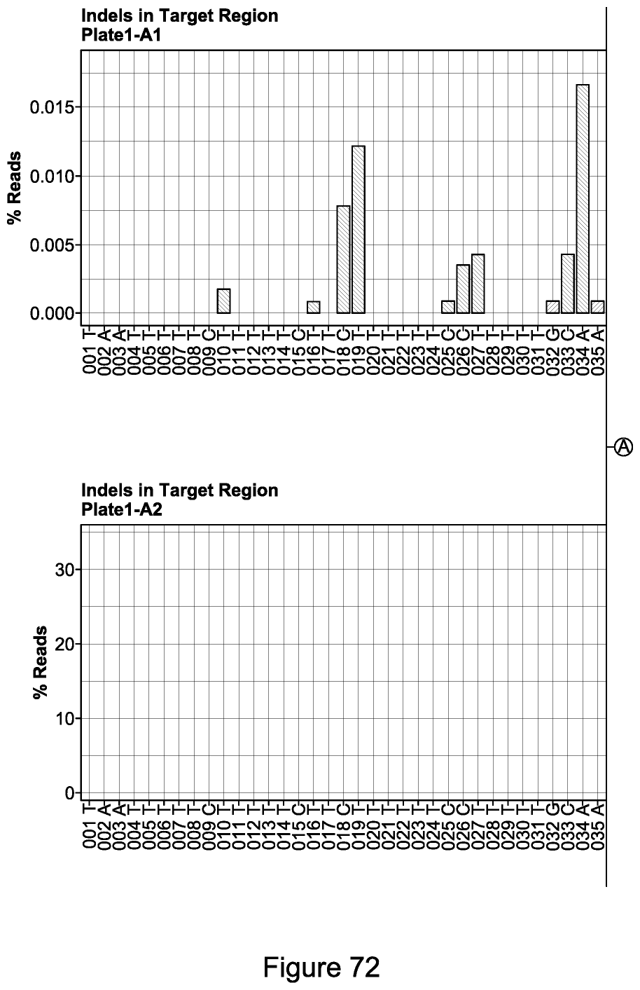

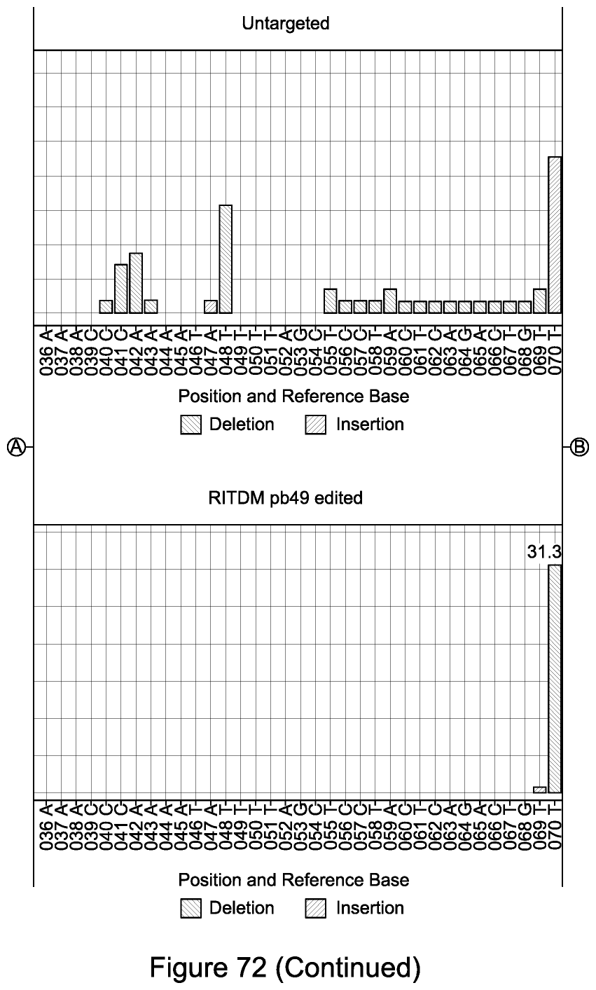

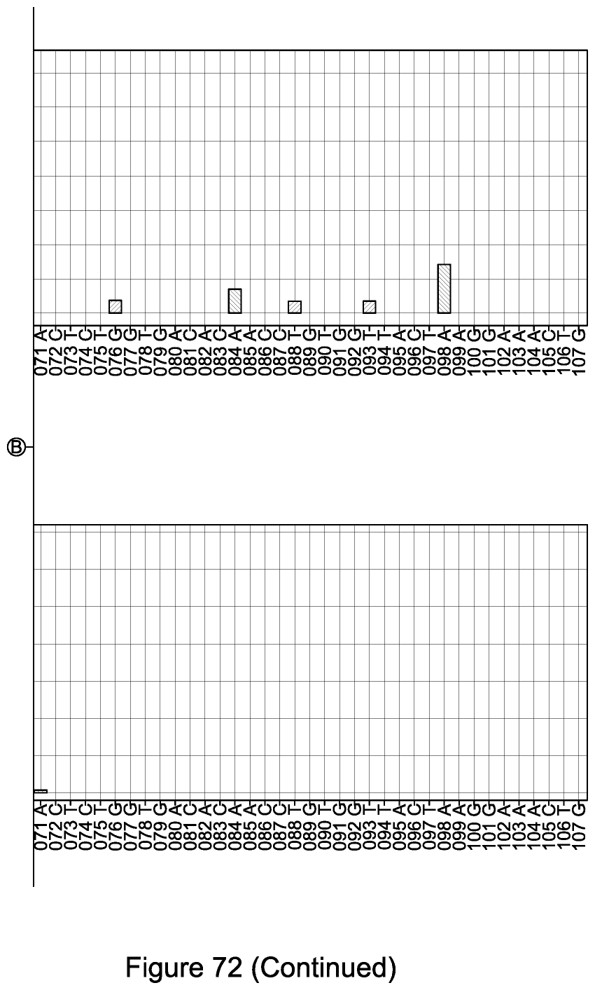

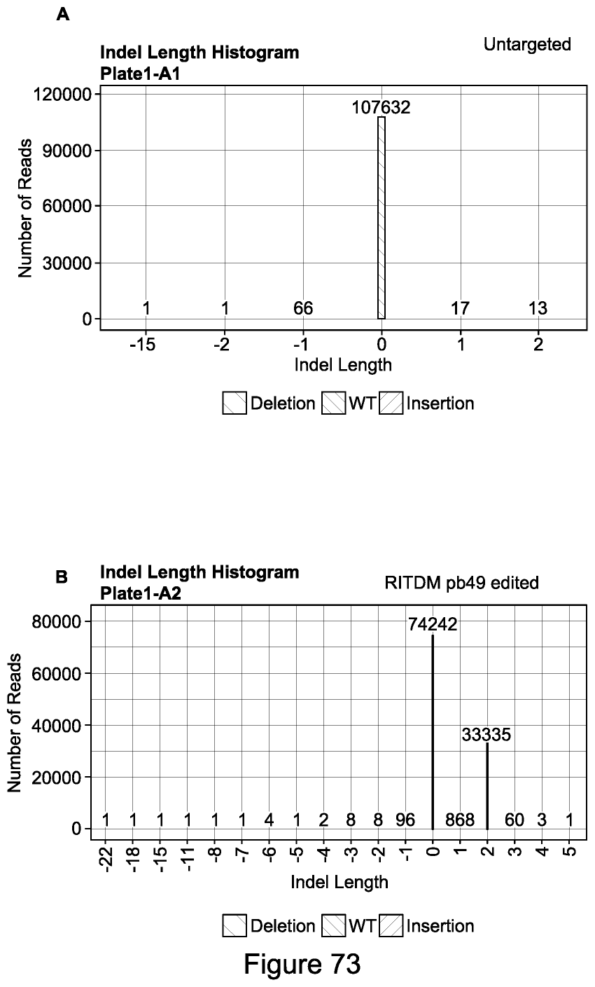

FIG. 72 shows an indel analysis between untargeted and targeted U937 pooled cell populations. Bar graphs plot frequencies of insertions and deletions at each nucleotide position of this targeted amplification region of exon 51 of the human DMD gene. The upper panel shows an indel analysis at each position from untargeted U937 cells as background reference. The lower panel shows an indel analysis from targeted U937 cells. As can be seen in this figure, we calculated a frequency of 31.3% of insertions at this desired position of a “TTACT” targeting site. When looking at this figure however, this indel analysis does not distinguish how many nucleotides are inserted at a specific position. Next, an indel length histogram in FIG. 73 elaborated on length changes of entire sequence reads. FIG. 73A shows an indel length histogram from untargeted U937 pooled cells: only 13 reads comprised two-nucleotide insertions among 107632 “wild-type” reads. FIG. 73B shows a histogram with 33,335 reads that had a two-nucleotide insertion, which is approximately 30% of reads compared to wild-type reads. This frequency is similar to that of an indel analysis as shown in FIG. 71. Collectively, next generation sequencing confirmed and validated successful insertion of a frame-shifting two-nucleotide sequence, and demonstrates that technologies of the present disclosure are capable of changing a reading frame (e.g., of exon 51 of human dystrophin).

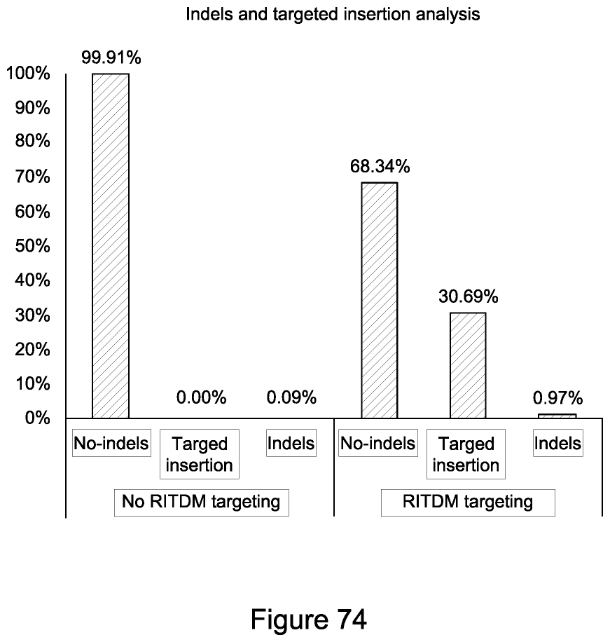

FIG. 74 shows overall indels and editing frequencies of a targeted U937 pooled cellular population comparing to an untargeted control. After RITDM targeting with pb49 and a sequence modification polynucleotide, an overall RITDM editing frequency of 30.69% and an indel frequency of only 0.97% was observed. In this untargeted population, an indel frequency of 0.09% was observed. Taken together, RITDM mediated gene editing is able to achieve relatively high gene editing efficiency with very low indel frequencies.

### Example 13: Genomic Modification of an Endogenous Genomic Target of PDCD-1 Gene

In this example, a human PDCD-1 gene was modified using RITDM to eliminate functional PDCD-1 expression in mammalian cells by introducing a stop codon. PDCD-1 encodes programmed cell death protein 1 (PD-1) which has an important role in eliciting an immune checkpoint response of T cells. Tumor cells can be capable of evading immune surveillance and being highly resistant to traditional chemotherapy by activating PD-1. Activation of PD-1 mediated signaling pathway in T cells can lead to decreased activation a number key transcription factors to antagonize positive signals of driving T cell activation, proliferation, effector functions and survival. Blockade of PD-1 signaling in T cells benefits T cell function and survival and can enhance their anti-cancer functionality (Wu et al., Comput Struct Biotechnol J. 2019; 17: 661-674, which is herein incorporated by reference in its entirety). This example was aimed at using RITDM with specifically designed DLR molecules in combination with specific templates to introduce a stop codon in a 5′ region of exon 1 of a PDCD-1 gene to create a strongly truncated translational product and thereby abolish PD-1 signaling cascade in T-cells and boost its anti-cancer therapeutic function.

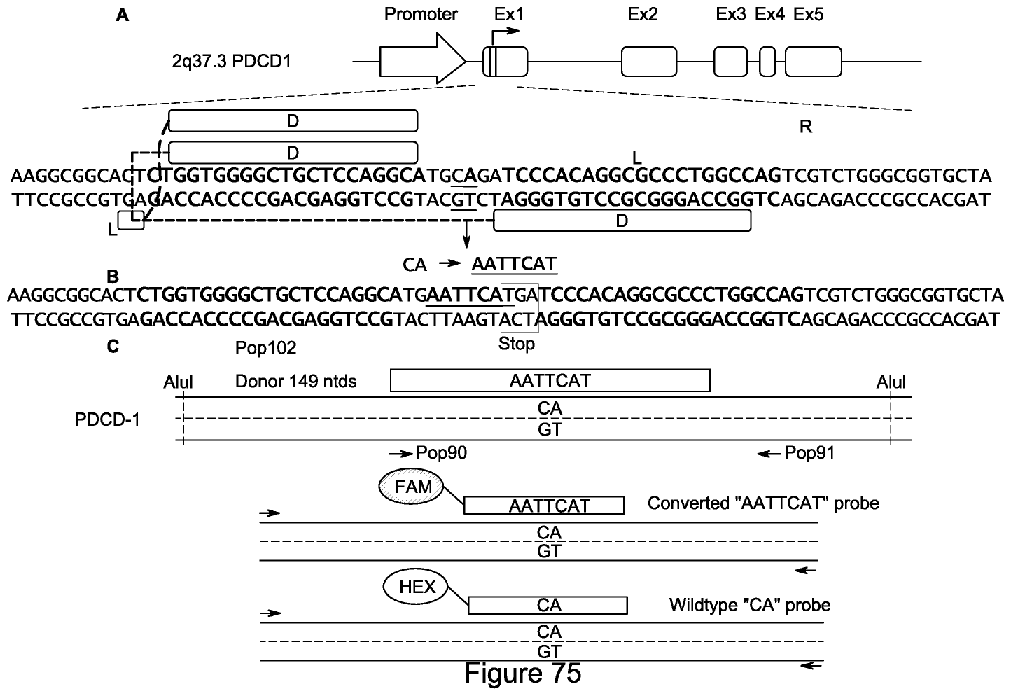

FIG. 75A illustrates an editing strategy used in this example to edit a PDCD-1 gene in U937 cells. In this example, three DLR molecules, encoded on plasmids pb52, pb53 and pb54 (represented by SEQ ID NOS.179-187, which provide DNA and polypeptide sequences) were developed. Pb52 comprises two sequence-specific domains as D- and R-modules, connected with a linker. Both domains comprised 7 zinc-finger arrays each designed to recognize a 21-nucleotide sequence of 5′-CTG-GTG-GGG-CTG-CTC-CAG-GCA (SEQ ID NO.188) respectively 5′-CTG-GCC-AGG-GCG-CCT-GTG-GGA (SEQ ID NO. 189) located on leading respectively lagging strand adjacent to a start codon, “ATG.” Both pb53 and pb54 were designed using a non-sequence specific DNA binding R-domain. The D domain from pb53 was designed to recognize a 21-nucleotide sequence of 5′-CTG-GTG-GGG-CTG-CTC-CAG-GCA (SEQ ID NO.188) on the leading strand of the targeted gene region, utilizing a 7-zinc-finger array. Likewise, the pb54 was designed to recognize a 21-nucleotide sequence of 5′-CTG-GCC-AGG-GCG-CCT-GTG-GGA (SEQ ID NO.189) on the lagging strand, utilizing a 7 zinc-finger array. In this embodiment, illustrated in FIG. 75B a sequence modification polynucleotide with a sequence of 5′TTTCCCTTCCGCTCACCTCCGCCTGAGCAGTGGAGAAGGCGGCACTCTGGTGGGGC TGCTCCAGGCATGAATTCATGATCCCACAGGCGCCCTGGCCAGTCGTCTGGGCGGT GCTACAACTGGGCTGGCGGCCAGGATGGTTCTTAGGT3′ (SEQ ID NO. 190) was used. This was a 149-nucleotide sequence modification polynucleotide with substitution sequence of “AATTCAT” that was intended to replace “CA” at its targeting locus, leading to a stop codon, TGA, in frame. A ddPCR detection strategy is illustrated in FIG. 75C. A relative position of a sequence modification polynucleotide and binding positions of a common primer pair POP90 (SEQ ID NO.191) and POP91 (SEQ ID NO.192) are also indicated. A common primer POP90 locates inside this sequence modification polynucleotide, while POP91 resides outside. Allele-specific probes conjugated with fluorophores FAM and HEX were designed to distinguish between “AATTCAT” and “CA” respectively. AluI restriction enzyme sites are indicated and were used for preparations for ddPCR reactions.

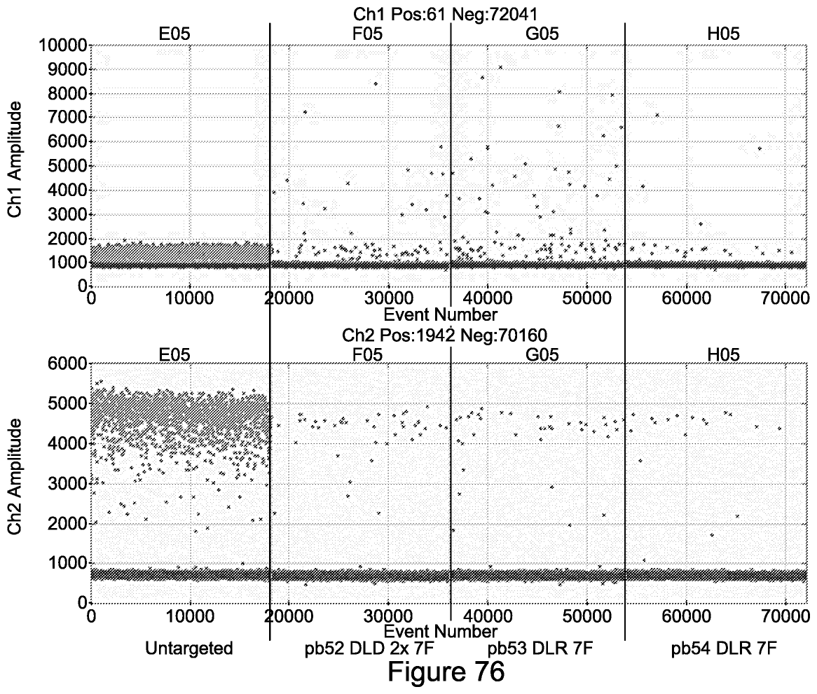

FIG. 76 illustrates successful CA→AATTCAT genetic conversion at a target site in human PDCD-1 as measured by ddPCR. In this example, after transfection, U937 cells were allowed to recover and grow on complete RPMI 1640 medium with 10% FBS for seven days. After five days genomic DNA was isolated and used in digital droplet PCR analysis to determine presence of nucleotide sequences “AATTCAT” or “CA” at PDCD-1. Droplet data is shown in FIG. 76 where “AATTCAT” droplets are displayed in the top panel, while “CA” droplets are displayed in the lower panel. Lane E05 represents no DNA input as negative control, showing neither “AATTCAT” nor “CA” droplets. Lane F05, G05, and H05, represent U937 cells after editing with pb52, pb53 respectively pb54. After RITDM targeting, all three DLRs generated “AATTCAT” droplets, demonstrating that, after being targeted and edited by DLR molecules, in combination with provided sequence modification polynucleotides, successful CA→AATTCAT genetic conversion at human PDCD-1 occurred.

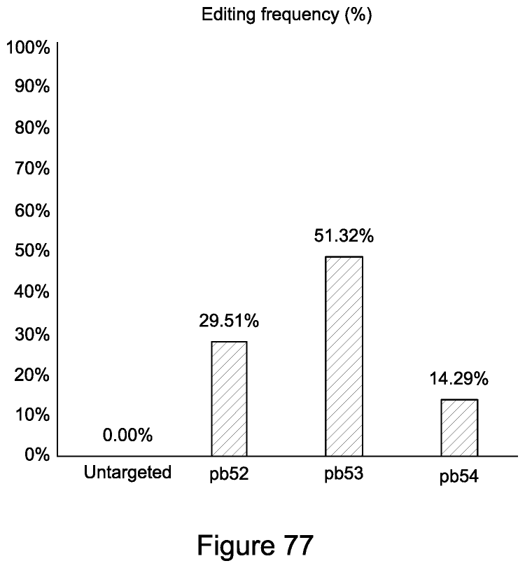

FIG. 77 shows CA→AATTCAT gene conversion frequencies measured by ddPCR after this DLR-based gene editing. Editing frequency in U937 cells were 29.51% with pb52, 51.32% with pb53, and 14.29% with pb54 at the PDCD-1.

### Example 14: Genomic Modification of an Endogenous Genomic Target of CFTR Gene

In this example, a human CFTR (CF transmembrane conductance regulator) gene was modified using RITDM. Loss-of-function mutations in CFTR gene can cause cystic fibrosis which is a common lethal genetic disease. The most prevalent mutation is a deletion of phenylalanine 508 (ΔF508), impairing CFTR folding and, consequently, its biosynthetic and endocytic processing as well as chloride channel function (Lukacs et al., Trends Mol Med. 2012; 18(2): 81-91, which is herein incorporated by reference in its entirety). This example demonstrates use of the RITDM system for gene editing by combining DLR molecules with sequence modification polynucleotides to specifically convert a “CTT” into “ATG” at a position close to codon F508 of CFTR.

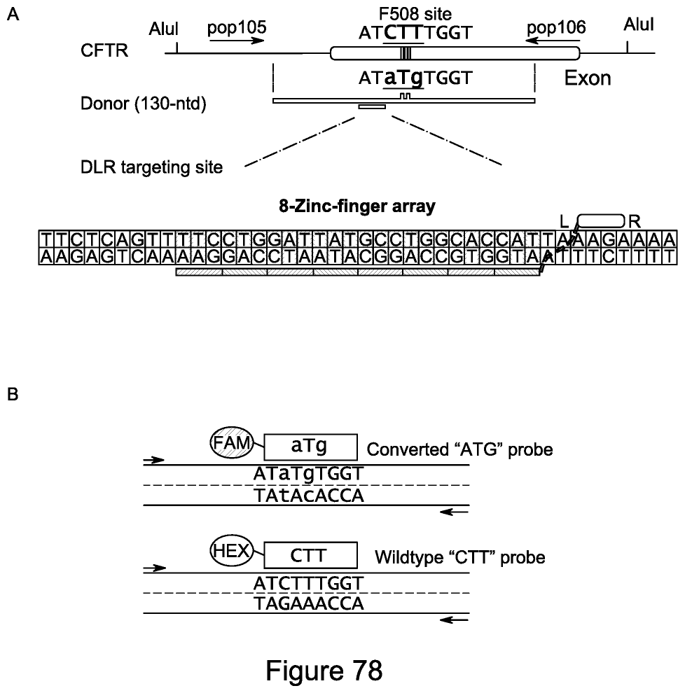

FIG. 78A illustrates an editing strategy used in this example to edit a CFTR gene in HEK293 cells. In this example, a DLR molecule, encoded on plasmid pb64 (represented by SEQ ID NOs.194-196, which provide DNA and polypeptide sequences) was developed. Pb64 comprises a sequence-specific domain as D-element and a non-sequence-specific R-element, connected by a linker (L). This D element comprises an 8-zinc-finger-array designed to recognize a 24-nucleotide sequence of 5′-ATG-GTG-CCA-GGC-ATA-ATC-CAG-GAA (SEQ ID NO.197) located on a lagging strand adjacent to codon F508, “CTT.”

As illustrated in FIG. 78A, a 130 nt sequence modification polynucleotide with a sequence of 5′-GAATTTCATTCTGTTCTCAGTTTTCCTGGATTATGCCTGGCACCATTAAAGAAAATAT CATATGTGGTGTTTCCTATGATGAATATAGATACAGAAGCGTCATCAAAGCATGCCA ACTAGAAGAGGTAAG (SEQ ID NO. 198) was used in this Example. This sequence modification polynucleotide comprises a substitution sequence of “ATG” intended to replace “CTT” at its targeting locus of F508.

HEK293 cells comprising a CFTR gene were contacted by the DLR molecule and sequence specific polynucleotide set forth in SEQ ID NO. 198 as described herein. A ddPCR detection strategy confirmed successful conversion of CTT with ATG at the target site, as depicted in FIG. 78B. Relative positions of a sequence modification polynucleotide and binding positions of a common primer pair POP105 (SEQ ID NO.199) and POP106 (SEQ ID NO.200) are shown in FIG. 78A. A common primer, POP105, binds to a sequence outside of that of the sequence modification polynucleotide used herein, while primer POP106 binds to a sequence inside the sequence modification polynucleotide sequence. Allele-specific probes conjugated with fluorophores FAM and HEX were designed to distinguish between “ATG” and “CTT” respectively. AluI restriction enzyme sites are indicated and were used for preparations for ddPCR reactions.

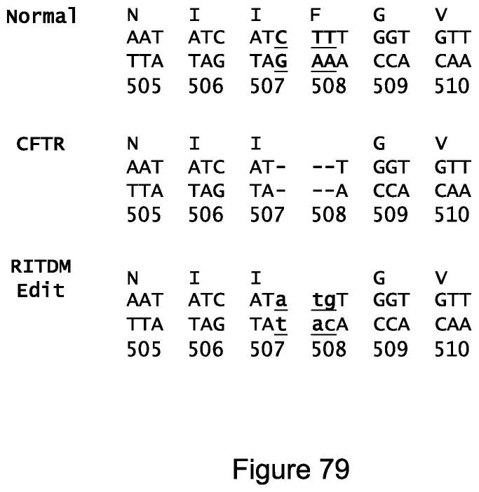

FIG. 79 depicts nucleic acid and amino acid sequences of CFTR adjacent to codon F508 in (i) wild-type (“normal); (ii) CFTR ΔF508, and (iii) predicted sequences after genetic conversion using RITDM editing. A wild-type CFTR amino acid sequence from codons 505 to 510 is NIIFGV (SEQ ID NO. 246). In some cystic fibrosis patients, a deletion of “CTT” can involve a 3^{rd} nucleotide of codon 507, which encodes amino acid isoleucine (I) and a first and second nucleotides of codon 508, which normally encodes phenylalanine (F). Such a deletion results in a third nucleotide, “T” at the codon 508, join two nucleotides “AT” at the previous codon, resulting in an “ATT” triplet; ATT is translated into isoleucine (I). This CTT deletion in cystic fibrosis is termed ΔF508. In this embodiment, nucleotides “CTT” of a CFTR locus in HEK 293 cells was converted to “ATG” to demonstrate successful gene editing at ΔF508 using RITDM.

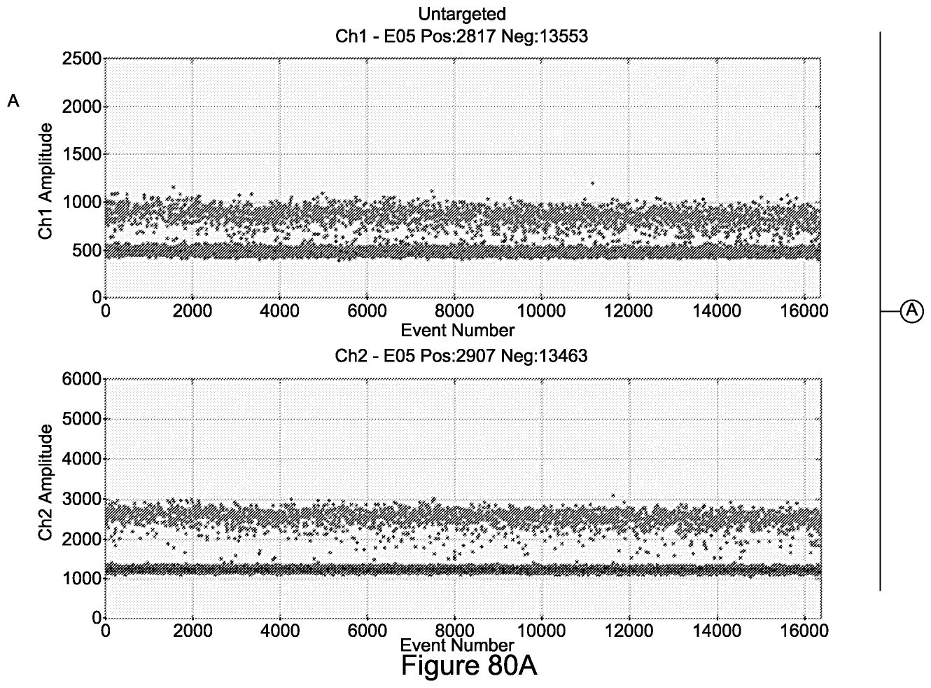

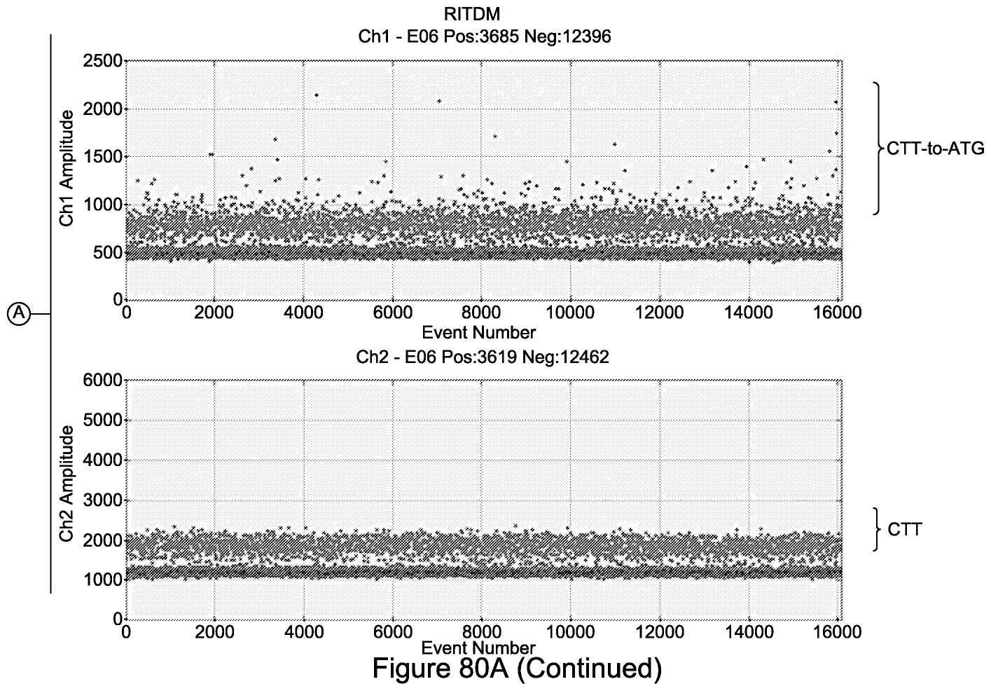

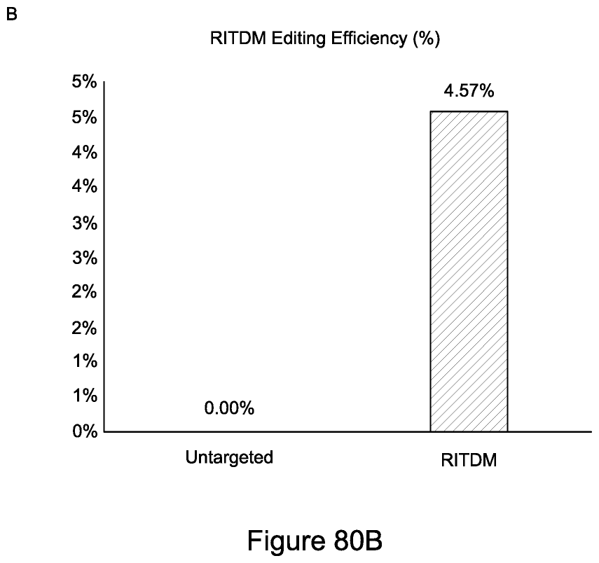

FIGS. 80A and 80B show plots that demonstrate successful CTT→ATG genetic conversion at a target site in human CFTR gene as measured by ddPCR. In this example, after transfection, HEK293 cells were allowed to recover and grow on complete DMEM medium with 10% FBS for five days. After five days genomic DNA was isolated and used in digital droplet PCR analysis to determine presence of nucleotide sequences “ATG” or “CTT” at CFTR1. Raw droplet data are shown in FIG. 80A where edited “ATG” droplets are displayed in the upper panel, while wild type “CTT” droplets are displayed in the lower panel. Untargeted HEK293 cells were used as a negative control and resulted in only wild-type “CTT” droplets with no edited “ATG” droplets detected. After HEK293 cells were transfected with pb64 and sequence modification polynucleotide containing replacement of “ATG” at an equivalent position of “CTT,” ddPCR demonstrated successful targeted conversion of “CTT” into “ATG” at codon F508 site of human CFTR gene. FIG. 80B is a bar graph showing CTT→ATG gene conversion frequencies measured by ddPCR after this DLR-based RITDM gene editing. Editing frequency in targeted HEK293 cells was 4.57% using the pb64 DLR molecule in combination with the sequence modification polynucleotide of SEQ ID NO 198, as compared to 0% in untargeted cells. Thus, RITDM technologies are able to successfully target and gene edit a common cause of a devastating genetic disease without introducing any breaks into genetic material in order to accomplish editing.

Further validation of this “CTT→ATG” conversion was performed, including evaluation of whether any undesired indels were generated. Next generation sequencing of targeted HEK293 pooled cells was performed; untransfected HEK293 cells served as a control. Genomic DNA was isolated and used as a template from which a 154-bp PCR amplicon was generated by using a POP105 and POP106 primer set (as used in the ddPCR analyses in this Example). Amplified PCR products from targeted HEK293 cells and control untransfected (i.e., untargeted) HEK293 cells were analyzed for indels and SNPs on an Illumina next generation sequencing platform (GENEWIZ, South Plainfield, NJ).

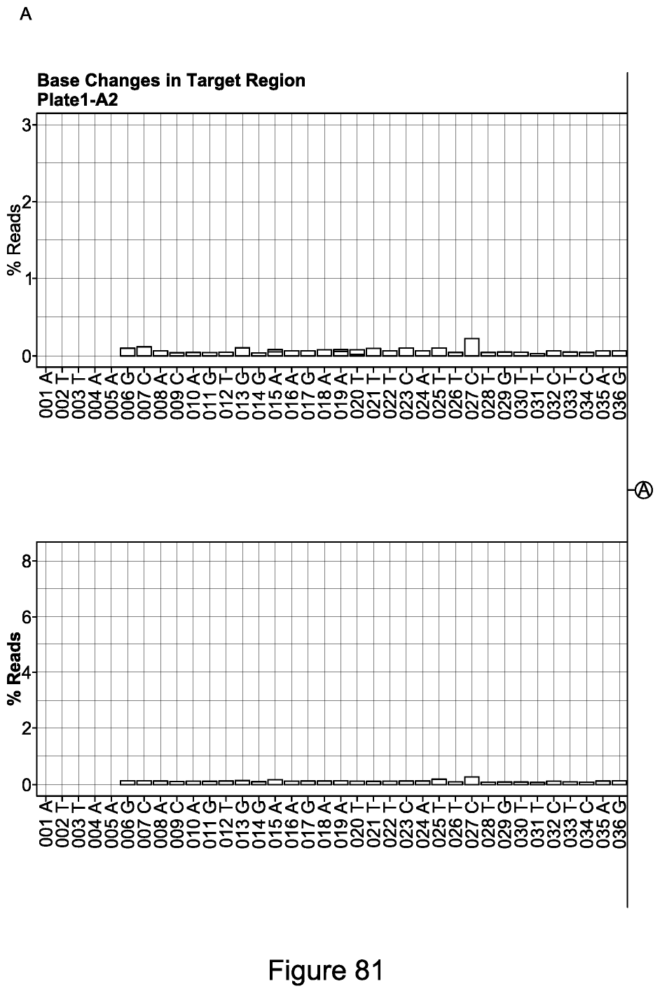

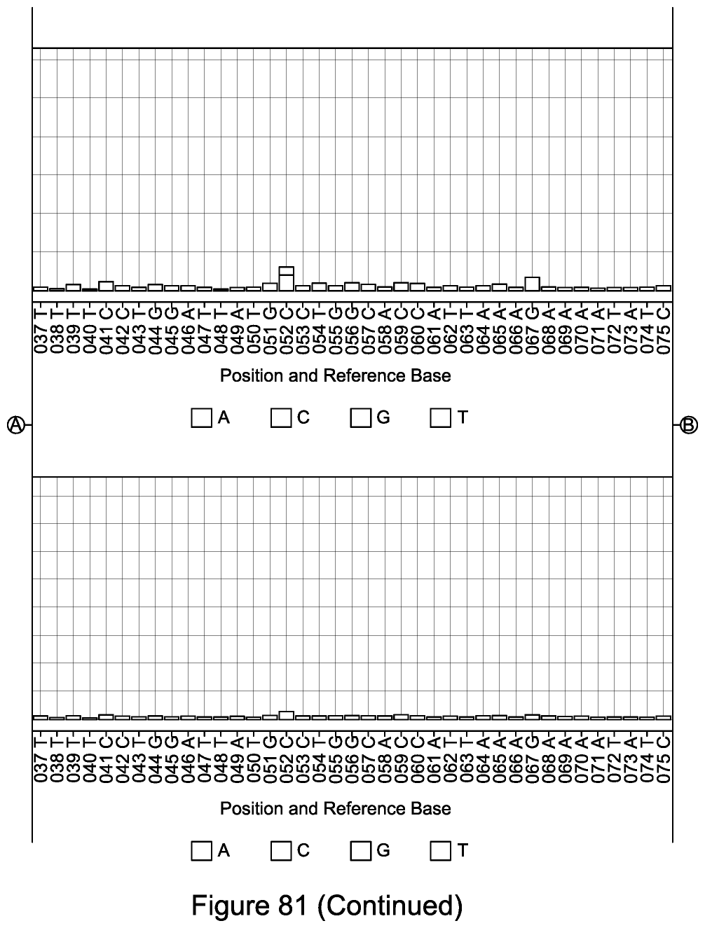

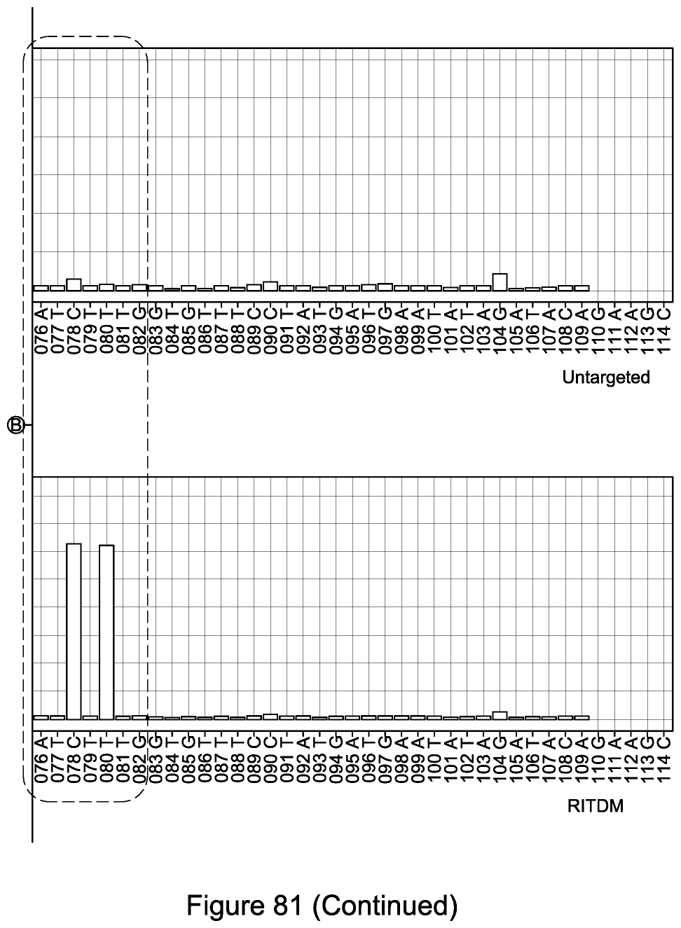

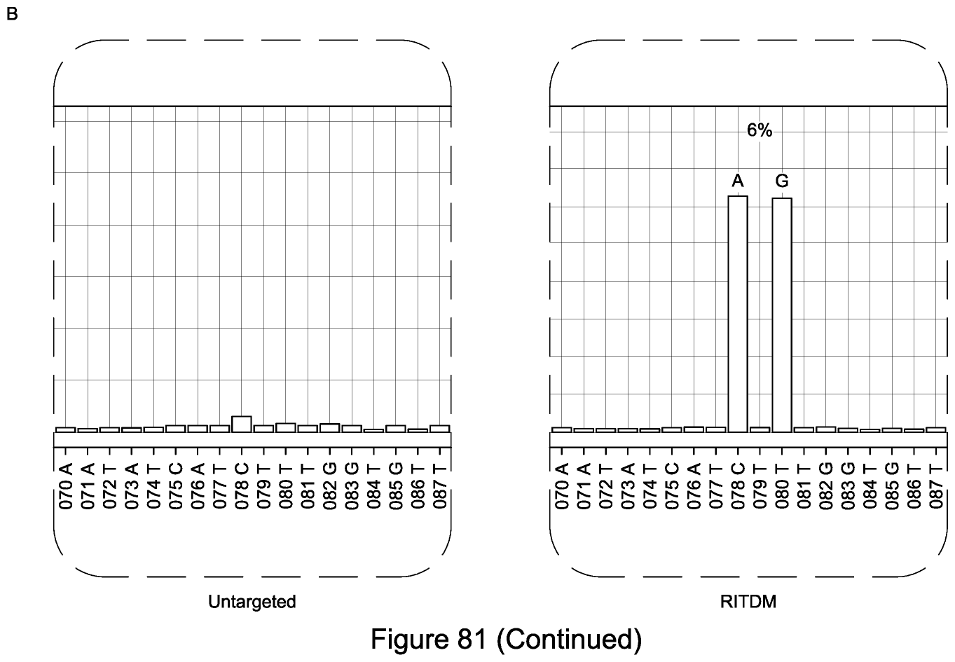

FIG. 81A shows a single nucleotide polymorphisms (SNPs) analysis comparing untargeted and targeted HEK293 cells and confirming detection of genetic conversion of CTT→ATG at the ΔF508 target site, as well as SNPs analysis within a target region of surrounding codon 508 of this CFTR locus. FIG. 81A shows a schematic of an overview of SNPs analysis at these target sites obtained with untargeted and targeted HEK293 pooled cells. Bars represent plotted frequencies of SNPs at each nucleotide position in this 175 bp PCR amplification region. FIG. 81B is a magnified view showing frequencies of CTT→ATG at a target site comparing untargeted and targeted HEK293 cells. As can be seen in the RITDM (i.e., targeted) panel of FIG. 81B, cells transfected with pb64 and a correction template showed a CTT-to-ATG conversion at the target site at a frequency of 6%. Compared to non-transfected HEK293 cells, no other nucleotide conversions occurred at a level significantly above background. A measured frequency of CTT-to-ATG conversion of 6% using NGS analysis was consistent with a rate of 4.57% as determined by ddPCR. Compared to untransfected cells, no unwanted or undesirable SNPs were detected. Average SNP frequencies at other positions in both populations were below 0.5% of total reads. SNPs detected were comparable between these populations and most likely due to background noise in genetic analysis methods. These data again demonstrate that targeting by RITDM did not create significant levels of unintended modifications. Rather, the modifications were specifically and consistently targeted as intended using technologies provided by the RITDM system and the present disclosure.

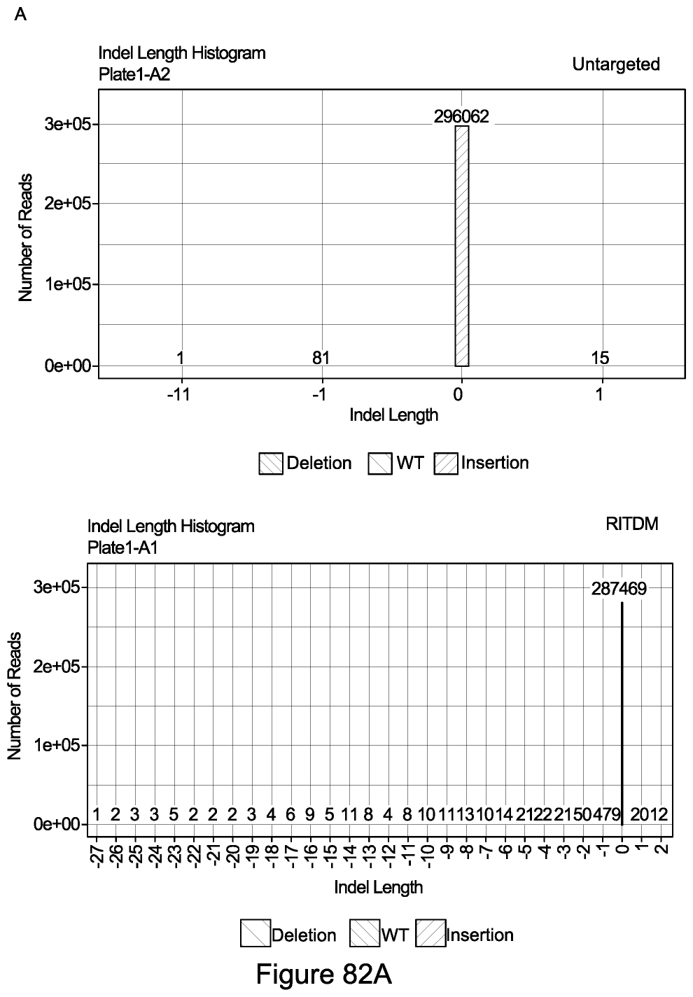

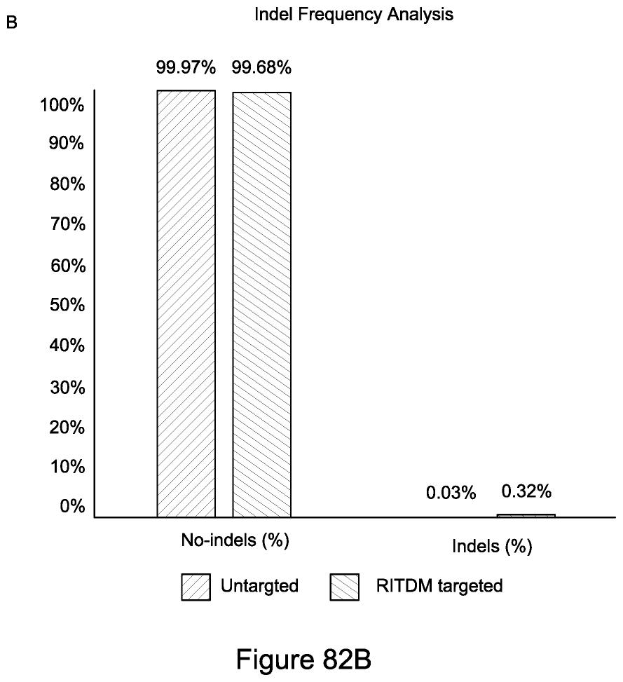

FIGS. 82A and 82B show indel analysis between untargeted and targeted HEK293 pooled cell populations. FIG. 82A shows indel length histograms which plot numbers of deep sequencing reads against a change in length of DNA molecules sequenced. The analysis includes intact sequences (no change in length), insertions and deletions within this targeted amplification region of 154 bp in a human CFTR gene. The left panel of FIG. 82A shows an indel length histogram from untargeted HEK293 cells as a background reference, showing 296062 reads with no change in length; 82 reads contained deletions of one or more nucleotides (81 reads with single nucleotide deletions and 1 read with an 11 nucleotide deletion) and 15 reads had an insertion of one or more nucleotides. The right panel of FIG. 82A shows an indel length histogram from targeted HEK293 cells after RITDM-based gene editing, showing 287469 reads with no change in length; 827 reads contained deletions of one or more nucleic acids (79 single nucleotide deletions, 504 two-nucleotide deletions, and 244 with three or more nucleotide deletions) and 32 reads had an insertion of one or more nucleic acids (20 single nucleotide insertions and 12 two-nucleotide insertions).

FIG. 82B shows indel frequencies calculated as the sum of numbers of sequences with insertions or deletions divided by the total number reads as the sum of numbers of intact, deletion and insertion read, presented as a percentage. In untargeted cells, 99.97% reads were intact and 0.03% contained indels. After RITDM editing, 99.7% reads were intact and only 0.3% had indels.

Collectively, next generation sequencing confirmed and validated successful genetic conversion at the ΔF508 site with very low indel frequencies. These data demonstrate that technologies provided by the present disclosure are capable of accurately changing multiple nucleotides simultaneously in a sequence specific manner at a particular target and target site in a human gene.

### Example 15: Genetic Editing Codon 112 of Human ApoE by dCAS-RITDM

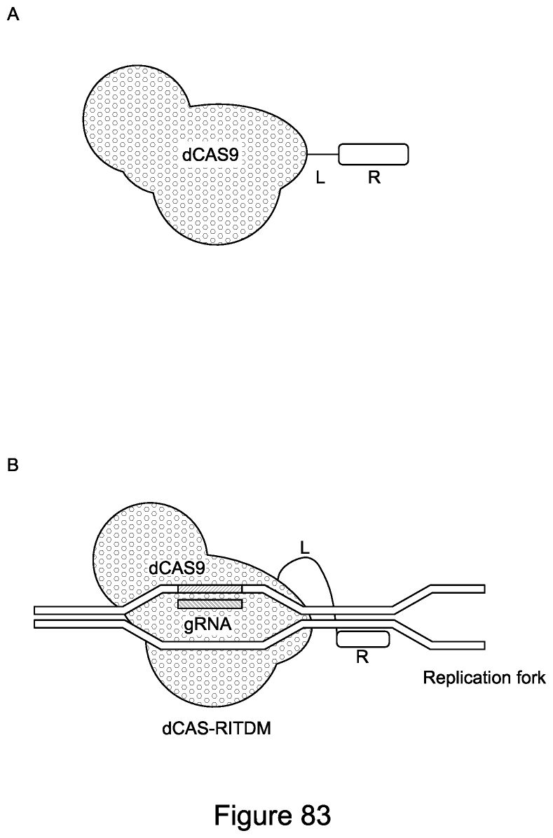

In this Example, codon 112 of a human ApoE gene was modified using RITDM combined with a DLR molecule comprising dCas9, hereinafter referred to as “dCAS-RITDM.” A DLR molecule was designed to use catalytically-inactive Cas9 (dCas9) as a sequence-specific binding motif (i.e., D element). A dCas9 domain was fused to a linker (L element) and an R element. FIG. 83A shows a schematic of an exemplary dCAS-L-R molecule. Since the D element of this DLR molecule is dCas9, it binds to a target site in the presence of a guide RNA as depicted in FIG. 83B.

In this Example, a synthesized guide RNA, POP98-crRNA, 5′-mG*mG*CGCAGGCCCGGCUGGGCGGUUUUAGAGCUAUG*mC*mU-3′ (SEQ ID NO.: 203), annealed with TracrRNA (Genscript, Piscataway, NJ) was designed to target a sequence 5′-GGCGCAGGCCCGGCTGGGCG-3′ (SEQ ID NO.: 204) adjacent to codon 112 of a human ApoE gene. A control guide RNA, ApoE 1112 crRNA2, from a guide RNA supplier (Genscript, Piscataway, NJ), annealed with TracrRNA (Genscript, Piscataway, NJ) was designed to target a sequence 5′-CCTGGTGCAGTACCGCGGCG-3′ (SEQ ID NO.: 205), which is close to codon 112 of a human ApoE gene.

A 129-nucleotide single stranded DNA sequence modification oligonucleotide (i.e., a sequence modification polynucleotide) with a desired T→C substitution roughly located in the middle was used and is set forth as followed with an underlined and bold “C” to for T→C conversion. 5′-CCCCGGTGGCGGAGGAGACGCGGGCACGGCTGTCCAAGGAGCTGCAGGCGGCGCA GGCCCGGCTGGGCGCGGACATGGAGGACGTGCGCGGCCGCCTGGTGCAGTACCGCG GCGAGGTGCAGGCCATGC-3′ (SEQ ID NO.: 22)

Detection of the targeted T→C conversion after DLR-based gene edition were performed by droplet digital PCR (ddPCR). Relative positions of a correction ssODN (i.e., sequence modification polynucleotide) and position of a common primer pair (POP46, POP37, SEQ ID NOS.: 24 and 80) are also indicated in FIG. 17. One common primer, POP46 was located inside this ssODN template (i.e., sequence modification polynucleotide) sequence, while POP37, located outside. Allele-specific probes conjugated with fluorophores FAM and HEX were designed to distinguish between “C” and “T” respectively. PstI restriction enzyme sites indicated were used in preparations for ddPCR reactions.

In this example, a human ApoE gene was edited using dCAS-RITDM which included a DLR molecule comprising a dCas9-based “D” element as described above and herein. The targeted gene conversion was T→C at codon 112 of ApoE and was performed in HEK293 cells. Five days after transfection of the dCas9-L-R-containing plasmid (pb37, SEQ ID NOs.: 63, 64, and 65), guide RNA (SEQ ID NOs.: 203 and 205), and a sequence modification polynucleotide (Pop33, SEQ ID NO.: 22), genomic DNA was extracted and assayed for editing effects by ddPCR. A dCas9 plasmid in presence of a sequence modification polynucleotide and guide RNA was used as a control to demonstrate that dCas9 alone is not capable of induction of genome editing in mammalian cells. The dCas9 is encoded in plasmid pb73 (SEQ ID NO. 206), derived from dCas9-LR plasmid pb37 by removing the region of linker and R-units, containing only catalytically inactive dCas9 cDNA.

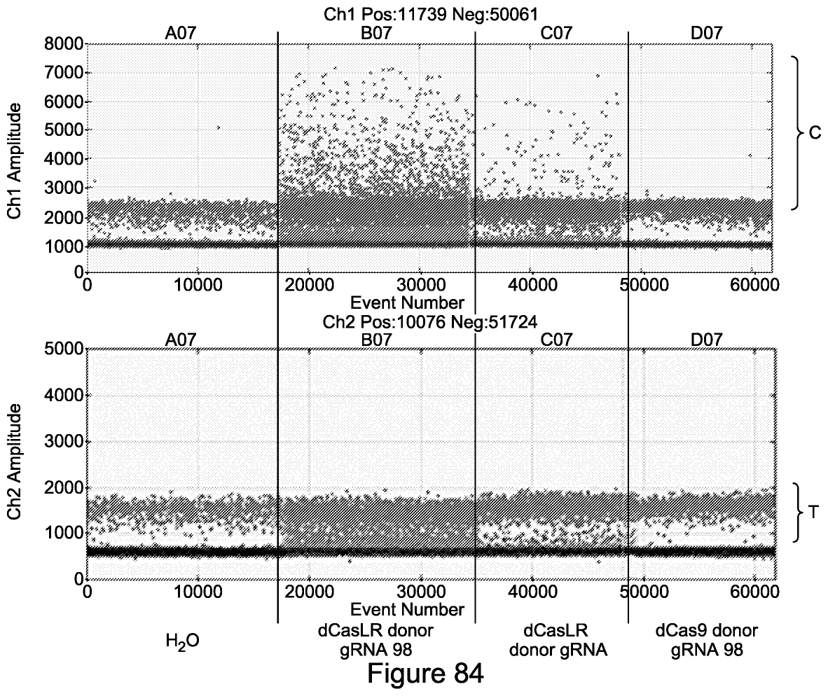

FIG. 84 demonstrates successful T→C conversion at codon 112 of the human ApoE gene in human HEK293 cells, as measured by ddPCR. The upper panel of FIG. 84 shows raw droplet data with “C” droplets; “T” droplets are displayed in the lower panel of FIG. 84. A “no DNA” input was used as negative control, showing neither “C” nor “T” droplets in the lane 1 from the left. The targeted HEK293 cells with dCas9-L-R and sequence modification polynucleotide in combinations with Pop98guide RNA, or a control guide RNA, showed positive “C” droplets, displayed in the lane 2 and 3 from the left. As a control, when using dCas9 instead of dCas9-L-R, very few positive “C” droplets were detected by ddPCR in lane 4 from the right, demonstrating that dCas9 itself, in combination with a sequence modification polynucleotide, but without a DLR molecule cannot result in the targeted gene edit. That is, a DLR molecule is required to achieve the T→C conversion. Collectively, these results demonstrated successful gene editing T→C genetic conversion at codon 112 of human ApoE by using a dCAS-RITDM system comprising a dCas9-based DLR molecule.

Further validation of this T→C conversion was performed, including evaluation of whether any undesired indels were generated. Next generation was performed by next generation sequencing. Next generation sequencing of targeted HEK293 pooled cells was performed; untransfected HEK293 cells served as a control. Genomic DNA was isolated and used as a template from which a 175-bp PCR amplicon was generated by using a POP46 and POP37 primer set (as used in the ddPCR analyses in this Example). Amplified PCR products from targeted HEK293 cells with two guide RNA molecules, and control untransfected (and thus, untargeted) HEK293 cells were analyzed for indels and SNPs on an Illumina next generation sequencing platform (GENEWIZ, South Plainfield, NJ).

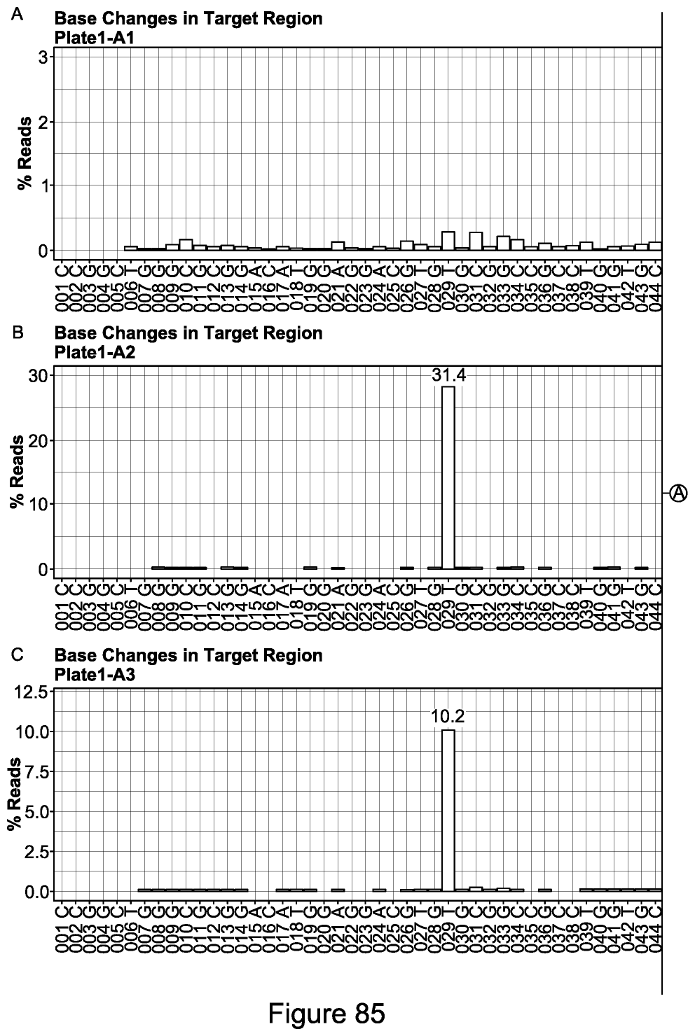

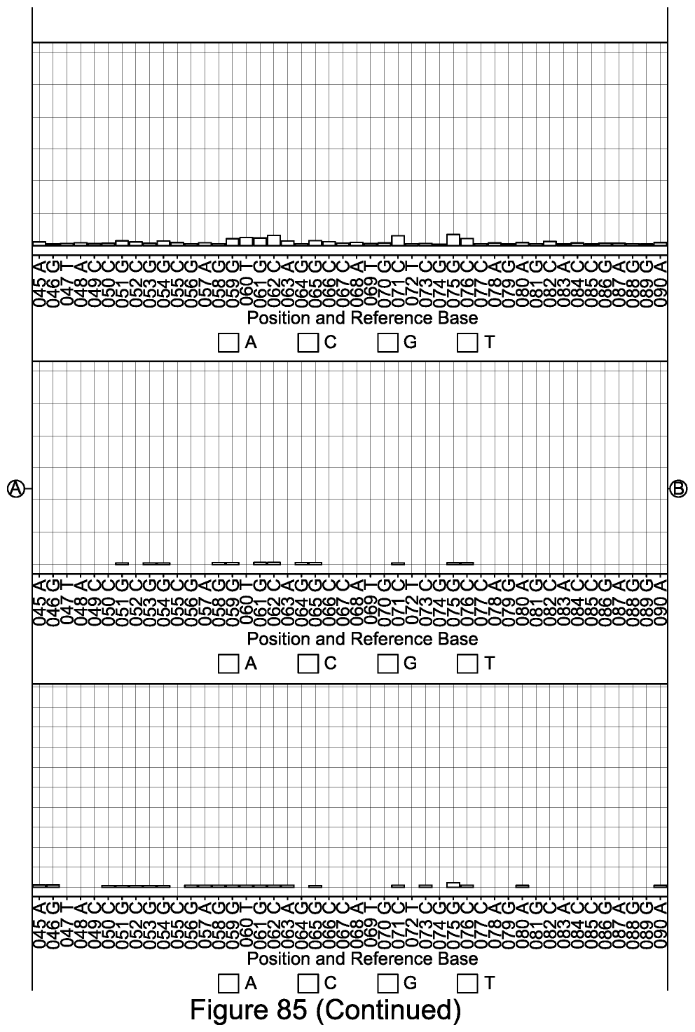

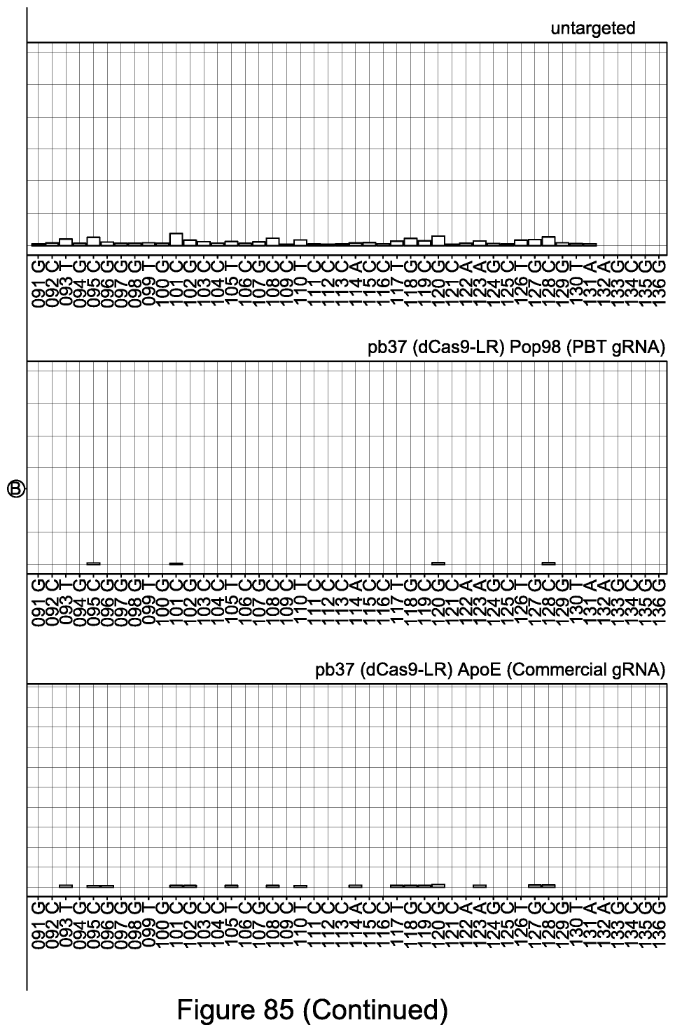

FIG. 85 shows a single nucleotide polymorphisms (SNPs) analysis comparing untargeted and targeted HEK293 cells and confirming detection of genetic conversion of T→C at this target site as well as SNPs analysis within a target region of surrounding codon 112 of this ApoE locus.

FIG. 85A shows an overview of SNPs analysis at these target sites obtained with untargeted HEK293 pooled cells. Bars represent plotted frequencies of SNPs at each nucleotide position in this 175 bp PCR amplification region. FIGS. 85B and 85C show overviews of SNPs analysis at these target sites obtained with targeted HEK293 pooled cells with two guide RNAs. Compared to non-transfected HEK293 cells, using POP98 guide RNA, dCAS-RITDM induced T→C conversion at this expected site with a frequency of 31.4%. When using a commercially available guide RNA a T→C converting frequency of 10.2% was obtained. In both cases no other nucleotide conversions occurred at a level significantly above background. Average SNP frequencies at off-target positions in all three populations were below 0.5% of total reads. SNPs detected were comparable between these populations and most likely due to background noise in genetic analysis methods. These data further demonstrate that targeting by dCAS-RITDM did not create significant levels of unintended modifications.

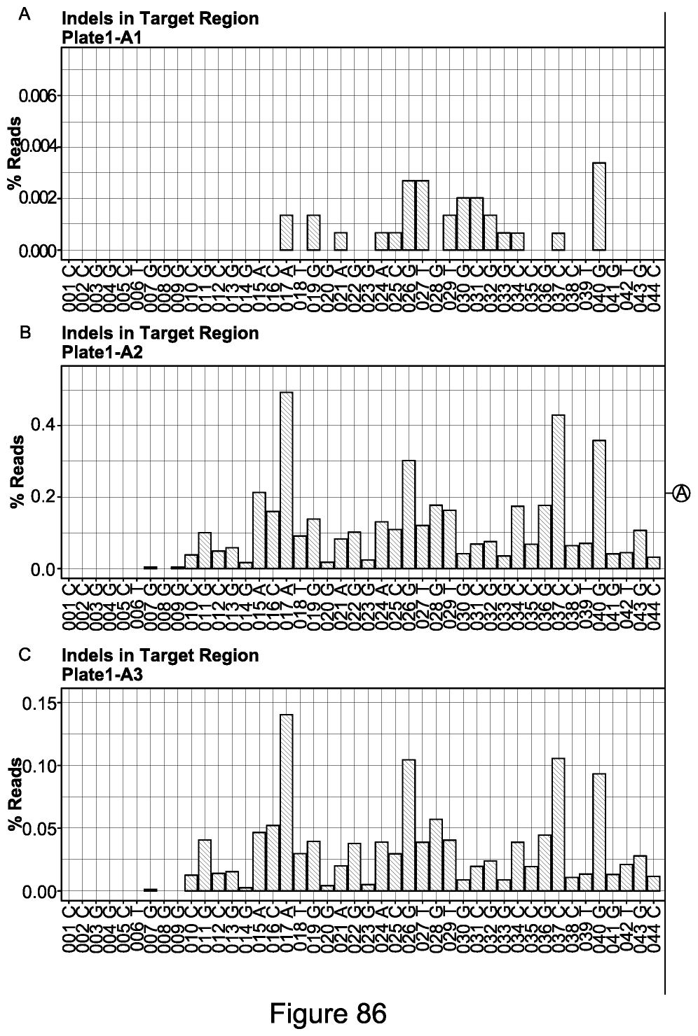

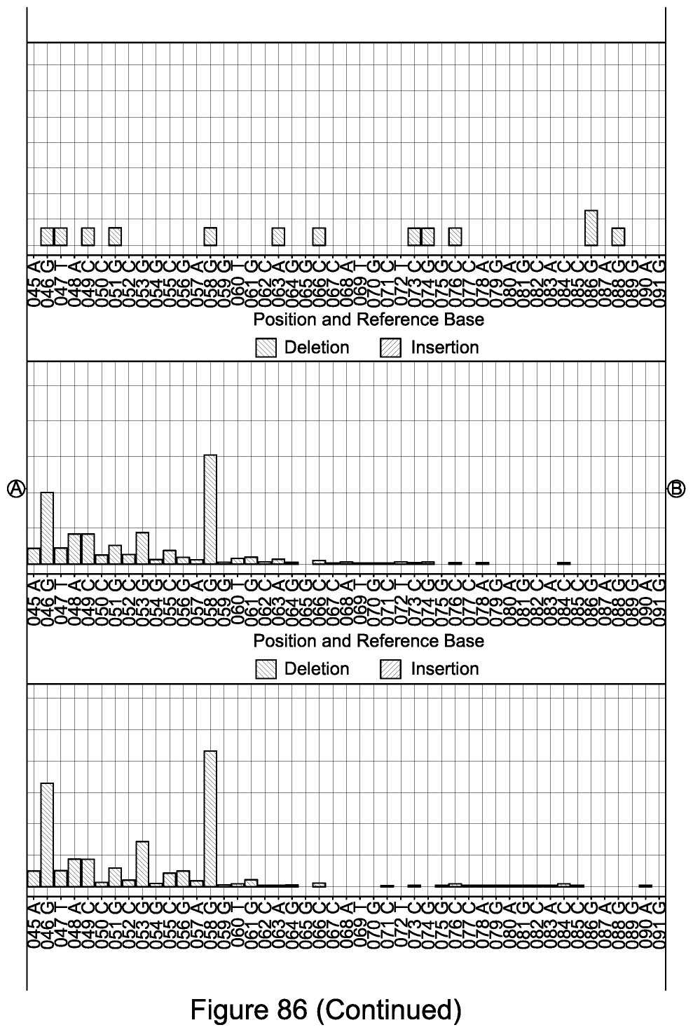

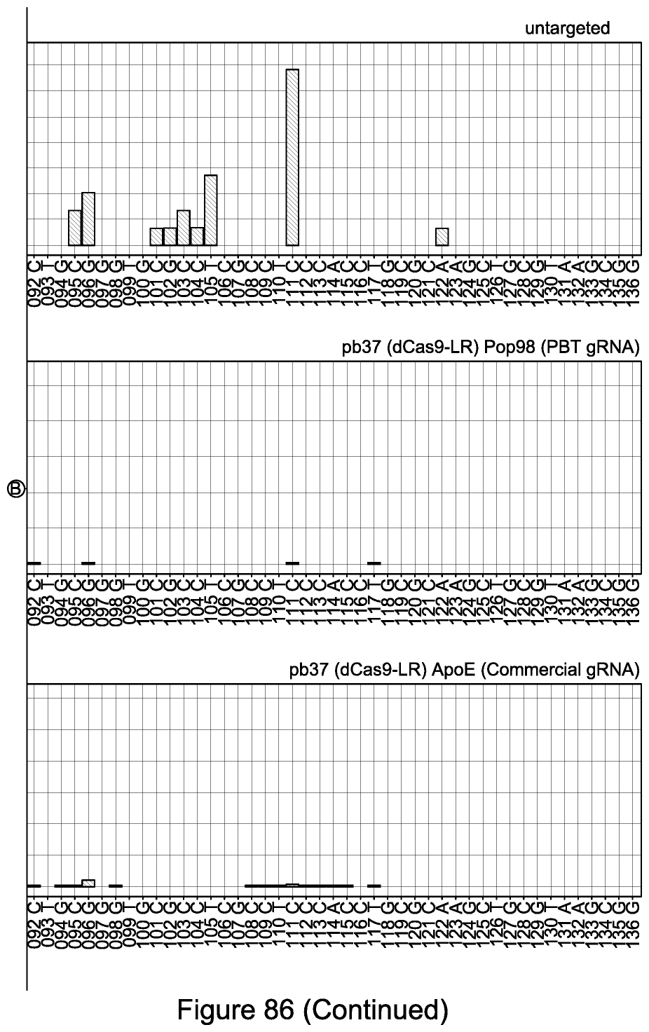

FIG. 86 shows insertion and deletion analysis around codon 112 of ApoE in this example, showing frequency plots of insertions and deletions analysis for untargeted HEK293 cells and targeted pooled HEK293 cells by using dCAS-RITDM. Bars plot frequencies of insertions and deletions at each nucleotide position of this 175 bp PCR amplification region. This indels analysis showed, in general, a very low frequency (<0.5%) of insertions and/or deletions at each position within this 175 bp amplification region in untargeted (FIG. 86A), targeted with Pop98 guide RNA (FIG. 86B), and with a commercially available ApoE guide RNA (FIG. 86C).

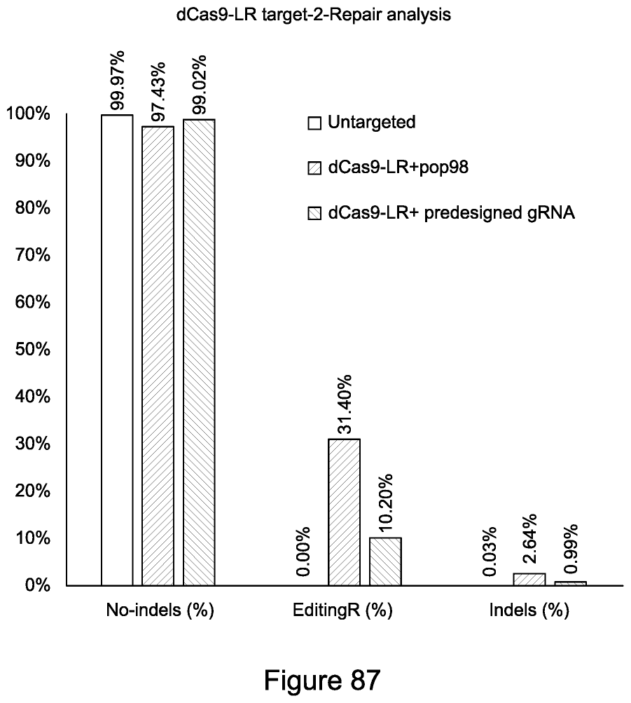

FIG. 87 shows overall editing and indel frequencies calculated based on deep sequencing results. dCAS-RITDM is able to successfully induce T→C conversion with calculated frequencies of approximately 31.4% respectively 10.2% using two different gRNA for targeting, with indel frequencies of 2.64% and 0.99%, respectively.

Collectively, next generation sequencing confirmed and validated successful T→C genetic conversion at codon 112 of ApoE with very low indel frequencies, and demonstrates that technologies as provided herein are capable of inducing accurate and carefully tailored genome editing using dCAS-RITDM comprising a dCas9-based D element.

### Example 16: Transcription Modification Mediated Suppression of Oncogenic KRAS Gene Expression in Mammalian Cells

In this example, human KRAS gene expression was inhibited by programmed gene regulation via DLR molecules. KRAS is a frequent oncogenic driver in solid tumors, including pancreatic cancer, colon cancer, non-small cell lung cancer (NSCLC), and many others (Salgia R. et.al. Cell Rep Med 2021; January 19; 2(1):100186., which is herein incorporated by reference in its entirety). Few treatments are available for targeting KRAS directly, and KRAS mutations are often considered as “undruggable” targets. As demonstrated herein DLR molecules can be used to suppress KRAS gene expression as evidenced by reduced mRNA levels.

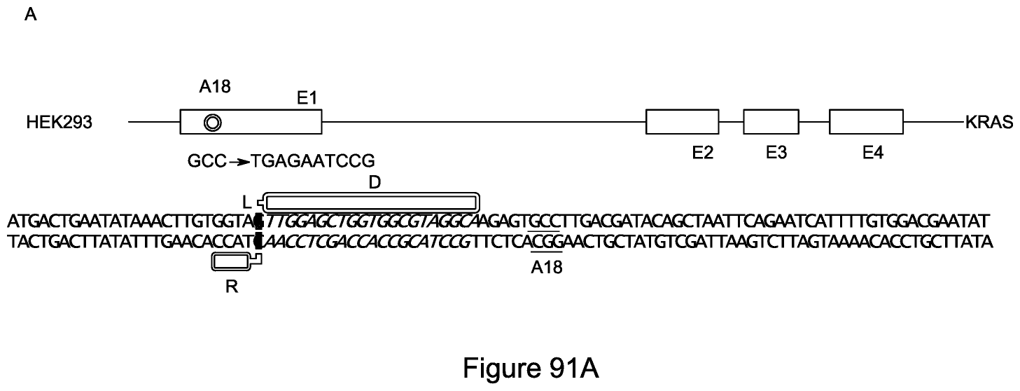

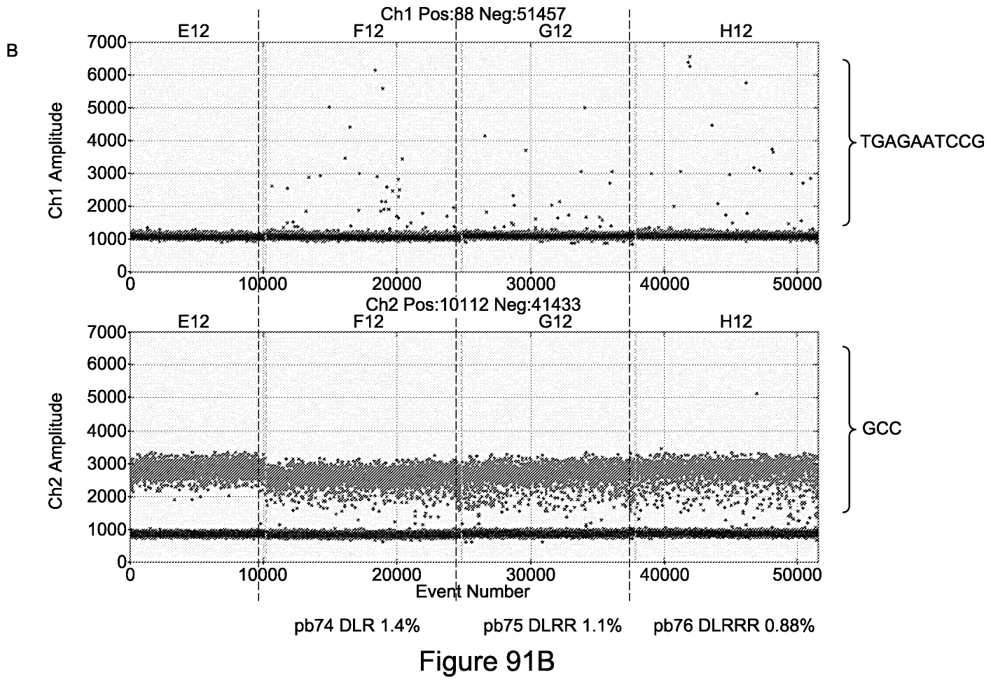

FIG. 91A illustrates an exemplary transcription modification strategy used in this example to target KRAS genes in HEK293 cells with DLR molecules. In this example, three different DLR molecules, encoded on plasmid pb74, pb75, and pb76 (represented by SEQ ID NOs.217-225, for full-length DNA, cDNA, and amino acid sequences) were developed (See exemplary structures in FIG. 90). Sequence-specific D domains comprised a 7-zinc-finger-array designed to recognize a 21-nucleotide sequence of 5′-TTG-GAG-CTG-GTG-GCG-TAG-GCA (SEQ ID NO.226) located on leading strand adjacent to codon A18 “GCC.” within Exon 1.

As exemplary proof of targeting specificity, RITDM was used to confirm KRAS targeting. In this embodiment, a 137 nt sequence modification polynucleotide was first used to confirm targeting and is set forth as follows: 5′-AAAATGACTGAATATAAACTTGTGGTAGTTGGAGCTGGTGGCGTAGGCAAGAGTTG AGAATCCGTTGACGATACAGCTAATTCAGAATCATTTTGTGGACGAATATGATCCAA CAATAGAGGTAAATCTTGTTTTAA-3′ (SEQ ID NO. 227). This sequence modification polynucleotide has a substitution sequence of “TGAGAATCCG” (SEQ ID NO. 241) that was intended to replace “GCC” at its targeting locus of KRAS. Each of plasmid of pb74, pb75, and pb76 along with sequence modification polynucleotide were introduced into HEK 293 cells by electroporation and reseeded into tissue culture vessels. Five days post transfection, genomic DNA were extracted, followed by ddPCR detection for genome editing effects. As shown in FIG. 91B, ddPCR analysis demonstrates successful KRAS targeting. The upper panel of FIG. 91B represents positive droplets with “TGAGAATCCG” (SEQ ID NO. 241) genetic conversion; the lower panel of FIG. 91B represents wild type droplets comprising “GCC.” All three DLR molecules with single (DLR), double (DLRR), or triple R (DLRRR) elements, were able to successfully convert “GTT” into “TGAGAATCCG” (SEQ ID NO. 241) at target site of KRAS gene in human genome in HEK293 cells, demonstrating that these DLR molecules are able to accurately target a human KRAS gene sequence. This also confirms site-specific binding of each of these DLR molecules as designed.

Next, programmed KRAS gene suppression was performed and analyzed. In HEK293 cells, each of plasmids, pb74 (i.e., DLR), pb75 (i.e., DLRR), or pb76 (i.e., DLRRR) was introduced into cells by electroporation. A “no DNA” transfection was used as control. Seventy-hours post electroporation, cells transfected with each plasmid were detached and collected. Total RNAs from each condition were then extracted by using Trizol reagent. Five hundred ng of total RNA was then converted into DNA by reverse transcription (RT) using a reverse transcriptase, corresponding buffer, and dNTPs. After this RT reaction, a PCR test was conducted using a primer set of Pop 133 (SEQ ID. NO. 228) and Pop134 (SEQ ID. NO. 229).

As illustrated in FIG. 92A, primer Pop 133 is a forward primer binding within Exon1 of the human KRAS gene; and Pop134 is a reverse one binding on Exon2 of human KRAS gene. When KRAS mRNA was present, a 184 bp RT-PCR amplicon was detected. FIG. 92B shows successful suppression of KRAS gene expression by pb74 (DLR), pb75 (DLRR), and pb76 (DLRRR). In each condition, RT-PCR conducted using a primer set of Pop133 and Pop134 showed RT-PCR amplicons of 184 bp in length, which is the same size as a positive control. After transfection pb74, pb75, and pb76, intensity of all three RT-PCR bands was weaker than the control condition. The reference (ref-BMG) was generated by performing RT-PCR reaction for a house-keeping gene beta-microglobin (BMG), which can be used for quantitation and normalization of each condition. These results demonstrate that KRAS gene expression was suppressed by all three DLR molecule designs. Collectively this illustrates that DLR molecules can be used to successfully perform targeted, programmed gene suppression.

FIG. 93 shows quantitation of programmed gene regulation using pb74 (DLR), pb75 (DLRR), and pb76 (DLRRR) in U937 cells. As described above, each plasmid, pb74, pb75, and pb76 was introduced into U937 cells by electroporation. A “no DNA” transfection was used as control. Seventy-hours post electroporation, cells transfected with these plasmids were detached and collected. Total RNAs from each condition were then extracted by using Trizol reagent. Five hundred nanograms of total RNA was then converted into DNA by reverse transcription (RT) reaction, followed by PCR using a primer set of Pop133 (SEQ ID. NO. 228) and Pop134 (SEQ ID. NO. 229). Three independent experiments were conducted. KRAS mRNA expression was quantitated by calculations of amplification band intensity of RT-PCR KRAS normalized by corresponding that of Ref-BMG using Bio-Rad Imagelab software. Introduction of pb74 (DLR), ob75 (DLRR), and pb76 (DLRRR) inhibit KRAS gene expression more than 50%. Collectively these results further illustrate that DLR molecules can successfully performed targeted, programmed gene suppression.

TABLE 1
Sequences

| | | | Sequence (5′-3′) or |
|---|---|---|---|
| | | | (N-C term) (* represents |
| SEQ ID # | Type | Brief description | stop codon) |
| SEQ ID | Amino | Linker | LRGS |
| NO. 1 | Acid | | |
| SEQ ID | Amino | Zinc finger frame 1 | FQCRICMRNFS(X7)HIRTH |
| NO. 2 | Acid | | |
| SEQ ID | Amino | Zinc finger frame 2 | FACDICGRKFA(X7)HTKIH |
| NO. 3 | Acid | | |
| SEQ ID | DNA | EGFPDP2 DLR | GGGGAGGACGCGGTG |
| NO. 4 | | targeting site (1) | |
| SEQ ID | Amino | EGFPDP2 DLR D | FQCRICMRNFSRSSALTRHIRTHTGEKPFACDI |
| NO. 5 | Acid | element 5-zinc- | CGRKFARSDTLTRHTKIHTGSQKPFQCRICMRN |
| | | finger array | FSDRSNLTRHIRTHTGEKPFACDICGRKFARSD |
| | | | NLTRHTKIHTGSQKPFQCRICMRNFSRSDHLTR |
| | | | HIRTHTG |
| SEQ ID | DNA | EGFPDP2 DLR | GTGGAGCTGGACGGGGAC |
| NO. 6 | | targeting site (2) | |
| SEQ ID | Amino | EGFPDP2 DLR R | FQCRICMRNFSDRSNLTRHIRTHTGEKPFACDI |
| NO. 7 | Acid | element 6-zinc- | CGRKFARSDHLTRHTKIHTGSQKPFQCRICMRN |
| | | finger array | FSDRSNLTRHIRTHTGEKPFACDICGRKFARSD |
| | | | SLSEHTKIHTGSQKPFQCRICMRNFSRSSNLTR |
| | | | HIRTHTGEKPFACDICGRKFARSDSLTRHTKIH |
| SEQ ID | DNA | ApoE codon 112 | GCGGCCGCCTGGTGCAGTACCGCGGCG |
| NO. 8 | | site DLR targeting | |
| | | site | |
| SEQ ID | Amino | ApoE codon 112 | MAAMAERPFQCRICMRNFSRSSDLTRHIRTHTG |
| NO. 9 | Acid | site DLR D element | EKPFACDICGRKFARSDTLTRHTKIHTGSQKPF |
| | | 9-zinc-finger array | QCRICMRNFSQSGDLSEHIRTHTGEKPFACDIC |
| | | | GRKFATSGHLTTHTKIHTGSQKPFQCRICMRNF |
| | | | SDSSHLTTHIRTHTGEKPFACDICGRKFARSSH |
| | | | LTTHTKIHTGSQKPFQCRICMRNFSDRSDLTRH |
| | | | IRTHTGEKPFACDICGRKFADRSDLTRHTKIHT |
| | | | GSQKPFQCRICMRNFSRSDTLTRHIRTHTG |
| SEQ ID | DNA | ApoE codon 158 | CTGGCAGTGTACCAGGCCGGGGCCCGCGAGGGC |
| NO. 10 | | site DLR targeting | |
| | | site | |
| SEQ ID | Amino | ApoE codon 158 | MAAMAERPFQCRICMRNFSDRSHLTRHIRTHTG |
| NO. 11 | Acid | site DLR D element | EKPFACDICGRKFARSDNLTRHTKIHTGSQKPF |
| | | 11-zinc-finger array | QCRICMRNFSDSSHLSEHIRTHTGEKPFACDIC |
| | | | GRKFADRSDLTRHTKIHTGSQKPFQCRICMRNF |
| | | | SRSDHLTRHIRTHTGEKPFACDICGRKFADRSD |
| | | | LTRHTKIHTGSQKPFQCRICMRNFSRSDNLSEH |
| | | | IRTHTGEKPFACDICGRKFAESSNLTTHTKIHT |
| | | | GSQKPFQCRICMRNFSRSSSLTRHIRTHTGEKP |
| | | | FACDICGRKFAQSSDLTRHTKIHTGSQKPFQCR |
| | | | ICMRNFSRSDSLSEHIRTHTG |
| SEQ ID | Amino | dcas9 | DKKYSIGLAIGTNSVGWAVITDEYKVPSKKFKV |
| NO. 12 | Acid | | LGNTDRHSIKKNLIGALLFDSGETAEATRLKRT |
| | | | ARRRYTRRKNRICYLQEIFSNEMAKVDDSFFHR |
| | | | LEESFLVEEDKKHERHPIFGNIVDEVAYHEKYP |
| | | | TIYHLRKKLVDSTDKADLRLIYLALAHMIKERG |
| | | | HFLIEGDLNPDNSDVDKLFIQLVQTYNQLFEEN |
| | | | PINASGVDAKAILSARLSKSRRLENLIAQLPGE |
| | | | KKNGLFGNLIALSLGLTPNFKSNEDLAEDAKLQ |
| | | | LSKDTYDDDLDNLLAQIGDQYADLFLAAKNLSD |
| | | | AILLSDILRVNTEITKAPLSASMIKRYDEHHQD |
| | | | LTLLKALVRQQLPEKYKEIFFDQSKNGYAGYID |
| | | | GGASQEEFYKFIKPILEKMDGTEELLVKLNRED |
| | | | LLRKQRTFDNGSIPHQIHLGELHAILRRQEDFY |
| | | | PFLKDNREKIEKILTFRIPYYVGPLARGNSRFA |
| | | | WMTRKSEETITPWNFEEVVDKGASAQSFIERMT |
| | | | NFDKNLPNEKVLPKHSLLYEYFTVYNELTKVKY |
| | | | VTEGMRKPAFLSGEQKKAIVDLLFKTNRKVTVK |
| | | | QLKEDYFKKIECFDSVEISGVEDRFNASLGTYH |
| | | | DLLKIIKDKDFLDNEENEDILEDIVLTLTLFED |
| | | | REMIEERLKTYAHLFDDKVMKQLKRRRYTGWGR |
| | | | LSRKLINGIRDKQSGKTILDELKSDGFANRNEM |
| | | | QLIHDDSLTFKEDIQKAQVSGQGDSLHEHIANL |
| | | | AGSPAIKKGILQTVKVVDELVKVMGRHKPENIV |
| | | | IEMARENQTTQKGQKNSRERMKRIEEGIKELGS |
| | | | QILKEHPVENTQLQNEKLYLYYLQNGRDMYVDQ |
| | | | ELDINRLSDYDVDAIVPQSFLKDDSIDNKVLTR |
| | | | SDKNRGKSDNVPSEEVVKKMKNYWRQLLNAKLI |
| | | | TQRKFDNLTKAERGGLSELDKAGFIKRQLVETR |
| | | | QITKHVAQILDSRMNTKYDENDKLIREVKVITL |
| | | | KSKLVSDFRKDFQFYKVREINNYHHAHDAYLNA |
| | | | VVGTALIKKYPKLESEFVYGDYKVYDVRKMIAK |
| | | | SEQEIGKATAKYFFYSNIMNFFKTEITLANGEI |
| | | | RKRPLIETNGETGEIVWDKGRDFATVRKVLSMP |
| | | | QVNIVKKTEVQTGGFSKESILPKRNSDKLIARK |
| | | | KDWDPKKYGGFDSPTVAYSVLVVAKVEKGKSKK |
| | | | LKSVKELLGITIMERSSFEKNPIDFLEAKGYKE |
| | | | VKKDLIIKLPKYSLFELENGRKRMLASAGELQK |
| | | | GNELALPSKYVNFLYLASHYEKLKGSPEDNEQK |
| | | | QLFVEQHKHYLDEIIEQISEFSKRVILADANLD |
| | | | KVLSAYNKHRDKPIREQAENIIHLFTLTNLGAP |
| | | | AAFKYFDTTIDRKRYTSTKEVLDATLIHQSITG |
| | | | LYETRIDLSQLGGD |
| SEQ ID | Amino | Linker for dCas9 | LRQKDAARGS |
| NO. 13 | Acid | based DLR | |
| SEQ ID | Amino | longer linker for | GGGGGSGGGGGSGGGGGSGGGGGSGGGGGSGGG |
| NO. 14 | Acid | DLR, featuring dual | GGS |
| | | zinc finger arrays | |
| SEQ ID | Amino | EGFPDP2 | MVSKGEELFTASSPSSWSWTGT* |
| NO. 15 | Acid | | |
| SEQ ID | Amino | EGFPD | MVSKGEELFTGVVPILVELDGDVNGHKFSVSGE |
| NO. 16 | Acid | | GEGDATYGKLTLKFICTTGKLPVPWPTLVTTLT |
| | | | YGVQCFSRYPDHMKQHDFFKSAMPEGYVQERTI |
| | | | FFKDDGNYKTRAEVKFEGDTLVNRIELKGIDFK |
| | | | EDGNILGHKLEYNYNSHNVYIMADKQKNGIKVN |
| | | | FKIRHNIEDGSVQLADHYQQNTPIGDGPVLLPD |
| | | | NHYLSTQSALSKDPNEKRDHMVLLEFVTAAGIT |
| | | | LGMDELYK* |
| SEQ ID | DNA | pcDNA5/FRT/EGF | GACGGATCGGGAGATCTCCCGATCCCCTATGGT |
| NO. 17 | | PDP2 | GCACTCTCAGTACAATCTGCTCTGATGCCGCAT |
| | | | AGTTAAGCCAGTATCTGCTCCCTGCTTGTGTGT |
| | | | TGGAGGTCGCTGAGTAGTGCGCGAGCAAAATTT |
| | | | AAGCTACAACAAGGCAAGGCTTGACCGACAATT |
| | | | GCATGAAGAATCTGCTTAGGGTTAGGCGTTTTG |
| | | | CGCTGCTTCGCGATGTACGGGCCAGATATACGC |
| | | | GTTGACATTGATTATTGACTAGTTATTAATAGT |
| | | | AATCAATTACGGGGTCATTAGTTCATAGCCCAT |
| | | | ATATGGAGTTCCGCGTTACATAACTTACGGTAA |
| | | | ATGGCCCGCCTGGCTGACCGCCCAACGACCCCC |
| | | | GCCCATTGACGTCAATAATGACGTATGTTCCCA |
| | | | TAGTAACGCCAATAGGGACTTTCCATTGACGTC |
| | | | AATGGGTGGAGTATTTACGGTAAACTGCCCACT |
| | | | TGGCAGTACATCAAGTGTATCATATGCCAAGTA |
| | | | CGCCCCCTATTGACGTCAATGACGGTAAATGGC |
| | | | CCGCCTGGCATTATGCCCAGTACATGACCTTAT |
| | | | GGGACTTTCCTACTTGGCAGTACATCTACGTAT |
| | | | TAGTCATCGCTATTACCATGGTGATGCGGTTTT |
| | | | GGCAGTACATCAATGGGCGTGGATAGCGGTTTG |
| | | | ACTCACGGGGATTTCCAAGTCTCCACCCCATTG |
| | | | ACGTCAATGGGAGTTTGTTTTGGCACCAAAATC |
| | | | AACGGGACTTTCCAAAATGTCGTAACAACTCCG |
| | | | CCCCATTGACGCAAATGGGCGGTAGGCGTGTAC |
| | | | GGTGGGAGGTCTATATAAGCAGAGCTCTCTGGC |
| | | | TAACTAGAGAACCCACTGCTTACTGGCTTATCG |
| | | | AAATTAATACGACTCACTATAGGGAGACCCAAG |
| | | | CTGGCTAGCGTTTAAACTTAAGCTTATGGTGAG |
| | | | CAAGGGCGAGGAGCTGTTCACCGCGTCCTCCCC |
| | | | ATCCTCGTGGAGCTGGACGGGGACGTAAACGGC |
| | | | CACAAGTTCAGCGTGTCCGGCGAGGGCGAGGGC |
| | | | GATGCCACCTACGGCAAGCTGACCCTGAAGTTC |
| | | | ATCTGCACCACCGGCAAGCTGCCCGTGCCCTGG |
| | | | CCCACCCTCGTGACCACCCTGACCTACGGCGTG |
| | | | CAGTGCTTCAGCCGCTACCCCGACCACATGAAG |
| | | | CAGCACGACTTCTTCAAGTCCGCCATGCCCGAA |
| | | | GGCTACGTCCAGGAGCGCACCATCTTCTTCAAG |
| | | | GACGACGGCAACTACAAGACCCGCGCCGAGGTG |
| | | | AAGTTCGAGGGCGACACCCTGGTGAACCGCATC |
| | | | GAGCTGAAGGGCATCGACTTCAAGGAGGACGGC |
| | | | AACATCCTGGGGCACAAGCTGGAGTACAACTAC |
| | | | AACAGCCACAACGTCTATATCATGGCCGACAAG |
| | | | CAGAAGAACGGCATCAAGGTGAACTTCAAGATC |
| | | | CGCCACAACATCGAGGACGGCAGCGTGCAGCTC |
| | | | GCCGACCACTACCAGCAGAACACCCCCATCGGC |
| | | | GACGGCCCCGTGCTGCTGCCCGACAACCACTAC |
| | | | CTGAGCACCCAGTCCGCCCTGAGCAAAGACCCC |
| | | | AACGAGAAGCGCGATCACATGGTCCTGCTGGAG |
| | | | TTCGTGACCGCCGCCGGGATCACTCTCGGCATG |
| | | | GACGAGCTGTACAAGTAACTCGAGTCTAGAGGG |
| | | | CCCGTTTAAACCCGCTGATCAGCCTCGACTGTG |
| | | | CCTTCTAGTTGCCAGCCATCTGTTGTTTGCCCC |
| | | | TCCCCCGTGCCTTCCTTGACCCTGGAAGGTGCC |
| | | | ACTCCCACTGTCCTTTCCTAATAAAATGAGGAA |
| | | | ATTGCATCGCATTGTCTGAGTAGGTGTCATTCT |
| | | | ATTCTGGGGGGTGGGGTGGGGCAGGACAGCAAG |
| | | | GGGGAGGATTGGGAAGACAATAGCAGGCATGCT |
| | | | GGGGATGCGGTGGGCTCTATGGCTTCTGAGGCG |
| | | | GAAAGAACCAGCTGGGGCTCTAGGGGGTATCCC |
| | | | CACGCGCCCTGTAGCGGCGCATTAAGCGCGGCG |
| | | | GGTGTGGTGGTTACGCGCAGCGTGACCGCTACA |
| | | | CTTGCCAGCGCCCTAGCGCCCGCTCCTTTCGCT |
| | | | TTCTTCCCTTCCTTTCTCGCCACGTTCGCCGGC |
| | | | TTTCCCCGTCAAGCTCTAAATCGGGGGTCCCTT |
| | | | TAGGGTTCCGATTTAGTGCTTTACGGCACCTCG |
| | | | ACCCCAAAAAACTTGATTAGGGTGATGGTTCAC |
| | | | GTACCTAGAAGTTCCTATTCCGAAGTTCCTATT |
| | | | CTCTAGAAAGTATAGGAACTTCCTTGGCCAAAA |
| | | | AGCCTGAACTCACCGCGACGTCTGTCGAGAAGT |
| | | | TTCTGATCGAAAAGTTCGACAGCGTCTCCGACC |
| | | | TGATGCAGCTCTCGGAGGGCGAAGAATCTCGTG |
| | | | CTTTCAGCTTCGATGTAGGAGGGCGTGGATATG |
| | | | TCCTGCGGGTAAATAGCTGCGCCGATGGTTTCT |
| | | | ACAAAGATCGTTATGTTTATCGGCACTTTGCAT |
| | | | CGGCCGCGCTCCCGATTCCGGAAGTGCTTGACA |
| | | | TTGGGGAATTCAGCGAGAGCCTGACCTATTGCA |
| | | | TCTCCCGCCGTGCACAGGGTGTCACGTTGCAAG |
| | | | ACCTGCCTGAAACCGAACTGCCCGCTGTTCTGC |
| | | | AGCCGGTCGCGGAGGCCATGGATGCGATCGCTG |
| | | | CGGCCGATCTTAGCCAGACGAGCGGGTTCGGCC |
| | | | CATTCGGACCGCAAGGAATCGGTCAATACACTA |
| | | | CATGGCGTGATTTCATATGCGCGATTGCTGATC |
| | | | CCCATGTGTATCACTGGCAAACTGTGATGGACG |
| | | | ACACCGTCAGTGCGTCCGTCGCGCAGGCTCTCG |
| | | | ATGAGCTGATGCTTTGGGCCGAGGACTGCCCCG |
| | | | AAGTCCGGCACCTCGTGCACGCGGATTTCGGCT |
| | | | CCAACAATGTCCTGACGGACAATGGCCGCATAA |
| | | | CAGCGGTCATTGACTGGAGCGAGGCGATGTTCG |
| | | | GGGATTCCCAATACGAGGTCGCCAACATCTTCT |
| | | | TCTGGAGGCCGTGGTTGGCTTGTATGGAGCAGC |
| | | | AGACGCGCTACTTCGAGCGGAGGCATCCGGAGC |
| | | | TTGCAGGATCGCCGCGGCTCCGGGCGTATATGC |
| | | | TCCGCATTGGTCTTGACCAACTCTATCAGAGCT |
| | | | TGGTTGACGGCAATTTCGATGATGCAGCTTGGG |
| | | | CGCAGGGTCGATGCGACGCAATCGTCCGATCCG |
| | | | GAGCCGGGACTGTCGGGCGTACACAAATCGCCC |
| | | | GCAGAAGCGCGGCCGTCTGGACCGATGGCTGTG |
| | | | TAGAAGTACTCGCCGATAGTGGAAACCGACGCC |
| | | | CCAGCACTCGTCCGAGGGCAAAGGAATAGCACG |
| | | | TACTACGAGATTTCGATTCCACCGCCGCCTTCT |
| | | | ATGAAAGGTTGGGCTTCGGAATCGTTTTCCGGG |
| | | | ACGCCGGCTGGATGATCCTCCAGCGCGGGGATC |
| | | | TCATGCTGGAGTTCTTCGCCCACCCCAACTTGT |
| | | | TTATTGCAGCTTATAATGGTTACAAATAAAGCA |
| | | | ATAGCATCACAAATTTCACAAATAAAGCATTTT |
| | | | TTTCACTGCATTCTAGTTGTGGTTTGTCCAAAC |
| | | | TCATCAATGTATCTTATCATGTCTGTATACCGT |
| | | | CGACCTCTAGCTAGAGCTTGGCGTAATCATGGT |
| | | | CATAGCTGTTTCCTGTGTGAAATTGTTATCCGC |
| | | | TCACAATTCCACACAACATACGAGCCGGAAGCA |
| | | | TAAAGTGTAAAGCCTGGGGTGCCTAATGAGTGA |
| | | | GCTAACTCACATTAATTGCGTTGCGCTCACTGC |
| | | | CCGCTTTCCAGTCGGGAAACCTGTCGTGCCAGC |
| | | | TGCATTAATGAATCGGCCAACGCGCGGGGAGAG |
| | | | GCGGTTTGCGTATTGGGCGCTCTTCCGCTTCCT |
| | | | CGCTCACTGACTCGCTGCGCTCGGTCGTTCGGC |
| | | | TGCGGCGAGCGGTATCAGCTCACTCAAAGGCGG |
| | | | TAATACGGTTATCCACAGAATCAGGGGATAACG |
| | | | CAGGAAAGAACATGTGAGCAAAAGGCCAGCAAA |
| | | | AGGCCAGGAACCGTAAAAAGGCCGCGTTGCTGG |
| | | | CGTTTTTCCATAGGCTCCGCCCCCCTGACGAGC |
| | | | ATCACAAAAATCGACGCTCAAGTCAGAGGTGGC |
| | | | GAAACCCGACAGGACTATAAAGATACCAGGCGT |
| | | | TTCCCCCTGGAAGCTCCCTCGTGCGCTCTCCTG |
| | | | TTCCGACCCTGCCGCTTACCGGATACCTGTCCG |
| | | | CCTTTCTCCCTTCGGGAAGCGTGGCGCTTTCTC |
| | | | ATAGCTCACGCTGTAGGTATCTCAGTTCGGTGT |
| | | | AGGTCGTTCGCTCCAAGCTGGGCTGTGTGCACG |
| | | | AACCCCCCGTTCAGCCCGACCGCTGCGCCTTAT |
| | | | CCGGTAACTATCGTCTTGAGTCCAACCCGGTAA |
| | | | GACACGACTTATCGCCACTGGCAGCAGCCACTG |
| | | | GTAACAGGATTAGCAGAGCGAGGTATGTAGGCG |
| | | | GTGCTACAGAGTTCTTGAAGTGGTGGCCTAACT |
| | | | ACGGCTACACTAGAAGGACAGTATTTGGTATCT |
| | | | GCGCTCTGCTGAAGCCAGTTACCTTCGGAAAAA |
| | | | GAGTTGGTAGCTCTTGATCCGGCAAACAAACCA |
| | | | CCGCTGGTAGCGGTGGTTTTTTTGTTTGCAAGC |
| | | | AGCAGATTACGCGCAGAAAAAAAGGATCTCAAG |
| | | | AAGATCCTTTGATCTTTTCTACGGGGTCTGACG |
| | | | CTCAGTGGAACGAAAACTCACGTTAAGGGATTT |
| | | | TGGTCATGAGATTATCAAAAAGGATCTTCACCT |
| | | | AGATCCTTTTAAATTAAAAATGAAGTTTTAAAT |
| | | | CAATCTAAAGTATATATGAGTAAACTTGGTCTG |
| | | | ACAGTTACCAATGCTTAATCAGTGAGGCACCTA |
| | | | TCTCAGCGATCTGTCTATTTCGTTCATCCATAG |
| | | | TTGCCTGACTCCCCGTCGTGTAGATAACTACGA |
| | | | TACGGGAGGGCTTACCATCTGGCCCCAGTGCTG |
| | | | CAATGATACCGCGAGACCCACGCTCACCGGCTC |
| | | | CAGATTTATCAGCAATAAACCAGCCAGCCGGAA |
| | | | GGGCCGAGCGCAGAAGTGGTCCTGCAACTTTAT |
| | | | CCGCCTCCATCCAGTCTATTAATTGTTGCCGGG |
| | | | AAGCTAGAGTAAGTAGTTCGCCAGTTAATAGTT |
| | | | TGCGCAACGTTGTTGCCATTGCTACAGGCATCG |
| | | | TGGTGTCACGCTCGTCGTTTGGTATGGCTTCAT |
| | | | TCAGCTCCGGTTCCCAACGATCAAGGCGAGTTA |
| | | | CATGATCCCCCATGTTGTGCAAAAAAGCGGTTA |
| | | | GCTCCTTCGGTCCTCCGATCGTTGTCAGAAGTA |
| | | | AGTTGGCCGCAGTGTTATCACTCATGGTTATGG |
| | | | CAGCACTGCATAATTCTCTTACTGTCATGCCAT |
| | | | CCGTAAGATGCTTTTCTGTGACTGGTGAGTACT |
| | | | CAACCAAGTCATTCTGAGAATAGTGTATGCGGC |
| | | | GACCGAGTTGCTCTTGCCCGGCGTCAATACGGG |
| | | | ATAATACCGCGCCACATAGCAGAACTTTAAAAG |
| | | | TGCTCATCATTGGAAAACGTTCTTCGGGGCGAA |
| | | | AACTCTCAAGGATCTTACCGCTGTTGAGATCCA |
| | | | GTTCGATGTAACCCACTCGTGCACCCAACTGAT |
| | | | CTTCAGCATCTTTTACTTTCACCAGCGTTTCTG |
| | | | GGTGAGCAAAAACAGGAAGGCAAAATGCCGCAA |
| | | | AAAAGGGAATAAGGGCGACACGGAAATGTTGAA |
| | | | TACTCATACTCTTCCTTTTTCAATATTATTGAA |
| | | | GCATTTATCAGGGTTATTGTCTCATGAGCGGAT |
| | | | ACATATTTGAATGTATTTAGAAAAATAAACAAA |
| | | | TAGGGGTTCCGCGCACATTTCCCCGAAAAGTGC |
| | | | CACCTGACGTC |
| SEQ ID | DNA | pb34 plasmid full | GACTCTTCGCGATGTACGGGCCAGATATACGCG |
| NO. 18 | | length DNA | TTGACATTGATTATTGACTAGTTATTAATAGTA |
| | | sequence | ATCAATTACGGGGTCATTAGTTCATAGCCCATA |
| | | | TATGGAGTTCCGCGTTACATAACTTACGGTAAA |
| | | | TGGCCCGCCTGGCTGACCGCCCAACGACCCCCG |
| | | | CCCATTGACGTCAATAATGACGTATGTTCCCAT |
| | | | AGTAACGCCAATAGGGACTTTCCATTGACGTCA |
| | | | ATGGGTGGACTATTTACGGTAAACTGCCCACTT |
| | | | GGCAGTACATCAAGTGTATCATATGCCAAGTAC |
| | | | GCCCCCTATTGACGTCAATGACGGTAAATGGCC |
| | | | CGCCTGGCATTATGCCCAGTACATGACCTTATG |
| | | | GGACTTTCCTACTTGGCAGTACATCTACGTATT |
| | | | AGTCATCGCTATTACCATGGTGATGCGGTTTTG |
| | | | GCAGTACATCAATGGGCGTGGATAGCGGTTTGA |
| | | | CTCACGGGGATTTCCAAGTCTCCACCCCATTGA |
| | | | CGTCAATGGGAGTTTGTTTTGGCACCAAAATCA |
| | | | ACGGGACTTTCCAAAATGTCGTAACAACTCCGC |
| | | | CCCATTGACGCAAATGGGCGGTAGGCGTGTACG |
| | | | GTGGGAGGTCTATATAAGCAGAGCTCTCTGGCT |
| | | | AACTAGAGAACCCACTGCTTACTGGCTTATCGA |
| | | | AATTAATACGACTCACTATAGGGAGACCCAAGC |
| | | | TGGCTAGCGTTTAAACTTAAGCTTATGGACTAC |
| | | | AAAGACCATGACGGTGATTATAAAGATCATGAC |
| | | | ATCGATTACAAGGATGACGATGACAAGATGGCC |
| | | | CCCAAGAAGAAGAGGAAGGTGGGCATTCACGGG |
| | | | GTACCGGCGGCGATGGCCGAGCGGCCCTTCCAG |
| | | | TGCAGGATCTGTATGCGCAACTTTTCTCGTTCT |
| | | | TCTGCTCTTACTCGTCACATCAGAACCCATACA |
| | | | GGCGAAAAGCCTTTCGCCTGCGACATTTGTGGG |
| | | | AGAAAATTTGCTCGTTCTGATACTCTTACTCGT |
| | | | CATACCAAGATCCACACCGGCTCTCAGAAACCA |
| | | | TTCCAGTGCCGCATTTGTATGCGGAATTTTTCC |
| | | | GATCGTTCTAATCTTACTCGTCATATCCGCACT |
| | | | CACACCGGAGAGAAGCCCTTTGCTTGCGACATT |
| | | | TGTGGCAGGAAATTTGCTCGTTCTGATAATCTT |
| | | | ACTCGTCACACTAAGATCCATACTGGGTCACAG |
| | | | AAACCTTTCCAGTGCCGGATTTGTATGAGAAAC |
| | | | TTTAGCCGTTCTGATCATCTTACTCGTCACATC |
| | | | AGAACACATACTGGGCTGAGAGGATCCAATTCT |
| | | | GGTGATCCTCGGAGACACAGTCTGGGCGGTTCT |
| | | | CGTAAACCCGATCTGATTGCCTATAAAAACTTT |
| | | | GATCTGCTGGTCATTGTTCTTAAGCCTTGAGCG |
| | | | GCCGCTCGAGTCTAGAGGGCCCGTTTAAACCCG |
| | | | CTGATCAGCCTCGACTGTGCCTTCTAGTTGCCA |
| | | | GCCATCTGTTGTTTGCCCCTCCCCCGTGCCTTC |
| | | | CTTGACCCTGGAAGGTGCCACTCCCACTGTCCT |
| | | | TTCCTAATAAAATGAGGAAATTGCATCGCATTG |
| | | | TCTGAGTAGGTGTCATTCTATTCTGGGGGGTGG |
| | | | GGTGGGGCAGGACAGCAAGGGGGAGGATTGGGA |
| | | | AGACAATAGCAGGCATGCTGGGGATGCGGTGGG |
| | | | CTCTATGGCTTCTACTGGGCGGTTTTATGGACA |
| | | | GCAAGCGAACCGGAATTGCCAGCTGGGGCGCCC |
| | | | TCTGGTAAGGTTGGGAAGCCCTGCAAAGTAAAC |
| | | | TGGATGGCTTTCTCGCCGCCAAGGATCTGATGG |
| | | | CGCAGGGGATCAAGCTCTGATCAAGAGACAGGA |
| | | | TGAGGATCGTTTCGCATGATTGAACAAGATGGA |
| | | | TTGCACGCAGGTTCTCCGGCCGCTTGGGTGGAG |
| | | | AGGCTATTCGGCTATGACTGGGCACAACAGACA |
| | | | ATCGGCTGCTCTGATGCCGCCGTGTTCCGGCTG |
| | | | TCAGCGCAGGGGCGCCCGGTTCTTTTTGTCAAG |
| | | | ACCGACCTGTCCGGTGCCCTGAATGAACTGCAA |
| | | | GACGAGGCAGCGCGGCTATCGTGGCTGGCCACG |
| | | | ACGGGCGTTCCTTGCGCAGCTGTGCTCGACGTT |
| | | | GTCACTGAAGCGGGAAGGGACTGGCTGCTATTG |
| | | | GGCGAAGTGCCGGGGCAGGATCTCCTGTCATCT |
| | | | CACCTTGCTCCTGCCGAGAAAGTATCCATCATG |
| | | | GCTGATGCAATGCGGCGGCTGCATACGCTTGAT |
| | | | CCGGCTACCTGCCCATTCGACCACCAAGCGAAA |
| | | | CATCGCATCGAGCGAGCACGTACTCGGATGGAA |
| | | | GCCGGTCTTGTCGATCAGGATGATCTGGACGAA |
| | | | GAGCATCAGGGGCTCGCGCCAGCCGAACTGTTC |
| | | | GCCAGGCTCAAGGCGAGCATGCCCGACGGCGAG |
| | | | GATCTCGTCGTGACCCATGGCGATGCCTGCTTG |
| | | | CCGAATATCATGGTGGAAAATGGCCGCTTTTCT |
| | | | GGATTCATCGACTGTGGCCGGCTGGGTGTGGCG |
| | | | GACCGCTATCAGGACATAGCGTTGGCTACCCGT |
| | | | GATATTGCTGAAGAGCTTGGCGGCGAATGGGCT |
| | | | GACCGCTTCCTCGTGCTTTACGGTATCGCCGCT |
| | | | CCCGATTCGCAGCGCATCGCCTTCTATCGCCTT |
| | | | CTTGACGAGTTCTTCTGAATTATTAACGCTTAC |
| | | | AATTTCCTGATGCGGTATTTTCTCCTTACGCAT |
| | | | CTGTGCGGTATTTCACACCGCATACAGGTGGCA |
| | | | CTTTTCGGGGAAATGTGCGCGGAACCCCTATTT |
| | | | GTTTATTTTTCTAAATACATTCAAATATGTATC |
| | | | CGCTCATGAGACAATAACCCTGATAAATGCTTC |
| | | | AATAATAGCACGTGCTAAAACTTCATTTTTAAT |
| | | | TTAAAAGGATCTAGGTGAAGATCCTTTTTGATA |
| | | | ATCTCATGACCAAAATCCCTTAACGTGAGTTTT |
| | | | CGTTCCACTGAGCGTCAGACCCCGTAGAAAAGA |
| | | | TCAAAGGATCTTCTTGAGATCCTTTTTTTCTGC |
| | | | GCGTAATCTGCTGCTTGCAAACAAAAAAACCAC |
| | | | CGCTACCAGCGGTGGTTTGTTTGCCGGATCAAG |
| | | | AGCTACCAACTCTTTTTCCGAAGGTAACTGGCT |
| | | | TCAGCAGAGCGCAGATACCAAATACTGTCCTTC |
| | | | TAGTGTAGCCGTAGTTAGGCCACCACTTCAAGA |
| | | | ACTCTGTAGCACCGCCTACATACCTCGCTCTGC |
| | | | TAATCCTGTTACCAGTGGCTGCTGCCAGTGGCG |
| | | | ATAAGTCGTGTCTTACCGGGTTGGACTCAAGAC |
| | | | GATAGTTACCGGATAAGGCGCAGCGGTCGGGCT |
| | | | GAACGGGGGGTTCGTGCACACAGCCCAGCTTGG |
| | | | AGCGAACGACCTACACCGAACTGAGATACCTAC |
| | | | AGCGTGAGCTATGAGAAAGCGCCACGCTTCCCG |
| | | | AAGGGAGAAAGGCGGACAGGTATCCGGTAAGCG |
| | | | GCAGGGTCGGAACAGGAGAGCGCACGAGGGAGC |
| | | | TTCCAGGGGGAAACGCCTGGTATCTTTATAGTC |
| | | | CTGTCGGGTTTCGCCACCTCTGACTTGAGCGTC |
| | | | GATTTTTGTGATGCTCGTCAGGGGGGCGGAGCC |
| | | | TATGGAAAAACGCCAGCAACGCGGCCTTTTTAC |
| | | | GGTTCCTGGGCTTTTGCTGGCCTTTTGCTCACA |
| | | | TGTTCTT |
| SEQ ID | Amino | R domain of | NSGDPRRHSLGGSRKPDLIAYKNFDLLVIVLKP |
| NO. 19 | Acid | EGFPDP2 DLR, | |
| | | encoded in pb34 | |
| SEQ ID | DNA | R domain coding | AATTCTGGTGATCCTCGGAGACACAGTCTGGGC |
| NO. 20 | | sequence of plasmid | GGTTCTCGTAAACCCGATCTGATTGCCTATAAA |
| | | pb34 | AACTTTGATCTGCTGGTCATTGTTCTTAAGCCT |
| | | | TGA |
| SEQ ID | DNA | pb6 plasmid full | GACTCTTCGCGATGTACGGGCCAGATATACGCG |
| NO. 21 | | length DNA | TTGACATTGATTATTGACTAGTTATTAATAGTA |
| | | sequence | ATCAATTACGGGGTCATTAGTTCATAGCCCATA |
| | | | TATGGAGTTCCGCGTTACATAACTTACGGTAAA |
| | | | TGGCCCGCCTGGCTGACCGCCCAACGACCCCCG |
| | | | CCCATTGACGTCAATAATGACGTATGTTCCCAT |
| | | | AGTAACGCCAATAGGGACTTTCCATTGACGTCA |
| | | | ATGGGTGGACTATTTACGGTAAACTGCCCACTT |
| | | | GGCAGTACATCAAGTGTATCATATGCCAAGTAC |
| | | | GCCCCCTATTGACGTCAATGACGGTAAATGGCC |
| | | | CGCCTGGCATTATGCCCAGTACATGACCTTATG |
| | | | GGACTTTCCTACTTGGCAGTACATCTACGTATT |
| | | | AGTCATCGCTATTACCATGGTGATGCGGTTTTG |
| | | | GCAGTACATCAATGGGCGTGGATAGCGGTTTGA |
| | | | CTCACGGGGATTTCCAAGTCTCCACCCCATTGA |
| | | | CGTCAATGGGAGTTTGTTTTGGCACCAAAATCA |
| | | | ACGGGACTTTCCAAAATGTCGTAACAACTCCGC |
| | | | CCCATTGACGCAAATGGGCGGTAGGCGTGTACG |
| | | | GTGGGAGGTCTATATAAGCAGAGCTCTCTGGCT |
| | | | AACTAGAGAACCCACTGCTTACTGGCTTATCGA |
| | | | AATTAATACGACTCACTATAGGGAGACCCAAGC |
| | | | TGGCTAGCGTTTAAACTTAAGCTTATGGCGGCG |
| | | | ATGGCCGAGCGGCCCTTCCAGTGCAGGATCTGT |
| | | | ATGCGCAACTTTTCTCGGTCCTCCGACCTGACC |
| | | | CGGCACATCAGAACCCATACAGGCGAAAAGCCT |
| | | | TTCGCCTGCGACATTTGTGGGAGAAAATTTGCT |
| | | | CGGTCCGACACCCTGACCCGGCATACCAAGATC |
| | | | CACACCGGCTCTCAGAAACCATTCCAGTGCCGC |
| | | | ATTTGTATGCGGAATTTTTCCCAGTCCGGCGAC |
| | | | CTGTCCGAGCATATCCGCACTCACACCGGAGAG |
| | | | AAGCCCTTTGCTTGCGACATTTGTGGCAGGAAA |
| | | | TTTGCTACCTCCGGCCACCTGACCACCCACACT |
| | | | AAGATCCATACTGGGTCACAGAAACCTTTCCAG |
| | | | TGCCGGATTTGTATGAGAAACTTTAGCGACTCC |
| | | | TCCCACCTGACCACCCACATCAGAACCCATACA |
| | | | GGCGAAAAGCCTTTCGCCTGCGACATTTGTGGG |
| | | | AGAAAATTTGCTCGGTCCTCCCACCTGACCACC |
| | | | CATACCAAGATCCACACCGGCTCTCAGAAACCA |
| | | | TTCCAGTGCCGCATTTGTATGCGGAATTTTTCC |
| | | | GACCGGTCCGACCTGACCCGGCATATCCGCACT |
| | | | CACACCGGAGAGAAGCCCTTTGCTTGCGACATT |
| | | | TGTGGCAGGAAATTTGCTGACCGGTCCGACCTG |
| | | | ACCCGGCACACTAAGATCCATACTGGGTCACAG |
| | | | AAACCTTTCCAGTGCCGGATTTGTATGAGAAAC |
| | | | TTTAGCCGGTCCGACACCCTGACCCGGCACATC |
| | | | AGAACACATACTGGGCTGAGAGGATCCAATTCT |
| | | | GGTGATCCTCGGAGACACAGTCTGGGCGGTTCT |
| | | | CGTAAACCCGATCTGATTGCCTATAAAAACTTT |
| | | | GATCTGCTGGTCATTGTTCTTAAGCCTTGAGCG |
| | | | GCCGCTCGAGTCTAGAGGGCCCGTTTAAACCCG |
| | | | CTGATCAGCCTCGACTGTGCCTTCTAGTTGCCA |
| | | | GCCATCTGTTGTTTGCCCCTCCCCCGTGCCTTC |
| | | | CTTGACCCTGGAAGGTGCCACTCCCACTGTCCT |
| | | | TTCCTAATAAAATGAGGAAATTGCATCGCATTG |
| | | | TCTGAGTAGGTGTCATTCTATTCTGGGGGGTGG |
| | | | GGTGGGGCAGGACAGCAAGGGGGAGGATTGGGA |
| | | | AGACAATAGCAGGCATGCTGGGGATGCGGTGGG |
| | | | CTCTATGGCTTCTACTGGGCGGTTTTATGGACA |
| | | | GCAAGCGAACCGGAATTGCCAGCTGGGGCGCCC |
| | | | TCTGGTAAGGTTGGGAAGCCCTGCAAAGTAAAC |
| | | | TGGATGGCTTTCTCGCCGCCAAGGATCTGATGG |
| | | | CGCAGGGGATCAAGCTCTGATCAAGAGACAGGA |
| | | | TGAGGATCGTTTCGCATGATTGAACAAGATGGA |
| | | | TTGCACGCAGGTTCTCCGGCCGCTTGGGTGGAG |
| | | | AGGCTATTCGGCTATGACTGGGCACAACAGACA |
| | | | ATCGGCTGCTCTGATGCCGCCGTGTTCCGGCTG |
| | | | TCAGCGCAGGGGCGCCCGGTTCTTTTTGTCAAG |
| | | | ACCGACCTGTCCGGTGCCCTGAATGAACTGCAA |
| | | | GACGAGGCAGCGCGGCTATCGTGGCTGGCCACG |
| | | | ACGGGCGTTCCTTGCGCAGCTGTGCTCGACGTT |
| | | | GTCACTGAAGCGGGAAGGGACTGGCTGCTATTG |
| | | | GGCGAAGTGCCGGGGCAGGATCTCCTGTCATCT |
| | | | CACCTTGCTCCTGCCGAGAAAGTATCCATCATG |
| | | | GCTGATGCAATGCGGCGGCTGCATACGCTTGAT |
| | | | CCGGCTACCTGCCCATTCGACCACCAAGCGAAA |
| | | | CATCGCATCGAGCGAGCACGTACTCGGATGGAA |
| | | | GCCGGTCTTGTCGATCAGGATGATCTGGACGAA |
| | | | GAGCATCAGGGGCTCGCGCCAGCCGAACTGTTC |
| | | | GCCAGGCTCAAGGCGAGCATGCCCGACGGCGAG |
| | | | GATCTCGTCGTGACCCATGGCGATGCCTGCTTG |
| | | | CCGAATATCATGGTGGAAAATGGCCGCTTTTCT |
| | | | GGATTCATCGACTGTGGCCGGCTGGGTGTGGCG |
| | | | GACCGCTATCAGGACATAGCGTTGGCTACCCGT |
| | | | GATATTGCTGAAGAGCTTGGCGGCGAATGGGCT |
| | | | GACCGCTTCCTCGTGCTTTACGGTATCGCCGCT |
| | | | CCCGATTCGCAGCGCATCGCCTTCTATCGCCTT |
| | | | CTTGACGAGTTCTTCTGAATTATTAACGCTTAC |
| | | | AATTTCCTGATGCGGTATTTTCTCCTTACGCAT |
| | | | CTGTGCGGTATTTCACACCGCATACAGGTGGCA |
| | | | CTTTTCGGGGAAATGTGCGCGGAACCCCTATTT |
| | | | GTTTATTTTTCTAAATACATTCAAATATGTATC |
| | | | CGCTCATGAGACAATAACCCTGATAAATGCTTC |
| | | | AATAATAGCACGTGCTAAAACTTCATTTTTAAT |
| | | | TTAAAAGGATCTAGGTGAAGATCCTTTTTGATA |
| | | | ATCTCATGACCAAAATCCCTTAACGTGAGTTTT |
| | | | CGTTCCACTGAGCGTCAGACCCCGTAGAAAAGA |
| | | | TCAAAGGATCTTCTTGAGATCCTTTTTTTCTGC |
| | | | GCGTAATCTGCTGCTTGCAAACAAAAAAACCAC |
| | | | CGCTACCAGCGGTGGTTTGTTTGCCGGATCAAG |
| | | | AGCTACCAACTCTTTTTCCGAAGGTAACTGGCT |
| | | | TCAGCAGAGCGCAGATACCAAATACTGTCCTTC |
| | | | TAGTGTAGCCGTAGTTAGGCCACCACTTCAAGA |
| | | | ACTCTGTAGCACCGCCTACATACCTCGCTCTGC |
| | | | TAATCCTGTTACCAGTGGCTGCTGCCAGTGGCG |
| | | | ATAAGTCGTGTCTTACCGGGTTGGACTCAAGAC |
| | | | GATAGTTACCGGATAAGGCGCAGCGGTCGGGCT |
| | | | GAACGGGGGGTTCGTGCACACAGCCCAGCTTGG |
| | | | AGCGAACGACCTACACCGAACTGAGATACCTAC |
| | | | AGCGTGAGCTATGAGAAAGCGCCACGCTTCCCG |
| | | | AAGGGAGAAAGGCGGACAGGTATCCGGTAAGCG |
| | | | GCAGGGTCGGAACAGGAGAGCGCACGAGGGAGC |
| | | | TTCCAGGGGGAAACGCCTGGTATCTTTATAGTC |
| | | | CTGTCGGGTTTCGCCACCTCTGACTTGAGCGTC |
| | | | GATTTTTGTGATGCTCGTCAGGGGGGCGGAGCC |
| | | | TATGGAAAAACGCCAGCAACGCGGCCTTTTTAC |
| | | | GGTTCCTGGGCTTTTGCTGGCCTTTTGCTCACA |
| | | | TGTTCTT |
| SEQ ID | DNA | POP33, donor | CCCCGGTGGCGGAGGAGACGCGGGCACGGCTGT |
| NO. 22 | | template | CCAAGGAGCTGCAGGCGGCGCAGGCCCGGCTGG |
| | | | GCGCGGACATGGAGGACGTGCGCGGCCGCCTGG |
| | | | TGCAGTACCGCGGCGAGGTGCAGGCCATGC |
| SEQ ID | DNA | POP7, donor | CCCCGGTGGCGGAGGAGACGCGGGCACGGCTGT |
| NO. 23 | | template | CCAAGGAGCTGCAGGCGGCGCAGGCCCGGCTGG |
| | | | GCGCGGACATGGAGGACGTGCGCGGCCGCCTGG |
| | | | TGCAGTACCGCGGCGAGGTGCAGGCCATGCTCG |
| | | | GCCAGAGCACCGAGGAGC |
| SEQ ID | DNA | Pop46-511-Alu- | CTGCAGGCGGCGCAGGC |
| NO. 24 | | apoE-f forward | |
| | | primer | |
| SEQ ID | DNA | Pop47-512-Alu- | CTCCTCGGTGCTCTGGCCGA |
| NO. 25 | | apoE-r reverse | |
| | | primer | |
| SEQ ID | DNA | POP58 512′ F+fwd | ACACTCTTTCCCTACACGACGCTCTTCCGATCT |
| NO. 26 | | sequencing read tag | TCGGCCAGAGCACCGAGGAG |
| | | primer | |
| SEQ ID | DNA | POP59 512x R+rvs | GACTGGAGTTCAGACGTGTGCTCTTCCGATCTG |
| NO. 27 | DNA | sequencing read tag | CATGGCCTGCACCTCGC |
| SEQ ID | | primer | GACTCTTCGCGATGTACGGGCCAGATATACGCG |
| NO. 28 | | pb41 plasmid full | TTGACATTGATTATTGACTAGTTATTAATAGTA |
| | | length DNA | ATCAATTACGGGGTCATTAGTTCATAGCCCATA |
| | | sequence | TATGGAGTTCCGCGTTACATAACTTACGGTAAA |
| | | | TGGCCCGCCTGGCTGACCGCCCAACGACCCCCG |
| | | | CCCATTGACGTCAATAATGACGTATGTTCCCAT |
| | | | AGTAACGCCAATAGGGACTTTCCATTGACGTCA |
| | | | ATGGGTGGACTATTTACGGTAAACTGCCCACTT |
| | | | GGCAGTACATCAAGTGTATCATATGCCAAGTAC |
| | | | GCCCCCTATTGACGTCAATGACGGTAAATGGCC |
| | | | CGCCTGGCATTATGCCCAGTACATGACCTTATG |
| | | | GGACTTTCCTACTTGGCAGTACATCTACGTATT |
| | | | AGTCATCGCTATTACCATGGTGATGCGGTTTTG |
| | | | GCAGTACATCAATGGGCGTGGATAGCGGTTTGA |
| | | | CTCACGGGGATTTCCAAGTCTCCACCCCATTGA |
| | | | CGTCAATGGGAGTTTGTTTTGGCACCAAAATCA |
| | | | ACGGGACTTTCCAAAATGTCGTAACAACTCCGC |
| | | | CCCATTGACGCAAATGGGCGGTAGGCGTGTACG |
| | | | GTGGGAGGTCTATATAAGCAGAGCTCTCTGGCT |
| | | | AACTAGAGAACCCACTGCTTACTGGCTTATCGA |
| | | | AATTAATACGACTCACTATAGGGAGACCCAAGC |
| | | | TGGCTAGCGTTTAAACTTAAGCTTATGGCGGCG |
| | | | ATGGCCGAGCGGCCCTTCCAGTGCAGGATCTGT |
| | | | ATGCGCAACTTTTCTGACCGGTCCCACCTGACC |
| | | | CGGCACATCAGAACCCATACAGGCGAAAAGCCT |
| | | | TTCGCCTGCGACATTTGTGGGAGAAAATTTGCT |
| | | | CGGTCCGACAACCTGACCCGGCATACCAAGATC |
| | | | CACACCGGCTCTCAGAAACCATTCCAGTGCCGC |
| | | | ATTTGTATGCGGAATTTTTCCGACTCCTCCCAC |
| | | | CTGTCCGAGCATATCCGCACTCACACCGGAGAG |
| | | | AAGCCCTTTGCTTGCGACATTTGTGGCAGGAAA |
| | | | TTTGCTGACCGGTCCGACCTGACCCGGCACACT |
| | | | AAGATCCATACTGGGTCACAGAAACCTTTCCAG |
| | | | TGCCGGATTTGTATGAGAAACTTTAGCCGGTCC |
| | | | GACCACCTGACCCGGCACATCAGAACCCATACA |
| | | | GGCGAAAAGCCTTTCGCCTGCGACATTTGTGGG |
| | | | AGAAAATTTGCTGACCGGTCCGACCTGACCCGG |
| | | | CATACCAAGATCCACACCGGCTCTCAGAAACCA |
| | | | TTCCAGTGCCGCATTTGTATGCGGAATTTTTCC |
| | | | CGGTCCGACAACCTGTCCGAGCATATCCGCACT |
| | | | CACACCGGAGAGAAGCCCTTTGCTTGCGACATT |
| | | | TGTGGCAGGAAATTTGCTGAGTCCTCCAACCTG |
| | | | ACCACCCATACCAAGATCCACACCGGCTCTCAG |
| | | | AAACCATTCCAGTGCCGCATTTGTATGCGGAAT |
| | | | TTTTCCCGGTCCTCCTCCCTGACCCGGCATATC |
| | | | CGCACTCACACCGGAGAGAAGCCCTTTGCTTGC |
| | | | GACATTTGTGGCAGGAAATTTGCTCAGTCCTCC |
| | | | GACCTGACCCGGCACACTAAGATCCATACTGGG |
| | | | TCACAGAAACCTTTCCAGTGCCGGATTTGTATG |
| | | | AGAAACTTTAGCCGGTCCGACTCCCTGTCCGAG |
| | | | CACATCAGAACACATACTGGGCTGAGAGGATCC |
| | | | AATTCTGGTGATCCTCGGAGACACAGTCTGGGC |
| | | | GGTTCTCGTAAACCCGATCTGATTGCCTATAAA |
| | | | AACTTTGATCTGCTGGTCATTGTTCTTAAGCCT |
| | | | TGAGCGGCCGCTCGAGTCTAGAGGGCCCGTTTA |
| | | | AACCCGCTGATCAGCCTCGACTGTGCCTTCTAG |
| | | | TTGCCAGCCATCTGTTGTTTGCCCCTCCCCCGT |
| | | | GCCTTCCTTGACCCTGGAAGGTGCCACTCCCAC |
| | | | TGTCCTTTCCTAATAAAATGAGGAAATTGCATC |
| | | | GCATTGTCTGAGTAGGTGTCATTCTATTCTGGG |
| | | | GGGTGGGGTGGGGCAGGACAGCAAGGGGGAGGA |
| | | | TTGGGAAGACAATAGCAGGCATGCTGGGGATGC |
| | | | GGTGGGCTCTATGGCTTCTACTGGGCGGTTTTA |
| | | | TGGACAGCAAGCGAACCGGAATTGCCAGCTGGG |
| | | | GCGCCCTCTGGTAAGGTTGGGAAGCCCTGCAAA |
| | | | GTAAACTGGATGGCTTTCTCGCCGCCAAGGATC |
| | | | TGATGGCGCAGGGGATCAAGCTCTGATCAAGAG |
| | | | ACAGGATGAGGATCGTTTCGCATGATTGAACAA |
| | | | GATGGATTGCACGCAGGTTCTCCGGCCGCTTGG |
| | | | GTGGAGAGGCTATTCGGCTATGACTGGGCACAA |
| | | | CAGACAATCGGCTGCTCTGATGCCGCCGTGTTC |
| | | | CGGCTGTCAGCGCAGGGGCGCCCGGTTCTTTTT |
| | | | GTCAAGACCGACCTGTCCGGTGCCCTGAATGAA |
| | | | CTGCAAGACGAGGCAGCGCGGCTATCGTGGCTG |
| | | | GCCACGACGGGCGTTCCTTGCGCAGCTGTGCTC |
| | | | GACGTTGTCACTGAAGCGGGAAGGGACTGGCTG |
| | | | CTATTGGGCGAAGTGCCGGGGCAGGATCTCCTG |
| | | | TCATCTCACCTTGCTCCTGCCGAGAAAGTATCC |
| | | | ATCATGGCTGATGCAATGCGGCGGCTGCATACG |
| | | | CTTGATCCGGCTACCTGCCCATTCGACCACCAA |
| | | | GCGAAACATCGCATCGAGCGAGCACGTACTCGG |
| | | | ATGGAAGCCGGTCTTGTCGATCAGGATGATCTG |
| | | | GACGAAGAGCATCAGGGGCTCGCGCCAGCCGAA |
| | | | CTGTTCGCCAGGCTCAAGGCGAGCATGCCCGAC |
| | | | GGCGAGGATCTCGTCGTGACCCATGGCGATGCC |
| | | | TGCTTGCCGAATATCATGGTGGAAAATGGCCGC |
| | | | TTTTCTGGATTCATCGACTGTGGCCGGCTGGGT |
| | | | GTGGCGGACCGCTATCAGGACATAGCGTTGGCT |
| | | | ACCCGTGATATTGCTGAAGAGCTTGGCGGCGAA |
| | | | TGGGCTGACCGCTTCCTCGTGCTTTACGGTATC |
| | | | GCCGCTCCCGATTCGCAGCGCATCGCCTTCTAT |
| | | | CGCCTTCTTGACGAGTTCTTCTGAATTATTAAC |
| | | | GCTTACAATTTCCTGATGCGGTATTTTCTCCTT |
| | | | ACGCATCTGTGCGGTATTTCACACCGCATACAG |
| | | | GTGGCACTTTTCGGGGAAATGTGCGCGGAACCC |
| | | | CTATTTGTTTATTTTTCTAAATACATTCAAATA |
| | | | TGTATCCGCTCATGAGACAATAACCCTGATAAA |
| | | | TGCTTCAATAATAGCACGTGCTAAAACTTCATT |
| | | | TTTAATTTAAAAGGATCTAGGTGAAGATCCTTT |
| | | | TTGATAATCTCATGACCAAAATCCCTTAACGTG |
| | | | AGTTTTCGTTCCACTGAGCGTCAGACCCCGTAG |
| | | | AAAAGATCAAAGGATCTTCTTGAGATCCTTTTT |
| | | | TTCTGCGCGTAATCTGCTGCTTGCAAACAAAAA |
| | | | AACCACCGCTACCAGCGGTGGTTTGTTTGCCGG |
| | | | ATCAAGAGCTACCAACTCTTTTTCCGAAGGTAA |
| | | | CTGGCTTCAGCAGAGCGCAGATACCAAATACTG |
| | | | TCCTTCTAGTGTAGCCGTAGTTAGGCCACCACT |
| | | | TCAAGAACTCTGTAGCACCGCCTACATACCTCG |
| | | | CTCTGCTAATCCTGTTACCAGTGGCTGCTGCCA |
| | | | GTGGCGATAAGTCGTGTCTTACCGGGTTGGACT |
| | | | CAAGACGATAGTTACCGGATAAGGCGCAGCGGT |
| | | | CGGGCTGAACGGGGGGTTCGTGCACACAGCCCA |
| | | | GCTTGGAGCGAACGACCTACACCGAACTGAGAT |
| | | | ACCTACAGCGTGAGCTATGAGAAAGCGCCACGC |
| | | | TTCCCGAAGGGAGAAAGGCGGACAGGTATCCGG |
| | | | TAAGCGGCAGGGTCGGAACAGGAGAGCGCACGA |
| | | | GGGAGCTTCCAGGGGGAAACGCCTGGTATCTTT |
| | | | ATAGTCCTGTCGGGTTTCGCCACCTCTGACTTG |
| | | | AGCGTCGATTTTTGTGATGCTCGTCAGGGGGGC |
| | | | GGAGCCTATGGAAAAACGCCAGCAACGCGGCCT |
| | | | TTTTACGGTTCCTGGGCTTTTGCTGGCCTTTTG |
| | | | CTCACATGTTCTT |
| SEQ ID | DNA | 514-ODN-ApoE- | GCGGGTGCGCCTCGCCTCCCACCTGCGCAAGCT |
| NO. 29 | | C158 f donor | GCGTAAGCGGCTCCTCCGCGATGCCGATGACCT |
| | | template | GCAGAAGtGCCTGGCAGTGTACCAGGCCGGGGC |
| | | | CCGCGAGGGCGCCGAGCGCGGCCTCAGCGCCAT |
| | | | CCGCGAGCGCCTGGGGCC |
| SEQ ID | DNA | 515-ODN-ApoE- | GGCCCCAGGCGCTCGCGGATGGCGCTGAGGCCG |
| NO. 30 | | C158 r donor | CGCTCGGCGCCCTCGCGGGCCCCGGCCTGGTAC |
| | | template | ACTGCCAGGCaCTTCTGCAGGTCATCGGCATCG |
| | | | CGGAGGAGCCGCTTACGCAGCTTGCGCAGGTGG |
| | | | GAGGCGAGGCGCACCCGC |
| SEQ ID | DNA | 520-ODN- | CCGGCTGGGCGCGGACATGGAGGACGTGCGCGG |
| NO. 31 | | R112C158 f donor | CCGCCTGGTGCAGTACCGCGGCGAGGTGCAGGC |
| | | template | CATGCTCGGCCAGAGCACCGAGGAGCTGCGGGT |
| | | | GCGCCTCGCCTCCCACCTGCGCAAGCTGCGTAA |
| | | | GCGGCTCCTCCGCGATGCCGATGACCTGCAGAA |
| | | | GTGCCTGGCAGTGTACCAGGCCGGGGCCCGCGA |
| | | | GG |
| SEQ ID | DNA | 521-ODN- | CCTCGCGGGCCCCGGCCTGGTACACTGCCAGGC |
| NO. 32 | | R112C158 r donor | ACTTCTGCAGGTCATCGGCATCGCGGAGGAGCC |
| | | template | GCTTACGCAGCTTGCGCAGGTGGGAGGCGAGGC |
| | | | GCACCCGCAGCTCCTCGGTGCTCTGGCCGAGCA |
| | | | TGGCCTGCACCTCGCCGCGGTACTGCACCAGGC |
| | | | GGCCGCGCACGTCCTCCATGTCCGCGCCCAGCC |
| | | | GG |
| SEQ ID | DNA | 482-ODN-Odn E2 F | CCCCGGTGGCGGAGGAGACGCGGGCACGGCTGT |
| NO. 33 | | donor template | CCAAGGAGCTGCAGGCGGCGCAGGCCCGGCTGG |
| | | | GCGCGGACATGGAGGACGTGTGCGGCCGCCTGG |
| | | | TGCAGTACCGCGGCGAGGTGCAGGCCATGCTCG |
| | | | GCCAGAGCACCGAGGAGC |
| SEQ ID | Amino | pb1 | MAAMAERPFQCRICMRNFSRSSDLTRHIRTHTG |
| NO. 34 | Acid | | EKPFACDICGRKFARSDTLTRHTKIHTGSQKPF |
| | | | QCRICMRNFSQSGDLSEHIRTHTGEKPFACDIC |
| | | | GRKFATSGHLTTHTKIHTGSQKPFQCRICMRNF |
| | | | SDSSHLTTHIRTHTGEKPFACDICGRKFARSSH |
| | | | LTTHTKIHTGSQKPFQCRICMRNFSDRSDLTRH |
| | | | IRTHTGEKPFACDICGRKFADRSDLTRHTKIHT |
| | | | GSQKPFQCRICMRNFSRSDTLTRHIRTHTGLRG |
| | | | SNSGDPRRHSLGGSRKPDLIAYKNFDLLVIQLK |
| | | | P* |
| SEQ ID | Amino | pb2 | MAAMAERPFQCRICMRNFSRSSDLTRHIRTHTG |
| NO. 35 | Acid | | EKPFACDICGRKFARSDTLTRHTKIHTGSQKPF |
| | | | QCRICMRNFSQSGDLSEHIRTHTGEKPFACDIC |
| | | | GRKFATSGHLTTHTKIHTGSQKPFQCRICMRNF |
| | | | SDSSHLTTHIRTHTGEKPFACDICGRKFARSSH |
| | | | LTTHTKIHTGSQKPFQCRICMRNFSDRSDLTRH |
| | | | IRTHTGEKPFACDICGRKFADRSDLTRHTKIHT |
| | | | GSQKPFQCRICMRNFSRSDTLTRHIRTHTGLRG |
| | | | SNSGDPRRHSLGGSRKPDLIAYKNFDLLVINLK |
| | | | Px |
| SEQ ID | Amino | pb3 | MAAMAERPFQCRICMRNFSRSSDLTRHIRTHTG |
| NO. 36 | Acid | | EKPFACDICGRKFARSDTLTRHTKIHTGSQKPF |
| | | | QCRICMRNFSQSGDLSEHIRTHTGEKPFACDIC |
| | | | GRKFATSGHLTTHTKIHTGSQKPFQCRICMRNF |
| | | | SDSSHLTTHIRTHTGEKPFACDICGRKFARSSH |
| | | | LTTHTKIHTGSQKPFQCRICMRNFSDRSDLTRH |
| | | | IRTHTGEKPFACDICGRKFADRSDLTRHTKIHT |
| | | | GSQKPFQCRICMRNFSRSDTLTRHIRTHTGLRG |
| | | | SNSGDPRRHSLGGSRKPDLIAYKNFDLLVISLK |
| | | | Px |
| SEQ ID | Amino | pb4 | MAAMAERPFQCRICMRNFSRSSDLTRHIRTHTG |
| NO. 37 | Acid | | EKPFACDICGRKFARSDTLTRHTKIHTGSQKPF |
| | | | QCRICMRNFSQSGDLSEHIRTHTGEKPFACDIC |
| | | | GRKFATSGHLTTHTKIHTGSQKPFQCRICMRNF |
| | | | SDSSHLTTHIRTHTGEKPFACDICGRKFARSSH |
| | | | LTTHTKIHTGSQKPFQCRICMRNFSDRSDLTRH |
| | | | IRTHTGEKPFACDICGRKFADRSDLTRHTKIHT |
| | | | GSQKPFQCRICMRNFSRSDTLTRHIRTHTGLRG |
| | | | SNSGDPRRHSLGGSRKPDLIAYKNFDLLVITLK |
| | | | P* |
| SEQ ID | Amino | pb5 | MAAMAERPFQCRICMRNFSRSSDLTRHIRTHTG |
| NO. 38 | Acid | | EKPFACDICGRKFARSDTLTRHTKIHTGSQKPF |
| | | | QCRICMRNFSQSGDLSEHIRTHTGEKPFACDIC |
| | | | GRKFATSGHLTTHTKIHTGSQKPFQCRICMRNF |
| | | | SDSSHLTTHIRTHTGEKPFACDICGRKFARSSH |
| | | | LTTHTKIHTGSQKPFQCRICMRNFSDRSDLTRH |
| | | | IRTHTGEKPFACDICGRKFADRSDLTRHTKIHT |
| | | | GSQKPFQCRICMRNFSRSDTLTRHIRTHTGLRG |
| | | | SNSGDPRRHSLGGSRKPDLIAYKNFDLLVIALK |
| | | | P* |
| SEQ ID | Amino | pb7 | MAAMAERPFQCRICMRNFSRSSDLTRHIRTHTG |
| NO. 39 | Acid | | EKPFACDICGRKFARSDTLTRHTKIHTGSQKPF |
| | | | QCRICMRNFSQSGDLSEHIRTHTGEKPFACDIC |
| | | | GRKFATSGHLTTHTKIHTGSQKPFQCRICMRNF |
| | | | SDSSHLTTHIRTHTGEKPFACDICGRKFARSSH |
| | | | LTTHTKIHTGSQKPFQCRICMRNFSDRSDLTRH |
| | | | IRTHTGEKPFACDICGRKFADRSDLTRHTKIHT |
| | | | GSQKPFQCRICMRNFSRSDTLTRHIRTHTGLRG |
| | | | SNSGDPRRHSLGGSRKPDLIAYKNFDLLVILLK |
| | | | P* |
| SEQ ID | Amino | pb8 | MAAMAERPFQCRICMRNESRSSDLTRHIRTHTG |
| NO. 40 | Acid | | EKPFACDICGRKFARSDTLTRHTKIHTGSQKPF |
| | | | QCRICMRNFSQSGDLSEHIRTHTGEKPFACDIC |
| | | | GRKFATSGHLTTHTKIHTGSQKPFQCRICMRNF |
| | | | SDSSHLTTHIRTHTGEKPFACDICGRKFARSSH |
| | | | LTTHTKIHTGSQKPFQCRICMRNFSDRSDLTRH |
| | | | IRTHTGEKPFACDICGRKFADRSDLTRHTKIHT |
| | | | GSQKPFQCRICMRNFSRSDTLTRHIRTHTGLRG |
| | | | SNSGDPRRHSLGGSRKPDLIAYKNFDLLVIILK |
| | | | Px |
| SEQ ID | Amino | pb9 | MAAMAERPFQCRICMRNFSRSSDLTRHIRTHTG |
| NO. 41 | Acid | | EKPFACDICGRKFARSDTLTRHTKIHTGSQKPF |
| | | | QCRICMRNFSQSGDLSEHIRTHTGEKPFACDIC |
| | | | GRKFATSGHLTTHTKIHTGSQKPFQCRICMRNF |
| | | | SDSSHLTTHIRTHTGEKPFACDICGRKFARSSH |
| | | | LTTHTKIHTGSQKPFQCRICMRNFSDRSDLTRH |
| | | | IRTHTGEKPFACDICGRKFADRSDLTRHTKIHT |
| | | | GSQKPFQCRICMRNESRSDTLTRHIRTHTGLRG |
| | | | SNSGDPRRHSLGGSRKPDLIAYKNFDLLVIHLK |
| | | | P* |
| SEQ ID | Amino | pb10 | MAAMAERPFQCRICMRNFSRSSDLTRHIRTHTG |
| NO. 42 | Acid | | EKPFACDICGRKFARSDTLTRHTKIHTGSQKPF |
| | | | QCRICMRNFSQSGDLSEHIRTHTGEKPFACDIC |
| | | | GRKFATSGHLTTHTKIHTGSQKPFQCRICMRNF |
| | | | SDSSHLTTHIRTHTGEKPFACDICGRKFARSSH |
| | | | LTTHTKIHTGSQKPFQCRICMRNFSDRSDLTRH |
| | | | IRTHTGEKPFACDICGRKFADRSDLTRHTKIHT |
| | | | GSQKPFQCRICMRNFSRSDTLTRHIRTHTGLRG |
| | | | SNSGDPRRHSLGGSRKPDLIAYKNFDLLVIRLK |
| | | | P* |
| SEQ ID | Amino | pb11 | MAAMAERPFQCRICMRNFSRSSDLTRHIRTHTG |
| NO. 43 | Acid | | EKPFACDICGRKFARSDTLTRHTKIHTGSQKPF |
| | | | QCRICMRNFSQSGDLSEHIRTHTGEKPFACDIC |
| | | | GRKFATSGHLTTHTKIHTGSQKPFQCRICMRNF |
| | | | SDSSHLTTHIRTHTGEKPFACDICGRKFARSSH |
| | | | LTTHTKIHTGSQKPFQCRICMRNFSDRSDLTRH |
| | | | IRTHTGEKPFACDICGRKFADRSDLTRHTKIHT |
| | | | GSQKPFQCRICMRNFSRSDTLTRHIRTHTGLRG |
| | | | SNSGDPRRHSLGGSRKPDLIAYKNFDLLVIKLK |
| | | | P* |
| SEQ ID | Amino | pb 12 | MAAMAERPFQCRICMRNFSRSSDLTRHIRTHTG |
| NO. 44 | Acid | | EKPFACDICGRKFARSDTLTRHTKIHTGSQKPF |
| | | | QCRICMRNFSQSGDLSEHIRTHTGEKPFACDIC |
| | | | GRKFATSGHLTTHTKIHTGSQKPFQCRICMRNF |
| | | | SDSSHLTTHIRTHTGEKPFACDICGRKFARSSH |
| | | | LTTHTKIHTGSQKPFQCRICMRNFSDRSDLTRH |
| | | | IRTHTGEKPFACDICGRKFADRSDLTRHTKIHT |
| | | | GSQKPFQCRICMRNFSRSDTLTRHIRTHTGLRG |
| | | | SNSGDPRRHSLGGSRKPDLIAYKNFDLLVIMLK |
| | | | P* |
| SEQ ID | Amino | pb16 | MAAMAERPFQCRICMRNFSRSSDLTRHIRTHTG |
| NO. 45 | Acid | | EKPFACDICGRKFARSDTLTRHTKIHTGSQKPF |
| | | | QCRICMRNFSQSGDLSEHIRTHTGEKPFACDIC |
| | | | GRKFATSGHLTTHTKIHTGSQKPFQCRICMRNF |
| | | | SDSSHLTTHIRTHTGEKPFACDICGRKFARSSH |
| | | | LTTHTKIHTGSQKPFQCRICMRNFSDRSDLTRH |
| | | | IRTHTGEKPFACDICGRKFADRSDLTRHTKIHT |
| | | | GSQKPFQCRICMRNFSRSDTLTRHIRTHTGLRG |
| | | | SNSGDPRRHSLGGSRKPNLIAYKNFDLLVIELK |
| | | | P* |
| SEQ ID | Amino | pb17 | MAAMAERPFQCRICMRNFSRSSDLTRHIRTHTG |
| NO. 46 | Acid | | EKPFACDICGRKFARSDTLTRHTKIHTGSQKPF |
| | | | QCRICMRNFSQSGDLSEHIRTHTGEKPFACDIC |
| | | | GRKFATSGHLTTHTKIHTGSQKPFQCRICMRNE |
| | | | SDSSHLTTHIRTHTGEKPFACDICGRKFARSSH |
| | | | LTTHTKIHTGSQKPFQCRICMRNESDRSDLTRH |
| | | | IRTHTGEKPFACDICGRKFADRSDLTRHTKIHT |
| | | | GSQKPFQCRICMRNFSRSDTLTRHIRTHTGLRG |
| | | | SNSGDPRRHSLGGSRKPALIAYKNEDLLVIELK |
| | | | P* |
| SEQ ID | Amino | pb 18 | MAAMAERPFQCRICMRNFSRSSDLTRHIRTHTG |
| NO. 47 | Acid | | EKPFACDICGRKFARSDTLTRHTKIHTGSQKPF |
| | | | QCRICMRNFSQSGDLSEHIRTHTGEKPFACDIC |
| | | | GRKFATSGHLTTHTKIHTGSQKPFQCRICMRNF |
| | | | SDSSHLTTHIRTHTGEKPFACDICGRKFARSSH |
| | | | LTTHTKIHTGSQKPFQCRICMRNFSDRSDLTRH |
| | | | IRTHTGEKPFACDICGRKFADRSDLTRHTKIHT |
| | | | GSQKPFQCRICMRNESRSDTLTRHIRTHTGLRG |
| | | | SNSGDPRRHSLGGSRKPDGAIYTVGSPIDYGVI |
| | | | VVTKP* |
| SEQ ID | Amino | pb19 | MAAMAERPFQCRICMRNFSRSSDLTRHIRTHTG |
| NO. 48 | Acid | | EKPFACDICGRKFARSDTLTRHTKIHTGSQKPF |
| | | | QCRICMRNFSQSGDLSEHIRTHTGEKPFACDIC |
| | | | GRKFATSGHLTTHTKIHTGSQKPFQCRICMRNF |
| | | | SDSSHLTTHIRTHTGEKPFACDICGRKFARSSH |
| | | | LTTHTKIHTGSQKPFQCRICMRNFSDRSDLTRH |
| | | | IRTHTGEKPFACDICGRKFADRSDLTRHTKIHT |
| | | | GSQKPFQCRICMRNESRSDTLTRHIRTHTGLRG |
| | | | SNSGDPRRHSLGGSRKPDFTLYKPSEPNKKIAI |
| | | | VIKP* |
| SEQ ID | Amino | pb20 | MAAMAERPFQCRICMRNFSRSSDLTRHIRTHTG |
| NO. 49 | Acid | | EKPFACDICGRKFARSDTLTRHTKIHTGSQKPF |
| | | | QCRICMRNFSQSGDLSEHIRTHTGEKPFACDIC |
| | | | GRKFATSGHLTTHTKIHTGSQKPFQCRICMRNF |
| | | | SDSSHLTTHIRTHTGEKPFACDICGRKFARSSH |
| | | | LTTHTKIHTGSQKPFQCRICMRNFSDRSDLTRH |
| | | | IRTHTGEKPFACDICGRKFADRSDLTRHTKIHT |
| | | | GSQKPFQCRICMRNFSRSDTLTRHIRTHTGLRG |
| | | | SNSGDPRRHSLGGSRKPDGLLWDDDCAIILVSK |
| | | | P* |
| SEQ ID | Amino | pb21 | MAAMAERPFQCRICMRNFSRSSDLTRHIRTHTG |
| NO. 50 | Acid | | EKPFACDICGRKFARSDTLTRHTKIHTGSQKPF |
| | | | QCRICMRNFSQSGDLSEHIRTHTGEKPFACDIC |
| | | | GRKFATSGHLTTHTKIHTGSQKPFQCRICMRNF |
| | | | SDSSHLTTHIRTHTGEKPFACDICGRKFARSSH |
| | | | LTTHTKIHTGSQKPFQCRICMRNFSDRSDLTRH |
| | | | IRTHTGEKPFACDICGRKFADRSDLTRHTKIHT |
| | | | GSQKPFQCRICMRNFSRSDTLTRHIRTHTGLRG |
| | | | SNSGDPRRHSLGGSRKPDHIYQLVYNSTDTLLL |
| | | | IVSKP* |
| SEQ ID | Amino | pb22 | MAAMAERPFQCRICMRNFSRSSDLTRHIRTHTG |
| NO. 51 | Acid | | EKPFACDICGRKFARSDTLTRHTKIHTGSQKPF |
| | | | QCRICMRNFSQSGDLSEHIRTHTGEKPFACDIC |
| | | | GRKFATSGHLTTHTKIHTGSQKPFQCRICMRNF |
| | | | SDSSHLTTHIRTHTGEKPFACDICGRKFARSSH |
| | | | LTTHTKIHTGSQKPFQCRICMRNFSDRSDLTRH |
| | | | IRTHTGEKPFACDICGRKFADRSDLTRHTKIHT |
| | | | GSQKPFQCRICMRNFSRSDTLTRHIRTHTGLRG |
| | | | SNSGDPRRHSLGGSRKPDHIYIFNDDNNTKNGL |
| | | | IIVSKP* |
| SEQ ID | Amino | pb23 | MAAMAERPFQCRICMRNESRSSDLTRHIRTHTG |
| NO. 52 | Acid | | EKPFACDICGRKFARSDTLTRHTKIHTGSQKPF |
| | | | QCRICMRNFSQSGDLSEHIRTHTGEKPFACDIC |
| | | | GRKFATSGHLTTHTKIHTGSQKPFQCRICMRNF |
| | | | SDSSHLTTHIRTHTGEKPFACDICGRKFARSSH |
| | | | LTTHTKIHTGSQKPFQCRICMRNFSDRSDLTRH |
| | | | IRTHTGEKPFACDICGRKFADRSDLTRHTKIHT |
| | | | GSQKPFQCRICMRNFSRSDTLTRHIRTHTGLRG |
| | | | SNSGDPRRHSLGGSRKPDHVIQILDLFEKPLLL |
| | | | SIVSKP* |
| SEQ ID | Amino | pb24 | MAAMAERPFQCRICMRNFSRSSDLTRHIRTHTG |
| NO. 53 | Acid | | EKPFACDICGRKFARSDTLTRHTKIHTGSQKPF |
| | | | QCRICMRNFSQSGDLSEHIRTHTGEKPFACDIC |
| | | | GRKFATSGHLTTHTKIHTGSQKPFQCRICMRNF |
| | | | SDSSHLTTHIRTHTGEKPFACDICGRKFARSSH |
| | | | LTTHTKIHTGSQKPFQCRICMRNESDRSDLTRH |
| | | | IRTHTGEKPFACDICGRKFADRSDLTRHTKIHT |
| | | | GSQKPFQCRICMRNFSRSDTLTRHIRTHTGLRG |
| | | | SNSGDPRRHSLGGSRKPDIILVNDNISLILILV |
| | | | AKP* |
| SEQ ID | Amino | pb25 | MAAMAERPFQCRICMRNFSRSSDLTRHIRTHTG |
| NO. 54 | Acid | | EKPFACDICGRKFARSDTLTRHTKIHTGSQKPF |
| | | | QCRICMRNFSQSGDLSEHIRTHTGEKPFACDIC |
| | | | GRKFATSGHLTTHTKIHTGSQKPFQCRICMRNF |
| | | | SDSSHLTTHIRTHTGEKPFACDICGRKFARSSH |
| | | | LTTHTKIHTGSQKPFQCRICMRNFSDRSDLTRH |
| | | | IRTHTGEKPFACDICGRKFADRSDLTRHTKIHT |
| | | | GSQKPFQCRICMRNFSRSDTLTRHIRTHTGLRG |
| | | | SNSGDPRRHSLGGSRKPDLIAYKNDDLLVIVAK |
| | | | P* |
| SEQ ID | Amino | pb26 | MAAMAERPFQCRICMRNFSRSSDLTRHIRTHTG |
| NO. 55 | Acid | | EKPFACDICGRKFARSDTLTRHTKIHTGSQKPF |
| | | | QCRICMRNFSQSGDLSEHIRTHTGEKPFACDIC |
| | | | GRKFATSGHLTTHTKIHTGSQKPFQCRICMRNF |
| | | | SDSSHLTTHIRTHTGEKPFACDICGRKFARSSH |
| | | | LTTHTKIHTGSQKPFQCRICMRNFSDRSDLTRH |
| | | | IRTHTGEKPFACDICGRKFADRSDLTRHTKIHT |
| | | | GSQKPFQCRICMRNFSRSDTLTRHIRTHTGLRG |
| | | | SNSGDPRRHSLGKIVPALIAYKNFDLLVIELKP |
| | | | * |
| SEQ ID | Amino | pb27 | MAAMAERPFQCRICMRNFSRSSDLTRHIRTHTG |
| NO. 56 | Acid | | EKPFACDICGRKFARSDTLTRHTKIHTGSQKPF |
| | | | QCRICMRNFSQSGDLSEHIRTHTGEKPFACDIC |
| | | | GRKFATSGHLTTHTKIHTGSQKPFQCRICMRNF |
| | | | SDSSHLTTHIRTHTGEKPFACDICGRKFARSSH |
| | | | LTTHTKIHTGSQKPFQCRICMRNFSDRSDLTRH |
| | | | IRTHTGEKPFACDICGRKFADRSDLTRHTKIHT |
| | | | GSQKPFQCRICMRNFSRSDTLTRHIRTHTGLRG |
| | | | SNSGDPRRHSLGGSNKPALIAYKNFDLLVIELK |
| | | | P* |
| SEQ ID | Amino | pb28 | MAAMAERPFQCRICMRNFSRSSDLTRHIRTHTG |
| NO. 57 | Acid | | EKPFACDICGRKFARSDTLTRHTKIHTGSQKPF |
| | | | QCRICMRNFSQSGDLSEHIRTHTGEKPFACDIC |
| | | | GRKFATSGHLTTHTKIHTGSQKPFQCRICMRNF |
| | | | SDSSHLTTHIRTHTGEKPFACDICGRKFARSSH |
| | | | LTTHTKIHTGSQKPFQCRICMRNFSDRSDLTRH |
| | | | IRTHTGEKPFACDICGRKFADRSDLTRHTKIHT |
| | | | GSQKPFQCRICMRNFSRSDTLTRHIRTHTGLRG |
| | | | SNSGDPRRHSLGTKRPALIAYKNEDLLVIELKP |
| | | | * |
| SEQ ID | Amino | pb29 | MAAMAERPFQCRICMRNFSRSSDLTRHIRTHTG |
| NO. 58 | Acid | | EKPFACDICGRKFARSDTLTRHTKIHTGSQKPF |
| | | | QCRICMRNFSQSGDLSEHIRTHTGEKPFACDIC |
| | | | GRKFATSGHLTTHTKIHTGSQKPFQCRICMRNF |
| | | | SDSSHLTTHIRTHTGEKPFACDICGRKFARSSH |
| | | | LTTHTKIHTGSQKPFQCRICMRNFSDRSDLTRH |
| | | | IRTHTGEKPFACDICGRKFADRSDLTRHTKIHT |
| | | | GSQKPFQCRICMRNFSRSDTLTRHIRTHTGLRG |
| | | | SNSGDPRRHSLGGETKRPALIAYKNFDLLVIEL |
| | | | KP* |
| SEQ ID | Amino | pb30 | MAAMAERPFQCRICMRNFSRSSDLTRHIRTHTG |
| NO. 59 | Acid | | EKPFACDICGRKFARSDTLTRHTKIHTGSQKPF |
| | | | QCRICMRNFSQSGDLSEHIRTHTGEKPFACDIC |
| | | | GRKFATSGHLTTHTKIHTGSQKPFQCRICMRNF |
| | | | SDSSHLTTHIRTHTGEKPFACDICGRKFARSSH |
| | | | LTTHTKIHTGSQKPFQCRICMRNFSDRSDLTRH |
| | | | IRTHTGEKPFACDICGRKFADRSDLTRHTKIHT |
| | | | GSQKPFQCRICMRNFSRSDTLTRHIRTHTGLRG |
| | | | SNSGDPRRHSLGGKRPALIAYKNFDLLVIELKP |
| | | | * |
| SEQ ID | Amino | pb31 | MAAMAERPFQCRICMRNFSRSSDLTRHIRTHTG |
| NO. 60 | Acid | | EKPFACDICGRKFARSDTLTRHTKIHTGSQKPF |
| | | | QCRICMRNFSQSGDLSEHIRTHTGEKPFACDIC |
| | | | GRKFATSGHLTTHTKIHTGSQKPFQCRICMRNF |
| | | | SDSSHLTTHIRTHTGEKPFACDICGRKFARSSH |
| | | | LTTHTKIHTGSQKPFQCRICMRNFSDRSDLTRH |
| | | | IRTHTGEKPFACDICGRKFADRSDLTRHTKIHT |
| | | | GSQKPFQCRICMRNFSRSDTLTRHIRTHTGLRG |
| | | | SNSGDPRRHSLGREDERPALIAYKNFDLLVIEL |
| | | | KP* |
| SEQ ID | RNA | Pop45-crRNA (967- | mG*mA*GCUGGACGGGGACGUAAAGUUUUAGAG |
| NO. 61 | | 990 EGFPDP2) | CUAUG*mC*mU |
| SEQ ID | DNA | EGFP2 targeting | GGAGCTGGACGGGGACGTAAACGG |
| NO. 62 | | site of dCAS9 | |
| SEQ ID | Amino | dcas9-linker-R | DKKYSIGLAIGTNSVGWAVITDEYKVPSKKFKV |
| NO. 63 | Acid | | LGNTDRHSIKKNLIGALLFDSGETAEATRLKRT |
| | | | ARRRYTRRKNRICYLQEIFSNEMAKVDDSFFHR |
| | | | LEESFLVEEDKKHERHPIFGNIVDEVAYHEKYP |
| | | | TIYHLRKKLVDSTDKADLRLIYLALAHMIKFRG |
| | | | HFLIEGDLNPDNSDVDKLFIQLVQTYNQLFEEN |
| | | | PINASGVDAKAILSARLSKSRRLENLIAQLPGE |
| | | | KKNGLFGNLIALSLGLTPNFKSNFDLAEDAKLQ |
| | | | LSKDTYDDDLDNLLAQIGDQYADLFLAAKNLSD |
| | | | AILLSDILRVNTEITKAPLSASMIKRYDEHHQD |
| | | | LTLLKALVRQQLPEKYKEIFFDQSKNGYAGYID |
| | | | GGASQEEFYKFIKPILEKMDGTEELLVKLNRED |
| | | | LLRKQRTFDNGSIPHQIHLGELHAILRRQEDFY |
| | | | PFLKDNREKIEKILTFRIPYYVGPLARGNSRFA |
| | | | WMTRKSEETITPWNFEEVVDKGASAQSFIERMT |
| | | | NFDKNLPNEKVLPKHSLLYEYFTVYNELTKVKY |
| | | | VTEGMRKPAFLSGEQKKAIVDLLFKTNRKVTVK |
| | | | QLKEDYFKKIECFDSVEISGVEDRENASLGTYH |
| | | | DLLKIIKDKDELDNEENEDILEDIVLTLTLFED |
| | | | REMIEERLKTYAHLFDDKVMKQLKRRRYTGWGR |
| | | | LSRKLINGIRDKQSGKTILDFLKSDGFANRNEM |
| | | | QLIHDDSLTFKEDIQKAQVSGQGDSLHEHIANL |
| | | | AGSPAIKKGILQTVKVVDELVKVMGRHKPENIV |
| | | | IEMARENQTTQKGQKNSRERMKRIEEGIKELGS |
| | | | QILKEHPVENTQLQNEKLYLYYLQNGRDMYVDQ |
| | | | ELDINRLSDYDVDAIVPQSFLKDDSIDNKVLTR |
| | | | SDKNRGKSDNVPSEEVVKKMKNYWRQLLNAKLI |
| | | | TQRKFDNLTKAERGGLSELDKAGFIKRQLVETR |
| | | | QITKHVAQILDSRMNTKYDENDKLIREVKVITL |
| | | | KSKLVSDFRKDFQFYKVREINNYHHAHDAYLNA |
| | | | VVGTALIKKYPKLESEFVYGDYKVYDVRKMIAK |
| | | | SEQEIGKATAKYFFYSNIMNFFKTEITLANGEI |
| | | | RKRPLIETNGETGEIVWDKGRDFATVRKVLSMP |
| | | | QVNIVKKTEVQTGGFSKESILPKRNSDKLIARK |
| | | | KDWDPKKYGGFDSPTVAYSVLVVAKVEKGKSKK |
| | | | LKSVKELLGITIMERSSFEKNPIDFLEAKGYKE |
| | | | VKKDLIIKLPKYSLFELENGRKRMLASAGELQK |
| | | | GNELALPSKYVNFLYLASHYEKLKGSPEDNEQK |
| | | | QLFVEQHKHYLDEIIEQISEFSKRVILADANLD |
| | | | KVLSAYNKHRDKPIREQAENIIHLFTLTNLGAP |
| | | | AAFKYFDTTIDRKRYTSTKEVLDATLIHQSITG |
| | | | LYETRIDLSQLGGDLRQKDAARGSNSGDPRRHS |
| | | | LGGSRKPDLIAYKNFDLLVIVLKP* |
| SEQ ID | DNA | dcas9-linker-R | GACAAGAAGTACAGCATCGGCCTGGCCATCGGC |
| NO. 64 | | | ACCAACTCTGTGGGCTGGGCCGTGATCACCGAC |
| | | | GAGTACAAGGTGCCCAGCAAGAAATTCAAGGTG |
| | | | CTGGGCAACACCGACCGGCACAGCATCAAGAAG |
| | | | AACCTGATCGGAGCCCTGCTGTTCGACAGCGGC |
| | | | GAAACAGCCGAGGCCACCCGGCTGAAGAGAACC |
| | | | GCCAGAAGAAGATACACCAGACGGAAGAACCGG |
| | | | ATCTGCTATCTGCAAGAGATCTTCAGCAACGAG |
| | | | ATGGCCAAGGTGGACGACAGCTTCTTCCACAGA |
| | | | CTGGAAGAGTCCTTCCTGGTGGAAGAGGATAAG |
| | | | AAGCACGAGCGGCACCCCATCTTCGGCAACATC |
| | | | GTGGACGAGGTGGCCTACCACGAGAAGTACCCC |
| | | | ACCATCTACCACCTGAGAAAGAAACTGGTGGAC |
| | | | AGCACCGACAAGGCCGACCTGCGGCTGATCTAT |
| | | | CTGGCCCTGGCCCACATGATCAAGTTCCGGGGC |
| | | | CACTTCCTGATCGAGGGCGACCTGAACCCCGAC |
| | | | AACAGCGACGTGGACAAGCTGTTCATCCAGCTG |
| | | | GTGCAGACCTACAACCAGCTGTTCGAGGAAAAC |
| | | | CCCATCAACGCCAGCGGCGTGGACGCCAAGGCC |
| | | | ATCCTGTCTGCCAGACTGAGCAAGAGCAGACGG |
| | | | CTGGAAAATCTGATCGCCCAGCTGCCCGGCGAG |
| | | | AAGAAGAATGGCCTGTTCGGCAACCTGATTGCC |
| | | | CTGAGCCTGGGCCTGACCCCCAACTTCAAGAGC |
| | | | AACTTCGACCTGGCCGAGGATGCCAAACTGCAG |
| | | | CTGAGCAAGGACACCTACGACGACGACCTGGAC |
| | | | AACCTGCTGGCCCAGATCGGCGACCAGTACGCC |
| | | | GACCTGTTTCTGGCCGCCAAGAACCTGTCCGAC |
| | | | GCCATCCTGCTGAGCGACATCCTGAGAGTGAAC |
| | | | ACCGAGATCACCAAGGCCCCCCTGAGCGCCTCT |
| | | | ATGATCAAGAGATACGACGAGCACCACCAGGAC |
| | | | CTGACCCTGCTGAAAGCTCTCGTGCGGCAGCAG |
| | | | CTGCCTGAGAAGTACAAAGAGATTTTCTTCGAC |
| | | | CAGAGCAAGAACGGCTACGCCGGCTACATTGAC |
| | | | GGCGGAGCCAGCCAGGAAGAGTTCTACAAGTTC |
| | | | ATCAAGCCCATCCTGGAAAAGATGGACGGCACC |
| | | | GAGGAACTGCTCGTGAAGCTGAACAGAGAGGAC |
| | | | CTGCTGCGGAAGCAGCGGACCTTCGACAACGGC |
| | | | AGCATCCCCCACCAGATCCACCTGGGAGAGCTG |
| | | | CACGCCATTCTGCGGCGGCAGGAAGATTTTTAC |
| | | | CCATTCCTGAAGGACAACCGGGAAAAGATCGAG |
| | | | AAGATCCTGACCTTCCGCATCCCCTACTACGTG |
| | | | GGCCCTCTGGCCAGGGGAAACAGCAGATTCGCC |
| | | | TGGATGACCAGAAAGAGCGAGGAAACCATCACC |
| | | | CCCTGGAACTTCGAGGAAGTGGTGGACAAGGGC |
| | | | GCTTCCGCCCAGAGCTTCATCGAGCGGATGACC |
| | | | AACTTCGATAAGAACCTGCCCAACGAGAAGGTG |
| | | | CTGCCCAAGCACAGCCTGCTGTACGAGTACTTC |
| | | | ACCGTGTATAACGAGCTGACCAAAGTGAAATAC |
| | | | GTGACCGAGGGAATGAGAAAGCCCGCCTTCCTG |
| | | | AGCGGCGAGCAGAAAAAGGCCATCGTGGACCTG |
| | | | CTGTTCAAGACCAACCGGAAAGTGACCGTGAAG |
| | | | CAGCTGAAAGAGGACTACTTCAAGAAAATCGAG |
| | | | TGCTTCGACTCCGTGGAAATCTCCGGCGTGGAA |
| | | | GATCGGTTCAACGCCTCCCTGGGCACATACCAC |
| | | | GATCTGCTGAAAATTATCAAGGACAAGGACTTC |
| | | | CTGGACAATGAGGAAAACGAGGACATTCTGGAA |
| | | | GATATCGTGCTGACCCTGACACTGTTTGAGGAC |
| | | | AGAGAGATGATCGAGGAACGGCTGAAAACCTAT |
| | | | GCCCACCTGTTCGACGACAAAGTGATGAAGCAG |
| | | | CTGAAGCGGCGGAGATACACCGGCTGGGGCAGG |
| | | | CTGAGCCGGAAGCTGATCAACGGCATCCGGGAC |
| | | | AAGCAGTCCGGCAAGACAATCCTGGATTTCCTG |
| | | | AAGTCCGACGGCTTCGCCAACAGAAACTTCATG |
| | | | CAGCTGATCCACGACGACAGCCTGACCTTTAAA |
| | | | GAGGACATCCAGAAAGCCCAGGTGTCCGGCCAG |
| | | | GGCGATAGCCTGCACGAGCACATTGCCAATCTG |
| | | | GCCGGCAGCCCCGCCATTAAGAAGGGCATCCTG |
| | | | CAGACAGTGAAGGTGGTGGACGAGCTCGTGAAA |
| | | | GTGATGGGCCGGCACAAGCCCGAGAACATCGTG |
| | | | ATCGAAATGGCCAGAGAGAACCAGACCACCCAG |
| | | | AAGGGACAGAAGAACAGCCGCGAGAGAATGAAG |
| | | | CGGATCGAAGAGGGCATCAAAGAGCTGGGCAGC |
| | | | CAGATCCTGAAAGAACACCCCGTGGAAAACACC |
| | | | CAGCTGCAGAACGAGAAGCTGTACCTGTACTAC |
| | | | CTGCAGAATGGGCGGGATATGTACGTGGACCAG |
| | | | GAACTGGACATCAACCGGCTGTCCGACTACGAT |
| | | | GTGGACGCCATCGTGCCTCAGAGCTTTCTGAAG |
| | | | GACGACTCCATCGACAACAAGGTGCTGACCAGA |
| | | | AGCGACAAGAACCGGGGCAAGAGCGACAACGTG |
| | | | CCCTCCGAAGAGGTCGTGAAGAAGATGAAGAAC |
| | | | TACTGGCGGCAGCTGCTGAACGCCAAGCTGATT |
| | | | ACCCAGAGAAAGTTCGACAATCTGACCAAGGCC |
| | | | GAGAGAGGCGGCCTGAGCGAACTGGATAAGGCC |
| | | | GGCTTCATCAAGAGACAGCTGGTGGAAACCCGG |
| | | | CAGATCACAAAGCACGTGGCACAGATCCTGGAC |
| | | | TCCCGGATGAACACTAAGTACGACGAGAATGAC |
| | | | AAGCTGATCCGGGAAGTGAAAGTGATCACCCTG |
| | | | AAGTCCAAGCTGGTGTCCGATTTCCGGAAGGAT |
| | | | TTCCAGTTTTACAAAGTGCGCGAGATCAACAAC |
| | | | TACCACCACGCCCACGACGCCTACCTGAACGCC |
| | | | GTCGTGGGAACCGCCCTGATCAAAAAGTACCCT |
| | | | AAGCTGGAAAGCGAGTTCGTGTACGGCGACTAC |
| | | | AAGGTGTACGACGTGCGGAAGATGATCGCCAAG |
| | | | AGCGAGCAGGAAATCGGCAAGGCTACCGCCAAG |
| | | | TACTTCTTCTACAGCAACATCATGAACTTTTTC |
| | | | AAGACCGAGATTACCCTGGCCAACGGCGAGATC |
| | | | CGGAAGCGGCCTCTGATCGAGACAAACGGCGAA |
| | | | ACCGGGGAGATCGTGTGGGATAAGGGCCGGGAT |
| | | | TTTGCCACCGTGCGGAAAGTGCTGAGCATGCCC |
| | | | CAAGTGAATATCGTGAAAAAGACCGAGGTGCAG |
| | | | ACAGGCGGCTTCAGCAAAGAGTCTATCCTGCCC |
| | | | AAGAGGAACAGCGATAAGCTGATCGCCAGAAAG |
| | | | AAGGACTGGGACCCTAAGAAGTACGGCGGCTTC |
| | | | GACAGCCCCACCGTGGCCTATTCTGTGCTGGTG |
| | | | GTGGCCAAAGTGGAAAAGGGCAAGTCCAAGAAA |
| | | | CTGAAGAGTGTGAAAGAGCTGCTGGGGATCACC |
| | | | ATCATGGAAAGAAGCAGCTTCGAGAAGAATCCC |
| | | | ATCGACTTTCTGGAAGCCAAGGGCTACAAAGAA |
| | | | GTGAAAAAGGACCTGATCATCAAGCTGCCTAAG |
| | | | TACTCCCTGTTCGAGCTGGAAAACGGCCGGAAG |
| | | | AGAATGCTGGCCTCTGCCGGCGAACTGCAGAAG |
| | | | GGAAACGAACTGGCCCTGCCCTCCAAATATGTG |
| | | | AACTTCCTGTACCTGGCCAGCCACTATGAGAAG |
| | | | CTGAAGGGCTCCCCCGAGGATAATGAGCAGAAA |
| | | | CAGCTGTTTGTGGAACAGCACAAGCACTACCTG |
| | | | GACGAGATCATCGAGCAGATCAGCGAGTTCTCC |
| | | | AAGAGAGTGATCCTGGCCGACGCTAATCTGGAC |
| | | | AAAGTGCTGTCCGCCTACAACAAGCACCGGGAT |
| | | | AAGCCCATCAGAGAGCAGGCCGAGAATATCATC |
| | | | CACCTGTTTACCCTGACCAATCTGGGAGCCCCT |
| | | | GCCGCCTTCAAGTACTTTGACACCACCATCGAC |
| | | | CGGAAGAGGTACACCAGCACCAAAGAGGTGCTG |
| | | | GACGCCACCCTGATCCACCAGAGCATCACCGGC |
| | | | CTGTACGAGACACGGATCGACCTGTCTCAGCTG |
| | | | GGAGGCGACCTGAGACAGAAGGACGCCGCCCGG |
| | | | GGATCCAATTCTGGTGATCCTCGGAGACACAGT |
| | | | CTGGGCGGTTCTCGTAAACCCGATCTGATTGCC |
| | | | TATAAAAACTTTGATCTGCTGGTCATTGTTCTT |
| | | | AAGCCTTGA |
| SEQ ID | Amino | Linker Seq for | LRQKDAARGS |
| NO. 65 | Acid | dCas9 | |
| SEQ ID | DNA | pb42 plasmid full | GACTCTTCGCGATGTACGGGCCAGATATACGCG |
| NO. 66 | | length DNA | TTGACATTGATTATTGACTAGTTATTAATAGTA |
| | | sequence | ATCAATTACGGGGTCATTAGTTCATAGCCCATA |
| | | | TATGGAGTTCCGCGTTACATAACTTACGGTAAA |
| | | | TGGCCCGCCTGGCTGACCGCCCAACGACCCCCG |
| | | | CCCATTGACGTCAATAATGACGTATGTTCCCAT |
| | | | AGTAACGCCAATAGGGACTTTCCATTGACGTCA |
| | | | ATGGGTGGACTATTTACGGTAAACTGCCCACTT |
| | | | GGCAGTACATCAAGTGTATCATATGCCAAGTAC |
| | | | GCCCCCTATTGACGTCAATGACGGTAAATGGCC |
| | | | CGCCTGGCATTATGCCCAGTACATGACCTTATG |
| | | | GGACTTTCCTACTTGGCAGTACATCTACGTATT |
| | | | AGTCATCGCTATTACCATGGTGATGCGGTTTTG |
| | | | GCAGTACATCAATGGGCGTGGATAGCGGTTTGA |
| | | | CTCACGGGGATTTCCAAGTCTCCACCCCATTGA |
| | | | CGTCAATGGGAGTTTGTTTTGGCACCAAAATCA |
| | | | ACGGGACTTTCCAAAATGTCGTAACAACTCCGC |
| | | | CCCATTGACGCAAATGGGCGGTAGGCGTGTACG |
| | | | GTGGGAGGTCTATATAAGCAGAGCTCTCTGGCT |
| | | | AACTAGAGAACCCACTGCTTACTGGCTTATCGA |
| | | | AATTAATACGACTCACTATAGGGAGACCCAAGC |
| | | | TGGCTAGCGTTTAAACTTAAGCTTATGGACTAC |
| | | | AAAGACCATGACGGTGATTATAAAGATCATGAC |
| | | | ATCGATTACAAGGATGACGATGACAAGATGGCC |
| | | | CCCAAGAAGAAGAGGAAGGTGGGCATTCACGGG |
| | | | GTACCGGCGGCGATGGCCGAGCGGCCCTTCCAG |
| | | | TGCAGGATCTGTATGCGCAACTTTTCTCGTTCT |
| | | | TCTGCTCTTACTCGTCACATCAGAACCCATACA |
| | | | GGCGAAAAGCCTTTCGCCTGCGACATTTGTGGG |
| | | | AGAAAATTTGCTCGTTCTGATACTCTTACTCGT |
| | | | CATACCAAGATCCACACCGGCTCTCAGAAACCA |
| | | | TTCCAGTGCCGCATTTGTATGCGGAATTTTTCC |
| | | | GATCGTTCTAATCTTACTCGTCATATCCGCACT |
| | | | CACACCGGAGAGAAGCCCTTTGCTTGCGACATT |
| | | | TGTGGCAGGAAATTTGCTCGTTCTGATAATCTT |
| | | | ACTCGTCACACTAAGATCCATACTGGGTCACAG |
| | | | AAACCTTTCCAGTGCCGGATTTGTATGAGAAAC |
| | | | TTTAGCCGTTCTGATCATCTTACTCGTCACATC |
| | | | AGAACACATACTGGGCTGAGAGGATCCGGCGGC |
| | | | GGCGGCGGCTCCGGCGGCGGCGGCGGCTCCGGC |
| | | | GGCGGCGGCGGCTCCGGCGGCGGCGGCGGCTCC |
| | | | GGCGGCGGCGGCGGCTCCGGCGGCGGCGGCGGC |
| | | | TCCATGGCCGAGCGGCCCTTCCAGTGCAGGATC |
| | | | TGTATGCGCAACTTTTCCGATCGTTCTAATCTT |
| | | | ACTCGTCACATCAGAACCCATACAGGCGAAAAG |
| | | | CCTTTCGCCTGCGACATTTGTGGGCGGAAATTT |
| | | | GCTCGTTCTGATCATCTTACTCGTCACACAAAG |
| | | | ATCCATACTGGCAGCCAGAAACCATTCCAGTGC |
| | | | AGGATTTGCATGAGAAACTTTTCCGATCGTTCT |
| | | | AATCTTACTCGTCACATCCGCACTCATACCGGA |
| | | | GAGAAGCCCTTTGCTTGCGACATTTGTGGCCGG |
| | | | AAATTTGCTCGTTCTGATTCTCTTTCTGAACAT |
| | | | ACAAAGATCCATACTGGGTCTCAGAAACCTTTC |
| | | | CAGTGCAGGATTTGTATGAGAAATTTTTCCCGT |
| | | | TCTTCTAATCTTACTCGTCACATCAGAACACAT |
| | | | ACTGGGGAGAAGCCCTTTGCATGCGACATTTGT |
| | | | GGACGGAAATTTGCTCGTTCTGATTCTCTTACT |
| | | | CGTCATACCAAGATTCACTGAGCGGCCGCTCGA |
| | | | GTCTAGAGGGCCCGTTTAAACCCGCTGATCAGC |
| | | | CTCGACTGTGCCTTCTAGTTGCCAGCCATCTGT |
| | | | TGTTTGCCCCTCCCCCGTGCCTTCCTTGACCCT |
| | | | GGAAGGTGCCACTCCCACTGTCCTTTCCTAATA |
| | | | AAATGAGGAAATTGCATCGCATTGTCTGAGTAG |
| | | | GTGTCATTCTATTCTGGGGGGTGGGGTGGGGCA |
| | | | GGACAGCAAGGGGGAGGATTGGGAAGACAATAG |
| | | | CAGGCATGCTGGGGATGCGGTGGGCTCTATGGC |
| | | | TTCTACTGGGCGGTTTTATGGACAGCAAGCGAA |
| | | | CCGGAATTGCCAGCTGGGGCGCCCTCTGGTAAG |
| | | | GTTGGGAAGCCCTGCAAAGTAAACTGGATGGCT |
| | | | TTCTCGCCGCCAAGGATCTGATGGCGCAGGGGA |
| | | | TCAAGCTCTGATCAAGAGACAGGATGAGGATCG |
| | | | TTTCGCATGATTGAACAAGATGGATTGCACGCA |
| | | | GGTTCTCCGGCCGCTTGGGTGGAGAGGCTATTC |
| | | | GGCTATGACTGGGCACAACAGACAATCGGCTGC |
| | | | TCTGATGCCGCCGTGTTCCGGCTGTCAGCGCAG |
| | | | GGGCGCCCGGTTCTTTTTGTCAAGACCGACCTG |
| | | | TCCGGTGCCCTGAATGAACTGCAAGACGAGGCA |
| | | | GCGCGGCTATCGTGGCTGGCCACGACGGGCGTT |
| | | | CCTTGCGCAGCTGTGCTCGACGTTGTCACTGAA |
| | | | GCGGGAAGGGACTGGCTGCTATTGGGCGAAGTG |
| | | | CCGGGGCAGGATCTCCTGTCATCTCACCTTGCT |
| | | | CCTGCCGAGAAAGTATCCATCATGGCTGATGCA |
| | | | ATGCGGCGGCTGCATACGCTTGATCCGGCTACC |
| | | | TGCCCATTCGACCACCAAGCGAAACATCGCATC |
| | | | GAGCGAGCACGTACTCGGATGGAAGCCGGTCTT |
| | | | GTCGATCAGGATGATCTGGACGAAGAGCATCAG |
| | | | GGGCTCGCGCCAGCCGAACTGTTCGCCAGGCTC |
| | | | AAGGCGAGCATGCCCGACGGCGAGGATCTCGTC |
| | | | GTGACCCATGGCGATGCCTGCTTGCCGAATATC |
| | | | ATGGTGGAAAATGGCCGCTTTTCTGGATTCATC |
| | | | GACTGTGGCCGGCTGGGTGTGGCGGACCGCTAT |
| | | | CAGGACATAGCGTTGGCTACCCGTGATATTGCT |
| | | | GAAGAGCTTGGCGGCGAATGGGCTGACCGCTTC |
| | | | CTCGTGCTTTACGGTATCGCCGCTCCCGATTCG |
| | | | CAGCGCATCGCCTTCTATCGCCTTCTTGACGAG |
| | | | TTCTTCTGAATTATTAACGCTTACAATTTCCTG |
| | | | ATGCGGTATTTTCTCCTTACGCATCTGTGCGGT |
| | | | ATTTCACACCGCATACAGGTGGCACTTTTCGGG |
| | | | GAAATGTGCGCGGAACCCCTATTTGTTTATTTT |
| | | | TCTAAATACATTCAAATATGTATCCGCTCATGA |
| | | | GACAATAACCCTGATAAATGCTTCAATAATAGC |
| | | | ACGTGCTAAAACTTCATTTTTAATTTAAAAGGA |
| | | | TCTAGGTGAAGATCCTTTTTGATAATCTCATGA |
| | | | CCAAAATCCCTTAACGTGAGTTTTCGTTCCACT |
| | | | GAGCGTCAGACCCCGTAGAAAAGATCAAAGGAT |
| | | | CTTCTTGAGATCCTTTTTTTCTGCGCGTAATCT |
| | | | GCTGCTTGCAAACAAAAAAACCACCGCTACCAG |
| | | | CGGTGGTTTGTTTGCCGGATCAAGAGCTACCAA |
| | | | CTCTTTTTCCGAAGGTAACTGGCTTCAGCAGAG |
| | | | CGCAGATACCAAATACTGTCCTTCTAGTGTAGC |
| | | | CGTAGTTAGGCCACCACTTCAAGAACTCTGTAG |
| | | | CACCGCCTACATACCTCGCTCTGCTAATCCTGT |
| | | | TACCAGTGGCTGCTGCCAGTGGCGATAAGTCGT |
| | | | GTCTTACCGGGTTGGACTCAAGACGATAGTTAC |
| | | | CGGATAAGGCGCAGCGGTCGGGCTGAACGGGGG |
| | | | GTTCGTGCACACAGCCCAGCTTGGAGCGAACGA |
| | | | CCTACACCGAACTGAGATACCTACAGCGTGAGC |
| | | | TATGAGAAAGCGCCACGCTTCCCGAAGGGAGAA |
| | | | AGGCGGACAGGTATCCGGTAAGCGGCAGGGTCG |
| | | | GAACAGGAGAGCGCACGAGGGAGCTTCCAGGGG |
| | | | GAAACGCCTGGTATCTTTATAGTCCTGTCGGGT |
| | | | TTCGCCACCTCTGACTTGAGCGTCGATTTTTGT |
| | | | GATGCTCGTCAGGGGGGCGGAGCCTATGGAAAA |
| | | | ACGCCAGCAACGCGGCCTTTTTACGGTTCCTGG |
| | | | GCTTTTGCTGGCCTTTTGCTCACATGTTCTT |
| SEQ ID | DNA | pb42 cDNA | ATGGACTACAAAGACCATGACGGTGATTATAAA |
| NO. 67 | | | GATCATGACATCGATTACAAGGATGACGATGAC |
| | | | AAGATGGCCCCCAAGAAGAAGAGGAAGGTGGGC |
| | | | ATTCACGGGGTACCGGCGGCGATGGCCGAGCGG |
| | | | CCCTTCCAGTGCAGGATCTGTATGCGCAACTTT |
| | | | TCTCGTTCTTCTGCTCTTACTCGTCACATCAGA |
| | | | ACCCATACAGGCGAAAAGCCTTTCGCCTGCGAC |
| | | | ATTTGTGGGAGAAAATTTGCTCGTTCTGATACT |
| | | | CTTACTCGTCATACCAAGATCCACACCGGCTCT |
| | | | CAGAAACCATTCCAGTGCCGCATTTGTATGCGG |
| | | | AATTTTTCCGATCGTTCTAATCTTACTCGTCAT |
| | | | ATCCGCACTCACACCGGAGAGAAGCCCTTTGCT |
| | | | TGCGACATTTGTGGCAGGAAATTTGCTCGTTCT |
| | | | GATAATCTTACTCGTCACACTAAGATCCATACT |
| | | | GGGTCACAGAAACCTTTCCAGTGCCGGATTTGT |
| | | | ATGAGAAACTTTAGCCGTTCTGATCATCTTACT |
| | | | CGTCACATCAGAACACATACTGGGCTGAGAGGA |
| | | | TCCGGCGGCGGCGGCGGCTCCGGCGGCGGCGGC |
| | | | GGCTCCGGCGGCGGCGGCGGCTCCGGCGGCGGC |
| | | | GGCGGCTCCGGCGGCGGCGGCGGCTCCGGCGGC |
| | | | GGCGGCGGCTCCATGGCCGAGCGGCCCTTCCAG |
| | | | TGCAGGATCTGTATGCGCAACTTTTCCGATCGT |
| | | | TCTAATCTTACTCGTCACATCAGAACCCATACA |
| | | | GGCGAAAAGCCTTTCGCCTGCGACATTTGTGGG |
| | | | CGGAAATTTGCTCGTTCTGATCATCTTACTCGT |
| | | | CACACAAAGATCCATACTGGCAGCCAGAAACCA |
| | | | TTCCAGTGCAGGATTTGCATGAGAAACTTTTCC |
| | | | GATCGTTCTAATCTTACTCGTCACATCCGCACT |
| | | | CATACCGGAGAGAAGCCCTTTGCTTGCGACATT |
| | | | TGTGGCCGGAAATTTGCTCGTTCTGATTCTCTT |
| | | | TCTGAACATACAAAGATCCATACTGGGTCTCAG |
| | | | AAACCTTTCCAGTGCAGGATTTGTATGAGAAAT |
| | | | TTTTCCCGTTCTTCTAATCTTACTCGTCACATC |
| | | | AGAACACATACTGGGGAGAAGCCCTTTGCATGC |
| | | | GACATTTGTGGACGGAAATTTGCTCGTTCTGAT |
| | | | TCTCTTACTCGTCATACCAAGATTCACTGA |
| SEQ ID | Amino | pb42 DLR amino | MDYKDHDGDYKDHDIDYKDDDDKMAPKKKRKVG |
| NO. 68 | Acid | acid sequence | IHGVPAAMAERPFQCRICMRNESRSSALTRHIR |
| | | | THTGEKPFACDICGRKFARSDTLTRHTKIHTGS |
| | | | QKPFQCRICMRNFSDRSNLTRHIRTHTGEKPFA |
| | | | CDICGRKFARSDNLTRHTKIHTGSQKPFQCRIC |
| | | | MRNFSRSDHLTRHIRTHTGLRGSGGGGGSGGGG |
| | | | GSGGGGGSGGGGGSGGGGGSGGGGGSMAERPFQ |
| | | | CRICMRNFSDRSNLTRHIRTHTGEKPFACDICG |
| | | | RKFARSDHLTRHTKIHTGSQKPFQCRICMRNES |
| | | | DRSNLTRHIRTHTGEKPFACDICGRKFARSDSL |
| | | | SEHTKIHTGSQKPFQCRICMRNESRSSNLTRHI |
| | | | RTHTGEKPFACDICGRKFARSDSLTRHTKIH* |
| SEQ ID | Amino | longer linker for | GGGGGSGGGGGSGGGGGSGGGGGSGGGGGSGGG |
| NO. 69 | Acid | pb42 | GGS |
| SEQ ID | DNA | donor template, | GTGGCATCGCCCTCGCCCTCGCCGGACACGCTG |
| NO. 70 | | 142bp | AACTTGTGGCCGTTTACGTCCCCGTCCAGCTCC |
| | | | ACGAGGATGGGGACGACGCCGGTGAACAGCTCC |
| | | | TCGCCCTTGCTCACCATAAGCTTAAGTTTAAAC |
| | | | GCTAGCCAGC |
| SEQ ID | DNA | pb35, plasmid full | GACTCTTCGCGATGTACGGGCCAGATATACGCG |
| NO. 71 | | length DNA | TTGACATTGATTATTGACTAGTTATTAATAGTA |
| | | sequence | ATCAATTACGGGGTCATTAGTTCATAGCCCATA |
| | | | TATGGAGTTCCGCGTTACATAACTTACGGTAAA |
| | | | TGGCCCGCCTGGCTGACCGCCCAACGACCCCCG |
| | | | CCCATTGACGTCAATAATGACGTATGTTCCCAT |
| | | | AGTAACGCCAATAGGGACTTTCCATTGACGTCA |
| | | | ATGGGTGGACTATTTACGGTAAACTGCCCACTT |
| | | | GGCAGTACATCAAGTGTATCATATGCCAAGTAC |
| | | | GCCCCCTATTGACGTCAATGACGGTAAATGGCC |
| | | | CGCCTGGCATTATGCCCAGTACATGACCTTATG |
| | | | GGACTTTCCTACTTGGCAGTACATCTACGTATT |
| | | | AGTCATCGCTATTACCATGGTGATGCGGTTTTG |
| | | | GCAGTACATCAATGGGCGTGGATAGCGGTTTGA |
| | | | CTCACGGGGATTTCCAAGTCTCCACCCCATTGA |
| | | | CGTCAATGGGAGTTTGTTTTGGCACCAAAATCA |
| | | | ACGGGACTTTCCAAAATGTCGTAACAACTCCGC |
| | | | CCCATTGACGCAAATGGGCGGTAGGCGTGTACG |
| | | | GTGGGAGGTCTATATAAGCAGAGCTCTCTGGCT |
| | | | AACTAGAGAACCCACTGCTTACTGGCTTATCGA |
| | | | AATTAATACGACTCACTATAGGGAGACCCAAGC |
| | | | TGGCTAGCGTTTAAACTTAAGCTTATGGACTAC |
| | | | AAAGACCATGACGGTGATTATAAAGATCATGAC |
| | | | ATCGATTACAAGGATGACGATGACAAGATGGCC |
| | | | CCCAAGAAGAAGAGGAAGGTGGGCATTCACGGG |
| | | | GTACCGGCGGCGATGGCCGAGCGGCCCTTCCAG |
| | | | TGCAGGATCTGTATGCGCAACTTTTCTCGTTCT |
| | | | TCTGCTCTTACTCGTCACATCAGAACCCATACA |
| | | | GGCGAAAAGCCTTTCGCCTGCGACATTTGTGGG |
| | | | AGAAAATTTGCTCGTTCTGATACTCTTACTCGT |
| | | | CATACCAAGATCCACACCGGCTCTCAGAAACCA |
| | | | TTCCAGTGCCGCATTTGTATGCGGAATTTTTCC |
| | | | GATCGTTCTAATCTTACTCGTCATATCCGCACT |
| | | | CACACCGGAGAGAAGCCCTTTGCTTGCGACATT |
| | | | TGTGGCAGGAAATTTGCTCGTTCTGATAATCTT |
| | | | ACTCGTCACACTAAGATCCATACTGGGTCACAG |
| | | | AAACCTTTCCAGTGCCGGATTTGTATGAGAAAC |
| | | | TTTAGCCGTTCTGATCATCTTACTCGTCACATC |
| | | | AGAACACATACTGGGCTGAGAGGATCCAATTCT |
| | | | GGTGATCCTCGGAGACACAGTCTGGGCGGTTCT |
| | | | CGTAAACCCGCTCTGATTGCCTATAAAAACTTT |
| | | | GATCTGCTGGTCATTGAACTTAAGCCTTGAGCG |
| | | | GCCGCTCGAGTCTAGAGGGCCCGTTTAAACCCG |
| | | | CTGATCAGCCTCGACTGTGCCTTCTAGTTGCCA |
| | | | GCCATCTGTTGTTTGCCCCTCCCCCGTGCCTTC |
| | | | CTTGACCCTGGAAGGTGCCACTCCCACTGTCCT |
| | | | TTCCTAATAAAATGAGGAAATTGCATCGCATTG |
| | | | TCTGAGTAGGTGTCATTCTATTCTGGGGGGTGG |
| | | | GGTGGGGCAGGACAGCAAGGGGGAGGATTGGGA |
| | | | AGACAATAGCAGGCATGCTGGGGATGCGGTGGG |
| | | | CTCTATGGCTTCTACTGGGCGGTTTTATGGACA |
| | | | GCAAGCGAACCGGAATTGCCAGCTGGGGCGCCC |
| | | | TCTGGTAAGGTTGGGAAGCCCTGCAAAGTAAAC |
| | | | TGGATGGCTTTCTCGCCGCCAAGGATCTGATGG |
| | | | CGCAGGGGATCAAGCTCTGATCAAGAGACAGGA |
| | | | TGAGGATCGTTTCGCATGATTGAACAAGATGGA |
| | | | TTGCACGCAGGTTCTCCGGCCGCTTGGGTGGAG |
| | | | AGGCTATTCGGCTATGACTGGGCACAACAGACA |
| | | | ATCGGCTGCTCTGATGCCGCCGTGTTCCGGCTG |
| | | | TCAGCGCAGGGGCGCCCGGTTCTTTTTGTCAAG |
| | | | ACCGACCTGTCCGGTGCCCTGAATGAACTGCAA |
| | | | GACGAGGCAGCGCGGCTATCGTGGCTGGCCACG |
| | | | ACGGGCGTTCCTTGCGCAGCTGTGCTCGACGTT |
| | | | GTCACTGAAGCGGGAAGGGACTGGCTGCTATTG |
| | | | GGCGAAGTGCCGGGGCAGGATCTCCTGTCATCT |
| | | | CACCTTGCTCCTGCCGAGAAAGTATCCATCATG |
| | | | GCTGATGCAATGCGGCGGCTGCATACGCTTGAT |
| | | | CCGGCTACCTGCCCATTCGACCACCAAGCGAAA |
| | | | CATCGCATCGAGCGAGCACGTACTCGGATGGAA |
| | | | GCCGGTCTTGTCGATCAGGATGATCTGGACGAA |
| | | | GAGCATCAGGGGCTCGCGCCAGCCGAACTGTTC |
| | | | GCCAGGCTCAAGGCGAGCATGCCCGACGGCGAG |
| | | | GATCTCGTCGTGACCCATGGCGATGCCTGCTTG |
| | | | CCGAATATCATGGTGGAAAATGGCCGCTTTTCT |
| | | | GGATTCATCGACTGTGGCCGGCTGGGTGTGGCG |
| | | | GACCGCTATCAGGACATAGCGTTGGCTACCCGT |
| | | | GATATTGCTGAAGAGCTTGGCGGCGAATGGGCT |
| | | | GACCGCTTCCTCGTGCTTTACGGTATCGCCGCT |
| | | | CCCGATTCGCAGCGCATCGCCTTCTATCGCCTT |
| | | | CTTGACGAGTTCTTCTGAATTATTAACGCTTAC |
| | | | AATTTCCTGATGCGGTATTTTCTCCTTACGCAT |
| | | | CTGTGCGGTATTTCACACCGCATACAGGTGGCA |
| | | | CTTTTCGGGGAAATGTGCGCGGAACCCCTATTT |
| | | | GTTTATTTTTCTAAATACATTCAAATATGTATC |
| | | | CGCTCATGAGACAATAACCCTGATAAATGCTTC |
| | | | AATAATAGCACGTGCTAAAACTTCATTTTTAAT |
| | | | TTAAAAGGATCTAGGTGAAGATCCTTTTTGATA |
| | | | ATCTCATGACCAAAATCCCTTAACGTGAGTTTT |
| | | | CGTTCCACTGAGCGTCAGACCCCGTAGAAAAGA |
| | | | TCAAAGGATCTTCTTGAGATCCTTTTTTTCTGC |
| | | | GCGTAATCTGCTGCTTGCAAACAAAAAAACCAC |
| | | | CGCTACCAGCGGTGGTTTGTTTGCCGGATCAAG |
| | | | AGCTACCAACTCTTTTTCCGAAGGTAACTGGCT |
| | | | TCAGCAGAGCGCAGATACCAAATACTGTCCTTC |
| | | | TAGTGTAGCCGTAGTTAGGCCACCACTTCAAGA |
| | | | ACTCTGTAGCACCGCCTACATACCTCGCTCTGC |
| | | | TAATCCTGTTACCAGTGGCTGCTGCCAGTGGCG |
| | | | ATAAGTCGTGTCTTACCGGGTTGGACTCAAGAC |
| | | | GATAGTTACCGGATAAGGCGCAGCGGTCGGGCT |
| | | | GAACGGGGGGTTCGTGCACACAGCCCAGCTTGG |
| | | | AGCGAACGACCTACACCGAACTGAGATACCTAC |
| | | | AGCGTGAGCTATGAGAAAGCGCCACGCTTCCCG |
| | | | AAGGGAGAAAGGCGGACAGGTATCCGGTAAGCG |
| | | | GCAGGGTCGGAACAGGAGAGCGCACGAGGGAGC |
| | | | TTCCAGGGGGAAACGCCTGGTATCTTTATAGTC |
| | | | CTGTCGGGTTTCGCCACCTCTGACTTGAGCGTC |
| | | | GATTTTTGTGATGCTCGTCAGGGGGGCGGAGCC |
| | | | TATGGAAAAACGCCAGCAACGCGGCCTTTTTAC |
| | | | GGTTCCTGGGCTTTTGCTGGCCTTTTGCTCACA |
| | | | TGTTCTT |
| SEQ ID | DNA | pb35, cDNA | ATGGACTACAAAGACCATGACGGTGATTATAAA |
| NO. 72 | | | GATCATGACATCGATTACAAGGATGACGATGAC |
| | | | AAGATGGCCCCCAAGAAGAAGAGGAAGGTGGGC |
| | | | ATTCACGGGGTACCGGCGGCGATGGCCGAGCGG |
| | | | CCCTTCCAGTGCAGGATCTGTATGCGCAACTTT |
| | | | TCTCGTTCTTCTGCTCTTACTCGTCACATCAGA |
| | | | ACCCATACAGGCGAAAAGCCTTTCGCCTGCGAC |
| | | | ATTTGTGGGAGAAAATTTGCTCGTTCTGATACT |
| | | | CTTACTCGTCATACCAAGATCCACACCGGCTCT |
| | | | CAGAAACCATTCCAGTGCCGCATTTGTATGCGG |
| | | | AATTTTTCCGATCGTTCTAATCTTACTCGTCAT |
| | | | ATCCGCACTCACACCGGAGAGAAGCCCTTTGCT |
| | | | TGCGACATTTGTGGCAGGAAATTTGCTCGTTCT |
| | | | GATAATCTTACTCGTCACACTAAGATCCATACT |
| | | | GGGTCACAGAAACCTTTCCAGTGCCGGATTTGT |
| | | | ATGAGAAACTTTAGCCGTTCTGATCATCTTACT |
| | | | CGTCACATCAGAACACATACTGGGCTGAGAGGA |
| | | | TCCAATTCTGGTGATCCTCGGAGACACAGTCTG |
| | | | GGCGGTTCTCGTAAACCCGCTCTGATTGCCTAT |
| | | | AAAAACTTTGATCTGCTGGTCATTGAACTTAAG |
| | | | CCTTGA |
| SEQ ID | aa | pb35, DLR amino | MDYKDHDGDYKDHDIDYKDDDDKMAPKKKRKVG |
| NO. 73 | | acid sequence | IHGVPAAMAERPFQCRICMRNFSRSSALTRHIR |
| | | | THTGEKPFACDICGRKFARSDTLTRHTKIHTGS |
| | | | QKPFQCRICMRNFSDRSNLTRHIRTHTGEKPFA |
| | | | CDICGRKFARSDNLTRHTKIHTGSQKPFQCRIC |
| | | | MRNFSRSDHLTRHIRTHTGLRGSNSGDPRRHSL |
| | | | GGSRKPALIAYKNFDLLVIELKP* |
| SEQ ID | DNA | pb34, cDNA | ATGGACTACAAAGACCATGACGGTGATTATAAA |
| NO. 74 | | | GATCATGACATCGATTACAAGGATGACGATGAC |
| | | | AAGATGGCCCCCAAGAAGAAGAGGAAGGTGGGC |
| | | | ATTCACGGGGTACCGGCGGCGATGGCCGAGCGG |
| | | | CCCTTCCAGTGCAGGATCTGTATGCGCAACTTT |
| | | | TCTCGTTCTTCTGCTCTTACTCGTCACATCAGA |
| | | | ACCCATACAGGCGAAAAGCCTTTCGCCTGCGAC |
| | | | ATTTGTGGGAGAAAATTTGCTCGTTCTGATACT |
| | | | CTTACTCGTCATACCAAGATCCACACCGGCTCT |
| | | | CAGAAACCATTCCAGTGCCGCATTTGTATGCGG |
| | | | AATTTTTCCGATCGTTCTAATCTTACTCGTCAT |
| | | | ATCCGCACTCACACCGGAGAGAAGCCCTTTGCT |
| | | | TGCGACATTTGTGGCAGGAAATTTGCTCGTTCT |
| | | | GATAATCTTACTCGTCACACTAAGATCCATACT |
| | | | GGGTCACAGAAACCTTTCCAGTGCCGGATTTGT |
| | | | ATGAGAAACTTTAGCCGTTCTGATCATCTTACT |
| | | | CGTCACATCAGAACACATACTGGGCTGAGAGGA |
| | | | TCCAATTCTGGTGATCCTCGGAGACACAGTCTG |
| | | | GGCGGTTCTCGTAAACCCGATCTGATTGCCTAT |
| | | | AAAAACTTTGATCTGCTGGTCATTGTTCTTAAG |
| | | | CCTTGA |
| SEQ ID | Amino | pb34, DLR amino | MDYKDHDGDYKDHDIDYKDDDDKMAPKKKRKVG |
| NO. 75 | Acid | acid sequence | IHGVPAAMAERPFQCRICMRNFSRSSALTRHIR |
| | | | THTGEKPFACDICGRKFARSDTLTRHTKIHTGS |
| | | | QKPFQCRICMRNFSDRSNLTRHIRTHTGEKPFA |
| | | | CDICGRKFARSDNLTRHTKIHTGSQKPFQCRIC |
| | | | MRNFSRSDHLTRHIRTHTGLRGSNSGDPRRHSL |
| | | | GGSRKPDLIAYKNFDLLVIVLKP* |
| SEQ ID | DNA | POP29 EGFPDP2 | CCATATATGGAGTTCCGCGTTAC |
| NO. 76 | | Sequencing forward | |
| | | primer | |
| SEQ ID | DNA | POP32 EGFPDP2 | GCTTGTCGGCCATGATATAG |
| NO. 77 | | Sequencing reverse | |
| | | primer | |
| SEQ ID | DNA | POP43 EGFPDP2- | CCAAGCTGGCTAGCGTTTA |
| NO. 78 | | 171 forward primer | |
| SEQ ID | DNA | POP44 EGFPDP2- | GAACTTCAGGGTCAGCTTGC |
| NO. 79 | | 171 reverse primer | |
| SEQ ID | DNA | POP37 112 R | GGTCATCGGCATCGCGGAGGAG |
| NO. 80 | | reverse primer | |
| SEQ ID | Amino | R-CORE AA | NSGDPRRHSLGGSRKPDLIAYKNFDLLVIELKP |
| NO. 81 | Acid | | |
| SEQ ID | DNA | 530 primer | CTCCGCGATGCCGATG |
| NO. 82 | | | |
| SEQ ID | DNA | 531 primer | CGCGGCCCTGTTCCA |
| NO. 83 | | | |
| SEQ ID | Amino | R domain of | NSGDPRRHSLGGSRKPALIAYKNFDLLVIELKP |
| NO. 84 | Acid | EGFPDP2 DLR, | |
| | | encoded in plasmid | |
| | | pb35 | |
| SEQ ID | DNA | R domain coding | AATTCTGGTGATCCTCGGAGACACAGTCTGGGC |
| NO. 85 | | sequence of plasmid | GGTTCTCGTAAACCCGCTCTGATTGCCTATAAA |
| | | pb35 | AACTTTGATCTGCTGGTCATTGAACTTAAGCCT |
| | | | TGA |
| SEQ ID | Amino | 6-zinc-finger array | MAERPFQCRICMRNFSDRSNLTRHIRTHTGEKP |
| NO. 86 | Acid | in R element | FACDICGRKFARSDHLTRHTKIHTGSQKPFQCR |
| | | | ICMRNFSDRSNLTRHIRTHTGEKPFACDICGRK |
| | | | FARSDSLSEHTKIHTGSQKPFQCRICMRNESRS |
| | | | SNLTRHIRTHTGEKPFACDICGRKFARSDSLTR |
| | | | HTKIH |
| SEQ ID | DNA | pb6 cDNA | ATGGCGGCGATGGCCGAGCGGCCCTTCCAGTGC |
| NO. 87 | | | AGGATCTGTATGCGCAACTTTTCTCGGTCCTCC |
| | | | GACCTGACCCGGCACATCAGAACCCATACAGGC |
| | | | GAAAAGCCTTTCGCCTGCGACATTTGTGGGAGA |
| | | | AAATTTGCTCGGTCCGACACCCTGACCCGGCAT |
| | | | ACCAAGATCCACACCGGCTCTCAGAAACCATTC |
| | | | CAGTGCCGCATTTGTATGCGGAATTTTTCCCAG |
| | | | TCCGGCGACCTGTCCGAGCATATCCGCACTCAC |
| | | | ACCGGAGAGAAGCCCTTTGCTTGCGACATTTGT |
| | | | GGCAGGAAATTTGCTACCTCCGGCCACCTGACC |
| | | | ACCCACACTAAGATCCATACTGGGTCACAGAAA |
| | | | CCTTTCCAGTGCCGGATTTGTATGAGAAACTTT |
| | | | AGCGACTCCTCCCACCTGACCACCCACATCAGA |
| | | | ACCCATACAGGCGAAAAGCCTTTCGCCTGCGAC |
| | | | ATTTGTGGGAGAAAATTTGCTCGGTCCTCCCAC |
| | | | CTGACCACCCATACCAAGATCCACACCGGCTCT |
| | | | CAGAAACCATTCCAGTGCCGCATTTGTATGCGG |
| | | | AATTTTTCCGACCGGTCCGACCTGACCCGGCAT |
| | | | ATCCGCACTCACACCGGAGAGAAGCCCTTTGCT |
| | | | TGCGACATTTGTGGCAGGAAATTTGCTGACCGG |
| | | | TCCGACCTGACCCGGCACACTAAGATCCATACT |
| | | | GGGTCACAGAAACCTTTCCAGTGCCGGATTTGT |
| | | | ATGAGAAACTTTAGCCGGTCCGACACCCTGACC |
| | | | CGGCACATCAGAACACATACTGGGCTGAGAGGA |
| | | | TCCAATTCTGGTGATCCTCGGAGACACAGTCTG |
| | | | GGCGGTTCTCGTAAACCCGATCTGATTGCCTAT |
| | | | AAAAACTTTGATCTGCTGGTCATTGTTCTTAAG |
| | | | CCTTGA |
| SEQ ID | Amino | pb6 DLR amino | MAAMAERPFQCRICMRNFSRSSDLTRHIRTHTG |
| NO. 88 | Acid | acid sequence | EKPFACDICGRKFARSDTLTRHTKIHTGSQKPF |
| | | | QCRICMRNFSQSGDLSEHIRTHTGEKPFACDIC |
| | | | GRKFATSGHLTTHTKIHTGSQKPFQCRICMRNF |
| | | | SDSSHLTTHIRTHTGEKPFACDICGRKFARSSH |
| | | | LTTHTKIHTGSQKPFQCRICMRNFSDRSDLTRH |
| | | | IRTHTGEKPFACDICGRKFADRSDLTRHTKIHT |
| | | | GSQKPFQCRICMRNFSRSDTLTRHIRTHTGLRG |
| | | | SNSGDPRRHSLGGSRKPDLIAYKNFDLLVIVLK |
| | | | P* |
| SEQ ID | DNA | pb41 cDNA | ATGGCGGCGATGGCCGAGCGGCCCTTCCAGTGC |
| NO. 89 | | | AGGATCTGTATGCGCAACTTTTCTGACCGGTCC |
| | | | CACCTGACCCGGCACATCAGAACCCATACAGGC |
| | | | GAAAAGCCTTTCGCCTGCGACATTTGTGGGAGA |
| | | | AAATTTGCTCGGTCCGACAACCTGACCCGGCAT |
| | | | ACCAAGATCCACACCGGCTCTCAGAAACCATTC |
| | | | CAGTGCCGCATTTGTATGCGGAATTTTTCCGAC |
| | | | TCCTCCCACCTGTCCGAGCATATCCGCACTCAC |
| | | | ACCGGAGAGAAGCCCTTTGCTTGCGACATTTGT |
| | | | GGCAGGAAATTTGCTGACCGGTCCGACCTGACC |
| | | | CGGCACACTAAGATCCATACTGGGTCACAGAAA |
| | | | CCTTTCCAGTGCCGGATTTGTATGAGAAACTTT |
| | | | AGCCGGTCCGACCACCTGACCCGGCACATCAGA |
| | | | ACCCATACAGGCGAAAAGCCTTTCGCCTGCGAC |
| | | | ATTTGTGGGAGAAAATTTGCTGACCGGTCCGAC |
| | | | CTGACCCGGCATACCAAGATCCACACCGGCTCT |
| | | | CAGAAACCATTCCAGTGCCGCATTTGTATGCGG |
| | | | AATTTTTCCCGGTCCGACAACCTGTCCGAGCAT |
| | | | ATCCGCACTCACACCGGAGAGAAGCCCTTTGCT |
| | | | TGCGACATTTGTGGCAGGAAATTTGCTGAGTCC |
| | | | TCCAACCTGACCACCCATACCAAGATCCACACC |
| | | | GGCTCTCAGAAACCATTCCAGTGCCGCATTTGT |
| | | | ATGCGGAATTTTTCCCGGTCCTCCTCCCTGACC |
| | | | CGGCATATCCGCACTCACACCGGAGAGAAGCCC |
| | | | TTTGCTTGCGACATTTGTGGCAGGAAATTTGCT |
| | | | CAGTCCTCCGACCTGACCCGGCACACTAAGATC |
| | | | CATACTGGGTCACAGAAACCTTTCCAGTGCCGG |
| | | | ATTTGTATGAGAAACTTTAGCCGGTCCGACTCC |
| | | | CTGTCCGAGCACATCAGAACACATACTGGGCTG |
| | | | AGAGGATCCAATTCTGGTGATCCTCGGAGACAC |
| | | | AGTCTGGGCGGTTCTCGTAAACCCGATCTGATT |
| | | | GCCTATAAAAACTTTGATCTGCTGGTCATTGTT |
| | | | CTTAAGCCTTGA |
| SEQ ID | Amino | p41 DLR amino | MAAMAERPFQCRICMRNFSDRSHLTRHIRTHTG |
| NO. 90 | Acid | acid sequence | EKPFACDICGRKFARSDNLTRHTKIHTGSQKPF |
| | | | QCRICMRNFSDSSHLSEHIRTHTGEKPFACDIC |
| | | | GRKFADRSDLTRHTKIHTGSQKPFQCRICMRNF |
| | | | SRSDHLTRHIRTHTGEKPFACDICGRKFADRSD |
| | | | LTRHTKIHTGSQKPFQCRICMRNFSRSDNLSEH |
| | | | IRTHTGEKPFACDICGRKFAESSNLTTHTKIHT |
| | | | GSQKPFQCRICMRNFSRSSSLTRHIRTHTGEKP |
| | | | FACDICGRKFAQSSDLTRHTKIHTGSQKPFQCR |
| | | | ICMRNFSRSDSLSEHIRTHTGLRGSNSGDPRRH |
| | | | SLGGSRKPDLIAYKNFDLLVIVLKP* |
| SEQ ID | DNA | Zinc finger frame 1 | TTCCAGTGCCGGATCTGCATGCGGAACTTCTCC |
| NO. 91 | | | NNNNNNNNNNNNNNNNNNNNNCACATCCGGACC |
| | | | CAC |
| SEQ ID | DNA | Zinc finger frame 2 | TTTGCGTGCGATATTTGCGGCCGTAAATTTGCG |
| NO. 92 | | | NNNNNNNNNNNNNNNNNNNNNCATACCAAAATT |
| | | | CAT |
| SEQ ID | DNA | EGFPDP2 DLR D | TTCCAGTGCAGGATCTGTATGCGCAACTTTTCT |
| NO. 93 | | element 5-zinc- | CGTTCTTCTGCTCTTACTCGTCACATCAGAACC |
| | | finger array | CATACAGGCGAAAAGCCTTTCGCCTGCGACATT |
| | | | TGTGGGAGAAAATTTGCTCGTTCTGATACTCTT |
| | | | ACTCGTCATACCAAGATCCACACCGGCTCTCAG |
| | | | AAACCATTCCAGTGCCGCATTTGTATGCGGAAT |
| | | | TTTTCCGATCGTTCTAATCTTACTCGTCATATC |
| | | | CGCACTCACACCGGAGAGAAGCCCTTTGCTTGC |
| | | | GACATTTGTGGCAGGAAATTTGCTCGTTCTGAT |
| | | | AATCTTACTCGTCACACTAAGATCCATACTGGG |
| | | | TCACAGAAACCTTTCCAGTGCCGGATTTGTATG |
| | | | AGAAACTTTAGCCGTTCTGATCATCTTACTCGT |
| | | | CACATCAGAACACATACTGGG |
| SEQ ID | DNA | EGFPDP2 DLR D | TTCCAGTGCAGGATCTGTATGCGCAACTTTTCC |
| NO. 94 | | element 6-zinc- | GATCGTTCTAATCTTACTCGTCACATCAGAACC |
| | | finger array | CATACAGGCGAAAAGCCTTTCGCCTGCGACATT |
| | | | TGTGGGCGGAAATTTGCTCGTTCTGATCATCTT |
| | | | ACTCGTCACACAAAGATCCATACTGGCAGCCAG |
| | | | AAACCATTCCAGTGCAGGATTTGCATGAGAAAC |
| | | | TTTTCCGATCGTTCTAATCTTACTCGTCACATC |
| | | | CGCACTCATACCGGAGAGAAGCCCTTTGCTTGC |
| | | | GACATTTGTGGCCGGAAATTTGCTCGTTCTGAT |
| | | | TCTCTTTCTGAACATACAAAGATCCATACTGGG |
| | | | TCTCAGAAACCTTTCCAGTGCAGGATTTGTATG |
| | | | AGAAATTTTTCCCGTTCTTCTAATCTTACTCGT |
| | | | CACATCAGAACACATACTGGGGAGAAGCCCTTT |
| | | | GCATGCGACATTTGTGGACGGAAATTTGCTCGT |
| | | | TCTGATTCTCTTACTCGTCATACCAAGATTCAC |
| SEQ ID | DNA | ApoE codon 112 | TTCCAGTGCAGGATCTGTATGCGCAACTTTTCT |
| NO. 95 | | site DLR D element | CGGTCCTCCGACCTGACCCGGCACATCAGAACC |
| | | 9-zinc-finger array | CATACAGGCGAAAAGCCTTTCGCCTGCGACATT |
| | | | TGTGGGAGAAAATTTGCTCGGTCCGACACCCTG |
| | | | ACCCGGCATACCAAGATCCACACCGGCTCTCAG |
| | | | AAACCATTCCAGTGCCGCATTTGTATGCGGAAT |
| | | | TTTTCCCAGTCCGGCGACCTGTCCGAGCATATC |
| | | | CGCACTCACACCGGAGAGAAGCCCTTTGCTTGC |
| | | | GACATTTGTGGCAGGAAATTTGCTACCTCCGGC |
| | | | CACCTGACCACCCACACTAAGATCCATACTGGG |
| | | | TCACAGAAACCTTTCCAGTGCCGGATTTGTATG |
| | | | AGAAACTTTAGCGACTCCTCCCACCTGACCACC |
| | | | CACATCAGAACCCATACAGGCGAAAAGCCTTTC |
| | | | GCCTGCGACATTTGTGGGAGAAAATTTGCTCGG |
| | | | TCCTCCCACCTGACCACCCATACCAAGATCCAC |
| | | | ACCGGCTCTCAGAAACCATTCCAGTGCCGCATT |
| | | | TGTATGCGGAATTTTTCCGACCGGTCCGACCTG |
| | | | ACCCGGCATATCCGCACTCACACCGGAGAGAAG |
| | | | CCCTTTGCTTGCGACATTTGTGGCAGGAAATTT |
| | | | GCTGACCGGTCCGACCTGACCCGGCACACTAAG |
| | | | ATCCATACTGGGTCACAGAAACCTTTCCAGTGC |
| | | | CGGATTTGTATGAGAAACTTTAGCCGGTCCGAC |
| | | | ACCCTGACCCGGCACATCAGAACACATACTGGG |
| SEQ ID | DNA | ApoE codon 158 | TTCCAGTGCAGGATCTGTATGCGCAACTTTTCT |
| NO. 96 | | site DLR D element | GACCGGTCCCACCTGACCCGGCACATCAGAACC |
| | | 11-zinc-finger array | CATACAGGCGAAAAGCCTTTCGCCTGCGACATT |
| | | | TGTGGGAGAAAATTTGCTCGGTCCGACAACCTG |
| | | | ACCCGGCATACCAAGATCCACACCGGCTCTCAG |
| | | | AAACCATTCCAGTGCCGCATTTGTATGCGGAAT |
| | | | TTTTCCGACTCCTCCCACCTGTCCGAGCATATC |
| | | | CGCACTCACACCGGAGAGAAGCCCTTTGCTTGC |
| | | | GACATTTGTGGCAGGAAATTTGCTGACCGGTCC |
| | | | GACCTGACCCGGCACACTAAGATCCATACTGGG |
| | | | TCACAGAAACCTTTCCAGTGCCGGATTTGTATG |
| | | | AGAAACTTTAGCCGGTCCGACCACCTGACCCGG |
| | | | CACATCAGAACCCATACAGGCGAAAAGCCTTTC |
| | | | GCCTGCGACATTTGTGGGAGAAAATTTGCTGAC |
| | | | CGGTCCGACCTGACCCGGCATACCAAGATCCAC |
| | | | ACCGGCTCTCAGAAACCATTCCAGTGCCGCATT |
| | | | TGTATGCGGAATTTTTCCCGGTCCGACAACCTG |
| | | | TCCGAGCATATCCGCACTCACACCGGAGAGAAG |
| | | | CCCTTTGCTTGCGACATTTGTGGCAGGAAATTT |
| | | | GCTGAGTCCTCCAACCTGACCACCCATACCAAG |
| | | | ATCCACACCGGCTCTCAGAAACCATTCCAGTGC |
| | | | CGCATTTGTATGCGGAATTTTTCCCGGTCCTCC |
| | | | TCCCTGACCCGGCATATCCGCACTCACACCGGA |
| | | | GAGAAGCCCTTTGCTTGCGACATTTGTGGCAGG |
| | | | AAATTTGCTCAGTCCTCCGACCTGACCCGGCAC |
| | | | ACTAAGATCCATACTGGGTCACAGAAACCTTTC |
| | | | CAGTGCCGGATTTGTATGAGAAACTTTAGCCGG |
| | | | TCCGACTCCCTGTCCGAGCACATCAGAACACAT |
| | | | ACTGGG |
| SEQ ID | DNA | dCas9 | GACAAGAAGTACAGCATCGGCCTGGCCATCGGC |
| NO. 97 | | | ACCAACTCTGTGGGCTGGGCCGTGATCACCGAC |
| | | | GAGTACAAGGTGCCCAGCAAGAAATTCAAGGTG |
| | | | CTGGGCAACACCGACCGGCACAGCATCAAGAAG |
| | | | AACCTGATCGGAGCCCTGCTGTTCGACAGCGGC |
| | | | GAAACAGCCGAGGCCACCCGGCTGAAGAGAACC |
| | | | GCCAGAAGAAGATACACCAGACGGAAGAACCGG |
| | | | ATCTGCTATCTGCAAGAGATCTTCAGCAACGAG |
| | | | ATGGCCAAGGTGGACGACAGCTTCTTCCACAGA |
| | | | CTGGAAGAGTCCTTCCTGGTGGAAGAGGATAAG |
| | | | AAGCACGAGCGGCACCCCATCTTCGGCAACATC |
| | | | GTGGACGAGGTGGCCTACCACGAGAAGTACCCC |
| | | | ACCATCTACCACCTGAGAAAGAAACTGGTGGAC |
| | | | AGCACCGACAAGGCCGACCTGCGGCTGATCTAT |
| | | | CTGGCCCTGGCCCACATGATCAAGTTCCGGGGC |
| | | | CACTTCCTGATCGAGGGCGACCTGAACCCCGAC |
| | | | AACAGCGACGTGGACAAGCTGTTCATCCAGCTG |
| | | | GTGCAGACCTACAACCAGCTGTTCGAGGAAAAC |
| | | | CCCATCAACGCCAGCGGCGTGGACGCCAAGGCC |
| | | | ATCCTGTCTGCCAGACTGAGCAAGAGCAGACGG |
| | | | CTGGAAAATCTGATCGCCCAGCTGCCCGGCGAG |
| | | | AAGAAGAATGGCCTGTTCGGCAACCTGATTGCC |
| | | | CTGAGCCTGGGCCTGACCCCCAACTTCAAGAGC |
| | | | AACTTCGACCTGGCCGAGGATGCCAAACTGCAG |
| | | | CTGAGCAAGGACACCTACGACGACGACCTGGAC |
| | | | AACCTGCTGGCCCAGATCGGCGACCAGTACGCC |
| | | | GACCTGTTTCTGGCCGCCAAGAACCTGTCCGAC |
| | | | GCCATCCTGCTGAGCGACATCCTGAGAGTGAAC |
| | | | ACCGAGATCACCAAGGCCCCCCTGAGCGCCTCT |
| | | | ATGATCAAGAGATACGACGAGCACCACCAGGAC |
| | | | CTGACCCTGCTGAAAGCTCTCGTGCGGCAGCAG |
| | | | CTGCCTGAGAAGTACAAAGAGATTTTCTTCGAC |
| | | | CAGAGCAAGAACGGCTACGCCGGCTACATTGAC |
| | | | GGCGGAGCCAGCCAGGAAGAGTTCTACAAGTTC |
| | | | ATCAAGCCCATCCTGGAAAAGATGGACGGCACC |
| | | | GAGGAACTGCTCGTGAAGCTGAACAGAGAGGAC |
| | | | CTGCTGCGGAAGCAGCGGACCTTCGACAACGGC |
| | | | AGCATCCCCCACCAGATCCACCTGGGAGAGCTG |
| | | | CACGCCATTCTGCGGCGGCAGGAAGATTTTTAC |
| | | | CCATTCCTGAAGGACAACCGGGAAAAGATCGAG |
| | | | AAGATCCTGACCTTCCGCATCCCCTACTACGTG |
| | | | GGCCCTCTGGCCAGGGGAAACAGCAGATTCGCC |
| | | | TGGATGACCAGAAAGAGCGAGGAAACCATCACC |
| | | | CCCTGGAACTTCGAGGAAGTGGTGGACAAGGGC |
| | | | GCTTCCGCCCAGAGCTTCATCGAGCGGATGACC |
| | | | AACTTCGATAAGAACCTGCCCAACGAGAAGGTG |
| | | | CTGCCCAAGCACAGCCTGCTGTACGAGTACTTC |
| | | | ACCGTGTATAACGAGCTGACCAAAGTGAAATAC |
| | | | GTGACCGAGGGAATGAGAAAGCCCGCCTTCCTG |
| | | | AGCGGCGAGCAGAAAAAGGCCATCGTGGACCTG |
| | | | CTGTTCAAGACCAACCGGAAAGTGACCGTGAAG |
| | | | CAGCTGAAAGAGGACTACTTCAAGAAAATCGAG |
| | | | TGCTTCGACTCCGTGGAAATCTCCGGCGTGGAA |
| | | | GATCGGTTCAACGCCTCCCTGGGCACATACCAC |
| | | | GATCTGCTGAAAATTATCAAGGACAAGGACTTC |
| | | | CTGGACAATGAGGAAAACGAGGACATTCTGGAA |
| | | | GATATCGTGCTGACCCTGACACTGTTTGAGGAC |
| | | | AGAGAGATGATCGAGGAACGGCTGAAAACCTAT |
| | | | GCCCACCTGTTCGACGACAAAGTGATGAAGCAG |
| | | | CTGAAGCGGCGGAGATACACCGGCTGGGGCAGG |
| | | | CTGAGCCGGAAGCTGATCAACGGCATCCGGGAC |
| | | | AAGCAGTCCGGCAAGACAATCCTGGATTTCCTG |
| | | | AAGTCCGACGGCTTCGCCAACAGAAACTTCATG |
| | | | CAGCTGATCCACGACGACAGCCTGACCTTTAAA |
| | | | GAGGACATCCAGAAAGCCCAGGTGTCCGGCCAG |
| | | | GGCGATAGCCTGCACGAGCACATTGCCAATCTG |
| | | | GCCGGCAGCCCCGCCATTAAGAAGGGCATCCTG |
| | | | CAGACAGTGAAGGTGGTGGACGAGCTCGTGAAA |
| | | | GTGATGGGCCGGCACAAGCCCGAGAACATCGTG |
| | | | ATCGAAATGGCCAGAGAGAACCAGACCACCCAG |
| | | | AAGGGACAGAAGAACAGCCGCGAGAGAATGAAG |
| | | | CGGATCGAAGAGGGCATCAAAGAGCTGGGCAGC |
| | | | CAGATCCTGAAAGAACACCCCGTGGAAAACACC |
| | | | CAGCTGCAGAACGAGAAGCTGTACCTGTACTAC |
| | | | CTGCAGAATGGGCGGGATATGTACGTGGACCAG |
| | | | GAACTGGACATCAACCGGCTGTCCGACTACGAT |
| | | | GTGGACGCCATCGTGCCTCAGAGCTTTCTGAAG |
| | | | GACGACTCCATCGACAACAAGGTGCTGACCAGA |
| | | | AGCGACAAGAACCGGGGCAAGAGCGACAACGTG |
| | | | CCCTCCGAAGAGGTCGTGAAGAAGATGAAGAAC |
| | | | TACTGGCGGCAGCTGCTGAACGCCAAGCTGATT |
| | | | ACCCAGAGAAAGTTCGACAATCTGACCAAGGCC |
| | | | GAGAGAGGCGGCCTGAGCGAACTGGATAAGGCC |
| | | | GGCTTCATCAAGAGACAGCTGGTGGAAACCCGG |
| | | | CAGATCACAAAGCACGTGGCACAGATCCTGGAC |
| | | | TCCCGGATGAACACTAAGTACGACGAGAATGAC |
| | | | AAGCTGATCCGGGAAGTGAAAGTGATCACCCTG |
| | | | AAGTCCAAGCTGGTGTCCGATTTCCGGAAGGAT |
| | | | TTCCAGTTTTACAAAGTGCGCGAGATCAACAAC |
| | | | TACCACCACGCCCACGACGCCTACCTGAACGCC |
| | | | GTCGTGGGAACCGCCCTGATCAAAAAGTACCCT |
| | | | AAGCTGGAAAGCGAGTTCGTGTACGGCGACTAC |
| | | | AAGGTGTACGACGTGCGGAAGATGATCGCCAAG |
| | | | AGCGAGCAGGAAATCGGCAAGGCTACCGCCAAG |
| | | | TACTTCTTCTACAGCAACATCATGAACTTTTTC |
| | | | AAGACCGAGATTACCCTGGCCAACGGCGAGATC |
| | | | CGGAAGCGGCCTCTGATCGAGACAAACGGCGAA |
| | | | ACCGGGGAGATCGTGTGGGATAAGGGCCGGGAT |
| | | | TTTGCCACCGTGCGGAAAGTGCTGAGCATGCCC |
| | | | CAAGTGAATATCGTGAAAAAGACCGAGGTGCAG |
| | | | ACAGGCGGCTTCAGCAAAGAGTCTATCCTGCCC |
| | | | AAGAGGAACAGCGATAAGCTGATCGCCAGAAAG |
| | | | AAGGACTGGGACCCTAAGAAGTACGGCGGCTTC |
| | | | GACAGCCCCACCGTGGCCTATTCTGTGCTGGTG |
| | | | GTGGCCAAAGTGGAAAAGGGCAAGTCCAAGAAA |
| | | | CTGAAGAGTGTGAAAGAGCTGCTGGGGATCACC |
| | | | ATCATGGAAAGAAGCAGCTTCGAGAAGAATCCC |
| | | | ATCGACTTTCTGGAAGCCAAGGGCTACAAAGAA |
| | | | GTGAAAAAGGACCTGATCATCAAGCTGCCTAAG |
| | | | TACTCCCTGTTCGAGCTGGAAAACGGCCGGAAG |
| | | | AGAATGCTGGCCTCTGCCGGCGAACTGCAGAAG |
| | | | GGAAACGAACTGGCCCTGCCCTCCAAATATGTG |
| | | | AACTTCCTGTACCTGGCCAGCCACTATGAGAAG |
| | | | CTGAAGGGCTCCCCCGAGGATAATGAGCAGAAA |
| | | | CAGCTGTTTGTGGAACAGCACAAGCACTACCTG |
| | | | GACGAGATCATCGAGCAGATCAGCGAGTTCTCC |
| | | | AAGAGAGTGATCCTGGCCGACGCTAATCTGGAC |
| | | | AAAGTGCTGTCCGCCTACAACAAGCACCGGGAT |
| | | | AAGCCCATCAGAGAGCAGGCCGAGAATATCATC |
| | | | CACCTGTTTACCCTGACCAATCTGGGAGCCCCT |
| | | | GCCGCCTTCAAGTACTTTGACACCACCATCGAC |
| | | | CGGAAGAGGTACACCAGCACCAAAGAGGTGCTG |
| | | | GACGCCACCCTGATCCACCAGAGCATCACCGGC |
| | | | CTGTACGAGACACGGATCGACCTGTCTCAGCTG |
| | | | GGAGGCGAC |
| SEQ ID | DNA | Linker LRGS (SEQ | CTGAGAGGATCC |
| NO. 98 | | ID NO. 1) | |
| SEQ ID | DNA | Linker | CTGAGACAGAAGGACGCCGCCCGGGGATCC |
| NO. 99 | | LRQKDAARGS | |
| | | (SEQ ID NO. 13) | |
| SEQ ID | DNA | Linker | GGCGGCGGCGGCGGCTCCGGCGGCGGCGGCGGC |
| NO. 100 | | GGGGGSGGGGG | TCCGGCGGCGGCGGCGGCTCCGGCGGCGGCGGC |
| | | SGGGGGSGGGG | GGCTCCGGCGGCGGCGGCGGCTCCGGCGGCGGC |
| | | GSGGGGGSGGG | GGCGGCTCC |
| | | GGS (SEQ ID NO. | |
| | | 69) | |
| SEQ ID | Amino | R-core pb1 | NSGDPRRHSLGGSRKPDLIAYKNFDLLVIQLKP |
| NO. 101 | Acid | | |
| SEQ ID | Amino | R-core pb2 | NSGDPRRHSLGGSRKPDLIAYKNFDLLVINLKP |
| NO. 102 | Acid | | |
| SEQ ID | Amino | R-core pb3 | NSGDPRRHSLGGSRKPDLIAYKNFDLLVISLKP |
| NO. 103 | Acid | | |
| SEQ ID | Amino | R-core pb4 | NSGDPRRHSLGGSRKPDLIAYKNFDLLVITLKP |
| NO. 104 | Acid | | |
| SEQ ID | Amino | R-core pb5 | NSGDPRRHSLGGSRKPDLIAYKNFDLLVIALKP |
| NO. 105 | Acid | | |
| SEQ ID | Amino | R-core pb6 | NSGDPRRHSLGGSRKPDLIAYKNFDLLVIVLKP |
| NO. 106 | Acid | | |
| SEQ ID | Amino | R-core pb7 | NSGDPRRHSLGGSRKPDLIAYKNFDLLVILLKP |
| NO. 107 | Acid | | |
| SEQ ID | Amino | R-core pb8 | NSGDPRRHSLGGSRKPDLIAYKNFDLLVIILKP |
| NO. 108 | Acid | | |
| SEQ ID | Amino | R-core pb9 | NSGDPRRHSLGGSRKPDLIAYKNFDLLVIHLKP |
| NO. 109 | Acid | | |
| SEQ ID | Amino | R-core pb10 | NSGDPRRHSLGGSRKPDLIAYKNFDLLVIRLKP |
| NO. 110 | Acid | | |
| SEQ ID | Amino | R-core pb11 | NSGDPRRHSLGGSRKPDLIAYKNFDLLVIKLKP |
| NO. 111 | Acid | | |
| SEQ ID | Amino | R-core pb12 | NSGDPRRHSLGGSRKPDLIAYKNFDLLVIMLKP |
| NO. 112 | Acid | | |
| SEQ ID | Amino | R-core pb16 | NSGDPRRHSLGGSRKPNLIAYKNFDLLVIELKP |
| NO. 113 | Acid | | |
| SEQ ID | Amino | R-core pb17 | NSGDPRRHSLGGSRKPALIAYKNFDLLVIELKP |
| NO. 114 | Acid | | |
| SEQ ID | Amino | R-core pb18 | NSGDPRRHSLGGSRKPDGAIYTVGSPIDYGVIV |
| NO. 115 | Acid | | VTKP |
| SEQ ID | Amino | R-core pb19 | NSGDPRRHSLGGSRKPDFTLYKPSEPNKKIAIV |
| NO. 116 | Acid | | IKP |
| SEQ ID | Amino | R-core pb20 | NSGDPRRHSLGGSRKPDGLLWDDDCAIILVSKP |
| NO. 117 | Acid | | |
| SEQ ID | Amino | R-core pb21 | NSGDPRRHSLGGSRKPDHIYQLVYNSTDTLLLI |
| NO. 118 | Acid | | VSKP |
| SEQ ID | Amino | R-core pb22 | NSGDPRRHSLGGSRKPDHIYIFNDDNNTKNGLI |
| NO. 119 | Acid | | IVSKP |
| SEQ ID | Amino | R-core pb23 | NSGDPRRHSLGGSRKPDHVIQILDLFEKPLLLS |
| NO. 120 | Acid | | IVSKP |
| SEQ ID | Amino | R-core pb24 | NSGDPRRHSLGGSRKPDIILVNDNISLILILVA |
| NO. 121 | Acid | | KP |
| SEQ ID | Amino | R-core pb25 | NSGDPRRHSLGGSRKPDLIAYKNDDLLVIVAKP |
| NO. 122 | Acid | | |
| SEQ ID | Amino | R-core pb26 | NSGDPRRHSLGKIVPALIAYKNFDLLVIELKP |
| NO. 123 | Acid | | |
| SEQ ID | Amino | R-core pb27 | NSGDPRRHSLGGSNKPALIAYKNFDLLVIELKP |
| NO. 124 | Acid | | |
| SEQ ID | Amino | R-core pb28 | NSGDPRRHSLGTKRPALIAYKNFDLLVIELKP |
| NO. 125 | Acid | | |
| SEQ ID | Amino | R-core pb29 | NSGDPRRHSLGGETKRPALIAYKNFDLLVIELK |
| NO. 126 | Acid | | P |
| SEQ ID | Amino | R-core pb30 | NSGDPRRHSLGGKRPALIAYKNFDLLVIELKP |
| NO. 127 | Acid | | |
| SEQ ID | Amino | R-core pb31 | NSGDPRRHSLGREDERPALIAYKNEDLLVIELK |
| NO. 128 | Acid | | P |
| SEQ ID | DNA | R-core pb 1 | AATTCTGGTGATCCTCGGAGACACAGTCTGGGC |
| NO. 129 | | | GGTTCTCGTAAACCCGATCTGATTGCCTATAAA |
| | | | AACTTTGATCTGCTGGTCATTCAACTTAAGCCT |
| SEQ ID | DNA | R-core pb2 | AATTCTGGTGATCCTCGGAGACACAGTCTGGGC |
| NO. 130 | | | GGTTCTCGTAAACCCGATCTGATTGCCTATAAA |
| | | | AACTTTGATCTGCTGGTCATTAATCTTAAGCCT |
| SEQ ID | DNA | R-core pb3 | AATTCTGGTGATCCTCGGAGACACAGTCTGGGC |
| NO. 131 | | | GGTTCTCGTAAACCCGATCTGATTGCCTATAAA |
| | | | AACTTTGATCTGCTGGTCATTTCTCTTAAGCCT |
| SEQ ID | DNA | R-core pb4 | AATTCTGGTGATCCTCGGAGACACAGTCTGGGC |
| NO. 132 | | | GGTTCTCGTAAACCCGATCTGATTGCCTATAAA |
| | | | AACTTTGATCTGCTGGTCATTACTCTTAAGCCT |
| SEQ ID | DNA | R-core pb5 | AATTCTGGTGATCCTCGGAGACACAGTCTGGGC |
| NO. 133 | | | GGTTCTCGTAAACCCGATCTGATTGCCTATAAA |
| | | | AACTTTGATCTGCTGGTCATTGCTCTTAAGCCT |
| SEQ ID | DNA | R-core pb6 | AATTCTGGTGATCCTCGGAGACACAGTCTGGGC |
| NO. 134 | | | GGTTCTCGTAAACCCGATCTGATTGCCTATAAA |
| | | | AACTTTGATCTGCTGGTCATTGTTCTTAAGCCT |
| SEQ ID | DNA | R-core pb7 | AATTCTGGTGATCCTCGGAGACACAGTCTGGGC |
| NO. 135 | | | GGTTCTCGTAAACCCGATCTGATTGCCTATAAA |
| | | | AACTTTGATCTGCTGGTCATTCTTCTTAAGCCT |
| SEQ ID | DNA | R-core pb8 | AATTCTGGTGATCCTCGGAGACACAGTCTGGGC |
| NO. 136 | | | GGTTCTCGTAAACCCGATCTGATTGCCTATAAA |
| | | | AACTTTGATCTGCTGGTCATTATTCTTAAGCCT |
| SEQ ID | DNA | R-core pb9 | AATTCTGGTGATCCTCGGAGACACAGTCTGGGC |
| NO. 137 | | | GGTTCTCGTAAACCCGATCTGATTGCCTATAAA |
| | | | AACTTTGATCTGCTGGTCATTCATCTTAAGCCT |
| SEQ ID | DNA | R-core pb 10 | AATTCTGGTGATCCTCGGAGACACAGTCTGGGC |
| NO. 138 | | | GGTTCTCGTAAACCCGATCTGATTGCCTATAAA |
| | | | AACTTTGATCTGCTGGTCATTCGTCTTAAGCCT |
| SEQ ID | DNA | R-core pb11 | AATTCTGGTGATCCTCGGAGACACAGTCTGGGC |
| NO. 139 | | | GGTTCTCGTAAACCCGATCTGATTGCCTATAAA |
| | | | AACTTTGATCTGCTGGTCATTAAACTTAAGCCT |
| SEQ ID | DNA | R-core pb 12 | AATTCTGGTGATCCTCGGAGACACAGTCTGGGC |
| NO. 140 | | | GGTTCTCGTAAACCCGATCTGATTGCCTATAAA |
| | | | AACTTTGATCTGCTGGTCATTATGCTTAAGCCT |
| SEQ ID | DNA | R-core pb 16 | AATTCTGGTGATCCTCGGAGACACAGTCTGGGC |
| NO. 141 | | | GGTTCTCGTAAACCCAATCTGATTGCCTATAAA |
| | | | AACTTTGATCTGCTGGTCATTGAACTTAAGCCT |
| SEQ ID | DNA | R-core pb 17 | AATTCTGGTGATCCTCGGAGACACAGTCTGGGC |
| NO. 142 | | | GGTTCTCGTAAACCCGCTCTGATTGCCTATAAA |
| | | | AACTTTGATCTGCTGGTCATTGAACTTAAGCCT |
| SEQ ID | DNA | R-core pb18 | AATTCTGGTGATCCTCGGAGACACAGTCTGGGC |
| NO. 143 | | | GGTTCTCGTAAACCCGATGGTGCTATTTATACT |
| | | | GTTGGTTCTCCTATTGATTATGGTGTTATTGTT |
| | | | GTTACTAAACCT |
| SEQ ID | DNA | R-core pb 19 | AATTCTGGTGATCCTCGGAGACACAGTCTGGGC |
| NO. 144 | | | GGTTCTCGTAAACCCGATTTTACTCTTTATAAA |
| | | | CCTTCTGAACCTAATAAAAAAATTGCTATTGTT |
| | | | ATTAAACCT |
| SEQ ID | DNA | R-core pb20 | AATTCTGGTGATCCTCGGAGACACAGTCTGGGC |
| NO. 145 | | | GGTTCTCGTAAACCCGATGGTCTTCTTTGGGAT |
| | | | GATGATTGTGCTATTATTCTTGTTTCTAAACCT |
| SEQ ID | DNA | R-core pb21 | AATTCTGGTGATCCTCGGAGACACAGTCTGGGC |
| NO. 146 | | | GGTTCTCGTAAACCCGATCATATTTATCAACTT |
| | | | GTTTATAATTCTACTGATACTCTTCTTCTTATT |
| | | | GTTTCTAAACCT |
| SEQ ID | DNA | R-core pb22 | AATTCTGGTGATCCTCGGAGACACAGTCTGGGC |
| NO. 147 | | | GGTTCTCGTAAACCCGATCATATTTATATTTTT |
| | | | AATGATGATAATAATACTAAAAATGGTCTTATT |
| | | | ATTGTTTCTAAACCT |
| SEQ ID | DNA | R-core pb23 | AATTCTGGTGATCCTCGGAGACACAGTCTGGGC |
| NO. 148 | | | GGTTCTCGTAAACCCGATCATGTTATTCAAATT |
| | | | CTTGATCTTTTTGAAAAACCTCTTCTTCTTTCT |
| | | | ATTGTTTCTAAACCT |
| SEQ ID | DNA | R-core pb24 | AATTCTGGTGATCCTCGGAGACACAGTCTGGGC |
| NO. 149 | | | GGTTCTCGTAAACCCGATATTATTCTTGTTAAT |
| | | | GATAATATTTCTCTTATTCTTATTCTTGTTGCT |
| | | | AAACCT |
| SEQ ID | DNA | R-core pb25 | AATTCTGGTGATCCTCGGAGACACAGTCTGGGC |
| NO. 150 | | | GGTTCTCGTAAACCCGATCTTATTGCTTATAAA |
| | | | AATGATGATCTTCTTGTTATTGTTGCTAAACCT |
| SEQ ID | DNA | R-core pb26 | AATTCTGGTGATCCTCGGAGACACAGTCTGGGC |
| NO. 151 | | | AAGATCGTGCCCGCTCTGATTGCCTATAAAAAC |
| | | | TTTGATCTGCTGGTCATTGAACTTAAGCCT |
| SEQ ID | DNA | R-core pb27 | AATTCTGGTGATCCTCGGAGACACAGTCTGGGC |
| NO. 152 | | | GGTTCTAACAAACCCGCTCTGATTGCCTATAAA |
| | | | AACTTTGATCTGCTGGTCATTGAACTTAAGCCT |
| SEQ ID | DNA | R-core pb28 | AATTCTGGTGATCCTCGGAGACACAGTCTGGGC |
| NO. 153 | | | ACCAAGCGGCCCGCTCTGATTGCCTATAAAAAC |
| | | | TTTGATCTGCTGGTCATTGAACTTAAGCCT |
| SEQ ID | DNA | R-core pb29 | AATTCTGGTGATCCTCGGAGACACAGTCTGGGC |
| NO. 154 | | | GGTGAGACCAAGCGGCCCGCTCTGATTGCCTAT |
| | | | AAAAACTTTGATCTGCTGGTCATTGAACTTAAG |
| | | | CCT |
| SEQ ID | DNA | R-core pb30 | AATTCTGGTGATCCTCGGAGACACAGTCTGGGC |
| NO. 155 | | | GGTAAGCGGCCCGCTCTGATTGCCTATAAAAAC |
| | | | TTTGATCTGCTGGTCATTGAACTTAAGCCT |
| SEQ ID | DNA | R-core pb31 | AATTCTGGTGATCCTCGGAGACACAGTCTGGGC |
| NO. 156 | | | CGGGAGGACGAGCGGCCCGCTCTGATTGCCTAT |
| | | | AAAAACTTTGATCTGCTGGTCATTGAACTTAAG |
| | | | CCTTGA |
| SEQ ID | DNA | human ApoE gene | GGGACAGGGGGAGCCCTATAATTGGACAAGTCT |
| NO. 157 | | Sequence ID: | GGGATCCTTGAGTCCTACTCAGCCCCAGCGGAG |
| | | NG_007084.2 | GTGAAGGACGTCCTTCCCCAGGAGCCGGTGAGA |
| | | | AGCGCAGTCGGGGGCACGGGGATGAGCTCAGGG |
| | | | GCCTCTAGAAAGAGCTGGGACCCTGGGAACCCC |
| | | | TGGCCTCCAGGTAGTCTCAGGAGAGCTACTCGG |
| | | | GGTCGGGCTTGGGGAGAGGAGGAGCGGGGGTGA |
| | | | GGCAAGCAGCAGGGGACTGGACCTGGGAAGGGC |
| | | | TGGGCAGCAGAGACGACCCGACCCGCTAGAAGG |
| | | | TGGGGTGGGGAGAGCAGCTGGACTGGGATGTAA |
| | | | GCCATAGCAGGACTCCACGAGTTGTCACTATCA |
| | | | TTTATCGAGCACCTACTGGGTGTCCCCAGTGTC |
| | | | CTCAGATCTCCATAACTGGGGAGCCAGGGGCAG |
| | | | CGACACGGTAGCTAGCCGTCGATTGGAGAACTT |
| | | | TAAAATGAGGACTGAATTAGCTCATAAATGGAA |
| | | | CACGGCGCTTAACTGTGAGGTTGGAGCTTAGAA |
| | | | TGTGAAGGGAGAATGAGGAATGCGAGACTGGGA |
| | | | CTGAGATGGAACCGGCGGTGGGGAGGGGGTGGG |
| | | | GGGATGGAATTTGAACCCCGGGAGAGGAAGATG |
| | | | GAATTTTCTATGGAGGCCGACCTGGGGATGGGG |
| | | | AGATAAGAGAAGACCAGGAGGGAGTTAAATAGG |
| | | | GAATGGGTTGGGGGCGGCTTGGTAAATGTGCTG |
| | | | GGATTAGGCTGTTGCAGATAATGCAACAAGGCT |
| | | | TGGAAGGCTAACCTGGGGTGAGGCCGGGTTGGG |
| | | | GCCGGGCTGGGGGTGGGAGGAGTCCTCACTGGC |
| | | | GGTTGATTGACAGTTTCTCCTTCCCCAGACTGG |
| | | | CCAATCACAGGCAGGAAGATGAAGGTTCTGTGG |
| | | | GCTGCGTTGCTGGTCACATTCCTGGCAGGTATG |
| | | | GGGGCGGGGCTTGCTCGGTTCCCCCCGCTCCTC |
| | | | CCCCTCTCATCCTCACCTCAACCTCCTGGCCCC |
| | | | ATTCAGGCAGACCCTGGGCCCCCTCTTCTGAGG |
| | | | CTTCTGTGCTGCTTCCTGGCTCTGAACAGCGAT |
| | | | TTGACGCTCTCTGGGCCTCGGTTTCCCCCATCC |
| | | | TTGAGATAGGAGTTAGAAGTTGTTTTGTTGTTG |
| | | | TTGTTTGTTGTTGTTGTTTTGTTTTTTTGAGAT |
| | | | GAAGTCTCGCTCTGTCGCCCAGGCTGGAGTGCA |
| | | | GTGGCGGGATCTCGGCTCACTGCAAGCTCCGCC |
| | | | TCCCAGGTCCACGCCATTCTCCTGCCTCAGCCT |
| | | | CCCAAGTAGCTGGGACTACAGGCACATGCCACC |
| | | | ACACCCGACTAACTTTTTTGTATTTTCAGTAGA |
| | | | GACGGGGTTTCACCATGTTGGCCAGGCTGGTCT |
| | | | GGAACTCCTGACCTCAGGTGATCTGCCCGTTTC |
| | | | GATCTCCCAAAGTGCTGGGATTACAGGCGTGAG |
| | | | CCACCGCACCTGGCTGGGAGTTAGAGGTTTCTA |
| | | | ATGCATTGCAGGCAGATAGTGAATACCAGACAC |
| | | | GGGGCAGCTGTGATCTTTATTCTCCATCACCCC |
| | | | CACACAGCCCTGCCTGGGGCACACAAGGACACT |
| | | | CAATACATGCTTTTCCGCTGGGCGCGGTGGCTC |
| | | | ACCCCTGTAATCCCAGCACTTTGGGAGGCCAAG |
| | | | GTGGGAGGATCACTTGAGCCCAGGAGTTCAACA |
| | | | CCAGCCTGGGCAACATAGTGAGACCCTGTCTCT |
| | | | ACTAAAAATACAAAAATTAGCCAGGCATGGTGC |
| | | | CACACACCTGTGCTCTCAGCTACTCAGGAGGCT |
| | | | GAGGCAGGAGGATCGCTTGAGCCCAGAAGGTCA |
| | | | AGGTTGCAGTGAACCATGTTCAGGCCGCTGCAC |
| | | | TCCAGCCTGGGTGACAGAGCAAGACCCTGTTTA |
| | | | TAAATACATAATGCTTTCCAAGTGATTAAACCG |
| | | | ACTCCCCCCTCACCCTGCCCACCATGGCTCCAA |
| | | | AGAAGCATTTGTGGAGCACCTTCTGTGTGCCCC |
| | | | TAGGTACTAGATGCCTGGACGGGGTCAGAAGGA |
| | | | CCCTGACCCACCTTGAACTTGTTCCACACAGGA |
| | | | TGCCAGGCCAAGGTGGAGCAAGCGGTGGAGACA |
| | | | GAGCCGGAGCCCGAGCTGCGCCAGCAGACCGAG |
| | | | TGGCAGAGCGGCCAGCGCTGGGAACTGGCACTG |
| | | | GGTCGCTTTTGGGATTACCTGCGCTGGGTGCAG |
| | | | ACACTGTCTGAGCAGGTGCAGGAGGAGCTGCTC |
| | | | AGCTCCCAGGTCACCCAGGAACTGAGGTGAGTG |
| | | | TCCCCATCCTGGCCCTTGACCCTCCTGGTGGGC |
| | | | GGCTATACCTCCCCAGGTCCAGGTTTCATTCTG |
| | | | CCCCTGTCGCTAAGTCTTGGGGGGCCTGGGTCT |
| | | | CTGCTGGTTCTAGCTTCCTCTTCCCATTTCTGA |
| | | | CTCCTGGCTTTAGCTCTCTGGAATTCTCTCTCT |
| | | | CAGCTTTGTCTCTCTCTCTTCCCTTCTGACTCA |
| | | | GTCTCTCACACTCGTCCTGGCTCTGTCTCTGTC |
| | | | CTTCCCTAGCTCTTTTATATAGAGACAGAGAGA |
| | | | TGGGGTCTCACTGTGTTGCCCAGGCTGGTCTTG |
| | | | AACTTCTGGGCTCAAGCGATCCTCCCGCCTCGG |
| | | | CCTCCCAAAGTGCTGGGATTAGAGGCATGAGCC |
| | | | ACCTTGCCCGGCCTCCTAGCTCCTTCTTCGTCT |
| | | | CTGCCTCTGCCCTCTGCATCTGCTCTCTGCATC |
| | | | TGTCTCTGTCTCCTTCTCTCGGCCTCTGCCCCG |
| | | | TTCCTTCTCTCCCTCTTGGGTCTCTCTGGCTCA |
| | | | TCCCCATCTCGCCCGCCCCATCCCAGCCCTTCT |
| | | | CCCCGCCTCCCACTGTGCGACACCCTCCCGCCC |
| | | | TCTCGGCCGCAGGGCGCTGATGGACGAGACCAT |
| | | | GAAGGAGTTGAAGGCCTACAAATCGGAACTGGA |
| | | | GGAACAACTGACCCCGGTGGCGGAGGAGACGCG |
| | | | GGCACGGCTGTCCAAGGAGCTGCAGGCGGCGCA |
| | | | GGCCCGGCTGGGCGCGGACATGGAGGACGTGTG |
| | | | CGGCCGCCTGGTGCAGTACCGCGGCGAGGTGCA |
| | | | GGCCATGCTCGGCCAGAGCACCGAGGAGCTGCG |
| | | | GGTGCGCCTCGCCTCCCACCTGCGCAAGCTGCG |
| | | | TAAGCGGCTCCTCCGCGATGCCGATGACCTGCA |
| | | | GAAGCGCCTGGCAGTGTACCAGGCCGGGGCCCG |
| | | | CGAGGGCGCCGAGCGCGGCCTCAGCGCCATCCG |
| | | | CGAGCGCCTGGGGCCCCTGGTGGAACAGGGCCG |
| | | | CGTGCGGGCCGCCACTGTGGGCTCCCTGGCCGG |
| | | | CCAGCCGCTACAGGAGCGGGCCCAGGCCTGGGG |
| | | | CGAGCGGCTGCGCGCGCGGATGGAGGAGATGGG |
| | | | CAGCCGGACCCGCGACCGCCTGGACGAGGTGAA |
| | | | GGAGCAGGTGGCGGAGGTGCGCGCCAAGCTGGA |
| | | | GGAGCAGGCCCAGCAGATACGCCTGCAGGCCGA |
| | | | GGCCTTCCAGGCCCGCCTCAAGAGCTGGTTCGA |
| | | | GCCCCTGGTGGAAGACATGCAGCGCCAGTGGGC |
| | | | CGGGCTGGTGGAGAAGGTGCAGGCTGCCGTGGG |
| | | | CACCAGCGCCGCCCCTGTGCCCAGCGACAATCA |
| | | | CTGAACGCCGAAGCCTGCAGCCATGCGACCCCA |
| | | | CGCCACCCCGTGCCTCCTGCCTCCGCGCAGCCT |
| | | | GCAGCGGGAGACCCTGTCCCCGCCCCAGCCGTC |
| | | | CTCCTGGGGTGGACCCTAGTTTAATAAAGATTC |
| | | | ACCAAGTTTCACGCATC |
| SEQ ID | DNA | pcDNA5/FRT | GACGGATCGGGAGATCTCCCGATCCCCTATGGT |
| NO. 158 | | | GCACTCTCAGTACAATCTGCTCTGATGCCGCAT |
| | | | AGTTAAGCCAGTATCTGCTCCCTGCTTGTGTGT |
| | | | TGGAGGTCGCTGAGTAGTGCGCGAGCAAAATTT |
| | | | AAGCTACAACAAGGCAAGGCTTGACCGACAATT |
| | | | GCATGAAGAATCTGCTTAGGGTTAGGCGTTTTG |
| | | | CGCTGCTTCGCGATGTACGGGCCAGATATACGC |
| | | | GTTGACATTGATTATTGACTAGTTATTAATAGT |
| | | | AATCAATTACGGGGTCATTAGTTCATAGCCCAT |
| | | | ATATGGAGTTCCGCGTTACATAACTTACGGTAA |
| | | | ATGGCCCGCCTGGCTGACCGCCCAACGACCCCC |
| | | | GCCCATTGACGTCAATAATGACGTATGTTCCCA |
| | | | TAGTAACGCCAATAGGGACTTTCCATTGACGTC |
| | | | AATGGGTGGAGTATTTACGGTAAACTGCCCACT |
| | | | TGGCAGTACATCAAGTGTATCATATGCCAAGTA |
| | | | CGCCCCCTATTGACGTCAATGACGGTAAATGGC |
| | | | CCGCCTGGCATTATGCCCAGTACATGACCTTAT |
| | | | GGGACTTTCCTACTTGGCAGTACATCTACGTAT |
| | | | TAGTCATCGCTATTACCATGGTGATGCGGTTTT |
| | | | GGCAGTACATCAATGGGCGTGGATAGCGGTTTG |
| | | | ACTCACGGGGATTTCCAAGTCTCCACCCCATTG |
| | | | ACGTCAATGGGAGTTTGTTTTGGCACCAAAATC |
| | | | AACGGGACTTTCCAAAATGTCGTAACAACTCCG |
| | | | CCCCATTGACGCAAATGGGCGGTAGGCGTGTAC |
| | | | GGTGGGAGGTCTATATAAGCAGAGCTCTCTGGC |
| | | | TAACTAGAGAACCCACTGCTTACTGGCTTATCG |
| | | | AAATTAATACGACTCACTATAGGGAGACCCAAG |
| | | | CTGGCTAGCGTTTAAACTTAAGCTTGGTACCGA |
| | | | GCTCGGATCCACTAGTCCAGTGTGGTGGAATTC |
| | | | TGCAGATATCCAGCACAGTGGCGGCCGCTCGAG |
| | | | TCTAGAGGGCCCGTTTAAACCCGCTGATCAGCC |
| | | | TCGACTGTGCCTTCTAGTTGCCAGCCATCTGTT |
| | | | GTTTGCCCCTCCCCCGTGCCTTCCTTGACCCTG |
| | | | GAAGGTGCCACTCCCACTGTCCTTTCCTAATAA |
| | | | AATGAGGAAATTGCATCGCATTGTCTGAGTAGG |
| | | | TGTCATTCTATTCTGGGGGGTGGGGTGGGGCAG |
| | | | GACAGCAAGGGGGAGGATTGGGAAGACAATAGC |
| | | | AGGCATGCTGGGGATGCGGTGGGCTCTATGGCT |
| | | | TCTGAGGCGGAAAGAACCAGCTGGGGCTCTAGG |
| | | | GGGTATCCCCACGCGCCCTGTAGCGGCGCATTA |
| | | | AGCGCGGCGGGTGTGGTGGTTACGCGCAGCGTG |
| | | | ACCGCTACACTTGCCAGCGCCCTAGCGCCCGCT |
| | | | CCTTTCGCTTTCTTCCCTTCCTTTCTCGCCACG |
| | | | TTCGCCGGCTTTCCCCGTCAAGCTCTAAATCGG |
| | | | GGGTCCCTTTAGGGTTCCGATTTAGTGCTTTAC |
| | | | GGCACCTCGACCCCAAAAAACTTGATTAGGGTG |
| | | | ATGGTTCACGTACCTAGAAGTTCCTATTCCGAA |
| | | | GTTCCTATTCTCTAGAAAGTATAGGAACTTCCT |
| | | | TGGCCAAAAAGCCTGAACTCACCGCGACGTCTG |
| | | | TCGAGAAGTTTCTGATCGAAAAGTTCGACAGCG |
| | | | TCTCCGACCTGATGCAGCTCTCGGAGGGCGAAG |
| | | | AATCTCGTGCTTTCAGCTTCGATGTAGGAGGGC |
| | | | GTGGATATGTCCTGCGGGTAAATAGCTGCGCCG |
| | | | ATGGTTTCTACAAAGATCGTTATGTTTATCGGC |
| | | | ACTTTGCATCGGCCGCGCTCCCGATTCCGGAAG |
| | | | TGCTTGACATTGGGGAATTCAGCGAGAGCCTGA |
| | | | CCTATTGCATCTCCCGCCGTGCACAGGGTGTCA |
| | | | CGTTGCAAGACCTGCCTGAAACCGAACTGCCCG |
| | | | CTGTTCTGCAGCCGGTCGCGGAGGCCATGGATG |
| | | | CGATCGCTGCGGCCGATCTTAGCCAGACGAGCG |
| | | | GGTTCGGCCCATTCGGACCGCAAGGAATCGGTC |
| | | | AATACACTACATGGCGTGATTTCATATGCGCGA |
| | | | TTGCTGATCCCCATGTGTATCACTGGCAAACTG |
| | | | TGATGGACGACACCGTCAGTGCGTCCGTCGCGC |
| | | | AGGCTCTCGATGAGCTGATGCTTTGGGCCGAGG |
| | | | ACTGCCCCGAAGTCCGGCACCTCGTGCACGCGG |
| | | | ATTTCGGCTCCAACAATGTCCTGACGGACAATG |
| | | | GCCGCATAACAGCGGTCATTGACTGGAGCGAGG |
| | | | CGATGTTCGGGGATTCCCAATACGAGGTCGCCA |
| | | | ACATCTTCTTCTGGAGGCCGTGGTTGGCTTGTA |
| | | | TGGAGCAGCAGACGCGCTACTTCGAGCGGAGGC |
| | | | ATCCGGAGCTTGCAGGATCGCCGCGGCTCCGGG |
| | | | CGTATATGCTCCGCATTGGTCTTGACCAACTCT |
| | | | ATCAGAGCTTGGTTGACGGCAATTTCGATGATG |
| | | | CAGCTTGGGCGCAGGGTCGATGCGACGCAATCG |
| | | | TCCGATCCGGAGCCGGGACTGTCGGGCGTACAC |
| | | | AAATCGCCCGCAGAAGCGCGGCCGTCTGGACCG |
| | | | ATGGCTGTGTAGAAGTACTCGCCGATAGTGGAA |
| | | | ACCGACGCCCCAGCACTCGTCCGAGGGCAAAGG |
| | | | AATAGCACGTACTACGAGATTTCGATTCCACCG |
| | | | CCGCCTTCTATGAAAGGTTGGGCTTCGGAATCG |
| | | | TTTTCCGGGACGCCGGCTGGATGATCCTCCAGC |
| | | | GCGGGGATCTCATGCTGGAGTTCTTCGCCCACC |
| | | | CCAACTTGTTTATTGCAGCTTATAATGGTTACA |
| | | | AATAAAGCAATAGCATCACAAATTTCACAAATA |
| | | | AAGCATTTTTTTCACTGCATTCTAGTTGTGGTT |
| | | | TGTCCAAACTCATCAATGTATCTTATCATGTCT |
| | | | GTATACCGTCGACCTCTAGCTAGAGCTTGGCGT |
| | | | AATCATGGTCATAGCTGTTTCCTGTGTGAAATT |
| | | | GTTATCCGCTCACAATTCCACACAACATACGAG |
| | | | CCGGAAGCATAAAGTGTAAAGCCTGGGGTGCCT |
| | | | AATGAGTGAGCTAACTCACATTAATTGCGTTGC |
| | | | GCTCACTGCCCGCTTTCCAGTCGGGAAACCTGT |
| | | | CGTGCCAGCTGCATTAATGAATCGGCCAACGCG |
| | | | CGGGGAGAGGCGGTTTGCGTATTGGGCGCTCTT |
| | | | CCGCTTCCTCGCTCACTGACTCGCTGCGCTCGG |
| | | | TCGTTCGGCTGCGGCGAGCGGTATCAGCTCACT |
| | | | CAAAGGCGGTAATACGGTTATCCACAGAATCAG |
| | | | GGGATAACGCAGGAAAGAACATGTGAGCAAAAG |
| | | | GCCAGCAAAAGGCCAGGAACCGTAAAAAGGCCG |
| | | | CGTTGCTGGCGTTTTTCCATAGGCTCCGCCCCC |
| | | | CTGACGAGCATCACAAAAATCGACGCTCAAGTC |
| | | | AGAGGTGGCGAAACCCGACAGGACTATAAAGAT |
| | | | ACCAGGCGTTTCCCCCTGGAAGCTCCCTCGTGC |
| | | | GCTCTCCTGTTCCGACCCTGCCGCTTACCGGAT |
| | | | ACCTGTCCGCCTTTCTCCCTTCGGGAAGCGTGG |
| | | | CGCTTTCTCATAGCTCACGCTGTAGGTATCTCA |
| | | | GTTCGGTGTAGGTCGTTCGCTCCAAGCTGGGCT |
| | | | GTGTGCACGAACCCCCCGTTCAGCCCGACCGCT |
| | | | GCGCCTTATCCGGTAACTATCGTCTTGAGTCCA |
| | | | ACCCGGTAAGACACGACTTATCGCCACTGGCAG |
| | | | CAGCCACTGGTAACAGGATTAGCAGAGCGAGGT |
| | | | ATGTAGGCGGTGCTACAGAGTTCTTGAAGTGGT |
| | | | GGCCTAACTACGGCTACACTAGAAGGACAGTAT |
| | | | TTGGTATCTGCGCTCTGCTGAAGCCAGTTACCT |
| | | | TCGGAAAAAGAGTTGGTAGCTCTTGATCCGGCA |
| | | | AACAAACCACCGCTGGTAGCGGTGGTTTTTTTG |
| | | | TTTGCAAGCAGCAGATTACGCGCAGAAAAAAAG |
| | | | GATCTCAAGAAGATCCTTTGATCTTTTCTACGG |
| | | | GGTCTGACGCTCAGTGGAACGAAAACTCACGTT |
| | | | AAGGGATTTTGGTCATGAGATTATCAAAAAGGA |
| | | | TCTTCACCTAGATCCTTTTAAATTAAAAATGAA |
| | | | GTTTTAAATCAATCTAAAGTATATATGAGTAAA |
| | | | CTTGGTCTGACAGTTACCAATGCTTAATCAGTG |
| | | | AGGCACCTATCTCAGCGATCTGTCTATTTCGTT |
| | | | CATCCATAGTTGCCTGACTCCCCGTCGTGTAGA |
| | | | TAACTACGATACGGGAGGGCTTACCATCTGGCC |
| | | | CCAGTGCTGCAATGATACCGCGAGACCCACGCT |
| | | | CACCGGCTCCAGATTTATCAGCAATAAACCAGC |
| | | | CAGCCGGAAGGGCCGAGCGCAGAAGTGGTCCTG |
| | | | CAACTTTATCCGCCTCCATCCAGTCTATTAATT |
| | | | GTTGCCGGGAAGCTAGAGTAAGTAGTTCGCCAG |
| | | | TTAATAGTTTGCGCAACGTTGTTGCCATTGCTA |
| | | | CAGGCATCGTGGTGTCACGCTCGTCGTTTGGTA |
| | | | TGGCTTCATTCAGCTCCGGTTCCCAACGATCAA |
| | | | GGCGAGTTACATGATCCCCCATGTTGTGCAAAA |
| | | | AAGCGGTTAGCTCCTTCGGTCCTCCGATCGTTG |
| | | | TCAGAAGTAAGTTGGCCGCAGTGTTATCACTCA |
| | | | TGGTTATGGCAGCACTGCATAATTCTCTTACTG |
| | | | TCATGCCATCCGTAAGATGCTTTTCTGTGACTG |
| | | | GTGAGTACTCAACCAAGTCATTCTGAGAATAGT |
| | | | GTATGCGGCGACCGAGTTGCTCTTGCCCGGCGT |
| | | | CAATACGGGATAATACCGCGCCACATAGCAGAA |
| | | | CTTTAAAAGTGCTCATCATTGGAAAACGTTCTT |
| | | | CGGGGCGAAAACTCTCAAGGATCTTACCGCTGT |
| | | | TGAGATCCAGTTCGATGTAACCCACTCGTGCAC |
| | | | CCAACTGATCTTCAGCATCTTTTACTTTCACCA |
| | | | GCGTTTCTGGGTGAGCAAAAACAGGAAGGCAAA |
| | | | ATGCCGCAAAAAAGGGAATAAGGGCGACACGGA |
| | | | AATGTTGAATACTCATACTCTTCCTTTTTCAAT |
| | | | ATTATTGAAGCATTTATCAGGGTTATTGTCTCA |
| | | | TGAGCGGATACATATTTGAATGTATTTAGAAAA |
| | | | ATAAACAAATAGGGGTTCCGCGCACATTTCCCC |
| | | | GAAAAGTGCCACCTGACGTC |
| SEQ ID | DNA | pb43 full length | GACTCTTCGCGATGTACGGGCCAGATATACGCG |
| NO. 159 | | | TTGACATTGATTATTGACTAGTTATTAATAGTA |
| | | | ATCAATTACGGGGTCATTAGTTCATAGCCCATA |
| | | | TATGGAGTTCCGCGTTACATAACTTACGGTAAA |
| | | | TGGCCCGCCTGGCTGACCGCCCAACGACCCCCG |
| | | | CCCATTGACGTCAATAATGACGTATGTTCCCAT |
| | | | AGTAACGCCAATAGGGACTTTCCATTGACGTCA |
| | | | ATGGGTGGACTATTTACGGTAAACTGCCCACTT |
| | | | GGCAGTACATCAAGTGTATCATATGCCAAGTAC |
| | | | GCCCCCTATTGACGTCAATGACGGTAAATGGCC |
| | | | CGCCTGGCATTATGCCCAGTACATGACCTTATG |
| | | | GGACTTTCCTACTTGGCAGTACATCTACGTATT |
| | | | AGTCATCGCTATTACCATGGTGATGCGGTTTTG |
| | | | GCAGTACATCAATGGGCGTGGATAGCGGTTTGA |
| | | | CTCACGGGGATTTCCAAGTCTCCACCCCATTGA |
| | | | CGTCAATGGGAGTTTGTTTTGGCACCAAAATCA |
| | | | ACGGGACTTTCCAAAATGTCGTAACAACTCCGC |
| | | | CCCATTGACGCAAATGGGCGGTAGGCGTGTACG |
| | | | GTGGGAGGTCTATATAAGCAGAGCTCTCTGGCT |
| | | | AACTAGAGAACCCACTGCTTACTGGCTTATCGA |
| | | | AATTAATACGACTCACTATAGGGAGACCCAAGC |
| | | | TGGCTAGCGTTTAAACTTAAGCTTGGGGTACCG |
| | | | GCGGCGATGGCGGCGATGGCCGAGCGGCCCTTC |
| | | | CAGTGCAGGATCTGTATGCGCAACTTTTCTCGG |
| | | | TCCTCCAACCTGACCCGGCACATCAGAACCCAT |
| | | | ACAGGCGAAAAGCCTTTCGCCTGCGACATTTGT |
| | | | GGGAGAAAATTTGCTCGGTCCGACGCCCTGTCC |
| | | | GAGCATACCAAGATCCACACCGGCTCTCAGAAA |
| | | | CCATTCCAGTGCCGCATTTGTATGCGGAATTTT |
| | | | TCCGACTCCTCCGCCCTGACCACCCATATCCGC |
| | | | ACTCACACCGGAGAGAAGCCCTTTGCTTGCGAC |
| | | | ATTTGTGGCAGGAAATTTGCTGACTCCTCCGAC |
| | | | CTGTCCGAGCACACTAAGATCCATACTGGGTCA |
| | | | CAGAAACCTTTCCAGTGCCGGATTTGTATGAGA |
| | | | AACTTTAGCCAGTCCGGCAACCTGTCCCAGCAC |
| | | | ATCAGAACCCATACAGGCGAAAAGCCTTTCGCC |
| | | | TGCGACATTTGTGGGAGAAAATTTGCTGACCGG |
| | | | TCCGACCTGACCCGGCATACCAAGATCCACACC |
| | | | GGCTCTCAGAAACCATTCCAGTGCCGCATTTGT |
| | | | ATGCGGAATTTTTCCCGGTCCGACAACCTGACC |
| | | | CGGCACATCAGAACACATACTGGGCTGAGAGGA |
| | | | TCCAATTCTGGTGATCCTCGGAGACACAGTCTG |
| | | | GGCGGTTCTCGTAAACCCGATCTGATTGCCTAT |
| | | | AAAAACTTTGATCTGCTGGTCATTGTTCTTAAG |
| | | | CCTTGAGCGGCCGCTCGAGTCTAGAGGGCCCGT |
| | | | TTAAACCCGCTGATCAGCCTCGACTGTGCCTTC |
| | | | TAGTTGCCAGCCATCTGTTGTTTGCCCCTCCCC |
| | | | CGTGCCTTCCTTGACCCTGGAAGGTGCCACTCC |
| | | | CACTGTCCTTTCCTAATAAAATGAGGAAATTGC |
| | | | ATCGCATTGTCTGAGTAGGTGTCATTCTATTCT |
| | | | GGGGGGTGGGGTGGGGCAGGACAGCAAGGGGGA |
| | | | GGATTGGGAAGACAATAGCAGGCATGCTGGGGA |
| | | | TGCGGTGGGCTCTATGGCTTCTACTGGGCGGTT |
| | | | TTATGGACAGCAAGCGAACCGGAATTGCCAGCT |
| | | | GGGGCGCCCTCTGGTAAGGTTGGGAAGCCCTGC |
| | | | AAAGTAAACTGGATGGCTTTCTCGCCGCCAAGG |
| | | | ATCTGATGGCGCAGGGGATCAAGCTCTGATCAA |
| | | | GAGACAGGATGAGGATCGTTTCGCATGATTGAA |
| | | | CAAGATGGATTGCACGCAGGTTCTCCGGCCGCT |
| | | | TGGGTGGAGAGGCTATTCGGCTATGACTGGGCA |
| | | | CAACAGACAATCGGCTGCTCTGATGCCGCCGTG |
| | | | TTCCGGCTGTCAGCGCAGGGGCGCCCGGTTCTT |
| | | | TTTGTCAAGACCGACCTGTCCGGTGCCCTGAAT |
| | | | GAACTGCAAGACGAGGCAGCGCGGCTATCGTGG |
| | | | CTGGCCACGACGGGCGTTCCTTGCGCAGCTGTG |
| | | | CTCGACGTTGTCACTGAAGCGGGAAGGGACTGG |
| | | | CTGCTATTGGGCGAAGTGCCGGGGCAGGATCTC |
| | | | CTGTCATCTCACCTTGCTCCTGCCGAGAAAGTA |
| | | | TCCATCATGGCTGATGCAATGCGGCGGCTGCAT |
| | | | ACGCTTGATCCGGCTACCTGCCCATTCGACCAC |
| | | | CAAGCGAAACATCGCATCGAGCGAGCACGTACT |
| | | | CGGATGGAAGCCGGTCTTGTCGATCAGGATGAT |
| | | | CTGGACGAAGAGCATCAGGGGCTCGCGCCAGCC |
| | | | GAACTGTTCGCCAGGCTCAAGGCGAGCATGCCC |
| | | | GACGGCGAGGATCTCGTCGTGACCCATGGCGAT |
| | | | GCCTGCTTGCCGAATATCATGGTGGAAAATGGC |
| | | | CGCTTTTCTGGATTCATCGACTGTGGCCGGCTG |
| | | | GGTGTGGCGGACCGCTATCAGGACATAGCGTTG |
| | | | GCTACCCGTGATATTGCTGAAGAGCTTGGCGGC |
| | | | GAATGGGCTGACCGCTTCCTCGTGCTTTACGGT |
| | | | ATCGCCGCTCCCGATTCGCAGCGCATCGCCTTC |
| | | | TATCGCCTTCTTGACGAGTTCTTCTGAATTATT |
| | | | AACGCTTACAATTTCCTGATGCGGTATTTTCTC |
| | | | CTTACGCATCTGTGCGGTATTTCACACCGCATA |
| | | | CAGGTGGCACTTTTCGGGGAAATGTGCGCGGAA |
| | | | CCCCTATTTGTTTATTTTTCTAAATACATTCAA |
| | | | ATATGTATCCGCTCATGAGACAATAACCCTGAT |
| | | | AAATGCTTCAATAATAGCACGTGCTAAAACTTC |
| | | | ATTTTTAATTTAAAAGGATCTAGGTGAAGATCC |
| | | | TTTTTGATAATCTCATGACCAAAATCCCTTAAC |
| | | | GTGAGTTTTCGTTCCACTGAGCGTCAGACCCCG |
| | | | TAGAAAAGATCAAAGGATCTTCTTGAGATCCTT |
| | | | TTTTTCTGCGCGTAATCTGCTGCTTGCAAACAA |
| | | | AAAAACCACCGCTACCAGCGGTGGTTTGTTTGC |
| | | | CGGATCAAGAGCTACCAACTCTTTTTCCGAAGG |
| | | | TAACTGGCTTCAGCAGAGCGCAGATACCAAATA |
| | | | CTGTCCTTCTAGTGTAGCCGTAGTTAGGCCACC |
| | | | ACTTCAAGAACTCTGTAGCACCGCCTACATACC |
| | | | TCGCTCTGCTAATCCTGTTACCAGTGGCTGCTG |
| | | | CCAGTGGCGATAAGTCGTGTCTTACCGGGTTGG |
| | | | ACTCAAGACGATAGTTACCGGATAAGGCGCAGC |
| | | | GGTCGGGCTGAACGGGGGGTTCGTGCACACAGC |
| | | | CCAGCTTGGAGCGAACGACCTACACCGAACTGA |
| | | | GATACCTACAGCGTGAGCTATGAGAAAGCGCCA |
| | | | CGCTTCCCGAAGGGAGAAAGGCGGACAGGTATC |
| | | | CGGTAAGCGGCAGGGTCGGAACAGGAGAGCGCA |
| | | | CGAGGGAGCTTCCAGGGGGAAACGCCTGGTATC |
| | | | TTTATAGTCCTGTCGGGTTTCGCCACCTCTGAC |
| | | | TTGAGCGTCGATTTTTGTGATGCTCGTCAGGGG |
| | | | GGCGGAGCCTATGGAAAAACGCCAGCAACGCGG |
| | | | CCTTTTTACGGTTCCTGGGCTTTTGCTGGCCTT |
| | | | TTGCTCACATGTTCTT |
| SEQ ID | DNA | pb43 cDNA | ATGGCGGCGATGGCCGAGCGGCCCTTCCAGTGC |
| NO. 160 | | | AGGATCTGTATGCGCAACTTTTCTCGGTCCTCC |
| | | | AACCTGACCCGGCACATCAGAACCCATACAGGC |
| | | | GAAAAGCCTTTCGCCTGCGACATTTGTGGGAGA |
| | | | AAATTTGCTCGGTCCGACGCCCTGTCCGAGCAT |
| | | | ACCAAGATCCACACCGGCTCTCAGAAACCATTC |
| | | | CAGTGCCGCATTTGTATGCGGAATTTTTCCGAC |
| | | | TCCTCCGCCCTGACCACCCATATCCGCACTCAC |
| | | | ACCGGAGAGAAGCCCTTTGCTTGCGACATTTGT |
| | | | GGCAGGAAATTTGCTGACTCCTCCGACCTGTCC |
| | | | GAGCACACTAAGATCCATACTGGGTCACAGAAA |
| | | | CCTTTCCAGTGCCGGATTTGTATGAGAAACTTT |
| | | | AGCCAGTCCGGCAACCTGTCCCAGCACATCAGA |
| | | | ACCCATACAGGCGAAAAGCCTTTCGCCTGCGAC |
| | | | ATTTGTGGGAGAAAATTTGCTGACCGGTCCGAC |
| | | | CTGACCCGGCATACCAAGATCCACACCGGCTCT |
| | | | CAGAAACCATTCCAGTGCCGCATTTGTATGCGG |
| | | | AATTTTTCCCGGTCCGACAACCTGACCCGGCAC |
| | | | ATCAGAACACATACTGGGCTGAGAGGATCCAAT |
| | | | TCTGGTGATCCTCGGAGACACAGTCTGGGCGGT |
| | | | TCTCGTAAACCCGATCTGATTGCCTATAAAAAC |
| | | | TTTGATCTGCTGGTCATTGTTCTTAAGCCTTGA |
| SEQ ID | Amino | pb43 DLR Amino | MAAMAERPFQCRICMRNESRSSNLTRHIRTHTG |
| NO. 161 | acids | acids | EKPFACDICGRKFARSDALSEHTKIHTGSQKPF |
| | | | QCRICMRNFSDSSALTTHIRTHTGEKPFACDIC |
| | | | GRKFADSSDLSEHTKIHTGSQKPFQCRICMRNF |
| | | | SQSGNLSQHIRTHTGEKPFACDICGRKFADRSD |
| | | | LTRHTKIHTGSQKPFQCRICMRNFSRSDNLTRH |
| | | | IRTHTGLRGSNSGDPRRHSLGGSRKPDLIAYKN |
| | | | FDLLVIVLKP* |
| SEQ ID | DNA | pb43 DLR | 5′-GAG-GCC-AAA-CCC-TTC-CTG-GAG-3′ |
| NO. 162 | | recognition | |
| | | sequence | |
| SEQ ID | DNA | Pop79 BCL11A | CTCTTAGACATAACACACCAGGGTCAATACAAC |
| NO. 163 | | ODN donor | TTTGAAGCTAGTCTAGTGCAAGCTAACAGTTGC |
| | | | TTGAATTCACAGGCTCCAGGAAGGGTTTGGCCT |
| | | | CTGATTAGGGTGGGGGCGTGGGTGGGGTAGAAG |
| | | | AGGACTGGC |
| SEQ ID | DNA | Pop75 BCL11A F | ACTCTTAGACATAACACACC |
| NO. 164 | | | |
| SEQ ID | DNA | Pop76 BCL11A R | AAGAGAGCCTTCCGAAAGA |
| NO. 165 | | | |
| SEQ ID | DNA | pb46 full length | GACTCTTCGCGATGTACGGGCCAGATATACGCG |
| NO. 166 | | | TTGACATTGATTATTGACTAGTTATTAATAGTA |
| | | | ATCAATTACGGGGTCATTAGTTCATAGCCCATA |
| | | | TATGGAGTTCCGCGTTACATAACTTACGGTAAA |
| | | | TGGCCCGCCTGGCTGACCGCCCAACGACCCCCG |
| | | | CCCATTGACGTCAATAATGACGTATGTTCCCAT |
| | | | AGTAACGCCAATAGGGACTTTCCATTGACGTCA |
| | | | ATGGGTGGACTATTTACGGTAAACTGCCCACTT |
| | | | GGCAGTACATCAAGTGTATCATATGCCAAGTAC |
| | | | GCCCCCTATTGACGTCAATGACGGTAAATGGCC |
| | | | CGCCTGGCATTATGCCCAGTACATGACCTTATG |
| | | | GGACTTTCCTACTTGGCAGTACATCTACGTATT |
| | | | AGTCATCGCTATTACCATGGTGATGCGGTTTTG |
| | | | GCAGTACATCAATGGGCGTGGATAGCGGTTTGA |
| | | | CTCACGGGGATTTCCAAGTCTCCACCCCATTGA |
| | | | CGTCAATGGGAGTTTGTTTTGGCACCAAAATCA |
| | | | ACGGGACTTTCCAAAATGTCGTAACAACTCCGC |
| | | | CCCATTGACGCAAATGGGCGGTAGGCGTGTACG |
| | | | GTGGGAGGTCTATATAAGCAGAGCTCTCTGGCT |
| | | | AACTAGAGAACCCACTGCTTACTGGCTTATCGA |
| | | | AATTAATACGACTCACTATAGGGAGACCCAAGC |
| | | | TGGCTAGCGTTTAAACTTAAGCTTATGGACTAC |
| | | | AAAGACCATGACGGTGATTATAAAGATCATGAC |
| | | | ATCGATTACAAGGATGACGATGACAAGATGGCC |
| | | | CCCAAGAAGAAGAGGAAGGTGGGCATTCACGGG |
| | | | GTACCGATGGCGGCGATGGCCGAGCGGCCCTTC |
| | | | CAGTGCAGGATCTGTATGCGCAACTTTTCTCGG |
| | | | TCCTCCAACCTGACCCGGCACATCAGAACCCAT |
| | | | ACAGGCGAAAAGCCTTTCGCCTGCGACATTTGT |
| | | | GGGAGAAAATTTGCTCGGTCCGACGCCCTGTCC |
| | | | GAGCATACCAAGATCCACACCGGCTCTCAGAAA |
| | | | CCATTCCAGTGCCGCATTTGTATGCGGAATTTT |
| | | | TCCGACTCCTCCGCCCTGACCACCCATATCCGC |
| | | | ACTCACACCGGAGAGAAGCCCTTTGCTTGCGAC |
| | | | ATTTGTGGCAGGAAATTTGCTGACTCCTCCGAC |
| | | | CTGTCCGAGCACACTAAGATCCATACTGGGTCA |
| | | | CAGAAACCTTTCCAGTGCCGGATTTGTATGAGA |
| | | | AACTTTAGCCAGTCCGGCAACCTGTCCCAGCAC |
| | | | ATCAGAACCCATACAGGCGAAAAGCCTTTCGCC |
| | | | TGCGACATTTGTGGGAGAAAATTTGCTGACCGG |
| | | | TCCGACCTGACCCGGCATACCAAGATCCACACC |
| | | | GGCTCTCAGAAACCATTCCAGTGCCGCATTTGT |
| | | | ATGCGGAATTTTTCCCGGTCCGACAACCTGACC |
| | | | CGGCACATCAGAACACATACTGGGCTGAGAGGA |
| | | | TCCGGCGGCGGCGGCGGCTCCGGCGGCGGCGGC |
| | | | GGCTCCGGCGGCGGCGGCGGCTCCGGCGGCGGC |
| | | | GGCGGCTCCGGCGGCGGCGGCGGCTCCGGCGGC |
| | | | GGCGGCGGCTCCATGGCGGCGATGGCCGAGCGG |
| | | | CCCTTCCAGTGCAGGATCTGTATGCGCAACTTT |
| | | | TCTCGGTCCGACCACCTGACCCGGCACATCAGA |
| | | | ACCCATACAGGCGAAAAGCCTTTCGCCTGCGAC |
| | | | ATTTGTGGGAGAAAATTTGCTACCTCCGGCCAC |
| | | | CTGACCCGGCATACCAAGATCCACACCGGCTCT |
| | | | CAGAAACCATTCCAGTGCCGCATTTGTATGCGG |
| | | | AATTTTTCCCGGTCCGACGCCCTGACCCGGCAT |
| | | | ATCCGCACTCACACCGGAGAGAAGCCCTTTGCT |
| | | | TGCGACATTTGTGGCAGGAAATTTGCTGACCGG |
| | | | TCCCACCTGACCCGGCACACTAAGATCCATACT |
| | | | GGGTCACAGAAACCTTTCCAGTGCCGGATTTGT |
| | | | ATGAGAAACTTTAGCCGGTCCGACCACCTGACC |
| | | | CGGCACATCAGAACCCATACAGGCGAAAAGCCT |
| | | | TTCGCCTGCGACATTTGTGGGAGAAAATTTGCT |
| | | | ACCTCCGGCCACCTGACCCGGCATACCAAGATC |
| | | | CACACCGGCTCTCAGAAACCATTCCAGTGCCGC |
| | | | ATTTGTATGCGGAATTTTTCCCGGTCCGACAAC |
| | | | CTGACCACCCACATCAGAACACATACTGGGCTG |
| | | | AGATGAGCGGCCGCTCGAGTCTAGAGGGCCCGT |
| | | | TTAAACCCGCTGATCAGCCTCGACTGTGCCTTC |
| | | | TAGTTGCCAGCCATCTGTTGTTTGCCCCTCCCC |
| | | | CGTGCCTTCCTTGACCCTGGAAGGTGCCACTCC |
| | | | CACTGTCCTTTCCTAATAAAATGAGGAAATTGC |
| | | | ATCGCATTGTCTGAGTAGGTGTCATTCTATTCT |
| | | | GGGGGGTGGGGTGGGGCAGGACAGCAAGGGGGA |
| | | | GGATTGGGAAGACAATAGCAGGCATGCTGGGGA |
| | | | TGCGGTGGGCTCTATGGCTTCTACTGGGCGGTT |
| | | | TTATGGACAGCAAGCGAACCGGAATTGCCAGCT |
| | | | GGGGCGCCCTCTGGTAAGGTTGGGAAGCCCTGC |
| | | | AAAGTAAACTGGATGGCTTTCTCGCCGCCAAGG |
| | | | ATCTGATGGCGCAGGGGATCAAGCTCTGATCAA |
| | | | GAGACAGGATGAGGATCGTTTCGCATGATTGAA |
| | | | CAAGATGGATTGCACGCAGGTTCTCCGGCCGCT |
| | | | TGGGTGGAGAGGCTATTCGGCTATGACTGGGCA |
| | | | CAACAGACAATCGGCTGCTCTGATGCCGCCGTG |
| | | | TTCCGGCTGTCAGCGCAGGGGCGCCCGGTTCTT |
| | | | TTTGTCAAGACCGACCTGTCCGGTGCCCTGAAT |
| | | | GAACTGCAAGACGAGGCAGCGCGGCTATCGTGG |
| | | | CTGGCCACGACGGGCGTTCCTTGCGCAGCTGTG |
| | | | CTCGACGTTGTCACTGAAGCGGGAAGGGACTGG |
| | | | CTGCTATTGGGCGAAGTGCCGGGGCAGGATCTC |
| | | | CTGTCATCTCACCTTGCTCCTGCCGAGAAAGTA |
| | | | TCCATCATGGCTGATGCAATGCGGCGGCTGCAT |
| | | | ACGCTTGATCCGGCTACCTGCCCATTCGACCAC |
| | | | CAAGCGAAACATCGCATCGAGCGAGCACGTACT |
| | | | CGGATGGAAGCCGGTCTTGTCGATCAGGATGAT |
| | | | CTGGACGAAGAGCATCAGGGGCTCGCGCCAGCC |
| | | | GAACTGTTCGCCAGGCTCAAGGCGAGCATGCCC |
| | | | GACGGCGAGGATCTCGTCGTGACCCATGGCGAT |
| | | | GCCTGCTTGCCGAATATCATGGTGGAAAATGGC |
| | | | CGCTTTTCTGGATTCATCGACTGTGGCCGGCTG |
| | | | GGTGTGGCGGACCGCTATCAGGACATAGCGTTG |
| | | | GCTACCCGTGATATTGCTGAAGAGCTTGGCGGC |
| | | | GAATGGGCTGACCGCTTCCTCGTGCTTTACGGT |
| | | | ATCGCCGCTCCCGATTCGCAGCGCATCGCCTTC |
| | | | TATCGCCTTCTTGACGAGTTCTTCTGAATTATT |
| | | | AACGCTTACAATTTCCTGATGCGGTATTTTCTC |
| | | | CTTACGCATCTGTGCGGTATTTCACACCGCATA |
| | | | CAGGTGGCACTTTTCGGGGAAATGTGCGCGGAA |
| | | | CCCCTATTTGTTTATTTTTCTAAATACATTCAA |
| | | | ATATGTATCCGCTCATGAGACAATAACCCTGAT |
| | | | AAATGCTTCAATAATAGCACGTGCTAAAACTTC |
| | | | ATTTTTAATTTAAAAGGATCTAGGTGAAGATCC |
| | | | TTTTTGATAATCTCATGACCAAAATCCCTTAAC |
| | | | GTGAGTTTTCGTTCCACTGAGCGTCAGACCCCG |
| | | | TAGAAAAGATCAAAGGATCTTCTTGAGATCCTT |
| | | | TTTTTCTGCGCGTAATCTGCTGCTTGCAAACAA |
| | | | AAAAACCACCGCTACCAGCGGTGGTTTGTTTGC |
| | | | CGGATCAAGAGCTACCAACTCTTTTTCCGAAGG |
| | | | TAACTGGCTTCAGCAGAGCGCAGATACCAAATA |
| | | | CTGTCCTTCTAGTGTAGCCGTAGTTAGGCCACC |
| | | | ACTTCAAGAACTCTGTAGCACCGCCTACATACC |
| | | | TCGCTCTGCTAATCCTGTTACCAGTGGCTGCTG |
| | | | CCAGTGGCGATAAGTCGTGTCTTACCGGGTTGG |
| | | | ACTCAAGACGATAGTTACCGGATAAGGCGCAGC |
| | | | GGTCGGGCTGAACGGGGGGTTCGTGCACACAGC |
| | | | CCAGCTTGGAGCGAACGACCTACACCGAACTGA |
| | | | GATACCTACAGCGTGAGCTATGAGAAAGCGCCA |
| | | | CGCTTCCCGAAGGGAGAAAGGCGGACAGGTATC |
| | | | CGGTAAGCGGCAGGGTCGGAACAGGAGAGCGCA |
| | | | CGAGGGAGCTTCCAGGGGGAAACGCCTGGTATC |
| | | | TTTATAGTCCTGTCGGGTTTCGCCACCTCTGAC |
| | | | TTGAGCGTCGATTTTTGTGATGCTCGTCAGGGG |
| | | | GGCGGAGCCTATGGAAAAACGCCAGCAACGCGG |
| | | | CCTTTTTACGGTTCCTGGGCTTTTGCTGGCCTT |
| | | | TTGCTCACATGTTCTT |
| SEQ ID | DNA | pb46 cDNA | ATGGACTACAAAGACCATGACGGTGATTATAAA |
| NO. 167 | | | GATCATGACATCGATTACAAGGATGACGATGAC |
| | | | AAGATGGCCCCCAAGAAGAAGAGGAAGGTGGGC |
| | | | ATTCACGGGGTACCGATGGCGGCGATGGCCGAG |
| | | | CGGCCCTTCCAGTGCAGGATCTGTATGCGCAAC |
| | | | TTTTCTCGGTCCTCCAACCTGACCCGGCACATC |
| | | | AGAACCCATACAGGCGAAAAGCCTTTCGCCTGC |
| | | | GACATTTGTGGGAGAAAATTTGCTCGGTCCGAC |
| | | | GCCCTGTCCGAGCATACCAAGATCCACACCGGC |
| | | | TCTCAGAAACCATTCCAGTGCCGCATTTGTATG |
| | | | CGGAATTTTTCCGACTCCTCCGCCCTGACCACC |
| | | | CATATCCGCACTCACACCGGAGAGAAGCCCTTT |
| | | | GCTTGCGACATTTGTGGCAGGAAATTTGCTGAC |
| | | | TCCTCCGACCTGTCCGAGCACACTAAGATCCAT |
| | | | ACTGGGTCACAGAAACCTTTCCAGTGCCGGATT |
| | | | TGTATGAGAAACTTTAGCCAGTCCGGCAACCTG |
| | | | TCCCAGCACATCAGAACCCATACAGGCGAAAAG |
| | | | CCTTTCGCCTGCGACATTTGTGGGAGAAAATTT |
| | | | GCTGACCGGTCCGACCTGACCCGGCATACCAAG |
| | | | ATCCACACCGGCTCTCAGAAACCATTCCAGTGC |
| | | | CGCATTTGTATGCGGAATTTTTCCCGGTCCGAC |
| | | | AACCTGACCCGGCACATCAGAACACATACTGGG |
| | | | CTGAGAGGATCCGGCGGCGGCGGCGGCTCCGGC |
| | | | GGCGGCGGCGGCTCCGGCGGCGGCGGCGGCTCC |
| | | | GGCGGCGGCGGCGGCTCCGGCGGCGGCGGCGGC |
| | | | TCCGGCGGCGGCGGCGGCTCCATGGCGGCGATG |
| | | | GCCGAGCGGCCCTTCCAGTGCAGGATCTGTATG |
| | | | CGCAACTTTTCTCGGTCCGACCACCTGACCCGG |
| | | | CACATCAGAACCCATACAGGCGAAAAGCCTTTC |
| | | | GCCTGCGACATTTGTGGGAGAAAATTTGCTACC |
| | | | TCCGGCCACCTGACCCGGCATACCAAGATCCAC |
| | | | ACCGGCTCTCAGAAACCATTCCAGTGCCGCATT |
| | | | TGTATGCGGAATTTTTCCCGGTCCGACGCCCTG |
| | | | ACCCGGCATATCCGCACTCACACCGGAGAGAAG |
| | | | CCCTTTGCTTGCGACATTTGTGGCAGGAAATTT |
| | | | GCTGACCGGTCCCACCTGACCCGGCACACTAAG |
| | | | ATCCATACTGGGTCACAGAAACCTTTCCAGTGC |
| | | | CGGATTTGTATGAGAAACTTTAGCCGGTCCGAC |
| | | | CACCTGACCCGGCACATCAGAACCCATACAGGC |
| | | | GAAAAGCCTTTCGCCTGCGACATTTGTGGGAGA |
| | | | AAATTTGCTACCTCCGGCCACCTGACCCGGCAT |
| | | | ACCAAGATCCACACCGGCTCTCAGAAACCATTC |
| | | | CAGTGCCGCATTTGTATGCGGAATTTTTCCCGG |
| | | | TCCGACAACCTGACCACCCACATCAGAACACAT |
| | | | ACTGGGCTGAGATGA |
| SEQ ID | Amino | pb46 DLR Amino | MDYKDHDGDYKDHDIDYKDDDDKMAPKKKRKVG |
| NO. 168 | acids | acids | IHGVPMAAMAERPFQCRICMRNFSRSSNLTRHI |
| | | | RTHTGEKPFACDICGRKFARSDALSEHTKIHTG |
| | | | SQKPFQCRICMRNESDSSALTTHIRTHTGEKPF |
| | | | ACDICGRKFADSSDLSEHTKIHTGSQKPFQCRI |
| | | | CMRNFSQSGNLSQHIRTHTGEKPFACDICGRKF |
| | | | ADRSDLTRHTKIHTGSQKPFQCRICMRNFSRSD |
| | | | NLTRHIRTHTGLRGSGGGGGSGGGGGSGGGGGS |
| | | | GGGGGSGGGGGSGGGGGSMAAMAERPFQCRICM |
| | | | RNFSRSDHLTRHIRTHTGEKPFACDICGRKFAT |
| | | | SGHLTRHTKIHTGSQKPFQCRICMRNFSRSDAL |
| | | | TRHIRTHTGEKPFACDICGRKFADRSHLTRHTK |
| | | | IHTGSQKPFQCRICMRNFSRSDHLTRHIRTHTG |
| | | | EKPFACDICGRKFATSGHLTRHTKIHTGSQKPF |
| | | | QCRICMRNFSRSDNLTTHIRTHTGLR* |
| SEQ ID | DNA | pb46 R element | 5′-TAG-GGT-GGG-GGC-GTG-GGT-GGG |
| NO. 169 | | recognition | |
| | | sequence | |
| SEQ ID | DNA | Pop113 BCL11A | TGATTCCAGTGCAAAGTCCA |
| NO. 170 | | Far F | |
| SEQ ID | DNA | Pop114 BCL11A | AGAGAGCCTTCCGAAAGAGG |
| NO. 171 | | Far R | |
| SEQ ID | DNA | pb49 full length | GACTCTTCGCGATGTACGGGCCAGATATACGCG |
| NO. 172 | | | TTGACATTGATTATTGACTAGTTATTAATAGTA |
| | | | ATCAATTACGGGGTCATTAGTTCATAGCCCATA |
| | | | TATGGAGTTCCGCGTTACATAACTTACGGTAAA |
| | | | TGGCCCGCCTGGCTGACCGCCCAACGACCCCCG |
| | | | CCCATTGACGTCAATAATGACGTATGTTCCCAT |
| | | | AGTAACGCCAATAGGGACTTTCCATTGACGTCA |
| | | | ATGGGTGGACTATTTACGGTAAACTGCCCACTT |
| | | | GGCAGTACATCAAGTGTATCATATGCCAAGTAC |
| | | | GCCCCCTATTGACGTCAATGACGGTAAATGGCC |
| | | | CGCCTGGCATTATGCCCAGTACATGACCTTATG |
| | | | GGACTTTCCTACTTGGCAGTACATCTACGTATT |
| | | | AGTCATCGCTATTACCATGGTGATGCGGTTTTG |
| | | | GCAGTACATCAATGGGCGTGGATAGCGGTTTGA |
| | | | CTCACGGGGATTTCCAAGTCTCCACCCCATTGA |
| | | | CGTCAATGGGAGTTTGTTTTGGCACCAAAATCA |
| | | | ACGGGACTTTCCAAAATGTCGTAACAACTCCGC |
| | | | CCCATTGACGCAAATGGGCGGTAGGCGTGTACG |
| | | | GTGGGAGGTCTATATAAGCAGAGCTCTCTGGCT |
| | | | AACTAGAGAACCCACTGCTTACTGGCTTATCGA |
| | | | AATTAATACGACTCACTATAGGGAGACCCAAGC |
| | | | TGGCTAGCGTTTAAACTTAAGCTTGGGGTACCG |
| | | | GCGGCGATGGCGGCGATGGCCGAGCGGCCCTTC |
| | | | GCCTGCGACATTTGTGGGAGAAAATTTGCTGAT |
| | | | CAGTCCGGCAACCTGACCCGGCATACCAAGATC |
| | | | CACACCGGCTCTCAGAAACCATTCCAGTGCAGG |
| | | | ATCTGTATGCGCAACTTTTCTCGGTCCGACAAC |
| | | | CTGTCCCAGCACATCAGAACCCATACAGGCGAA |
| | | | AAGCCTTTTGCTTGCGACATTTGTGGCAGGAAA |
| | | | TTTGCTACCTCCGGCGACCTGTCCCAGCACACT |
| | | | AAGATCCATACTGGGTCACAGAAACCTTTCCAG |
| | | | TGCCGCATTTGTATGCGGAATTTTTCCACCTCC |
| | | | GGCTCCCTGACCCGGCATATCCGCACTCACACC |
| | | | GGAGAGAAGCCCTTTGCATGCGACATTTGTGGA |
| | | | CGGAAATTTGCTCGGTCCGACGCCCTGACCCGG |
| | | | CATACCAAGATTCACACTGGGTCTCAGAAACCT |
| | | | TTCCAGTGCAGGATTTGTATGAGAAATTTTTCC |
| | | | ACCTCCGGCGACCTGTCCGAGCACATCAGAACC |
| | | | CATACAGGCGAAAAGCCTTTTGCTTGCGACATT |
| | | | TGTGGCAGGAAATTTGCTCAGTCCGGCAACCTG |
| | | | TCCGAGCACACTAAGATCCATACTGGGTCACAG |
| | | | AAACCTTTCCAGTGCCGCATTTGTATGCGGAAT |
| | | | TTTTCCCAGTCCGGCGACCTGTCCCAGCACATC |
| | | | AGAACCCATACAGGCGAAAAGCCTTTTGCTTGC |
| | | | GACATTTGTGGCAGGAAATTTGCTCGGTCCTCC |
| | | | GCCCTGACCCGGCACACTAAGATCCATACTGGG |
| | | | TCACAGAAACCTTTCCAGTGCCGCATTTGTATG |
| | | | CGGAATTTTTCCCGGTCCGACGCCCTGTCCGAG |
| | | | CACATCAGAACACATACTGGGCTGAGAGGATCC |
| | | | AATTCTGGTGATCCTCGGAGACACAGTCTGGGC |
| | | | GGTTCTCGTAAACCCGATCTGATTGCCTATAAA |
| | | | AACTTTGATCTGCTGGTCATTGTTCTTAAGCCT |
| | | | TGAGCGGCCGCTCGAGTCTAGAGGGCCCGTTTA |
| | | | AACCCGCTGATCAGCCTCGACTGTGCCTTCTAG |
| | | | TTGCCAGCCATCTGTTGTTTGCCCCTCCCCCGT |
| | | | GCCTTCCTTGACCCTGGAAGGTGCCACTCCCAC |
| | | | TGTCCTTTCCTAATAAAATGAGGAAATTGCATC |
| | | | GCATTGTCTGAGTAGGTGTCATTCTATTCTGGG |
| | | | GGGTGGGGTGGGGCAGGACAGCAAGGGGGAGGA |
| | | | TTGGGAAGACAATAGCAGGCATGCTGGGGATGC |
| | | | GGTGGGCTCTATGGCTTCTACTGGGCGGTTTTA |
| | | | TGGACAGCAAGCGAACCGGAATTGCCAGCTGGG |
| | | | GCGCCCTCTGGTAAGGTTGGGAAGCCCTGCAAA |
| | | | GTAAACTGGATGGCTTTCTCGCCGCCAAGGATC |
| | | | TGATGGCGCAGGGGATCAAGCTCTGATCAAGAG |
| | | | ACAGGATGAGGATCGTTTCGCATGATTGAACAA |
| | | | GATGGATTGCACGCAGGTTCTCCGGCCGCTTGG |
| | | | GTGGAGAGGCTATTCGGCTATGACTGGGCACAA |
| | | | CAGACAATCGGCTGCTCTGATGCCGCCGTGTTC |
| | | | CGGCTGTCAGCGCAGGGGCGCCCGGTTCTTTTT |
| | | | GTCAAGACCGACCTGTCCGGTGCCCTGAATGAA |
| | | | CTGCAAGACGAGGCAGCGCGGCTATCGTGGCTG |
| | | | GCCACGACGGGCGTTCCTTGCGCAGCTGTGCTC |
| | | | GACGTTGTCACTGAAGCGGGAAGGGACTGGCTG |
| | | | CTATTGGGCGAAGTGCCGGGGCAGGATCTCCTG |
| | | | TCATCTCACCTTGCTCCTGCCGAGAAAGTATCC |
| | | | ATCATGGCTGATGCAATGCGGCGGCTGCATACG |
| | | | CTTGATCCGGCTACCTGCCCATTCGACCACCAA |
| | | | GCGAAACATCGCATCGAGCGAGCACGTACTCGG |
| | | | ATGGAAGCCGGTCTTGTCGATCAGGATGATCTG |
| | | | GACGAAGAGCATCAGGGGCTCGCGCCAGCCGAA |
| | | | CTGTTCGCCAGGCTCAAGGCGAGCATGCCCGAC |
| | | | GGCGAGGATCTCGTCGTGACCCATGGCGATGCC |
| | | | TGCTTGCCGAATATCATGGTGGAAAATGGCCGC |
| | | | TTTTCTGGATTCATCGACTGTGGCCGGCTGGGT |
| | | | GTGGCGGACCGCTATCAGGACATAGCGTTGGCT |
| | | | ACCCGTGATATTGCTGAAGAGCTTGGCGGCGAA |
| | | | TGGGCTGACCGCTTCCTCGTGCTTTACGGTATC |
| | | | GCCGCTCCCGATTCGCAGCGCATCGCCTTCTAT |
| | | | CGCCTTCTTGACGAGTTCTTCTGAATTATTAAC |
| | | | GCTTACAATTTCCTGATGCGGTATTTTCTCCTT |
| | | | ACGCATCTGTGCGGTATTTCACACCGCATACAG |
| | | | GTGGCACTTTTCGGGGAAATGTGCGCGGAACCC |
| | | | CTATTTGTTTATTTTTCTAAATACATTCAAATA |
| | | | TGTATCCGCTCATGAGACAATAACCCTGATAAA |
| | | | TGCTTCAATAATAGCACGTGCTAAAACTTCATT |
| | | | TTTAATTTAAAAGGATCTAGGTGAAGATCCTTT |
| | | | TTGATAATCTCATGACCAAAATCCCTTAACGTG |
| | | | AGTTTTCGTTCCACTGAGCGTCAGACCCCGTAG |
| | | | AAAAGATCAAAGGATCTTCTTGAGATCCTTTTT |
| | | | TTCTGCGCGTAATCTGCTGCTTGCAAACAAAAA |
| | | | AACCACCGCTACCAGCGGTGGTTTGTTTGCCGG |
| | | | ATCAAGAGCTACCAACTCTTTTTCCGAAGGTAA |
| | | | CTGGCTTCAGCAGAGCGCAGATACCAAATACTG |
| | | | TCCTTCTAGTGTAGCCGTAGTTAGGCCACCACT |
| | | | TCAAGAACTCTGTAGCACCGCCTACATACCTCG |
| | | | CTCTGCTAATCCTGTTACCAGTGGCTGCTGCCA |
| | | | GTGGCGATAAGTCGTGTCTTACCGGGTTGGACT |
| | | | CAAGACGATAGTTACCGGATAAGGCGCAGCGGT |
| | | | CGGGCTGAACGGGGGGTTCGTGCACACAGCCCA |
| | | | GCTTGGAGCGAACGACCTACACCGAACTGAGAT |
| | | | ACCTACAGCGTGAGCTATGAGAAAGCGCCACGC |
| | | | TTCCCGAAGGGAGAAAGGCGGACAGGTATCCGG |
| | | | TAAGCGGCAGGGTCGGAACAGGAGAGCGCACGA |
| | | | GGGAGCTTCCAGGGGGAAACGCCTGGTATCTTT |
| | | | ATAGTCCTGTCGGGTTTCGCCACCTCTGACTTG |
| | | | AGCGTCGATTTTTGTGATGCTCGTCAGGGGGGC |
| | | | GGAGCCTATGGAAAAACGCCAGCAACGCGGCCT |
| | | | TTTTACGGTTCCTGGGCTTTTGCTGGCCTTTTG |
| | | | CTCACATGTTCTT |
| SEQ ID | DNA | pb49 cDNA | ATGGCGGCGATGGCCGAGCGGCCCTTCGCCTGC |
| NO. 173 | | | GACATTTGTGGGAGAAAATTTGCTGATCAGTCC |
| | | | GGCAACCTGACCCGGCATACCAAGATCCACACC |
| | | | GGCTCTCAGAAACCATTCCAGTGCAGGATCTGT |
| | | | ATGCGCAACTTTTCTCGGTCCGACAACCTGTCC |
| | | | CAGCACATCAGAACCCATACAGGCGAAAAGCCT |
| | | | TTTGCTTGCGACATTTGTGGCAGGAAATTTGCT |
| | | | ACCTCCGGCGACCTGTCCCAGCACACTAAGATC |
| | | | CATACTGGGTCACAGAAACCTTTCCAGTGCCGC |
| | | | ATTTGTATGCGGAATTTTTCCACCTCCGGCTCC |
| | | | CTGACCCGGCATATCCGCACTCACACCGGAGAG |
| | | | AAGCCCTTTGCATGCGACATTTGTGGACGGAAA |
| | | | TTTGCTCGGTCCGACGCCCTGACCCGGCATACC |
| | | | AAGATTCACACTGGGTCTCAGAAACCTTTCCAG |
| | | | TGCAGGATTTGTATGAGAAATTTTTCCACCTCC |
| | | | GGCGACCTGTCCGAGCACATCAGAACCCATACA |
| | | | GGCGAAAAGCCTTTTGCTTGCGACATTTGTGGC |
| | | | AGGAAATTTGCTCAGTCCGGCAACCTGTCCGAG |
| | | | CACACTAAGATCCATACTGGGTCACAGAAACCT |
| | | | TTCCAGTGCCGCATTTGTATGCGGAATTTTTCC |
| | | | CAGTCCGGCGACCTGTCCCAGCACATCAGAACC |
| | | | CATACAGGCGAAAAGCCTTTTGCTTGCGACATT |
| | | | TGTGGCAGGAAATTTGCTCGGTCCTCCGCCCTG |
| | | | ACCCGGCACACTAAGATCCATACTGGGTCACAG |
| | | | AAACCTTTCCAGTGCCGCATTTGTATGCGGAAT |
| | | | TTTTCCCGGTCCGACGCCCTGTCCGAGCACATC |
| | | | AGAACACATACTGGGCTGAGAGGATCCAATTCT |
| | | | GGTGATCCTCGGAGACACAGTCTGGGCGGTTCT |
| | | | CGTAAACCCGATCTGATTGCCTATAAAAACTTT |
| | | | GATCTGCTGGTCATTGTTCTTAAGCCTTGA |
| SEQ ID | Amino | pb49 DLR Amino | MAAMAERPFACDICGRKFADQSGNLTRHTKIHT |
| NO. 174 | acids | acids | GSQKPFQCRICMRNFSRSDNLSQHIRTHTGEKP |
| | | | FACDICGRKFATSGDLSQHTKIHTGSQKPFQCR |
| | | | ICMRNFSTSGSLTRHIRTHTGEKPFACDICGRK |
| | | | FARSDALTRHTKIHTGSQKPFQCRICMRNESTS |
| | | | GDLSEHIRTHTGEKPFACDICGRKFAQSGNLSE |
| | | | HTKIHTGSQKPFQCRICMRNFSQSGDLSQHIRT |
| | | | HTGEKPFACDICGRKFARSSALTRHTKIHTGSQ |
| | | | KPFQCRICMRNFSRSDALSEHIRTHTGLRGSNS |
| | | | GDPRRHSLGGSRKPDLIAYKNFDLLVIVLKP* |
| SEQ ID | DNA | pb49 DLR | 5′-CTG-GTG-ACA-CAA-CCT-GTG-GTT- |
| NO. 175 | | recognition | ACT-AAG-GAA |
| | | sequence | |
| SEQ ID | DNA | Pop88 DMD Odn F | TAATTTTTCTTTTTCTTCTTTTTTCCTTTTTGC |
| NO. 176 | | | AAAAACCCAAAATATTTTAGCTCCTACTCAGAC |
| | | | TGTTAGACTCTGGTGACACAACCTGTGGTTACT |
| | | | AAGGAAACTGCCATCTCCAAACTAGAAATGCCA |
| | | | TCTTCC |
| SEQ ID | DNA | Pop83 DMD F (out) | TTGGCTCTTTAGCTTGTGTTTC |
| NO. 177 | | | |
| SEQ ID | DNA | Pop84 DMD R (in) | GGCATTTCTAGTTTGGAGATGG |
| NO. 178 | | | |
| SEQ ID | DNA | pb52 full length | GACTCTTCGCGATGTACGGGCCAGATATACGCG |
| NO. 179 | | | TTGACATTGATTATTGACTAGTTATTAATAGTA |
| | | | ATCAATTACGGGGTCATTAGTTCATAGCCCATA |
| | | | TATGGAGTTCCGCGTTACATAACTTACGGTAAA |
| | | | TGGCCCGCCTGGCTGACCGCCCAACGACCCCCG |
| | | | CCCATTGACGTCAATAATGACGTATGTTCCCAT |
| | | | AGTAACGCCAATAGGGACTTTCCATTGACGTCA |
| | | | ATGGGTGGACTATTTACGGTAAACTGCCCACTT |
| | | | GGCAGTACATCAAGTGTATCATATGCCAAGTAC |
| | | | GCCCCCTATTGACGTCAATGACGGTAAATGGCC |
| | | | CGCCTGGCATTATGCCCAGTACATGACCTTATG |
| | | | GGACTTTCCTACTTGGCAGTACATCTACGTATT |
| | | | AGTCATCGCTATTACCATGGTGATGCGGTTTTG |
| | | | GCAGTACATCAATGGGCGTGGATAGCGGTTTGA |
| | | | CTCACGGGGATTTCCAAGTCTCCACCCCATTGA |
| | | | CGTCAATGGGAGTTTGTTTTGGCACCAAAATCA |
| | | | ACGGGACTTTCCAAAATGTCGTAACAACTCCGC |
| | | | CCCATTGACGCAAATGGGCGGTAGGCGTGTACG |
| | | | GTGGGAGGTCTATATAAGCAGAGCTCTCTGGCT |
| | | | AACTAGAGAACCCACTGCTTACTGGCTTATCGA |
| | | | AATTAATACGACTCACTATAGGGAGACCCAAGC |
| | | | TGGCTAGCGTTTAAACTTAAGCTTATGGACTAC |
| | | | AAAGACCATGACGGTGATTATAAAGATCATGAC |
| | | | ATCGATTACAAGGATGACGATGACAAGATGGCC |
| | | | CCCAAGAAGAAGAGGAAGGTGGGCATTCACGGG |
| | | | GTACCGGCGGCGATGGCGGCGATGGCCGAGCGG |
| | | | CCCTTCCAGTGCAGGATCTGTATGCGCAACTTT |
| | | | TCTCAGTCCGGCGACCTGACCCGGCACATCAGA |
| | | | ACCCATACAGGCGAAAAGCCTTTCGCCTGCGAC |
| | | | ATTTGTGGGAGAAAATTTGCTCGGTCCGACAAC |
| | | | CTGTCCGAGCATACCAAGATCCACACCGGCTCT |
| | | | CAGAAACCATTCCAGTGCCGCATTTGTATGCGG |
| | | | AATTTTTCCGACCGGTCCGCCCTGTCCGAGCAT |
| | | | ATCCGCACTCACACCGGAGAGAAGCCCTTTGCT |
| | | | TGCGACATTTGTGGCAGGAAATTTGCTCGGTCC |
| | | | TCCGCCCTGTCCGAGCACACTAAGATCCATACT |
| | | | GGGTCACAGAAACCTTTCCAGTGCCGGATTTGT |
| | | | ATGAGAAACTTTAGCCGGTCCTCCCACCTGACC |
| | | | CGGCACATCAGAACCCATACAGGCGAAAAGCCT |
| | | | TTCGCCTGCGACATTTGTGGGAGAAAATTTGCT |
| | | | CGGTCCGACGCCCTGACCCGGCATACCAAGATC |
| | | | CACACCGGCTCTCAGAAACCATTCCAGTGCCGC |
| | | | ATTTGTATGCGGAATTTTTCCCGGTCCGACGCC |
| | | | CTGTCCGAGCACATCAGAACACATACTGGGCTG |
| | | | AGAGGATCCGGCGGCGGCGGCGGCTCCGGCGGC |
| | | | GGCGGCGGCTCCGGCGGCGGCGGCGGCTCCGGC |
| | | | GGCGGCGGCGGCTCCGGCGGCGGCGGCGGCTCC |
| | | | GGCGGCGGCGGCGGCTCCATGGCGGCGATGGCC |
| | | | GAGCGGCCCTTCCAGTGCAGGATCTGTATGCGC |
| | | | AACTTTTCTCAGTCCGGCCACCTGACCCGGCAC |
| | | | ATCAGAACCCATACAGGCGAAAAGCCTTTCGCC |
| | | | TGCGACATTTGTGGGAGAAAATTTGCTCGGTCC |
| | | | GACGCCCTGACCCGGCATACCAAGATCCACACC |
| | | | GGCTCTCAGAAACCATTCCAGTGCCGCATTTGT |
| | | | ATGCGGAATTTTTCCACCTCCGGCGACCTGTCC |
| | | | GAGCATATCCGCACTCACACCGGAGAGAAGCCC |
| | | | TTTGCTTGCGACATTTGTGGCAGGAAATTTGCT |
| | | | CGGTCCTCCGACCTGACCCGGCACACTAAGATC |
| | | | CATACTGGGTCACAGAAACCTTTCCAGTGCCGG |
| | | | ATTTGTATGAGAAACTTTAGCCGGTCCGACCAC |
| | | | CTGTCCCAGCACATCAGAACCCATACAGGCGAA |
| | | | AAGCCTTTCGCCTGCGACATTTGTGGGAGAAAA |
| | | | TTTGCTGACCGGTCCGACCTGACCCGGCATACC |
| | | | AAGATCCACACCGGCTCTCAGAAACCATTCCAG |
| | | | TGCCGCATTTGTATGCGGAATTTTTCCCGGTCC |
| | | | GACGCCCTGTCCGAGCACATCAGAACACATACT |
| | | | GGGCTGAGATGAGCGGCCGCTCGAGTCTAGAGG |
| | | | GCCCGTTTAAACCCGCTGATCAGCCTCGACTGT |
| | | | GCCTTCTAGTTGCCAGCCATCTGTTGTTTGCCC |
| | | | CTCCCCCGTGCCTTCCTTGACCCTGGAAGGTGC |
| | | | CACTCCCACTGTCCTTTCCTAATAAAATGAGGA |
| | | | AATTGCATCGCATTGTCTGAGTAGGTGTCATTC |
| | | | TATTCTGGGGGGTGGGGTGGGGCAGGACAGCAA |
| | | | GGGGGAGGATTGGGAAGACAATAGCAGGCATGC |
| | | | TGGGGATGCGGTGGGCTCTATGGCTTCTACTGG |
| | | | GCGGTTTTATGGACAGCAAGCGAACCGGAATTG |
| | | | CCAGCTGGGGCGCCCTCTGGTAAGGTTGGGAAG |
| | | | CCCTGCAAAGTAAACTGGATGGCTTTCTCGCCG |
| | | | CCAAGGATCTGATGGCGCAGGGGATCAAGCTCT |
| | | | GATCAAGAGACAGGATGAGGATCGTTTCGCATG |
| | | | ATTGAACAAGATGGATTGCACGCAGGTTCTCCG |
| | | | GCCGCTTGGGTGGAGAGGCTATTCGGCTATGAC |
| | | | TGGGCACAACAGACAATCGGCTGCTCTGATGCC |
| | | | GCCGTGTTCCGGCTGTCAGCGCAGGGGCGCCCG |
| | | | GTTCTTTTTGTCAAGACCGACCTGTCCGGTGCC |
| | | | CTGAATGAACTGCAAGACGAGGCAGCGCGGCTA |
| | | | TCGTGGCTGGCCACGACGGGCGTTCCTTGCGCA |
| | | | GCTGTGCTCGACGTTGTCACTGAAGCGGGAAGG |
| | | | GACTGGCTGCTATTGGGCGAAGTGCCGGGGCAG |
| | | | GATCTCCTGTCATCTCACCTTGCTCCTGCCGAG |
| | | | AAAGTATCCATCATGGCTGATGCAATGCGGCGG |
| | | | CTGCATACGCTTGATCCGGCTACCTGCCCATTC |
| | | | GACCACCAAGCGAAACATCGCATCGAGCGAGCA |
| | | | CGTACTCGGATGGAAGCCGGTCTTGTCGATCAG |
| | | | GATGATCTGGACGAAGAGCATCAGGGGCTCGCG |
| | | | CCAGCCGAACTGTTCGCCAGGCTCAAGGCGAGC |
| | | | ATGCCCGACGGCGAGGATCTCGTCGTGACCCAT |
| | | | GGCGATGCCTGCTTGCCGAATATCATGGTGGAA |
| | | | AATGGCCGCTTTTCTGGATTCATCGACTGTGGC |
| | | | CGGCTGGGTGTGGCGGACCGCTATCAGGACATA |
| | | | GCGTTGGCTACCCGTGATATTGCTGAAGAGCTT |
| | | | GGCGGCGAATGGGCTGACCGCTTCCTCGTGCTT |
| | | | TACGGTATCGCCGCTCCCGATTCGCAGCGCATC |
| | | | GCCTTCTATCGCCTTCTTGACGAGTTCTTCTGA |
| | | | ATTATTAACGCTTACAATTTCCTGATGCGGTAT |
| | | | TTTCTCCTTACGCATCTGTGCGGTATTTCACAC |
| | | | CGCATACAGGTGGCACTTTTCGGGGAAATGTGC |
| | | | GCGGAACCCCTATTTGTTTATTTTTCTAAATAC |
| | | | ATTCAAATATGTATCCGCTCATGAGACAATAAC |
| | | | CCTGATAAATGCTTCAATAATAGCACGTGCTAA |
| | | | AACTTCATTTTTAATTTAAAAGGATCTAGGTGA |
| | | | AGATCCTTTTTGATAATCTCATGACCAAAATCC |
| | | | CTTAACGTGAGTTTTCGTTCCACTGAGCGTCAG |
| | | | ACCCCGTAGAAAAGATCAAAGGATCTTCTTGAG |
| | | | ATCCTTTTTTTCTGCGCGTAATCTGCTGCTTGC |
| | | | AAACAAAAAAACCACCGCTACCAGCGGTGGTTT |
| | | | GTTTGCCGGATCAAGAGCTACCAACTCTTTTTC |
| | | | CGAAGGTAACTGGCTTCAGCAGAGCGCAGATAC |
| | | | CAAATACTGTCCTTCTAGTGTAGCCGTAGTTAG |
| | | | GCCACCACTTCAAGAACTCTGTAGCACCGCCTA |
| | | | CATACCTCGCTCTGCTAATCCTGTTACCAGTGG |
| | | | CTGCTGCCAGTGGCGATAAGTCGTGTCTTACCG |
| | | | GGTTGGACTCAAGACGATAGTTACCGGATAAGG |
| | | | CGCAGCGGTCGGGCTGAACGGGGGGTTCGTGCA |
| | | | CACAGCCCAGCTTGGAGCGAACGACCTACACCG |
| | | | AACTGAGATACCTACAGCGTGAGCTATGAGAAA |
| | | | GCGCCACGCTTCCCGAAGGGAGAAAGGCGGACA |
| | | | GGTATCCGGTAAGCGGCAGGGTCGGAACAGGAG |
| | | | AGCGCACGAGGGAGCTTCCAGGGGGAAACGCCT |
| | | | GGTATCTTTATAGTCCTGTCGGGTTTCGCCACC |
| | | | TCTGACTTGAGCGTCGATTTTTGTGATGCTCGT |
| | | | CAGGGGGGCGGAGCCTATGGAAAAACGCCAGCA |
| | | | ACGCGGCCTTTTTACGGTTCCTGGGCTTTTGCT |
| | | | GGCCTTTTGCTCACATGTTCTT |
| SEQ ID | DNA | pb52 cDNA | ATGGACTACAAAGACCATGACGGTGATTATAAA |
| NO. 180 | | | GATCATGACATCGATTACAAGGATGACGATGAC |
| | | | AAGATGGCCCCCAAGAAGAAGAGGAAGGTGGGC |
| | | | ATTCACGGGGTACCGGCGGCGATGGCGGCGATG |
| | | | GCCGAGCGGCCCTTCCAGTGCAGGATCTGTATG |
| | | | CGCAACTTTTCTCAGTCCGGCGACCTGACCCGG |
| | | | CACATCAGAACCCATACAGGCGAAAAGCCTTTC |
| | | | GCCTGCGACATTTGTGGGAGAAAATTTGCTCGG |
| | | | TCCGACAACCTGTCCGAGCATACCAAGATCCAC |
| | | | ACCGGCTCTCAGAAACCATTCCAGTGCCGCATT |
| | | | TGTATGCGGAATTTTTCCGACCGGTCCGCCCTG |
| | | | TCCGAGCATATCCGCACTCACACCGGAGAGAAG |
| | | | CCCTTTGCTTGCGACATTTGTGGCAGGAAATTT |
| | | | GCTCGGTCCTCCGCCCTGTCCGAGCACACTAAG |
| | | | ATCCATACTGGGTCACAGAAACCTTTCCAGTGC |
| | | | CGGATTTGTATGAGAAACTTTAGCCGGTCCTCC |
| | | | CACCTGACCCGGCACATCAGAACCCATACAGGC |
| | | | GAAAAGCCTTTCGCCTGCGACATTTGTGGGAGA |
| | | | AAATTTGCTCGGTCCGACGCCCTGACCCGGCAT |
| | | | ACCAAGATCCACACCGGCTCTCAGAAACCATTC |
| | | | CAGTGCCGCATTTGTATGCGGAATTTTTCCCGG |
| | | | TCCGACGCCCTGTCCGAGCACATCAGAACACAT |
| | | | ACTGGGCTGAGAGGATCCGGCGGCGGCGGCGGC |
| | | | TCCGGCGGCGGCGGCGGCTCCGGCGGCGGCGGC |
| | | | GGCTCCGGCGGCGGCGGCGGCTCCGGCGGCGGC |
| | | | GGCGGCTCCGGCGGCGGCGGCGGCTCCATGGCG |
| | | | GCGATGGCCGAGCGGCCCTTCCAGTGCAGGATC |
| | | | TGTATGCGCAACTTTTCTCAGTCCGGCCACCTG |
| | | | ACCCGGCACATCAGAACCCATACAGGCGAAAAG |
| | | | CCTTTCGCCTGCGACATTTGTGGGAGAAAATTT |
| | | | GCTCGGTCCGACGCCCTGACCCGGCATACCAAG |
| | | | ATCCACACCGGCTCTCAGAAACCATTCCAGTGC |
| | | | CGCATTTGTATGCGGAATTTTTCCACCTCCGGC |
| | | | GACCTGTCCGAGCATATCCGCACTCACACCGGA |
| | | | GAGAAGCCCTTTGCTTGCGACATTTGTGGCAGG |
| | | | AAATTTGCTCGGTCCTCCGACCTGACCCGGCAC |
| | | | ACTAAGATCCATACTGGGTCACAGAAACCTTTC |
| | | | CAGTGCCGGATTTGTATGAGAAACTTTAGCCGG |
| | | | TCCGACCACCTGTCCCAGCACATCAGAACCCAT |
| | | | ACAGGCGAAAAGCCTTTCGCCTGCGACATTTGT |
| | | | GGGAGAAAATTTGCTGACCGGTCCGACCTGACC |
| | | | CGGCATACCAAGATCCACACCGGCTCTCAGAAA |
| | | | CCATTCCAGTGCCGCATTTGTATGCGGAATTTT |
| | | | TCCCGGTCCGACGCCCTGTCCGAGCACATCAGA |
| | | | ACACATACTGGGCTGAGATGA |
| SEQ ID | Amino | pb52 DLR Amino | MDYKDHDGDYKDHDIDYKDDDDKMAPKKKRKVG |
| NO. 181 | acids | acids | IHGVPAAMAAMAERPFQCRICMRNFSQSGDLTR |
| | | | HIRTHTGEKPFACDICGRKFARSDNLSEHTKIH |
| | | | TGSQKPFQCRICMRNFSDRSALSEHIRTHTGEK |
| | | | PFACDICGRKFARSSALSEHTKIHTGSQKPFQC |
| | | | RICMRNFSRSSHLTRHIRTHTGEKPFACDICGR |
| | | | KFARSDALTRHTKIHTGSQKPFQCRICMRNFSR |
| | | | SDALSEHIRTHTGLRGSGGGGGSGGGGGSGGGG |
| | | | GSGGGGGSGGGGGSGGGGGSMAAMAERPFQCRI |
| | | | CMRNFSQSGHLTRHIRTHTGEKPFACDICGRKF |
| | | | ARSDALTRHTKIHTGSQKPFQCRICMRNFSTSG |
| | | | DLSEHIRTHTGEKPFACDICGRKFARSSDLTRH |
| | | | TKIHTGSQKPFQCRICMRNFSRSDHLSQHIRTH |
| | | | TGEKPFACDICGRKFADRSDLTRHTKIHTGSQK |
| | | | PFQCRICMRNFSRSDALSEHIRTHTGLR* |
| SEQ ID | DNA | pb53 full length | GACTCTTCGCGATGTACGGGCCAGATATACGCG |
| NO. 182 | | | TTGACATTGATTATTGACTAGTTATTAATAGTA |
| | | | ATCAATTACGGGGTCATTAGTTCATAGCCCATA |
| | | | TATGGAGTTCCGCGTTACATAACTTACGGTAAA |
| | | | TGGCCCGCCTGGCTGACCGCCCAACGACCCCCG |
| | | | CCCATTGACGTCAATAATGACGTATGTTCCCAT |
| | | | AGTAACGCCAATAGGGACTTTCCATTGACGTCA |
| | | | ATGGGTGGACTATTTACGGTAAACTGCCCACTT |
| | | | GGCAGTACATCAAGTGTATCATATGCCAAGTAC |
| | | | GCCCCCTATTGACGTCAATGACGGTAAATGGCC |
| | | | CGCCTGGCATTATGCCCAGTACATGACCTTATG |
| | | | GGACTTTCCTACTTGGCAGTACATCTACGTATT |
| | | | AGTCATCGCTATTACCATGGTGATGCGGTTTTG |
| | | | GCAGTACATCAATGGGCGTGGATAGCGGTTTGA |
| | | | CTCACGGGGATTTCCAAGTCTCCACCCCATTGA |
| | | | CGTCAATGGGAGTTTGTTTTGGCACCAAAATCA |
| | | | ACGGGACTTTCCAAAATGTCGTAACAACTCCGC |
| | | | CCCATTGACGCAAATGGGCGGTAGGCGTGTACG |
| | | | GTGGGAGGTCTATATAAGCAGAGCTCTCTGGCT |
| | | | AACTAGAGAACCCACTGCTTACTGGCTTATCGA |
| | | | AATTAATACGACTCACTATAGGGAGACCCAAGC |
| | | | TGGCTAGCGTTTAAACTTAAGCTTGGGGTACCG |
| | | | GCGGCGATGGCGGCGATGGCCGAGCGGCCCTTC |
| | | | CAGTGCAGGATCTGTATGCGCAACTTTTCTCAG |
| | | | TCCGGCGACCTGACCCGGCACATCAGAACCCAT |
| | | | ACAGGCGAAAAGCCTTTCGCCTGCGACATTTGT |
| | | | GGGAGAAAATTTGCTCGGTCCGACAACCTGTCC |
| | | | GAGCATACCAAGATCCACACCGGCTCTCAGAAA |
| | | | CCATTCCAGTGCCGCATTTGTATGCGGAATTTT |
| | | | TCCGACCGGTCCGCCCTGTCCGAGCATATCCGC |
| | | | ACTCACACCGGAGAGAAGCCCTTTGCTTGCGAC |
| | | | ATTTGTGGCAGGAAATTTGCTCGGTCCTCCGCC |
| | | | CTGTCCGAGCACACTAAGATCCATACTGGGTCA |
| | | | CAGAAACCTTTCCAGTGCCGGATTTGTATGAGA |
| | | | AACTTTAGCCGGTCCTCCCACCTGACCCGGCAC |
| | | | ATCAGAACCCATACAGGCGAAAAGCCTTTCGCC |
| | | | TGCGACATTTGTGGGAGAAAATTTGCTCGGTCC |
| | | | GACGCCCTGACCCGGCATACCAAGATCCACACC |
| | | | GGCTCTCAGAAACCATTCCAGTGCCGCATTTGT |
| | | | ATGCGGAATTTTTCCCGGTCCGACGCCCTGTCC |
| | | | GAGCACATCAGAACACATACTGGGCTGAGAGGA |
| | | | TCCAATTCTGGTGATCCTCGGAGACACAGTCTG |
| | | | GGCGGTTCTCGTAAACCCGATCTGATTGCCTAT |
| | | | AAAAACTTTGATCTGCTGGTCATTGTTCTTAAG |
| | | | CCTTGAGCGGCCGCTCGAGTCTAGAGGGCCCGT |
| | | | TTAAACCCGCTGATCAGCCTCGACTGTGCCTTC |
| | | | TAGTTGCCAGCCATCTGTTGTTTGCCCCTCCCC |
| | | | CGTGCCTTCCTTGACCCTGGAAGGTGCCACTCC |
| | | | CACTGTCCTTTCCTAATAAAATGAGGAAATTGC |
| | | | ATCGCATTGTCTGAGTAGGTGTCATTCTATTCT |
| | | | GGGGGGTGGGGTGGGGCAGGACAGCAAGGGGGA |
| | | | GGATTGGGAAGACAATAGCAGGCATGCTGGGGA |
| | | | TGCGGTGGGCTCTATGGCTTCTACTGGGCGGTT |
| | | | TTATGGACAGCAAGCGAACCGGAATTGCCAGCT |
| | | | GGGGCGCCCTCTGGTAAGGTTGGGAAGCCCTGC |
| | | | AAAGTAAACTGGATGGCTTTCTCGCCGCCAAGG |
| | | | ATCTGATGGCGCAGGGGATCAAGCTCTGATCAA |
| | | | GAGACAGGATGAGGATCGTTTCGCATGATTGAA |
| | | | CAAGATGGATTGCACGCAGGTTCTCCGGCCGCT |
| | | | TGGGTGGAGAGGCTATTCGGCTATGACTGGGCA |
| | | | CAACAGACAATCGGCTGCTCTGATGCCGCCGTG |
| | | | TTCCGGCTGTCAGCGCAGGGGCGCCCGGTTCTT |
| | | | TTTGTCAAGACCGACCTGTCCGGTGCCCTGAAT |
| | | | GAACTGCAAGACGAGGCAGCGCGGCTATCGTGG |
| | | | CTGGCCACGACGGGCGTTCCTTGCGCAGCTGTG |
| | | | CTCGACGTTGTCACTGAAGCGGGAAGGGACTGG |
| | | | CTGCTATTGGGCGAAGTGCCGGGGCAGGATCTC |
| | | | CTGTCATCTCACCTTGCTCCTGCCGAGAAAGTA |
| | | | TCCATCATGGCTGATGCAATGCGGCGGCTGCAT |
| | | | ACGCTTGATCCGGCTACCTGCCCATTCGACCAC |
| | | | CAAGCGAAACATCGCATCGAGCGAGCACGTACT |
| | | | CGGATGGAAGCCGGTCTTGTCGATCAGGATGAT |
| | | | CTGGACGAAGAGCATCAGGGGCTCGCGCCAGCC |
| | | | GAACTGTTCGCCAGGCTCAAGGCGAGCATGCCC |
| | | | GACGGCGAGGATCTCGTCGTGACCCATGGCGAT |
| | | | GCCTGCTTGCCGAATATCATGGTGGAAAATGGC |
| | | | CGCTTTTCTGGATTCATCGACTGTGGCCGGCTG |
| | | | GGTGTGGCGGACCGCTATCAGGACATAGCGTTG |
| | | | GCTACCCGTGATATTGCTGAAGAGCTTGGCGGC |
| | | | GAATGGGCTGACCGCTTCCTCGTGCTTTACGGT |
| | | | ATCGCCGCTCCCGATTCGCAGCGCATCGCCTTC |
| | | | TATCGCCTTCTTGACGAGTTCTTCTGAATTATT |
| | | | AACGCTTACAATTTCCTGATGCGGTATTTTCTC |
| | | | CTTACGCATCTGTGCGGTATTTCACACCGCATA |
| | | | CAGGTGGCACTTTTCGGGGAAATGTGCGCGGAA |
| | | | CCCCTATTTGTTTATTTTTCTAAATACATTCAA |
| | | | ATATGTATCCGCTCATGAGACAATAACCCTGAT |
| | | | AAATGCTTCAATAATAGCACGTGCTAAAACTTC |
| | | | ATTTTTAATTTAAAAGGATCTAGGTGAAGATCC |
| | | | TTTTTGATAATCTCATGACCAAAATCCCTTAAC |
| | | | GTGAGTTTTCGTTCCACTGAGCGTCAGACCCCG |
| | | | TAGAAAAGATCAAAGGATCTTCTTGAGATCCTT |
| | | | TTTTTCTGCGCGTAATCTGCTGCTTGCAAACAA |
| | | | AAAAACCACCGCTACCAGCGGTGGTTTGTTTGC |
| | | | CGGATCAAGAGCTACCAACTCTTTTTCCGAAGG |
| | | | TAACTGGCTTCAGCAGAGCGCAGATACCAAATA |
| | | | CTGTCCTTCTAGTGTAGCCGTAGTTAGGCCACC |
| | | | ACTTCAAGAACTCTGTAGCACCGCCTACATACC |
| | | | TCGCTCTGCTAATCCTGTTACCAGTGGCTGCTG |
| | | | CCAGTGGCGATAAGTCGTGTCTTACCGGGTTGG |
| | | | ACTCAAGACGATAGTTACCGGATAAGGCGCAGC |
| | | | GGTCGGGCTGAACGGGGGGTTCGTGCACACAGC |
| | | | CCAGCTTGGAGCGAACGACCTACACCGAACTGA |
| | | | GATACCTACAGCGTGAGCTATGAGAAAGCGCCA |
| | | | CGCTTCCCGAAGGGAGAAAGGCGGACAGGTATC |
| | | | CGGTAAGCGGCAGGGTCGGAACAGGAGAGCGCA |
| | | | CGAGGGAGCTTCCAGGGGGAAACGCCTGGTATC |
| | | | TTTATAGTCCTGTCGGGTTTCGCCACCTCTGAC |
| | | | TTGAGCGTCGATTTTTGTGATGCTCGTCAGGGG |
| | | | GGCGGAGCCTATGGAAAAACGCCAGCAACGCGG |
| | | | CCTTTTTACGGTTCCTGGGCTTTTGCTGGCCTT |
| | | | TTGCTCACATGTTCTT |
| SEQ ID | DNA | pb53 cDNA | ATGGCGGCGATGGCCGAGCGGCCCTTCCAGTGC |
| NO. 183 | | | AGGATCTGTATGCGCAACTTTTCTCAGTCCGGC |
| | | | GACCTGACCCGGCACATCAGAACCCATACAGGC |
| | | | GAAAAGCCTTTCGCCTGCGACATTTGTGGGAGA |
| | | | AAATTTGCTCGGTCCGACAACCTGTCCGAGCAT |
| | | | ACCAAGATCCACACCGGCTCTCAGAAACCATTC |
| | | | CAGTGCCGCATTTGTATGCGGAATTTTTCCGAC |
| | | | CGGTCCGCCCTGTCCGAGCATATCCGCACTCAC |
| | | | ACCGGAGAGAAGCCCTTTGCTTGCGACATTTGT |
| | | | GGCAGGAAATTTGCTCGGTCCTCCGCCCTGTCC |
| | | | GAGCACACTAAGATCCATACTGGGTCACAGAAA |
| | | | CCTTTCCAGTGCCGGATTTGTATGAGAAACTTT |
| | | | AGCCGGTCCTCCCACCTGACCCGGCACATCAGA |
| | | | ACCCATACAGGCGAAAAGCCTTTCGCCTGCGAC |
| | | | ATTTGTGGGAGAAAATTTGCTCGGTCCGACGCC |
| | | | CTGACCCGGCATACCAAGATCCACACCGGCTCT |
| | | | CAGAAACCATTCCAGTGCCGCATTTGTATGCGG |
| | | | AATTTTTCCCGGTCCGACGCCCTGTCCGAGCAC |
| | | | ATCAGAACACATACTGGGCTGAGAGGATCCAAT |
| | | | TCTGGTGATCCTCGGAGACACAGTCTGGGCGGT |
| | | | TCTCGTAAACCCGATCTGATTGCCTATAAAAAC |
| | | | TTTGATCTGCTGGTCATTGTTCTTAAGCCTTGA |
| SEQ ID | Amino | pb53 DLR aa | MAAMAERPFQCRICMRNFSQSGDLTRHIRTHTG |
| NO. 184 | acids | | EKPFACDICGRKFARSDNLSEHTKIHTGSQKPF |
| | | | QCRICMRNFSDRSALSEHIRTHTGEKPFACDIC |
| | | | GRKFARSSALSEHTKIHTGSQKPFQCRICMRNF |
| | | | SRSSHLTRHIRTHTGEKPFACDICGRKFARSDA |
| | | | LTRHTKIHTGSQKPFQCRICMRNFSRSDALSEH |
| | | | IRTHTGLRGSNSGDPRRHSLGGSRKPDLIAYKN |
| | | | FDLLVIVLKP* |
| SEQ ID | DNA | pb54 full length | GACTCTTCGCGATGTACGGGCCAGATATACGCG |
| NO. 185 | | | TTGACATTGATTATTGACTAGTTATTAATAGTA |
| | | | ATCAATTACGGGGTCATTAGTTCATAGCCCATA |
| | | | TATGGAGTTCCGCGTTACATAACTTACGGTAAA |
| | | | TGGCCCGCCTGGCTGACCGCCCAACGACCCCCG |
| | | | CCCATTGACGTCAATAATGACGTATGTTCCCAT |
| | | | AGTAACGCCAATAGGGACTTTCCATTGACGTCA |
| | | | ATGGGTGGACTATTTACGGTAAACTGCCCACTT |
| | | | GGCAGTACATCAAGTGTATCATATGCCAAGTAC |
| | | | GCCCCCTATTGACGTCAATGACGGTAAATGGCC |
| | | | CGCCTGGCATTATGCCCAGTACATGACCTTATG |
| | | | GGACTTTCCTACTTGGCAGTACATCTACGTATT |
| | | | AGTCATCGCTATTACCATGGTGATGCGGTTTTG |
| | | | GCAGTACATCAATGGGCGTGGATAGCGGTTTGA |
| | | | CTCACGGGGATTTCCAAGTCTCCACCCCATTGA |
| | | | CGTCAATGGGAGTTTGTTTTGGCACCAAAATCA |
| | | | ACGGGACTTTCCAAAATGTCGTAACAACTCCGC |
| | | | CCCATTGACGCAAATGGGCGGTAGGCGTGTACG |
| | | | GTGGGAGGTCTATATAAGCAGAGCTCTCTGGCT |
| | | | AACTAGAGAACCCACTGCTTACTGGCTTATCGA |
| | | | AATTAATACGACTCACTATAGGGAGACCCAAGC |
| | | | TGGCTAGCGTTTAAACTTAAGCTTATGGCGGCG |
| | | | ATGGCCGAGCGGCCCTTCCAGTGCAGGATCTGT |
| | | | ATGCGCAACTTTTCTCAGTCCGGCCACCTGACC |
| | | | CGGCACATCAGAACCCATACAGGCGAAAAGCCT |
| | | | TTCGCCTGCGACATTTGTGGGAGAAAATTTGCT |
| | | | CGGTCCGACGCCCTGACCCGGCATACCAAGATC |
| | | | CACACCGGCTCTCAGAAACCATTCCAGTGCCGC |
| | | | ATTTGTATGCGGAATTTTTCCACCTCCGGCGAC |
| | | | CTGTCCGAGCATATCCGCACTCACACCGGAGAG |
| | | | AAGCCCTTTGCTTGCGACATTTGTGGCAGGAAA |
| | | | TTTGCTCGGTCCTCCGACCTGACCCGGCACACT |
| | | | AAGATCCATACTGGGTCACAGAAACCTTTCCAG |
| | | | TGCCGGATTTGTATGAGAAACTTTAGCCGGTCC |
| | | | GACCACCTGTCCCAGCACATCAGAACCCATACA |
| | | | GGCGAAAAGCCTTTCGCCTGCGACATTTGTGGG |
| | | | AGAAAATTTGCTGACCGGTCCGACCTGACCCGG |
| | | | CATACCAAGATCCACACCGGCTCTCAGAAACCA |
| | | | TTCCAGTGCCGCATTTGTATGCGGAATTTTTCC |
| | | | CGGTCCGACGCCCTGTCCGAGCACATCAGAACA |
| | | | CATACTGGGCTGAGAGGATCCAATTCTGGTGAT |
| | | | CCTCGGAGACACAGTCTGGGCGGTTCTCGTAAA |
| | | | CCCGATCTGATTGCCTATAAAAACTTTGATCTG |
| | | | CTGGTCATTGTTCTTAAGCCTTGAGCGGCCGCT |
| | | | CGAGTCTAGAGGGCCCGTTTAAACCCGCTGATC |
| | | | AGCCTCGACTGTGCCTTCTAGTTGCCAGCCATC |
| | | | TGTTGTTTGCCCCTCCCCCGTGCCTTCCTTGAC |
| | | | CCTGGAAGGTGCCACTCCCACTGTCCTTTCCTA |
| | | | ATAAAATGAGGAAATTGCATCGCATTGTCTGAG |
| | | | TAGGTGTCATTCTATTCTGGGGGGTGGGGTGGG |
| | | | GCAGGACAGCAAGGGGGAGGATTGGGAAGACAA |
| | | | TAGCAGGCATGCTGGGGATGCGGTGGGCTCTAT |
| | | | GGCTTCTACTGGGCGGTTTTATGGACAGCAAGC |
| | | | GAACCGGAATTGCCAGCTGGGGCGCCCTCTGGT |
| | | | AAGGTTGGGAAGCCCTGCAAAGTAAACTGGATG |
| | | | GCTTTCTCGCCGCCAAGGATCTGATGGCGCAGG |
| | | | GGATCAAGCTCTGATCAAGAGACAGGATGAGGA |
| | | | TCGTTTCGCATGATTGAACAAGATGGATTGCAC |
| | | | GCAGGTTCTCCGGCCGCTTGGGTGGAGAGGCTA |
| | | | TTCGGCTATGACTGGGCACAACAGACAATCGGC |
| | | | TGCTCTGATGCCGCCGTGTTCCGGCTGTCAGCG |
| | | | CAGGGGCGCCCGGTTCTTTTTGTCAAGACCGAC |
| | | | CTGTCCGGTGCCCTGAATGAACTGCAAGACGAG |
| | | | GCAGCGCGGCTATCGTGGCTGGCCACGACGGGC |
| | | | GTTCCTTGCGCAGCTGTGCTCGACGTTGTCACT |
| | | | GAAGCGGGAAGGGACTGGCTGCTATTGGGCGAA |
| | | | GTGCCGGGGCAGGATCTCCTGTCATCTCACCTT |
| | | | GCTCCTGCCGAGAAAGTATCCATCATGGCTGAT |
| | | | GCAATGCGGCGGCTGCATACGCTTGATCCGGCT |
| | | | ACCTGCCCATTCGACCACCAAGCGAAACATCGC |
| | | | ATCGAGCGAGCACGTACTCGGATGGAAGCCGGT |
| | | | CTTGTCGATCAGGATGATCTGGACGAAGAGCAT |
| | | | CAGGGGCTCGCGCCAGCCGAACTGTTCGCCAGG |
| | | | CTCAAGGCGAGCATGCCCGACGGCGAGGATCTC |
| | | | GTCGTGACCCATGGCGATGCCTGCTTGCCGAAT |
| | | | ATCATGGTGGAAAATGGCCGCTTTTCTGGATTC |
| | | | ATCGACTGTGGCCGGCTGGGTGTGGCGGACCGC |
| | | | TATCAGGACATAGCGTTGGCTACCCGTGATATT |
| | | | GCTGAAGAGCTTGGCGGCGAATGGGCTGACCGC |
| | | | TTCCTCGTGCTTTACGGTATCGCCGCTCCCGAT |
| | | | TCGCAGCGCATCGCCTTCTATCGCCTTCTTGAC |
| | | | GAGTTCTTCTGAATTATTAACGCTTACAATTTC |
| | | | CTGATGCGGTATTTTCTCCTTACGCATCTGTGC |
| | | | GGTATTTCACACCGCATACAGGTGGCACTTTTC |
| | | | GGGGAAATGTGCGCGGAACCCCTATTTGTTTAT |
| | | | TTTTCTAAATACATTCAAATATGTATCCGCTCA |
| | | | TGAGACAATAACCCTGATAAATGCTTCAATAAT |
| | | | AGCACGTGCTAAAACTTCATTTTTAATTTAAAA |
| | | | GGATCTAGGTGAAGATCCTTTTTGATAATCTCA |
| | | | TGACCAAAATCCCTTAACGTGAGTTTTCGTTCC |
| | | | ACTGAGCGTCAGACCCCGTAGAAAAGATCAAAG |
| | | | GATCTTCTTGAGATCCTTTTTTTCTGCGCGTAA |
| | | | TCTGCTGCTTGCAAACAAAAAAACCACCGCTAC |
| | | | CAGCGGTGGTTTGTTTGCCGGATCAAGAGCTAC |
| | | | CAACTCTTTTTCCGAAGGTAACTGGCTTCAGCA |
| | | | GAGCGCAGATACCAAATACTGTCCTTCTAGTGT |
| | | | AGCCGTAGTTAGGCCACCACTTCAAGAACTCTG |
| | | | TAGCACCGCCTACATACCTCGCTCTGCTAATCC |
| | | | TGTTACCAGTGGCTGCTGCCAGTGGCGATAAGT |
| | | | CGTGTCTTACCGGGTTGGACTCAAGACGATAGT |
| | | | TACCGGATAAGGCGCAGCGGTCGGGCTGAACGG |
| | | | GGGGTTCGTGCACACAGCCCAGCTTGGAGCGAA |
| | | | CGACCTACACCGAACTGAGATACCTACAGCGTG |
| | | | AGCTATGAGAAAGCGCCACGCTTCCCGAAGGGA |
| | | | GAAAGGCGGACAGGTATCCGGTAAGCGGCAGGG |
| | | | TCGGAACAGGAGAGCGCACGAGGGAGCTTCCAG |
| | | | GGGGAAACGCCTGGTATCTTTATAGTCCTGTCG |
| | | | GGTTTCGCCACCTCTGACTTGAGCGTCGATTTT |
| | | | TGTGATGCTCGTCAGGGGGGCGGAGCCTATGGA |
| | | | AAAACGCCAGCAACGCGGCCTTTTTACGGTTCC |
| | | | TGGGCTTTTGCTGGCCTTTTGCTCACATGTTCT |
| | | | T |
| SEQ ID | DNA | pb54 cDNA | ATGGCGGCGATGGCCGAGCGGCCCTTCCAGTGC |
| NO. 186 | | | AGGATCTGTATGCGCAACTTTTCTCAGTCCGGC |
| | | | CACCTGACCCGGCACATCAGAACCCATACAGGC |
| | | | GAAAAGCCTTTCGCCTGCGACATTTGTGGGAGA |
| | | | AAATTTGCTCGGTCCGACGCCCTGACCCGGCAT |
| | | | ACCAAGATCCACACCGGCTCTCAGAAACCATTC |
| | | | CAGTGCCGCATTTGTATGCGGAATTTTTCCACC |
| | | | TCCGGCGACCTGTCCGAGCATATCCGCACTCAC |
| | | | ACCGGAGAGAAGCCCTTTGCTTGCGACATTTGT |
| | | | GGCAGGAAATTTGCTCGGTCCTCCGACCTGACC |
| | | | CGGCACACTAAGATCCATACTGGGTCACAGAAA |
| | | | CCTTTCCAGTGCCGGATTTGTATGAGAAACTTT |
| | | | AGCCGGTCCGACCACCTGTCCCAGCACATCAGA |
| | | | ACCCATACAGGCGAAAAGCCTTTCGCCTGCGAC |
| | | | ATTTGTGGGAGAAAATTTGCTGACCGGTCCGAC |
| | | | CTGACCCGGCATACCAAGATCCACACCGGCTCT |
| | | | CAGAAACCATTCCAGTGCCGCATTTGTATGCGG |
| | | | AATTTTTCCCGGTCCGACGCCCTGTCCGAGCAC |
| | | | ATCAGAACACATACTGGGCTGAGAGGATCCAAT |
| | | | TCTGGTGATCCTCGGAGACACAGTCTGGGCGGT |
| | | | TCTCGTAAACCCGATCTGATTGCCTATAAAAAC |
| | | | TTTGATCTGCTGGTCATTGTTCTTAAGCCTTGA |
| SEQ ID | Amino | pb54 DLR Amino | MAAMAERPFQCRICMRNFSQSGHLTRHIRTHTG |
| NO. 187 | acids | acids | EKPFACDICGRKFARSDALTRHTKIHTGSQKPF |
| | | | QCRICMRNESTSGDLSEHIRTHTGEKPFACDIC |
| | | | GRKFARSSDLTRHTKIHTGSQKPFQCRICMRNF |
| | | | SRSDHLSQHIRTHTGEKPFACDICGRKFADRSD |
| | | | LTRHTKIHTGSQKPFQCRICMRNFSRSDALSEH |
| | | | IRTHTGLRGSNSGDPRRHSLGGSRKPDLIAYKN |
| | | | FDLLVIVLKP* |
| SEQ ID | DNA | pb52, and pb54 D | 5′-CTG-GTG-GGG-CTG-CTC-CAG-GCA |
| NO. 188 | | recognition | |
| | | sequence | |
| SEQ ID | DNA | pb53 and pb54 R | 5′-CTG-GCC-AGG-GCG-CCT-GTG-GGA |
| NO. 189 | | recognition | |
| | | sequence | |
| SEQ ID | DNA | Pop102 PDCD1 | TTTCCCTTCCGCTCACCTCCGCCTGAGCAGTGG |
| NO. 190 | | ODN F2 | AGAAGGCGGCACTCTGGTGGGGCTGCTCCAGGC |
| | | | ATG aat tCA |
| | | | tGATCCCACAGGCGCCCTGGCCAGTCGTCTGGG |
| | | | CGGTGCTACAACTGGGCTGGCGGCCAGGATGGT |
| | | | TCTTAGGT |
| SEQ ID | DNA | Pop90 PDCD1 F (1) | GCCTGAGCAGTGGAGAAGG |
| NO. 191 | | in | |
| SEQ ID | DNA | Pop91 PDCD1 R | GGACTGAGGGTGGAAGGTC |
| NO. 192 | | (1) out | |
| SEQ ID | DNA | Ref seq for a | ACTCTTAGACATAACACACCAGGGTCAATACAA |
| NO. 193 | | GATAA box region | CTTTGAAGCTAGTCTAGTGCAAGCTAACAGTTG |
| | | in human BCL11 | CTTTTATCACAGGCTCCAGGAAGGGTTTGGCCT |
| | | | CTGATTAGGGTGGGGGCGTGGGTGGGGTAGAAG |
| | | | AGGACTGGCAGACCTCTCCATCGGTGGCCGTTT |
| | | | GCCCAGGGGGGCCTCTTTCGGAAGGCTCTCTT |
| SEQ ID | DNA | pb64 full length | GACTCTTCGCGATGTACGGGCCAGATATACGCG |
| NO. 194 | | | TTGACATTGATTATTGACTAGTTATTAATAGTA |
| | | | ATCAATTACGGGGTCATTAGTTCATAGCCCATA |
| | | | TATGGAGTTCCGCGTTACATAACTTACGGTAAA |
| | | | TGGCCCGCCTGGCTGACCGCCCAACGACCCCCG |
| | | | CCCATTGACGTCAATAATGACGTATGTTCCCAT |
| | | | AGTAACGCCAATAGGGACTTTCCATTGACGTCA |
| | | | ATGGGTGGACTATTTACGGTAAACTGCCCACTT |
| | | | GGCAGTACATCAAGTGTATCATATGCCAAGTAC |
| | | | GCCCCCTATTGACGTCAATGACGGTAAATGGCC |
| | | | CGCCTGGCATTATGCCCAGTACATGACCTTATG |
| | | | GGACTTTCCTACTTGGCAGTACATCTACGTATT |
| | | | AGTCATCGCTATTACCATGGTGATGCGGTTTTG |
| | | | GCAGTACATCAATGGGCGTGGATAGCGGTTTGA |
| | | | CTCACGGGGATTTCCAAGTCTCCACCCCATTGA |
| | | | CGTCAATGGGAGTTTGTTTTGGCACCAAAATCA |
| | | | ACGGGACTTTCCAAAATGTCGTAACAACTCCGC |
| | | | CCCATTGACGCAAATGGGCGGTAGGCGTGTACG |
| | | | GTGGGAGGTCTATATAAGCAGAGCTCTCTGGCT |
| | | | AACTAGAGAACCCACTGCTTACTGGCTTATCGA |
| | | | AATTAATACGACTCACTATAGGGAGACCCAAGC |
| | | | TGGCTAGCGTTTAAACTTAAGCTTATGGCGGCG |
| | | | ATGGCCGAGCGGCCCTTCGCCTGCGACATTTGT |
| | | | GGGAGAAAATTTGCTGATCAGTCCGGCAACCTG |
| | | | ACCCGGCATACCAAGATCCACACCGGCTCTCAG |
| | | | AAACCATTCCAGTGCAGGATCTGTATGCGCAAC |
| | | | TTTTCTCGGTCCGACAACCTGTCCGAGCACATC |
| | | | AGAACCCATACAGGCGAAAAGCCTTTTGCTTGC |
| | | | GACATTTGTGGCAGGAAATTTGCTGACTCCTCC |
| | | | GCCCTGTCCCAGCACACTAAGATCCATACTGGG |
| | | | TCACAGAAACCTTTCCAGTGCCGCATTTGTATG |
| | | | CGGAATTTTTCCCAGTCCGGCTCCCTGTCCCAG |
| | | | CATATCCGCACTCACACCGGAGAGAAGCCCTTT |
| | | | GCATGCGACATTTGTGGACGGAAATTTGCTGAC |
| | | | CGGTCCCACCTGACCCGGCATACCAAGATTCAC |
| | | | ACTGGGTCTCAGAAACCTTTCCAGTGCAGGATT |
| | | | TGTATGAGAAATTTTTCCCAGTCCGGCGACCTG |
| | | | TCCGAGCACATCAGAACCCATACAGGCGAAAAG |
| | | | CCTTTTGCTTGCGACATTTGTGGCAGGAAATTT |
| | | | GCTCGGTCCTCCGCCCTGACCCGGCACACTAAG |
| | | | ATCCATACTGGGTCACAGAAACCTTTCCAGTGC |
| | | | CGCATTTGTATGCGGAATTTTTCCCGGTCCGAC |
| | | | TCCCTGTCCCAGCACATCAGAACACATACTGGG |
| | | | CTGAGAGGATCCAATTCTGGTGATCCTCGGAGA |
| | | | CACAGTCTGGGCGGTTCTCGTAAACCCGATCTG |
| | | | ATTGCCTATAAAAACTTTGATCTGCTGGTCATT |
| | | | GTTCTTAAGCCTTGAGCGGCCGCTCGAGTCTAG |
| | | | AGGGCCCGTTTAAACCCGCTGATCAGCCTCGAC |
| | | | TGTGCCTTCTAGTTGCCAGCCATCTGTTGTTTG |
| | | | CCCCTCCCCCGTGCCTTCCTTGACCCTGGAAGG |
| | | | TGCCACTCCCACTGTCCTTTCCTAATAAAATGA |
| | | | GGAAATTGCATCGCATTGTCTGAGTAGGTGTCA |
| | | | TTCTATTCTGGGGGGTGGGGTGGGGCAGGACAG |
| | | | CAAGGGGGAGGATTGGGAAGACAATAGCAGGCA |
| | | | TGCTGGGGATGCGGTGGGCTCTATGGCTTCTAC |
| | | | TGGGCGGTTTTATGGACAGCAAGCGAACCGGAA |
| | | | TTGCCAGCTGGGGCGCCCTCTGGTAAGGTTGGG |
| | | | AAGCCCTGCAAAGTAAACTGGATGGCTTTCTCG |
| | | | CCGCCAAGGATCTGATGGCGCAGGGGATCAAGC |
| | | | TCTGATCAAGAGACAGGATGAGGATCGTTTCGC |
| | | | ATGATTGAACAAGATGGATTGCACGCAGGTTCT |
| | | | CCGGCCGCTTGGGTGGAGAGGCTATTCGGCTAT |
| | | | GACTGGGCACAACAGACAATCGGCTGCTCTGAT |
| | | | GCCGCCGTGTTCCGGCTGTCAGCGCAGGGGCGC |
| | | | CCGGTTCTTTTTGTCAAGACCGACCTGTCCGGT |
| | | | GCCCTGAATGAACTGCAAGACGAGGCAGCGCGG |
| | | | CTATCGTGGCTGGCCACGACGGGCGTTCCTTGC |
| | | | GCAGCTGTGCTCGACGTTGTCACTGAAGCGGGA |
| | | | AGGGACTGGCTGCTATTGGGCGAAGTGCCGGGG |
| | | | CAGGATCTCCTGTCATCTCACCTTGCTCCTGCC |
| | | | GAGAAAGTATCCATCATGGCTGATGCAATGCGG |
| | | | CGGCTGCATACGCTTGATCCGGCTACCTGCCCA |
| | | | TTCGACCACCAAGCGAAACATCGCATCGAGCGA |
| | | | GCACGTACTCGGATGGAAGCCGGTCTTGTCGAT |
| | | | CAGGATGATCTGGACGAAGAGCATCAGGGGCTC |
| | | | GCGCCAGCCGAACTGTTCGCCAGGCTCAAGGCG |
| | | | AGCATGCCCGACGGCGAGGATCTCGTCGTGACC |
| | | | CATGGCGATGCCTGCTTGCCGAATATCATGGTG |
| | | | GAAAATGGCCGCTTTTCTGGATTCATCGACTGT |
| | | | GGCCGGCTGGGTGTGGCGGACCGCTATCAGGAC |
| | | | ATAGCGTTGGCTACCCGTGATATTGCTGAAGAG |
| | | | CTTGGCGGCGAATGGGCTGACCGCTTCCTCGTG |
| | | | CTTTACGGTATCGCCGCTCCCGATTCGCAGCGC |
| | | | ATCGCCTTCTATCGCCTTCTTGACGAGTTCTTC |
| | | | TGAATTATTAACGCTTACAATTTCCTGATGCGG |
| | | | TATTTTCTCCTTACGCATCTGTGCGGTATTTCA |
| | | | CACCGCATACAGGTGGCACTTTTCGGGGAAATG |
| | | | TGCGCGGAACCCCTATTTGTTTATTTTTCTAAA |
| | | | TACATTCAAATATGTATCCGCTCATGAGACAAT |
| | | | AACCCTGATAAATGCTTCAATAATAGCACGTGC |
| | | | TAAAACTTCATTTTTAATTTAAAAGGATCTAGG |
| | | | TGAAGATCCTTTTTGATAATCTCATGACCAAAA |
| | | | TCCCTTAACGTGAGTTTTCGTTCCACTGAGCGT |
| | | | CAGACCCCGTAGAAAAGATCAAAGGATCTTCTT |
| | | | GAGATCCTTTTTTTCTGCGCGTAATCTGCTGCT |
| | | | TGCAAACAAAAAAACCACCGCTACCAGCGGTGG |
| | | | TTTGTTTGCCGGATCAAGAGCTACCAACTCTTT |
| | | | TTCCGAAGGTAACTGGCTTCAGCAGAGCGCAGA |
| | | | TACCAAATACTGTCCTTCTAGTGTAGCCGTAGT |
| | | | TAGGCCACCACTTCAAGAACTCTGTAGCACCGC |
| | | | CTACATACCTCGCTCTGCTAATCCTGTTACCAG |
| | | | TGGCTGCTGCCAGTGGCGATAAGTCGTGTCTTA |
| | | | CCGGGTTGGACTCAAGACGATAGTTACCGGATA |
| | | | AGGCGCAGCGGTCGGGCTGAACGGGGGGTTCGT |
| | | | GCACACAGCCCAGCTTGGAGCGAACGACCTACA |
| | | | CCGAACTGAGATACCTACAGCGTGAGCTATGAG |
| | | | AAAGCGCCACGCTTCCCGAAGGGAGAAAGGCGG |
| | | | ACAGGTATCCGGTAAGCGGCAGGGTCGGAACAG |
| | | | GAGAGCGCACGAGGGAGCTTCCAGGGGGAAACG |
| | | | CCTGGTATCTTTATAGTCCTGTCGGGTTTCGCC |
| | | | ACCTCTGACTTGAGCGTCGATTTTTGTGATGCT |
| | | | CGTCAGGGGGGCGGAGCCTATGGAAAAACGCCA |
| | | | GCAACGCGGCCTTTTTACGGTTCCTGGGCTTTT |
| | | | GCTGGCCTTTTGCTCACATGTTCTT |
| SEQ ID | DNA | pb64 cDNA | ATGGCGGCGATGGCCGAGCGGCCCTTCGCCTGC |
| NO. 195 | | | GACATTTGTGGGAGAAAATTTGCTGATCAGTCC |
| | | | GGCAACCTGACCCGGCATACCAAGATCCACACC |
| | | | GGCTCTCAGAAACCATTCCAGTGCAGGATCTGT |
| | | | ATGCGCAACTTTTCTCGGTCCGACAACCTGTCC |
| | | | GAGCACATCAGAACCCATACAGGCGAAAAGCCT |
| | | | TTTGCTTGCGACATTTGTGGCAGGAAATTTGCT |
| | | | GACTCCTCCGCCCTGTCCCAGCACACTAAGATC |
| | | | CATACTGGGTCACAGAAACCTTTCCAGTGCCGC |
| | | | ATTTGTATGCGGAATTTTTCCCAGTCCGGCTCC |
| | | | CTGTCCCAGCATATCCGCACTCACACCGGAGAG |
| | | | AAGCCCTTTGCATGCGACATTTGTGGACGGAAA |
| | | | TTTGCTGACCGGTCCCACCTGACCCGGCATACC |
| | | | AAGATTCACACTGGGTCTCAGAAACCTTTCCAG |
| | | | TGCAGGATTTGTATGAGAAATTTTTCCCAGTCC |
| | | | GGCGACCTGTCCGAGCACATCAGAACCCATACA |
| | | | GGCGAAAAGCCTTTTGCTTGCGACATTTGTGGC |
| | | | AGGAAATTTGCTCGGTCCTCCGCCCTGACCCGG |
| | | | CACACTAAGATCCATACTGGGTCACAGAAACCT |
| | | | TTCCAGTGCCGCATTTGTATGCGGAATTTTTCC |
| | | | CGGTCCGACTCCCTGTCCCAGCACATCAGAACA |
| | | | CATACTGGGCTGAGAGGATCCAATTCTGGTGAT |
| | | | CCTCGGAGACACAGTCTGGGCGGTTCTCGTAAA |
| | | | CCCGATCTGATTGCCTATAAAAACTTTGATCTG |
| | | | CTGGTCATTGTTCTTAAGCCTTGA |
| SEQ ID NO. | DNA | pb64 DLR amino | MAAMAERPFACDICGRKFADQSGNLTRHTKIHT |
| 196 | | acids | GSQKPFQCRICMRNFSRSDNLSEHIRTHTGEKP |
| | | | FACDICGRKFADSSALSQHTKIHTGSQKPFQCR |
| | | | ICMRNFSQSGSLSQHIRTHTGEKPFACDICGRK |
| | | | FADRSHLTRHTKIHTGSQKPFQCRICMRNFSQS |
| | | | GDLSEHIRTHTGEKPFACDICGRKFARSSALTR |
| | | | HTKIHTGSQKPFQCRICMRNFSRSDSLSQHIRT |
| | | | HTGLRGSNSGDPRRHSLGGSRKPDLIAYKNEDL |
| | | | LVIVLKP* |
| SEQ ID NO. | DNA | pb64 D recognition | ATG-GTG-CCA-GGC-ATA-ATC-CAG-GAA |
| 197 | | sequence | |
| SEQ ID NO. | DNA | Pop 104 CFTR ODN | GAATTTCATTCTGTTCTCAGTTTTCCTGGATTA |
| 198 | | F | TGCCTGGCACCATTAAAGAAAATATCATATGTG |
| | | | GTGTTTCCTATGATGAATATAGATACAGAAGCG |
| | | | TCATCAAAGCATGCCAACTAGAAGAGGTAAG |
| SEQ ID NO. | DNA | Pop105 CFTR F | TGGAGCCTTCAGAGGGTAAA |
| 199 | | external | |
| SEQ ID NO. | DNA | Pop 106 CFTR R | AGTTGGCATGCTTTGATGAC |
| 200 | | internal | |
| SEQ ID NO. | DNA | Pop 107 CFTR wt | CCATTAAAGAAAATATCATCTTTGGTGTTTCC |
| 201 | | CTT probe F Hex | |
| SEQ ID NO. | DNA | Pop108 CFTR Rpr | AAATATCATATGTGGTGTTTCCTATG |
| 202 | | ATG probe F Fam | |
| SEQ ID NO. | RNA | Pop98-crRNA | mG*mG*CGCAGGCCCGGCUGGGCGGUUUUAGAG |
| 203 | | (2899-2918 | CUAUG*mC*mU |
| | | ApoE112) | |
| SEQ ID NO. | DNA | POP98-crRNA | GGCGCAGGCCCGGCTGGGCG |
| 204 | | guide RNA binding | |
| | | site | |
| SEQ ID NO. | DNA | crRNa (ApoE 1112 | CCTGGTGCAGTACCGCGGCG |
| 205 | | crRNA2) binding | |
| | | site | |
| SEQ ID NO. | DNA | pb73: pSpCas9d | GAGGGCCTATTTCCCATGATTCCTTCATATTTG |
| 206 | | | CATATACGATACAAGGCTGTTAGAGAGATAATT |
| | | | GGAATTAATTTGACTGTAAACACAAAGATATTA |
| | | | GTACAAAATACGTGACGTAGAAAGTAATAATTT |
| | | | CTTGGGTAGTTTGCAGTTTTAAAATTATGTTTT |
| | | | AAAATGGACTATCATATGCTTACCGTAACTTGA |
| | | | AAGTATTTCGATTTCTTGGCTTTATATATCTTG |
| | | | TGGAAAGGACGAAACACCGGGTCTTCGAGAAGA |
| | | | CCTGTTTTAGAGCTAGAAATAGCAAGTTAAAAT |
| | | | AAGGCTAGTCCGTTATCAACTTGAAAAAGTGGC |
| | | | ACCGAGTCGGTGCTTTTTTGTTTTAGAGCTAGA |
| | | | AATAGCAAGTTAAAATAAGGCTAGTCCGTTTTT |
| | | | AGCGCGTGCGCCAATTCTGCAGACAAATGGCTC |
| | | | TAGAGGTACCCGTTACATAACTTACGGTAAATG |
| | | | GCCCGCCTGGCTGACCGCCCAACGACCCCCGCC |
| | | | CATTGACGTCAATAGTAACGCCAATAGGGACTT |
| | | | TCCATTGACGTCAATGGGTGGAGTATTTACGGT |
| | | | AAACTGCCCACTTGGCAGTACATCAAGTGTATC |
| | | | ATATGCCAAGTACGCCCCCTATTGACGTCAATG |
| | | | ACGGTAAATGGCCCGCCTGGCATTGTGCCCAGT |
| | | | ACATGACCTTATGGGACTTTCCTACTTGGCAGT |
| | | | ACATCTACGTATTAGTCATCGCTATTACCATGG |
| | | | TCGAGGTGAGCCCCACGTTCTGCTTCACTCTCC |
| | | | CCATCTCCCCCCCCTCCCCACCCCCAATTTTGT |
| | | | ATTTATTTATTTTTTAATTATTTTGTGCAGCGA |
| | | | TGGGGGCGGGGGGGGGGGGGGGGCGCGCGCCAG |
| | | | GCGGGGCGGGGCGGGGCGAGGGGCGGGGCGGGG |
| | | | CGAGGCGGAGAGGTGCGGCGGCAGCCAATCAGA |
| | | | GCGGCGCGCTCCGAAAGTTTCCTTTTATGGCGA |
| | | | GGCGGCGGCGGCGGCGGCCCTATAAAAAGCGAA |
| | | | GCGCGCGGCGGGCGGGAGTCGCTGCGACGCTGC |
| | | | CTTCGCCCCGTGCCCCGCTCCGCCGCCGCCTCG |
| | | | CGCCGCCCGCCCCGGCTCTGACTGACCGCGTTA |
| | | | CTCCCACAGGTGAGCGGGCGGGACGGCCCTTCT |
| | | | CCTCCGGGCTGTAATTAGCTGAGCAAGAGGTAA |
| | | | GGGTTTAAGGGATGGTTGGTTGGTGGGGTATTA |
| | | | ATGTTTAATTACCTGGAGCACCTGCCTGAAATC |
| | | | ACTTTTTTTCAGGTTGGACCGGTGCCACCATGG |
| | | | ACTATAAGGACCACGACGGAGACTACAAGGATC |
| | | | ATGATATTGATTACAAAGACGATGACGATAAGA |
| | | | TGGCCCCAAAGAAGAAGCGGAAGGTCGGTATCC |
| | | | ACGGAGTCCCAGCAGCCGACAAGAAGTACAGCA |
| | | | TCGGCCTGGCCATCGGCACCAACTCTGTGGGCT |
| | | | GGGCCGTGATCACCGACGAGTACAAGGTGCCCA |
| | | | GCAAGAAATTCAAGGTGCTGGGCAACACCGACC |
| | | | GGCACAGCATCAAGAAGAACCTGATCGGAGCCC |
| | | | TGCTGTTCGACAGCGGCGAAACAGCCGAGGCCA |
| | | | CCCGGCTGAAGAGAACCGCCAGAAGAAGATACA |
| | | | CCAGACGGAAGAACCGGATCTGCTATCTGCAAG |
| | | | AGATCTTCAGCAACGAGATGGCCAAGGTGGACG |
| | | | ACAGCTTCTTCCACAGACTGGAAGAGTCCTTCC |
| | | | TGGTGGAAGAGGATAAGAAGCACGAGCGGCACC |
| | | | CCATCTTCGGCAACATCGTGGACGAGGTGGCCT |
| | | | ACCACGAGAAGTACCCCACCATCTACCACCTGA |
| | | | GAAAGAAACTGGTGGACAGCACCGACAAGGCCG |
| | | | ACCTGCGGCTGATCTATCTGGCCCTGGCCCACA |
| | | | TGATCAAGTTCCGGGGCCACTTCCTGATCGAGG |
| | | | GCGACCTGAACCCCGACAACAGCGACGTGGACA |
| | | | AGCTGTTCATCCAGCTGGTGCAGACCTACAACC |
| | | | AGCTGTTCGAGGAAAACCCCATCAACGCCAGCG |
| | | | GCGTGGACGCCAAGGCCATCCTGTCTGCCAGAC |
| | | | TGAGCAAGAGCAGACGGCTGGAAAATCTGATCG |
| | | | CCCAGCTGCCCGGCGAGAAGAAGAATGGCCTGT |
| | | | TCGGCAACCTGATTGCCCTGAGCCTGGGCCTGA |
| | | | CCCCCAACTTCAAGAGCAACTTCGACCTGGCCG |
| | | | AGGATGCCAAACTGCAGCTGAGCAAGGACACCT |
| | | | ACGACGACGACCTGGACAACCTGCTGGCCCAGA |
| | | | TCGGCGACCAGTACGCCGACCTGTTTCTGGCCG |
| | | | CCAAGAACCTGTCCGACGCCATCCTGCTGAGCG |
| | | | ACATCCTGAGAGTGAACACCGAGATCACCAAGG |
| | | | CCCCCCTGAGCGCCTCTATGATCAAGAGATACG |
| | | | ACGAGCACCACCAGGACCTGACCCTGCTGAAAG |
| | | | CTCTCGTGCGGCAGCAGCTGCCTGAGAAGTACA |
| | | | AAGAGATTTTCTTCGACCAGAGCAAGAACGGCT |
| | | | ACGCCGGCTACATTGACGGCGGAGCCAGCCAGG |
| | | | AAGAGTTCTACAAGTTCATCAAGCCCATCCTGG |
| | | | AAAAGATGGACGGCACCGAGGAACTGCTCGTGA |
| | | | AGCTGAACAGAGAGGACCTGCTGCGGAAGCAGC |
| | | | GGACCTTCGACAACGGCAGCATCCCCCACCAGA |
| | | | TCCACCTGGGAGAGCTGCACGCCATTCTGCGGC |
| | | | GGCAGGAAGATTTTTACCCATTCCTGAAGGACA |
| | | | ACCGGGAAAAGATCGAGAAGATCCTGACCTTCC |
| | | | GCATCCCCTACTACGTGGGCCCTCTGGCCAGGG |
| | | | GAAACAGCAGATTCGCCTGGATGACCAGAAAGA |
| | | | GCGAGGAAACCATCACCCCCTGGAACTTCGAGG |
| | | | AAGTGGTGGACAAGGGCGCTTCCGCCCAGAGCT |
| | | | TCATCGAGCGGATGACCAACTTCGATAAGAACC |
| | | | TGCCCAACGAGAAGGTGCTGCCCAAGCACAGCC |
| | | | TGCTGTACGAGTACTTCACCGTGTATAACGAGC |
| | | | TGACCAAAGTGAAATACGTGACCGAGGGAATGA |
| | | | GAAAGCCCGCCTTCCTGAGCGGCGAGCAGAAAA |
| | | | AGGCCATCGTGGACCTGCTGTTCAAGACCAACC |
| | | | GGAAAGTGACCGTGAAGCAGCTGAAAGAGGACT |
| | | | ACTTCAAGAAAATCGAGTGCTTCGACTCCGTGG |
| | | | AAATCTCCGGCGTGGAAGATCGGTTCAACGCCT |
| | | | CCCTGGGCACATACCACGATCTGCTGAAAATTA |
| | | | TCAAGGACAAGGACTTCCTGGACAATGAGGAAA |
| | | | ACGAGGACATTCTGGAAGATATCGTGCTGACCC |
| | | | TGACACTGTTTGAGGACAGAGAGATGATCGAGG |
| | | | AACGGCTGAAAACCTATGCCCACCTGTTCGACG |
| | | | ACAAAGTGATGAAGCAGCTGAAGCGGCGGAGAT |
| | | | ACACCGGCTGGGGCAGGCTGAGCCGGAAGCTGA |
| | | | TCAACGGCATCCGGGACAAGCAGTCCGGCAAGA |
| | | | CAATCCTGGATTTCCTGAAGTCCGACGGCTTCG |
| | | | CCAACAGAAACTTCATGCAGCTGATCCACGACG |
| | | | ACAGCCTGACCTTTAAAGAGGACATCCAGAAAG |
| | | | CCCAGGTGTCCGGCCAGGGCGATAGCCTGCACG |
| | | | AGCACATTGCCAATCTGGCCGGCAGCCCCGCCA |
| | | | TTAAGAAGGGCATCCTGCAGACAGTGAAGGTGG |
| | | | TGGACGAGCTCGTGAAAGTGATGGGCCGGCACA |
| | | | AGCCCGAGAACATCGTGATCGAAATGGCCAGAG |
| | | | AGAACCAGACCACCCAGAAGGGACAGAAGAACA |
| | | | GCCGCGAGAGAATGAAGCGGATCGAAGAGGGCA |
| | | | TCAAAGAGCTGGGCAGCCAGATCCTGAAAGAAC |
| | | | ACCCCGTGGAAAACACCCAGCTGCAGAACGAGA |
| | | | AGCTGTACCTGTACTACCTGCAGAATGGGCGGG |
| | | | ATATGTACGTGGACCAGGAACTGGACATCAACC |
| | | | GGCTGTCCGACTACGATGTGGACGCCATCGTGC |
| | | | CTCAGAGCTTTCTGAAGGACGACTCCATCGACA |
| | | | ACAAGGTGCTGACCAGAAGCGACAAGAACCGGG |
| | | | GCAAGAGCGACAACGTGCCCTCCGAAGAGGTCG |
| | | | TGAAGAAGATGAAGAACTACTGGCGGCAGCTGC |
| | | | TGAACGCCAAGCTGATTACCCAGAGAAAGTTCG |
| | | | ACAATCTGACCAAGGCCGAGAGAGGCGGCCTGA |
| | | | GCGAACTGGATAAGGCCGGCTTCATCAAGAGAC |
| | | | AGCTGGTGGAAACCCGGCAGATCACAAAGCACG |
| | | | TGGCACAGATCCTGGACTCCCGGATGAACACTA |
| | | | AGTACGACGAGAATGACAAGCTGATCCGGGAAG |
| | | | TGAAAGTGATCACCCTGAAGTCCAAGCTGGTGT |
| | | | CCGATTTCCGGAAGGATTTCCAGTTTTACAAAG |
| | | | TGCGCGAGATCAACAACTACCACCACGCCCACG |
| | | | ACGCCTACCTGAACGCCGTCGTGGGAACCGCCC |
| | | | TGATCAAAAAGTACCCTAAGCTGGAAAGCGAGT |
| | | | TCGTGTACGGCGACTACAAGGTGTACGACGTGC |
| | | | GGAAGATGATCGCCAAGAGCGAGCAGGAAATCG |
| | | | GCAAGGCTACCGCCAAGTACTTCTTCTACAGCA |
| | | | ACATCATGAACTTTTTCAAGACCGAGATTACCC |
| | | | TGGCCAACGGCGAGATCCGGAAGCGGCCTCTGA |
| | | | TCGAGACAAACGGCGAAACCGGGGAGATCGTGT |
| | | | GGGATAAGGGCCGGGATTTTGCCACCGTGCGGA |
| | | | AAGTGCTGAGCATGCCCCAAGTGAATATCGTGA |
| | | | AAAAGACCGAGGTGCAGACAGGCGGCTTCAGCA |
| | | | AAGAGTCTATCCTGCCCAAGAGGAACAGCGATA |
| | | | AGCTGATCGCCAGAAAGAAGGACTGGGACCCTA |
| | | | AGAAGTACGGCGGCTTCGACAGCCCCACCGTGG |
| | | | CCTATTCTGTGCTGGTGGTGGCCAAAGTGGAAA |
| | | | AGGGCAAGTCCAAGAAACTGAAGAGTGTGAAAG |
| | | | AGCTGCTGGGGATCACCATCATGGAAAGAAGCA |
| | | | GCTTCGAGAAGAATCCCATCGACTTTCTGGAAG |
| | | | CCAAGGGCTACAAAGAAGTGAAAAAGGACCTGA |
| | | | TCATCAAGCTGCCTAAGTACTCCCTGTTCGAGC |
| | | | TGGAAAACGGCCGGAAGAGAATGCTGGCCTCTG |
| | | | CCGGCGAACTGCAGAAGGGAAACGAACTGGCCC |
| | | | TGCCCTCCAAATATGTGAACTTCCTGTACCTGG |
| | | | CCAGCCACTATGAGAAGCTGAAGGGCTCCCCCG |
| | | | AGGATAATGAGCAGAAACAGCTGTTTGTGGAAC |
| | | | AGCACAAGCACTACCTGGACGAGATCATCGAGC |
| | | | AGATCAGCGAGTTCTCCAAGAGAGTGATCCTGG |
| | | | CCGACGCTAATCTGGACAAAGTGCTGTCCGCCT |
| | | | ACAACAAGCACCGGGATAAGCCCATCAGAGAGC |
| | | | AGGCCGAGAATATCATCCACCTGTTTACCCTGA |
| | | | CCAATCTGGGAGCCCCTGCCGCCTTCAAGTACT |
| | | | TTGACACCACCATCGACCGGAAGAGGTACACCA |
| | | | GCACCAAAGAGGTGCTGGACGCCACCCTGATCC |
| | | | ACCAGAGCATCACCGGCCTGTACGAGACACGGA |
| | | | TCGACCTGTCTCAGCTGGGAGGCGACAAAAGGC |
| | | | CGGCGGCCACGAAAAAGGCCGGCCAGGCAAAAA |
| | | | AGAAAAAGTAAGAATTCCTAGAGCTCGCTGATC |
| | | | AGCCTCGACTGTGCCTTCTAGTTGCCAGCCATC |
| | | | TGTTGTTTGCCCCTCCCCCGTGCCTTCCTTGAC |
| | | | CCTGGAAGGTGCCACTCCCACTGTCCTTTCCTA |
| | | | ATAAAATGAGGAAATTGCATCGCATTGTCTGAG |
| | | | TAGGTGTCATTCTATTCTGGGGGGTGGGGTGGG |
| | | | GCAGGACAGCAAGGGGGAGGATTGGGAAGAGAA |
| | | | TAGCAGGCATGCTGGGGAGCGGCCGCAGGAACC |
| | | | CCTAGTGATGGAGTTGGCCACTCCCTCTCTGCG |
| | | | CGCTCGCTCGCTCACTGAGGCCGGGCGACCAAA |
| | | | GGTCGCCCGACGCCCGGGCTTTGCCCGGGCGGC |
| | | | CTCAGTGAGCGAGCGAGCGCGCAGCTGCCTGCA |
| | | | GGGGCGCCTGATGCGGTATTTTCTCCTTACGCA |
| | | | TCTGTGCGGTATTTCACACCGCATACGTCAAAG |
| | | | CAACCATAGTACGCGCCCTGTAGCGGCGCATTA |
| | | | AGCGCGGCGGGTGTGGTGGTTACGCGCAGCGTG |
| | | | ACCGCTACACTTGCCAGCGCCCTAGCGCCCGCT |
| | | | CCTTTCGCTTTCTTCCCTTCCTTTCTCGCCACG |
| | | | TTCGCCGGCTTTCCCCGTCAAGCTCTAAATCGG |
| | | | GGGCTCCCTTTAGGGTTCCGATTTAGTGCTTTA |
| | | | CGGCACCTCGACCCCAAAAAACTTGATTTGGGT |
| | | | GATGGTTCACGTAGTGGGCCATCGCCCTGATAG |
| | | | ACGGTTTTTCGCCCTTTGACGTTGGAGTCCACG |
| | | | TTCTTTAATAGTGGACTCTTGTTCCAAACTGGA |
| | | | ACAACACTCAACCCTATCTCGGGCTATTCTTTT |
| | | | GATTTATAAGGGATTTTGCCGATTTCGGCCTAT |
| | | | TGGTTAAAAAATGAGCTGATTTAACAAAAATTT |
| | | | AACGCGAATTTTAACAAAATATTAACGTTTACA |
| | | | ATTTTATGGTGCACTCTCAGTACAATCTGCTCT |
| | | | GATGCCGCATAGTTAAGCCAGCCCCGACACCCG |
| | | | CCAACACCCGCTGACGCGCCCTGACGGGCTTGT |
| | | | CTGCTCCCGGCATCCGCTTACAGACAAGCTGTG |
| | | | ACCGTCTCCGGGAGCTGCATGTGTCAGAGGTTT |
| | | | TCACCGTCATCACCGAAACGCGCGAGACGAAAG |
| | | | GGCCTCGTGATACGCCTATTTTTATAGGTTAAT |
| | | | GTCATGATAATAATGGTTTCTTAGACGTCAGGT |
| | | | GGCACTTTTCGGGGAAATGTGCGCGGAACCCCT |
| | | | ATTTGTTTATTTTTCTAAATACATTCAAATATG |
| | | | TATCCGCTCATGAGACAATAACCCTGATAAATG |
| | | | CTTCAATAATATTGAAAAAGGAAGAGTATGAGT |
| | | | ATTCAACATTTCCGTGTCGCCCTTATTCCCTTT |
| | | | TTTGCGGCATTTTGCCTTCCTGTTTTTGCTCAC |
| | | | CCAGAAACGCTGGTGAAAGTAAAAGATGCTGAA |
| | | | GATCAGTTGGGTGCACGAGTGGGTTACATCGAA |
| | | | CTGGATCTCAACAGCGGTAAGATCCTTGAGAGT |
| | | | TTTCGCCCCGAAGAACGTTTTCCAATGATGAGC |
| | | | ACTTTTAAAGTTCTGCTATGTGGCGCGGTATTA |
| | | | TCCCGTATTGACGCCGGGCAAGAGCAACTCGGT |
| | | | CGCCGCATACACTATTCTCAGAATGACTTGGTT |
| | | | GAGTACTCACCAGTCACAGAAAAGCATCTTACG |
| | | | GATGGCATGACAGTAAGAGAATTATGCAGTGCT |
| | | | GCCATAACCATGAGTGATAACACTGCGGCCAAC |
| | | | TTACTTCTGACAACGATCGGAGGACCGAAGGAG |
| | | | CTAACCGCTTTTTTGCACAACATGGGGGATCAT |
| | | | GTAACTCGCCTTGATCGTTGGGAACCGGAGCTG |
| | | | AATGAAGCCATACCAAACGACGAGCGTGACACC |
| | | | ACGATGCCTGTAGCAATGGCAACAACGTTGCGC |
| | | | AAACTATTAACTGGCGAACTACTTACTCTAGCT |
| | | | TCCCGGCAACAATTAATAGACTGGATGGAGGCG |
| | | | GATAAAGTTGCAGGACCACTTCTGCGCTCGGCC |
| | | | CTTCCGGCTGGCTGGTTTATTGCTGATAAATCT |
| | | | GGAGCCGGTGAGCGTGGAAGCCGCGGTATCATT |
| | | | GCAGCACTGGGGCCAGATGGTAAGCCCTCCCGT |
| | | | ATCGTAGTTATCTACACGACGGGGAGTCAGGCA |
| | | | ACTATGGATGAACGAAATAGACAGATCGCTGAG |
| | | | ATAGGTGCCTCACTGATTAAGCATTGGTAACTG |
| | | | TCAGACCAAGTTTACTCATATATACTTTAGATT |
| | | | GATTTAAAACTTCATTTTTAATTTAAAAGGATC |
| | | | TAGGTGAAGATCCTTTTTGATAATCTCATGACC |
| | | | AAAATCCCTTAACGTGAGTTTTCGTTCCACTGA |
| | | | GCGTCAGACCCCGTAGAAAAGATCAAAGGATCT |
| | | | TCTTGAGATCCTTTTTTTCTGCGCGTAATCTGC |
| | | | TGCTTGCAAACAAAAAAACCACCGCTACCAGCG |
| | | | GTGGTTTGTTTGCCGGATCAAGAGCTACCAACT |
| | | | CTTTTTCCGAAGGTAACTGGCTTCAGCAGAGCG |
| | | | CAGATACCAAATACTGTCCTTCTAGTGTAGCCG |
| | | | TAGTTAGGCCACCACTTCAAGAACTCTGTAGCA |
| | | | CCGCCTACATACCTCGCTCTGCTAATCCTGTTA |
| | | | CCAGTGGCTGCTGCCAGTGGCGATAAGTCGTGT |
| | | | CTTACCGGGTTGGACTCAAGACGATAGTTACCG |
| | | | GATAAGGCGCAGCGGTCGGGCTGAACGGGGGGT |
| | | | TCGTGCACACAGCCCAGCTTGGAGCGAACGACC |
| | | | TACACCGAACTGAGATACCTACAGCGTGAGCTA |
| | | | TGAGAAAGCGCCACGCTTCCCGAAGGGAGAAAG |
| | | | GCGGACAGGTATCCGGTAAGCGGCAGGGTCGGA |
| | | | ACAGGAGAGCGCACGAGGGAGCTTCCAGGGGGA |
| | | | AACGCCTGGTATCTTTATAGTCCTGTCGGGTTT |
| | | | CGCCACCTCTGACTTGAGCGTCGATTTTTGTGA |
| | | | TGCTCGTCAGGGGGGCGGAGCCTATGGAAAAAC |
| | | | GCCAGCAACGCGGCCTTTTTACGGTTCCTGGCC |
| | | | TTTTGCTGGCCTTTTGCTCACATGT |
| SEQ ID NO. | DNA | DNA coding | AATTCTGGTGATCCTCGGAGACACAGTCTGGGC |
| 207 | | sequence for R unit | GGTTCTCGTAAACCCGATCTGATTGCCTATAAA |
| | | for programmed | AACTTTGATCTGCTGGTCATTGTTCTTAAGCCT |
| | | gene regulation | |
| SEQ ID NO. | Amino | Amino acid | NSGDPRRHSLGGSRKPDLIAYKNFDLLVIVLKP |
| 208 | acid | sequence for R unit | |
| | | for programmed | |
| | | gene regulation | |
| SEQ ID NO. | DNA | DNA coding | AATTCTGGTGATCCTCGGAGACACAGTCTGGGC |
| 209 | | sequence for R unit | GGTTCTCGTAAACCCGATGGTGCTATTTATACT |
| | | for programmed | GTTGGTTCTCCTATTGATTATGGTGTTATTGTT |
| | | gene regulation | GTTACTAAACCT- |
| SEQ ID NO. | Amino | Amino acid | |
| 210 | acid | sequence for R unit | NSGDPRRHSLGGSRKPDGAIYTVGSPIDYGVIV |
| | | for programmed | VTKP |
| | | gene regulation | |
| SEQ ID NO. | DNA | DNA coding | AACTCTGGTGATCCTCGGAGACACAGTCTGGGC |
| 211 | | sequence for R unit | GGTTCTCGTAAACCCGATATTATTCTTGTTAAT |
| | | for programmed | GATAATATTTCTCTTATTCTTATTCTTGTTGCT |
| | | gene regulation | AAACCT |
| SEQ ID NO. | Amino | Amino acid | NSGDPRRHSLGGSRKPDIILVNDNISLILILVA |
| 212 | acid | sequence for R unit | KP |
| | | for programmed | |
| | | gene regulation | |
| SEQ ID NO. | DNA | DNA coding | AATTCTGGTGATCCTCGGAGACACAGTCTGGGC |
| 213 | | sequence of double | GGTTCTCGTAAACCCGATCTGATTGCCTATAAA |
| | | R units for | AACTTTGATCTGCTGGTCATTGTTCTTAAGCCT |
| | | programmed gene | AAATACTCCCAGAATTCTGGTGATCCTCGGAGA |
| | | regulation | CACAGTCTGGGCGGTTCTCGTAAACCCGATGGT |
| | | | GCTATTTATACTGTTGGTTCTCCTATTGATTAT |
| | | | GGTGTTATTGTTGTTACTAAACCT |
| SEQ ID NO. | Amino | Amino acid | NSGDPRRHSLGGSRKPDLIAYKNFDLLVIVLKP |
| 214 | acid | sequence of double | KYSQNSGDPRRHSLGGSRKPDGAIYTVGSPIDY |
| | | R units for | GVIVVTKP |
| | | programmed gene | |
| | | regulation | |
| SEQ ID NO. | DNA | DNA coding | AATTCTGGTGATCCTCGGAGACACAGTCTGGGC |
| 215 | | sequence for triple | GGTTCTCGTAAACCCGATCTGATTGCCTATAAA |
| | | R units for | AACTTTGATCTGCTGGTCATTGTTCTTAAGCCT |
| | | programmed gene | AAATACTCCCAGAATTCTGGTGATCCTCGGAGA |
| | | regulation | CACAGTCTGGGCGGTTCTCGTAAACCCGATGGT |
| | | | GCTATTTATACTGTTGGTTCTCCTATTGATTAT |
| | | | GGTGTTATTGTTGTTACTAAACCTAAGTACTCC |
| | | | CAGAACTCTGGTGATCCTCGGAGACACAGTCTG |
| | | | GGCGGTTCTCGTAAACCCGATATTATTCTTGTT |
| | | | AATGATAATATTTCTCTTATTCTTATTCTTGTT |
| | | | GCTAAACCT |
| SEQ ID NO. | Amino | Amino acid | NSGDPRRHSLGGSRKPDLIAYKNFDLLVIVLKP |
| 216 | acid | sequence for triple | KYSQNSGDPRRHSLGGSRKPDGAIYTVGSPIDY |
| | | R units for | GVIVVTKPKYSQNSGDPRRHSLGGSRKPDIILV |
| | | programmed gene | NDNISLILILVAKP |
| | | regulation | |
| SEQ ID NO. | DNA | pb74 full length | GACTCTTCGCGATGTACGGGCCAGATATACGCG |
| 217 | | sequence | TTGACATTGATTATTGACTAGTTATTAATAGTA |
| | | | ATCAATTACGGGGTCATTAGTTCATAGCCCATA |
| | | | TATGGAGTTCCGCGTTACATAACTTACGGTAAA |
| | | | TGGCCCGCCTGGCTGACCGCCCAACGACCCCCG |
| | | | CCCATTGACGTCAATAATGACGTATGTTCCCAT |
| | | | AGTAACGCCAATAGGGACTTTCCATTGACGTCA |
| | | | ATGGGTGGACTATTTACGGTAAACTGCCCACTT |
| | | | GGCAGTACATCAAGTGTATCATATGCCAAGTAC |
| | | | GCCCCCTATTGACGTCAATGACGGTAAATGGCC |
| | | | CGCCTGGCATTATGCCCAGTACATGACCTTATG |
| | | | GGACTTTCCTACTTGGCAGTACATCTACGTATT |
| | | | AGTCATCGCTATTACCATGGTGATGCGGTTTTG |
| | | | GCAGTACATCAATGGGCGTGGATAGCGGTTTGA |
| | | | CTCACGGGGATTTCCAAGTCTCCACCCCATTGA |
| | | | CGTCAATGGGAGTTTGTTTTGGCACCAAAATCA |
| | | | ACGGGACTTTCCAAAATGTCGTAACAACTCCGC |
| | | | CCCATTGACGCAAATGGGCGGTAGGCGTGTACG |
| | | | GTGGGAGGTCTATATAAGCAGAGCTCTCTGGCT |
| | | | AACTAGAGAACCCACTGCTTACTGGCTTATCGA |
| | | | AATTAATACGACTCACTATAGGGAGACCCAAGC |
| | | | TGGCTAGCGTTTAAACTTAAGCTTATGGCGGCG |
| | | | ATGGCCGAGCGGCCCTTCCAGTGCAGGATCTGT |
| | | | ATGCGCAACTTTTCTCAGTCCGGCGACCTGACC |
| | | | CGGCACATCAGAACCCATACAGGCGAAAAGCCT |
| | | | TTCGCCTGCGACATTTGTGGGAGAAAATTTGCT |
| | | | CGGTCCGACAACCTGACCACCCATACCAAGATC |
| | | | CACACCGGCTCTCAGAAACCATTCCAGTGCCGC |
| | | | ATTTGTATGCGGAATTTTTCCCGGTCCTCCGAC |
| | | | CTGACCCGGCATATCCGCACTCACACCGGAGAG |
| | | | AAGCCCTTTGCTTGCGACATTTGTGGCAGGAAA |
| | | | TTTGCTCGGTCCGACGCCCTGACCCGGCACACT |
| | | | AAGATCCATACTGGGTCACAGAAACCTTTCCAG |
| | | | TGCCGGATTTGTATGAGAAACTTTAGCCGGTCC |
| | | | GACGCCCTGTCCGAGCACATCAGAACCCATACA |
| | | | GGCGAAAAGCCTTTCGCCTGCGACATTTGTGGG |
| | | | AGAAAATTTGCTCGGTCCTCCAACCTGACCCGG |
| | | | CATACCAAGATCCACACCGGCTCTCAGAAACCA |
| | | | TTCCAGTGCCGCATTTGTATGCGGAATTTTTCC |
| | | | CGGTCCGACGCCCTGACCACCCACATCAGAACA |
| | | | CATACTGGGCTGAGAGGATCCAATTCTGGTGAT |
| | | | CCTCGGAGACACAGTCTGGGCGGTTCTCGTAAA |
| | | | CCCGATCTGATTGCCTATAAAAACTTTGATCTG |
| | | | CTGGTCATTGTTCTTAAGCCTTGAGCGGCCGCT |
| | | | CGAGTCTAGAGGGCCCGTTTAAACCCGCTGATC |
| | | | AGCCTCGACTGTGCCTTCTAGTTGCCAGCCATC |
| | | | TGTTGTTTGCCCCTCCCCCGTGCCTTCCTTGAC |
| | | | CCTGGAAGGTGCCACTCCCACTGTCCTTTCCTA |
| | | | ATAAAATGAGGAAATTGCATCGCATTGTCTGAG |
| | | | TAGGTGTCATTCTATTCTGGGGGGTGGGGTGGG |
| | | | GCAGGACAGCAAGGGGGAGGATTGGGAAGACAA |
| | | | TAGCAGGCATGCTGGGGATGCGGTGGGCTCTAT |
| | | | GGCTTCTACTGGGCGGTTTTATGGACAGCAAGC |
| | | | GAACCGGAATTGCCAGCTGGGGCGCCCTCTGGT |
| | | | AAGGTTGGGAAGCCCTGCAAAGTAAACTGGATG |
| | | | GCTTTCTCGCCGCCAAGGATCTGATGGCGCAGG |
| | | | GGATCAAGCTCTGATCAAGAGACAGGATGAGGA |
| | | | TCGTTTCGCATGATTGAACAAGATGGATTGCAC |
| | | | GCAGGTTCTCCGGCCGCTTGGGTGGAGAGGCTA |
| | | | TTCGGCTATGACTGGGCACAACAGACAATCGGC |
| | | | TGCTCTGATGCCGCCGTGTTCCGGCTGTCAGCG |
| | | | CAGGGGCGCCCGGTTCTTTTTGTCAAGACCGAC |
| | | | CTGTCCGGTGCCCTGAATGAACTGCAAGACGAG |
| | | | GCAGCGCGGCTATCGTGGCTGGCCACGACGGGC |
| | | | GTTCCTTGCGCAGCTGTGCTCGACGTTGTCACT |
| | | | GAAGCGGGAAGGGACTGGCTGCTATTGGGCGAA |
| | | | GTGCCGGGGCAGGATCTCCTGTCATCTCACCTT |
| | | | GCTCCTGCCGAGAAAGTATCCATCATGGCTGAT |
| | | | GCAATGCGGCGGCTGCATACGCTTGATCCGGCT |
| | | | ACCTGCCCATTCGACCACCAAGCGAAACATCGC |
| | | | ATCGAGCGAGCACGTACTCGGATGGAAGCCGGT |
| | | | CTTGTCGATCAGGATGATCTGGACGAAGAGCAT |
| | | | CAGGGGCTCGCGCCAGCCGAACTGTTCGCCAGG |
| | | | CTCAAGGCGAGCATGCCCGACGGCGAGGATCTC |
| | | | GTCGTGACCCATGGCGATGCCTGCTTGCCGAAT |
| | | | ATCATGGTGGAAAATGGCCGCTTTTCTGGATTC |
| | | | ATCGACTGTGGCCGGCTGGGTGTGGCGGACCGC |
| | | | TATCAGGACATAGCGTTGGCTACCCGTGATATT |
| | | | GCTGAAGAGCTTGGCGGCGAATGGGCTGACCGC |
| | | | TTCCTCGTGCTTTACGGTATCGCCGCTCCCGAT |
| | | | TCGCAGCGCATCGCCTTCTATCGCCTTCTTGAC |
| | | | GAGTTCTTCTGAATTATTAACGCTTACAATTTC |
| | | | CTGATGCGGTATTTTCTCCTTACGCATCTGTGC |
| | | | GGTATTTCACACCGCATACAGGTGGCACTTTTC |
| | | | GGGGAAATGTGCGCGGAACCCCTATTTGTTTAT |
| | | | TTTTCTAAATACATTCAAATATGTATCCGCTCA |
| | | | TGAGACAATAACCCTGATAAATGCTTCAATAAT |
| | | | AGCACGTGCTAAAACTTCATTTTTAATTTAAAA |
| | | | GGATCTAGGTGAAGATCCTTTTTGATAATCTCA |
| | | | TGACCAAAATCCCTTAACGTGAGTTTTCGTTCC |
| | | | ACTGAGCGTCAGACCCCGTAGAAAAGATCAAAG |
| | | | GATCTTCTTGAGATCCTTTTTTTCTGCGCGTAA |
| | | | TCTGCTGCTTGCAAACAAAAAAACCACCGCTAC |
| | | | CAGCGGTGGTTTGTTTGCCGGATCAAGAGCTAC |
| | | | CAACTCTTTTTCCGAAGGTAACTGGCTTCAGCA |
| | | | GAGCGCAGATACCAAATACTGTCCTTCTAGTGT |
| | | | AGCCGTAGTTAGGCCACCACTTCAAGAACTCTG |
| | | | TAGCACCGCCTACATACCTCGCTCTGCTAATCC |
| | | | TGTTACCAGTGGCTGCTGCCAGTGGCGATAAGT |
| | | | CGTGTCTTACCGGGTTGGACTCAAGACGATAGT |
| | | | TACCGGATAAGGCGCAGCGGTCGGGCTGAACGG |
| | | | GGGGTTCGTGCACACAGCCCAGCTTGGAGCGAA |
| | | | CGACCTACACCGAACTGAGATACCTACAGCGTG |
| | | | AGCTATGAGAAAGCGCCACGCTTCCCGAAGGGA |
| | | | GAAAGGCGGACAGGTATCCGGTAAGCGGCAGGG |
| | | | TCGGAACAGGAGAGCGCACGAGGGAGCTTCCAG |
| | | | GGGGAAACGCCTGGTATCTTTATAGTCCTGTCG |
| | | | GGTTTCGCCACCTCTGACTTGAGCGTCGATTTT |
| | | | TGTGATGCTCGTCAGGGGGGCGGAGCCTATGGA |
| | | | AAAACGCCAGCAACGCGGCCTTTTTACGGTTCC |
| | | | TGGGCTTTTGCTGGCCTTTTGCTCACATGTTCT |
| | | | T |
| SEQ ID NO. | DNA | pb74 cDNA | ATGGCGGCGATGGCCGAGCGGCCCTTCCAGTGC |
| 218 | | sequence | AGGATCTGTATGCGCAACTTTTCTCAGTCCGGC |
| | | | GACCTGACCCGGCACATCAGAACCCATACAGGC |
| | | | GAAAAGCCTTTCGCCTGCGACATTTGTGGGAGA |
| | | | AAATTTGCTCGGTCCGACAACCTGACCACCCAT |
| | | | ACCAAGATCCACACCGGCTCTCAGAAACCATTC |
| | | | CAGTGCCGCATTTGTATGCGGAATTTTTCCCGG |
| | | | TCCTCCGACCTGACCCGGCATATCCGCACTCAC |
| | | | ACCGGAGAGAAGCCCTTTGCTTGCGACATTTGT |
| | | | GGCAGGAAATTTGCTCGGTCCGACGCCCTGACC |
| | | | CGGCACACTAAGATCCATACTGGGTCACAGAAA |
| | | | CCTTTCCAGTGCCGGATTTGTATGAGAAACTTT |
| | | | AGCCGGTCCGACGCCCTGTCCGAGCACATCAGA |
| | | | ACCCATACAGGCGAAAAGCCTTTCGCCTGCGAC |
| | | | ATTTGTGGGAGAAAATTTGCTCGGTCCTCCAAC |
| | | | CTGACCCGGCATACCAAGATCCACACCGGCTCT |
| | | | CAGAAACCATTCCAGTGCCGCATTTGTATGCGG |
| | | | AATTTTTCCCGGTCCGACGCCCTGACCACCCAC |
| | | | ATCAGAACACATACTGGGCTGAGAGGATCCAAT |
| | | | TCTGGTGATCCTCGGAGACACAGTCTGGGCGGT |
| | | | TCTCGTAAACCCGATCTGATTGCCTATAAAAAC |
| | | | TTTGATCTGCTGGTCATTGTTCTTAAGCCTTGA |
| SEQ ID NO. | Amino | Amino acid | MAAMAERPFQCRICMRNFSQSGDLTRHIRTHTG |
| 219 | acid | sequence encoded in | EKPFACDICGRKFARSDNLTTHTKIHTGSQKPF |
| | | pb74 | QCRICMRNFSRSSDLTRHIRTHTGEKPFACDIC |
| | | | GRKFARSDALTRHTKIHTGSQKPFQCRICMRNF |
| | | | SRSDALSEHIRTHTGEKPFACDICGRKFARSSN |
| | | | LTRHTKIHTGSQKPFQCRICMRNFSRSDALTTH |
| | | | IRTHTGLRGSNSGDPRRHSLGGSRKPDLIAYKN |
| | | | FDLLVIVLKP* |
| SEQ ID NO. | DNA | pb75 full length | GACTCTTCGCGATGTACGGGCCAGATATACGCG |
| 220 | | sequence | TTGACATTGATTATTGACTAGTTATTAATAGTA |
| | | | ATCAATTACGGGGTCATTAGTTCATAGCCCATA |
| | | | TATGGAGTTCCGCGTTACATAACTTACGGTAAA |
| | | | TGGCCCGCCTGGCTGACCGCCCAACGACCCCCG |
| | | | CCCATTGACGTCAATAATGACGTATGTTCCCAT |
| | | | AGTAACGCCAATAGGGACTTTCCATTGACGTCA |
| | | | ATGGGTGGACTATTTACGGTAAACTGCCCACTT |
| | | | GGCAGTACATCAAGTGTATCATATGCCAAGTAC |
| | | | GCCCCCTATTGACGTCAATGACGGTAAATGGCC |
| | | | CGCCTGGCATTATGCCCAGTACATGACCTTATG |
| | | | GGACTTTCCTACTTGGCAGTACATCTACGTATT |
| | | | AGTCATCGCTATTACCATGGTGATGCGGTTTTG |
| | | | GCAGTACATCAATGGGCGTGGATAGCGGTTTGA |
| | | | CTCACGGGGATTTCCAAGTCTCCACCCCATTGA |
| | | | CGTCAATGGGAGTTTGTTTTGGCACCAAAATCA |
| | | | ACGGGACTTTCCAAAATGTCGTAACAACTCCGC |
| | | | CCCATTGACGCAAATGGGCGGTAGGCGTGTACG |
| | | | GTGGGAGGTCTATATAAGCAGAGCTCTCTGGCT |
| | | | AACTAGAGAACCCACTGCTTACTGGCTTATCGA |
| | | | AATTAATACGACTCACTATAGGGAGACCCAAGC |
| | | | TGGCTAGCGTTTAAACTTAAGCTTATGGCGGCG |
| | | | ATGGCCGAGCGGCCCTTCCAGTGCAGGATCTGT |
| | | | ATGCGCAACTTTTCTCAGTCCGGCGACCTGACC |
| | | | CGGCACATCAGAACCCATACAGGCGAAAAGCCT |
| | | | TTCGCCTGCGACATTTGTGGGAGAAAATTTGCT |
| | | | CGGTCCGACAACCTGACCACCCATACCAAGATC |
| | | | CACACCGGCTCTCAGAAACCATTCCAGTGCCGC |
| | | | ATTTGTATGCGGAATTTTTCCCGGTCCTCCGAC |
| | | | CTGACCCGGCATATCCGCACTCACACCGGAGAG |
| | | | AAGCCCTTTGCTTGCGACATTTGTGGCAGGAAA |
| | | | TTTGCTCGGTCCGACGCCCTGACCCGGCACACT |
| | | | AAGATCCATACTGGGTCACAGAAACCTTTCCAG |
| | | | TGCCGGATTTGTATGAGAAACTTTAGCCGGTCC |
| | | | GACGCCCTGTCCGAGCACATCAGAACCCATACA |
| | | | GGCGAAAAGCCTTTCGCCTGCGACATTTGTGGG |
| | | | AGAAAATTTGCTCGGTCCTCCAACCTGACCCGG |
| | | | CATACCAAGATCCACACCGGCTCTCAGAAACCA |
| | | | TTCCAGTGCCGCATTTGTATGCGGAATTTTTCC |
| | | | CGGTCCGACGCCCTGACCACCCACATCAGAACA |
| | | | CATACTGGGCTGAGAGGATCCAATTCTGGTGAT |
| | | | CCTCGGAGACACAGTCTGGGCGGTTCTCGTAAA |
| | | | CCCGATCTGATTGCCTATAAAAACTTTGATCTG |
| | | | CTGGTCATTGTTCTTAAGCCTAAATACTCCCAG |
| | | | AATTCTGGTGATCCTCGGAGACACAGTCTGGGC |
| | | | GGTTCTCGTAAACCCGATGGTGCTATTTATACT |
| | | | GTTGGTTCTCCTATTGATTATGGTGTTATTGTT |
| | | | GTTACTAAACCTTGAGCGGCCGCTCGAGTCTAG |
| | | | AGGGCCCGTTTAAACCCGCTGATCAGCCTCGAC |
| | | | TGTGCCTTCTAGTTGCCAGCCATCTGTTGTTTG |
| | | | CCCCTCCCCCGTGCCTTCCTTGACCCTGGAAGG |
| | | | TGCCACTCCCACTGTCCTTTCCTAATAAAATGA |
| | | | GGAAATTGCATCGCATTGTCTGAGTAGGTGTCA |
| | | | TTCTATTCTGGGGGGTGGGGTGGGGCAGGACAG |
| | | | CAAGGGGGAGGATTGGGAAGACAATAGCAGGCA |
| | | | TGCTGGGGATGCGGTGGGCTCTATGGCTTCTAC |
| | | | TGGGCGGTTTTATGGACAGCAAGCGAACCGGAA |
| | | | TTGCCAGCTGGGGCGCCCTCTGGTAAGGTTGGG |
| | | | AAGCCCTGCAAAGTAAACTGGATGGCTTTCTCG |
| | | | CCGCCAAGGATCTGATGGCGCAGGGGATCAAGC |
| | | | TCTGATCAAGAGACAGGATGAGGATCGTTTCGC |
| | | | ATGATTGAACAAGATGGATTGCACGCAGGTTCT |
| | | | CCGGCCGCTTGGGTGGAGAGGCTATTCGGCTAT |
| | | | GACTGGGCACAACAGACAATCGGCTGCTCTGAT |
| | | | GCCGCCGTGTTCCGGCTGTCAGCGCAGGGGCGC |
| | | | CCGGTTCTTTTTGTCAAGACCGACCTGTCCGGT |
| | | | GCCCTGAATGAACTGCAAGACGAGGCAGCGCGG |
| | | | CTATCGTGGCTGGCCACGACGGGCGTTCCTTGC |
| | | | GCAGCTGTGCTCGACGTTGTCACTGAAGCGGGA |
| | | | AGGGACTGGCTGCTATTGGGCGAAGTGCCGGGG |
| | | | CAGGATCTCCTGTCATCTCACCTTGCTCCTGCC |
| | | | GAGAAAGTATCCATCATGGCTGATGCAATGCGG |
| | | | CGGCTGCATACGCTTGATCCGGCTACCTGCCCA |
| | | | TTCGACCACCAAGCGAAACATCGCATCGAGCGA |
| | | | GCACGTACTCGGATGGAAGCCGGTCTTGTCGAT |
| | | | CAGGATGATCTGGACGAAGAGCATCAGGGGCTC |
| | | | GCGCCAGCCGAACTGTTCGCCAGGCTCAAGGCG |
| | | | AGCATGCCCGACGGCGAGGATCTCGTCGTGACC |
| | | | CATGGCGATGCCTGCTTGCCGAATATCATGGTG |
| | | | GAAAATGGCCGCTTTTCTGGATTCATCGACTGT |
| | | | GGCCGGCTGGGTGTGGCGGACCGCTATCAGGAC |
| | | | ATAGCGTTGGCTACCCGTGATATTGCTGAAGAG |
| | | | CTTGGCGGCGAATGGGCTGACCGCTTCCTCGTG |
| | | | CTTTACGGTATCGCCGCTCCCGATTCGCAGCGC |
| | | | ATCGCCTTCTATCGCCTTCTTGACGAGTTCTTC |
| | | | TGAATTATTAACGCTTACAATTTCCTGATGCGG |
| | | | TATTTTCTCCTTACGCATCTGTGCGGTATTICA |
| | | | CACCGCATACAGGTGGCACTTTTCGGGGAAATG |
| | | | TGCGCGGAACCCCTATTTGTTTATTTTTCTAAA |
| | | | TACATTCAAATATGTATCCGCTCATGAGACAAT |
| | | | AACCCTGATAAATGCTTCAATAATAGCACGTGC |
| | | | TAAAACTTCATTTTTAATTTAAAAGGATCTAGG |
| | | | TGAAGATCCTTTTTGATAATCTCATGACCAAAA |
| | | | TCCCTTAACGTGAGTTTTCGTTCCACTGAGCGT |
| | | | CAGACCCCGTAGAAAAGATCAAAGGATCTTCTT |
| | | | GAGATCCTTTTTTTCTGCGCGTAATCTGCTGCT |
| | | | TGCAAACAAAAAAACCACCGCTACCAGCGGTGG |
| | | | TTTGTTTGCCGGATCAAGAGCTACCAACTCTTT |
| | | | TTCCGAAGGTAACTGGCTTCAGCAGAGCGCAGA |
| | | | TACCAAATACTGTCCTTCTAGTGTAGCCGTAGT |
| | | | TAGGCCACCACTTCAAGAACTCTGTAGCACCGC |
| | | | CTACATACCTCGCTCTGCTAATCCTGTTACCAG |
| | | | TGGCTGCTGCCAGTGGCGATAAGTCGTGTCTTA |
| | | | CCGGGTTGGACTCAAGACGATAGTTACCGGATA |
| | | | AGGCGCAGCGGTCGGGCTGAACGGGGGGTTCGT |
| | | | GCACACAGCCCAGCTTGGAGCGAACGACCTACA |
| | | | CCGAACTGAGATACCTACAGCGTGAGCTATGAG |
| | | | AAAGCGCCACGCTTCCCGAAGGGAGAAAGGCGG |
| | | | ACAGGTATCCGGTAAGCGGCAGGGTCGGAACAG |
| | | | GAGAGCGCACGAGGGAGCTTCCAGGGGGAAACG |
| | | | CCTGGTATCTTTATAGTCCTGTCGGGTTTCGCC |
| | | | ACCTCTGACTTGAGCGTCGATTTTTGTGATGCT |
| | | | CGTCAGGGGGGCGGAGCCTATGGAAAAACGCCA |
| | | | GCAACGCGGCCTTTTTACGGTTCCTGGGCTTTT |
| | | | GCTGGCCTTTTGCTCACATGTTCTT |
| SEQ ID NO. | DNA | pb75 cDNA | ATGGCGGCGATGGCCGAGCGGCCCTTCCAGTGC |
| 221 | | sequence | AGGATCTGTATGCGCAACTTTTCTCAGTCCGGC |
| | | | GACCTGACCCGGCACATCAGAACCCATACAGGC |
| | | | GAAAAGCCTTTCGCCTGCGACATTTGTGGGAGA |
| | | | AAATTTGCTCGGTCCGACAACCTGACCACCCAT |
| | | | ACCAAGATCCACACCGGCTCTCAGAAACCATTC |
| | | | CAGTGCCGCATTTGTATGCGGAATTTTTCCCGG |
| | | | TCCTCCGACCTGACCCGGCATATCCGCACTCAC |
| | | | ACCGGAGAGAAGCCCTTTGCTTGCGACATTTGT |
| | | | GGCAGGAAATTTGCTCGGTCCGACGCCCTGACC |
| | | | CGGCACACTAAGATCCATACTGGGTCACAGAAA |
| | | | CCTTTCCAGTGCCGGATTTGTATGAGAAACTTT |
| | | | AGCCGGTCCGACGCCCTGTCCGAGCACATCAGA |
| | | | ACCCATACAGGCGAAAAGCCTTTCGCCTGCGAC |
| | | | ATTTGTGGGAGAAAATTTGCTCGGTCCTCCAAC |
| | | | CTGACCCGGCATACCAAGATCCACACCGGCTCT |
| | | | CAGAAACCATTCCAGTGCCGCATTTGTATGCGG |
| | | | AATTTTTCCCGGTCCGACGCCCTGACCACCCAC |
| | | | ATCAGAACACATACTGGGCTGAGAGGATCCAAT |
| | | | TCTGGTGATCCTCGGAGACACAGTCTGGGCGGT |
| | | | TCTCGTAAACCCGATCTGATTGCCTATAAAAAC |
| | | | TTTGATCTGCTGGTCATTGTTCTTAAGCCTAAA |
| | | | TACTCCCAGAATTCTGGTGATCCTCGGAGACAC |
| | | | AGTCTGGGCGGTTCTCGTAAACCCGATGGTGCT |
| | | | ATTTATACTGTTGGTTCTCCTATTGATTATGGT |
| | | | GTTATTGTTGTTACTAAACCTTGA |
| SEQ ID NO. | Amino | Amino acid | MAAMAERPFQCRICMRNFSQSGDLTRHIRTHTG |
| 222 | acid | sequence encoded in | EKPFACDICGRKFARSDNLTTHTKIHTGSQKPF |
| | | pb75 | QCRICMRNFSRSSDLTRHIRTHTGEKPFACDIC |
| | | | GRKFARSDALTRHTKIHTGSQKPFQCRICMRNF |
| | | | SRSDALSEHIRTHTGEKPFACDICGRKFARSSN |
| | | | LTRHTKIHTGSQKPFQCRICMRNESRSDALTTH |
| | | | IRTHTGLRGSNSGDPRRHSLGGSRKPDLIAYKN |
| | | | FDLLVIVLKPKYSQNSGDPRRHSLGGSRKPDGA |
| | | | IYTVGSPIDYGVIVVTKP* |
| SEQ ID NO. | DNA | pb76 full length | GACTCTTCGCGATGTACGGGCCAGATATACGCG |
| 223 | | sequence | TTGACATTGATTATTGACTAGTTATTAATAGTA |
| | | | ATCAATTACGGGGTCATTAGTTCATAGCCCATA |
| | | | TATGGAGTTCCGCGTTACATAACTTACGGTAAA |
| | | | TGGCCCGCCTGGCTGACCGCCCAACGACCCCCG |
| | | | CCCATTGACGTCAATAATGACGTATGTTCCCAT |
| | | | AGTAACGCCAATAGGGACTTTCCATTGACGTCA |
| | | | ATGGGTGGACTATTTACGGTAAACTGCCCACTT |
| | | | GGCAGTACATCAAGTGTATCATATGCCAAGTAC |
| | | | GCCCCCTATTGACGTCAATGACGGTAAATGGCC |
| | | | CGCCTGGCATTATGCCCAGTACATGACCTTATG |
| | | | GGACTTTCCTACTTGGCAGTACATCTACGTATT |
| | | | AGTCATCGCTATTACCATGGTGATGCGGTTTTG |
| | | | GCAGTACATCAATGGGCGTGGATAGCGGTTTGA |
| | | | CTCACGGGGATTTCCAAGTCTCCACCCCATTGA |
| | | | CGTCAATGGGAGTTTGTTTTGGCACCAAAATCA |
| | | | ACGGGACTTTCCAAAATGTCGTAACAACTCCGC |
| | | | CCCATTGACGCAAATGGGCGGTAGGCGTGTACG |
| | | | GTGGGAGGTCTATATAAGCAGAGCTCTCTGGCT |
| | | | AACTAGAGAACCCACTGCTTACTGGCTTATCGA |
| | | | AATTAATACGACTCACTATAGGGAGACCCAAGC |
| | | | TGGCTAGCGTTTAAACTTAAGCTTATGGCGGCG |
| | | | ATGGCCGAGCGGCCCTTCCAGTGCAGGATCTGT |
| | | | ATGCGCAACTTTTCTCAGTCCGGCGACCTGACC |
| | | | CGGCACATCAGAACCCATACAGGCGAAAAGCCT |
| | | | TTCGCCTGCGACATTTGTGGGAGAAAATTTGCT |
| | | | CGGTCCGACAACCTGACCACCCATACCAAGATC |
| | | | CACACCGGCTCTCAGAAACCATTCCAGTGCCGC |
| | | | ATTTGTATGCGGAATTTTTCCCGGTCCTCCGAC |
| | | | CTGACCCGGCATATCCGCACTCACACCGGAGAG |
| | | | AAGCCCTTTGCTTGCGACATTTGTGGCAGGAAA |
| | | | TTTGCTCGGTCCGACGCCCTGACCCGGCACACT |
| | | | AAGATCCATACTGGGTCACAGAAACCTTTCCAG |
| | | | TGCCGGATTTGTATGAGAAACTTTAGCCGGTCC |
| | | | GACGCCCTGTCCGAGCACATCAGAACCCATACA |
| | | | GGCGAAAAGCCTTTCGCCTGCGACATTTGTGGG |
| | | | AGAAAATTTGCTCGGTCCTCCAACCTGACCCGG |
| | | | CATACCAAGATCCACACCGGCTCTCAGAAACCA |
| | | | TTCCAGTGCCGCATTTGTATGCGGAATTTTTCC |
| | | | CGGTCCGACGCCCTGACCACCCACATCAGAACA |
| | | | CATACTGGGCTGAGAGGATCCAATTCTGGTGAT |
| | | | CCTCGGAGACACAGTCTGGGCGGTTCTCGTAAA |
| | | | CCCGATCTGATTGCCTATAAAAACTTTGATCTG |
| | | | CTGGTCATTGTTCTTAAGCCTAAATACTCCCAG |
| | | | AATTCTGGTGATCCTCGGAGACACAGTCTGGGC |
| | | | GGTTCTCGTAAACCCGATGGTGCTATTTATACT |
| | | | GTTGGTTCTCCTATTGATTATGGTGTTATTGTT |
| | | | GTTACTAAACCTAAGTACTCCCAGAACTCTGGT |
| | | | GATCCTCGGAGACACAGTCTGGGCGGTTCTCGT |
| | | | AAACCCGATATTATTCTTGTTAATGATAATATT |
| | | | TCTCTTATTCTTATTCTTGTTGCTAAACCTTGA |
| | | | GCGGCCGCTCGAGTCTAGAGGGCCCGTTTAAAC |
| | | | CCGCTGATCAGCCTCGACTGTGCCTTCTAGTTG |
| | | | CCAGCCATCTGTTGTTTGCCCCTCCCCCGTGCC |
| | | | TTCCTTGACCCTGGAAGGTGCCACTCCCACTGT |
| | | | CCTTTCCTAATAAAATGAGGAAATTGCATCGCA |
| | | | TTGTCTGAGTAGGTGTCATTCTATTCTGGGGGG |
| | | | TGGGGTGGGGCAGGACAGCAAGGGGGAGGATTG |
| | | | GGAAGACAATAGCAGGCATGCTGGGGATGCGGT |
| | | | GGGCTCTATGGCTTCTACTGGGCGGTTTTATGG |
| | | | ACAGCAAGCGAACCGGAATTGCCAGCTGGGGCG |
| | | | CCCTCTGGTAAGGTTGGGAAGCCCTGCAAAGTA |
| | | | AACTGGATGGCTTTCTCGCCGCCAAGGATCTGA |
| | | | TGGCGCAGGGGATCAAGCTCTGATCAAGAGACA |
| | | | GGATGAGGATCGTTTCGCATGATTGAACAAGAT |
| | | | GGATTGCACGCAGGTTCTCCGGCCGCTTGGGTG |
| | | | GAGAGGCTATTCGGCTATGACTGGGCACAACAG |
| | | | ACAATCGGCTGCTCTGATGCCGCCGTGTTCCGG |
| | | | CTGTCAGCGCAGGGGCGCCCGGTTCTTTTTGTC |
| | | | AAGACCGACCTGTCCGGTGCCCTGAATGAACTG |
| | | | CAAGACGAGGCAGCGCGGCTATCGTGGCTGGCC |
| | | | ACGACGGGCGTTCCTTGCGCAGCTGTGCTCGAC |
| | | | GTTGTCACTGAAGCGGGAAGGGACTGGCTGCTA |
| | | | TTGGGCGAAGTGCCGGGGCAGGATCTCCTGTCA |
| | | | TCTCACCTTGCTCCTGCCGAGAAAGTATCCATC |
| | | | ATGGCTGATGCAATGCGGCGGCTGCATACGCTT |
| | | | GATCCGGCTACCTGCCCATTCGACCACCAAGCG |
| | | | AAACATCGCATCGAGCGAGCACGTACTCGGATG |
| | | | GAAGCCGGTCTTGTCGATCAGGATGATCTGGAC |
| | | | GAAGAGCATCAGGGGCTCGCGCCAGCCGAACTG |
| | | | TTCGCCAGGCTCAAGGCGAGCATGCCCGACGGC |
| | | | GAGGATCTCGTCGTGACCCATGGCGATGCCTGC |
| | | | TTGCCGAATATCATGGTGGAAAATGGCCGCTTT |
| | | | TCTGGATTCATCGACTGTGGCCGGCTGGGTGTG |
| | | | GCGGACCGCTATCAGGACATAGCGTTGGCTACC |
| | | | CGTGATATTGCTGAAGAGCTTGGCGGCGAATGG |
| | | | GCTGACCGCTTCCTCGTGCTTTACGGTATCGCC |
| | | | GCTCCCGATTCGCAGCGCATCGCCTTCTATCGC |
| | | | CTTCTTGACGAGTTCTTCTGAATTATTAACGCT |
| | | | TACAATTTCCTGATGCGGTATTTTCTCCTTACG |
| | | | CATCTGTGCGGTATTTCACACCGCATACAGGTG |
| | | | GCACTTTTCGGGGAAATGTGCGCGGAACCCCTA |
| | | | TTTGTTTATTTTTCTAAATACATTCAAATATGT |
| | | | ATCCGCTCATGAGACAATAACCCTGATAAATGC |
| | | | TTCAATAATAGCACGTGCTAAAACTTCATTTTT |
| | | | AATTTAAAAGGATCTAGGTGAAGATCCTTTTTG |
| | | | ATAATCTCATGACCAAAATCCCTTAACGTGAGT |
| | | | TTTCGTTCCACTGAGCGTCAGACCCCGTAGAAA |
| | | | AGATCAAAGGATCTTCTTGAGATCCTTTTTTTC |
| | | | TGCGCGTAATCTGCTGCTTGCAAACAAAAAAAC |
| | | | CACCGCTACCAGCGGTGGTTTGTTTGCCGGATC |
| | | | AAGAGCTACCAACTCTTTTTCCGAAGGTAACTG |
| | | | GCTTCAGCAGAGCGCAGATACCAAATACTGTCC |
| | | | TTCTAGTGTAGCCGTAGTTAGGCCACCACTTCA |
| | | | AGAACTCTGTAGCACCGCCTACATACCTCGCTC |
| | | | TGCTAATCCTGTTACCAGTGGCTGCTGCCAGTG |
| | | | GCGATAAGTCGTGTCTTACCGGGTTGGACTCAA |
| | | | GACGATAGTTACCGGATAAGGCGCAGCGGTCGG |
| | | | GCTGAACGGGGGGTTCGTGCACACAGCCCAGCT |
| | | | TGGAGCGAACGACCTACACCGAACTGAGATACC |
| | | | TACAGCGTGAGCTATGAGAAAGCGCCACGCTTC |
| | | | CCGAAGGGAGAAAGGCGGACAGGTATCCGGTAA |
| | | | GCGGCAGGGTCGGAACAGGAGAGCGCACGAGGG |
| | | | AGCTTCCAGGGGGAAACGCCTGGTATCTTTATA |
| | | | GTCCTGTCGGGTTTCGCCACCTCTGACTTGAGC |
| | | | GTCGATTTTTGTGATGCTCGTCAGGGGGGCGGA |
| | | | GCCTATGGAAAAACGCCAGCAACGCGGCCTTTT |
| | | | TACGGTTCCTGGGCTTTTGCTGGCCTTTTGCTC |
| | | | ACATGTTCTT |
| SEQ ID NO. | DNA | pb76 cDNA | ATGGCGGCGATGGCCGAGCGGCCCTTCCAGTGC |
| 224 | | sequence | AGGATCTGTATGCGCAACTTTTCTCAGTCCGGC |
| | | | GACCTGACCCGGCACATCAGAACCCATACAGGC |
| | | | GAAAAGCCTTTCGCCTGCGACATTTGTGGGAGA |
| | | | AAATTTGCTCGGTCCGACAACCTGACCACCCAT |
| | | | ACCAAGATCCACACCGGCTCTCAGAAACCATTC |
| | | | CAGTGCCGCATTTGTATGCGGAATTTTTCCCGG |
| | | | TCCTCCGACCTGACCCGGCATATCCGCACTCAC |
| | | | ACCGGAGAGAAGCCCTTTGCTTGCGACATTTGT |
| | | | GGCAGGAAATTTGCTCGGTCCGACGCCCTGACC |
| | | | CGGCACACTAAGATCCATACTGGGTCACAGAAA |
| | | | CCTTTCCAGTGCCGGATTTGTATGAGAAACTTT |
| | | | AGCCGGTCCGACGCCCTGTCCGAGCACATCAGA |
| | | | ACCCATACAGGCGAAAAGCCTTTCGCCTGCGAC |
| | | | ATTTGTGGGAGAAAATTTGCTCGGTCCTCCAAC |
| | | | CTGACCCGGCATACCAAGATCCACACCGGCTCT |
| | | | CAGAAACCATTCCAGTGCCGCATTTGTATGCGG |
| | | | AATTTTTCCCGGTCCGACGCCCTGACCACCCAC |
| | | | ATCAGAACACATACTGGGCTGAGAGGATCCAAT |
| | | | TCTGGTGATCCTCGGAGACACAGTCTGGGCGGT |
| | | | TCTCGTAAACCCGATCTGATTGCCTATAAAAAC |
| | | | TTTGATCTGCTGGTCATTGTTCTTAAGCCTAAA |
| | | | TACTCCCAGAATTCTGGTGATCCTCGGAGACAC |
| | | | AGTCTGGGCGGTTCTCGTAAACCCGATGGTGCT |
| | | | ATTTATACTGTTGGTTCTCCTATTGATTATGGT |
| | | | GTTATTGTTGTTACTAAACCTAAGTACTCCCAG |
| | | | AACTCTGGTGATCCTCGGAGACACAGTCTGGGC |
| | | | GGTTCTCGTAAACCCGATATTATTCTTGTTAAT |
| | | | GATAATATTTCTCTTATTCTTATTCTTGTTGCT |
| | | | AAACCTTGA |
| SEQ ID NO. | Amino | Amino acid | MAAMAERPFQCRICMRNFSQSGDLTRHIRTHTG |
| 225 | acid | sequence encoded in | EKPFACDICGRKFARSDNLTTHTKIHTGSQKPF |
| | | pb76 | QCRICMRNFSRSSDLTRHIRTHTGEKPFACDIC |
| | | | GRKFARSDALTRHTKIHTGSQKPFQCRICMRNE |
| | | | SRSDALSEHIRTHTGEKPFACDICGRKFARSSN |
| | | | LTRHTKIHTGSQKPFQCRICMRNFSRSDALTTH |
| | | | IRTHTGLRGSNSGDPRRHSLGGSRKPDLIAYKN |
| | | | FDLLVIVLKPKYSQNSGDPRRHSLGGSRKPDGA |
| | | | IYTVGSPIDYGVIVVTKPKYSQNSGDPRRHSLG |
| | | | GSRKPDIILVNDNISLILILVAKP* |
| SEQ ID NO. | DNA | KRAS targeting | TTG-GAG-CTG-GTG-GCG-TAG-GCA |
| 226 | | sequence | |
| SEQ ID NO. | DNA | KRAS donor | AAAATGACTGAATATAAACTTGTGGTAGTTGGA |
| 227 | | template | GCTGGTGGCGTAGGCAAGAGTTGAGAATCCGTT |
| | | | GACGATACAGCTAATTCAGAATCATTTTGTGGA |
| | | | CGAATATGATCCAACAATAGAGGTAAATCTTGT |
| | | | TTTAA |
| SEQ ID NO. | DNA | POP133 RTPCR | GACTGAATATAAACTTGTGGTAGTTGGAGCT |
| 228 | | kras wt F | |
| SEQ ID NO. | DNA | POP134 RTPCR | TCCTCTTGACCTGCTGTGTCG |
| 229 | | kras wt R | |
| SEQ ID NO. | DNA | pb43 BCL11A DLR | ATGGCGGCGATGGCCGAGCGGCCCTTCCAGTGC |
| 230 | | zinc finger array | AGGATCTGTATGCGCAACTTTTCTCGGTCCTCC |
| | | (7 | AACCTGACCCGGCACATCAGAACCCATACAGGC |
| | | zinc-fingers) | GAAAAGCCTTTCGCCTGCGACATTTGTGGGAGA |
| | | | AAATTTGCTCGGTCCGACGCCCTGTCCGAGCAT |
| | | | ACCAAGATCCACACCGGCTCTCAGAAACCATTC |
| | | | CAGTGCCGCATTTGTATGCGGAATTTTTCCGAC |
| | | | TCCTCCGCCCTGACCACCCATATCCGCACTCAC |
| | | | ACCGGAGAGAAGCCCTTTGCTTGCGACATTTGT |
| | | | GGCAGGAAATTTGCTGACTCCTCCGACCTGTCC |
| | | | GAGCACACTAAGATCCATACTGGGTCACAGAAA |
| | | | CCTTTCCAGTGCCGGATTTGTATGAGAAACTTT |
| | | | AGCCAGTCCGGCAACCTGTCCCAGCACATCAGA |
| | | | ACCCATACAGGCGAAAAGCCTTTCGCCTGCGAC |
| | | | ATTTGTGGGAGAAAATTTGCTGACCGGTCCGAC |
| | | | CTGACCCGGCATACCAAGATCCACACCGGCTCT |
| | | | CAGAAACCATTCCAGTGCCGCATTTGTATGCGG |
| | | | AATTTTTCCCGGTCCGACAACCTGACCCGGCAC |
| | | | ATCAGAACACATACTGGGCTGAGA |
| SEQ ID NO. | DNA | pb49 Dystrophin | ATGGCGGCGATGGCCGAGCGGCCCTTCGCCTGC |
| 231 | | DLR zinc finger | GACATTTGTGGGAGAAAATTTGCTGATCAGTCC |
| | | array (10 zinc- | GGCAACCTGACCCGGCATACCAAGATCCACACC |
| | | fingers) | GGCTCTCAGAAACCATTCCAGTGCAGGATCTGT |
| | | | ATGCGCAACTTTTCTCGGTCCGACAACCTGTCC |
| | | | CAGCACATCAGAACCCATACAGGCGAAAAGCCT |
| | | | TTTGCTTGCGACATTTGTGGCAGGAAATTTGCT |
| | | | ACCTCCGGCGACCTGTCCCAGCACACTAAGATC |
| | | | CATACTGGGTCACAGAAACCTTTCCAGTGCCGC |
| | | | ATTTGTATGCGGAATTTTTCCACCTCCGGCTCC |
| | | | CTGACCCGGCATATCCGCACTCACACCGGAGAG |
| | | | AAGCCCTTTGCATGCGACATTTGTGGACGGAAA |
| | | | TTTGCTCGGTCCGACGCCCTGACCCGGCATACC |
| | | | AAGATTCACACTGGGTCTCAGAAACCTTTCCAG |
| | | | TGCAGGATTTGTATGAGAAATTTTTCCACCTCC |
| | | | GGCGACCTGTCCGAGCACATCAGAACCCATACA |
| | | | GGCGAAAAGCCTTTTGCTTGCGACATTTGTGGC |
| | | | AGGAAATTTGCTCAGTCCGGCAACCTGTCCGAG |
| | | | CACACTAAGATCCATACTGGGTCACAGAAACCT |
| | | | TTCCAGTGCCGCATTTGTATGCGGAATTTTTCC |
| | | | CAGTCCGGCGACCTGTCCCAGCACATCAGAACC |
| | | | CATACAGGCGAAAAGCCTTTTGCTTGCGACATT |
| | | | TGTGGCAGGAAATTTGCTCGGTCCTCCGCCCTG |
| | | | ACCCGGCACACTAAGATCCATACTGGGTCACAG |
| | | | AAACCTTTCCAGTGCCGCATTTGTATGCGGAAT |
| | | | TTTTCCCGGTCCGACGCCCTGTCCGAGCACATC |
| | | | AGAACACATACTGGGCTGAGA |
| SEQ ID NO. | DNA | pb53 PDCD-1 DLR | ATGGCGGCGATGGCCGAGCGGCCCTTCCAGTGC |
| 232 | | zinc finger array (7 | AGGATCTGTATGCGCAACTTTTCTCAGTCCGGC |
| | | zinc-fingers) & | GACCTGACCCGGCACATCAGAACCCATACAGGC |
| | | pb52 DLR zinc | GAAAAGCCTTTCGCCTGCGACATTTGTGGGAGA |
| | | finger array (for D | AAATTTGCTCGGTCCGACAACCTGTCCGAGCAT |
| | | unit) | ACCAAGATCCACACCGGCTCTCAGAAACCATTC |
| | | | CAGTGCCGCATTTGTATGCGGAATTTTTCCGAC |
| | | | CGGTCCGCCCTGTCCGAGCATATCCGCACTCAC |
| | | | ACCGGAGAGAAGCCCTTTGCTTGCGACATTTGT |
| | | | GGCAGGAAATTTGCTCGGTCCTCCGCCCTGTCC |
| | | | GAGCACACTAAGATCCATACTGGGTCACAGAAA |
| | | | CCTTTCCAGTGCCGGATTTGTATGAGAAACTTT |
| | | | AGCCGGTCCTCCCACCTGACCCGGCACATCAGA |
| | | | ACCCATACAGGCGAAAAGCCTTTCGCCTGCGAC |
| | | | ATTTGTGGGAGAAAATTTGCTCGGTCCGACGCC |
| | | | CTGACCCGGCATACCAAGATCCACACCGGCTCT |
| | | | CAGAAACCATTCCAGTGCCGCATTTGTATGCGG |
| | | | AATTTTTCCCGGTCCGACGCCCTGTCCGAGCAC |
| | | | ATCAGAACACATACTGGGCTGAGA |
| SEQ ID NO. | DNA | pb54 PDCD-1 DLR | ATGGCGGCGATGGCCGAGCGGCCCTTCCAGTGC |
| 233 | | zinc finger array (7 | AGGATCTGTATGCGCAACTTTTCTCAGTCCGGC |
| | | zinc-fingers) & | CACCTGACCCGGCACATCAGAACCCATACAGGC |
| | | pb52 DLR zinc | GAAAAGCCTTTCGCCTGCGACATTTGTGGGAGA |
| | | finger array (for R | AAATTTGCTCGGTCCGACGCCCTGACCCGGCAT |
| | | unit) | ACCAAGATCCACACCGGCTCTCAGAAACCATTC |
| | | | CAGTGCCGCATTTGTATGCGGAATTTTTCCACC |
| | | | TCCGGCGACCTGTCCGAGCATATCCGCACTCAC |
| | | | ACCGGAGAGAAGCCCTTTGCTTGCGACATTTGT |
| | | | GGCAGGAAATTTGCTCGGTCCTCCGACCTGACC |
| | | | CGGCACACTAAGATCCATACTGGGTCACAGAAA |
| | | | CCTTTCCAGTGCCGGATTTGTATGAGAAACTTT |
| | | | AGCCGGTCCGACCACCTGTCCCAGCACATCAGA |
| | | | ACCCATACAGGCGAAAAGCCTTTCGCCTGCGAC |
| | | | ATTTGTGGGAGAAAATTTGCTGACCGGTCCGAC |
| | | | CTGACCCGGCATACCAAGATCCACACCGGCTCT |
| | | | CAGAAACCATTCCAGTGCCGCATTTGTATGCGG |
| | | | AATTTTTCCCGGTCCGACGCCCTGTCCGAGCAC |
| | | | ATCAGAACACATACTGGGCTGAGATGA |
| SEQ ID NO. | DNA | pb64 CFTR DLR | ATGGCGGCGATGGCCGAGCGGCCCTTCGCCTGC |
| 234 | | zinc finger array (8 | GACATTTGTGGGAGAAAATTTGCTGATCAGTCC |
| | | zinc-fingers) | GGCAACCTGACCCGGCATACCAAGATCCACACC |
| | | | GGCTCTCAGAAACCATTCCAGTGCAGGATCTGT |
| | | | ATGCGCAACTTTTCTCGGTCCGACAACCTGTCC |
| | | | GAGCACATCAGAACCCATACAGGCGAAAAGCCT |
| | | | TTTGCTTGCGACATTTGTGGCAGGAAATTTGCT |
| | | | GACTCCTCCGCCCTGTCCCAGCACACTAAGATC |
| | | | CATACTGGGTCACAGAAACCTTTCCAGTGCCGC |
| | | | ATTTGTATGCGGAATTTTTCCCAGTCCGGCTCC |
| | | | CTGTCCCAGCATATCCGCACTCACACCGGAGAG |
| | | | AAGCCCTTTGCATGCGACATTTGTGGACGGAAA |
| | | | TTTGCTGACCGGTCCCACCTGACCCGGCATACC |
| | | | AAGATTCACACTGGGTCTCAGAAACCTTTCCAG |
| | | | TGCAGGATTTGTATGAGAAATTTTTCCCAGTCC |
| | | | GGCGACCTGTCCGAGCACATCAGAACCCATACA |
| | | | GGCGAAAAGCCTTTTGCTTGCGACATTTGTGGC |
| | | | AGGAAATTTGCTCGGTCCTCCGCCCTGACCCGG |
| | | | CACACTAAGATCCATACTGGGTCACAGAAACCT |
| | | | TTCCAGTGCCGCATTTGTATGCGGAATTTTTCC |
| | | | CGGTCCGACTCCCTGTCCCAGCACATCAGAACA |
| | | | CATACTGGGCTGAGA |
| SEQ ID NO. | DNA | pb74, pb75, and | ATGGCGGCGATGGCCGAGCGGCCCTTCCAGTGC |
| 235 | | pb76 KRAS DLRn | AGGATCTGTATGCGCAACTTTTCTCAGTCCGGC |
| | | D unit Zinc finger | GACCTGACCCGGCACATCAGAACCCATACAGGC |
| | | array (7 zinc- | GAAAAGCCTTTCGCCTGCGACATTTGTGGGAGA |
| | | fingers) | AAATTTGCTCGGTCCGACAACCTGACCACCCAT |
| | | | ACCAAGATCCACACCGGCTCTCAGAAACCATTC |
| | | | CAGTGCCGCATTTGTATGCGGAATTTTTCCCGG |
| | | | TCCTCCGACCTGACCCGGCATATCCGCACTCAC |
| | | | ACCGGAGAGAAGCCCTTTGCTTGCGACATTTGT |
| | | | GGCAGGAAATTTGCTCGGTCCGACGCCCTGACC |
| | | | CGGCACACTAAGATCCATACTGGGTCACAGAAA |
| | | | CCTTTCCAGTGCCGGATTTGTATGAGAAACTTT |
| | | | AGCCGGTCCGACGCCCTGTCCGAGCACATCAGA |
| | | | ACCCATACAGGCGAAAAGCCTTTCGCCTGCGAC |
| | | | ATTTGTGGGAGAAAATTTGCTCGGTCCTCCAAC |
| | | | CTGACCCGGCATACCAAGATCCACACCGGCTCT |
| | | | CAGAAACCATTCCAGTGCCGCATTTGTATGCGG |
| | | | AATTTTTCCCGGTCCGACGCCCTGACCACCCAC |
| | | | ATCAGAACACATACTGGGCTGAGA |
| SEQ ID NO. | DNA | human BCL11A | TTAAAAAATAGCTAAGAATAGTGAAAACACCCT |
| 236 | | gene Reference | TGTAATTTAGAGACTCTCAGAAAAATGACAGCA |
| | | Sequence (partial). | CCATTTAGAGCCTGGAATTACAGTTTGACTTCA |
| | | Gene ID: 53335 | CTGTGCCTTCTCTGCCCCAGGCTCCCATGGTGG |
| | | | CAAGGGTTTTTGGTTGGGGGAAGGGGTATTGAA |
| | | | TTGCCTGTCTTTGAGCAGGAAAAGAATTACAGT |
| | | | TTTCCAGGTACCTTTTGTGTGTATGTGCTGATT |
| | | | GAGGGCCCATTGAGAATATTTTGACTTTTAGGG |
| | | | AAGCTCCAAACTCTCAAACCACAGGGATCACAA |
| | | | CACATACGTGTGTCTGTTATGACGTTATATGTA |
| | | | AGCATCACAACAGGCAGAGAATGTCTGCACCCC |
| | | | ACCCTGGAAAACAGCCTGACTGTGCCCCATGGG |
| | | | CAAACCAGACTAGTTTATAGGGGGTTCTACTCT |
| | | | GAGGTACTGATGGACCTTGGGTGCTATTCCTGT |
| | | | GATAAGGAAGGCAGCTAGACAGGACTTGGGAGT |
| | | | TATCTGTAGTGAGATGGCTGAAAAGCGATACAG |
| | | | GGCTGGCTCTATGCCCCAGGTGTGCATAAGTAA |
| | | | GAGCAGATAGCTGATTCCAGTGCAAAGTCCATA |
| | | | CAGGTAATAACATAGGCCAGAAAAGAGATATGG |
| | | | CATCTACTCTTAGACATAACACACCAGGGTCAA |
| | | | TACAACTTTGAAGCTAGTCTAGTGCAAGCTAAC |
| | | | AGTTGCTTTTATCACAGGCTCCAGGAAGGGTTT |
| | | | GGCCTCTGATTAGGGTGGGGGCGTGGGTGGGGT |
| | | | AGAAGAGGACTGGCAGACCTCTCCATCGGTGGC |
| | | | CGTTTGCCCAGGGGGGCCTCTTTCGGAAGGCTC |
| | | | TCTTGGTGATGGAGAATTGGATTTTATTTCTCA |
| | | | ATGGGAATGAAATAATTTGTATGCCATGCCGTG |
| | | | TGGACTCCCAAAATTGTAAAGGAGGTGAAGCTT |
| | | | CCCCTGTCTGCACTCTCCCCTCCTCATAATTGT |
| | | | CCATTTTTCATCTGTCGGGCTGTCCACCCATCC |
| | | | ATCACATATAGGCACCTATCAGGTACCAGCTAC |
| | | | TGTGTTAGGATCTGTGTTCCCAACTGACTTGCC |
| | | | TCCCCCTGACGTCATATTCTTTTCCTTTTTCCT |
| | | | CTCCCTTTTCCCTTTTCTTCTGACCCAAACTAG |
| | | | GAATTGGGGAAAGGGCCTGATAACTTTGTTTCT |
| | | | GCTGAGGTGTAACTAATAAATACCAGGAGGCAG |
| | | | CATTTTAGTTCACAAGCTCGGAGCACTTACTCT |
| | | | GCTCTAGGAACTTTACAAATACGCACTCATTTT |
| | | | ATTTTCATACAAACCCTATGAAGCATATACTAT |
| | | | TATTATTCCTATTTTACGGATGAGTCCATATTT |
| | | | TAA |
| SEQ ID NO. | DNA | human DMD | AAGCTTGAGAGACAAGAAACATTCTTCCATTCT |
| 237 | | (Dystrophin) gene | ACTCATCTTCTTCTCTAATGAGGAGACAACCTT |
| | | Reference Sequence | AAAAGCACAGTTACATAGCCATAAAAATTAATG |
| | | (partial). Human | ATTGGCTACCTCAGAATGAAAATTCAATGTCTC |
| | | dystrophin exon 51 | ATTTTTTTTTAATATTCTTAGAATCGTTCACTG |
| | | with flanking | GTTGTCCAGTGTGAGTCTCCTGTTGAGATGTCT |
| | | sequence | TTTGCAGCTTTCCTTGAAACCTTTCATTCCAAA |
| | | | CTACATAGTCCAATAATTTTGCCACCAATCTTC |
| | | | TGGTTATATTATGCTCTTGAGTCTGTTGTCTAT |
| | | | AAACTTGATTAGGCATTCCTTCCCCTCACCACT |
| | | | CACCTCTGATAACCCAGCTGTGTGTTGGTATTT |
| | | | AGTATCAATTCACACCAGCAAGTTCAGCCCTCT |
| | | | TCAATCAATATAGGGCCACACACGGACTTTTGA |
| | | | CTGACTACTCCCCAAGTATTTCACATTTTGGGG |
| | | | CCTTATCTCCAGTTTCTCACCACAGTTGTTCAT |
| | | | CACTGTGTTTCTTACTAGCCAGGTGTTTATAAA |
| | | | AACACTAATACCTAACACTATTGATCACCTACT |
| | | | ATAGTGTCAGGCGCTGTAATAATATTATTGTGA |
| | | | TGATGATGATTATGCTGCTCTTTCTGGCATTGT |
| | | | CATACGTGTATTGCTTGTACTACTCACTGAATC |
| | | | TACACAACTGCCCTTATGACATTTACCCTGTTA |
| | | | TTATTCCTCTTTTAAGGTAAATACATGAAAAAT |
| | | | GCTTCCCACTTTGCCTTGCTTACTGCTTATTGC |
| | | | TAGTACTGAACAAATGTTAGAACTGAAACTTAG |
| | | | AGAGGTTATGTGGCTTTACCAAGGTCCCAGAGT |
| | | | TCCTAGGGTAGAGAACAGGATTGTCTACCAGAC |
| | | | ATTTTAATTCTAGTACTATGCATCTTAACCATT |
| | | | ACCATAGGCTGACTTACTCTACAGTGTCCAACA |
| | | | TATTCACTATTAAGATTTATTTAATGACTTTGA |
| | | | AACAGTATTTCATGTCTAAATAGAAAAACTACT |
| | | | AACTCGCATTTTTAAGAAAATATTGTATCTTGG |
| | | | TTTTTCTTCACTGCTGGCCAGTTTACTAACAAT |
| | | | CTGAAATAAAAAGAAAAAAATATGATAAACTGC |
| | | | TCCCAGTATAAAATACAGAGCTAAGACAAGAAC |
| | | | GTTTCATTGGCTTTGATTTCCCTAGGGTCCAGC |
| | | | TTCAAATTAATTTACTTCCTATTCAAGGGAATT |
| | | | CTTAAATCAGAAAGAAGATCTTATCCCATCTTG |
| | | | TTTTGCCTTTGTTTTTTCTTGAATAAAAAAAAA |
| | | | ATAAGTAAAATTTATTTCCCTGGCAAGGTCTGA |
| | | | AAACTTTTGTTTTCTTTACCACTTCCACAATGT |
| | | | ATATGATTGTTACTGAGAAGGCTTATTTAACTT |
| | | | AAGTTACTTGTCCAGGCATGAGAATGAGCAAAA |
| | | | TCGTTTTTTAAAAAATTGTTAAATGTATATTAA |
| | | | TGAAAAGGTTGAATCTTTTCATTTTCTACCATG |
| | | | TATTGCTAAACAAAGTATCCACATTGTTAGAAA |
| | | | AAGATATATAATGTCATGAATAAGAGTTTGGCT |
| | | | CAAATTGTTACTCTTCAATTAAATTTGACTTAT |
| | | | TGTTATTGAAATTGGCTCTTTAGCTTGTGTTTC |
| | | | TAATTTTTCTTTTTCTTCTTTTTTCCTTTTTGC |
| | | | AAAAACCCAAAATATTTTAGCTCCTACTCAGAC |
| | | | TGTTACTCTGGTGACACAACCTGTGGTTACTAA |
| | | | GGAAACTGCCATCTCCAAACTAGAAATGCCATC |
| | | | TTCCTTGATGTTGGAGGTACCTGCTCTGGCAGA |
| | | | TTTCAACCGGGCTTGGACAGAACTTACCGACTG |
| | | | GCTTTCTCTGCTTGATCAAGTTATAAAATCACA |
| | | | GAGGGTGATGGTGGGTGACCTTGAGGATATCAA |
| | | | CGAGATGATCATCAAGCAGAAGGTATGAGAAAA |
| | | | AATGATAAAAGTTGGCAGAAGTTTTTCTTTAAA |
| | | | ATGAAGATTTTCCACCAATCACTTTACTCTCCT |
| | | | AGACCATTTCCCACCAGTTCTTAGGCAACTGTT |
| | | | TCTCTCTCAGCAAACACATTACTCTCACTATTC |
| | | | AGCCTAAGTATAATCAAGGATATAAATTAATGC |
| | | | AAATAACAAAAGTAGCCATACATTAAAAAGGAA |
| | | | ATATACAAAAAAAAAAAAAAAAAAAAGCAGAAA |
| | | | CCTTACAAGAATAGTIGTCTCAGTTAAATTTAC |
| | | | TAAACAACCTGGTATTTTAAAAATCTATTTTAT |
| | | | ACCAAATAAGTCACTCAACTGAGCTATTTACAT |
| | | | TTAAACTGTTTGTTTTGGACTACGCAGCCCAAC |
| | | | ATATTGCAGAATCAAATATAATAGTCTGGGAAT |
| | | | TGATTATTATCCACTCTTCTAAGTIGTCTGTGC |
| | | | CAATTTGCCTTCTCCAATGATAAGGATAATTGA |
| | | | AAGAGAGCTATAACTTAAAAAGAGAAAAGTAAC |
| | | | AAAACATAAGATATTTAAAATTACCCTAGATCT |
| | | | TAAAGTTGGCATTTATGCAATGCCATGTTCAAA |
| | | | TGAACATGTTTTTAATACAAATAGTGCATTTTT |
| | | | CAGCCTCAGTGTAATCCATTTGGTAAAATTATG |
| | | | ACATCAACTAGAAACATTAGAATACATTGATGT |
| | | | AAATATGGTTTACCTAGCTAGATCAAATATACT |
| | | | ATATATCTTTTATATTTGTGAATGGTTAAGAAA |
| | | | AATAATGTTGGAATTGTTATACATTAAAGTTTT |
| | | | TTCACTTGTAACAGCTTTCAAGCCTTTCTAAAG |
| | | | AAATACAAAGTTGTGCTGAAGGTATTTAGGTAT |
| | | | TAAAGTACTACCTTTTGAAAAAACAAGAAGTGA |
| | | | GGCAGACAGAGTAAGGGGAATTTCTTTGTAAAA |
| | | | TAAACTTCACCAATTCCATAGGAATAAAAGTAA |
| | | | TTTGATAGTAAACAACCTGCATTTAAAGGCCTT |
| | | | GAGCTTGAATACAGAAGACCTGAATTCAGTGCC |
| | | | ATTTGCAAATGATGATTGTGGTCAAGCCATCTC |
| | | | TGGATCTTCGTTTCCTATTCTGAGTACAGAGCA |
| | | | TACAGAGTACACATTCACATTCACAATATAGIT |
| | | | ATGGATATGGATGTATATAAATATATGTAAATA |
| | | | CTACATATATGTACCTAAAATTTGTTTTACTTC |
| | | | TGCTTTAAAAAAAGTAATTATAGCCACATTTTT |
| | | | CAGAAAAAGTAACTGAGGCTCATAGATGTCAAA |
| | | | TTCCCAGTAAGTAGCAGAACAAGGATTCAAATC |
| | | | CAAGTCCATTTGATTCCTAAGCTT |
| SEQ ID NO. | DNA | Human PDCD-1 | GATCTGGAACTGTGGCCATGGTGTGAAGGCCAT |
| 238 | | gene Reference | CCACAAGGTGGAAGCTTTGAGGGGGAGCCGATT |
| | | Sequence (partial). | AGCCATGGACAGTTGTCATTCAGTAGGGTCACC |
| | | Gene ID: 5133 | TGTGCCCCAGCGAAGGGGGATGGGCCGGGAAGG |
| | | | CAGAGGCCAGGCACCTGCCCCCAGCAGGGGCAG |
| | | | AGGCTGTGGGCAGCCGGGAGGCTCCCAGAGGCT |
| | | | CCGACAGAATGGGAGTGGGGTTGAGCCCACCCC |
| | | | TCACTGCAGCCCAGGAACCTGAGCCCAGAGGGG |
| | | | GCCACCCACCTTCCCCAGGCAGGGAGGCCCGGC |
| | | | CCCCAGGGAGATGGGGGGGATGGGGGAGGAGAA |
| | | | GGGCCTGCCCCCACCCGGCAGCCTCAGGAGGGG |
| | | | CAGCTCGGGCGGGATATGGAAAGAGGCCACAGC |
| | | | AGTGAGCAGAGACACAGAGGAGGAAGGGGCCCT |
| | | | GAGCTGGGGAGACCCCCACGGGGTAGGGCGTGG |
| | | | GGGCCACGGGCCCACCTCCTCCCCATCTCCTCT |
| | | | GTCTCCCTGTCTCTGTCTCTCTCTCCCTCCCCC |
| | | | ACCCTCTCCCCAGTCCTACCCCCTCCTCACCCC |
| | | | TCCTCCCCCAGCACTGCCTCTGTCACTCTCGCC |
| | | | CACGTGGATGTGGAGGAAGAGGGGGCGGGAGCA |
| | | | AGGGGCGGGCACCCTCCCTTCAACCTGACCTGG |
| | | | GACAGTTTCCCTTCCGCTCACCTCCGCCTGAGC |
| | | | AGTGGAGAAGGCGGCACTCTGGTGGGGCTGCTC |
| | | | CAGGCATGCAGATCCCACAGGCGCCCTGGCCAG |
| | | | TCGTCTGGGCGGTGCTACAACTGGGCTGGCGGC |
| | | | CAGGATGGTTCTTAGGTAGGTGGGGTCGGCGGT |
| | | | CAGGTGTCCCAGAGCCAGGGGTCTGGAGGGACC |
| | | | TTCCACCCTCAGTCCCTGGCAGGTCGGGGGGTG |
| | | | CTGAGGCGGGCCTGGCCCTGGCAGCCCAGGGGT |
| | | | CCCGGAGCGAGGGGTCTGGAGGGACCTTTCACT |
| | | | CTCAGTCCCTGGCAGGTCGGGGGGTGCTGTGGC |
| | | | AGGCCCAGCCTTGGCCCCCAGCTCTGCCCCTTA |
| | | | CCCTGAGCTGTGTGGCTTTGGGCAGCTCGAACT |
| | | | CCTGGGTTCCTCTCTGGGCCCCAACTCCTCCCC |
| | | | TGGCCCAAGTCCCCTCTTTGCTCCTGGGCAGGC |
| | | | AGGACCTCTGTCCCCTCTCAGCCGGTCCTTGGG |
| | | | GCTGCGTGTTTCTGTAGAATGACGGGTCAGGCT |
| | | | GGCCAGAACCCCAAACCTTGGCCGTGGGGAGTC |
| | | | TGCGTGGCGGCTCTGCCTTGCCCAGGCATCCTT |
| | | | GGTCCTCACTCGAGTTTTCCTAAGGATGGGATG |
| | | | AGCCCCATGTGGGACTAACCTTGGCTTTACGAC |
| | | | GTCAAAGTTTAGATGAGCTGGTGATATTTTTCT |
| | | | CATTATATCCAAAGTGTACCTGTTCGAGTGAGG |
| | | | ACAGTTCTTCTGTCTCCAGGATCCCTCCTGGGT |
| | | | GGGGATTGTGCCCGCCTGGGTCTCTGCCCAGAT |
| | | | TCCAGGGCTCTCCCCGAGCCCTGTTCAGACCAT |
| | | | CCGTGGGGGAGGCCTTGGCCTTACTCTCCCGGA |
| | | | TCGAGGAGAGAGGGAGCCTCTTCCTGGGCTGCC |
| | | | CGTGACCCTGGGCCCTCTGTGTACACTGTGACC |
| | | | ACAGCCCGCTCCTGGACCCTCTGTGCCCGGCTG |
| | | | GCCCTCTGTGCCCAGCCAGCCTGCACCTGGGGA |
| | | | TGCCAAGGCCTGGGGAGGGTGGTTTCACCCAGG |
| | | | CCAAGCCTAAGACAGTCCCTCTGGGCCCTGCTG |
| | | | GGTACCGGGGTGTGACACCACTGGGAGGACAAG |
| | | | ATGAGGGGCACCCCTGGGGCCGCCCTGACACCC |
| | | | CCTTGAGGCTCCTGCCCCGGGGGTCCTGGTGCC |
| | | | CCTTCACTGTGGCAGGCGACTGGGGGTTCCCCA |
| | | | CCTCGGCCCCTCTCCCGGGGCCTGCTCCCCGGC |
| | | | ACCTGAGGCAGCATCCTTGTCAGGGCCGTGCCT |
| | | | TCCTGCCTCAGCGCCACCTCTTAAGGTTGGCCC |
| | | | GTGGGTCACTCAGGACTCACAACTGGAGATTCT |
| | | | GGGCAAAAGGCAAAGAGCAA |
| SEQ ID NO. | DNA | Human CFTR gene | CACTGTAGCTGTACTACCTTCCATCTCCTCAAC |
| 239 | | Reference Sequence | CTATTCCAACTATCTGAATCATGTGCCCTTCTC |
| | | (partial). Gene | TGTGAACCTCTATCATAATACTTGTCACACTGT |
| | | ID:1080 | ATTGTAATTGTCTCTTTTACTTTCCCTTGTATC |
| | | | TTTTGTGCATAGCAGAGTACCTGAAACAGGAAG |
| | | | TATTTTAAATATTTTGAATCAAATGAGTTAATA |
| | | | GAATCTTTACAAATAAGAATATACACTTCTGCT |
| | | | TAGGATGATAATTGGAGGCAAGTGAATCCTGAG |
| | | | CGTGATTTGATAATGACCTAATAATGATGGGTT |
| | | | TTATTTCCAGACTTCACTTCTAATGATGATTAT |
| | | | GGGAGAACTGGAGCCTTCAGAGGGTAAAATTAA |
| | | | GCACAGTGGAAGAATTTCATTCTGTTCTCAGTT |
| | | | TTCCTGGATTATGCCTGGCACCATTAAAGAAAA |
| | | | TATCATCTTTGGTGTTTCCTATGATGAATATAG |
| | | | ATACAGAAGCGTCATCAAAGCATGCCAACTAGA |
| | | | AGAGGTAAGAAACTATGTGAAAACTTTTTGATT |
| | | | ATGCATATGAACCCTTCACACTACCCAAATTAT |
| | | | ATATTTGGCTCCATATTCAATCGGTTAGTCTAC |
| | | | ATATATTTATGTTTCCTCTATGGGTAAGCTACT |
| | | | GTGAATGGATCAATTAATAAAACACATGACCTA |
| | | | TGCTTTAAGAAGCTTGCAAACACATGAAATAAA |
| | | | TGCAATTTATTTTTTAAATAATGGGTTCATTTG |
| | | | ATCACAATAAATGCATTTTATGAAATGGTGAGA |
| | | | ATTTTGTTCACTCATTAGTGAGACAAACGTCTC |
| | | | AATGGTTATTTATATGGCATGCATATAGTGATA |
| | | | TGTGGT |
| SEQ ID NO. | DNA | Human KRAS gene | TATGATCCTTTGAGAGCCTTTAGCCGCCGCAGA |
| 240 | | Reference Sequence | ACAGCAGTCTGGCTATTTAGATAGAACAACTTG |
| | | (partial). Gene | ATTTTAAGATAAAAGAACTGTCTATGTAGCATT |
| | | ID:3845 | TATGCATTTTTCTTAAGCGTCGATGGAGGAGTT |
| | | | TGTAAATGAAGTACAGTTCATTACGATACACGT |
| | | | CTGCAGTCAACTGGAATTTTCATGATTGAATTT |
| | | | TGTAAGGTATTTTGAAATAATTTTTCATATAAA |
| | | | GGTGAGTTTGTATTAAAAGGTACTGGTGGAGTA |
| | | | TTTGATAGTGTATTAACCTTATGTGTGACATGT |
| | | | TCTAATATAGTCACATTTTCATTATTTTTATTA |
| | | | TAAGGCCTGCTGAAAATGACTGAATATAAACTT |
| | | | GTGGTAGTTGGAGCTGGTGGCGTAGGCAAGAGT |
| | | | GCCTTGACGATACAGCTAATTCAGAATCATTTT |
| | | | GTGGACGAATATGATCCAACAATAGAGGTAAAT |
| | | | CTTGTTTTAATATGCATATTACTGGTGCAGGAC |
| | | | CATTCTTTGATACAGATAAAGGTTTCTCTGACC |
| | | | ATTTTCATGAGTACTTATTACAAGATAATTATG |
| | | | CTGAAAGTTAAGTTATCTGAAATGTACCTTGGG |
| | | | TTTCAAGTTATATGTAACCATTAATATGGGAAC |
| | | | TTTACTTTCCTTGGGAGTATGTCAGGGTCCATG |
| | | | ATGTTCACTCTCTGTGCATTTTGATTGGAAGTG |
| | | | TATTTCAGAGTTTCGTGAGAGGGTAGAAATTTG |
| | | | TATCCTATCTGGACCTAAAAGACAATCTTTTTA |
| | | | TTGTAACTTTTATTTTTATGGGTTTCTTGGTAT |
| | | | TGTGACATCATATGTAAAGGTTAGATTTAATTG |
| | | | TACTAGTGAAATATAATTGTTTGATGGTTGATT |
| | | | TTTTTAAACTTCATCAGCAGTATTTTCCTATCT |
| | | | TCTTCTCAACATTAGAGAACCTACAACTACCGG |
| | | | ATAAATTTTACAAAATGAATTATTTGCCTAAGG |
| | | | TGTGGTTTATATAAAGGTACTATTACCAACTTT |
| | | | ACCTTTGCTTTGTTGTCATTTTTAAATTTACTC |
| | | | AAGGAAATACTAGGATTTAAAAAAAAATTCCTT |
| | | | GAGTAAATTTAAATTGTTATCATGTTTTTGAGG |
| | | | ATTATTTTCAG |
| SEQ ID NO. | DNA | Sequence | TGAGAATCCG |
| 241 | | modification | |
| | | polynucleotide | |
| SEQ ID NO. | Amino | Linker | GGGSn, where n is 1 or more |
| 242 | Acid | | (e.g., n is 1, 2, 3, 4, 5 or |
| | | | more) |
| SEQ ID NO. | Amino | Amino Acid | NSGDP |
| 243 | Acid | sequence preceding | |
| | | beta sheet 1 | |
| SEQ ID NO. | Amino | Linker | GGGGGSn, where n is 1, 2, 3, 4, |
| 244 | Acid | | 5, 6, 7, 8 or more |
| SEQ ID NO. | DNA | Sequence | TTAGACTCT |
| 245 | | modification | |
| | | polynucleotide | |
| SEQ ID NO. | Amino | Wild type CFTR | NIIFGV |
| 246 | Acid | amino acids codons | |
| | | 505-510 | |

1.-111. (canceled)

112. A polymeric modification agent comprising a structure represented by:
D-L-Rn,
wherein the D element is or comprises a sequence-specific binding element;
the L element is optional and is or comprises a linker element; the R element is or comprises a binding element that is optionally sequence-specific, and n equals 1, 2, or 3, and
wherein the R element has a binding affinity with a dissociation constant of 10E-3 or lower for at least one target site.

113. The polymeric modification agent of claim 112, wherein the D element binds to a landing site adjacent to or overlapping with the at least one target site on a single strand on a first polynucleotide, and the R element binds to the at least one target site on a single strand on a second polynucleotide, wherein each of the first and second polynucleotides may be part of the same or different molecules.

114. The polymeric modification agent of claim 112, wherein neither the D element, L element, or R element results in a reduction in speed of DNA replication within the vicinity of the target site.

115. The polymeric modification agent of claim 112, wherein the D element is or comprises a polypeptide, wherein the D element is or comprises a polypeptide between 80 and 10,000 amino acids in length or 8 kD and 1,000 kD in size, and/or wherein the sequence of the D element is at least 50% identical to any one of SEQ ID NOs: 2, 3, 5, 7, 9, 11, 12, 161, 162, 174, 175, 181, 184, 187, 188, 189, 196, 197, 219, 222, 225, or 226.

116.-117. (canceled)

118. The polymeric modification agent of claim 112, wherein the D element is or comprises a polynucleotide, wherein the polynucleotide is between 20 and 50,000 nucleotides in length, wherein the sequence of the D element comprises a nucleotide sequence at least 50% identical to any one of SEQ ID NOs: 91, 92, 93, 94, 95, 96, 97, 230, 231, 232, 233, 234, or 235, and/or wherein the polynucleotide comprises one or more chain(s) of polynucleotide(s).

119. (canceled)

120. A composition comprising the polymeric modification agent of claim 112 and a sequence modification polynucleotide.

121.-122. (canceled)

123. The polymeric modification agent of claim 112, wherein the L element is or comprises a polypeptide, wherein the L element is or comprises a polypeptide between 2 and 100 amino acids in length or 0.2 kD and 10 kD in size, and/or wherein the sequence of the L element comprises a sequence at least 50% identical to any one of SEQ ID NOs: 1, 13, or 14.

124.-125. (canceled)

126. The polymeric modification agent of claim 112, wherein the L element is or comprises a polynucleotide, wherein the L element is or comprises a polynucleotide between 2 and 500 nucleic acids in length, wherein the sequence of the L element comprises a sequence at least 50% identical to any one of SEQ ID NOs: 98, 99, or 100, and/or wherein the polynucleotide comprises one or more chain(s) of polynucleotide(s).

127.-129. (canceled)

130. The polymeric modification agent of claim 112, wherein the R element is or comprises a polypeptide, wherein the R element is or comprises a polypeptide between 10 and 50,000 amino acids in length or 1 kD and 5,000 kD in size and/or wherein the sequence of the R element comprises a sequence at least 50% identical to a sequence selected from any one of SEQ ID NOs 19, 81, 84, 101-128, 208, 210, 212, 214, or 216.

131-132. (canceled)

133. The polymeric modification agent of claim 112, wherein the R element is or comprises a polynucleotide comprising one or more polynucleotide chain(s), wherein the R element is or comprises a polynucleotide between 2 and 50,000 nucleic acids in length, and/or wherein the sequence of the R element comprises a sequence at least 50% identical to any one of SEQ ID NOs: 20, 85, 129-156, 207, 209, 211, 213 or 215.

134.-141. (canceled)

142. The polymeric modification agent of claim 112, wherein the agent does not itself modify a target site or target sequence and/or does not cause modification of a non-target site.

143. The polymeric modification agent of claim 112, wherein the D element has a binding affinity with a dissociation constant of 10E-6 or lower for at least one target site.

144. (canceled)

145. A method comprising a step of:
contacting DNA or a cell comprising DNA with a polymeric modification agent of claim 112, wherein:
(a) the DNA includes at least one target site;
(b) the D element of the polymeric modification agent associates with a landing site adjacent to or overlapping with the at least one target site that includes at least one target sequence; and
(c) the one, two, or three R-elements bind to one strand of the DNA at the at least one target site,
wherein there is a reduced mRNA level of a target after the contacting relative to a cell that is not contacted with the polymeric modification agent of claim 112,
wherein the agent does not directly catalyze single and/or double-stranded DNA breaks,
wherein after the contacting, there is a reduction in transcription activity of the target, and/or
wherein the at least one target site is an error site.

146.-147. (canceled)

148. The method of claim 145, further comprising use of an enhancing agent and/or an inhibiting agent, wherein use of the enhancing and/or inhibiting agent enhances recombination events in DNA contacted with the polymeric modification agent, but the enhancing agent and/or inhibiting agent itself does not contact the DNA, and/or wherein the enhancing agent and/or inhibiting agent comprises RNAi activity.

149.-154. (canceled)

155. The method of claim 145, wherein
(i) the DNA is being actively transcribed;
(ii) the step of contacting occurs within the context of an RNA polymerase;
(iii) the DNA is actively replicating;
(iv) the step of contracting occurs within the context of a DNA replication fork;
(v) the step of contacting results in reduced speed of DNA replication; and/or
(vi) the step of contacting results in reduced speed of DNA replication within the vicinity of the at least one target site.

156.-170. (canceled)

171. The method of claim 145, wherein the at least one target site is modified, and/or wherein the modification is confirmable by polymerase chain reaction analysis, Sanger sequencing, or next generation sequencing.

172.-185. (canceled)

186. The polymeric modification agent of claim 112, wherein the D element is or comprises a dCas9.

187. The polymeric modification agent of claim 112, wherein neither the D element, L element, or R element acts primarily as a nuclease.

188. The method of claim 145, further comprising a step of:
contacting the DNA or the cell comprising DNA with a sequence modification polynucleotide that
(i) binds specifically to one strand of the DNA at the at least one target site, and
(ii) has a mismatch or other DNA sequence difference relative to the at least one target site,
wherein the sequence modification polynucleotide incorporates a sequence modification into a complement of the one strand of the DNA, and/or
wherein incorporation of the sequence modification into the complement of the one strand of the DNA occurs at a frequency of two to ten times greater than a frequency of incorporation of the sequence modification into the complement of the one strand of the DNA that occurs in the absence of the enhancing agent and/or inhibiting agent.

189. The method of claim 188, further comprising contacting the DNA or the cell comprising DNA with a DNA polymerase, helicase, ligase, recombinase, repair scaffold protein, single strand DNA binding protein, or mismatch repair protein.

190. The method of claim 188, wherein the sequence modification polynucleotide comprises one or more deletion(s), substitution(s), or insertion(s), relative to the target sequence, wherein the sequence modification polynucleotide is between 10 and 20,000 nucleotides in length.

191. The method of claim 188, wherein the sequence modification polynucleotide is or comprises a sequence at least 70% identical to any one of SEQ ID NOs: 22, 23, 29-33, 157, 163, 176, 190, 198, or 226.

192. The method of claim 188, further comprising a step of administrating at least one additional agent that induces DNA replication or induces DNA breakage.
